(19) United States
(12) Patent Application Publication
(10) Pub. No.: US 2024/0279207 A1
(43) Pub. Date: Aug. 22, 2024

(54) SUBSTITUTED ACYL SULFONAMIDES FOR TREATING CANCER

(71) Applicants: The Broad Institute, Inc., Cambridge, MA (US); Bayer Aktiengesellschaft, Leverkusen (DE)

(72) Inventors: Léa Bouché, Berlin (DE); Daniel Korr, Berlin (DE); Antonius ter Laak, Berlin (DE); Michael Kröber, Berlin (DE); Naomi Barak, Berlin (AD); Roman Hillig, Berlin 13353 (DE); Roland Neuhaus, Berlin (DE); Stefan Gradl, Berlin (DE); Jörg Wichard, Berlin (DE); Ernesto Amaury Fernamdez-Montalvan, Leverkusen (DE); Matyas Gorjanacz, Berlin (DE); Vera Pütter, Berlin (DE); Andreas Stutter, Berlin (DE); Steven James Ferrara, Cambridge, MA (US)

(21) Appl. No.: 18/032,067

(22) PCT Filed: Oct. 14, 2021
(86) PCT No.: PCT/US21/54921

§ 371 Date: Apr. 14, 2023

Related U.S. Application Data
(60) Provisional application No. 63/092,935, filed on Oct. 16, 2020.

(51) Int. Cl. C07D 405/04; A61K 31/397; A61K 31/4025; A61K 31/404; A61K 31/4155; A61K 31/4192; A61K 31/422; A61K 31/4245; A61K 31/427; A61K 31/433; A61K 31/437; A61K 31/443; A61K 31/4525; A61K 31/4709; A61K 31/4725; A61K 31/496; A61K 31/498; A61K 31/5025; A61K 31/5365; A61K 31/5377; A61K 31/539; A61P 35/00; C07D 405/14; C07D 409/14; C07D 413/14; C07D 417/14; C07D 471/04; C07D 487/04; C07D 491/107; C07D 498/04

(52) U.S. Cl. CPC C07D 405/04; A61K 31/397; A61K 31/4025; A61K 31/404; A61K 31/4155; A61K 31/4192; A61K 31/422; A61K 31/4245; A61K 31/427; A61K 31/433; A61K 31/437; A61K 31/443; A61K 31/4525; A61K 31/4709; A61K 31/4725; A61K 31/496; A61K 31/498; A61K 31/5025; A61K 31/5365; A61K 31/5377; A61K 31/539; A61P 35/00; C07D 405/14; C07D 409/14; C07D 413/14; C07D 417/14; C07D 471/04; C07D 487/04; C07D 491/107; C07D 498/04

(57) ABSTRACT

The present invention provides acyl sulfonamide compounds of general formula (I):

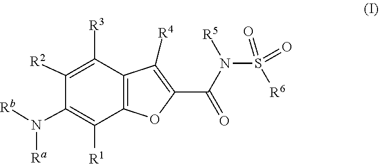

in which R¹, R², R³, R⁴, R⁵, R⁶, R^{a} and R^{b} are as described and defined herein, methods of preparing said compounds, intermediate compounds useful for preparing said compounds, pharmaceutical compositions and combinations comprising said compounds, and the use of said compounds for manufacturing pharmaceutical compositions for the treatment or prophylaxis of diseases, in particular of hyperproliferative disorders, as a sole agent or in combination with other active ingredients as well as methods of treatment and/or prophylaxis of diseases, particularly cancer, more particularly cancer in which KAT6A and/or KAT6B is focally amplified, said method comprising administering an effective amount of at least one compound of formula (I) to a subject in need thereof.

### RELATED APPLICATION

This application claims the benefit of U.S. Provisional Application No. 63/092,935, filed Oct. 16, 2020, the contents of which is fully incorporated by reference herein.

### BACKGROUND

The present invention provides acyl sulfonamide compounds of general formula (I) which inhibit the activity of lysine acetyl transferase 6A (KAT6A) and lysine acetyl transferase 6B (KAT6B). In particular, the present invention provides compositions and methods for the treatment of lysine acetyl transferase 6A (KAT6A) activated cancers and lysine acetyl transferase 6B (KAT6B) activated cancers. More particularly, the present disclosure provides inhibitors of KAT6A and KAT6B for the treatment of breast cancer, lung cancer, ovarian cancer, endometrial cancer, esophageal cancer, bladder cancer, and acute myeloid leukemia. Even more particularly, the present disclosure provides inhibitors of KAT6A and KAT6B for the treatment of lung cancer, breast cancer, bladder cancer, uterine cancer, endometrial cancer, prostate cancer and leukemia.

Epigenetic regulation of gene expression is a complex process which confers stable, long term and, in some cases, heritable alterations in cellular gene expression. Rather than involving changes to the actual DNA sequence of the gene, epigenetic regulation is mediated through modifications to either the DNA backbone or to its associated chromatin proteins. These modifications facilitate interaction with the epigenetic gene regulation machinery, with the end result being stable gene activation/expression or stable gene silencing/repression. Alterations to the epigenetic gene regulation machinery underlie many different and diverse disease pathologies, and this is now recognized as a major driver in the oncogenic process for most cancers.

Histone proteins are the key chromatin proteins that facilitate the effector functions of the epigenetic gene regulation machinery. These are a family of five closely related proteins, denoted as histones H1, H2A, H2B, H3, and H4, that package and order the DNA into structural units called nucleosomes. Histones H3 and H4 both feature long amino-terminal polypeptide tails that protrude from the nucleosome, and that are rich in lysine and arginine amino acid residues which confer a net positive charge that enables the histone tails to interact with and bind to the negatively charged phosphate groups of the DNA backbone. Post-translational modification (PTM) of the lysine and arginine residues alters the nucleosome structure by altering the ability of the histones to bind DNA, and also by providing specific binding sites for the epigenetic gene regulation machinery. Such PTMs include methylation, acetylation, phosphorylation, ubiquitylation, and sumoylation.

Histone acetyltransferases (HATs) comprise a discrete family of enzymes which catalyze the transfer of an acetyl group from acetyl coenzyme A to a lysine residue in histones as well as other protein substrates. Lysine acetyl transferase 6A (KAT6A) and lysine acetyl transferase 6B (KAT6B) are the two HATs which are the focus of this disclosure. Both proteins have been implicated in cancer. KAT6A is the target of recurrent chromosomal translocations in acute myeloid leukemia, and it is focally amplified in lung, breast, ovarian, endometrial, bladder, and esophageal cancers. Similarly, KAT6B chromosomal translocations have been identified in a diverse range of cancers, and it is focally amplified in breast, ovarian, uterine, stomach, bladder, and lung cancer. Expression of KAT6A and KAT6B correlates well with gene copy number in tumors that have these focal amplifications, suggesting that there has been selective pressure to maintain their activity during the oncogenic process. Moreover, cancer cell lines derived from tumors which bear these focal amplifications have high levels of KAT6A or KAT6B expression and are genetically dependent on this activity in long term proliferation experiments.

The biological pathways impacted by KAT6A and KAT6B activity are poorly defined, although there is some data which supports a gene activation function, notably the control of ESR1 expression in breast cancer cell lines. Similarly, the histone substrates of KAT6A and KAT6B are also poorly defined, and are thought to include lysine 9 of histone H3 (H3K9), lysine 14 of histone 3 (H3K14) and lysine 23 of histone H3 (H3K23). Acetylation at these positions is associated with such diverse functions as gene activation and DNA damage recognition, and it remains unclear which of these PTMs are the critical effectors in tumor cells that are dependent on KAT6A or KAT6B.

WO2016198507 discloses aryl sulfonhydrazide derivatives known as MOZ (KAT6A) inhibitors. Further, WO2019108824 relates to sulfonohydrazido compounds as histone acetyltransferase inhibitors. However, there are currently no FDA-approved targeted therapeutics for KAT6A or KAT6B. Accordingly, there is an urgent need for compounds, compositions and methods for treating KAT6A- or KAT6B-activated cancers.

### SUMMARY

The present invention provides acyl sulfonamide compounds of general formula (I):

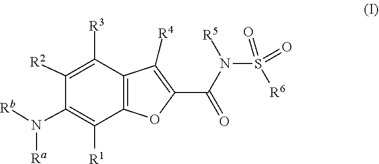

in which R¹, R², R³, R⁴, R⁵, R⁶, R^{a} and R^{b} are as described and defined herein, methods of preparing said compounds, intermediate compounds useful for preparing said compounds, pharmaceutical compositions and combinations comprising said compounds, and the use of said compounds for manufacturing pharmaceutical compositions for the treatment or prophylaxis of diseases, in particular of hyperproliferative disorders, as a sole agent or in combination with other active ingredients.

### DESCRIPTION OF THE INVENTION

It has now been found that the compounds of the present invention effectively inhibit the activity of lysine acetyl transferase 6A (KAT6A) and/or lysine acetyl transferase 6B (KAT6B) for which data are given in the biological experimental section, and may therefore be used for the treatment or prophylaxis of hyperproliferative disorders, such as cancer disorders.

In accordance with a first aspect, the present invention provides compounds of general formula (I):

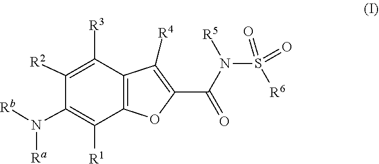

wherein
R¹ is a hydrogen atom;
R² is a hydrogen atom;
R³ is selected from a hydrogen atom and a fluorine atom;
R⁴ is a hydrogen atom;
R⁵ is a hydrogen atom;
R⁶ is selected from a C₁-C₆-alkyl group, a phenyl group, a naphthyl group, a heteroaryl group, a (phenyl)-(C₁-C₆-alkyl)- group, a (naphthyl)-(C₁-C₆-alkyl)- group, a (heteroaryl)-(C₁-C₆-alkyl)- group, a (heterocycloalkyl)-(C₁-C₆-alkyl)- group and a heterocycloalkyl group,
   wherein said heterocycloalkyl group is connected to the rest of the molecule via a carbon atom of said heterocycloalkyl group,
   wherein said phenyl, naphthyl, heteroaryl or heterocycloalkyl group is each optionally substituted with one or more substituents independently selected from a halogen atom, a cyano group, a nitro group, a hydroxy group, a C₁-C₆-alkyl group, a C₁-C₆-hydroxyalkyl group, a C₁-C₆-haloalkyl group, a C₁-C₆-alkoxy group, a (C₁-C₃-alkyl)-O—(C₁-C₂-alkyl)- group, a (C₁-C₃-alkyl)-O—(C₁-C₂-alkyl)-O— group, a (C₃-C₃-cycloalkyl)-(C₁-C₂-alkyl)-O— group, a (C₃-C₃-halocycloalkyl)-(C₁-C₂-alkyl)-O— group, a (phenyl)-(C₁-C₂-alkyl)-O— group, a C₁-C₆-haloalkoxy group, a C₂-C₆-alkenyl group, a C₂-C₆-alkynyl group, a C₃-C₃-cycloalkyl group, a C₄-C₃-cycloalkenyl group, a C₃-C₃-cycloalkoxy group, a C₁-C₆-thioalkyl group, a heterocycloalkyl group, a R⁷R⁸N— group, a (C₁-C₂-alkyl)-S(═O)₂— group, a (R⁷R⁸N)—S(═O)₂— group, a (R⁷R⁸N)—(C₁-C₆-alkyl)-(C═O)—NH— group, a (H₃C)—(C═O)— group, a (R⁷R⁸N)—(C═O)— group, a (R⁷R⁸N)—(C═O)—O— group, a (R⁷R⁸N)—(C₁-C₆-alkyl)- group, a R⁹OOC—(C₁-C₃-alkyl)- group, a R⁹OOC— group, a phenyl group, a naphthyl group, a (phenyl)-O— group and a heteroaryl group,
      wherein said heterocycloalkyl group is connected to the rest of the molecule via a carbon atom of said heterocycloalkyl group,
      wherein said phenyl, naphthyl, heteroaryl or heterocycloalkyl group is each optionally substituted with one or more substituents independently selected from a halogen atom, a cyano group, a nitro group, a C₁-C₆-alkyl group, a C₁-C₆-hydroxyalkyl group, a C₁-C₆-alkylsulfanyl group, a C₁-C₆-haloalkyl group, a C₁-C₆-alkoxy group, a C₁-C₆-haloalkoxy group, a C₂-C₆-alkenyl group, a C₂-C₆-alkynyl group, a C₃-C₃-cycloalkyl group, a C₄-C₃-cycloalkenyl group, a C₃-C₃-cycloalkoxy group, a heterocycloalkyl group, a phenyl group, a R⁷R⁸N— group, a (R⁷R⁸N)—S(═O)₂— group, a (R⁷R⁸N)—(C₁-C₆-alkyl)-(C═O)—NH— group, a (R⁷R⁸N)—(C₁-C₆-alkyl)- group, and a a R⁹OOC— group,
      wherein said heterocycloalkyl group is connected to the rest of the molecule via a carbon atom of said heterocycloalkyl group;
R⁷ and R³ are each independently selected from a hydrogen atom, a C₁-C₆-alkyl group, a C₃-C₃-cycloalkyl group, a C₁-C₆-haloalkyl group, a (C₁-C₆-alkyl)C(═O)— group, a (C₃-C₃-cycloalkyl)-(C₁-C₆-alkyl)- group, a (C₁-C₆-alkoxy)-(C₁-C₆-alkyl)- group, a phenyl group, and a heterocycloalkyl group,
   wherein said heterocycloalkyl group is connected to the rest of the molecule via a carbon atom of said heterocycloalkyl group,
   wherein said phenyl or heterocycloalkyl group is each optionally substituted with one or more substituents independently selected from a halogen atom, a C₁-C₃-alkyl group, a C₁-C₃-haloalkyl group and a C₁-C₃-alkoxy group;
or R⁷ and R^{a} together with the nitrogen atom to which they are attached form a 4- to 7-membered nitrogen-containing heterocycloalkyl group, wherein said heterocycloalkyl group optionally contains one, two or three further heteroatoms independently selected from nitrogen, oxygen and sulfur and is optionally substituted with one or more substituents independently selected from a halogen atom, a C₁-C₃-alkyl group, a C₁-C₃-haloalkyl group, a C₁-C₃-alkoxy group and a phenyl group;
R⁹ is selected from a hydrogen atom, a C₁-C₃-alkyl group and a C₃-C₅-cycloalkyl group;
R^{a} and R^{b} together with the nitrogen atom to which they are attached form a 4-membered nitrogen-containing heterocycloalkyl group which is optionally substituted one, two or three times, each substituent independently selected from a halogen atom, a C₁-C₂-alkyl group, a C₁-C₂-haloalkyl group, a C₁-C₂-alkoxy group, a C₁-C₃-hydroxyalkyl group and a phenyl group;
or a tautomer, or an N-oxide, or a salt thereof, or a salt of a tautomer, or a salt of an N-oxide, or a mixture of same.

### DETAILED DESCRIPTION

### Definitions

The term “substituted” means that one or more hydrogen atoms on the designated atom or group are replaced with a selection from the indicated group, provided that the designated atom's normal valency under the existing circumstances is not exceeded. Combinations of substituents and/or variables are permissible.

The term “optionally substituted” means that the number of substituents can be equal to or different from zero. Unless otherwise indicated, it is possible that optionally substituted groups are substituted with as many optional substituents as can be accommodated by replacing a hydrogen atom with a non-hydrogen substituent on any available carbon or nitrogen atom. Commonly, it is possible for the number of optional substituents, when present, to be 1, 2, 3, 4 or 5, in particular 1, 2 or 3, more particularly 1 or 2, and even more particularly 1.

As used herein, the term “one or more”, e.g., in the definition of the substituents of the compounds of general formula (I) of the present invention, means “1, 2, 3, 4 or 5, particularly 1, 2, 3 or 4, more particularly 1, 2 or 3, even more particularly 1 or 2”. In the context of the present invention, when applied to the substituents of the compounds of general formula (I), the term “one or more” is to be understood as meaning preferably 1, 2 or 3 substituents, and more preferably 1 or 2 substituents.

When groups in the compounds according to the invention are substituted, it is possible for said groups to be mono-substituted or poly-substituted with substituent(s), unless otherwise specified. Within the scope of the present invention, the meanings of all groups which occur repeatedly are independent from one another. It is possible that groups in the compounds according to the invention are substituted with one, two or three identical or different substituents, particularly with one, two or three substituents, more particularly with one substituent.

The terms “oxo”, “an oxo group” or “an oxo substituent” mean a doubly bonded oxygen atom=O. Oxo may be attached to atoms of suitable valency, for example to a saturated carbon atom or to a sulfur atom. For example, but without limitation, one oxo group can be attached to a carbon atom, resulting in the formation of a carbonyl group C(═O), or two oxo groups can be attached to one sulfur atom, resulting in the formation of a sulfonyl group —S(═O)₂.

The term “ring substituent” means a substituent attached to an aromatic or nonaromatic ring which replaces an available hydrogen atom on the ring.

Should a composite substituent be composed of more than one parts, e.g. (C₁-C₄-alkoxy)-(C₁-C₄-alkyl)-, it is possible for the position of a given part to be at any suitable position of said composite substituent, i.e. the C₁-C₄-alkoxy part can be attached to any carbon atom of the C₁-C₄-alkyl part of said (C₁-C₄-alkoxy)-(C₁-C₄-alkyl)- group. A hyphen at the beginning or at the end of such a composite substituent indicates the point of attachment of said composite substituent to the rest of the molecule. Should a ring, comprising carbon atoms and optionally one or more heteroatoms, such as nitrogen, oxygen or sulfur atoms for example, be substituted with a substituent, it is possible for said substituent to be bound at any suitable position of said ring, be it bound to a suitable carbon atom and/or to a suitable heteroatom.

The term “comprising” when used in the specification includes “consisting of”.

If within the present text any item is referred to as “as mentioned herein”, it means that it may be mentioned anywhere in the present text.

If within the present text any item is referred to as “supra” within the description it indicates any of the respective disclosures made within the specification in any of the preceding pages, or above on the same page.

If within the present text any item is referred to as “infra” within the description it indicates any of the respective disclosures made within the specification in any of the subsequent pages, or below on the same page.

The terms as mentioned in the present text have the following meanings:

The term “halogen atom” means a fluorine, chlorine, bromine or iodine atom, particularly a fluorine, chlorine or bromine atom, more particularly a fluorine atom.

The term “C₁-C₆-alkyl” means a linear or branched, saturated, monovalent hydrocarbon group having 1, 2, 3, 4, 5 or 6 carbon atoms, e.g. a methyl-, ethyl-, propyl-, isopropyl-, butyl-, sec-butyl-, isobutyl-, tert-butyl-, pentyl-, isopentyl-, 2-methylbutyl-, 1-methylbutyl-, 1-ethylpropyl-, 1,2-dimethylpropyl-, neo-pentyl-, 1,1-dimethylpropyl-, hexyl-, 1-methylpentyl-, 2-methylpentyl-, 3-methylpentyl-, 4-methylpentyl-, 1-ethylbutyl-, 2-ethylbutyl-, 1,1-dimethylbutyl-, 2,2-dimethylbutyl-, 3,3-dimethylbutyl-, 2,3-dimethylbutyl-, 1,2-dimethylbutyl- or a 1,3-dimethylbutyl- group, or an isomer thereof. Particularly, said group has 1, 2, 3 or 4 carbon atoms (“C₁-C₄-alkyl”), e.g. a methyl-, ethyl-, propyl-, isopropyl-, butyl-, sec-butyl-, isobutyl- or a tert-butyl group, more particularly 1, 2 or 3 carbon atoms (“C₁-C₃-alkyl”), e.g. a methyl-, ethyl-, n-propyl- or an isopropyl group.

The term “C₁-C₆-hydroxyalkyl” means a linear or branched, saturated, monovalent hydrocarbon group in which the term “C₁-C₆-alkyl” is defined supra, and in which one or more hydrogen atoms are replaced with a hydroxy group, e.g. a hydroxymethyl-, 1-hydroxyethyl-, 2-hydroxyethyl-, 1,2-dihydroxyethyl-, 3-hydroxypropyl-, 2-hydroxypropyl-, 1-hydroxypropyl-, 1-hydroxypropan-2-yl-, 2-hydroxypropan-2-yl-, 2,3-dihydroxypropyl-, 1,3-dihydroxypropan-2-yl-, 3-hydroxy-2-methyl-propyl-, 2-hydroxy-2-methyl-propyl- or a 1-hydroxy-2-methyl-propyl- group.

The term “C₁-C₆-alkylsulfanyl” means a linear or branched, saturated, monovalent group of formula (C₁-C₆-alkyl)-S—, in which the term “C₁-C₆-alkyl” is as defined supra, e.g. a methylsulfanyl-, ethylsulfanyl-, propylsulfanyl-, isopropylsulfanyl-, butylsulfanyl-, sec-butylsulfanyl-, isobutylsulfanyl-, tert-butylsulfanyl-, pentylsulfanyl-, isopentylsulfanyl- or a hexylsulfanyl- group.

The term “C₁-C₆-haloalkyl” means a linear or branched, saturated, monovalent hydrocarbon group in which the term “C₁-C₆-alkyl” is as defined supra and in which one or more of the hydrogen atoms are replaced, identically or differently, with a halogen atom. Preferably, said halogen atom is a fluorine atom. Said C₁-C₆-haloalkyl, particularly a C₁-C₃-haloalkyl group is, for example, fluoromethyl-, difluoromethyl-, trifluoromethyl-, 2-fluoroethyl-, 2,2-difluoroethyl-, 2,2,2-trifluoroethyl-, pentafluoroethyl-, 3,3,3-trifluoropropyl- or a 1,3-difluoropropan-2-yl group.

The term “C₁-C₆-alkoxy” means a linear or branched, saturated, monovalent group of formula (C₁-C₆-alkyl)-O—, in which the term “C₁-C₆-alkyl” group is as defined supra, e.g. methoxy-, ethoxy-, n-propoxy-, isopropoxy-, n-butoxy-, sec-butoxy-, isobutoxy-, tert-butoxy-, pentyloxy-, isopentyloxy- or a n-hexyloxy group, or an isomer thereof.

The term “C₁-C₆-haloalkoxy” means a linear or branched, saturated, monovalent C₁-C₆-alkoxy group, as defined supra, in which one or more of the hydrogen atoms is replaced, identically or differently, with a halogen atom. Preferably, said halogen atom in “C₁-C₆-haloalkoxy-” is fluorine, resulting in a group referred herein as “C₁-C₆-fluoroalkoxy-”. Representative C₁-C₆-fluoroalkoxy- groups include, for example, —OCF₃, —OCHF₂, —OCH₂F, —OCF₂CF₃ and —OCH₂CF₃.

The term “C₂-C₆-alkenyl-” means a linear or branched, monovalent hydrocarbon group, which contains one or more double bonds and which has 2, 3, 4, 5 or 6 carbon atoms, preferably 2, 3 or 4 carbon atoms (“C₂-C₄-alkenyl-”) or 2 or 3 carbon atoms (“C₂-C₃-alkenyl-”), it being understood that in the case in which said alkenyl- group contains more than one double bond, then said double bonds may be isolated from, or conjugated with, each other. Representative alkenyl groups include, for example, an ethenyl-, prop-2-enyl-, (E)-prop-1-enyl-, (Z)-prop-1-enyl-, iso-propenyl-, but-3-enyl-, (E)-but-2-enyl-, (Z)-but-2-enyl-, (E)-but-1-enyl-, (Z)-but-1-enyl-, 2-methylprop-2-enyl-, 1-methylprop-2-enyl-, 2-methylprop-1-enyl-, (E)-1-methylprop-1-enyl-, (Z)-1-methylprop-1-enyl-, buta-1,3-dienyl-, pent-4-enyl-, (E)-pent-3-enyl-, (Z)-pent-3-enyl-, (E)-pent-2-enyl-, (Z)-pent-2-enyl-, (E)-pent-1-enyl-, (Z)-pent-1-enyl-, 3-methylbut-3-enyl-, 2-methylbut-3-enyl-, 1-methylbut-3-enyl-, 3-methylbut-2-enyl-, (E)-2-methylbut-2-enyl-, (Z)-2-methylbut-2-enyl-, (E)-1-methylbut-2-enyl-, (Z)-1-methylbut-2-enyl-, (E)-3-methylbut-1-enyl-, (Z)-3-methylbut-1-enyl-, (E)-2-methylbut-1-enyl-, (Z)-2-methylbut-1-enyl-, (E)-1-methylbut-1-enyl-, (Z)-1-methylbut-1-enyl-, 1,1-dimethylprop-2-enyl-, 1-ethylprop-1-enyl-, 1-propylvinyl-, 1-isopropylvinyl-, (E)-3,3-dimethylprop-1-enyl-, (Z)-3,3-dimethylprop-1-enyl-, penta-1,4-dienyl-, hex-5-enyl-, (E)-hex-4-enyl-, (Z)-hex-4-enyl-, (E)-hex-3-enyl-, (Z)-hex-3-enyl-, (E)-hex-2-enyl-, (Z)-hex-2-enyl-, (E)-hex-1-enyl-, (Z)-hex-1-enyl-, 4-methylpent-4-enyl-, 3-methylpent-4-enyl-, 2-methylpent-4-enyl-, 1-methylpent-4-enyl-, 4-methylpent-3-enyl-, (E)-3-methylpent-3-enyl-, (Z)-3-methylpent-3-enyl-, (E)-2-methylpent-3-enyl-, (Z)-2-methylpent-3-enyl-, (E)-1-methylpent-3-enyl-, (Z)-1-methylpent-3-enyl-, (E)-4-methylpent-2-enyl-, (Z)-4-methylpent-2-enyl-, (E)-3-methylpent-2-enyl-, (Z)-3-methylpent-2-enyl-, (E)-2-methylpent-2-enyl-, (Z)-2-methylpent-2-enyl-, (E)-1-methylpent-2-enyl-, (Z)-1-methylpent-2-enyl-, (E)-4-methylpent-1-enyl-, (Z)-4-methylpent-1-enyl-, (E)-3-methylpent-1-enyl-, (Z)-3-methylpent-1-enyl-, (E)-2-methylpent-1-enyl-, (Z)-2-methylpent-1-enyl-, (E)-1-methylpent-1-enyl-, (Z)-1-methylpent-1-enyl-, 3-ethylbut-3-enyl-, 2-ethylbut-3-enyl-, 1-ethylbut-3-enyl-, (E)-3-ethylbut-2-enyl-, (Z)-3-ethylbut-2-enyl-, (E)-2-ethylbut-2-enyl-, (Z)-2-ethylbut-2-enyl-, (E)-1-ethylbut-2-enyl-, (Z)-1-ethylbut-2-enyl-, (E)-3-ethylbut-1-enyl-, (Z)-3-ethylbut-1-enyl-, 2-ethylbut-1-enyl-, (E)-1-ethylbut-1-enyl-, (Z)-1-ethylbut-1-enyl-, 2-propylprop-2-enyl-, 1-propylprop-2-enyl-, 2-isopropylprop-2-enyl-, 1-isopropylprop-2-enyl-, (E)-2-propylprop-1-enyl-, (Z)-2-propylprop-1-enyl-, (E)-1-propylprop-1-enyl-, (Z)-1-propylprop-1-enyl-, (E)-2-isopropylprop-1-enyl-, (Z)-2-isopropylprop-1-enyl-, (E)-1-isopropylprop-1-enyl-, (Z)-1-isopropylprop-1-enyl-, hexa-1,5-dienyl- and a 1-(1,1-dimethylethyl-)ethenyl group. Particularly, said group is an ethenyl- or a prop-2-enyl group.

The same definitions can be applied should the alkenyl group be placed within a chain as a bivalent “C₂-C₆-alkenylene” moiety. All names as mentioned above then will bear a “ene” added to their end, thus e.g., a “pentenyl” becomes a bivalent “pentenylene” group.

The term “C₂-C₆-haloalkenyl-” means a linear or branched hydrocarbon group in which one or more of the hydrogen atoms of a “C₂-C₆-alkenyl-” as defined supra are each replaced, identically or differently, by a halogen atom. Preferably, said halogen atom is fluorine, resulting in a group referred herein as “C₂-C₆-fluoroalkenyl-”. Representative C₂-C₆-fluoroalkenyl- groups include, for example, —CH═CF₂, —CF═CH₂, —CF═CF₂, —C(CH₃)═CF₂, —CH═C(F)—CH₃, —CH₂—CF═CF₂ and —CF₂—CH═CH₂.

The term “C₂-C₆-alkynyl-” means a linear or branched, monovalent hydrocarbon group which contains one or more triple bonds, and which contains 2, 3, 4, 5 or 6 carbon atoms, preferably 2, 3 or 4 carbon atoms (“C₂-C₄-alkynyl-”) or 2 or 3 carbon atoms (“C₂-C₃-alkynyl-”). Representative C₂-C₆-alkynyl- groups include, for example, an ethynyl-, prop-1-ynyl-, prop-2-ynyl-, but-1-ynyl-, but-2-ynyl-, but-3-ynyl-, pent-1-ynyl-, pent-2-ynyl, pent-3-ynyl-, pent-4-ynyl-, hex-1-ynyl-, hex-2-ynyl-, hex-3-ynyl-, hex-4-ynyl-, hex-5-ynyl-, 1-methylprop-2-ynyl-, 2-methylbut-3-ynyl-, 1-methylbut-3-ynyl-, 1-methylbut-2-ynyl-, 3-methylbut-1-ynyl-, 1-ethylprop-2-ynyl-, 3-methylpent-4-ynyl-, 2-methylpent-4-ynyl-, 1-methylpent-4-ynyl-, 2-methylpent-3-ynyl-, 1-methylpent-3-ynyl-, 4-methylpent-2-ynyl-, 1-methylpent-2-ynyl-, 4-methylpent-1-ynyl-, 3-methylpent-1-ynyl-, 2-ethylbut-3-ynyl-, 1-ethylbut-3-ynyl-, 1-ethylbut-2-ynyl-, 1-propylprop-2-ynyl-, 1-isopropylprop-2-ynyl-, 2,2-dimethylbut-3-ynyl-, 1,1-dimethylbut-3-ynyl-, 1,1-dimethylbut-2-ynyl- and a 3,3-dimethylbut-1-ynyl- group. Particularly, said alkynyl- group is an ethynyl-, a prop-1-ynyl- or a prop-2-ynyl group.

The term “C₃-C₃-cycloalkyl” means a saturated, monovalent, mono- or bicyclic hydrocarbon ring which contains 3, 4, 5, 6, 7 or 8 carbon atoms (“C₃-C₈-cycloalkyl”). Said C₃-C₃-cycloalkyl group is for example, a monocyclic hydrocarbon ring, e.g. a cyclopropyl-, cyclobutyl-, cyclopentyl-, cyclohexyl-, cycloheptyl- or cyclooctyl- group, or a bicyclic hydrocarbon ring, e.g. a bicyclo[4.2.0]octyl- or an octahydropentalenyl- group.

The term “C₃-C₃-halocycloalkyl” means a saturated, monovalent, mono- or bicyclic hydrocarbon ring which contains 3, 4, 5, 6, 7 or 8 carbon atoms in which the term “C₃-C₃-cycloalkyl” is as defined supra and in which one or more of the hydrogen atoms of the hydrocarbon ring are replaced, identically or differently, with a halogen atom. Preferably, said halogen atom is a fluorine atom. The “C₃-C₃-cycloalkyl” group as defined supra in which one or more of the hydrogen atoms are replaced, identically or differently, with a halogen atom, preferably a fluorine atom, is for example, a monocyclic hydrocarbon ring, e.g. a cyclopropyl-, cyclobutyl-, cyclopentyl-, cyclohexyl-, cycloheptyl- or cyclooctyl-group, or a bicyclic hydrocarbon ring, e.g. a bicyclo[4.2.0]octyl- or an octahydropentalenyl- group.

The term “C₄-C₃-cycloalkenyl” means a monovalent, mono- or bicyclic hydrocarbon ring which contains 4, 5, 6, 7 or 8 carbon atoms and one double bond. Particularly, said ring contains 4, 5 or 6 carbon atoms (“C₄-C₆-cycloalkenyl”). Said C₄-C₃-cycloalkenyl group is for example, a monocyclic hydrocarbon ring, e.g., a cyclobutenyl-, cyclopentenyl-, cyclohexenyl-, cycloheptenyl- or a cyclooctenyl group, or a bicyclic hydrocarbon ring, e.g., a bicyclo[2.2.1]hept-2-enyl- or a bicyclo[2.2.2]oct-2-enyl group.

The term “C₃-C₃-cycloalkoxy” means a saturated, monovalent, mono- or bicyclic group of formula (C₃-C₃-cycloalkyl)-O—, which contains 3, 4, 5, 6, 7 or 8 carbon atoms, in which the term “C₃-C₃-cycloalkyl” is defined supra, e.g. a cyclopropyloxy-, cyclobutyloxy-, cyclopentyloxy-, cyclohexyloxy-, cycloheptyloxy- or a cyclooctyloxy- group.

If the term “heterocycloalkyl” is used without specifying a number of atoms it is meant to be a “4- to 10-membered heterocycloalkyl-” group, more particularly a 5- to 6-membered heterocycloalkyl group. The terms “4- to 7-membered heterocycloalkyl”, “4- to 6-membered heterocycloalkyl” and “5- to 7-membered heterocycloalkyl” mean a monocyclic, saturated heterocycle with “4, 5, 6 or 7” or, respectively, “4, 5 or 6” or “5, 6 or 7” ring atoms in total, which are saturated or partially unsaturated monocycles, bicycles or polycycles that contain one or two identical or different ring heteroatoms selected from nitrogen, oxygen and sulfur. It is possible for said heterocycloalkyl group to be attached to the rest of the molecule via any one of the carbon atoms or, if present, a nitrogen atom.

Exemplarily, without being limited thereto, said “4- to 7-membered heterocycloalkyl”, can be a 4-membered ring, a “4-membered heterocycloalkyl-” group, such as an azetidinyl- or an oxetanyl group; or a 5-membered ring, a “5-membered heterocycloalkyl-” group, such as a tetrahydrofuranyl-, dioxolinyl-, pyrrolidinyl-, imidazolidinyl-, pyrazolidinyl- or a pyrrolinyl group; or a 6-membered ring, a “6-membered heterocycloalkyl-” group, such as a tetrahydropyranyl-, piperidinyl-, morpholinyl-, 3-oxomorpholin-4-yl, dithianyl-, thiomorpholinyl- or a piperazinyl group; or a 7-membered ring, a “7-membered heterocycloalkyl-” group, such as an azepanyl-, diazepanyl- or an oxazepanyl group, for example. The heterocycloalkyl groups may be substituted one or more times independently with C₁-C₃-alkyl, C₁-C₃-alkoxy, hydroxy, halogen or a carbonyl group.

Particularly, “4- to 6-membered heterocycloalkyl” means a 4- to 6-membered heterocycloalkyl as defined supra containing one ring nitrogen atom and optionally one further ring heteroatom selected from nitrogen, oxygen and sulfur. Particularly, “5- to 7-membered heterocycloalkyl” means a 5- to 7-membered heterocycloalkyl as defined supra containing one ring nitrogen atom and optionally one further ring heteroatom selected from nitrogen, oxygen and sulfur. More particularly, “5- or 6-membered heterocycloalkyl” means a monocyclic, saturated heterocycle with 5 or 6 ring atoms in total, containing one ring nitrogen atom and optionally one further ring heteroatom selected from nitrogen and oxygen.

The term “heteroaryl-” means a monocyclic, bicyclic or tricyclic aromatic ring system having 5, 6, 7, 8, 9, 10, 11, 12, 13 or 14 ring atoms (a “5- to 14-membered heteroaryl-” group), preferably 5, 6, 9 or 10 ring atoms and which contains 1, 2, 3 or 4 heteroatoms which may be identical or different, said heteroatoms being selected from oxygen, nitrogen and sulfur. Said heteroaryl- group can be a 5-membered heteroaryl group, such as, for example, a thienyl-, furanyl-, pyrrolyl-, oxazolyl-, thiazolyl-, imidazolyl-, pyrazolyl-, isoxazolyl-, isothiazolyl-, oxadiazolyl-, triazolyl-, thiadiazolyl- or a tetrazolyl group; or a 6-membered heteroaryl group, such as, for example, a pyridyl-, pyridazinyl-, pyrimidyl-, pyrazinyl- or a triazinyl group; or a benzo-fused 5-membered heteroaryl- group, such as, for example, a benzofuranyl-, benzothienyl-, benzoxazolyl-, benzisoxazolyl-, benzimidazolyl-, benzothiazolyl-, benzotriazolyl-, indazolyl-, indolyl- or a isoindolyl group; or a benzo-fused 6-membered heteroaryl group, such as, for example, a quinolinyl-, quinazolinyl-, isoquinolinyl-, cinnolinyl-, phthalazinyl- or quinoxalinyl-; or another bicyclic group, such as, for example, indolizinyl-, purinyl- or a pteridinyl group.

Preferably, “heteroaryl-” is a monocyclic aromatic ring system having 5 or 6 ring atoms and which contains at least one heteroatom, if more than one, they may be identical or different, said heteroatom being selected from oxygen, nitrogen and sulfur, a (“5- to 6-membered monocyclic heteroaryl-”) group, such as, for example, a thienyl-, furanyl-, pyrrolyl-, oxazolyl-, thiazolyl-, imidazolyl-, pyrazolyl-, isoxazolyl-, isothiazolyl-, oxadiazolyl-, triazolyl-, thiadiazolyl-, tetrazolyl-, pyridyl-, pyridazinyl-, pyrimidyl-, pyrazinyl- or a triazinyl group.

In particular, in the context of the present invention, when applied to any of the substituents of the compounds of general formula (I) such as R², R⁶, R⁷ and/or R⁸, the term “heteroaryl” is to be understood as meaning preferably a monocyclic aromatic ring system having 5 or 6 ring atoms and which contains one or two heteroatoms, which may be identical or different, said heteroatom being selected from oxygen and nitrogen, i.e. a (“5- to 6-membered monocyclic heteroaryl-”) group.

In general, and unless otherwise mentioned, said heteroaryl- groups include all the possible isomeric forms thereof, e.g., the positional isomers thereof. Thus, for some illustrative non-restricting example, the term pyridyl- includes pyridin-2-yl-, pyridin-3-yl- and pyridin-4-yl-; the term thienyl- includes thien-2-yl- and thien-3-yl-, and a heteroarylene group may be inserted into a chain also in the inverse way such as e.g. a 2,3-pyridinylene includes pyridine-2,3-yl as well as pyridine-3,2-yl. Furthermore, said heteroaryl- groups can be attached to the rest of the molecule via any one of the carbon atoms, or, if applicable, a nitrogen atom, e.g., a pyrrol-1-yl-, a pyrazol-1-yl- or an imidazol-1-yl- group.

Particularly, the heteroaryl group is a pyridyl- or pyrimidyl group or a imidazolyl group, including a hydroxy substitution of the pyridyl group leading, e.g., to a 2-hydroxy-pyridine which is the tautomeric form to a 2-oxo-2(1H)-pyridine. In some embodiments, the heteroaryl group is an oxazolyl group.

Further, as used herein, the term “C₃-C₈”, as used throughout this text, e.g., in the context of the definition of “C₃-C₃-cycloalkyl-”, is to be understood as meaning e.g. a cycloalkyl-group having a whole number of carbon atoms of 3 to 8, i.e., 3, 4, 5, 6, 7 or 8 carbon atoms. It is to be understood further that said term “C₃-C₈” is to be interpreted as disclosing any sub-range comprised therein, e.g., C₃-C₆, C₄-C₅, C₃-C₅, C₃-C₄, C₄-C₆, C₅-C₇, preferably C₃-C₆.

Similarly, as used herein, the term “C₂-C₆”, as used throughout this text, e.g., in the context of the definitions of “C₂-C₆-alkenyl-” and “C₂-C₆-alkynyl-”, is to be understood as meaning an alkenyl- group or an alkynyl- group having a whole number of carbon atoms from 2 to 6, i.e., 2, 3, 4, 5 or 6 carbon atoms. It is to be understood further that said term “C₂-C₆” is to be interpreted as disclosing any sub-range comprised therein, e.g., C₂-C₆, C₃-C₅, C₃-C₄, C₂-C₃, C₂-C₄, C₂-C₆ preferably C₂-C₃.

The term “C₁-C₆”, as used throughout this text, e.g., in the context of the definition of “C₁-C₆-alkyl-”, “C₁-C₆-haloalkyl-”, “C₁-C₆-alkoxy-” or “C₁-C₆-haloalkoxy-” is to be understood as meaning an alkyl group having a whole number of carbon atoms from 1 to 6, i.e., 1, 2, 3, 4, 5 or 6 carbon atoms. It is to be understood further that said term “C₁-C₆” is to be interpreted as disclosing any sub-range comprised therein, e.g. C₁-C₆, C₂-C₆, C₃-C₄, C₁-C₂, C₁-C₃, C₁-C₄, C₁-C₅, C₁-C₆; preferably C₁-C₂, C₁-C₃, C₁-C₄, C₁-C₅, C₁-C₆ more preferably C₁-C₄; in the case of “C₁-C₆-haloalkyl-” or “C₁-C₆-haloalkoxy-” even more preferably C₁-C₂.

When a range of values is given, said range encompasses each value and sub-range within said range.

For example:
“C₁-C₆” encompasses C₁, C₂, C₃, C₄, C₅, C₆, C₁-C₆, C₁-C₅, C₁-C₄, C₁-C₃, C₁-C₂, C₂- C₆, C₂-C₅, C₂-C₄, C₂-C₃, C₃-C₆, C₃-C₅, C₃-C₄, C₄-C₆, C₄-C₅, and C₅-C₆;
“C₂-C₆” encompasses C₂, C₃, C₄, C₅, C₆, C₂-C₆, C₂-C₆, C₂-C₄, C₂-C₃, C₃-C₆, C₃-C₅, C₃-C₄, C₄-C₆, C₄-C₅, and C₅-C₆;
“C₃-C₁₀” encompasses C₃, C₄, C₅, C₆, C₇, C₈, C₉, C₁₀, C₃-C₁, C₃-C₉, C₃-C₈, C₃-C₇, C₃-C₆, C₃-C₅, C₃-C₄, C₄-C₁a, C₄-C₉, C₄-C₈, C₄-C₇, C₄-C₆, C₄-C₅, C₅-C₁₀, C₅-C₉, C₅-C₈, C₅-C₇, C₅-C₆, C₆-C₁a, C₆-C₉, C₆-C₈, C₆-C₇, C₇-C₁₀, C₇-C₉, C₇-C₈, C₈-C₁₀, C₈-C₉ and C₉-C₁₀;
“C₃-C₈” encompasses C₃, C₄, C₅, C₆, C₇, C₈, C₃-C₈, C₃-C₇, C₃-C₆, C₃-C₅, C₃-C₄, C₄- C₈, C₄-C₇, C₄-C₆, C₄-C₅, C₅-C₈, C₅-C₇, C₅-C₆, C₆-C₈, C₆-C₇ and C₇-C₈;
“C₃-C₈” encompasses C₃, C₄, C₅, C₆, C₃-C₆, C₃-C₅, C₃-C₄, C₄-C₆, C₄-C₅, and C₅-C₆;
“C₄-C₈” encompasses C₄, C₅, C₆, C₇, C₈, C₄-C₈, C₄-C₇, C₄-C₆, C₄-C₅, C₅-C₈, C₅-C₇, C₅-C₆, C₆-C₈, C₆-C₇ and C₇-C₈;
“C₄-C₈” encompasses C₄, C₅, C₆, C₇, C₄-C₇, C₄-C₆, C₄-C₅, C₅-C₇, C₅-C₆ and C₆-C₇;
“C₄-C₈” encompasses C₄, C₅, C₆, C₄-C₆, C₄-C₅ and C₅-C₆;
“C₅-C₁” encompasses C₅, C₆, C₇, C₈, C₉, C₁, C₅-C₁a, C₅-C₉, C₅-C₈, C₅-C₇, C₅-C₆, C₆- C₁₀, C₆-C₉, C₆-C₈, C₆-C₇, C₇-C₁₀, C₇-C₉, C₇-C₈, C₈-C₁₀, C₈-C₉ and C₉-C₁a;
“C₆-C₁₀” encompasses C₆, C₇, C₈, C₉, C₁a, C₆-C₁₀, C₆-C₉, C₆-C₈, C₆-C₇, C₇-C₁₀, C₇-C₉, C₇-C₈, C₈-C₁₀, C₈-C₉ and C₉-C₁₀.

In particular embodiments for the compounds of formula (I) of the present invention, reference is made to the fact that, when substituted, a particular phenyl group is preferably substituted in one or two of the ortho-positions with respect to the point of attachment of said phenyl group to the rest of the molecule. This is shown below, where the ortho-positions in which the phenyl group is preferably substituted with respect to the point of attachment to the rest of the molecule are marked (*) and where “R” denotes any possible substituents on the phenyl ring, either in the marked and preferred ortho-positions or in any other position(s):

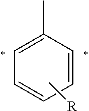

In particular, preferred embodiments of the compounds of formula (I) of the present invention may comprise a phenyl group as substituent R⁶ which is, when substituted, preferably substituted in one or two of the ortho-positions (marked with * below, where “R” denotes any possible substituents of the phenyl ring, either in the marked and preferred ortho-positions or in any other position(s)) with respect to the point of attachment of said phenyl group to the rest of the molecule:

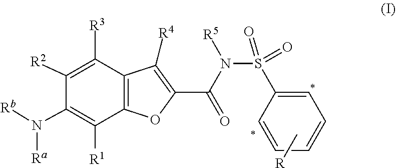

As used herein, the term “leaving group” refers to an atom or a group of atoms that is displaced in a chemical reaction as stable species taking with it the bonding electrons, e.g., typically forming an anion. Preferably, a leaving group is selected from the group comprising: halo, in particular a chloro, bromo or iodo, (methylsulfonyl)oxy-, [(4-methylphenyl)sulfonyl]oxy-, [(trifluoromethyl)sulfonyl]oxy-, [(nonafluorobutyl)sulfonyl]oxy-[(4-bromophenyl)sulfonyl]oxy-, [(4-nitrophenyl)sulfonyl]oxy-, [(2-nitro-phenyl)sulfonyl]oxy-, [(4-isopropylphenyl)sulfonyl]oxy-, [(2,4,6-triisopropylphenyl)sulfonyl]oxy-, [(2,4,6-trimethylphenyl)sulfonyl]oxy-, [(4-tert-butylphenyl)sulfonyl]oxy-, (phenylsulfonyl)oxy-, and a [(4-methoxyphenyl)sulfonyl]oxy group.

As used herein, the term “protective group” is a protective group attached to an oxygen or nitrogen atom in intermediates used for the preparation of compounds of the general formula (I). Such groups are introduced e.g., by chemical modification of the respective hydroxy or amino group in order to obtain chemoselectivity in a subsequent chemical reaction. Protective groups for hydroxy and amino groups are described for example in T. W. Greene and P. G. M. Wuts in Protective Groups in Organic Synthesis, 4^{th} edition, Wiley 2006; more specifically, protective groups for amino groups can be selected from substituted sulfonyl groups, such as a mesyl-, tosyl- or a phenylsulfonyl group, acyl groups such as a benzoyl-, acetyl- or a tetrahydropyranoyl group, or carbamate based groups, such as a tert-butoxycarbonyl group (Boc). Protective groups for hydroxy groups can be selected from acyl groups such as a benzoyl-, acetyl-, pivaloyl- or a tetrahydropyranoyl group, or can include silicon, as in e.g., a tert-butyldimethylsilyl-, tert-butyldiphenylsilyl-, triethylsilyl- or a triisopropylsilyl group.

The term “substituent” refers to a group “substituted” on, e.g., an alkyl-, haloalkyl-, cycloalkyl-, heterocyclyl-, heterocycloalkenyl-, cycloalkenyl-, aryl-, or a heteroaryl group at any atom of that group, replacing one or more hydrogen atoms therein. In one aspect, the substituent(s) on a group are independently any one single, or any combination of two or more of the permissible atoms or groups of atoms delineated for that substituent.

In another aspect, a substituent may itself be substituted with any one of the above substituents. Further, as used herein, the phrase “optionally substituted” means unsubstituted (e.g., substituted with an H) or substituted.

It will be understood that the description of compounds herein is limited by principles of chemical bonding known to those skilled in the art. Accordingly, where a group may be substituted by one or more of a number of substituents, such substitutions are selected so as to comply with principles of chemical bonding with regard to valencies, etc., and to give compounds which are not inherently unstable. For example, any carbon atom will be bonded to two, three, or four other atoms, consistent with the four valence electrons of carbon.

By “subject” is meant a mammal, including, but not limited to, a human or non-human mammal, such as a bovine, equine, canine, ovine, rodent, or feline.

It is possible for the compounds of general formula (I) to exist as isotopic variants. The invention therefore includes one or more isotopic variant(s) of the compounds of general formula (I), particularly deuterium-containing compounds of general formula (I).

The invention also includes all suitable isotopic variations of a compound of the invention.

The term “isotopic variant” of a compound or a reagent is defined as a compound exhibiting an unnatural proportion of one or more of the isotopes that constitute such a compound.

The expression “unnatural proportion” in relation to an isotope means a proportion of such isotope which is higher than its natural abundance. The natural abundances of isotopes to be applied in this context are described in “Isotopic Compositions of the Elements 1997”, Pure Appl. Chem., 70(1), 217-235, 1998.

An isotopic variation of a compound of the invention is defined as one in which at least one atom is replaced by an atom having the same atomic number but an atomic mass different from the atomic mass usually or predominantly found in nature. Examples of isotopes that can be incorporated into a compound of the invention include isotopes of hydrogen, carbon, nitrogen, oxygen, phosphorus, sulphur, fluorine, chlorine, bromine and iodine, such as ²H (deuterium), ³H (tritium), ¹¹C, ¹³C, ¹⁴C, ¹⁵N, ¹⁷O, ¹⁸O, ³²P, ³³P, ³³S, ³⁴S ³⁵S, ³⁶S, ¹⁸F, ³⁶Cl, ⁸²Br, ¹²³I, ¹²⁴I, ¹²⁹I and ¹³¹I, respectively. Accordingly, recitation of “hydrogen” or “H” should be understood to encompass ¹H (protium), ²H (deuterium), and ³H (tritium) unless otherwise specified. Certain isotopic variations of a compound of the invention, for example, those in which one or more radioactive isotopes such as ³H or ¹⁴C are incorporated, are useful in drug and/or substrate tissue distribution studies. Tritiated and carbon-14, i.e., ¹⁴C, isotopes are particularly preferred for their ease of preparation and detectability. Further, substitution with isotopes such as deuterium may afford certain therapeutic advantages resulting from greater metabolic stability, for example, increased in vivo half-life or reduced dosage requirements and hence may be preferred in some circumstances. Isotopic variations of a compound of the invention can generally be prepared by conventional procedures known by a person skilled in the art such as by the illustrative methods or by the preparations described in the examples hereafter using appropriate isotopic variations of suitable reagents.

With respect to the treatment and/or prophylaxis of the disorders specified herein, the isotopic variant(s) of the compounds of general formula (I) preferably contain deuterium (“deuterium-containing compounds of general formula (I)”). Isotopic variants of the compounds of general formula (I) in which one or more radioactive isotopes, such as ³H or ¹⁴C, are incorporated are useful, e.g., in drug and/or substrate tissue distribution studies. These isotopes are particularly preferred for the ease of their incorporation and detectability. Positron-emitting isotopes such as ¹⁸F or ¹¹C may be incorporated into a compound of general formula (I). These isotopic variants of the compounds of general formula (I) are useful for in vivo imaging applications. Deuterium-containing and ¹³C containing compounds of general formula (I) can be used in mass spectrometry analyses in the context of preclinical or clinical studies.

Isotopic variants of the compounds of general formula (I) can generally be prepared by methods known to a person skilled in the art, such as those described in the schemes and/or examples herein, by substituting a reagent for an isotopic variant of said reagent, preferably for a deuterium-containing reagent. Depending on the desired sites of deuteration, in some cases deuterium from D₂O can be incorporated either directly into the compounds or into reagents that are useful for synthesizing such compounds. Deuterium gas is also a useful reagent for incorporating deuterium into molecules. Catalytic deuteration of olefinic bonds and acetylenic bonds is a rapid route for incorporation of deuterium. Metal catalysts (i.e. Pd, Pt, and Rh) in the presence of deuterium gas can be used to directly exchange deuterium for hydrogen in functional groups containing hydrocarbons. A variety of deuterated reagents and synthetic building blocks are commercially available from companies such as for example C/D/N Isotopes, Quebec, Canada; Cambridge Isotope Laboratories Inc., Andover, MA, USA; and CombiPhos Catalysts, Inc., Princeton, NJ, USA.

The term “deuterium-containing compound of general formula (I)” is defined as a compound of general formula (I), in which one or more hydrogen atom(s) is/are replaced by one or more deuterium atom(s) and in which the abundance of deuterium at each deuterated position of the compound of general formula (I) is higher than the natural abundance of deuterium, which is about 0.015%. Particularly, in a deuterium-containing compound of general formula (I) the abundance of deuterium at each deuterated position of the compound of general formula (I) is higher than 10%, 20%, 30%, 40%, 50%, 60%, 70% or 80%, preferably higher than 90%, 95%, 96% or 97%, even more preferably higher than 98% or 99% at said position(s). It is understood that the abundance of deuterium at each deuterated position is independent of the abundance of deuterium at other deuterated position(s).

The selective incorporation of one or more deuterium atom(s) into a compound of general formula (I) may alter the physicochemical properties (such as for example acidity [C. L. Perrin, et al., J. Am. Chem. Soc., 2007, 129, 4490], basicity [C. L. Perrin et al., J. Am. Chem. Soc., 2005, 127, 9641], lipophilicity [B. Testa et al., Int. J. Pharm., 1984, 19(3), 271]) and/or the metabolic profile of the molecule and may result in changes in the ratio of parent compound to metabolites or in the amounts of metabolites formed. Such changes may result in certain therapeutic advantages and hence may be preferred in some circumstances. Reduced rates of metabolism and metabolic switching, where the ratio of metabolites is changed, have been reported (A. E. Mutlib et al., Toxicol. Appl. Pharmacol., 2000, 169, 102). These changes in the exposure to parent drug and metabolites can have important consequences with respect to the pharmacodynamics, tolerability and efficacy of a deuterium-containing compound of general formula (I). In some cases deuterium substitution reduces or eliminates the formation of an undesired or toxic metabolite and enhances the formation of a desired metabolite (e.g., Nevirapine: A. M. Sharma et al., Chem. Res. Toxicol., 2013, 26, 410; Efavirenz: A. E. Mutlib et al., Toxicol. Appl. Pharmacol., 2000, 169, 102). In other cases the major effect of deuteration is to reduce the rate of systemic clearance. As a result, the biological half-life of the compound is increased. The potential clinical benefits would include the ability to maintain similar systemic exposure with decreased peak levels and increased trough levels. This could result in lower side effects and enhanced efficacy, depending on the particular compound's pharmacokinetic/pharmacodynamic relationship. ML-337 (C. J. Wenthur et al., J. Med. Chem., 2013, 56, 5208) and Odanacatib (K. Kassahun et al., WO2012/112363) are examples for this deuterium effect. Still other cases have been reported in which reduced rates of metabolism result in an increase in exposure of the drug without changing the rate of systemic clearance (e.g., Rofecoxib: F. Schneider et al., Arzneim. Forsch./Drug. Res., 2006, 56, 295; Telaprevir: F. Maltais et al., J. Med. Chem., 2009, 52, 7993). Deuterated drugs showing this effect may have reduced dosing requirements (e.g., lower number of doses or lower dosage to achieve the desired effect) and/or may produce lower metabolite loads.

A compound of general formula (I) may have multiple potential sites of attack for metabolism. To optimize the above-described effects on physicochemical properties and metabolic profile, deuterium-containing compounds of general formula (I) having a certain pattern of one or more deuterium-hydrogen exchange(s) can be selected. Particularly, the deuterium atom(s) of deuterium-containing compound(s) of general formula (I) is/are attached to a carbon atom and/or is/are located at those positions of the compound of general formula (I), which are sites of attack for metabolizing enzymes such as e.g. cytochrome P₄₅₀.

Where the plural form of the word compounds, salts, polymorphs, hydrates, solvates and the like, is used herein, this is taken to mean also a single compound, salt, polymorph, isomer, hydrate, solvate or the like.

By “stable compound” or “stable structure” is meant a compound that is sufficiently robust to survive isolation to a useful degree of purity from a reaction mixture, and formulation into an efficacious therapeutic agent.

The compounds of the present invention optionally contain one or more asymmetric centres, depending upon the location and nature of the various substituents desired. It is possible that one or more asymmetric carbon atoms are present in the (R) or (S) configuration, which can result in racemic mixtures in the case of a single asymmetric centre, and in diastereomeric mixtures in the case of multiple asymmetric centres. In certain instances, it is possible that asymmetry also be present due to restricted rotation about a given bond, for example, the central bond adjoining two substituted aromatic rings of the specified compounds.

Preferred compounds are those which produce the more desirable biological activity. Separated, pure or partially purified isomers and stereoisomers or racemic or diastereomeric mixtures of the compounds of the present invention are also included within the scope of the present invention. The purification and the separation of such materials can be accomplished by standard techniques known in the art.

Preferred isomers are those which produce the more desirable biological activity. These separated, pure or partially purified isomers or racemic mixtures of the compounds of this invention are also included within the scope of the present invention. The purification and the separation of such materials can be accomplished by standard techniques known in the art.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (e.g., HPLC columns using a chiral phase), with or without conventional derivatisation, optimally chosen to maximise the separation of the enantiomers. Suitable HPLC columns using a chiral phase are commercially available, such as those manufactured by Daicel, e.g., Chiracel OD and Chiracel OJ, for example, among many others, which are all routinely selectable. Enzymatic separations, with or without derivatisation, are also useful. The optically active compounds of the present invention can likewise be obtained by chiral syntheses utilizing optically active starting materials.

In order to distinguish different types of isomers from each other reference is made to IUPAC Rules Section E (Pure Appl Chem 45, 11-30, 1976).

The present invention includes all possible stereoisomers of the compounds of the present invention as single stereoisomers, or as any mixture of said stereoisomers, e.g. (R)- or (S)-isomers, in any ratio. Isolation of a single stereoisomer, e.g. a single enantiomer or a single diastereomer, of a compound of the present invention is achieved by any suitable state of the art method, such as chromatography, especially chiral chromatography, for example.

Further, it may be possible for the compounds of the present invention to exist as tautomers. For example, any compound of the present invention which contains an imidazopyridine moiety as a heteroaryl group for example can exist as a 1H tautomer, or a 3H tautomer, or even a mixture in any amount of the two tautomers, namely:

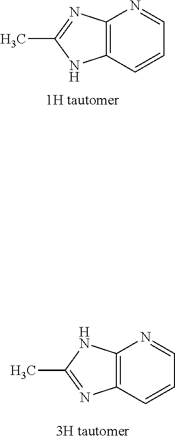

Further, in the context of the present invention, it may be possible for the compounds of formula (I) to exist as tautomers. For example, as depicted below, the compounds of formula (I) according to the present invention can exist as tautomers of formula (I′) and/or formula (I″) or as mixtures in any amount of two or more of the possible tautomers.

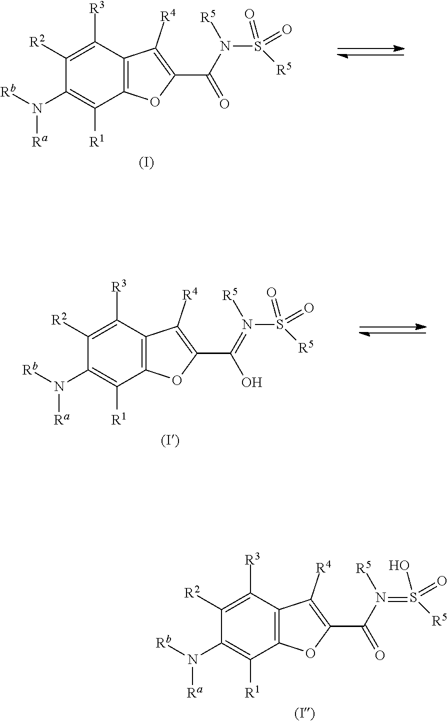

The present invention includes all possible tautomers of the compounds of the present invention as single tautomers, or as any mixture of two or more of any possible tautomers, in any ratio.

Further, the compounds of the present invention can exist as N-oxides, which are defined in that at least one nitrogen of the compounds of the present invention is oxidised. The present invention includes all such possible N-oxides.

The present invention also provides useful forms of the compounds of the present invention, such as metabolites, hydrates, solvates, prodrugs, salts, in particular pharmaceutically acceptable salts, and/or co-precipitates.

The compounds of the present invention can exist as a hydrate, or as a solvate, wherein the compounds of the present invention contain polar solvents, in particular water, methanol or ethanol for example, as structural element of the crystal lattice of the compounds. It is possible for the amount of polar solvents, in particular water, to exist in a stoichiometric or non-stoichiometric ratio. In the case of stoichiometric solvates, e.g. a hydrate, hemi-, (semi-), mono-, sesqui-, di-, tri-, tetra-, penta- etc. solvates or hydrates, respectively, are possible. The present invention includes all such hydrates or solvates.

Further, it is possible for the compounds of the present invention to exist in free form, e.g. as a free base, or as a free acid, or as a zwitterion, or to exist in the form of a salt. Said salt may be any salt, either an organic or inorganic addition salt, particularly any pharmaceutically acceptable organic or inorganic addition salt, which is customarily used in pharmacy, or which is used, for example, for isolating or purifying the compounds of the present invention.

The term “pharmaceutically acceptable salt” refers to an inorganic or organic acid addition salt of a compound of the present invention. For example, see S. M. Berge, et al. “Pharmaceutical Salts,” J. Pharm. Sci. 1977, 66, 1-19.

Physiologically acceptable salts of the compounds according to the invention encompass acid addition salts of mineral acids, carboxylic acids and sulfonic acids, for example salts of hydrochloric acid, hydrobromic acid, hydroiodic acid, sulfuric acid, bisulfuric acid, phosphoric acid, nitric acid or with an organic acid, such as formic, acetic, acetoacetic, pyruvic, trifluoroacetic, propionic, butyric, hexanoic, heptanoic, undecanoic, lauric, benzoic, salicylic, 2-(4-hydroxybenzoyl)-benzoic, camphoric, cinnamic, cyclopentanepropionic, digluconic, 3-hydroxy-2-naphthoic, nicotinic, pamoic, pectinic, persulfuric, 3-phenylpropionic, picric, pivalic, 2-hydroxyethanesulfonate, itaconic, sulfamic, trifluoromethanesulfonic, dodecylsulfuric, ethansulfonic, benzenesulfonic, para-toluenesulfonic, methansulfonic, 2-naphthalenesulfonic, naphthalenedisulfonic, camphorsulfonic acid, citric, tartaric, stearic, lactic, oxalic, malonic, succinic, malic, adipic, alginic, maleic, fumaric, D-gluconic, mandelic, ascorbic, glucoheptanoic, glycerophosphoric, aspartic, sulfosalicylic, hemisulfuric, or thiocyanic acid, for example.

A “pharmaceutically acceptable anion” refers to the deprotonated form of a conventional acid, such as, for example, a hydroxide, a carboxylate, a sulfate, a halide, a phosphate, or a nitrate.

Physiologically acceptable salts of the compounds according to the invention also comprise salts of conventional bases, such as, by way of example and by preference, alkali metal salts (for example lithium, sodium and potassium salts), alkaline earth metal salts (for example calcium, strontium and magnesium salts) and ammonium salts derived from ammonia or organic amines with 1 to 16 C atoms, such as, by way of example and by preference, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine, N-methylpiperidine, N-methylglucamine, dimethylglucamine, ethylglucamine, 1,6-hexadiamine, glucosamine, sarcosine, serinol, tris(hydroxymethyl)aminomethane, aminopropanediol, Sovak base, and 1-amino-2,3,4-butanetriol.

Additionally, the compounds according to the invention may form salts with a quaternary ammonium ion obtainable, e.g., by quaternisation of a basic nitrogen-containing group with agents such as lower alkylhalides such as methyl-, ethyl-, propyl-, and butylchlorides, -bromides and -iodides; dialkylsulfates such as dimethyl-, diethyl-, dibutyl- and diamylsulfates, long chain halides such as decyl-, lauryl-, myristyl- and stearylchlorides, -bromides and -iodides, aralkylhalides such as benzyl- and phenethylbromides and others. Examples of suitable quaternary ammonium ions are tetramethylammonium, tetraethylammonium, tetra(n-propyl)ammonium, tetra (n-butyl)ammonium, or N-benzyl-N,N,N-trimethylammonium.

The present invention includes all possible salts of the compounds of the present invention as single salts, or as any mixture of said salts, in any ratio.

In the present text, in particular in the Experimental Section, for the synthesis of intermediates and of examples of the present invention, when a compound is mentioned as a salt form with the corresponding base or acid, the exact stoichiometric composition of said salt form, as obtained by the respective preparation and/or purification process, is, in most cases, unknown.

Unless specified otherwise, suffixes to chemical names or structural formulae relating to salts, such as “hydrochloride”, “trifluoroacetate”, “sodium salt”, or “x HCl”, “x CF₃COOH”, “x Na⁺”, for example, mean a salt form, the stoichiometry of which salt form not being specified.

This applies analogously to cases in which synthesis intermediates or example compounds or salts thereof have been obtained, by the preparation and/or purification processes described, as solvates, such as hydrates, with (if defined) unknown stoichiometric composition.

Unless specified otherwise, suffixes to chemical names or structural formulae relating to salts, such as “hydrochloride”, “trifluoroacetate”, “sodium salt”, or “x HCl”, “x CF₃COOH”, “x Na⁺”, for example, mean a salt form, the stoichiometry of which salt form not being specified.

Solvates and hydrates of disclosed intermediates or example compounds, or salts thereof, which have been obtained, by the preparation and/or purification processes described herein, may be formed in any ratio.

Furthermore, the present invention includes all possible crystalline forms, or polymorphs, of the compounds of the present invention, either as a single polymorph, or as a mixture of more than one polymorph, in any ratio.

Moreover, the present invention also includes prodrugs of the compounds according to the invention. The term “prodrugs” designates compounds which themselves can be biologically active or inactive, but are converted (for example metabolically or hydrolytically) into compounds according to the invention during their residence time in the body. For example, a prodrug may be in the form of an in vivo hydrolysable ester of the specified compound. Derivatives of the compounds of formula (I) and the salts thereof which are converted into a compound of formula (I) or a salt thereof in a biological system (bioprecursors or pro-drugs) are covered by the invention. Said biological system may be, for example, a mammalian organism, particularly a human subject. The bioprecursor is, for example, converted into the compound of formula (I) or a salt thereof by metabolic processes.

### DESCRIPTION

### Further Embodiments of the First Aspect of the Present Invention

In accordance with further embodiments, the present invention provides compounds of general formula (I), supra, wherein:
R¹ is a hydrogen atom;
R² is a hydrogen atom;
R³ is selected from a hydrogen atom and a fluorine atom;
R⁴ is a hydrogen atom;
R⁵ is a hydrogen atom;
R⁶ is selected from a phenyl group, a naphthyl group and a heteroaryl group,
   wherein said phenyl, naphthyl or heteroaryl group is each optionally substituted with one or more substituents independently selected from a halogen atom, a hydroxy group, a C₁-C₆-alkyl group, a C₁-C₆-hydroxyalkyl group, a C₁-C₆-haloalkyl group, a C₁-C₆-alkoxy group, a (C₁-C₃-alkyl)-O—(C₁-C₂-alkyl)- group, a (C₁-C₃-alkyl)-O—(C₁-C₂-alkyl)-O— group, a (C₃-C₃-cycloalkyl)-(C₁-C₂-alkyl)-O— group, a (C₃-C₃-halocycloalkyl)-(C₁-C₂-alkyl)-O— group, a (phenyl)-(C₁-C₂-alkyl)-O— group, a C₁-C₆-haloalkoxy group, a C₂-C₆-alkenyl group, a C₃-C₃-cycloalkyl group, a C₃-C₃-cycloalkoxy group, a C₁-C₆-thioalkyl group, a heterocycloalkyl group, a R⁷R⁸N— group, a (C₁-C₂-alkyl)-S(═O)₂— group, a (phenyl)-S(═O)₂— group, a (R⁷R⁸N)—S(═O)₂— group, a (H₃C)—(C═O)— group, a (R⁷R⁸N)—(C═O)— group, a (R⁷R⁸N)—(C═O)—O— group, a R⁹OOC—(C₁-C₃-alkyl)- group, a R⁹OOC— group, a phenyl group, a (phenyl)-O— group and a heteroaryl group,
      wherein said heterocycloalkyl group is connected to the rest of the molecule via a carbon atom of said heterocycloalkyl group,
      wherein said phenyl, heteroaryl or heterocycloalkyl group is each optionally substituted with one or more substituents independently selected from a halogen atom, a C₁-C₃-alkyl group, a phenyl group and a R⁹OOC— group;
R⁷ and R³ are each independently selected from a hydrogen atom, a C₁-C₆-alkyl group, a C₃-C₃-cycloalkyl group and a phenyl group,
   wherein said phenyl group is optionally substituted with one or more substituents independently selected from a halogen atom, a C₁-C₃-alkyl group, a C₁-C₃-haloalkyl group and a C₁-C₃-alkoxy group;
or R⁷ and R^{a} together with the nitrogen atom to which they are attached form a 4- to 7-membered nitrogen-containing heterocycloalkyl group, wherein said heterocycloalkyl group optionally contains one, two or three further heteroatoms independently selected from nitrogen, oxygen and sulfur and is optionally substituted with one or more substituents independently selected from a halogen atom, a C₁-C₃-alkyl group, a C₁-C₃-haloalkyl group, a C₁-C₃-alkoxy group and a phenyl group;
R⁹ is selected from a hydrogen atom, a C₁-C₃-alkyl group and a C₃-C₆-cycloalkyl group;
R^{a} and R^{b} together with the nitrogen atom to which they are attached form a 4-membered nitrogen-containing heterocycloalkyl group which is optionally substituted one, two or three times, each substituent independently selected from a halogen atom, a C₁-C₂-alkyl group, a C₁-C₂-haloalkyl group, a C₁-C₂-alkoxy group, a C₁-C₃-hydroxyalkyl group and a phenyl group;
or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is a hydrogen atom;
R² is a hydrogen atom;
R³ is selected from a hydrogen atom and a fluorine atom;
R⁴ is a hydrogen atom;
R⁵ is a hydrogen atom;
R⁶ is selected from a phenyl group, a naphthyl group and a heteroaryl group,
   wherein said phenyl, naphthyl or heteroaryl group is each optionally substituted with one or more substituents independently selected from a halogen atom, a hydroxy group, a C₁-C₆-alkyl group, a C₁-C₆-hydroxyalkyl group, a C₁-C₆-haloalkyl group, a C₁-C₆-alkoxy group, a (C₁-C₃-alkyl)-O—(C₁-C₂-alkyl)- group, a (C₁-C₃-alkyl)-O—(C₁-C₂-alkyl)-O— group, a (C₃-C₃-cycloalkyl)-(C₁-C₂-alkyl)-O— group, a (C₃-C₃-halocycloalkyl)-(C₁-C₂-alkyl)-O— group, a (phenyl)-(C₁-C₂-alkyl)-O— group, a C₁-C₆-haloalkoxy group, a C₂-C₆-alkenyl group, a C₃-C₃-cycloalkyl group, a C₃-C₃-cycloalkoxy group, a C₁-C₆-thioalkyl group, a heterocycloalkyl group, a R⁷R⁸N— group, a (C₁-C₂-alkyl)-S(═O)₂— group, a (H₃C)—(C═O)— group, a (R⁷R⁸N)—(C═O)— group, a (R⁷R⁸N)—(C═O)—O— group, a R⁹OOC—(C₁-C₃-alkyl)- group, a R⁹OOC— group, a phenyl group, a (phenyl)-O— group and a heteroaryl group,
      wherein said heterocycloalkyl group is connected to the rest of the molecule via a carbon atom of said heterocycloalkyl group,
      wherein said phenyl, heteroaryl or heterocycloalkyl group is each optionally substituted with one or more substituents independently selected from a halogen atom and a C₁-C₃-alkyl group;
R⁷ and R³ are each independently selected from a hydrogen atom, a C₁-C₆-alkyl group, a C₃-C₃-cycloalkyl group and a phenyl group,
   wherein said phenyl group is optionally substituted with one or more substituents independently selected from a halogen atom, a C₁-C₃-alkyl group, a C₁-C₃-haloalkyl group and a C₁-C₃-alkoxy group;
or R⁷ and R⁸ together with the nitrogen atom to which they are attached form a 4- to 7-membered nitrogen-containing heterocycloalkyl group, wherein said heterocycloalkyl group optionally contains one, two or three further heteroatoms independently selected from nitrogen, oxygen and sulfur and is optionally substituted with one or more substituents independently selected from a halogen atom, a C₁-C₃-alkyl group, a C₁-C₃-haloalkyl group, a C₁-C₃-alkoxy group and a phenyl group;
R⁹ is selected from a hydrogen atom, a C₁-C₃-alkyl group and a C₃-C₅-cycloalkyl group;
R^{a} and R^{b} together with the nitrogen atom to which they are attached form a 4-membered nitrogen-containing heterocycloalkyl group which is optionally substituted one, two or three times, each substituent independently selected from a halogen atom, a C₁-C₂-alkyl group, a C₁-C₂-haloalkyl group, a C₁-C₂-alkoxy group, a C₁-C₃-hydroxyalkyl group and a phenyl group;
or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is a hydrogen atom;
R² is a hydrogen atom;
R³ is selected from a hydrogen atom and a fluorine atom;
R⁴ is a hydrogen atom;
R⁵ is a hydrogen atom;
R⁶ is selected from a phenyl group, a naphthyl group and a heteroaryl group,
   wherein said phenyl, naphthyl or heteroaryl group is each optionally substituted with one or more substituents independently selected from a halogen atom, a C₁-C₆-alkyl group, a C₁-C₆-haloalkyl group, a C₁-C₆-alkoxy group, a (C₁-C₃-alkyl)-O—(C₁-C₂-alkyl)- group, a (C₃-C₃-cycloalkyl)-(C₁-C₂-alkyl)-O— group, a (C₃-C₃-halocycloalkyl)-(C₁-C₂-alkyl)-O— group, a (phenyl)-(C₁-C₂-alkyl)-O— group, a C₁-C₆-haloalkoxy group, a C₂-C₆-alkenyl group, a C₃-C₃-cycloalkoxy group, a C₁-C₆-thioalkyl group, a heterocycloalkyl group, a R⁷R⁸N— group, a (C₁-C₂-alkyl)-S(═O)₂— group, a (R⁷R⁸N)—(C═O)— group, a (R⁷R⁸N)—(C═O)—O— group, a R⁹OOC—(C₁-C₃-alkyl)- group, a R⁹OOC— group, a phenyl group, a (phenyl)-O— group and a heteroaryl group,
      wherein said heterocycloalkyl group is connected to the rest of the molecule via a carbon atom of said heterocycloalkyl group,
      wherein said phenyl or heteroaryl group is each optionally substituted with one or more substituents independently selected from a halogen atom and a C₁-C₃-alkyl group;
R⁷ and R³ are each independently selected from a hydrogen atom, a C₁-C₆-alkyl group, a C₃-C₃-cycloalkyl group and a phenyl group,
   wherein said phenyl group is optionally substituted with one or more substituents independently selected from a halogen atom, a C₁-C₃-alkyl group, a C₁-C₃-haloalkyl group and a C₁-C₃-alkoxy group;
or R⁷ and R^{a} together with the nitrogen atom to which they are attached form a 4- to 7-membered nitrogen-containing heterocycloalkyl group, wherein said heterocycloalkyl group optionally contains one, two or three further heteroatoms independently selected from nitrogen, oxygen and sulfur and is optionally substituted with one or more substituents independently selected from a halogen atom, a C₁-C₃-alkyl group, a C₁-C₃-haloalkyl group, a C₁-C₃-alkoxy group and a phenyl group;
R⁹ is selected from a hydrogen atom, a C₁-C₃-alkyl group and a C₃-C₆-cycloalkyl group;
R^{a} and R^{b} together with the nitrogen atom to which they are attached form a 4-membered nitrogen-containing heterocycloalkyl group which is optionally substituted one, two or three times, each substituent independently selected from a halogen atom, a C₁-C₂-alkyl group, a C₁-C₂-haloalkyl group, a C₁-C₂-alkoxy group, a C₁-C₃-hydroxyalkyl group and a phenyl group;
or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is a hydrogen atom;
R² is a hydrogen atom;
R³ is selected from a hydrogen atom and a fluorine atom;
R⁴ is a hydrogen atom;
R⁵ is a hydrogen atom;
R⁶ is selected from a phenyl group and a heteroaryl group,
   wherein said phenyl or heteroaryl group is each optionally substituted with one or more substituents independently selected from a halogen atom, a C₁-C₆-alkyl group, a C₁-C₆-haloalkyl group, a C₁-C₆-alkoxy group, a (C₃-C₃-cycloalkyl)-(C₁-C₂-alkyl)-O— group, a (C₃-C₃-halocycloalkyl)-(C₁-C₂-alkyl)-O— group, a (phenyl)-(C₁-C₂-alkyl)-O— group, a C₁-C₆-haloalkoxy group, a C₃-C₃-cycloalkoxy group, a heterocycloalkyl group, a R⁷R⁸N— group, a (phenyl)-O— group and a heteroaryl group,
      wherein said heterocycloalkyl group is connected to the rest of the molecule via a carbon atom of said heterocycloalkyl group,
      wherein said heteroaryl or heterocycloalkyl group is each optionally substituted with one or more substituents independently selected from a C₁-C₃-alkyl group and a phenyl group;
R⁷ and R⁸ are each independently selected from a hydrogen atom, a C₁-C₆-alkyl group, a C₃-C₃-cycloalkyl group and a phenyl group,
   wherein said phenyl group is optionally substituted with one or more substituents independently selected from a halogen atom, a C₁-C₃-alkyl group, a C₁-C₃-haloalkyl group and a C₁-C₃-alkoxy group;
or R⁷ and R^{a} together with the nitrogen atom to which they are attached form a 4- to 7-membered nitrogen-containing heterocycloalkyl group, wherein said heterocycloalkyl group optionally contains one, two or three further heteroatoms independently selected from nitrogen, oxygen and sulfur and is optionally substituted with one or more substituents independently selected from a halogen atom, a C₁-C₃-alkyl group, a C₁-C₃-haloalkyl group, a C₁-C₃-alkoxy group and a phenyl group;
R⁹ is selected from a hydrogen atom, a C₁-C₃-alkyl group and a C₃-C₅-cycloalkyl group;
R^{a} and R^{b} together with the nitrogen atom to which they are attached form a 4-membered nitrogen-containing heterocycloalkyl group which is optionally substituted one, two or three times, each substituent independently selected from a halogen atom, a C₁-C₂-alkyl group, a C₁-C₂-haloalkyl group, a C₁-C₂-alkoxy group, a C₁-C₃-hydroxyalkyl group and a phenyl group;
or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is a hydrogen atom;
R² is a hydrogen atom;
R³ is selected from a hydrogen atom and a fluorine atom;
R⁴ is a hydrogen atom;
R⁵ is a hydrogen atom;
R⁶ is selected from a phenyl group and a heteroaryl group,
   wherein said phenyl or heteroaryl group is each optionally substituted with one or more substituents independently selected from a halogen atom, a C₁-C₆-alkyl group, a C₁-C₆-alkoxy group, a (phenyl)-(C₁-C₂-alkyl)-O— group, a C₁-C₆-haloalkoxy group, a C₂-C₆-alkenyl group, a C₃-C₃-cycloalkoxy group, a heterocycloalkyl group, a phenyl group,
      wherein said heterocycloalkyl group is connected to the rest of the molecule via a carbon atom of said heterocycloalkyl group,
      wherein said phenyl, or heterocycloalkyl group is each optionally substituted with one or more substituents independently selected from a halogen atom, a C₁-C₃-alkyl group and a R⁹OOC— group;
R⁷ and R³ are each independently selected from a hydrogen atom, a C₁-C₆-alkyl group, a C₃-C₃-cycloalkyl group and a phenyl group,
   wherein said phenyl group is optionally substituted with one or more substituents independently selected from a halogen atom, a C₁-C₃-alkyl group, a C₁-C₃-haloalkyl group and a C₁-C₃-alkoxy group;
or R⁷ and R⁸ together with the nitrogen atom to which they are attached form a 4- to 7-membered nitrogen-containing heterocycloalkyl group, wherein said heterocycloalkyl group optionally contains one, two or three further heteroatoms independently selected from nitrogen, oxygen and sulfur and is optionally substituted with one or more substituents independently selected from a halogen atom, a C₁-C₃-alkyl group, a C₁-C₃-haloalkyl group, a C₁-C₃-alkoxy group and a phenyl group;
R⁹ is selected from a hydrogen atom, a C₁-C₃-alkyl group and a C₃-C₆-cycloalkyl group;
R^{a} and R^{b} together with the nitrogen atom to which they are attached form a 4-membered nitrogen-containing heterocycloalkyl group which is optionally substituted one, two or three times, each substituent independently selected from a halogen atom, a C₁-C₂-alkyl group, a C₁-C₂-haloalkyl group, a C₁-C₂-alkoxy group, a C₁-C₃-hydroxyalkyl group and a phenyl group;
or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is a hydrogen atom;
R² is a hydrogen atom;
R³ is selected from a hydrogen atom and a fluorine atom;
R⁴ is a hydrogen atom;
R⁵ is a hydrogen atom;
R⁶ is selected from a phenyl group, a naphthyl group and a heteroaryl group,
   wherein said phenyl, naphthyl or heteroaryl group is each optionally substituted with one or more substituents independently selected from a halogen atom, a C₁-C₆-alkyl group, a C₁-C₆-haloalkyl group, a C₁-C₆-alkoxy group, a (C₁-C₃-alkyl)-O—(C₁-C₂-alkyl)- group, a (C₁-C₃-alkyl)-O—(C₁-C₂-alkyl)-O— group, a C₂-C₆-alkenyl group, a C₁-C₆-thioalkyl group, a heterocycloalkyl group, a R⁷R⁸N— group, a (C₁-C₂-alkyl)-S(═O)₂— group, a (R⁷R⁸N)—(C═O)— group, a (R⁷R⁸N)—(C═O)—O— group, a R⁹OOC—(C₁-C₃-alkyl)- group, a R⁹OOC— group, and a heteroaryl group,
      wherein said heterocycloalkyl group is connected to the rest of the molecule via a carbon atom of said heterocycloalkyl group,
      wherein said heteroaryl group is each optionally substituted with one or more substituents independently selected from a C₁-C₆-alkyl group;
R⁷ and R³ are each independently selected from a hydrogen atom, a C₁-C₆-alkyl group, a C₃-C₃-cycloalkyl group and a phenyl group,
   wherein said phenyl group is optionally substituted with one or more substituents independently selected from a halogen atom, a C₁-C₃-alkyl group, a C₁-C₃-haloalkyl group and a C₁-C₃-alkoxy group;
or R⁷ and R^{a} together with the nitrogen atom to which they are attached form a 4- to 7-membered nitrogen-containing heterocycloalkyl group, wherein said heterocycloalkyl group optionally contains one, two or three further heteroatoms independently selected from nitrogen, oxygen and sulfur and is optionally substituted with one or more substituents independently selected from a halogen atom, a C₁-C₃-alkyl group, a C₁-C₃-haloalkyl group, a C₁-C₃-alkoxy group and a phenyl group;
R⁹ is selected from a hydrogen atom, a C₁-C₃-alkyl group and a C₃-C₆-cycloalkyl group;
R^{a} and R^{b} together with the nitrogen atom to which they are attached form a 4-membered nitrogen-containing heterocycloalkyl group which is optionally substituted one, two or three times, each substituent independently selected from a halogen atom, a C₁-C₂-alkyl group, a C₁-C₂-haloalkyl group, a C₁-C₂-alkoxy group, a C₁-C₃-hydroxyalkyl group and a phenyl group;
or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is a hydrogen atom;
R² is a hydrogen atom;
R³ is selected from a hydrogen atom and a fluorine atom;
R⁴ is a hydrogen atom;
R⁵ is a hydrogen atom;
R⁶ is selected from a phenyl group, a naphthyl group and a heteroaryl group,
   wherein said phenyl, naphthyl or heteroaryl group is each optionally substituted with one or more substituents independently selected from a halogen atom, a hydroxy group, a C₁-C₆-alkyl group, a C₁-C₆-hydroxyalkyl group, a C₁-C₆-haloalkyl group, a C₁-C₆-alkoxy group, a (C₁-C₃-alkyl)-O—(C₁-C₂-alkyl)- group, a (C₁-C₃-alkyl)-O—(C₁-C₂-alkyl)-O— group, a (C₃-C₃-cycloalkyl)-(C₁-C₂-alkyl)-O— group, a (C₃-C₃-halocycloalkyl)-(C₁-C₂-alkyl)-O— group, a (phenyl)-(C₁-C₂-alkyl)-O— group, a C₁-C₆-haloalkoxy group, a C₂-C₆-alkenyl group, a C₃-C₃-cycloalkyl group, a C₃-C₃-cycloalkoxy group, a C₁-C₆-thioalkyl group, a heterocycloalkyl group, a R⁷R⁸N— group, a (C₁-C₂-alkyl)-S(═O)₂— group, a (phenyl)-S(═O)₂— group, a (R⁷R⁸N)—S(═O)₂— group, a (H₃C)—(C═O)— group, a (R⁷R⁸N)—(C═O)— group, a (R⁷R⁸N)—(C═O)—O— group, a R⁹OOC—(C₁-C₃-alkyl)- group, a R⁹OOC— group, a phenyl group, a (phenyl)-O— group and a heteroaryl group,
      wherein said heterocycloalkyl group is connected to the rest of the molecule via a carbon atom of said heterocycloalkyl group,
      wherein said phenyl, heteroaryl or heterocycloalkyl group is each optionally substituted with one or more substituents independently selected from a halogen atom, a C₁-C₃-alkyl group, a phenyl group and a R⁹OOC— group;
R⁷ and R³ are each independently selected from a hydrogen atom, a C₁-C₆-alkyl group, a C₃-C₃-cycloalkyl group and a phenyl group,
   wherein said phenyl group is optionally substituted with one or more substituents independently selected from a halogen atom, a C₁-C₃-alkyl group, a C₁-C₃-haloalkyl group and a C₁-C₃-alkoxy group;
or R⁷ and R⁸ together with the nitrogen atom to which they are attached form a 4- to 7-membered nitrogen-containing heterocycloalkyl group, wherein said heterocycloalkyl group optionally contains one, two or three further heteroatoms independently selected from nitrogen, oxygen and sulfur and is optionally substituted with one or more substituents independently selected from a halogen atom, a C₁-C₃-alkyl group, a C₁-C₃-haloalkyl group, a C₁-C₃-alkoxy group and a phenyl group;
R⁹ is selected from a hydrogen atom, a C₁-C₃-alkyl group and a C₃-C₅-cycloalkyl group;
R^{a} and R^{b} together with the nitrogen atom to which they are attached form a 4-membered nitrogen-containing heterocycloalkyl group which is optionally substituted one, two or three times, each substituent independently selected from a halogen atom, a C₁-C₂-haloalkyl group and a C₁-C₃-hydroxyalkyl group;
or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is a hydrogen atom;
R² is a hydrogen atom;
R³ is selected from a hydrogen atom and a fluorine atom;
R⁴ is a hydrogen atom;
R⁵ is a hydrogen atom;
R⁶ is selected from a phenyl group, a naphthyl group and a heteroaryl group,
   wherein said phenyl, naphthyl or heteroaryl group is each optionally substituted with one or more substituents independently selected from a halogen atom, a hydroxy group, a C₁-C₆-alkyl group, a C₁-C₆-hydroxyalkyl group, a C₁-C₆-haloalkyl group, a C₁-C₆-alkoxy group, a (C₁-C₃-alkyl)-O—(C₁-C₂-alkyl)- group, a (C₁-C₃-alkyl)-O—(C₁-C₂-alkyl)-O— group, a (C₃-C₃-cycloalkyl)-(C₁-C₂-alkyl)-O— group, a (C₃-C₃-halocycloalkyl)-(C₁-C₂-alkyl)-O— group, a (phenyl)-(C₁-C₂-alkyl)-O— group, a C₁-C₆-haloalkoxy group, a C₂-C₆-alkenyl group, a C₃-C₃-cycloalkyl group, a C₃-C₃-cycloalkoxy group, a C₁-C₆-thioalkyl group, a heterocycloalkyl group, a R⁷R⁸N— group, a (C₁-C₂-alkyl)-S(═O)₂— group, a (H₃C)—(C═O)— group, a (R⁷R⁸N)—(C═O)— group, a (R⁷R⁸N)—(C═O)—O— group, a R⁹OOC—(C₁-C₃-alkyl)- group, a R⁹OOC— group, a phenyl group, a (phenyl)-O— group and a heteroaryl group,
      wherein said heterocycloalkyl group is connected to the rest of the molecule via a carbon atom of said heterocycloalkyl group,
      wherein said phenyl, heteroaryl or heterocycloalkyl group is each optionally substituted with one or more substituents independently selected from a halogen atom and a C₁-C₃-alkyl group;
R⁷ and R³ are each independently selected from a hydrogen atom, a C₁-C₆-alkyl group, a C₃-C₃-cycloalkyl group and a phenyl group,
   wherein said phenyl group is optionally substituted with one or more substituents independently selected from a halogen atom, a C₁-C₃-alkyl group, a C₁-C₃-haloalkyl group and a C₁-C₃-alkoxy group;
or R⁷ and R⁸ together with the nitrogen atom to which they are attached form a 4- to 7-membered nitrogen-containing heterocycloalkyl group, wherein said heterocycloalkyl group optionally contains one, two or three further heteroatoms independently selected from nitrogen, oxygen and sulfur and is optionally substituted with one or more substituents independently selected from a halogen atom, a C₁-C₃-alkyl group, a C₁-C₃-haloalkyl group, a C₁-C₃-alkoxy group and a phenyl group;
R⁹ is selected from a hydrogen atom, a C₁-C₃-alkyl group and a C₃-C₆-cycloalkyl group;
R^{a} and R^{b} together with the nitrogen atom to which they are attached form a 4-membered nitrogen-containing heterocycloalkyl group which is optionally substituted one, two or three times, each substituent independently selected from a halogen atom, a C₁-C₂-haloalkyl group and a C₁-C₃-hydroxyalkyl group;
or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is a hydrogen atom;
R² is a hydrogen atom;
R³ is selected from a hydrogen atom and a fluorine atom;
R⁴ is a hydrogen atom;
R⁵ is a hydrogen atom;
R⁶ is selected from a phenyl group, a naphthyl group and a heteroaryl group,
   wherein said phenyl, naphthyl or heteroaryl group is each optionally substituted with one or more substituents independently selected from a halogen atom, a C₁-C₆-alkyl group, a C₁-C₆-haloalkyl group, a C₁-C₆-alkoxy group, a (C₁-C₃-alkyl)-O—(C₁-C₂-alkyl)- group, a (C₃-C₃-cycloalkyl)-(C₁-C₂-alkyl)-O— group, a (C₃-C₃-halocycloalkyl)-(C₁-C₂-alkyl)-O— group, a (phenyl)-(C₁-C₂-alkyl)-O— group, a C₁-C₆-haloalkoxy group, a C₂-C₆-alkenyl group, a C₃-C₃-cycloalkoxy group, a C₁-C₆-thioalkyl group, a heterocycloalkyl group, a R⁷R⁸N— group, a (C₁-C₂-alkyl)-S(═O)₂— group, a (R⁷R⁸N)—(C═O)— group, a (R⁷R⁸N)—(C═O)—O— group, a R⁹OOC—(C₁-C₃-alkyl)- group, a R⁹OOC— group, a phenyl group, a (phenyl)-O— group and a heteroaryl group,
      wherein said heterocycloalkyl group is connected to the rest of the molecule via a carbon atom of said heterocycloalkyl group,
      wherein said phenyl or heteroaryl group is each optionally substituted with one or more substituents independently selected from a halogen atom and a C₁-C₃-alkyl group;
R⁷ and R³ are each independently selected from a hydrogen atom, a C₁-C₆-alkyl group, a C₃-C₃-cycloalkyl group and a phenyl group,
   wherein said phenyl group is optionally substituted with one or more substituents independently selected from a halogen atom, a C₁-C₃-alkyl group, a C₁-C₃-haloalkyl group and a C₁-C₃-alkoxy group;
or R⁷ and R^{a} together with the nitrogen atom to which they are attached form a 4- to 7-membered nitrogen-containing heterocycloalkyl group, wherein said heterocycloalkyl group optionally contains one, two or three further heteroatoms independently selected from nitrogen, oxygen and sulfur and is optionally substituted with one or more substituents independently selected from a halogen atom, a C₁-C₃-alkyl group, a C₁-C₃-haloalkyl group, a C₁-C₃-alkoxy group and a phenyl group;
R⁹ is selected from a hydrogen atom, a C₁-C₃-alkyl group and a C₃-C₅-cycloalkyl group;
R^{a} and R^{b} together with the nitrogen atom to which they are attached form a 4-membered nitrogen-containing heterocycloalkyl group which is optionally substituted one, two or three times, each substituent independently selected from a halogen atom, a C₁-C₂-haloalkyl group and a C₁-C₃-hydroxyalkyl group;
or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is a hydrogen atom;
R² is a hydrogen atom;
R³ is a fluorine atom;
R⁴ is a hydrogen atom;
R⁵ is a hydrogen atom;
R⁶ is selected from a phenyl group, a naphthyl group and a heteroaryl group,
   wherein said phenyl, naphthyl or heteroaryl group is each optionally substituted with one or more substituents independently selected from a halogen atom, a C₁-C₆-alkyl group, a C₁-C₆-haloalkyl group, a C₁-C₆-alkoxy group, a (C₁-C₃-alkyl)-O—(C₁-C₂-alkyl)- group, a (C₃-C₃-cycloalkyl)-(C₁-C₂-alkyl)-O— group, a (C₃-C₃-halocycloalkyl)-(C₁-C₂-alkyl)-O— group, a (phenyl)-(C₁-C₂-alkyl)-O— group, a C₁-C₆-haloalkoxy group, a C₂-C₆-alkenyl group, a C₃-C₃-cycloalkoxy group, a C₁-C₆-thioalkyl group, a heterocycloalkyl group, a R⁷R⁸N— group, a (C₁-C₂-alkyl)-S(═O)₂— group, a (R⁷R⁸N)—(C═O)— group, a (R⁷R⁸N)—(C═O)—O— group, a R⁹OOC—(C₁-C₃-alkyl)- group, a R⁹OOC— group, a phenyl group, a (phenyl)-O— group and a heteroaryl group,
      wherein said heterocycloalkyl group is connected to the rest of the molecule via a carbon atom of said heterocycloalkyl group,
      wherein said phenyl or heteroaryl group is each optionally substituted with one or more substituents independently selected from a halogen atom and a C₁-C₃-alkyl group;
R⁷ and R⁸ are each independently selected from a hydrogen atom, a C₁-C₆-alkyl group, a C₃-C₃-cycloalkyl group and a phenyl group,
   wherein said phenyl group is optionally substituted with one or more substituents independently selected from a halogen atom, a C₁-C₃-alkyl group, a C₁-C₃-haloalkyl group and a C₁-C₃-alkoxy group;
or R⁷ and R^{a} together with the nitrogen atom to which they are attached form a 4- to 7-membered nitrogen-containing heterocycloalkyl group, wherein said heterocycloalkyl group optionally contains one, two or three further heteroatoms independently selected from nitrogen, oxygen and sulfur and is optionally substituted with one or more substituents independently selected from a halogen atom, a C₁-C₃-alkyl group, a C₁-C₃-haloalkyl group, a C₁-C₃-alkoxy group and a phenyl group;
R⁹ is selected from a hydrogen atom, a C₁-C₃-alkyl group and a C₃-C₆-cycloalkyl group;
R^{a} and R^{b} together with the nitrogen atom to which they are attached form a 4-membered nitrogen-containing heterocycloalkyl group which is optionally substituted one, two or three times, each substituent independently selected from a halogen atom, a C₁-C₂-haloalkyl group and a C₁-C₃-hydroxyalkyl group;
or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is a hydrogen atom;
R² is a hydrogen atom;
R³ is selected from a hydrogen atom and a fluorine atom;
R⁴ is a hydrogen atom;
R⁵ is a hydrogen atom;
R⁶ is selected from a phenyl group and a heteroaryl group,
   wherein said phenyl or heteroaryl group is each optionally substituted with one or more substituents independently selected from a halogen atom, a C₁-C₆-alkyl group, a C₁-C₆-haloalkyl group, a C₁-C₆-alkoxy group, a (C₃-C₃-cycloalkyl)-(C₁-C₂-alkyl)-O— group, a (C₃-C₃-halocycloalkyl)-(C₁-C₂-alkyl)-O— group, a (phenyl)-(C₁-C₂-alkyl)-O— group, a C₁-C₆-haloalkoxy group, a C₃-C₃-cycloalkoxy group, a heterocycloalkyl group, a R⁷R⁸N— group, a (phenyl)-O— group and a heteroaryl group,
      wherein said heterocycloalkyl group is connected to the rest of the molecule via a carbon atom of said heterocycloalkyl group,
      wherein said heteroaryl or heterocycloalkyl group is each optionally substituted with one or more substituents independently selected from a C₁-C₃-alkyl group and a phenyl group;
R⁷ and R⁸ are each independently selected from a hydrogen atom, a C₁-C₆-alkyl group, a C₃-C₃-cycloalkyl group and a phenyl group,
   wherein said phenyl group is optionally substituted with one or more substituents independently selected from a halogen atom, a C₁-C₃-alkyl group, a C₁-C₃-haloalkyl group and a C₁-C₃-alkoxy group;
or R⁷ and R^{a} together with the nitrogen atom to which they are attached form a 4- to 7-membered nitrogen-containing heterocycloalkyl group, wherein said heterocycloalkyl group optionally contains one, two or three further heteroatoms independently selected from nitrogen, oxygen and sulfur and is optionally substituted with one or more substituents independently selected from a halogen atom, a C₁-C₃-alkyl group, a C₁-C₃-haloalkyl group, a C₁-C₃-alkoxy group and a phenyl group;
R⁹ is selected from a hydrogen atom, a C₁-C₃-alkyl group and a C₃-C₆-cycloalkyl group;
R^{a} and R^{b} together with the nitrogen atom to which they are attached form a 4-membered nitrogen-containing heterocycloalkyl group which is optionally substituted one, two or three times, each substituent independently selected from a halogen atom, a C₁-C₂-haloalkyl group and a C₁-C₃-hydroxyalkyl group;
or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is a hydrogen atom;
R² is a hydrogen atom;
R³ is selected from a hydrogen atom and a fluorine atom;
R⁴ is a hydrogen atom;
R⁵ is a hydrogen atom;
R⁶ is selected from a phenyl group and a heteroaryl group,
   wherein said phenyl or heteroaryl group is each optionally substituted with one or more substituents independently selected from a halogen atom, a C₁-C₆-alkyl group, a C₁-C₆-alkoxy group, a (phenyl)-(C₁-C₂-alkyl)-O— group, a C₁-C₆-haloalkoxy group, a C₂-C₆-alkenyl group, a C₃-C₃-cycloalkoxy group, a heterocycloalkyl group, a phenyl group,
      wherein said heterocycloalkyl group is connected to the rest of the molecule via a carbon atom of said heterocycloalkyl group,
      wherein said phenyl, or heterocycloalkyl group is each optionally substituted with one or more substituents independently selected from a halogen atom, a C₁-C₃-alkyl group and a R⁹OOC— group;
R⁷ and R³ are each independently selected from a hydrogen atom, a C₁-C₆-alkyl group, a C₃-C₃-cycloalkyl group and a phenyl group,
   wherein said phenyl group is optionally substituted with one or more substituents independently selected from a halogen atom, a C₁-C₃-alkyl group, a C₁-C₃-haloalkyl group and a C₁-C₃-alkoxy group;
or R⁷ and R^{a} together with the nitrogen atom to which they are attached form a 4- to 7-membered nitrogen-containing heterocycloalkyl group, wherein said heterocycloalkyl group optionally contains one, two or three further heteroatoms independently selected from nitrogen, oxygen and sulfur and is optionally substituted with one or more substituents independently selected from a halogen atom, a C₁-C₃-alkyl group, a C₁-C₃-haloalkyl group, a C₁-C₃-alkoxy group and a phenyl group;
R⁹ is selected from a hydrogen atom, a C₁-C₃-alkyl group and a C₃-C₅-cycloalkyl group;
R^{a} and R^{b} together with the nitrogen atom to which they are attached form a 4-membered nitrogen-containing heterocycloalkyl group which is optionally substituted one, two or three times, each substituent independently selected from a halogen atom, a C₁-C₂-haloalkyl group and a C₁-C₃-hydroxyalkyl group;
or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is a hydrogen atom;
R² is a hydrogen atom;
R³ is selected from a hydrogen atom and a fluorine atom;
R⁴ is a hydrogen atom;
R⁵ is a hydrogen atom;
R⁶ is selected from a phenyl group, a naphthyl group and a heteroaryl group,
   wherein said phenyl, naphthyl or heteroaryl group is each optionally substituted with one or more substituents independently selected from a halogen atom, a C₁-C₆-alkyl group, a C₁-C₆-haloalkyl group, a C₁-C₆-alkoxy group, a (C₁-C₃-alkyl)-O—(C₁-C₂-alkyl)- group, a (C₁-C₃-alkyl)-O—(C₁-C₂-alkyl)-O— group, a C₂-C₆-alkenyl group, a C₁-C₆-thioalkyl group, a heterocycloalkyl group, a R⁷R⁸N— group, a (C₁-C₂-alkyl)-S(═O)₂— group, a (R⁷R⁸N)—(C═O)— group, a (R⁷R⁸N)—(C═O)—O— group, a R⁹OOC—(C₁-C₃-alkyl)- group, a R⁹OOC— group, and a heteroaryl group,
      wherein said heterocycloalkyl group is connected to the rest of the molecule via a carbon atom of said heterocycloalkyl group,
      wherein said heteroaryl group is each optionally substituted with one or more substituents independently selected from a C₁-C₆-alkyl group;
R⁷ and R⁸ are each independently selected from a hydrogen atom, a C₁-C₆-alkyl group, a C₃-C₃-cycloalkyl group and a phenyl group,
   wherein said phenyl group is optionally substituted with one or more substituents independently selected from a halogen atom, a C₁-C₃-alkyl group, a C₁-C₃-haloalkyl group and a C₁-C₃-alkoxy group;
or R⁷ and R^{a} together with the nitrogen atom to which they are attached form a 4- to 7-membered nitrogen-containing heterocycloalkyl group, wherein said heterocycloalkyl group optionally contains one, two or three further heteroatoms independently selected from nitrogen, oxygen and sulfur and is optionally substituted with one or more substituents independently selected from a halogen atom, a C₁-C₃-alkyl group, a C₁-C₃-haloalkyl group, a C₁-C₃-alkoxy group and a phenyl group;
R⁹ is selected from a hydrogen atom, a C₁-C₃-alkyl group and a C₃-C₆-cycloalkyl group;
R^{a} and R^{b} together with the nitrogen atom to which they are attached form a 4-membered nitrogen-containing heterocycloalkyl group which is optionally substituted one, two or three times, each substituent independently selected from a halogen atom, a C₁-C₂-haloalkyl group and a C₁-C₃-hydroxyalkyl group;
or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is a hydrogen atom;
R² is a hydrogen atom;
R³ is selected from a hydrogen atom and a fluorine atom;
R⁴ is a hydrogen atom;
R⁵ is a hydrogen atom;
R⁶ is selected from a phenyl group, a naphthyl group and a heteroaryl group,
   wherein said phenyl, naphthyl or heteroaryl group is each optionally substituted with one or more substituents independently selected from a halogen atom, a hydroxy group, a C₁-C₆-alkyl group, a C₁-C₆-hydroxyalkyl group, a C₁-C₆-haloalkyl group, a C₁-C₆-alkoxy group, a (C₁-C₃-alkyl)-O—(C₁-C₂-alkyl)- group, a (C₁-C₃-alkyl)-O—(C₁-C₂-alkyl)-O— group, a (C₃-C₃-cycloalkyl)-(C₁-C₂-alkyl)-O— group, a (C₃-C₃-halocycloalkyl)-(C₁-C₂-alkyl)-O— group, a (phenyl)-(C₁-C₂-alkyl)-O— group, a C₁-C₆-haloalkoxy group, a C₂-C₆-alkenyl group, a C₃-C₃-cycloalkyl group, a C₃-C₃-cycloalkoxy group, a C₁-C₆-thioalkyl group, a heterocycloalkyl group, a R⁷R⁸N— group, a (C₁-C₂-alkyl)-S(═O)₂— group, a (phenyl)-S(═O)₂— group, a (R⁷R⁸N)—S(═O)₂— group, a (H₃C)—(C═O)— group, a (R⁷R⁸N)—(C═O)— group, a (R⁷R⁸N)—(C═O)—O— group, a R⁹OOC—(C₁-C₃-alkyl)- group, a R⁹OOC— group, a phenyl group, a (phenyl)-O— group and a heteroaryl group,
      wherein said heterocycloalkyl group is connected to the rest of the molecule via a carbon atom of said heterocycloalkyl group,
      wherein said phenyl, heteroaryl or heterocycloalkyl group is each optionally substituted with one or more substituents independently selected from a halogen atom, a C₁-C₃-alkyl group, a phenyl group and a R⁹OOC— group;
R⁷ and R⁸ are each independently selected from a hydrogen atom, a C₁-C₆-alkyl group, a C₃-C₃-cycloalkyl group and a phenyl group,
   wherein said phenyl group is optionally substituted with one or more substituents independently selected from a halogen atom, a C₁-C₃-alkyl group, a C₁-C₃-haloalkyl group and a C₁-C₃-alkoxy group;
or R⁷ and R^{a} together with the nitrogen atom to which they are attached form a 4- to 7-membered nitrogen-containing heterocycloalkyl group, wherein said heterocycloalkyl group optionally contains one, two or three further heteroatoms independently selected from nitrogen, oxygen and sulfur and is optionally substituted with one or more substituents independently selected from a halogen atom, a C₁-C₃-alkyl group, a C₁-C₃-haloalkyl group, a C₁-C₃-alkoxy group and a phenyl group;
R⁹ is selected from a hydrogen atom, a C₁-C₃-alkyl group and a C₃-C₅-cycloalkyl group;
R^{a} and R^{b} together with the nitrogen atom to which they are attached form a 4-membered nitrogen-containing heterocycloalkyl group which is optionally substituted one, two or three times with a halogen atom;
or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is a hydrogen atom;
R² is a hydrogen atom;
R³ is selected from a hydrogen atom and a fluorine atom;
R⁴ is a hydrogen atom;
R⁵ is a hydrogen atom;
R⁶ is selected from a phenyl group, a naphthyl group and a heteroaryl group,
   wherein said phenyl, naphthyl or heteroaryl group is each optionally substituted with one or more substituents independently selected from a halogen atom, a hydroxy group, a C₁-C₆-alkyl group, a C₁-C₆-hydroxyalkyl group, a C₁-C₆-haloalkyl group, a C₁-C₆-alkoxy group, a (C₁-C₃-alkyl)-O—(C₁-C₂-alkyl)- group, a (C₁-C₃-alkyl)-O—(C₁-C₂-alkyl)-O— group, a (C₃-C₃-cycloalkyl)-(C₁-C₂-alkyl)-O— group, a (C₃-C₃-halocycloalkyl)-(C₁-C₂-alkyl)-O— group, a (phenyl)-(C₁-C₂-alkyl)-O— group, a C₁-C₆-haloalkoxy group, a C₂-C₆-alkenyl group, a C₃-C₃-cycloalkyl group, a C₃-C₃-cycloalkoxy group, a C₁-C₆-thioalkyl group, a heterocycloalkyl group, a R⁷R⁸N— group, a (C₁-C₂-alkyl)-S(═O)₂— group, a (H₃C)—(C═O)— group, a (R⁷R⁸N)—(C═O)— group, a (R⁷R⁸N)—(C═O)—O— group, a R⁹OOC—(C₁-C₃-alkyl)- group, a R⁹OOC— group, a phenyl group, a (phenyl)-O— group and a heteroaryl group,
      wherein said heterocycloalkyl group is connected to the rest of the molecule via a carbon atom of said heterocycloalkyl group,
      wherein said phenyl, heteroaryl or heterocycloalkyl group is each optionally substituted with one or more substituents independently selected from a halogen atom and a C₁-C₃-alkyl group;
R⁷ and R⁸ are each independently selected from a hydrogen atom, a C₁-C₆-alkyl group, a C₃-C₃-cycloalkyl group and a phenyl group,
   wherein said phenyl group is optionally substituted with one or more substituents independently selected from a halogen atom, a C₁-C₃-alkyl group, a C₁-C₃-haloalkyl group and a C₁-C₃-alkoxy group;
or R⁷ and R^{a} together with the nitrogen atom to which they are attached form a 4- to 7-membered nitrogen-containing heterocycloalkyl group, wherein said heterocycloalkyl group optionally contains one, two or three further heteroatoms independently selected from nitrogen, oxygen and sulfur and is optionally substituted with one or more substituents independently selected from a halogen atom, a C₁-C₃-alkyl group, a C₁-C₃-haloalkyl group, a C₁-C₃-alkoxy group and a phenyl group;
R⁹ is selected from a hydrogen atom, a C₁-C₃-alkyl group and a C₃-C₆-cycloalkyl group;
R^{a} and R^{b} together with the nitrogen atom to which they are attached form a 4-membered nitrogen-containing heterocycloalkyl group which is optionally substituted one, two or three times with a halogen atom;
or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is a hydrogen atom;
R² is a hydrogen atom;
R³ is selected from a hydrogen atom and a fluorine atom;
R⁴ is a hydrogen atom;
R⁵ is a hydrogen atom;
R⁶ is selected from a phenyl group, a naphthyl group and a heteroaryl group,
   wherein said phenyl, naphthyl or heteroaryl group is each optionally substituted with one or more substituents independently selected from a halogen atom, a C₁-C₆-alkyl group, a C₁-C₆-haloalkyl group, a C₁-C₆-alkoxy group, a (C₁-C₃-alkyl)-O—(C₁-C₂-alkyl)- group, a (C₃-C₃-cycloalkyl)-(C₁-C₂-alkyl)-O— group, a (C₃-C₃-halocycloalkyl)-(C₁-C₂-alkyl)-O— group, a (phenyl)-(C₁-C₂-alkyl)-O— group, a C₁-C₆-haloalkoxy group, a C₂-C₆-alkenyl group, a C₃-C₃-cycloalkoxy group, a C₁-C₆-thioalkyl group, a heterocycloalkyl group, a R⁷R⁸N— group, a (C₁-C₂-alkyl)-S(═O)₂— group, a (R⁷R⁸N)—(C═O)— group, a (R⁷R⁸N)—(C═O)—O— group, a R⁹OOC—(C₁-C₃-alkyl)- group, a R⁹OOC— group, a phenyl group, a (phenyl)-O— group and a heteroaryl group,
      wherein said heterocycloalkyl group is connected to the rest of the molecule via a carbon atom of said heterocycloalkyl group,
      wherein said phenyl or heteroaryl group is each optionally substituted with one or more substituents independently selected from a halogen atom and a C₁-C₃-alkyl group;
R⁷ and R⁸ are each independently selected from a hydrogen atom, a C₁-C₆-alkyl group, a C₃-C₃-cycloalkyl group and a phenyl group,
   wherein said phenyl group is optionally substituted with one or more substituents independently selected from a halogen atom, a C₁-C₃-alkyl group, a C₁-C₃-haloalkyl group and a C₁-C₃-alkoxy group;
or R⁷ and R^{a} together with the nitrogen atom to which they are attached form a 4- to 7-membered nitrogen-containing heterocycloalkyl group, wherein said heterocycloalkyl group optionally contains one, two or three further heteroatoms independently selected from nitrogen, oxygen and sulfur and is optionally substituted with one or more substituents independently selected from a halogen atom, a C₁-C₃-alkyl group, a C₁-C₃-haloalkyl group, a C₁-C₃-alkoxy group and a phenyl group;
R⁹ is selected from a hydrogen atom, a C₁-C₃-alkyl group and a C₃-C₆-cycloalkyl group;
R^{a} and R^{b} together with the nitrogen atom to which they are attached form a 4-membered nitrogen-containing heterocycloalkyl group which is optionally substituted one, two or three times with a halogen atom;
or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is a hydrogen atom;
R² is a hydrogen atom;
R³ is selected from a hydrogen atom and a fluorine atom;
R⁴ is a hydrogen atom;
R⁵ is a hydrogen atom;
R⁶ is selected from a phenyl group, a naphthyl group and a heteroaryl group,
   wherein said phenyl, naphthyl or heteroaryl group is each optionally substituted with one or more substituents independently selected from a halogen atom, a C₁-C₆-alkyl group, a C₁-C₆-haloalkyl group, a C₁-C₆-alkoxy group, a (C₁-C₃-alkyl)-O—(C₁-C₂-alkyl)- group, a (C₃-C₃-cycloalkyl)-(C₁-C₂-alkyl)-O— group, a (C₃-C₃-halocycloalkyl)-(C₁-C₂-alkyl)-O— group, a (phenyl)-(C₁-C₂-alkyl)-O— group, a C₁-C₆-haloalkoxy group, a C₂-C₆-alkenyl group, a C₃-C₃-cycloalkoxy group, a C₁-C₆-thioalkyl group, a heterocycloalkyl group, a R⁷R⁸N— group, a (C₁-C₂-alkyl)-S(═O)₂— group, a (R⁷R⁸N)—(C═O)— group, a (R⁷R⁸N)—(C═O)—O— group, a R⁹OOC—(C₁-C₃-alkyl)- group, a R⁹OOC— group, a phenyl group, a (phenyl)-O— group and a heteroaryl group,
      wherein said heterocycloalkyl group is connected to the rest of the molecule via a carbon atom of said heterocycloalkyl group,
      wherein said phenyl or heteroaryl group is each optionally substituted with one or more substituents independently selected from a halogen atom and a C₁-C₃-alkyl group;
R⁷ and R³ are each independently selected from a hydrogen atom, a C₁-C₆-alkyl group, a C₃-C₃-cycloalkyl group and a phenyl group,
   wherein said phenyl group is optionally substituted with one or more substituents independently selected from a halogen atom, a C₁-C₃-alkyl group, a C₁-C₃-haloalkyl group and a C₁-C₃-alkoxy group;
or R⁷ and R⁸ together with the nitrogen atom to which they are attached form a 4- to 7-membered nitrogen-containing heterocycloalkyl group, wherein said heterocycloalkyl group optionally contains one, two or three further heteroatoms independently selected from nitrogen, oxygen and sulfur and is optionally substituted with one or more substituents independently selected from a halogen atom, a C₁-C₃-alkyl group, a C₁-C₃-haloalkyl group, a C₁-C₃-alkoxy group and a phenyl group;
R⁹ is selected from a hydrogen atom, a C₁-C₃-alkyl group and a C₃-C₅-cycloalkyl group;
R^{a} and R^{b} together with the nitrogen atom to which they are attached form an unsubstituted 4- membered nitrogen-containing heterocycloalkyl group;
or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is a hydrogen atom;
R² is a hydrogen atom;
R³ is a fluorine atom;
R⁴ is a hydrogen atom;
R⁵ is a hydrogen atom;
R⁶ is selected from a phenyl group, a naphthyl group and a heteroaryl group,
   wherein said phenyl, naphthyl or heteroaryl group is each optionally substituted with one or more substituents independently selected from a halogen atom, a C₁-C₆-alkyl group, a C₁-C₆-haloalkyl group, a C₁-C₆-alkoxy group, a (C₁-C₃-alkyl)-O—(C₁-C₂-alkyl)- group, a (C₃-C₃-cycloalkyl)-(C₁-C₂-alkyl)-O— group, a (C₃-C₃-halocycloalkyl)-(C₁-C₂-alkyl)-O— group, a (phenyl)-(C₁-C₂-alkyl)-O— group, a C₁-C₆-haloalkoxy group, a C₂-C₆-alkenyl group, a C₃-C₃-cycloalkoxy group, a C₁-C₆-thioalkyl group, a heterocycloalkyl group, a R⁷R⁸N— group, a (C₁-C₂-alkyl)-S(═O)₂— group, a (R⁷R⁸N)—(C═O)— group, a (R⁷R⁸N)—(C═O)—O— group, a R⁹OOC—(C₁-C₃-alkyl)- group, a R⁹OOC— group, a phenyl group, a (phenyl)-O— group and a heteroaryl group,
      wherein said heterocycloalkyl group is connected to the rest of the molecule via a carbon atom of said heterocycloalkyl group,
      wherein said phenyl or heteroaryl group is each optionally substituted with one or more substituents independently selected from a halogen atom and a C₁-C₃-alkyl group;
R⁷ and R³ are each independently selected from a hydrogen atom, a C₁-C₆-alkyl group, a C₃-C₃-cycloalkyl group and a phenyl group,
   wherein said phenyl group is optionally substituted with one or more substituents independently selected from a halogen atom, a C₁-C₃-alkyl group, a C₁-C₃-haloalkyl group and a C₁-C₃-alkoxy group;
or R⁷ and R^{a} together with the nitrogen atom to which they are attached form a 4- to 7-membered nitrogen-containing heterocycloalkyl group, wherein said heterocycloalkyl group optionally contains one, two or three further heteroatoms independently selected from nitrogen, oxygen and sulfur and is optionally substituted with one or more substituents independently selected from a halogen atom, a C₁-C₃-alkyl group, a C₁-C₃-haloalkyl group, a C₁-C₃-alkoxy group and a phenyl group;
R⁹ is selected from a hydrogen atom, a C₁-C₃-alkyl group and a C₃-C₆-cycloalkyl group;
R^{a} and R^{b} together with the nitrogen atom to which they are attached form a 4-membered nitrogen-containing heterocycloalkyl group which is optionally substituted one, two or three times with a halogen atom;
or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is a hydrogen atom;
R² is a hydrogen atom;
R³ is a fluorine atom;
R⁴ is a hydrogen atom;
R⁵ is a hydrogen atom;
R⁶ is selected from a phenyl group, a naphthyl group and a heteroaryl group,
   wherein said phenyl, naphthyl or heteroaryl group is each optionally substituted with one or more substituents independently selected from a halogen atom, a C₁-C₆-alkyl group, a C₁-C₆-haloalkyl group, a C₁-C₆-alkoxy group, a (C₁-C₃-alkyl)-O—(C₁-C₂-alkyl)- group, a (C₃-C₃-cycloalkyl)-(C₁-C₂-alkyl)-O— group, a (C₃-C₃-halocycloalkyl)-(C₁-C₂-alkyl)-O— group, a (phenyl)-(C₁-C₂-alkyl)-O— group, a C₁-C₆-haloalkoxy group, a C₂-C₆-alkenyl group, a C₃-C₃-cycloalkoxy group, a C₁-C₆-thioalkyl group, a heterocycloalkyl group, a R⁷R⁸N— group, a (C₁-C₂-alkyl)-S(═O)₂— group, a (R⁷R⁸N)—(C═O)— group, a (R⁷R⁸N)—(C═O)—O— group, a R⁹OOC—(C₁-C₃-alkyl)- group, a R⁹OOC— group, a phenyl group, a (phenyl)-O— group and a heteroaryl group,
      wherein said heterocycloalkyl group is connected to the rest of the molecule via a carbon atom of said heterocycloalkyl group,
      wherein said phenyl or heteroaryl group is each optionally substituted with one or more substituents independently selected from a halogen atom and a C₁-C₃-alkyl group;
R⁷ and R³ are each independently selected from a hydrogen atom, a C₁-C₆-alkyl group, a C₃-C₃-cycloalkyl group and a phenyl group,
   wherein said phenyl group is optionally substituted with one or more substituents independently selected from a halogen atom, a C₁-C₃-alkyl group, a C₁-C₃-haloalkyl group and a C₁-C₃-alkoxy group;
or R⁷ and R^{a} together with the nitrogen atom to which they are attached form a 4- to 7-membered nitrogen-containing heterocycloalkyl group, wherein said heterocycloalkyl group optionally contains one, two or three further heteroatoms independently selected from nitrogen, oxygen and sulfur and is optionally substituted with one or more substituents independently selected from a halogen atom, a C₁-C₃-alkyl group, a C₁-C₃-haloalkyl group, a C₁-C₃-alkoxy group and a phenyl group;
R⁹ is selected from a hydrogen atom, a C₁-C₃-alkyl group and a C₃-C₅-cycloalkyl group;
R^{a} and R^{b} together with the nitrogen atom to which they are attached form a 4-membered nitrogen-containing heterocycloalkyl group which is optionally substituted one time with a fluorine atom;
or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is a hydrogen atom;
R² is a hydrogen atom;
R³ is selected from a hydrogen atom and a fluorine atom;
R⁴ is a hydrogen atom;
R⁵ is a hydrogen atom;
R⁶ is selected from a phenyl group and a heteroaryl group,
   wherein said phenyl or heteroaryl group is each optionally substituted with one or more substituents independently selected from a halogen atom, a C₁-C₆-alkyl group, a C₁-C₆-haloalkyl group, a C₁-C₆-alkoxy group, a (C₃-C₃-cycloalkyl)-(C₁-C₂-alkyl)-O— group, a (C₃-C₃-halocycloalkyl)-(C₁-C₂-alkyl)-O— group, a (phenyl)-(C₁-C₂-alkyl)-O— group, a C₁-C₆-haloalkoxy group, a C₃-C₃-cycloalkoxy group, a heterocycloalkyl group, a R⁷R⁸N— group, a (phenyl)-O— group and a heteroaryl group,
      wherein said heterocycloalkyl group is connected to the rest of the molecule via a carbon atom of said heterocycloalkyl group,
      wherein said heteroaryl or heterocycloalkyl group is each optionally substituted with one or more substituents independently selected from a C₁-C₃-alkyl group and a phenyl group;
R⁷ and R⁸ are each independently selected from a hydrogen atom, a C₁-C₆-alkyl group, a C₃-C₃-cycloalkyl group and a phenyl group,
   wherein said phenyl group is optionally substituted with one or more substituents independently selected from a halogen atom, a C₁-C₃-alkyl group, a C₁-C₃-haloalkyl group and a C₁-C₃-alkoxy group;
or R⁷ and R⁸ together with the nitrogen atom to which they are attached form a 4- to 7-membered nitrogen-containing heterocycloalkyl group, wherein said heterocycloalkyl group optionally contains one, two or three further heteroatoms independently selected from nitrogen, oxygen and sulfur and is optionally substituted with one or more substituents independently selected from a halogen atom, a C₁-C₃-alkyl group, a C₁-C₃-haloalkyl group, a C₁-C₃-alkoxy group and a phenyl group;
R⁹ is selected from a hydrogen atom, a C₁-C₃-alkyl group and a C₃-C₆-cycloalkyl group;
R^{a} and R^{b} together with the nitrogen atom to which they are attached form a 4-membered nitrogen-containing heterocycloalkyl group which is optionally substituted one, two or three times with a halogen atom;
or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is a hydrogen atom;
R² is a hydrogen atom;
R³ is selected from a hydrogen atom and a fluorine atom;
R⁴ is a hydrogen atom;
R⁵ is a hydrogen atom;
R⁶ is selected from a phenyl group and a heteroaryl group,
   wherein said phenyl or heteroaryl group is each optionally substituted with one or more substituents independently selected from a halogen atom, a C₁-C₆-alkyl group, a C₁-C₆-haloalkyl group, a C₁-C₆-alkoxy group, a (C₃-C₃-cycloalkyl)-(C₁-C₂-alkyl)-O— group, a (C₃-C₃-halocycloalkyl)-(C₁-C₂-alkyl)-O— group, a (phenyl)-(C₁-C₂-alkyl)-O— group, a C₁-C₆-haloalkoxy group, a C₃-C₃-cycloalkoxy group, a heterocycloalkyl group, a R⁷R⁸N— group, a (phenyl)-O— group and a heteroaryl group,
      wherein said heterocycloalkyl group is connected to the rest of the molecule via a carbon atom of said heterocycloalkyl group,
      wherein said heteroaryl or heterocycloalkyl group is each optionally substituted with one or more substituents independently selected from a C₁-C₃-alkyl group and a phenyl group;
R⁷ and R³ are each independently selected from a hydrogen atom, a C₁-C₆-alkyl group, a C₃-C₃-cycloalkyl group and a phenyl group,
   wherein said phenyl group is optionally substituted with one or more substituents independently selected from a halogen atom, a C₁-C₃-alkyl group, a C₁-C₃-haloalkyl group and a C₁-C₃-alkoxy group;
or R⁷ and R^{a} together with the nitrogen atom to which they are attached form a 4- to 7-membered nitrogen-containing heterocycloalkyl group, wherein said heterocycloalkyl group optionally contains one, two or three further heteroatoms independently selected from nitrogen, oxygen and sulfur and is optionally substituted with one or more substituents independently selected from a halogen atom, a C₁-C₃-alkyl group, a C₁-C₃-haloalkyl group, a C₁-C₃-alkoxy group and a phenyl group;
R⁹ is selected from a hydrogen atom, a C₁-C₃-alkyl group and a C₃-C₆-cycloalkyl group;
R^{a} and R^{b} together with the nitrogen atom to which they are attached form an unsubstituted 4- membered nitrogen-containing heterocycloalkyl group;
or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is a hydrogen atom;
R² is a hydrogen atom;
R³ is a fluorine atom;
R⁴ is a hydrogen atom;
R⁵ is a hydrogen atom;
R⁶ is selected from a phenyl group and a heteroaryl group,
   wherein said phenyl or heteroaryl group is each optionally substituted with one or more substituents independently selected from a halogen atom, a C₁-C₆-alkyl group, a C₁-C₆-haloalkyl group, a C₁-C₆-alkoxy group, a (C₃-C₃-cycloalkyl)-(C₁-C₂-alkyl)-O— group, a (C₃-C₃-halocycloalkyl)-(C₁-C₂-alkyl)-O— group, a (phenyl)-(C₁-C₂-alkyl)-O— group, a C₁-C₆-haloalkoxy group, a C₃-C₃-cycloalkoxy group, a heterocycloalkyl group, a R⁷R⁸N— group, a (phenyl)-O— group and a heteroaryl group,
      wherein said heterocycloalkyl group is connected to the rest of the molecule via a carbon atom of said heterocycloalkyl group,
      wherein said heteroaryl or heterocycloalkyl group is each optionally substituted with one or more substituents independently selected from a C₁-C₃-alkyl group and a phenyl group;
R⁷ and R⁸ are each independently selected from a hydrogen atom, a C₁-C₆-alkyl group, a C₃-C₃-cycloalkyl group and a phenyl group,
   wherein said phenyl group is optionally substituted with one or more substituents independently selected from a halogen atom, a C₁-C₃-alkyl group, a C₁-C₃-haloalkyl group and a C₁-C₃-alkoxy group;
or R⁷ and R^{a} together with the nitrogen atom to which they are attached form a 4- to 7-membered nitrogen-containing heterocycloalkyl group, wherein said heterocycloalkyl group optionally contains one, two or three further heteroatoms independently selected from nitrogen, oxygen and sulfur and is optionally substituted with one or more substituents independently selected from a halogen atom, a C₁-C₃-alkyl group, a C₁-C₃-haloalkyl group, a C₁-C₃-alkoxy group and a phenyl group;
R⁹ is selected from a hydrogen atom, a C₁-C₃-alkyl group and a C₃-C₆-cycloalkyl group;
R^{a} and R^{b} together with the nitrogen atom to which they are attached form a 4-membered nitrogen-containing heterocycloalkyl group which is optionally substituted one, two or three times with a halogen atom;
or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is a hydrogen atom;
R² is a hydrogen atom;
R³ is a fluorine atom;
R⁴ is a hydrogen atom;
R⁵ is a hydrogen atom;
R⁶ is selected from a phenyl group and a heteroaryl group,
   wherein said phenyl or heteroaryl group is each optionally substituted with one or more substituents independently selected from a halogen atom, a C₁-C₆-alkyl group, a C₁-C₆-haloalkyl group, a C₁-C₆-alkoxy group, a (C₃-C₃-cycloalkyl)-(C₁-C₂-alkyl)-O— group, a (C₃-C₃-halocycloalkyl)-(C₁-C₂-alkyl)-O— group, a (phenyl)-(C₁-C₂-alkyl)-O— group, a C₁-C₆-haloalkoxy group, a C₃-C₃-cycloalkoxy group, a heterocycloalkyl group, a R⁷R⁸N— group, a (phenyl)-O— group and a heteroaryl group,
      wherein said heterocycloalkyl group is connected to the rest of the molecule via a carbon atom of said heterocycloalkyl group,
      wherein said heteroaryl or heterocycloalkyl group is each optionally substituted with one or more substituents independently selected from a C₁-C₃-alkyl group and a phenyl group;
R⁷ and R³ are each independently selected from a hydrogen atom, a C₁-C₆-alkyl group, a C₃-C₃-cycloalkyl group and a phenyl group,
   wherein said phenyl group is optionally substituted with one or more substituents independently selected from a halogen atom, a C₁-C₃-alkyl group, a C₁-C₃-haloalkyl group and a C₁-C₃-alkoxy group;
or R⁷ and R^{a} together with the nitrogen atom to which they are attached form a 4- to 7-membered nitrogen-containing heterocycloalkyl group, wherein said heterocycloalkyl group optionally contains one, two or three further heteroatoms independently selected from nitrogen, oxygen and sulfur and is optionally substituted with one or more substituents independently selected from a halogen atom, a C₁-C₃-alkyl group, a C₁-C₃-haloalkyl group, a C₁-C₃-alkoxy group and a phenyl group;
R⁹ is selected from a hydrogen atom, a C₁-C₃-alkyl group and a C₃-C₅-cycloalkyl group;
R^{a} and R^{b} together with the nitrogen atom to which they are attached form a 4-membered nitrogen-containing heterocycloalkyl group which is optionally substituted one time with a fluorine atom;
or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is a hydrogen atom;
R² is a hydrogen atom;
R³ is selected from a hydrogen atom and a fluorine atom;
R⁴ is a hydrogen atom;
R⁵ is a hydrogen atom;
R⁶ is selected from a phenyl group and a heteroaryl group,
   wherein said phenyl or heteroaryl group is each optionally substituted with one or more substituents independently selected from a halogen atom, a C₁-C₆-alkyl group, a C₁-C₆-alkoxy group, a (phenyl)-(C₁-C₂-alkyl)-O— group, a C₁-C₆-haloalkoxy group, a C₂-C₆-alkenyl group, a C₃-C₃-cycloalkoxy group, a heterocycloalkyl group, a phenyl group,
      wherein said heterocycloalkyl group is connected to the rest of the molecule via a carbon atom of said heterocycloalkyl group,
      wherein said phenyl, or heterocycloalkyl group is each optionally substituted with one or more substituents independently selected from a halogen atom, a C₁-C₃-alkyl group and a R⁹OOC— group;
R⁷ and R⁸ are each independently selected from a hydrogen atom, a C₁-C₆-alkyl group, a C₃-C₃-cycloalkyl group and a phenyl group,
   wherein said phenyl group is optionally substituted with one or more substituents independently selected from a halogen atom, a C₁-C₃-alkyl group, a C₁-C₃-haloalkyl group and a C₁-C₃-alkoxy group;
or R⁷ and R⁸ together with the nitrogen atom to which they are attached form a 4- to 7-membered nitrogen-containing heterocycloalkyl group, wherein said heterocycloalkyl group optionally contains one, two or three further heteroatoms independently selected from nitrogen, oxygen and sulfur and is optionally substituted with one or more substituents independently selected from a halogen atom, a C₁-C₃-alkyl group, a C₁-C₃-haloalkyl group, a C₁-C₃-alkoxy group and a phenyl group;
R⁹ is selected from a hydrogen atom, a C₁-C₃-alkyl group and a C₃-C₆-cycloalkyl group;
R^{a} and R^{b} together with the nitrogen atom to which they are attached form a 4-membered nitrogen-containing heterocycloalkyl group which is optionally substituted one, two or three times with a halogen atom;
or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is a hydrogen atom;
R² is a hydrogen atom;
R³ is selected from a hydrogen atom and a fluorine atom;
R⁴ is a hydrogen atom;
R⁵ is a hydrogen atom;
R⁶ is selected from a phenyl group, a naphthyl group and a heteroaryl group,
   wherein said phenyl, naphthyl or heteroaryl group is each optionally substituted with one or more substituents independently selected from a halogen atom, a C₁-C₆-alkyl group, a C₁-C₆-haloalkyl group, a C₁-C₆-alkoxy group, a (C₁-C₃-alkyl)-O—(C₁-C₂-alkyl)- group, a (C₁-C₃-alkyl)-O—(C₁-C₂-alkyl)-O— group, a C₂-C₆-alkenyl group, a C₁-C₆-thioalkyl group, a heterocycloalkyl group, a R⁷R⁸N— group, a (C₁-C₂-alkyl)-S(═O)₂— group, a (R⁷R⁸N)—(C═O)— group, a (R⁷R⁸N)—(C═O)—O— group, a R⁹OOC—(C₁-C₃-alkyl)- group, a R⁹OOC— group, and a heteroaryl group,
      wherein said heterocycloalkyl group is connected to the rest of the molecule via a carbon atom of said heterocycloalkyl group,
      wherein said heteroaryl group is each optionally substituted with one or more substituents independently selected from a C₁-C₆-alkyl group;
R⁷ and R³ are each independently selected from a hydrogen atom, a C₁-C₆-alkyl group, a C₃-C₃-cycloalkyl group and a phenyl group,
   wherein said phenyl group is optionally substituted with one or more substituents independently selected from a halogen atom, a C₁-C₃-alkyl group, a C₁-C₃-haloalkyl group and a C₁-C₃-alkoxy group;
or R⁷ and R⁸ together with the nitrogen atom to which they are attached form a 4- to 7-membered nitrogen-containing heterocycloalkyl group, wherein said heterocycloalkyl group optionally contains one, two or three further heteroatoms independently selected from nitrogen, oxygen and sulfur and is optionally substituted with one or more substituents independently selected from a halogen atom, a C₁-C₃-alkyl group, a C₁-C₃-haloalkyl group, a C₁-C₃-alkoxy group and a phenyl group;
R⁹ is selected from a hydrogen atom, a C₁-C₃-alkyl group and a C₃-C₆-cycloalkyl group;
R^{a} and R^{b} together with the nitrogen atom to which they are attached form a 4-membered nitrogen-containing heterocycloalkyl group which is optionally substituted one, two or three times with a halogen atom;
or a tautomer, or an N-oxide, or a salt thereof, or a salt of a tautomer, or a salt of an N-oxide, or a mixture of same.

In accordance with further embodiments, the present invention provides salts of the compounds of general formula (I), supra, wherein:
R¹ is a hydrogen atom;
R² is a hydrogen atom;
R³ is a fluorine atom;
R⁴ is a hydrogen atom;
R⁵ is a hydrogen atom;
R⁶ is selected from a phenyl group and a heteroaryl group,
   wherein said phenyl or heteroaryl group is each optionally substituted with one or more substituents independently selected from a halogen atom, a C₁-C₆-alkyl group, a C₁-C₆-haloalkyl group, a C₁-C₆-alkoxy group, a (C₃-C₃-cycloalkyl)-(C₁-C₂-alkyl)-O— group, a (C₃-C₃-halocycloalkyl)-(C₁-C₂-alkyl)-O— group, a (phenyl)-(C₁-C₂-alkyl)-O— group, a C₁-C₆-haloalkoxy group, a C₃-C₃-cycloalkoxy group, a heterocycloalkyl group, a R⁷R⁸N— group, a (phenyl)-O— group and a heteroaryl group,
      wherein said heterocycloalkyl group is connected to the rest of the molecule via a carbon atom of said heterocycloalkyl group,
      wherein said heteroaryl or heterocycloalkyl group is each optionally substituted with one or more substituents independently selected from a C₁-C₃-alkyl group and a phenyl group;
R⁷ and R³ are each independently selected from a hydrogen atom, a C₁-C₆-alkyl group, a C₃-C₃-cycloalkyl group and a phenyl group,
   wherein said phenyl group is optionally substituted with one or more substituents independently selected from a halogen atom, a C₁-C₃-alkyl group, a C₁-C₃-haloalkyl group and a C₁-C₃-alkoxy group;
or R⁷ and R^{a} together with the nitrogen atom to which they are attached form a 4- to 7-membered nitrogen-containing heterocycloalkyl group, wherein said heterocycloalkyl group optionally contains one, two or three further heteroatoms independently selected from nitrogen, oxygen and sulfur and is optionally substituted with one or more substituents independently selected from a halogen atom, a C₁-C₃-alkyl group, a C₁-C₃-haloalkyl group, a C₁-C₃-alkoxy group and a phenyl group;
R⁹ is selected from a hydrogen atom, a C₁-C₃-alkyl group and a C₃-C₅-cycloalkyl group;
R^{a} and R^{b} together with the nitrogen atom to which they are attached form a 4-membered nitrogen-containing heterocycloalkyl group which is optionally substituted one time with a fluorine atom.

In accordance with further embodiments, the present invention provides sodium salts of the compounds of general formula (I), supra, wherein:
R¹ is a hydrogen atom;
R² is a hydrogen atom;
R³ is a fluorine atom;
R⁴ is a hydrogen atom;
R⁵ is a hydrogen atom;
R⁶ is selected from a phenyl group and a heteroaryl group,
   wherein said phenyl or heteroaryl group is each optionally substituted with one or more substituents independently selected from a halogen atom, a C₁-C₆-alkyl group, a C₁-C₆-haloalkyl group, a C₁-C₆-alkoxy group, a (C₃-C₃-cycloalkyl)-(C₁-C₂-alkyl)-O— group, a (C₃-C₃-halocycloalkyl)-(C₁-C₂-alkyl)-O— group, a (phenyl)-(C₁-C₂-alkyl)-O— group, a C₁-C₆-haloalkoxy group, a C₃-C₃-cycloalkoxy group, a heterocycloalkyl group, a R⁷R⁸N— group, a (phenyl)-O— group and a heteroaryl group,
      wherein said heterocycloalkyl group is connected to the rest of the molecule via a carbon atom of said heterocycloalkyl group,
      wherein said heteroaryl or heterocycloalkyl group is each optionally substituted with one or more substituents independently selected from a C₁-C₃-alkyl group and a phenyl group;
R⁷ and R⁸ are each independently selected from a hydrogen atom, a C₁-C₆-alkyl group, a C₃-C₃-cycloalkyl group and a phenyl group,
   wherein said phenyl group is optionally substituted with one or more substituents independently selected from a halogen atom, a C₁-C₃-alkyl group, a C₁-C₃-haloalkyl group and a C₁-C₃-alkoxy group;
or R⁷ and R⁸ together with the nitrogen atom to which they are attached form a 4- to 7-membered nitrogen-containing heterocycloalkyl group, wherein said heterocycloalkyl group optionally contains one, two or three further heteroatoms independently selected from nitrogen, oxygen and sulfur and is optionally substituted with one or more substituents independently selected from a halogen atom, a C₁-C₃-alkyl group, a C₁-C₃-haloalkyl group, a C₁-C₃-alkoxy group and a phenyl group;
R⁹ is selected from a hydrogen atom, a C₁-C₃-alkyl group and a C₃-C₆-cycloalkyl group;
R^{a} and R^{b} together with the nitrogen atom to which they are attached form a 4-membered nitrogen-containing heterocycloalkyl group which is optionally substituted one time with a fluorine atom.

The present invention provides the compounds of general formula (I) which are disclosed in the Example Section of this text, infra.

In some embodiments, the present invention includes compounds of general formula (I) selected from:
6-(Azetidin-1-yl)-N-(2-ethoxy-6-methoxybenzene-1-sulfonyl)-4-fluoro-1-benzofuran-2-carboxamide,
6-(Azetidin-1-yl)-N-(2-ethoxybenzene-1-sulfonyl)-4-fluoro-1-benzofuran-2-carboxamide,
6-(Azetidin-1-yl)-N-[2-(cyclopentyloxy)-5-ethylbenzene-1-sulfonyl]-4-fluoro-1-benzofuran-2-carboxamide,
6-(Azetidin-1-yl)-N-(2-{[butan-2-yl]oxy}-5-methylbenzene-1-sulfonyl)-4-fluoro-1-benzofuran-2-carboxamide,
6-(Azetidin-1-yl)-N-[2-(benzyloxy)-6-(cyclobutyloxy)benzene-1-sulfonyl]-4-fluoro-1-benzofuran-2-carboxamide,
6-(Azetidin-1-yl)-N-(2-ethoxy-5-methylbenzene-1-sulfonyl)-4-fluoro-1-benzofuran-2-carboxamide,
6-(Azetidin-1-yl)-N-[2-(benzyloxy)-6-ethoxybenzene-1-sulfonyl]-4-fluoro-1-benzofuran-2-carboxamide,
6-(Azetidin-1-yl)-4-fluoro-N-(7-methoxyquinoline-8-sulfonyl)-1-benzofuran-2-carboxamide,
6-(Azetidin-1-yl)-N-[2-(cyclopentyloxy)-5-methylbenzene-1-sulfonyl]-4-fluoro-1-benzofuran-2-carboxamide,
6-(Azetidin-1-yl)-N-(2-ethoxy-6-propoxybenzene-1-sulfonyl)-4-fluoro-1-benzofuran-2-carboxamide,
6-(Azetidin-1-yl)-N-[2-(dimethylamino)benzene-1-sulfonyl]-4-fluoro-1-benzofuran-2-carboxamide,
6-(Azetidin-1-yl)-N-(3,4-dihydro-2H-1-benzopyran-8-sulfonyl)-4-fluoro-1-benzofuran-2-carboxamide,
6-(Azetidin-1-yl)-4-fluoro-N-(2-{[-1,1,1-trifluoropropan-2-yl]oxy}benzene-1-sulfonyl)-1-benzofuran-2-carboxamide (rac),
6-(Azetidin-1-yl)-4-fluoro-N-(2-{[-1,1,1-trifluoropropan-2-yl]oxy}benzene-1-sulfonyl)-1-benzofuran-2-carboxamide (Enantiomer 1),
6-(Azetidin-1-yl)-4-fluoro-N-(2-{[-1,1,1-trifluoropropan-2-yl]oxy}benzene-1-sulfonyl)-1-benzofuran-2-carboxamide (Enantiomer 2),
6-(Azetidin-1-yl)-N-[2-(cyclobutyloxy)-6-fluorobenzene-1-sulfonyl]-4-fluoro-1-benzofuran-2-carboxamide,
6-(Azetidin-1-yl)-N-(2,6-dipropoxybenzene-1-sulfonyl)-4-fluoro-1-benzofuran-2-carboxamide,
6-(Azetidin-1-yl)-4-fluoro-N-[2-methyl-2,3-dihydro-1-benzofuran-7-sulfonyl]-1-benzofuran-2-carboxamide,
6-(Azetidin-1-yl)-N-([1,1′-biphenyl]-2-sulfonyl)-4-fluoro-1-benzofuran-2-carboxamide,
Sodium {[6-(azetidin-1-yl)-4-fluoro-1-benzofuran-2-yl]carbonyl}([biphenyl]-2-ylsulfonyl)azanide,
6-(Azetidin-1-yl)-N-(2,2-dimethyl-2,3-dihydro-1-benzofuran-7-sulfonyl)-4-fluoro-1-benzofuran-2-carboxamide,
6-(Azetidin-1-yl)-4-fluoro-N-(2-iodobenzene-1-sulfonyl)-1-benzofuran-2-carboxamide,
6-(Azetidin-1-yl)-N-[2-ethoxy-6-(propan-2-yl)benzene-1-sulfonyl]-4-fluoro-1-benzofuran-2-carboxamide,
6-(Azetidin-1-yl)-N-(2-cyclobutyl-6-fluorobenzene-1-sulfonyl)-4-fluoro-1-benzofuran-2-carboxamide,
6-(Azetidin-1-yl)-N-(2-ethoxy-6-methylbenzene-1-sulfonyl)-4-fluoro-1-benzofuran-2-carboxamide,
6-(Azetidin-1-yl)-N-([1,1′-biphenyl]-2-sulfonyl)-1-benzofuran-2-carboxamide,
6-(Azetidin-1-yl)-N-(3-chloro-6-methoxy-2-methylbenzene-1-sulfonyl)-4-fluoro-1-benzofuran-2-carboxamide,
6-(Azetidin-1-yl)-4-fluoro-N-{2-[(propan-2-yl)oxy]-6-(trifluoromethyl)benzene-1-sulfonyl}-1-benzofuran-2-carboxamide,
6-(Azetidin-1-yl)-N-{2-chloro-6-[(propan-2-yl)oxy]benzene-1-sulfonyl}-4-fluoro-1-benzofuran-2-carboxamide,
6-(Azetidin-1-yl)-N-[2,5-di(propan-2-yl)benzene-1-sulfonyl]-4-fluoro-1-benzofuran-2-carboxamide,
6-(Azetidin-1-yl)-4-fluoro-N-(2-propoxy-6-propylbenzene-1-sulfonyl)-1-benzofuran-2-carboxamide,
6-(Azetidin-1-yl)-4-fluoro-N-[2-methyl-5-(propan-2-yl)benzene-1-sulfonyl]-1-benzofuran-2-carboxamide,
6-(Azetidin-1-yl)-N-[2-(cyclopropylmethoxy)-6-ethylbenzene-1-sulfonyl]-4-fluoro-1-benzofuran-2-carboxamide,
6-(Azetidin-1-yl)-N-(5-ethyl-2-methylbenzene-1-sulfonyl)-4-fluoro-1-benzofuran-2-carboxamide,
6-(Azetidin-1-yl)-N-[2-(difluoromethoxy)-4-methylbenzene-1-sulfonyl]-4-fluoro-1-benzofuran-2-carboxamide,
6-(Azetidin-1-yl)-N-(4-ethyl-2-methylthiophene-3-sulfonyl)-4-fluoro-1-benzofuran-2-carboxamide,
6-(Azetidin-1-yl)-4-fluoro-N-[2-(3-methyl-1,2,4-oxadiazol-5-yl)benzene-1-sulfonyl]-1-benzofuran-2-carboxamide,
6-(Azetidin-1-yl)-N-[4-bromo-2-(propan-2-yl)benzene-1-sulfonyl]-4-fluoro-1-benzofuran-2-carboxamide,
6-(Azetidin-1-yl)-N-{2-bromo-6-[(propan-2-yl)oxy]benzene-1-sulfonyl}-4-fluoro-1-benzofuran-2-carboxamide,
6-(Azetidin-1-yl)-4-fluoro-N-(3-propylbenzene-1-sulfonyl)-1-benzofuran-2-carboxamide,
6-(Azetidin-1-yl)-4-fluoro-N-(1-methyl-1H-indole-7-sulfonyl)-1-benzofuran-2-carboxamide,
6-(Azetidin-1-yl)-4-fluoro-N-{2-fluoro-6-[(propan-2-yl)amino]benzene-1-sulfonyl}-1-benzofuran-2-carboxamide,
6-(Azetidin-1-yl)-4-fluoro-N-(2-fluoro-5-methylbenzene-1-sulfonyl)-1-benzofuran-2-carboxamide,
6-(Azetidin-1-yl)-N-[2-(3-ethyl-1,2,4-oxadiazol-5-yl)benzene-1-sulfonyl]-4-fluoro-1-benzofuran-2-carboxamide,
6-(Azetidin-1-yl)-N-[2-(benzenesulfonyl)benzene-1-sulfonyl]-4-fluoro-1-benzofuran-2-carboxamide,
6-(azetidin-1-yl)-N-[2-(ethylamino)benzene-1-sulfonyl]-4-fluoro-1-benzofuran-2-carboxamide,
6-(Azetidin-1-yl)-N-{2-[ethyl(methyl)carbamoyl]benzene-1-sulfonyl}-4-fluoro-1-benzofuran-2-carboxamide,
6-(Azetidin-1-yl)-4-fluoro-N-[3-(methoxymethyl)benzene-1-sulfonyl]-1-benzofuran-2-carboxamide,
6-(Azetidin-1-yl)-N-{5-chloro-2-[(propan-2-yl)amino]benzene-1-sulfonyl}-4-fluoro-1-benzofuran-2-carboxamide,
6-(Azetidin-1-yl)-N-[2-(cyclopropylamino)-6-fluorobenzene-1-sulfonyl]-4-fluoro-1-benzofuran-2-carboxamide,
6-(Azetidin-1-yl)-N-[2-(dimethylcarbamoyl)benzene-1-sulfonyl]-4-fluoro-1-benzofuran-2-carboxamide,
6-(Azetidin-1-yl)-4-fluoro-N-[2-(3,3,3-trifluoropropyl)benzene-1-sulfonyl]-1-benzofuran-2-carboxamide,
6-(Azetidin-1-yl)-4-fluoro-N-[2-methyl-6-(propan-2-yl)benzene-1-sulfonyl]-1-benzofuran-2-carboxamide,
6-(Azetidin-1-yl)-N-[2-chloro-5-(ethoxymethyl)benzene-1-sulfonyl]-4-fluoro-1-benzofuran-2-carboxamide,
6-(Azetidin-1-yl)-4-fluoro-N-{2-[(1E)-3,3,3-trifluoroprop-1-en-1-yl]benzene-1-sulfonyl}-1-benzofuran-2-carboxamide,
6-(Azetidin-1-yl)-4-fluoro-N-(quinoxaline-5-sulfonyl)-1-benzofuran-2-carboxamide,
Methyl (5-{[6-(azetidin-1-yl)-4-fluoro-1-benzofuran-2-carbonyl]sulfamoyl}naphthalen-2-yl)acetate,
6-(Azetidin-1-yl)-4-fluoro-N-[2-propyl-6-(trifluoromethyl)benzene-1-sulfonyl]-1-benzofuran-2-carboxamide,
6-(Azetidin-1-yl)-N-[2-chloro-5-(2-methoxyethoxy)benzene-1-sulfonyl]-4-fluoro-1-benzofuran-2-carboxamide,
6-(Azetidin-1-yl)-N-(2-chloro-5-methylpyridine-3-sulfonyl)-4-fluoro-1-benzofuran-2-carboxamide,
6-(Azetidin-1-yl)-N-(2-cyclopentyl-6-methylbenzene-1-sulfonyl)-4-fluoro-1-benzofuran-2-carboxamide,
2-{[6-(Azetidin-1-yl)-4-fluoro-1-benzofuran-2-carbonyl]sulfamoyl}phenyl
methyl(phenyl)carbamate,
6-(Azetidin-1-yl)-4-fluoro-N-[5-(hydroxymethyl)-2-(trifluoromethoxy)benzene-1-sulfonyl]-1-benzofuran-2-carboxamide,
6-(Azetidin-1-yl)-4-fluoro-N-(2-hydroxy-6-methylbenzene-1-sulfonyl)-1-benzofuran-2-carboxamide,
6-(Azetidin-1-yl)-4-fluoro-N-(5-methoxynaphthalene-1-sulfonyl)-1-benzofuran-2-carboxamide,
6-(Azetidin-1-yl)-N-[5-(dimethylamino)naphthalene-1-sulfonyl]-4-fluoro-1-benzofuran-2-carboxamide,
6-(Azetidin-1-yl)-4-fluoro-N-(3-methoxypyridine-2-sulfonyl)-1-benzofuran-2-carboxamide,
6-(Azetidin-1-yl)-N-[4-(2-cyanoethyl)benzene-1-sulfonyl]-4-fluoro-1-benzofuran-2-carboxamide,
6-(Azetidin-1-yl)-N-(3-bromopyridine-2-sulfonyl)-4-fluoro-1-benzofuran-2-carboxamide,
6-(Azetidin-1-yl)-4-fluoro-N-{2-[(3-methyl-1-phenyl-1H-pyrazol-5-yl)amino]benzene-1-sulfonyl}-1-benzofuran-2-carboxamide,
6-(Azetidin-1-yl)-4-fluoro-N-(3-hydroxynaphthalene-1-sulfonyl)-1-benzofuran-2-carboxamide,
6-(Azetidin-1-yl)-N-[2-chloro-5-(dimethylsulfamoyl)benzene-1-sulfonyl]-4-fluoro-1-benzofuran-2-carboxamide,
6-(Azetidin-1-yl)-4-fluoro-N-[2-(morpholine-4-carbonyl)benzene-1-sulfonyl]-1-benzofuran-2-carboxamide,
Methyl (2E)-3-(2-{[6-(azetidin-1-yl)-4-fluoro-1-benzofuran-2-carbonyl]sulfamoyl}phenyl)prop-2-enoate,
6-(Azetidin-1-yl)-N-(3-ethoxybenzene-1-sulfonyl)-4-fluoro-1-benzofuran-2-carboxamide,
2-{[6-(Azetidin-1-yl)-4-fluoro-1-benzofuran-2-carbonyl]sulfamoyl}phenyl diethylcarbamate,
6-(Azetidin-1-yl)-N-[2-(ethanesulfonyl)benzene-1-sulfonyl]-4-fluoro-1-benzofuran-2-carboxamide,
6-(azetidin-1-yl)-N-(5-tert-butyl-2-ethoxybenzene-1-sulfonyl)-4-fluoro-1-benzofuran-2-carboxamide,
6-(Azetidin-1-yl)-4-fluoro-N-[3-(propan-2-yl)pyridine-2-sulfonyl]-1-benzofuran-2-carboxamide,
6-(Azetidin-1-yl)-N-(2,2-dimethyl-3,4-dihydro-2H-1-benzopyran-8-sulfonyl)-4-fluoro-1-benzofuran-2-carboxamide,
6-(Azetidin-1-yl)-N-[2-(2,2-difluoroethoxy)-6-ethoxybenzene-1-sulfonyl]-4-fluoro-1-benzofuran-2-carboxamide,
6-(Azetidin-1-yl)-N-[5-bromo-2-(trifluoromethoxy)benzene-1-sulfonyl]-4-fluoro-1-benzofuran-2-carboxamide,
6-(Azetidin-1-yl)-N-(2-tert-butoxy-6-propoxybenzene-1-sulfonyl)-4-fluoro-1-benzofuran-2-carboxamide,
6-(Azetidin-1-yl)-N-{2-ethyl-6-[(propan-2-yl)oxy]benzene-1-sulfonyl}-4-fluoro-1-benzofuran-2-carboxamide,
6-(Azetidin-1-yl)-N-[2-(benzyloxy)-6-ethylbenzene-1-sulfonyl]-4-fluoro-1-benzofuran-2-carboxamide,
6-(Azetidin-1-yl)-4-fluoro-N-{2-methyl-6-[(propan-2-yl)oxy]benzene-1-sulfonyl}-1-benzofuran-2-carboxamide,
6-(azetidin-1-yl)-4-fluoro-N-(4-methylnaphthalene-1-sulfonyl)-1-benzofuran-2-carboxamide,
6-(Azetidin-1-yl)-N-(2,6-difluoro-3-methylbenzene-1-sulfonyl)-4-fluoro-1-benzofuran-2-carboxamide,
6-(Azetidin-1-yl)-4-fluoro-N-(5-propanamidonaphthalene-1-sulfonyl)-1-benzofuran-2-carboxamide,
Ethyl 8-{[6-(azetidin-1-yl)-4-fluoro-1-benzofuran-2-carbonyl]sulfamoyl}imidazo[1,2-a]pyridine-2-carboxylate,
6-(Azetidin-1-yl)-4-fluoro-N-[3-methyl-1-(propan-2-yl)-1H-pyrazole-4-sulfonyl]-1-benzofuran-2-carboxamide,
6-(Azetidin-1-yl)-N-{2-[(2,2-difluoroethyl)amino]-5-(trifluoromethyl)benzene-1-sulfonyl}-4-fluoro-1-benzofuran-2-carboxamide,
[3-sulfamoyl-4-(trifluoromethoxy)phenyl]methyl 6-(azetidin-1-yl)-4-fluoro-1-benzofuran-2-carboxylate,
6-(Azetidin-1-yl)-4-fluoro-N-(2-methylpyridine-3-sulfonyl)-1-benzofuran-2-carboxamide,
2-{[6-(Azetidin-1-yl)-4-fluoro-1-benzofuran-2-carbonyl]sulfamoyl}-N, N-dimethylpyridine-3-carboxamide,
6-(Azetidin-1-yl)-N-[2-(5,6-dihydro-1,4,2-dioxazin-3-yl)benzene-1-sulfonyl]-4-fluoro-1-benzofuran-2-carboxamide,
6-(Azetidin-1-yl)-N-[2-chloro-5-(dimethylamino)benzene-1-sulfonyl]-4-fluoro-1-benzofuran-2-carboxamide,
4-Sulfamoylnaphthalen-2-yl 6-(azetidin-1-yl)-4-fluoro-1-benzofuran-2-carboxylate,
N-(5-Acetyl-2-chlorobenzene-1-sulfonyl)-6-(azetidin-1-yl)-4-fluoro-1-benzofuran-2-carboxamide,
6-(Azetidin-1-yl)-N-[2-chloro-5-(1-hydroxyethyl)benzene-1-sulfonyl]-4-fluoro-1-benzofuran-2-carboxamide (rac),
Propan-2-yl 2-{[6-(azetidin-1-yl)-4-fluoro-1-benzofuran-2-carbonyl]sulfamoyl}benzoate,
Methyl 3-(2-{[6-(azetidin-1-yl)-4-fluoro-1-benzofuran-2-carbonyl]sulfamoyl}phenyl)propanoate,
6-(Azetidin-1-yl)-N-[2-chloro-5-(trifluoromethyl)pyridine-3-sulfonyl]-4-fluoro-1-benzofuran-2-carboxamide,
N-(2-amino-5-chloro-4,6-dimethylpyridine-3-sulfonyl)-6-(azetidin-1-yl)-4-fluoro-1-benzofuran-2-carboxamide,
Methyl {[1-{2-[(2-ethoxybenzene-1-sulfonyl)carbamoyl]-4-fluoro-1-benzofuran-6-yl}azetidin-2-yl]methyl}carbamate (rac),
6-(Azetidin-1-yl)-4-fluoro-N-(4-hydroxynaphthalene-1-sulfonyl)-1-benzofuran-2-carboxamide,
N-(2-Ethoxybenzene-1-sulfonyl)-6-(3-fluoroazetidin-1-yl)-1-benzofuran-2-carboxamide,
4-Fluoro-6-(3-fluoroazetidin-1-yl)-N-(5,6,7,8-tetrahydronaphthalene-1-sulfonyl)-1-benzofuran-2-carboxamide,
N-(2,3-dihydro-1,4-benzodioxine-5-sulfonyl)-4-fluoro-6-(3-fluoroazetidin-1-yl)-1-benzofuran-2-carboxamide,
4-Fluoro-6-(3-fluoroazetidin-1-yl)-N-(1,2,3,4-tetrahydroquinoline-8-sulfonyl)-1-benzofuran-2-carboxamide,
4-Fluoro-6-(3-fluoroazetidin-1-yl)-N-(quinoline-4-sulfonyl)-1-benzofuran-2-carboxamide,
4-Fluoro-6-(3-fluoroazetidin-1-yl)-N-(2-methyl-1,2,3,4-tetrahydroisoquinoline-5-sulfonyl)-1-benzofuran-2-carboxamide,
N-(5-tert-butyl-2-ethoxybenzene-1-sulfonyl)-4-fluoro-6-(3-fluoroazetidin-1-yl)-1-benzofuran-2-carboxamide,
N-[2-(dichloromethyl)benzene-1-sulfonyl]-4-fluoro-6-(3-fluoroazetidin-1-yl)-1-benzofuran-2-carboxamide,
4-Fluoro-6-(3-fluoroazetidin-1-yl)-N-[4-(2-methyl-1,3-thiazol-4-yl)benzene-1-sulfonyl]-1-benzofuran-2-carboxamide,
N-(2-Ethoxybenzene-1-sulfonyl)-6-(3-fluoro-3-methylazetidin-1-yl)-1-benzofuran-2-carboxamide,
N-(2-Ethoxybenzene-1-sulfonyl)-6-[3-(trifluoromethyl)azetidin-1-yl]-1-benzofuran-2-carboxamide,
6-(3,3-Difluoroazetidin-1-yl)-N-(2-ethoxybenzene-1-sulfonyl)-1-benzofuran-2-carboxamide,
N-([1,1′-biphenyl]-2-sulfonyl)-6-(3,3-difluoroazetidin-1-yl)-1-benzofuran-2-carboxamide,
N-(2-ethoxybenzene-1-sulfonyl)-6-(3-phenylazetidin-1-yl)-1-benzofuran-2-carboxamide,
6-(3-Ethoxyazetidin-1-yl)-N-(2-ethoxybenzene-1-sulfonyl)-1-benzofuran-2-carboxamide,
6-(5-Azaspiro[2.3]hexan-5-yl)-N-(2-ethoxybenzene-1-sulfonyl)-1-benzofuran-2-carboxamide,
6-(2-Azaspiro[3.3]heptan-2-yl)-N-(2-ethoxybenzene-1-sulfonyl)-1-benzofuran-2-carboxamide,
6-(3,3-dimethylazetidin-1-yl)-N-(2-ethoxybenzene-1-sulfonyl)-1-benzofuran-2-carboxamide,
N-(2-ethoxybenzene-1-sulfonyl)-6-(2-oxa-6-azaspiro[3.3]heptan-6-yl)-1-benzofuran-2-carboxamide,
Sodium [6-(azetidin-1-yl)-4-fluoro-1-benzofuran-2-carbonyl](2-ethoxybenzene-1-sulfonyl)azanide,
6-(Azetidin-1-yl)-N-(5-tert-butyl-2-methoxybenzene-1-sulfonyl)-4-fluoro-1-benzofuran-2-carboxamide,
6-(Azetidin-1-yl)-N-[2-ethoxy-5-(propan-2-yl)benzene-1-sulfonyl]-4-fluoro-1-benzofuran-2-carboxamide,
6-(Azetidin-1-yl)-4-fluoro-N-{2-[(propan-2-yl)oxy]benzene-1-sulfonyl}-1-benzofuran-2-carboxamide,
6-(Azetidin-1-yl)-4-fluoro-N-(2-methylquinoline-8-sulfonyl)-1-benzofuran-2-carboxamide,
6-(Azetidin-1-yl)-N-[2-(cyclopropyloxy)benzene-1-sulfonyl]-4-fluoro-1-benzofuran-2-carboxamide,
6-(Azetidin-1-yl)-N-(2-chloroquinoline-8-sulfonyl)-4-fluoro-1-benzofuran-2-carboxamide,
6-(Azetidin-1-yl)-4-fluoro-N-(2-propoxybenzene-1-sulfonyl)-1-benzofuran-2-carboxamide,
6-(Azetidin-1-yl)-4-fluoro-N-{5-methyl-2-[(propan-2-yl)oxy]benzene-1-sulfonyl}-1-benzofuran-2-carboxamide,
6-(Azetidin-1-yl)-N-(5-bromo-2-ethoxybenzene-1-sulfonyl)-4-fluoro-1-benzofuran-2-carboxamide,
6-(azetidin-1-yl)-N-(2-ethyl-6-methoxybenzene-1-sulfonyl)-4-fluoro-1-benzofuran-2-carboxamide,
6-(Azetidin-1-yl)-4-fluoro-N-[2-methoxy-5-(morpholin-4-yl)benzene-1-sulfonyl]-1-benzofuran-2-carboxamide,
6-(Azetidin-1-yl)-4-fluoro-N-[2-(trifluoromethoxy)benzene-1-sulfonyl]-1-benzofuran-2-carboxamide,
6-(Azetidin-1-yl)-N-(5-bromo-2-methoxybenzene-1-sulfonyl)-4-fluoro-1-benzofuran-2-carboxamide,
6-(Azetidin-1-yl)-4-fluoro-N-(2,2,3,3-tetrafluoro-2,3-dihydro-1,4-benzodioxine-5-sulfonyl)-1-benzofuran-2-carboxamide,
6-(Azetidin-1-yl)-N-(1,1-dioxo-1,2,3,4-tetrahydro-1-benzothiopyran-8-sulfonyl)-4-fluoro-1-benzofuran-2-carboxamide,
6-(Azetidin-1-yl)-N-(5-chloro-2,2-difluoro-2H-1,3-benzodioxole-4-sulfonyl)-4-fluoro-1-benzofuran-2-carboxamide,
6-(Azetidin-1-yl)-N-(1-ethyl-1H-pyrazolo[3,4-b]pyridine-3-sulfonyl)-4-fluoro-1-benzofuran-2-carboxamide,
6-(Azetidin-1-yl)-4-fluoro-N-[1-methyl-6-(trifluoromethyl)-1H-pyrazolo[3,4-b]pyridine-3-sulfonyl]-1-benzofuran-2-carboxamide,
6-(Azetidin-1-yl)-N-(1,6-dimethyl-1H-pyrazolo[3,4-b]pyridine-3-sulfonyl)-4-fluoro-1-benzofuran-2-carboxamide,
6-(Azetidin-1-yl)-N-(1,4-dimethyl-1H-pyrazolo[3,4-b]pyridine-3-sulfonyl)-4-fluoro-1-benzofuran-2-carboxamide,
6-(Azetidin-1-yl)-N-(2-chloro-6-propylimidazo[1,2-b]pyridazine-3-sulfonyl)-4-fluoro-1-benzofuran-2-carboxamide,
6-(Azetidin-1-yl)-N-[2-(ethylsulfanyl)imidazo[1,2-a]pyridine-3-sulfonyl]-4-fluoro-1-benzofuran-2-carboxamide,
6-(Azetidin-1-yl)-4-fluoro-N-[2-(methylsulfanyl)imidazo[1,2-a]pyridine-3-sulfonyl]-1-benzofuran-2-carboxamide,
N-(5-tert-butyl-2-methoxybenzene-1-sulfonyl)-4-fluoro-6-(3-fluoroazetidin-1-yl)-1-benzofuran-2-carboxamide,
N-[2-ethoxy-5-(propan-2-yl)benzene-1-sulfonyl]-4-fluoro-6-(3-fluoroazetidin-1-yl)-1-benzofuran-2-carboxamide,
4-Fluoro-6-(3-fluoroazetidin-1-yl)-N-(2-methylquinoline-8-sulfonyl)-1-benzofuran-2-carboxamide,
N-(2-ethoxy-5-ethylbenzene-1-sulfonyl)-4-fluoro-6-(3-fluoroazetidin-1-yl)-1-benzofuran-2-carboxamide,
N-(2-Chloroquinoline-8-sulfonyl)-4-fluoro-6-(3-fluoroazetidin-1-yl)-1-benzofuran-2-carboxamide,
4-Fluoro-6-(3-fluoroazetidin-1-yl)-N-{2-[(propan-2-yl)oxy]benzene-1-sulfonyl}-1-benzofuran-2-carboxamide,
6-(Azetidin-1-yl)-4-fluoro-N-(2-methoxy-4-methylbenzene-1-sulfonyl)-1-benzofuran-2-carboxamide,
4-Fluoro-6-(3-fluoroazetidin-1-yl)-N-{5-methyl-2-[(propan-2-yl)oxy]benzene-1-sulfonyl}-1-benzofuran-2-carboxamide,
4-Fluoro-6-(3-fluoroazetidin-1-yl)-N-(2-methoxy-4-methylbenzene-1-sulfonyl)-1-benzofuran-2-carboxamide,
4-Fluoro-6-(3-fluoroazetidin-1-yl)-N-(2,2,3,3-tetrafluoro-2,3-dihydro-1,4-benzodioxine-5-sulfonyl)-1-benzofuran-2-carboxamide,
6-[2-(Difluoromethyl)azetidin-1-yl]-4-fluoro-N-(2-methylquinoline-8-sulfonyl)-1-benzofuran-2-carboxamide (rac),
4-Fluoro-6-[2-(1-hydroxy-1-methyl-ethyl)azetidin-1-yl]-N-[(2-methyl-8-quinolyl)sulfonyl]benzofuran-2-carboxamide (rac),
N-(2-ethoxybenzene-1-sulfonyl)-4-fluoro-6-[2-(2-hydroxypropan-2-yl)azetidin-1-yl]-1-benzofuran-2-carboxamide (rac),
6-[2-(Difluoromethyl)azetidin-1-yl]-N-(2-ethoxybenzene-1-sulfonyl)-4-fluoro-1-benzofuran-2-carboxamide (rac),
N-{2,6-difluoro-4-[methyl(propan-2-yl)amino]benzene-1-sulfonyl}-4-fluoro-6-(3-fluoroazetidin-1-yl)-1-benzofuran-2-carboxamide,
N-{2,4-difluoro-6-[methyl(propan-2-yl)amino]benzene-1-sulfonyl}-4-fluoro-6-(3-fluoroazetidin-1-yl)-1-benzofuran-2-carboxamide,
N-{2,3-difluoro-4-[methyl(propan-2-yl)amino]benzene-1-sulfonyl}-4-fluoro-6-(3-fluoroazetidin-1-yl)-1-benzofuran-2-carboxamide,
6-(Azetidin-1-yl)-N-[2-(ethanesulfonyl)imidazo[1,2-a]pyridine-3-sulfonyl]-4-fluoro-1-benzofuran-2-carboxamide,
6-(Azetidin-1-yl)-N-(6,8-dichloroimidazo[1,2-a]pyridine-3-sulfonyl)-4-fluoro-1-benzofuran-2-carboxamide,
1-{2-[(2-Ethoxybenzene-1-sulfonyl)carbamoyl]-4-fluoro-1-benzofuran-6-yl}azetidine-2-carboxamide (rac),
6-(azetidin-1-yl)-N-{5-bromo-2-[(propan-2-yl)oxy]benzene-1-sulfonyl}-4-fluoro-1-benzofuran-2-carboxamide,
6-(Azetidin-1-yl)-N-(2-ethylquinoline-8-sulfonyl)-4-fluoro-1-benzofuran-2-carboxamide,
6-(Azetidin-1-yl)-N-(2,3-dihydro-1,4-benzodioxine-5-sulfonyl)-4-fluoro-1-benzofuran-2-carboxamide,
6-(Azetidin-1-yl)-4-fluoro-N-(7-methylquinoline-8-sulfonyl)-1-benzofuran-2-carboxamide,
6-(Azetidin-1-yl)-N-(4-bromo-2-methoxybenzene-1-sulfonyl)-4-fluoro-1-benzofuran-2-carboxamide,
6-(Azetidin-1-yl)-4-fluoro-N-(6-methylquinoline-8-sulfonyl)-1-benzofuran-2-carboxamide,
6-(Azetidin-1-yl)-4-fluoro-N-(1H-indole-3-sulfonyl)-1-benzofuran-2-carboxamide,
6-(Azetidin-1-yl)-N-(5-bromo-2,3-dihydro-1-benzofuran-7-sulfonyl)-4-fluoro-1-benzofuran-2-carboxamide,
6-(Azetidin-1-yl)-N-(1-benzothiophene-3-sulfonyl)-4-fluoro-1-benzofuran-2-carboxamide,
6-(Azetidin-1-yl)-4-fluoro-N-(2-methyl-2H-benzotriazole-4-sulfonyl)-1-benzofuran-2-carboxamide,
6-(Azetidin-1-yl)-4-fluoro-N-(4-methoxy-5,6,7,8-tetrahydronaphthalene-1-sulfonyl)-1-benzofuran-2-carboxamide,
6-(Azetidin-1-yl)-N-(2,1,3-benzothiadiazole-4-sulfonyl)-4-fluoro-1-benzofuran-2-carboxamide,
6-(Azetidin-1-yl)-4-fluoro-N-(5-methoxy-1,2,3-benzothiadiazole-4-sulfonyl)-1-benzofuran-2-carboxamide,
6-(Azetidin-1-yl)-N-(3,4-dichloro-2-methoxybenzene-1-sulfonyl)-4-fluoro-1-benzofuran-2-carboxamide,
6-(Azetidin-1-yl)-N-(6,7-dihydro-5H-pyrazolo[5,1-b][1,3]oxazine-3-sulfonyl)-4-fluoro-1-benzofuran-2-carboxamide,
N-(4-Aacetamidonaphthalene-1-sulfonyl)-6-(azetidin-1-yl)-4-fluoro-1-benzofuran-2-carboxamide,
6-(Azetidin-1-yl)-4-fluoro-N-(imidazo[1,2-a]pyridine-3-sulfonyl)-1-benzofuran-2-carboxamide,
6-(Azetidin-1-yl)-4-fluoro-N-(isoquinoline-5-sulfonyl)-1-benzofuran-2-carboxamide,
6-(Azetidin-1-yl)-4-fluoro-N-[5-(morpholin-4-yl)naphthalene-1-sulfonyl]-1-benzofuran-2-carboxamide,
6-(Azetidin-1-yl)-N-(5-tert-butyl-2,3-dimethylbenzene-1-sulfonyl)-4-fluoro-1-benzofuran-2-carboxamide,
6-(Azetidin-1-yl)-N-(3,3-dimethyl-2,3-dihydro-1-benzofuran-5-sulfonyl)-4-fluoro-1-benzofuran-2-carboxamide,
6-(Azetidin-1-yl)-4-fluoro-N-(8-methylisoquinoline-5-sulfonyl)-1-benzofuran-2-carboxamide,
Methyl (5-{[6-(azetidin-1-yl)-4-fluoro-1-benzofuran-2-carbonyl]sulfamoyl}naphthalen-1-yl)carbamate,
6-(Azetidin-1-yl)-4-fluoro-N-(4-methylisoquinoline-5-sulfonyl)-1-benzofuran-2-carboxamide,
6-(Azetidin-1-yl)-N-[5-(3,5-dimethyl-1,2-oxazol-4-yl)-2-methoxybenzene-1-sulfonyl]-4-fluoro-1-benzofuran-2-carboxamide,
6-(Azetidin-1-yl)-N-[5-(1,3-dimethyl-1H-pyrazol-4-yl)-2-methoxybenzene-1-sulfonyl]-4-fluoro-1-benzofuran-2-carboxamide,
6-(azetidin-1-yl)-N-[5-(3,6-dihydro-2H-pyran-4-yl)-2-methoxybenzene-1-sulfonyl]-4-fluoro-1-benzofuran-2-carboxamide,
tert-butyl 4-(3-{[6-(azetidin-1-yl)-4-fluoro-1-benzofuran-2-carbonyl]sulfamoyl}-4-methoxyphenyl)-3,6-dihydropyridine-1(2H)-carboxylate,
6-(Azetidin-1-yl)-4-fluoro-N-(3′-fluoro-4-methoxy[1,1′-biphenyl]-3-sulfonyl)-1-benzofuran-2-carboxamide,
6-(Azetidin-1-yl)-4-fluoro-N-[2-methoxy-5-(1-methyl-1H-pyrazol-4-yl)benzene-1-sulfonyl]-1-benzofuran-2-carboxamide,
6-(Azetidin-1-yl)-4-fluoro-N-[2-methoxy-5-(pyridin-3-yl)benzene-1-sulfonyl]-1-benzofuran-2-carboxamide,
6-(Azetidin-1-yl)-4-fluoro-N-[2-methoxy-5-(2-phenylpyrrolidin-1-yl)benzene-1-sulfonyl]-1-benzofuran-2-carboxamide (rac),
6-(Azetidin-1-yl)-4-fluoro-N-[2-methoxy-5-(4-methylpiperazin-1-yl)benzene-1-sulfonyl]-1-benzofuran-2-carboxamide,
6-(Azetidin-1-yl)-4-fluoro-N-[2-methoxy-5-(1-methyl-1,2,3,6-tetrahydropyridin-4-yl)benzene-1-sulfonyl]-1-benzofuran-2-carboxamide,
6-(Azetidin-1-yl)-4-fluoro-N-(2,2,5,6-tetramethyl-2,3-dihydro-1-benzofuran-7-sulfonyl)-1-benzofuran-2-carboxamide,
6-(Azetidin-1-yl)-4-fluoro-N-[2-methoxy-5-(prop-1-en-2-yl)benzene-1-sulfonyl]-1-benzofuran-2-carboxamide,
6-(azetidin-1-yl)-N-(2-ethoxy-3-fluoro-phenyl)sulfonyl-4-fluoro-benzofuran-2-carboxamide,
6-(Azetidin-1-yl)-N-[(5-sec-butyl-2-ethoxyphenyl)sulfonyl]-4-fluoro-1-benzofuran-2-carboxamide (rac),
6-(Azetidin-1-yl)-N-(2-ethoxy-4-fluoro-phenyl)sulfonyl-4-fluoro-benzofuran-2-carboxamide,
6-(azetidin-1-yl)-N-[2-ethoxy-5-(trifluoromethyl)phenyl]sulfonyl-4-fluoro-benzofuran-2-carboxamide,
6-(Azetidin-1-yl)-N-(2-ethoxy-5-fluoro-phenyl)sulfonyl-4-fluoro-benzofuran-2-carboxamide,
6-(Azetidin-1-yl)-N-(2-ethoxy-5-phenoxy-phenyl)sulfonyl-4-fluoro-benzofuran-2-carboxamide,
6-(Azetidin-1-yl)-N-[2-ethoxy-5-(trifluoromethoxy)phenyl]sulfonyl-4-fluoro-benzofuran-2-carboxamide,
6-(azetidin-1-yl)-N-(2-benzyloxy-5-isopropyl-phenyl)sulfonyl-4-fluoro-benzofuran-2-carboxamide,
6-(azetidin-1-yl)-N-[2-(cyclopropylmethoxy)-5-isopropyl-phenyl]sulfonyl-4-fluoro-benzofuran-2-carboxamide,
6-(azetidin-1-yl)-4-fluoro-N-[5-isopropyl-2-(2,2,2-trifluoroethoxy)phenyl]sulfonyl-benzofuran-2-carboxamide,
N-(benzenesulfonyl)-6-(2,2-dimethylazetidin-1-yl)-4-fluoro-benzofuran-2-carboxamide,
6-(2,2-Dimethylazetidin-1-yl)-4-fluoro-N-(2-phenylphenyl)sulfonyl-benzofuran-2-carboxamide,
6-(2,2-Dimethylazetidin-1-yl)-4-fluoro-N-[(2-methyl-8-quinolyl)sulfonyl]benzofuran-2-carboxamide,
6-(2,2-Dimethylazetidin-1-yl)-N-(2-ethoxyphenyl)sulfonyl-4-fluoro-benzofuran-2-carboxamide,
N-(Benzenesulfonyl)-4-fluoro-6-(2-methylazetidin-1-yl)benzofuran-2-carboxamide (rac),
4-Fluoro-6-(2-methylazetidin-1-yl)-N-[(2-methyl-8-quinolyl)sulfonyl]benzofuran-2-carboxamide (rac),
4-Fluoro-6-(2-methylazetidin-1-yl)-N-(2-phenylphenyl)sulfonyl-benzofuran-2-carboxamide (rac),
N-(2-Ethoxyphenyl)sulfonyl-4-fluoro-6-(2-methylazetidin-1-yl)benzofuran-2-carboxamide (rac),
6-(Azetidin-1-yl)-N-[(5-(tert-butyl)-2-cyclopropoxyphenyl)sulfonyl]-4-fluorobenzofuran-2-carboxamide,
6-(Azetidin-1-yl)-N-[(2-(benzyloxy)-5-(tert-butyl)phenyl]sulfonyl)-4-fluorobenzofuran-2-carboxamide,
6-(Azetidin-1-yl)-N-[(5-(tert-butyl)-2-(cyclopropylmethoxy)phenyl)sulfonyl]-4-fluorobenzofuran-2-carboxamide,
6-(Azetidin-1-yl)-N-[(5-(tert-butyl)-2-(2,2,2-trifluoroethoxy)phenyl)sulfonyl]-4-fluorobenzofuran-2-carboxamide,
6-(Azetidin-1-yl)-N-[(5-(tert-butyl)-2-cyclobutoxyphenyl)sulfonyl]-4-fluorobenzofuran-2-carboxamide,
6-(Azetidin-1-yl)-N-[(5-(tert-butyl)-2-isopropoxyphenyl)sulfonyl]-4-fluorobenzofuran-2-carboxamide,
6-(Azetidin-1-yl)-N-[(2-cyclobutoxy-5-isopropylphenyl)sulfonyl]-4-fluorobenzofuran-2-carboxamide,
6-(Azetidin-1-yl)-N-((2-((2,2-difluorocyclopropyl)methoxy)-5-isopropylphenyl)sulfonyl)-4-fluorobenzofuran-2-carboxamide (rac),
6-(Azetidin-1-yl)-N-((2-ethoxy-4,5-difluorophenyl)sulfonyl)-4-fluorobenzofuran-2-carboxamide,
6-(Azetidin-1-yl)-4-fluoro-N-[(2-methoxy-5-(methoxymethyl)phenyl)sulfonyl]benzofuran-2-carboxamide,
6-(Azetidin-1-yl)-N-[(5-cyclopropyl-2-methoxyphenyl)sulfonyl]-4-fluorobenzofuran-2-carboxamide,
6-(Azetidin-1-yl)-N-((5-cyclopropyl-2-ethoxyphenyl)sulfonyl)-4-fluorobenzofuran-2-carboxamide,
6-(Azetidin-1-yl)-4-fluoro-N-[(2-methoxy-5-(2-oxopropyl)phenyl)sulfonyl]benzofuran-2-carboxamide and
tert-butyl 4-((2-(N-(6-(azetidin-1-yl)-4-fluorobenzofuran-2-carbonyl)sulfamoyl)-4-(tert-butyl)phenoxy)methyl)piperidine-1-carboxylate.
In some embodiments, the present invention includes compounds of general formula (I) selected from:
6-(Azetidin-1-yl)-N-(2-ethoxy-6-methoxybenzene-1-sulfonyl)-4-fluoro-1-benzofuran-2-carboxamide,
6-(Azetidin-1-yl)-N-(2-ethoxybenzene-1-sulfonyl)-4-fluoro-1-benzofuran-2-carboxamide,
6-(Azetidin-1-yl)-N-[2-(cyclopentyloxy)-5-ethylbenzene-1-sulfonyl]-4-fluoro-1-benzofuran-2-carboxamide,
6-(Azetidin-1-yl)-N-(2-{[butan-2-yl]oxy}-5-methylbenzene-1-sulfonyl)-4-fluoro-1-benzofuran-2-carboxamide,
6-(Azetidin-1-yl)-N-[2-(benzyloxy)-6-(cyclobutyloxy)benzene-1-sulfonyl]-4-fluoro-1-benzofuran-2-carboxamide,
6-(Azetidin-1-yl)-N-(2-ethoxy-5-methylbenzene-1-sulfonyl)-4-fluoro-1-benzofuran-2-carboxamide,
6-(Azetidin-1-yl)-N-[2-(benzyloxy)-6-ethoxybenzene-1-sulfonyl]-4-fluoro-1-benzofuran-2-carboxamide,
6-(Azetidin-1-yl)-4-fluoro-N-(7-methoxyquinoline-8-sulfonyl)-1-benzofuran-2-carboxamide,
6-(Azetidin-1-yl)-N-[2-(cyclopentyloxy)-5-methylbenzene-1-sulfonyl]-4-fluoro-1-benzofuran-2-carboxamide,
6-(Azetidin-1-yl)-N-(2-ethoxy-6-propoxybenzene-1-sulfonyl)-4-fluoro-1-benzofuran-2-carboxamide,
6-(Azetidin-1-yl)-4-fluoro-N-(2-{[-1,1,1-trifluoropropan-2-yl]oxy}benzene-1-sulfonyl)-1-benzofuran-2-carboxamide (rac),
6-(Azetidin-1-yl)-4-fluoro-N-(2-{[-1,1,1-trifluoropropan-2-yl]oxy}benzene-1-sulfonyl)-1-benzofuran-2-carboxamide (Enantiomer 1),
6-(Azetidin-1-yl)-4-fluoro-N-(2-{[-1,1,1-trifluoropropan-2-yl]oxy}benzene-1-sulfonyl)-1-benzofuran-2-carboxamide (Enantiomer 2),
6-(Azetidin-1-yl)-N-[2-(cyclobutyloxy)-6-fluorobenzene-1-sulfonyl]-4-fluoro-1-benzofuran-2-carboxamide,
6-(Azetidin-1-yl)-N-(2,6-dipropoxybenzene-1-sulfonyl)-4-fluoro-1-benzofuran-2-carboxamide,
6-(Azetidin-1-yl)-4-fluoro-N-[2-methyl-2,3-dihydro-1-benzofuran-7-sulfonyl]-1-benzofuran-2-carboxamide,
6-(Azetidin-1-yl)-N-([1,1′-biphenyl]-2-sulfonyl)-4-fluoro-1-benzofuran-2-carboxamide,
Sodium {[6-(azetidin-1-yl)-4-fluoro-1-benzofuran-2-yl]carbonyl}([biphenyl]-2-ylsulfonyl)azanide,
6-(Azetidin-1-yl)-N-(2,2-dimethyl-2,3-dihydro-1-benzofuran-7-sulfonyl)-4-fluoro-1-benzofuran-2-carboxamide,
6-(Azetidin-1-yl)-N-[2-ethoxy-6-(propan-2-yl)benzene-1-sulfonyl]-4-fluoro-1-benzofuran-2-carboxamide,
6-(Azetidin-1-yl)-N-(2-ethoxy-6-methylbenzene-1-sulfonyl)-4-fluoro-1-benzofuran-2-carboxamide,
6-(Azetidin-1-yl)-N-([1,1′-biphenyl]-2-sulfonyl)-1-benzofuran-2-carboxamide,
6-(Azetidin-1-yl)-N-(3-chloro-6-methoxy-2-methylbenzene-1-sulfonyl)-4-fluoro-1-benzofuran-2-carboxamide,
6-(Azetidin-1-yl)-N-{2-chloro-6-[(propan-2-yl)oxy]benzene-1-sulfonyl}-4-fluoro-1-benzofuran-2-carboxamide,
6-(Azetidin-1-yl)-N-[2,5-di(propan-2-yl)benzene-1-sulfonyl]-4-fluoro-1-benzofuran-2-carboxamide,
6-(Azetidin-1-yl)-4-fluoro-N-(2-propoxy-6-propylbenzene-1-sulfonyl)-1-benzofuran-2-carboxamide,
6-(Azetidin-1-yl)-4-fluoro-N-[2-methyl-5-(propan-2-yl)benzene-1-sulfonyl]-1-benzofuran-2-carboxamide,
6-(Azetidin-1-yl)-N-[2-(cyclopropylmethoxy)-6-ethylbenzene-1-sulfonyl]-4-fluoro-1-benzofuran-2-carboxamide,
6-(Azetidin-1-yl)-4-fluoro-N-[2-(3-methyl-1,2,4-oxadiazol-5-yl)benzene-1-sulfonyl]-1-benzofuran-2-carboxamide,
6-(Azetidin-1-yl)-N-{2-bromo-6-[(propan-2-yl)oxy]benzene-1-sulfonyl}-4-fluoro-1-benzofuran-2-carboxamide,
6-(Azetidin-1-yl)-4-fluoro-N-[2-(3,3,3-trifluoropropyl)benzene-1-sulfonyl]-1-benzofuran-2-carboxamide,
6-(Azetidin-1-yl)-4-fluoro-N-[2-methyl-6-(propan-2-yl)benzene-1-sulfonyl]-1-benzofuran-2-carboxamide,
6-(Azetidin-1-yl)-N-[2-chloro-5-(ethoxymethyl)benzene-1-sulfonyl]-4-fluoro-1-benzofuran-2-carboxamide,
6-(Azetidin-1-yl)-4-fluoro-N-{2-[(1E)-3,3,3-trifluoroprop-1-en-1-yl]benzene-1-sulfonyl}-1-benzofuran-2-carboxamide,
6-(Azetidin-1-yl)-4-fluoro-N-(quinoxaline-5-sulfonyl)-1-benzofuran-2-carboxamide,
Methyl (5-{[6-(azetidin-1-yl)-4-fluoro-1-benzofuran-2-carbonyl]sulfamoyl}naphthalen-2-yl)acetate,
6-(Azetidin-1-yl)-4-fluoro-N-[2-(morpholine-4-carbonyl)benzene-1-sulfonyl]-1-benzofuran-2-carboxamide,
Methyl (2E)-3-(2-{[6-(azetidin-1-yl)-4-fluoro-1-benzofuran-2-carbonyl]sulfamoyl}phenyl)prop-2-enoate,
6-(Azetidin-1-yl)-N-(3-ethoxybenzene-1-sulfonyl)-4-fluoro-1-benzofuran-2-carboxamide,
2-{[6-(Azetidin-1-yl)-4-fluoro-1-benzofuran-2-carbonyl]sulfamoyl}phenyl diethylcarbamate,
6-(Azetidin-1-yl)-N-[2-(ethanesulfonyl)benzene-1-sulfonyl]-4-fluoro-1-benzofuran-2-carboxamide,
6-(azetidin-1-yl)-N-(5-tert-butyl-2-ethoxybenzene-1-sulfonyl)-4-fluoro-1-benzofuran-2-carboxamide,
6-(Azetidin-1-yl)-4-fluoro-N-[3-(propan-2-yl)pyridine-2-sulfonyl]-1-benzofuran-2-carboxamide,
6-(Azetidin-1-yl)-N-(2,2-dimethyl-3,4-dihydro-2H-1-benzopyran-8-sulfonyl)-4-fluoro-1-benzofuran-2-carboxamide,
6-(Azetidin-1-yl)-N-[2-(2,2-difluoroethoxy)-6-ethoxybenzene-1-sulfonyl]-4-fluoro-1-benzofuran-2-carboxamide,
6-(Azetidin-1-yl)-N-[5-bromo-2-(trifluoromethoxy)benzene-1-sulfonyl]-4-fluoro-1-benzofuran-2-carboxamide,
6-(Azetidin-1-yl)-N-(2-tert-butoxy-6-propoxybenzene-1-sulfonyl)-4-fluoro-1-benzofuran-2-carboxamide,
6-(Azetidin-1-yl)-N-[2-(benzyloxy)-6-ethylbenzene-1-sulfonyl]-4-fluoro-1-benzofuran-2-carboxamide,
6-(Azetidin-1-yl)-4-fluoro-N-{2-methyl-6-[(propan-2-yl)oxy]benzene-1-sulfonyl}-1-benzofuran-2-carboxamide,
Propan-2-yl 2-{[6-(azetidin-1-yl)-4-fluoro-1-benzofuran-2-carbonyl]sulfamoyl}benzoate,
N-(2-Ethoxybenzene-1-sulfonyl)-6-(3-fluoroazetidin-1-yl)-1-benzofuran-2-carboxamide,
N-(5-tert-butyl-2-ethoxybenzene-1-sulfonyl)-4-fluoro-6-(3-fluoroazetidin-1-yl)-1-benzofuran-2-carboxamide,
Sodium [6-(azetidin-1-yl)-4-fluoro-1-benzofuran-2-carbonyl](2-ethoxybenzene-1-sulfonyl)azanide,
6-(Azetidin-1-yl)-N-(5-tert-butyl-2-methoxybenzene-1-sulfonyl)-4-fluoro-1-benzofuran-2-carboxamide,
6-(Azetidin-1-yl)-N-[2-ethoxy-5-(propan-2-yl)benzene-1-sulfonyl]-4-fluoro-1-benzofuran-2-carboxamide,
6-(Azetidin-1-yl)-4-fluoro-N-{2-[(propan-2-yl)oxy]benzene-1-sulfonyl}-1-benzofuran-2-carboxamide,
6-(Azetidin-1-yl)-4-fluoro-N-(2-methylquinoline-8-sulfonyl)-1-benzofuran-2-carboxamide
6-(Azetidin-1-yl)-N-[2-(cyclopropyloxy)benzene-1-sulfonyl]-4-fluoro-1-benzofuran-2-carboxamide,
6-(Azetidin-1-yl)-N-(2-chloroquinoline-8-sulfonyl)-4-fluoro-1-benzofuran-2-carboxamide
6-(Azetidin-1-yl)-4-fluoro-N-(2-propoxybenzene-1-sulfonyl)-1-benzofuran-2-carboxamide,
6-(Azetidin-1-yl)-4-fluoro-N-{5-methyl-2-[(propan-2-yl)oxy]benzene-1-sulfonyl}-1-benzofuran-2-carboxamide,
6-(Azetidin-1-yl)-N-(5-bromo-2-ethoxybenzene-1-sulfonyl)-4-fluoro-1-benzofuran-2-carboxamide,
6-(azetidin-1-yl)-N-(2-ethyl-6-methoxybenzene-1-sulfonyl)-4-fluoro-1-benzofuran-2-carboxamide,
6-(Azetidin-1-yl)-4-fluoro-N-[2-methoxy-5-(morpholin-4-yl)benzene-1-sulfonyl]-1-benzofuran-2-carboxamide,
6-(Azetidin-1-yl)-4-fluoro-N-[2-(trifluoromethoxy)benzene-1-sulfonyl]-1-benzofuran-2-carboxamide,
6-(Azetidin-1-yl)-N-(5-bromo-2-methoxybenzene-1-sulfonyl)-4-fluoro-1-benzofuran-2-carboxamide,
6-(Azetidin-1-yl)-N-(1,4-dimethyl-1H-pyrazolo[3,4-b]pyridine-3-sulfonyl)-4-fluoro-1-benzofuran-2-carboxamide,
6-(Azetidin-1-yl)-N-(2-chloro-6-propylimidazo[1,2-b]pyridazine-3-sulfonyl)-4-fluoro-1-benzofuran-2-carboxamide,
6-(Azetidin-1-yl)-N-[2-(ethylsulfanyl)imidazo[1,2-a]pyridine-3-sulfonyl]-4-fluoro-1-benzofuran-2-carboxamide,
N-(5-tert-butyl-2-methoxybenzene-1-sulfonyl)-4-fluoro-6-(3-fluoroazetidin-1-yl)-1-benzofuran-2-carboxamide,
N-[2-ethoxy-5-(propan-2-yl)benzene-1-sulfonyl]-4-fluoro-6-(3-fluoroazetidin-1-yl)-1-benzofuran-2-carboxamide,
4-Fluoro-6-(3-fluoroazetidin-1-yl)-N-(2-methylquinoline-8-sulfonyl)-1-benzofuran-2-carboxamide,
N-(2-ethoxy-5-ethylbenzene-1-sulfonyl)-4-fluoro-6-(3-fluoroazetidin-1-yl)-1-benzofuran-2-carboxamide,
N-(2-Chloroquinoline-8-sulfonyl)-4-fluoro-6-(3-fluoroazetidin-1-yl)-1-benzofuran-2-carboxamide,
4-Fluoro-6-(3-fluoroazetidin-1-yl)-N-{2-[(propan-2-yl)oxy]benzene-1-sulfonyl}-1-benzofuran-2-carboxamide,
6-(Azetidin-1-yl)-4-fluoro-N-(2-methoxy-4-methylbenzene-1-sulfonyl)-1-benzofuran-2-carboxamide,
4-Fluoro-6-(3-fluoroazetidin-1-yl)-N-{5-methyl-2-[(propan-2-yl)oxy]benzene-1-sulfonyl}-1-benzofuran-2-carboxamide,
4-Fluoro-6-(3-fluoroazetidin-1-yl)-N-(2-methoxy-4-methylbenzene-1-sulfonyl)-1-benzofuran-2-carboxamide,
4-Fluoro-6-(3-fluoroazetidin-1-yl)-N-(2,2,3,3-tetrafluoro-2,3-dihydro-1,4-benzodioxine-5-sulfonyl)-1-benzofuran-2-carboxamide,
6-(azetidin-1-yl)-N-{5-bromo-2-[(propan-2-yl)oxy]benzene-1-sulfonyl}-4-fluoro-1-benzofuran-2-carboxamide,
6-(Azetidin-1-yl)-N-(2-ethylquinoline-8-sulfonyl)-4-fluoro-1-benzofuran-2-carboxamide,
6-(Azetidin-1-yl)-N-(2,3-dihydro-1,4-benzodioxine-5-sulfonyl)-4-fluoro-1-benzofuran-2-carboxamide,
6-(Azetidin-1-yl)-4-fluoro-N-(7-methylquinoline-8-sulfonyl)-1-benzofuran-2-carboxamide
6-(Azetidin-1-yl)-4-fluoro-N-[5-(morpholin-4-yl)naphthalene-1-sulfonyl]-1-benzofuran-2-carboxamide,
6-(Azetidin-1-yl)-N-(3,3-dimethyl-2,3-dihydro-1-benzofuran-5-sulfonyl)-4-fluoro-1-benzofuran-2-carboxamide,
6-(Azetidin-1-yl)-N-[5-(3,5-dimethyl-1,2-oxazol-4-yl)-2-methoxybenzene-1-sulfonyl]-4-fluoro-1-benzofuran-2-carboxamide,
6-(Azetidin-1-yl)-N-[5-(1,3-dimethyl-1H-pyrazol-4-yl)-2-methoxybenzene-1-sulfonyl]-4-fluoro-1-benzofuran-2-carboxamide,
6-(azetidin-1-yl)-N-[5-(3,6-dihydro-2H-pyran-4-yl)-2-methoxybenzene-1-sulfonyl]-4-fluoro-1-benzofuran-2-carboxamide,
6-(Azetidin-1-yl)-4-fluoro-N-(3′-fluoro-4-methoxy[1,1′-biphenyl]-3-sulfonyl)-1-benzofuran-2-carboxamide,
6-(Azetidin-1-yl)-4-fluoro-N-[2-methoxy-5-(1-methyl-1H-pyrazol-4-yl)benzene-1-sulfonyl]-1-benzofuran-2-carboxamide,
6-(Azetidin-1-yl)-4-fluoro-N-[2-methoxy-5-(2-phenylpyrrolidin-1-yl)benzene-1-sulfonyl]-1-benzofuran-2-carboxamide (rac),
6-(Azetidin-1-yl)-4-fluoro-N-[2-methoxy-5-(1-methyl-1,2,3,6-tetrahydropyridin-4-yl)benzene-1-sulfonyl]-1-benzofuran-2-carboxamide,
6-(Azetidin-1-yl)-4-fluoro-N-(2,2,5,6-tetramethyl-2,3-dihydro-1-benzofuran-7-sulfonyl)-1-benzofuran-2-carboxamide,
6-(Azetidin-1-yl)-4-fluoro-N-[2-methoxy-5-(prop-1-en-2-yl)benzene-1-sulfonyl]-1-benzofuran-2-carboxamide,
6-(azetidin-1-yl)-N-(2-ethoxy-3-fluoro-phenyl)sulfonyl-4-fluoro-benzofuran-2-carboxamide,
6-(Azetidin-1-yl)-N-[(5-sec-butyl-2-ethoxyphenyl)sulfonyl]-4-fluoro-1-benzofuran-2-carboxamide (rac),
6-(azetidin-1-yl)-N-[2-ethoxy-5-(trifluoromethyl)phenyl]sulfonyl-4-fluoro-benzofuran-2-carboxamide,
6-(Azetidin-1-yl)-N-(2-ethoxy-5-fluoro-phenyl)sulfonyl-4-fluoro-benzofuran-2-carboxamide,
6-(Azetidin-1-yl)-N-(2-ethoxy-5-phenoxy-phenyl)sulfonyl-4-fluoro-benzofuran-2-carboxamide,
6-(Azetidin-1-yl)-N-[2-ethoxy-5-(trifluoromethoxy)phenyl]sulfonyl-4-fluoro-benzofuran-2-carboxamide,
6-(azetidin-1-yl)-N-(2-benzyloxy-5-isopropyl-phenyl)sulfonyl-4-fluoro-benzofuran-2-carboxamide,
6-(azetidin-1-yl)-N-[2-(cyclopropylmethoxy)-5-isopropyl-phenyl]sulfonyl-4-fluoro-benzofuran-2-carboxamide,
6-(azetidin-1-yl)-4-fluoro-N-[5-isopropyl-2-(2,2,2-trifluoroethoxy)phenyl]sulfonyl-benzofuran-2-carboxamide,
6-(Azetidin-1-yl)-N-[(2-(benzyloxy)-5-(tert-butyl)phenyl]sulfonyl)-4-fluorobenzofuran-2-carboxamide,
6-(Azetidin-1-yl)-N-[(5-(tert-butyl)-2-(cyclopropylmethoxy)phenyl)sulfonyl]-4-fluorobenzofuran-2-carboxamide,
6-(Azetidin-1-yl)-N-[(5-(tert-butyl)-2-(2,2,2-trifluoroethoxy)phenyl)sulfonyl]-4-fluorobenzofuran-2-carboxamide,
6-(Azetidin-1-yl)-N-[(5-(tert-butyl)-2-cyclobutoxyphenyl)sulfonyl]-4-fluorobenzofuran-2-carboxamide,
6-(Azetidin-1-yl)-N-[(5-(tert-butyl)-2-isopropoxyphenyl)sulfonyl]-4-fluorobenzofuran-2-carboxamide,
6-(Azetidin-1-yl)-N-[(2-cyclobutoxy-5-isopropylphenyl)sulfonyl]-4-fluorobenzofuran-2-carboxamide and
6-(Azetidin-1-yl)-N-((2-((2,2-difluorocyclopropyl)methoxy)-5-isopropylphenyl)sulfonyl)-4-fluorobenzofuran-2-carboxamide (rac) In some embodiments, the present invention includes compounds of general formula (I) selected from:
6-(Azetidin-1-yl)-N-[2-(dimethylamino)benzene-1-sulfonyl]-4-fluoro-1-benzofuran-2-carboxamide,
6-(Azetidin-1-yl)-N-(3,4-dihydro-2H-1-benzopyran-8-sulfonyl)-4-fluoro-1-benzofuran-2-carboxamide,
6-(Azetidin-1-yl)-4-fluoro-N-(2-iodobenzene-1-sulfonyl)-1-benzofuran-2-carboxamide,
6-(Azetidin-1-yl)-N-(2-cyclobutyl-6-fluorobenzene-1-sulfonyl)-4-fluoro-1-benzofuran-2-carboxamide,
6-(Azetidin-1-yl)-4-fluoro-N-{2-[(propan-2-yl)oxy]-6-(trifluoromethyl)benzene-1-sulfonyl}-1-benzofuran-2-carboxamide,
6-(Azetidin-1-yl)-N-(5-ethyl-2-methylbenzene-1-sulfonyl)-4-fluoro-1-benzofuran-2-carboxamide,
6-(Azetidin-1-yl)-N-[2-(difluoromethoxy)-4-methylbenzene-1-sulfonyl]-4-fluoro-1-benzofuran-2-carboxamide,
6-(Azetidin-1-yl)-N-(4-ethyl-2-methylthiophene-3-sulfonyl)-4-fluoro-1-benzofuran-2-carboxamide,
6-(Azetidin-1-yl)-N-[4-bromo-2-(propan-2-yl)benzene-1-sulfonyl]-4-fluoro-1-benzofuran-2-carboxamide,
6-(Azetidin-1-yl)-4-fluoro-N-(3-propylbenzene-1-sulfonyl)-1-benzofuran-2-carboxamide,
6-(Azetidin-1-yl)-4-fluoro-N-(1-methyl-1H-indole-7-sulfonyl)-1-benzofuran-2-carboxamide,
6-(Azetidin-1-yl)-4-fluoro-N-{2-fluoro-6-[(propan-2-yl)amino]benzene-1-sulfonyl}-1-benzofuran-2-carboxamide,
6-(Azetidin-1-yl)-4-fluoro-N-(2-fluoro-5-methylbenzene-1-sulfonyl)-1-benzofuran-2-carboxamide,
6-(Azetidin-1-yl)-N-{5-chloro-2-[(propan-2-yl)amino]benzene-1-sulfonyl}-4-fluoro-1-benzofuran-2-carboxamide,
6-(Azetidin-1-yl)-N-[2-(cyclopropylamino)-6-fluorobenzene-1-sulfonyl]-4-fluoro-1-benzofuran-2-carboxamide,
6-(Azetidin-1-yl)-N-[2-(dimethylcarbamoyl)benzene-1-sulfonyl]-4-fluoro-1-benzofuran-2-carboxamide,
6-(Azetidin-1-yl)-4-fluoro-N-[2-propyl-6-(trifluoromethyl)benzene-1-sulfonyl]-1-benzofuran-2-carboxamide,
6-(Azetidin-1-yl)-N-[2-chloro-5-(2-methoxyethoxy)benzene-1-sulfonyl]-4-fluoro-1-benzofuran-2-carboxamide,
6-(Azetidin-1-yl)-N-(2-chloro-5-methylpyridine-3-sulfonyl)-4-fluoro-1-benzofuran-2-carboxamide,
6-(Azetidin-1-yl)-N-(2-cyclopentyl-6-methylbenzene-1-sulfonyl)-4-fluoro-1-benzofuran-2-carboxamide,
2-{[6-(Azetidin-1-yl)-4-fluoro-1-benzofuran-2-carbonyl]sulfamoyl}phenyl
methyl(phenyl)carbamate,
6-(Azetidin-1-yl)-4-fluoro-N-[5-(hydroxymethyl)-2-(trifluoromethoxy)benzene-1-sulfonyl]-1-benzofuran-2-carboxamide,
6-(Azetidin-1-yl)-4-fluoro-N-(2-hydroxy-6-methylbenzene-1-sulfonyl)-1-benzofuran-2-carboxamide,
6-(Azetidin-1-yl)-4-fluoro-N-(5-methoxynaphthalene-1-sulfonyl)-1-benzofuran-2-carboxamide,
6-(Azetidin-1-yl)-N-[5-(dimethylamino)naphthalene-1-sulfonyl]-4-fluoro-1-benzofuran-2-carboxamide,
6-(Azetidin-1-yl)-N-{2-ethyl-6-[(propan-2-yl)oxy]benzene-1-sulfonyl}-4-fluoro-1-benzofuran-2-carboxamide,
6-(Azetidin-1-yl)-N-(2,6-difluoro-3-methylbenzene-1-sulfonyl)-4-fluoro-1-benzofuran-2-carboxamide,
6-(Azetidin-1-yl)-4-fluoro-N-[3-methyl-1-(propan-2-yl)-1H-pyrazole-4-sulfonyl]-1-benzofuran-2-carboxamide,
6-(Azetidin-1-yl)-N-[2-(5,6-dihydro-1,4,2-dioxazin-3-yl)benzene-1-sulfonyl]-4-fluoro-1-benzofuran-2-carboxamide,
6-(Azetidin-1-yl)-N-[2-chloro-5-(dimethylamino)benzene-1-sulfonyl]-4-fluoro-1-benzofuran-2-carboxamide,
N-(5-Acetyl-2-chlorobenzene-1-sulfonyl)-6-(azetidin-1-yl)-4-fluoro-1-benzofuran-2-carboxamide,
Methyl 3-(2-{[6-(azetidin-1-yl)-4-fluoro-1-benzofuran-2-carbonyl]sulfamoyl}phenyl)propanoate,
4-Fluoro-6-(3-fluoroazetidin-1-yl)-N-(5,6,7,8-tetrahydronaphthalene-1-sulfonyl)-1-benzofuran-2-carboxamide,
N-(2,3-dihydro-1,4-benzodioxine-5-sulfonyl)-4-fluoro-6-(3-fluoroazetidin-1-yl)-1-benzofuran-2-carboxamide,
4-Fluoro-6-(3-fluoroazetidin-1-yl)-N-(1,2,3,4-tetrahydroquinoline-8-sulfonyl)-1-benzofuran-2-carboxamide,
N-([1,1′-biphenyl]-2-sulfonyl)-6-(3,3-difluoroazetidin-1-yl)-1-benzofuran-2-carboxamide,
6-(Azetidin-1-yl)-4-fluoro-N-(2,2,3,3-tetrafluoro-2,3-dihydro-1,4-benzodioxine-5-sulfonyl)-1-benzofuran-2-carboxamide,
6-(Azetidin-1-yl)-N-(1,1-dioxo-1,2,3,4-tetrahydro-1-benzothiopyran-8-sulfonyl)-4-fluoro-1-benzofuran-2-carboxamide,
6-(Azetidin-1-yl)-N-(1-ethyl-1H-pyrazolo[3,4-b]pyridine-3-sulfonyl)-4-fluoro-1-benzofuran-2-carboxamide,
6-(Azetidin-1-yl)-4-fluoro-N-[1-methyl-6-(trifluoromethyl)-1H-pyrazolo[3,4-b]pyridine-3-sulfonyl]-1-benzofuran-2-carboxamide,
6-(Azetidin-1-yl)-N-(1,6-dimethyl-1H-pyrazolo[3,4-b]pyridine-3-sulfonyl)-4-fluoro-1-benzofuran-2-carboxamide,
6-[2-(Difluoromethyl)azetidin-1-yl]-4-fluoro-N-(2-methylquinoline-8-sulfonyl)-1-benzofuran-2-carboxamide (rac),
N-(2-ethoxybenzene-1-sulfonyl)-4-fluoro-6-[2-(2-hydroxypropan-2-yl)azetidin-1-yl]-1-benzofuran-2-carboxamide (rac),
6-[2-(Difluoromethyl)azetidin-1-yl]-N-(2-ethoxybenzene-1-sulfonyl)-4-fluoro-1-benzofuran-2-carboxamide (rac),
N-{2,4-difluoro-6-[methyl(propan-2-yl)amino]benzene-1-sulfonyl}-4-fluoro-6-(3-fluoroazetidin-1-yl)-1-benzofuran-2-carboxamide,
6-(Azetidin-1-yl)-N-(4-bromo-2-methoxybenzene-1-sulfonyl)-4-fluoro-1-benzofuran-2-carboxamide,
6-(Azetidin-1-yl)-4-fluoro-N-(6-methylquinoline-8-sulfonyl)-1-benzofuran-2-carboxamide,
6-(Azetidin-1-yl)-4-fluoro-N-(1H-indole-3-sulfonyl)-1-benzofuran-2-carboxamide,
6-(Azetidin-1-yl)-N-(5-bromo-2,3-dihydro-1-benzofuran-7-sulfonyl)-4-fluoro-1-benzofuran-2-carboxamide,
6-(Azetidin-1-yl)-N-(1-benzothiophene-3-sulfonyl)-4-fluoro-1-benzofuran-2-carboxamide,
6-(Azetidin-1-yl)-4-fluoro-N-(2-methyl-2H-benzotriazole-4-sulfonyl)-1-benzofuran-2-carboxamide,
6-(Azetidin-1-yl)-N-(2,1,3-benzothiadiazole-4-sulfonyl)-4-fluoro-1-benzofuran-2-carboxamide,
6-(Azetidin-1-yl)-4-fluoro-N-(5-methoxy-1,2,3-benzothiadiazole-4-sulfonyl)-1-benzofuran-2-carboxamide,
6-(Azetidin-1-yl)-N-(5-tert-butyl-2,3-dimethylbenzene-1-sulfonyl)-4-fluoro-1-benzofuran-2-carboxamide,
tert-butyl 4-(3-{[6-(azetidin-1-yl)-4-fluoro-1-benzofuran-2-carbonyl]sulfamoyl}-4-methoxyphenyl)-3,6-dihydropyridine-1(2H)-carboxylate,
6-(Azetidin-1-yl)-4-fluoro-N-[2-methoxy-5-(pyridin-3-yl)benzene-1-sulfonyl]-1-benzofuran-2-carboxamide,
6-(Azetidin-1-yl)-N-(2-ethoxy-4-fluoro-phenyl)sulfonyl-4-fluoro-benzofuran-2-carboxamide,
6-(2,2-Dimethylazetidin-1-yl)-4-fluoro-N-[(2-methyl-8-quinolyl)sulfonyl]benzofuran-2-carboxamide,
6-(2,2-Dimethylazetidin-1-yl)-N-(2-ethoxyphenyl)sulfonyl-4-fluoro-benzofuran-2-carboxamide,
N-(Benzenesulfonyl)-4-fluoro-6-(2-methylazetidin-1-yl)benzofuran-2-carboxamide (rac),
4-Fluoro-6-(2-methylazetidin-1-yl)-N-[(2-methyl-8-quinolyl)sulfonyl]benzofuran-2-carboxamide (rac),
4-Fluoro-6-(2-methylazetidin-1-yl)-N-(2-phenylphenyl)sulfonyl-benzofuran-2-carboxamide (rac) and
N-(2-Ethoxyphenyl)sulfonyl-4-fluoro-6-(2-methylazetidin-1-yl)benzofuran-2-carboxamide (rac) In some embodiments, the present invention includes compounds of general formula (I) selected from:
6-(Azetidin-1-yl)-N-[2-(3-ethyl-1,2,4-oxadiazol-5-yl)benzene-1-sulfonyl]-4-fluoro-1-benzofuran-2-carboxamide,
6-(Azetidin-1-yl)-N-[2-(benzenesulfonyl)benzene-1-sulfonyl]-4-fluoro-1-benzofuran-2-carboxamide,
6-(azetidin-1-yl)-N-[2-(ethylamino)benzene-1-sulfonyl]-4-fluoro-1-benzofuran-2-carboxamide,
6-(Azetidin-1-yl)-N-{2-[ethyl(methyl)carbamoyl]benzene-1-sulfonyl}-4-fluoro-1-benzofuran-2-carboxamide,
6-(Azetidin-1-yl)-4-fluoro-N-[3-(methoxymethyl)benzene-1-sulfonyl]-1-benzofuran-2-carboxamide,
6-(Azetidin-1-yl)-4-fluoro-N-(3-methoxypyridine-2-sulfonyl)-1-benzofuran-2-carboxamide,
6-(Azetidin-1-yl)-N-[4-(2-cyanoethyl)benzene-1-sulfonyl]-4-fluoro-1-benzofuran-2-carboxamide,
6-(Azetidin-1-yl)-N-(3-bromopyridine-2-sulfonyl)-4-fluoro-1-benzofuran-2-carboxamide,
6-(Azetidin-1-yl)-4-fluoro-N-{2-[(3-methyl-1-phenyl-1H-pyrazol-5-yl)amino]benzene-1-sulfonyl}-1-benzofuran-2-carboxamide,
6-(Azetidin-1-yl)-4-fluoro-N-(3-hydroxynaphthalene-1-sulfonyl)-1-benzofuran-2-carboxamide,
6-(Azetidin-1-yl)-N-[2-chloro-5-(dimethylsulfamoyl)benzene-1-sulfonyl]-4-fluoro-1-benzofuran-2-carboxamide,
6-(Azetidin-1-yl)-4-fluoro-N-(5-propanamidonaphthalene-1-sulfonyl)-1-benzofuran-2-carboxamide
Ethyl 8-{[6-(azetidin-1-yl)-4-fluoro-1-benzofuran-2-carbonyl]sulfamoyl}imidazo[1,2-a]pyridine-2-carboxylate,
6-(Azetidin-1-yl)-N-{2-[(2,2-difluoroethyl)amino]-5-(trifluoromethyl)benzene-1-sulfonyl}-4-fluoro-1-benzofuran-2-carboxamide,
[3-sulfamoyl-4-(trifluoromethoxy)phenyl]methyl 6-(azetidin-1-yl)-4-fluoro-1-benzofuran-2-carboxylate,
6-(Azetidin-1-yl)-4-fluoro-N-(2-methylpyridine-3-sulfonyl)-1-benzofuran-2-carboxamide,
2-{[6-(Azetidin-1-yl)-4-fluoro-1-benzofuran-2-carbonyl]sulfamoyl}-N, N-dimethylpyridine-3-carboxamide,
4-Sulfamoylnaphthalen-2-yl 6-(azetidin-1-yl)-4-fluoro-1-benzofuran-2-carboxylate,
6-(Azetidin-1-yl)-N-[2-chloro-5-(1-hydroxyethyl)benzene-1-sulfonyl]-4-fluoro-1-benzofuran-2-carboxamide (rac),
6-(Azetidin-1-yl)-N-[2-chloro-5-(trifluoromethyl)pyridine-3-sulfonyl]-4-fluoro-1-benzofuran-2-carboxamide,
N-(2-amino-5-chloro-4,6-dimethylpyridine-3-sulfonyl)-6-(azetidin-1-yl)-4-fluoro-1-benzofuran-2-carboxamide,
6-(Azetidin-1-yl)-4-fluoro-N-(4-hydroxynaphthalene-1-sulfonyl)-1-benzofuran-2-carboxamide,
4-Fluoro-6-(3-fluoroazetidin-1-yl)-N-(quinoline-4-sulfonyl)-1-benzofuran-2-carboxamide,
N-[2-(dichloromethyl)benzene-1-sulfonyl]-4-fluoro-6-(3-fluoroazetidin-1-yl)-1-benzofuran-2-carboxamide,
4-Fluoro-6-(3-fluoroazetidin-1-yl)-N-[4-(2-methyl-1,3-thiazol-4-yl)benzene-1-sulfonyl]-1-benzofuran-2-carboxamide,
N-(2-Ethoxybenzene-1-sulfonyl)-6-(3-fluoro-3-methylazetidin-1-yl)-1-benzofuran-2-carboxamide,
N-(2-Ethoxybenzene-1-sulfonyl)-6-[3-(trifluoromethyl)azetidin-1-yl]-1-benzofuran-2-carboxamide,
6-(3,3-Difluoroazetidin-1-yl)-N-(2-ethoxybenzene-1-sulfonyl)-1-benzofuran-2-carboxamide,
N-(2-ethoxybenzene-1-sulfonyl)-6-(3-phenylazetidin-1-yl)-1-benzofuran-2-carboxamide,
6-(3-Ethoxyazetidin-1-yl)-N-(2-ethoxybenzene-1-sulfonyl)-1-benzofuran-2-carboxamide,
6-(5-Azaspiro[2.3]hexan-5-yl)-N-(2-ethoxybenzene-1-sulfonyl)-1-benzofuran-2-carboxamide,
6-(2-Azaspiro[3.3]heptan-2-yl)-N-(2-ethoxybenzene-1-sulfonyl)-1-benzofuran-2-carboxamide,
6-(3,3-dimethylazetidin-1-yl)-N-(2-ethoxybenzene-1-sulfonyl)-1-benzofuran-2-carboxamide,
N-(2-ethoxybenzene-1-sulfonyl)-6-(2-oxa-6-azaspiro[3.3]heptan-6-yl)-1-benzofuran-2-carboxamide,
6-(Azetidin-1-yl)-N-(5-chloro-2,2-difluoro-2H-1,3-benzodioxole-4-sulfonyl)-4-fluoro-1-benzofuran-2-carboxamide,
6-(Azetidin-1-yl)-4-fluoro-N-[2-(methylsulfanyl)imidazo[1,2-a]pyridine-3-sulfonyl]-1-benzofuran-2-carboxamide,
6-(Azetidin-1-yl)-N-[2-(ethanesulfonyl)imidazo[1,2-a]pyridine-3-sulfonyl]-4-fluoro-1-benzofuran-2-carboxamide,
6-(Azetidin-1-yl)-N-(6,8-dichloroimidazo[1,2-a]pyridine-3-sulfonyl)-4-fluoro-1-benzofuran-2-carboxamide,
6-(Azetidin-1-yl)-4-fluoro-N-(isoquinoline-5-sulfonyl)-1-benzofuran-2-carboxamide,
Methyl (5-{[6-(azetidin-1-yl)-4-fluoro-1-benzofuran-2-carbonyl]sulfamoyl}naphthalen-1-yl)carbamate,
6-(Azetidin-1-yl)-4-fluoro-N-(4-methylisoquinoline-5-sulfonyl)-1-benzofuran-2-carboxamide,
6-(Azetidin-1-yl)-4-fluoro-N-[2-methoxy-5-(4-methylpiperazin-1-yl)benzene-1-sulfonyl]-1-benzofuran-2-carboxamide,
N-(benzenesulfonyl)-6-(2,2-dimethylazetidin-1-yl)-4-fluoro-benzofuran-2-carboxamide and
6-(2,2-Dimethylazetidin-1-yl)-4-fluoro-N-(2-phenylphenyl)sulfonyl-benzofuran-2-carboxamide.
In some embodiments, the present invention includes compounds of general formula (I) selected from:
6-(Azetidin-1-yl)-N-(2-ethoxy-6-methoxybenzene-1-sulfonyl)-4-fluoro-1-benzofuran-2-carboxamide,
6-(Azetidin-1-yl)-N-(2-ethoxybenzene-1-sulfonyl)-4-fluoro-1-benzofuran-2-carboxamide,
6-(Azetidin-1-yl)-N-(2-{[butan-2-yl]oxy}-5-methylbenzene-1-sulfonyl)-4-fluoro-1-benzofuran-2-carboxamide,
6-(Azetidin-1-yl)-N-[2-(benzyloxy)-6-(cyclobutyloxy)benzene-1-sulfonyl]-4-fluoro-1-benzofuran-2-carboxamide,
6-(Azetidin-1-yl)-N-[2-(benzyloxy)-6-ethoxybenzene-1-sulfonyl]-4-fluoro-1-benzofuran-2-carboxamide,
6-(Azetidin-1-yl)-N-[2-(cyclopentyloxy)-5-methylbenzene-1-sulfonyl]-4-fluoro-1-benzofuran-2-carboxamide,
6-(Azetidin-1-yl)-N-(2-ethoxy-6-propoxybenzene-1-sulfonyl)-4-fluoro-1-benzofuran-2-carboxamide,
6-(Azetidin-1-yl)-4-fluoro-N-(2-{[-1,1,1-trifluoropropan-2-yl]oxy}benzene-1-sulfonyl)-1-benzofuran-2-carboxamide (rac),
6-(Azetidin-1-yl)-4-fluoro-N-(2-{[-1,1,1-trifluoropropan-2-yl]oxy}benzene-1-sulfonyl)-1-benzofuran-2-carboxamide (Enantiomer 1),
6-(Azetidin-1-yl)-4-fluoro-N-(2-{[-1,1,1-trifluoropropan-2-yl]oxy}benzene-1-sulfonyl)-1-benzofuran-2-carboxamide (Enantiomer 2),
6-(Azetidin-1-yl)-N-[2-(cyclobutyloxy)-6-fluorobenzene-1-sulfonyl]-4-fluoro-1-benzofuran-2-carboxamide,
6-(Azetidin-1-yl)-N-(2,6-dipropoxybenzene-1-sulfonyl)-4-fluoro-1-benzofuran-2-carboxamide,
6-(Azetidin-1-yl)-4-fluoro-N-[2-(3-methyl-1,2,4-oxadiazol-5-yl)benzene-1-sulfonyl]-1-benzofuran-2-carboxamide,
6-(azetidin-1-yl)-N-(5-tert-butyl-2-ethoxybenzene-1-sulfonyl)-4-fluoro-1-benzofuran-2-carboxamide,
6-(Azetidin-1-yl)-N-[5-bromo-2-(trifluoromethoxy)benzene-1-sulfonyl]-4-fluoro-1-benzofuran-2-carboxamide,
N-(2-Ethoxybenzene-1-sulfonyl)-6-(3-fluoroazetidin-1-yl)-1-benzofuran-2-carboxamide,
N-(5-tert-butyl-2-ethoxybenzene-1-sulfonyl)-4-fluoro-6-(3-fluoroazetidin-1-yl)-1-benzofuran-2-carboxamide,
Sodium [6-(azetidin-1-yl)-4-fluoro-1-benzofuran-2-carbonyl](2-ethoxybenzene-1-sulfonyl)azanide,
6-(Azetidin-1-yl)-N-(5-tert-butyl-2-methoxybenzene-1-sulfonyl)-4-fluoro-1-benzofuran-2-carboxamide,
6-(Azetidin-1-yl)-N-[2-ethoxy-5-(propan-2-yl)benzene-1-sulfonyl]-4-fluoro-1-benzofuran-2-carboxamide,
6-(Azetidin-1-yl)-4-fluoro-N-{2-[(propan-2-yl)oxy]benzene-1-sulfonyl}-1-benzofuran-2-carboxamide,
6-(Azetidin-1-yl)-N-[2-(cyclopropyloxy)benzene-1-sulfonyl]-4-fluoro-1-benzofuran-2-carboxamide,
6-(Azetidin-1-yl)-N-(2-chloroquinoline-8-sulfonyl)-4-fluoro-1-benzofuran-2-carboxamide,
6-(Azetidin-1-yl)-4-fluoro-N-(2-propoxybenzene-1-sulfonyl)-1-benzofuran-2-carboxamide,
6-(Azetidin-1-yl)-N-(5-bromo-2-ethoxybenzene-1-sulfonyl)-4-fluoro-1-benzofuran-2-carboxamide,
6-(azetidin-1-yl)-N-(2-ethyl-6-methoxybenzene-1-sulfonyl)-4-fluoro-1-benzofuran-2-carboxamide,
6-(Azetidin-1-yl)-N-(5-bromo-2-methoxybenzene-1-sulfonyl)-4-fluoro-1-benzofuran-2-carboxamide,
N-(5-tert-butyl-2-methoxybenzene-1-sulfonyl)-4-fluoro-6-(3-fluoroazetidin-1-yl)-1-benzofuran-2-carboxamide,
N-[2-ethoxy-5-(propan-2-yl)benzene-1-sulfonyl]-4-fluoro-6-(3-fluoroazetidin-1-yl)-1-benzofuran-2-carboxamide,
N-(2-ethoxy-5-ethylbenzene-1-sulfonyl)-4-fluoro-6-(3-fluoroazetidin-1-yl)-1-benzofuran-2-carboxamide,
4-Fluoro-6-(3-fluoroazetidin-1-yl)-N-{2-[(propan-2-yl)oxy]benzene-1-sulfonyl}-1-benzofuran-2-carboxamide,
6-(Azetidin-1-yl)-4-fluoro-N-(2-methoxy-4-methylbenzene-1-sulfonyl)-1-benzofuran-2-carboxamide,
6-(Azetidin-1-yl)-N-[5-(3,5-dimethyl-1,2-oxazol-4-yl)-2-methoxybenzene-1-sulfonyl]-4-fluoro-1-benzofuran-2-carboxamide,
6-(Azetidin-1-yl)-N-[5-(1,3-dimethyl-1H-pyrazol-4-yl)-2-methoxybenzene-1-sulfonyl]-4-fluoro-1-benzofuran-2-carboxamide,
6-(Azetidin-1-yl)-4-fluoro-N-[2-methoxy-5-(1-methyl-1H-pyrazol-4-yl)benzene-1-sulfonyl]-1-benzofuran-2-carboxamide,
6-(Azetidin-1-yl)-4-fluoro-N-[2-methoxy-5-(2-phenylpyrrolidin-1-yl)benzene-1-sulfonyl]-1-benzofuran-2-carboxamide (rac),
6-(Azetidin-1-yl)-N-(2-ethoxy-5-fluoro-phenyl)sulfonyl-4-fluoro-benzofuran-2-carboxamide,
6-(Azetidin-1-yl)-N-(2-ethoxy-5-phenoxy-phenyl)sulfonyl-4-fluoro-benzofuran-2-carboxamide,
6-(Azetidin-1-yl)-N-[2-ethoxy-5-(trifluoromethoxy)phenyl]sulfonyl-4-fluoro-benzofuran-2-carboxamide,
6-(azetidin-1-yl)-N-[2-(cyclopropylmethoxy)-5-isopropyl-phenyl]sulfonyl-4-fluoro-benzofuran-2-carboxamide,
6-(azetidin-1-yl)-4-fluoro-N-[5-isopropyl-2-(2,2,2-trifluoroethoxy)phenyl]sulfonyl-benzofuran-2-carboxamide and
6-(Azetidin-1-yl)-N-[(5-(tert-butyl)-2-(cyclopropylmethoxy)phenyl)sulfonyl]-4-fluorobenzofuran-2-carboxamide.
In some embodiments, the present invention includes compounds of general formula (I) selected from:
6-(Azetidin-1-yl)-N-[2-(cyclopentyloxy)-5-ethylbenzene-1-sulfonyl]-4-fluoro-1-benzofuran-2-carboxamide,
6-(Azetidin-1-yl)-4-fluoro-N-(7-methoxyquinoline-8-sulfonyl)-1-benzofuran-2-carboxamide,
6-(Azetidin-1-yl)-N-[2-(dimethylamino)benzene-1-sulfonyl]-4-fluoro-1-benzofuran-2-carboxamide,
6-(Azetidin-1-yl)-N-([1,1′-biphenyl]-2-sulfonyl)-4-fluoro-1-benzofuran-2-carboxamide,
Sodium {[6-(azetidin-1-yl)-4-fluoro-1-benzofuran-2-yl]carbonyl}([biphenyl]-2-ylsulfonyl)azanide,
6-(Azetidin-1-yl)-N-(2,2-dimethyl-2,3-dihydro-1-benzofuran-7-sulfonyl)-4-fluoro-1-benzofuran-2-carboxamide,
6-(Azetidin-1-yl)-N-[2-ethoxy-6-(propan-2-yl)benzene-1-sulfonyl]-4-fluoro-1-benzofuran-2-carboxamide,
6-(Azetidin-1-yl)-N-(2-ethoxy-6-methylbenzene-1-sulfonyl)-4-fluoro-1-benzofuran-2-carboxamide,
6-(Azetidin-1-yl)-N-([1,1′-biphenyl]-2-sulfonyl)-1-benzofuran-2-carboxamide,
6-(Azetidin-1-yl)-N-(3-chloro-6-methoxy-2-methylbenzene-1-sulfonyl)-4-fluoro-1-benzofuran-2-carboxamide,
6-(Azetidin-1-yl)-N-{2-chloro-6-[(propan-2-yl)oxy]benzene-1-sulfonyl}-4-fluoro-1-benzofuran-2-carboxamide,
6-(Azetidin-1-yl)-N-[2,5-di(propan-2-yl)benzene-1-sulfonyl]-4-fluoro-1-benzofuran-2-carboxamide,
6-(Azetidin-1-yl)-4-fluoro-N-(2-propoxy-6-propylbenzene-1-sulfonyl)-1-benzofuran-2-carboxamide,
6-(Azetidin-1-yl)-4-fluoro-N-[2-methyl-5-(propan-2-yl)benzene-1-sulfonyl]-1-benzofuran-2-carboxamide,
6-(Azetidin-1-yl)-N-{2-bromo-6-[(propan-2-yl)oxy]benzene-1-sulfonyl}-4-fluoro-1-benzofuran-2-carboxamide,
6-(Azetidin-1-yl)-4-fluoro-N-[2-methyl-6-(propan-2-yl)benzene-1-sulfonyl]-1-benzofuran-2-carboxamide,
6-(Azetidin-1-yl)-N-{2-ethyl-6-[(propan-2-yl)oxy]benzene-1-sulfonyl}-4-fluoro-1-benzofuran-2-carboxamide,
6-(Azetidin-1-yl)-N-[2-(benzyloxy)-6-ethylbenzene-1-sulfonyl]-4-fluoro-1-benzofuran-2-carboxamide,
6-(Azetidin-1-yl)-4-fluoro-N-{2-methyl-6-[(propan-2-yl)oxy]benzene-1-sulfonyl}-1-benzofuran-2-carboxamide,
6-(Azetidin-1-yl)-4-fluoro-N-(2-methylquinoline-8-sulfonyl)-1-benzofuran-2-carboxamide
6-(Azetidin-1-yl)-4-fluoro-N-[2-(trifluoromethoxy)benzene-1-sulfonyl]-1-benzofuran-2-carboxamide,
4-Fluoro-6-(3-fluoroazetidin-1-yl)-N-(2-methylquinoline-8-sulfonyl)-1-benzofuran-2-carboxamide,
N-(2-Chloroquinoline-8-sulfonyl)-4-fluoro-6-(3-fluoroazetidin-1-yl)-1-benzofuran-2-carboxamide,
4-Fluoro-6-(3-fluoroazetidin-1-yl)-N-(2,2,3,3-tetrafluoro-2,3-dihydro-1,4-benzodioxine-5-sulfonyl)-1-benzofuran-2-carboxamide,
6-(Azetidin-1-yl)-N-(2-ethylquinoline-8-sulfonyl)-4-fluoro-1-benzofuran-2-carboxamide,
6-(Azetidin-1-yl)-4-fluoro-N-(7-methylquinoline-8-sulfonyl)-1-benzofuran-2-carboxamide
6-(Azetidin-1-yl)-N-(3,3-dimethyl-2,3-dihydro-1-benzofuran-5-sulfonyl)-4-fluoro-1-benzofuran-2-carboxamide,
6-(azetidin-1-yl)-N-[5-(3,6-dihydro-2H-pyran-4-yl)-2-methoxybenzene-1-sulfonyl]-4-fluoro-1-benzofuran-2-carboxamide,
tert-butyl 4-(3-{[6-(azetidin-1-yl)-4-fluoro-1-benzofuran-2-carbonyl]sulfamoyl}-4-methoxyphenyl)-3,6-dihydropyridine-1(2H)-carboxylate,
6-(Azetidin-1-yl)-4-fluoro-N-(3′-fluoro-4-methoxy[1,1′-biphenyl]-3-sulfonyl)-1-benzofuran-2-carboxamide,
6-(Azetidin-1-yl)-4-fluoro-N-[2-methoxy-5-(1-methyl-1,2,3,6-tetrahydropyridin-4-yl)benzene-1-sulfonyl]-1-benzofuran-2-carboxamide,
6-(Azetidin-1-yl)-4-fluoro-N-[2-methoxy-5-(prop-1-en-2-yl)benzene-1-sulfonyl]-1-benzofuran-2-carboxamide,
6-(azetidin-1-yl)-N-(2-ethoxy-3-fluoro-phenyl)sulfonyl-4-fluoro-benzofuran-2-carboxamide,
6-(Azetidin-1-yl)-N-[(5-(tert-butyl)-2-(2,2,2-trifluoroethoxy)phenyl)sulfonyl]-4-fluorobenzofuran-2-carboxamide,
6-(Azetidin-1-yl)-N-[(5-(tert-butyl)-2-cyclobutoxyphenyl)sulfonyl]-4-fluorobenzofuran-2-carboxamide and
6-(Azetidin-1-yl)-N-[(5-(tert-butyl)-2-isopropoxyphenyl)sulfonyl]-4-fluorobenzofuran-2-carboxamide
In some embodiments, the present invention includes compounds of general formula (I) selected from:
6-(Azetidin-1-yl)-4-fluoro-N-[2-(3,3,3-trifluoropropyl)benzene-1-sulfonyl]-1-benzofuran-2-carboxamide,
6-(Azetidin-1-yl)-N-[2-chloro-5-(ethoxymethyl)benzene-1-sulfonyl]-4-fluoro-1-benzofuran-2-carboxamide,
6-(Azetidin-1-yl)-4-fluoro-N-{2-[(1E)-3,3,3-trifluoroprop-1-en-1-yl]benzene-1-sulfonyl}-1-benzofuran-2-carboxamide,
6-(Azetidin-1-yl)-4-fluoro-N-(quinoxaline-5-sulfonyl)-1-benzofuran-2-carboxamide,
Methyl (5-{[6-(azetidin-1-yl)-4-fluoro-1-benzofuran-2-carbonyl]sulfamoyl}naphthalen-2-yl)acetate,
6-(Azetidin-1-yl)-4-fluoro-N-[2-propyl-6-(trifluoromethyl)benzene-1-sulfonyl]-1-benzofuran-2-carboxamide,
6-(Azetidin-1-yl)-N-[2-chloro-5-(2-methoxyethoxy)benzene-1-sulfonyl]-4-fluoro-1-benzofuran-2-carboxamide,
6-(Azetidin-1-yl)-4-fluoro-N-(5-methoxynaphthalene-1-sulfonyl)-1-benzofuran-2-carboxamide,
6-(Azetidin-1-yl)-N-[5-(dimethylamino)naphthalene-1-sulfonyl]-4-fluoro-1-benzofuran-2-carboxamide,
6-(Azetidin-1-yl)-4-fluoro-N-[2-(morpholine-4-carbonyl)benzene-1-sulfonyl]-1-benzofuran-2-carboxamide,
Methyl (2E)-3-(2-{[6-(azetidin-1-yl)-4-fluoro-1-benzofuran-2-carbonyl]sulfamoyl}phenyl)prop-2-enoate,
6-(Azetidin-1-yl)-N-(3-ethoxybenzene-1-sulfonyl)-4-fluoro-1-benzofuran-2-carboxamide
2-{[6-(Azetidin-1-yl)-4-fluoro-1-benzofuran-2-carbonyl]sulfamoyl}phenyl diethylcarbamate,
6-(Azetidin-1-yl)-N-[2-(ethanesulfonyl)benzene-1-sulfonyl]-4-fluoro-1-benzofuran-2-carboxamide,
6-(azetidin-1-yl)-4-fluoro-N-(4-methylnaphthalene-1-sulfonyl)-1-benzofuran-2-carboxamide,
6-(Azetidin-1-yl)-4-fluoro-N-[3-methyl-1-(propan-2-yl)-1H-pyrazole-4-sulfonyl]-1-benzofuran-2-carboxamide,
6-(Azetidin-1-yl)-N-[2-(5,6-dihydro-1,4,2-dioxazin-3-yl)benzene-1-sulfonyl]-4-fluoro-1-benzofuran-2-carboxamide,
Propan-2-yl 2-{[6-(azetidin-1-yl)-4-fluoro-1-benzofuran-2-carbonyl]sulfamoyl}benzoate,
Methyl 3-(2-{[6-(azetidin-1-yl)-4-fluoro-1-benzofuran-2-carbonyl]sulfamoyl}phenyl)propanoate,
N-(2,3-dihydro-1,4-benzodioxine-5-sulfonyl)-4-fluoro-6-(3-fluoroazetidin-1-yl)-1-benzofuran-2-carboxamide,
4-Fluoro-6-(3-fluoroazetidin-1-yl)-N-[4-(2-methyl-1,3-thiazol-4-yl)benzene-1-sulfonyl]-1-benzofuran-2-carboxamide,
6-(Azetidin-1-yl)-N-(1,6-dimethyl-1H-pyrazolo[3,4-b]pyridine-3-sulfonyl)-4-fluoro-1-benzofuran-2-carboxamide,
6-(Azetidin-1-yl)-N-(1,4-dimethyl-1H-pyrazolo[3,4-b]pyridine-3-sulfonyl)-4-fluoro-1-benzofuran-2-carboxamide,
6-(Azetidin-1-yl)-N-(2-chloro-6-propylimidazo[1,2-b]pyridazine-3-sulfonyl)-4-fluoro-1-benzofuran-2-carboxamide,
6-(Azetidin-1-yl)-N-[2-(ethylsulfanyl)imidazo[1,2-a]pyridine-3-sulfonyl]-4-fluoro-1-benzofuran-2-carboxamide,
N-{2,6-difluoro-4-[methyl(propan-2-yl)amino]benzene-1-sulfonyl}-4-fluoro-6-(3-fluoroazetidin-1-yl)-1-benzofuran-2-carboxamide,
N-{2,4-difluoro-6-[methyl(propan-2-yl)amino]benzene-1-sulfonyl}-4-fluoro-6-(3-fluoroazetidin-1-yl)-1-benzofuran-2-carboxamide,
6-(Azetidin-1-yl)-4-fluoro-N-(2-methyl-2H-benzotriazole-4-sulfonyl)-1-benzofuran-2-carboxamide,
6-(Azetidin-1-yl)-N-(2,1,3-benzothiadiazole-4-sulfonyl)-4-fluoro-1-benzofuran-2-carboxamide,
6-(Azetidin-1-yl)-4-fluoro-N-(5-methoxy-1,2,3-benzothiadiazole-4-sulfonyl)-1-benzofuran-2-carboxamide,
6-(Azetidin-1-yl)-N-(3,4-dichloro-2-methoxybenzene-1-sulfonyl)-4-fluoro-1-benzofuran-2-carboxamide and
6-(Azetidin-1-yl)-4-fluoro-N-[5-(morpholin-4-yl)naphthalene-1-sulfonyl]-1-benzofuran-2-carboxamide.

### Further Embodiments of the First Aspect of the Present Invention

In some embodiments, the present invention provides compounds of formula (I), supra, in which R¹ is a hydrogen atom or a tautomer, or an N-oxide, or a salt thereof, or a salt of a tautomer, or a salt of an N-oxide, or a mixture of same.

In some embodiments, the present invention provides compounds of formula (I), supra, in which R² is a hydrogen atom or a tautomer, or an N-oxide, or a salt thereof, or a salt of a tautomer, or a salt of an N-oxide, or a mixture of same.

In some embodiments, the present invention provides compounds of formula (I), supra, in which R³ is selected from a hydrogen atom and a halogen atom or a tautomer, or an N-oxide, or a salt thereof, or a salt of a tautomer, or a salt of an N-oxide, or a mixture of same.

In some embodiments, the present invention provides compounds of formula (I), supra, in which R³ is selected from a hydrogen atom and a fluorine atom or a tautomer, or an N-oxide, or a salt thereof, or a salt of a tautomer, or a salt of an N-oxide, or a mixture of same.

In some embodiments, the present invention provides compounds of formula (I), supra, in which R³ is a hydrogen atom or a tautomer, or an N-oxide, or a salt thereof, or a salt of a tautomer, or a salt of an N-oxide, or a mixture of same.

In some embodiments, the present invention provides compounds of formula (I), supra, in which R³ is a halogen atom or a tautomer, or an N-oxide, or a salt thereof, or a salt of a tautomer, or a salt of an N-oxide, or a mixture of same.

In some embodiments, the present invention provides compounds of formula (I), supra, in which R³ is a fluorine atom or a tautomer, or an N-oxide, or a salt thereof, or a salt of a tautomer, or a salt of an N-oxide, or a mixture of same.

In some embodiments, the present invention provides compounds of formula (I), supra, in which R⁴ is a hydrogen atom or a tautomer, or an N-oxide, or a salt thereof, or a salt of a tautomer, or a salt of an N-oxide, or a mixture of same.

In some embodiments, the present invention provides compounds of formula (I), supra, in which R⁵ is a hydrogen atom or a tautomer, or an N-oxide, or a salt thereof, or a salt of a tautomer, or a salt of an N-oxide, or a mixture of same.

In some embodiments, the present invention provides compounds of formula (I), supra, in which R¹, R², R⁴ and R⁵ are each a hydrogen atom or a tautomer, or an N-oxide, or a salt thereof, or a salt of a tautomer, or a salt of an N-oxide, or a mixture of same.

In some embodiments, the present invention provides compounds of formula (I), supra, in which R¹, R², R⁴ and R⁵ are each a hydrogen atom and R³ is selected from a hydrogen atom and a halogen atom or a tautomer, or an N-oxide, or a salt thereof, or a salt of a tautomer, or a salt of an N-oxide, or a mixture of same.

In some embodiments, the present invention provides compounds of formula (I), supra, in which R¹, R², R⁴ and R⁵ are each a hydrogen atom and R³ is selected from a hydrogen atom and a fluorine atom or a tautomer, or an N-oxide, or a salt thereof, or a salt of a tautomer, or a salt of an N-oxide, or a mixture of same.

In some embodiments, the present invention provides compounds of formula (I), supra, in which R¹, R², R⁴ and R⁵ are each a hydrogen atom and R³ is a hydrogen atom or a tautomer, or an N-oxide, or a salt thereof, or a salt of a tautomer, or a salt of an N-oxide, or a mixture of same.

In some embodiments, the present invention provides compounds of formula (I), supra, in which R¹, R², R⁴ and R⁵ are each a hydrogen atom and R³ is a fluorine atom or a tautomer, or an N-oxide, or a salt thereof, or a salt of a tautomer, or a salt of an N-oxide, or a mixture of same.

In some embodiments, the present invention provides compounds of formula (I), supra, in which
R⁶ is selected from a C₁-C₆-alkyl group, a phenyl group, a naphthyl group, a heteroaryl group, a (phenyl)-(C₁-C₆-alkyl)- group, a (naphthyl)-(C₁-C₆-alkyl)- group, a (heteroaryl)-(C₁-C₆-alkyl)- group, a (heterocycloalkyl)-(C₁-C₆-alkyl)- group and a heterocycloalkyl group,
   wherein said heterocycloalkyl group is connected to the rest of the molecule via a carbon atom of said heterocycloalkyl group,
   wherein said phenyl, naphthyl, heteroaryl or heterocycloalkyl group is each optionally substituted with one or more substituents independently selected from a halogen atom, a cyano group, a nitro group, a hydroxy group, a C₁-C₆-alkyl group, a C₁-C₆-hydroxyalkyl group, a C₁-C₆-alkylsulfanyl group, a C₁-C₆-haloalkyl group, a C₁-C₆-alkoxy group, a (C₁-C₃-alkyl)-O—(C₁-C₂-alkyl)- group, a (C₁-C₃-alkyl)-O—(C₁-C₂-alkyl)-O— group, a (C₃-C₃-cycloalkyl)-(C₁-C₂-alkyl)-O— group, a (C₃-C₃-halocycloalkyl)-(C₁-C₂-alkyl)-O— group, a (phenyl)-(C₁-C₂-alkyl)-O— group, a C₁-C₆-haloalkoxy group, a C₂-C₆-alkenyl group, a C₂-C₆-alkynyl group, a C₃-C₃-cycloalkyl group, a C₄-C₃-cycloalkenyl group, a C₃-C₃-cycloalkoxy group, a C₁-C₆-thioalkyl group, a heterocycloalkyl group, a R⁷R⁸N— group, a (C₁-C₂-alkyl)-S(═O)₂— group, a (R⁷R⁸N)—S(═O)₂— group, a (R⁷R⁸N)—(C₁-C₆-alkyl)-(C═O)—NH— group, a (H₃C)—(C═O)— group, a (R⁷R⁸N)—(C═O)— group, a (R⁷R⁸N)—(C═O)—O— group, a (R⁷R⁸N)—(C₁-C₆-alkyl)- group, a R⁹OOC—(C₁-C₃-alkyl)- group, a R⁹OOC— group, a phenyl group, a naphthyl group, a (phenyl)-O— group and a heteroaryl group,
      wherein said heterocycloalkyl group is connected to the rest of the molecule via a carbon atom of said heterocycloalkyl group,
      wherein said phenyl, naphthyl, heteroaryl or heterocycloalkyl group is each optionally substituted with one or more substituents independently selected from a halogen atom, a cyano group, a nitro group, a C₁-C₆-alkyl group, a C₁-C₆-hydroxyalkyl group, a C₁-C₆-alkylsulfanyl group, a C₁-C₆-haloalkyl group, a C₁-C₆-alkoxy group, a C₁-C₆-haloalkoxy group, a C₂-C₆-alkenyl group, a C₂-C₆-alkynyl group, a C₃-C₃-cycloalkyl group, a C₄-C₃-cycloalkenyl group, a C₃-C₃-cycloalkoxy group, a heterocycloalkyl group, a phenyl group, a R⁷R⁸N— group, a (R⁷R⁸N)—S(═O)₂— group, a (R⁷R⁸N)—(C₁-C₆-alkyl)-(C═O)—NH— group, a (R⁷R⁸N)—(C₁-C₆-alkyl)- group, and a a R⁹OOC— group,
         wherein said heterocycloalkyl group is connected to the rest of the molecule via a carbon atom of said heterocycloalkyl group;
or a tautomer, or an N-oxide, or a salt thereof, or a salt of a tautomer, or a salt of an N-oxide, or a mixture of same.

In some embodiments, the present invention provides compounds of formula (I), supra, in which
R⁶ is selected from a phenyl group, a naphthyl group and a heteroaryl group,
   wherein said phenyl, naphthyl or heteroaryl group is each optionally substituted with one or more substituents independently selected from a halogen atom, a hydroxy group, a C₁-C₆-alkyl group, a C₁-C₆-hydroxyalkyl group, a C₁-C₆-haloalkyl group, a C₁-C₆-alkoxy group, a (C₁-C₃-alkyl)-O—(C₁-C₂-alkyl)- group, a (C₁-C₃-alkyl)-O—(C₁-C₂-alkyl)-O— group, a (C₃-C₃-cycloalkyl)-(C₁-C₂-alkyl)-O— group, a (C₃-C₃-halocycloalkyl)-(C₁-C₂-alkyl)-O— group, a (phenyl)-(C₁-C₂-alkyl)-O— group, a C₁-C₆-haloalkoxy group, a C₂-C₆-alkenyl group, a C₃-C₃-cycloalkyl group, a C₃-C₃-cycloalkoxy group, a C₁-C₆-thioalkyl group, a heterocycloalkyl group, a R⁷R⁸N— group, a (C₁-C₂-alkyl)-S(═O)₂— group, a (phenyl)-S(═O)₂— group, a (R⁷R⁸N)—S(═O)₂— group, a (H₃C)—(C═O)— group, a (R⁷R⁸N)—(C═O)— group, a (R⁷R⁸N)—(C═O)—O— group, a R⁹OOC—(C₁-C₃-alkyl)- group, a R⁹OOC— group, a phenyl group, a (phenyl)-O— group and a heteroaryl group,
      wherein said heterocycloalkyl group is connected to the rest of the molecule via a carbon atom of said heterocycloalkyl group,
      wherein said phenyl, heteroaryl or heterocycloalkyl group is each optionally substituted with one or more substituents independently selected from a halogen atom, a C₁-C₃-alkyl group, a phenyl group and a R⁹OOC— group;
or a tautomer, or an N-oxide, or a salt thereof, or a salt of a tautomer, or a salt of an N-oxide, or a mixture of same.

In some embodiments, the present invention provides compounds of formula (I), supra, in which
R⁶ is selected from a phenyl group, a naphthyl group and a heteroaryl group,
   wherein said phenyl, naphthyl or heteroaryl group is each optionally substituted with one or more substituents independently selected from a halogen atom, a hydroxy group, a C₁-C₆-alkyl group, a C₁-C₆-hydroxyalkyl group, a C₁-C₆-haloalkyl group, a C₁-C₆-alkoxy group, a (C₁-C₃-alkyl)-O—(C₁-C₂-alkyl)- group, a (C₁-C₃-alkyl)-O—(C₁-C₂-alkyl)-O— group, a (C₃-C₃-cycloalkyl)-(C₁-C₂-alkyl)-O— group, a (C₃-C₃-halocycloalkyl)-(C₁-C₂-alkyl)-O— group, a (phenyl)-(C₁-C₂-alkyl)-O— group, a C₁-C₆-haloalkoxy group, a C₂-C₆-alkenyl group, a C₃-C₃-cycloalkyl group, a C₃-C₃-cycloalkoxy group, a C₁-C₆-thioalkyl group, a heterocycloalkyl group, a R⁷R⁸N— group, a (C₁-C₂-alkyl)-S(═O)₂— group, a (phenyl)-S(═O)₂— group, a (R⁷R⁸N)—S(═O)₂— group, a (H₃C)—(C═O)— group, a (R⁷R⁸N)—(C═O)— group, a (R⁷R⁸N)—(C═O)—O— group, a R⁹OOC—(C₁-C₃-alkyl)- group, a R⁹OOC— group, a phenyl group, a (phenyl)-O— group and a heteroaryl group,
   and wherein, when substituted, said phenyl group is preferably substituted in one or two of the ortho-positions with respect to the point of attachment of said phenyl group to the rest of the molecule,
      wherein said heterocycloalkyl group is connected to the rest of the molecule via a carbon atom of said heterocycloalkyl group,
      wherein said phenyl, heteroaryl or heterocycloalkyl group is each optionally substituted with one or more substituents independently selected from a halogen atom, a C₁-C₃-alkyl group, a phenyl group and a R⁹OOC— group;
or a tautomer, or an N-oxide, or a salt thereof, or a salt of a tautomer, or a salt of an N-oxide, or a mixture of same.

In some embodiments, the present invention provides compounds of formula (I), supra, in which
R⁶ is selected from a phenyl group, a naphthyl group and a heteroaryl group,
   wherein said phenyl, naphthyl or heteroaryl group is each optionally substituted with one or more substituents independently selected from a halogen atom, a hydroxy group, a C₁-C₆-alkyl group, a C₁-C₆-hydroxyalkyl group, a C₁-C₆-haloalkyl group, a C₁-C₆-alkoxy group, a (C₁-C₃-alkyl)-O—(C₁-C₂-alkyl)- group, a (C₁-C₃-alkyl)-O—(C₁-C₂-alkyl)-O— group, a (C₃-C₃-cycloalkyl)-(C₁-C₂-alkyl)-O— group, a (C₃-C₃-halocycloalkyl)-(C₁-C₂-alkyl)-O— group, a (phenyl)-(C₁-C₂-alkyl)-O— group, a C₁-C₆-haloalkoxy group, a C₂-C₆-alkenyl group, a C₃-C₃-cycloalkyl group, a C₃-C₃-cycloalkoxy group, a C₁-C₆-thioalkyl group, a heterocycloalkyl group, a R⁷R⁸N— group, a (C₁-C₂-alkyl)-S(═O)₂— group, a (phenyl)-S(═O)₂— group, a (R⁷R⁸N)—S(═O)₂— group, a (H₃C)—(C═O)— group, a (R⁷R⁸N)—(C═O)— group, a (R⁷R⁸N)—(C═O)—O— group, a R⁹OOC—(C₁-C₃-alkyl)- group, a R⁹OOC— group, a phenyl group, a (phenyl)-O— group and a heteroaryl group,
   and wherein, when substituted, said phenyl group is preferably substituted with a C₁-C₄-alkoxy group in one or two of the ortho-positions with respect to the point of attachment of said phenyl group to the rest of the molecule,
      wherein said heterocycloalkyl group is connected to the rest of the molecule via a carbon atom of said heterocycloalkyl group,
      wherein said phenyl, heteroaryl or heterocycloalkyl group is each optionally substituted with one or more substituents independently selected from a halogen atom, a C₁-C₃-alkyl group, a phenyl group and a R⁹OOC— group;
or a tautomer, or an N-oxide, or a salt thereof, or a salt of a tautomer, or a salt of an N-oxide, or a mixture of same.

In some embodiments, the present invention provides compounds of formula (I), supra, in which
R⁶ is selected from a phenyl group, a naphthyl group and a heteroaryl group,
   wherein said phenyl, naphthyl or heteroaryl group is each optionally substituted with one or more substituents independently selected from a halogen atom, a hydroxy group, a C₁-C₆-alkyl group, a C₁-C₆-hydroxyalkyl group, a C₁-C₆-haloalkyl group, a C₁-C₆-alkoxy group, a (C₁-C₃-alkyl)-O—(C₁-C₂-alkyl)- group, a (C₁-C₃-alkyl)-O—(C₁-C₂-alkyl)-O— group, a (C₃-C₃-cycloalkyl)-(C₁-C₂-alkyl)-O— group, a (C₃-C₃-halocycloalkyl)-(C₁-C₂-alkyl)-O— group, a (phenyl)-(C₁-C₂-alkyl)-O— group, a C₁-C₆-haloalkoxy group, a C₂-C₆-alkenyl group, a C₃-C₃-cycloalkyl group, a C₃-C₃-cycloalkoxy group, a C₁-C₆-thioalkyl group, a heterocycloalkyl group, a R⁷R⁸N— group, a (C₁-C₂-alkyl)-S(═O)₂— group, a (H₃C)—(C═O)— group, a (R⁷R⁸N)—(C═O)— group, a (R⁷R⁸N)—(C═O)—O— group, a R⁹OOC—(C₁-C₃-alkyl)- group, a R⁹OOC— group, a phenyl group, a (phenyl)-O— group and a heteroaryl group,
      wherein said heterocycloalkyl group is connected to the rest of the molecule via a carbon atom of said heterocycloalkyl group,
      wherein said phenyl, heteroaryl or heterocycloalkyl group is each optionally substituted with one or more substituents independently selected from a halogen atom and a C₁-C₃-alkyl group;
or a tautomer, or an N-oxide, or a salt thereof, or a salt of a tautomer, or a salt of an N-oxide, or a mixture of same.

In some embodiments, the present invention provides compounds of formula (I), supra, in which
R⁶ is selected from a phenyl group, a naphthyl group and a heteroaryl group,
   wherein said phenyl, naphthyl or heteroaryl group is each optionally substituted with one or more substituents independently selected from a halogen atom, a hydroxy group, a C₁-C₆-alkyl group, a C₁-C₆-hydroxyalkyl group, a C₁-C₆-haloalkyl group, a C₁-C₆-alkoxy group, a (C₁-C₃-alkyl)-O—(C₁-C₂-alkyl)- group, a (C₁-C₃-alkyl)-O—(C₁-C₂-alkyl)-O— group, a (C₃-C₃-cycloalkyl)-(C₁-C₂-alkyl)-O— group, a (C₃-C₃-halocycloalkyl)-(C₁-C₂-alkyl)-O— group, a (phenyl)-(C₁-C₂-alkyl)-O— group, a C₁-C₆-haloalkoxy group, a C₂-C₆-alkenyl group, a C₃-C₃-cycloalkyl group, a C₃-C₃-cycloalkoxy group, a C₁-C₆-thioalkyl group, a heterocycloalkyl group, a R⁷R⁸N— group, a (C₁-C₂-alkyl)-S(═O)₂— group, a (H₃C)—(C═O)— group, a (R⁷R⁸N)—(C═O)— group, a (R⁷R⁸N)—(C═O)—O— group, a R⁹OOC—(C₁-C₃-alkyl)- group, a R⁹OOC— group, a phenyl group, a (phenyl)-O— group and a heteroaryl group,
   and wherein, when substituted, said phenyl group is preferably substituted in one or two of the ortho-positions with respect to the point of attachment of said phenyl group to the rest of the molecule,
      wherein said heterocycloalkyl group is connected to the rest of the molecule via a carbon atom of said heterocycloalkyl group,
      wherein said phenyl, heteroaryl or heterocycloalkyl group is each optionally substituted with one or more substituents independently selected from a halogen atom and a C₁-C₃-alkyl group;
or a tautomer, or an N-oxide, or a salt thereof, or a salt of a tautomer, or a salt of an N-oxide, or a mixture of same.

In some embodiments, the present invention provides compounds of formula (I), supra, in which
R⁶ is selected from a phenyl group, a naphthyl group and a heteroaryl group,
   wherein said phenyl, naphthyl or heteroaryl group is each optionally substituted with one or more substituents independently selected from a halogen atom, a hydroxy group, a C₁-C₆-alkyl group, a C₁-C₆-hydroxyalkyl group, a C₁-C₆-haloalkyl group, a C₁-C₆-alkoxy group, a (C₁-C₃-alkyl)-O—(C₁-C₂-alkyl)- group, a (C₁-C₃-alkyl)-O—(C₁-C₂-alkyl)-O— group, a (C₃-C₃-cycloalkyl)-(C₁-C₂-alkyl)-O— group, a (C₃-C₃-halocycloalkyl)-(C₁-C₂-alkyl)-O— group, a (phenyl)-(C₁-C₂-alkyl)-O— group, a C₁-C₆-haloalkoxy group, a C₂-C₆-alkenyl group, a C₃-C₃-cycloalkyl group, a C₃-C₃-cycloalkoxy group, a C₁-C₆-thioalkyl group, a heterocycloalkyl group, a R⁷R⁸N— group, a (C₁-C₂-alkyl)-S(═O)₂— group, a (H₃C)—(C═O)— group, a (R⁷R⁸N)—(C═O)— group, a (R⁷R⁸N)—(C═O)—O— group, a R⁹OOC—(C₁-C₃-alkyl)- group, a R⁹OOC— group, a phenyl group, a (phenyl)-O— group and a heteroaryl group,
   and wherein, when substituted, said phenyl group is preferably substituted with a C₁-C₄-alkoxy group in one or two of the ortho-positions with respect to the point of attachment of said phenyl group to the rest of the molecule,
      wherein said heterocycloalkyl group is connected to the rest of the molecule via a carbon atom of said heterocycloalkyl group,
      wherein said phenyl, heteroaryl or heterocycloalkyl group is each optionally substituted with one or more substituents independently selected from a halogen atom and a C₁-C₃-alkyl group;
or a tautomer, or an N-oxide, or a salt thereof, or a salt of a tautomer, or a salt of an N-oxide, or a mixture of same.

In some embodiments, the present invention provides compounds of formula (I), supra, in which
R⁶ is selected from a phenyl group, a naphthyl group and a heteroaryl group,
   wherein said phenyl, naphthyl or heteroaryl group is each optionally substituted with one or more substituents independently selected from a halogen atom, a C₁-C₆-alkyl group, a C₁-C₆-haloalkyl group, a C₁-C₆-alkoxy group, a (C₁-C₃-alkyl)-O—(C₁-C₂-alkyl)- group, a (C₃-C₃-cycloalkyl)-(C₁-C₂-alkyl)-O— group, a (C₃-C₃-halocycloalkyl)-(C₁-C₂-alkyl)-O— group, a (phenyl)-(C₁-C₂-alkyl)-O— group, a C₁-C₆-haloalkoxy group, a C₂-C₆-alkenyl group, a C₃-C₃-cycloalkoxy group, a C₁-C₆-thioalkyl group, a heterocycloalkyl group, a R⁷R⁸N— group, a (C₁-C₂-alkyl)-S(═O)₂— group, a (R⁷R⁸N)—(C═O)— group, a (R⁷R⁸N)—(C═O)—O— group, a R⁹OOC—(C₁-C₃-alkyl)- group, a R⁹OOC— group, a phenyl group, a (phenyl)-O— group and a heteroaryl group,
      wherein said heterocycloalkyl group is connected to the rest of the molecule via a carbon atom of said heterocycloalkyl group,
      wherein said phenyl or heteroaryl group is each optionally substituted with one or more substituents independently selected from a halogen atom and a C₁-C₃-alkyl group;
or a tautomer, or an N-oxide, or a salt thereof, or a salt of a tautomer, or a salt of an N-oxide, or a mixture of same.

In some embodiments, the present invention provides compounds of formula (I), supra, in which
R⁶ is selected from a phenyl group, a naphthyl group and a heteroaryl group,
   wherein said phenyl, naphthyl or heteroaryl group is each optionally substituted with one or more substituents independently selected from a halogen atom, a C₁-C₆-alkyl group, a C₁-C₆-haloalkyl group, a C₁-C₆-alkoxy group, a (C₁-C₃-alkyl)-O—(C₁-C₂-alkyl)- group, a (C₃-C₃-cycloalkyl)-(C₁-C₂-alkyl)-O— group, a (C₃-C₃-halocycloalkyl)-(C₁-C₂-alkyl)-O— group, a (phenyl)-(C₁-C₂-alkyl)-O— group, a C₁-C₆-haloalkoxy group, a C₂-C₆-alkenyl group, a C₃-C₃-cycloalkoxy group, a C₁-C₆-thioalkyl group, a heterocycloalkyl group, a R⁷R⁸N— group, a (C₁-C₂-alkyl)-S(═O)₂— group, a (R⁷R⁸N)—(C═O)— group, a (R⁷R⁸N)—(C═O)—O— group, a R⁹OOC—(C₁-C₃-alkyl)- group, a R⁹OOC— group, a phenyl group, a (phenyl)-O— group and a heteroaryl group,
   and wherein, when substituted, said phenyl group is preferably substituted in one or two of the ortho-positions with respect to the point of attachment of said phenyl group to the rest of the molecule,
      wherein said heterocycloalkyl group is connected to the rest of the molecule via a carbon atom of said heterocycloalkyl group,
      wherein said phenyl or heteroaryl group is each optionally substituted with one or more substituents independently selected from a halogen atom and a C₁-C₃-alkyl group;
or a tautomer, or an N-oxide, or a salt thereof, or a salt of a tautomer, or a salt of an N-oxide, or a mixture of same.

In some embodiments, the present invention provides compounds of formula (I), supra, in which
R⁶ is selected from a phenyl group, a naphthyl group and a heteroaryl group,
   wherein said phenyl, naphthyl or heteroaryl group is each optionally substituted with one or more substituents independently selected from a halogen atom, a C₁-C₆-alkyl group, a C₁-C₆-haloalkyl group, a C₁-C₆-alkoxy group, a (C₁-C₃-alkyl)-O—(C₁-C₂-alkyl)- group, a (C₃-C₃-cycloalkyl)-(C₁-C₂-alkyl)-O— group, a (C₃-C₃-halocycloalkyl)-(C₁-C₂-alkyl)-O— group, a (phenyl)-(C₁-C₂-alkyl)-O— group, a C₁-C₆-haloalkoxy group, a C₂-C₆-alkenyl group, a C₃-C₃-cycloalkoxy group, a C₁-C₆-thioalkyl group, a heterocycloalkyl group, a R⁷R⁸N— group, a (C₁-C₂-alkyl)-S(═O)₂— group, a (R⁷R⁸N)—(C═O)— group, a (R⁷R⁸N)—(C═O)—O— group, a R⁹OOC—(C₁-C₃-alkyl)- group, a R⁹OOC— group, a phenyl group, a (phenyl)-O— group and a heteroaryl group,
   and wherein, when substituted, said phenyl group is preferably substituted with a C₁-C₄-alkoxy group in one or two of the ortho-positions with respect to the point of attachment of said phenyl group to the rest of the molecule,
      wherein said heterocycloalkyl group is connected to the rest of the molecule via a carbon atom of said heterocycloalkyl group,
      wherein said phenyl or heteroaryl group is each optionally substituted with one or more substituents independently selected from a halogen atom and a C₁-C₃-alkyl group;
or a tautomer, or an N-oxide, or a salt thereof, or a salt of a tautomer, or a salt of an N-oxide, or a mixture of same.

In some embodiments, the present invention provides compounds of formula (I), supra, in which
R⁶ is selected from a phenyl group, a naphthyl group and a heteroaryl group,
   wherein said phenyl, naphthyl or heteroaryl group is each optionally substituted with one or more substituents independently selected from a halogen atom, a C₁-C₆-alkyl group, a C₁-C₆-haloalkyl group, a C₁-C₆-alkoxy group, a (C₁-C₃-alkyl)-O—(C₁-C₂-alkyl)- group, a (C₁-C₃-alkyl)-O—(C₁-C₂-alkyl)-O— group, a C₂-C₆-alkenyl group, a C₁-C₆-thioalkyl group, a heterocycloalkyl group, a R⁷R⁸N— group, a (C₁-C₂-alkyl)-S(═O)₂— group, a (R⁷R⁸N)—(C═O)— group, a (R⁷R⁸N)—(C═O)—O— group a R⁹OOC—(C₁-C₃-alkyl)- group, a R⁹OOC— group, and a heteroaryl group,
      wherein said heterocycloalkyl group is connected to the rest of the molecule via a carbon atom of said heterocycloalkyl group,
      wherein said heteroaryl group is each optionally substituted with one or more substituents independently selected from a C₁-C₆-alkyl group;
or a tautomer, or an N-oxide, or a salt thereof, or a salt of a tautomer, or a salt of an N-oxide, or a mixture of same.

In some embodiments, the present invention provides compounds of formula (I), supra, in which
R⁶ is selected from a phenyl group, a naphthyl group and a heteroaryl group,
   wherein said phenyl, naphthyl or heteroaryl group is each optionally substituted with one or more substituents independently selected from a halogen atom, a C₁-C₆-alkyl group, a C₁-C₆-haloalkyl group, a C₁-C₆-alkoxy group, a (C₁-C₃-alkyl)-O—(C₁-C₂-alkyl)- group, a (C₁-C₃-alkyl)-O—(C₁-C₂-alkyl)-O— group, a C₂-C₆-alkenyl group, a C₁-C₆-thioalkyl group, a heterocycloalkyl group, a R⁷R⁸N— group, a (C₁-C₂-alkyl)-S(═O)₂— group, a (R⁷R⁸N)—(C═O)— group, a (R⁷R⁸N)—(C═O)—O— groupa R⁹OOC—(C₁-C₃-alkyl)- group, a R⁹OOC— group, and a heteroaryl group,
   and wherein, when substituted, said phenyl group is preferably substituted in one or two of the ortho-positions with respect to the point of attachment of said phenyl group to the rest of the molecule,
      wherein said heterocycloalkyl group is connected to the rest of the molecule via a carbon atom of said heterocycloalkyl group,
      wherein said heteroaryl group is each optionally substituted with one or more substituents independently selected from a C₁-C₆-alkyl group;
or a tautomer, or an N-oxide, or a salt thereof, or a salt of a tautomer, or a salt of an N-oxide, or a mixture of same.

In some embodiments, the present invention provides compounds of formula (I), supra, in which
R⁶ is selected from a phenyl group, a naphthyl group and a heteroaryl group,
   wherein said phenyl, naphthyl or heteroaryl group is each optionally substituted with one or more substituents independently selected from a halogen atom, a C₁-C₆-alkyl group, a C₁-C₆-haloalkyl group, a C₁-C₆-alkoxy group, a (C₁-C₃-alkyl)-O—(C₁-C₂-alkyl)- group, a (C₁-C₃-alkyl)-O—(C₁-C₂-alkyl)-O— group, a C₂-C₆-alkenyl group, a C₁-C₆-thioalkyl group, a heterocycloalkyl group, a R⁷R⁸N— group, a (C₁-C₂-alkyl)-S(═O)₂— group, a (R⁷R⁸N)—(C═O)— group, a (R⁷R⁸N)—(C═O)—O— groupa R⁹OOC—(C₁-C₃-alkyl)- group, a R⁹OOC— group, and a heteroaryl group,
   and wherein, when substituted, said phenyl group is preferably substituted with a C₁-C₄-alkoxy group in one or two of the ortho-positions with respect to the point of attachment of said phenyl group to the rest of the molecule,
      wherein said heterocycloalkyl group is connected to the rest of the molecule via a carbon atom of said heterocycloalkyl group,
      wherein said heteroaryl group is each optionally substituted with one or more substituents independently selected from a C₁-C₆-alkyl group;
or a tautomer, or an N-oxide, or a salt thereof, or a salt of a tautomer, or a salt of an N-oxide, or a mixture of same.

In some embodiments, the present invention provides compounds of formula (I), supra, in which
R⁶ is selected from a phenyl group and a heteroaryl group,
   wherein said phenyl or heteroaryl group is each optionally substituted with one or more substituents independently selected from a halogen atom, a C₁-C₆-alkyl group, a C₁-C₆-alkoxy group, a (phenyl)-(C₁-C₂-alkyl)-O— group, a C₁-C₆-haloalkoxy group, a C₂-C₆-alkenyl group, a C₃-C₃-cycloalkoxy group, a heterocycloalkyl group and a phenyl group,
      wherein said heterocycloalkyl group is connected to the rest of the molecule via a carbon atom of said heterocycloalkyl group,
      wherein said phenyl, or heterocycloalkyl group is each optionally substituted with one or more substituents independently selected from a halogen atom, a C₁-C₃-alkyl group and a R⁹OOC— group;
or a tautomer, or an N-oxide, or a salt thereof, or a salt of a tautomer, or a salt of an N-oxide, or a mixture of same.

In some embodiments, the present invention provides compounds of formula (I), supra, in which
R⁶ is selected from a phenyl group and a heteroaryl group,
   wherein said phenyl or heteroaryl group is each optionally substituted with one or more substituents independently selected from a halogen atom, a C₁-C₆-alkyl group, a C₁-C₆-alkoxy group, a (phenyl)-(C₁-C₂-alkyl)-O— group, a C₁-C₆-haloalkoxy group, a C₂-C₆-alkenyl group, a C₃-C₃-cycloalkoxy group, a heterocycloalkyl group and a phenyl group,
   and wherein, when substituted, said phenyl group is preferably substituted in one or two of the ortho-positions with respect to the point of attachment of said phenyl group to the rest of the molecule,
      wherein said heterocycloalkyl group is connected to the rest of the molecule via a carbon atom of said heterocycloalkyl group,
      wherein said phenyl, or heterocycloalkyl group is each optionally substituted with one or more substituents independently selected from a halogen atom, a C₁-C₃-alkyl group and a R⁹OOC— group;
or a tautomer, or an N-oxide, or a salt thereof, or a salt of a tautomer, or a salt of an N-oxide, or a mixture of same.

In some embodiments, the present invention provides compounds of formula (I), supra, in which
R⁶ is selected from a phenyl group and a heteroaryl group,
   wherein said phenyl or heteroaryl group is each optionally substituted with one or more substituents independently selected from a halogen atom, a C₁-C₆-alkyl group, a C₁-C₆-alkoxy group, a (phenyl)-(C₁-C₂-alkyl)-O— group, a C₁-C₆-haloalkoxy group, a C₂-C₆-alkenyl group, a C₃-C₃-cycloalkoxy group, a heterocycloalkyl group and a phenyl group,
   and wherein, when substituted, said phenyl group is preferably substituted with a C₁-C₄-alkoxy group in one or two of the ortho-positions with respect to the point of attachment of said phenyl group to the rest of the molecule,
      wherein said heterocycloalkyl group is connected to the rest of the molecule via a carbon atom of said heterocycloalkyl group,
      wherein said phenyl, or heterocycloalkyl group is each optionally substituted with one or more substituents independently selected from a halogen atom, a C₁-C₃-alkyl group and a R⁹OOC— group;
or a tautomer, or an N-oxide, or a salt thereof, or a salt of a tautomer, or a salt of an N-oxide, or a mixture of same.

In some embodiments, the present invention provides compounds of formula (I), supra, in which
R⁶ is selected from a phenyl group and a heteroaryl group,
   wherein said phenyl or heteroaryl group is each optionally substituted with one or more substituents independently selected from a halogen atom, a C₁-C₆-alkyl group, a C₁-C₆-haloalkyl group, a C₁-C₆-alkoxy group, a (C₃-C₃-cycloalkyl)-(C₁-C₂-alkyl)-O— group, a (C₃-C₃-halocycloalkyl)-(C₁-C₂-alkyl)-O— group, a (phenyl)-(C₁-C₂-alkyl)-O— group, a C₁-C₆-haloalkoxy group, a C₃-C₃-cycloalkoxy group, a heterocycloalkyl group, a R⁷R⁸N— group, a (phenyl)-O— group and a heteroaryl group,
      wherein said heterocycloalkyl group is connected to the rest of the molecule via a carbon atom of said heterocycloalkyl group,
      wherein said heteroaryl or heterocycloalkyl group is each optionally substituted with one or more substituents independently selected from a C₁-C₃-alkyl group and a phenyl group;
or a tautomer, or an N-oxide, or a salt thereof, or a salt of a tautomer, or a salt of an N-oxide, or a mixture of same.

In some embodiments, the present invention provides compounds of formula (I), supra, in which
R⁶ is selected from a phenyl group and a heteroaryl group,
   wherein said phenyl or heteroaryl group is each optionally substituted with one or more substituents independently selected from a halogen atom, a C₁-C₆-alkyl group, a C₁-C₆-haloalkyl group, a C₁-C₆-alkoxy group, a (C₃-C₃-cycloalkyl)-(C₁-C₂-alkyl)-O— group, a (C₃-C₃-halocycloalkyl)-(C₁-C₂-alkyl)-O— group, a (phenyl)-(C₁-C₂-alkyl)-O— group, a C₁-C₆-haloalkoxy group, a C₃-C₃-cycloalkoxy group, a heterocycloalkyl group, a R⁷R⁸N— group, a (phenyl)-O— group and a heteroaryl group,
   and wherein, when substituted, said phenyl group is preferably substituted in one or two of the ortho-positions with respect to the point of attachment of said phenyl group to the rest of the molecule,
      wherein said heterocycloalkyl group is connected to the rest of the molecule via a carbon atom of said heterocycloalkyl group,
      wherein said heteroaryl or heterocycloalkyl group is each optionally substituted with one or more substituents independently selected from a C₁-C₃-alkyl group and a phenyl group;
or a tautomer, or an N-oxide, or a salt thereof, or a salt of a tautomer, or a salt of an N-oxide, or a mixture of same.

In some embodiments, the present invention provides compounds of formula (I), supra, in which
R⁶ is selected from a phenyl group and a heteroaryl group,
   wherein said phenyl or heteroaryl group is each optionally substituted with one or more substituents independently selected from a halogen atom, a C₁-C₆-alkyl group, a C₁-C₆-haloalkyl group, a C₁-C₆-alkoxy group, a (C₃-C₃-cycloalkyl)-(C₁-C₂-alkyl)-O— group, a (C₃-C₃-halocycloalkyl)-(C₁-C₂-alkyl)-O— group, a (phenyl)-(C₁-C₂-alkyl)-O— group, a C₁-C₆-haloalkoxy group, a C₃-C₃-cycloalkoxy group, a heterocycloalkyl group, a R⁷R⁸N— group, a (phenyl)-O— group and a heteroaryl group,
   and wherein, when substituted, said phenyl group is preferably substituted with a C₁-C₄-alkoxy group in one or two of the ortho-positions with respect to the point of attachment of said phenyl group to the rest of the molecule,
      wherein said heterocycloalkyl group is connected to the rest of the molecule via a carbon atom of said heterocycloalkyl group,
      wherein said heteroaryl or heterocycloalkyl group is each optionally substituted with one or more substituents independently selected from a C₁-C₃-alkyl group and a phenyl group;
or a tautomer, or an N-oxide, or a salt thereof, or a salt of a tautomer, or a salt of an N-oxide, or a mixture of same.

In some embodiments, the present invention provides compounds of formula (I), supra, in which
R⁷ and R³ are each independently selected from a hydrogen atom, a C₁-C₆-alkyl group, a C₃-C₃-cycloalkyl group, a C₁-C₆-haloalkyl group, a (C₁-C₆-alkyl)C(═O)— group, a (C₃-C₃-cycloalkyl)-(C₁-C₆-alkyl)- group, a (C₁-C₆-alkoxy)-(C₁-C₆-alkyl)- group, a phenyl group, and a heterocycloalkyl group,
   wherein said heterocycloalkyl group is connected to the rest of the molecule via a carbon atom of said heterocycloalkyl group,
   wherein said phenyl or heterocycloalkyl group is each optionally substituted with one or more substituents independently selected from a halogen atom, a C₁-C₃-alkyl group, a C₁-C₃-haloalkyl group and a C₁-C₃-alkoxy group;
or R⁷ and R^{a} together with the nitrogen atom to which they are attached form a 4- to 7-membered nitrogen-containing heterocycloalkyl group, wherein said heterocycloalkyl group optionally contains one, two or three further heteroatoms independently selected from nitrogen, oxygen and sulfur and is optionally substituted with one or more substituents independently selected from a halogen atom, a C₁-C₃-alkyl group, a C₁-C₃-haloalkyl group, a C₁-C₃-alkoxy group and a phenyl group;
or a tautomer, or an N-oxide, or a salt thereof, or a salt of a tautomer, or a salt of an N-oxide, or a mixture of same.

In some embodiments, the present invention provides compounds of formula (I), supra, in which
R⁷ and R³ are each independently selected from a hydrogen atom, a C₁-C₆-alkyl group, a C₃-C₃-cycloalkyl group and a phenyl group,
   wherein said phenyl group is optionally substituted with one or more substituents independently selected from a halogen atom, a C₁-C₃-alkyl group, a C₁-C₃-haloalkyl group and a C₁-C₃-alkoxy group;
or R⁷ and R^{a} together with the nitrogen atom to which they are attached form a 4- to 7-membered nitrogen-containing heterocycloalkyl group, wherein said heterocycloalkyl group optionally contains one, two or three further heteroatoms independently selected from nitrogen, oxygen and sulfur and is optionally substituted with one or more substituents independently selected from a halogen atom, a C₁-C₃-alkyl group, a C₁-C₃-haloalkyl group, a C₁-C₃-alkoxy group and a phenyl group;
or a tautomer, or an N-oxide, or a salt thereof, or a salt of a tautomer, or a salt of an N-oxide, or a mixture of same.

In some embodiments, the present invention provides compounds of formula (I), supra, in which
R⁷ and R⁸ are each independently selected from a hydrogen atom, a C₁-C₆-alkyl group, a C₃-C₃-cycloalkyl group and a phenyl group,
   wherein said phenyl group is optionally substituted with one or more substituents independently selected from a halogen atom, a C₁-C₃-alkyl group, a C₁-C₃-haloalkyl group and a C₁-C₃-alkoxy group;
or a tautomer, or an N-oxide, or a salt thereof, or a salt of a tautomer, or a salt of an N-oxide, or a mixture of same.

In some embodiments, the present invention provides compounds of formula (I), supra, in which
R⁷ and R^{a} together with the nitrogen atom to which they are attached form a 4- to 7-membered nitrogen-containing heterocycloalkyl group, wherein said heterocycloalkyl group optionally contains one, two or three further heteroatoms independently selected from nitrogen, oxygen and sulfur and is optionally substituted with one or more substituents independently selected from a halogen atom, a C₁-C₃-alkyl group, a C₁-C₃-haloalkyl group, a C₁-C₃-alkoxy group and a phenyl group;
or a tautomer, or an N-oxide, or a salt thereof, or a salt of a tautomer, or a salt of an N-oxide, or a mixture of same.

In some embodiments, the present invention provides compounds of formula (I), supra, in which R⁷ and R⁸ are each independently selected from a hydrogen atom, a C₁-C₄-alkyl group and a C₃-C₃-cycloalkyl group or a tautomer, or an N-oxide, or a salt thereof, or a salt of a tautomer, or a salt of an N-oxide, or a mixture of same.

In some embodiments, the present invention provides compounds of formula (I), supra, in which R⁷ and R⁸ are each independently selected from a hydrogen atom and a C₁-C₄-alkyl group or a tautomer, or an N-oxide, or a salt thereof, or a salt of a tautomer, or a salt of an N-oxide, or a mixture of same.

In some embodiments, the present invention provides compounds of formula (I), supra, in which R⁷ and R³ are each independently selected from a hydrogen atom and a methyl group or a tautomer, or an N-oxide, or a salt thereof, or a salt of a tautomer, or a salt of an N-oxide, or a mixture of same.

In some embodiments, the present invention provides compounds of formula (I), supra, in which
R^{a} and R^{b} together with the nitrogen atom to which they are attached form a 4-membered nitrogen-containing heterocycloalkyl group which is optionally substituted one, two or three times, each substituent independently selected from a halogen atom, a C₁-C₂-alkyl group, a C₁-C₂-haloalkyl group, a C₁-C₂-alkoxy group, a C₁-C₃-hydroxyalkyl group and a phenyl group
or a tautomer, or an N-oxide, or a salt thereof, or a salt of a tautomer, or a salt of an N-oxide, or a mixture of same.

In some embodiments, the present invention provides compounds of formula (I), supra, in which
R^{a} and R^{b} together with the nitrogen atom to which they are attached form a 4-membered nitrogen-containing heterocycloalkyl group which is optionally substituted one, two or three times, each substituent independently selected from a halogen atom, a C₁-C₂-haloalkyl group and a C₁-C₃-hydroxyalkyl group
or a tautomer, or an N-oxide, or a salt thereof, or a salt of a tautomer, or a salt of an N-oxide, or a mixture of same.

In some embodiments, the present invention provides compounds of formula (I), supra, in which
R^{a} and R^{b} together with the nitrogen atom to which they are attached form a 4-membered nitrogen-containing heterocycloalkyl group which is optionally substituted one time with a fluorine atom
or a tautomer, or an N-oxide, or a salt thereof, or a salt of a tautomer, or a salt of an N-oxide, or a mixture of same.

In some embodiments, the present invention provides compounds of formula (I), supra, in which
R^{a} and R^{b} together with the nitrogen atom to which they are attached form a 4-membered nitrogen-containing heterocycloalkyl group which is optionally substituted one, two or three times with a halogen atom
or a tautomer, or an N-oxide, or a salt thereof, or a salt of a tautomer, or a salt of an N-oxide, or a mixture of same.

In some embodiments, the present invention provides compounds of formula (I), supra, in which
R^{a} and R^{b} together with the nitrogen atom to which they are attached form an unsubstituted 4- membered nitrogen-containing heterocycloalkyl group
or a tautomer, or an N-oxide, or a salt thereof, or a salt of a tautomer, or a salt of an N-oxide, or a mixture of same.

In some embodiments, the present invention provides compounds of formula (I), supra, in which R⁹ is selected from a hydrogen atom, a C₁-C₃-alkyl group and a C₃-C₅-cycloalkyl group or a tautomer, or an N-oxide, or a salt thereof, or a salt of a tautomer, or a salt of an N-oxide, or a mixture of same.

In some embodiments, the present invention provides compounds of formula (I), supra, in which R⁹ is selected from a hydrogen atom and a C₁-C₃-alkyl group or a tautomer, or an N-oxide, or a salt thereof, or a salt of a tautomer, or a salt of an N-oxide, or a mixture of same.

In some embodiments, the present invention provides compounds of formula (I), supra, in which R⁹ is a hydrogen atom or a tautomer, or an N-oxide, or a salt thereof, or a salt of a tautomer, or a salt of an N-oxide, or a mixture of same.

In some embodiments, the present invention provides compounds of formula (I), supra, in which R⁹ is a C₁-C₃-alkyl group or a tautomer, or an N-oxide, or a salt thereof, or a salt of a tautomer, or a salt of an N-oxide, or a mixture of same.

In some embodiments, the present invention provides compounds of formula (I), supra, in which R⁹ is a C₃-C₅-cycloalkyl group or a tautomer, or an N-oxide, or a salt thereof, or a salt of a tautomer, or a salt of an N-oxide, or a mixture of same.

In further embodiments, the present invention includes compounds of formula (I), or a tautomer, an N-oxide, or a salt thereof, or a salt of a tautomer or an N-oxide, or a mixture of same.

In further embodiments, the present invention includes compounds of formula (I), or a salt thereof.

In further embodiments, the present invention includes compounds of formula (I), or a tautomer, or a salt thereof, or a salt of a tautomer, or a mixture of same.

In further embodiments, the present invention includes compounds of formula (I), which are a salt.

In further embodiments, the present invention includes compounds of formula (I), which are a tautomer or a salt thereof, or a salt of a tautomer, or a mixture of same.

In further embodiments, the present invention includes compounds of formula (I), which are an N-oxide, or a salt thereof, or a salt of an N-oxide, or a mixture of same.

In further embodiments of the first aspect, the present invention provides combinations of two or more of the above mentioned embodiments under the heading “further embodiments of the first aspect of the present invention”.

Furthermore it is understood that the invention includes any subcombination of the disclosed single embodiments herein for certain residues or subcombination of residues of formula (I).

The present invention includes any sub-combination within any embodiments or aspects of the present invention of compounds of general formula (I), supra.

The present invention includes any sub-combination within any embodiments or aspects of the present invention of compounds of general formula (I) or intermediate compounds.

The present invention includes the compounds of general formula (I) which are disclosed in the Example Section of this text, infra.

### General Synthesis of Compounds of General Formula (I) of the Present Invention

The compounds of general formula (I) according to the invention as well as relevant intermediate and/or precursor compounds can be prepared according to the following schemes 1, 2, 3, 4, 5 and 6. The schemes and procedures described below illustrate synthetic routes to the compounds of general formula (I) of the invention as well as to relevant intermediate and/or precursor compounds and are not intended to be limiting. It is clear to the person skilled in the art that the order of transformations as exemplified in schemes 1, 2, 3, 4, 5 and 6 can be modified in various ways. The order of transformations exemplified in these schemes is therefore not intended to be limiting. In addition, interconversion of any of the substituents R¹, R², R³, R⁴, R⁵, R⁶, R^{a} and R^{b} can often be achieved before and/or after the exemplified transformations. These modifications can include the introduction of protecting groups, cleavage of protecting groups, reduction or oxidation of functional groups, halogenation, metallation, substitution or other suitable reactions known to the person skilled in the art. These transformations include those which introduce a functionality which allows for further interconversion of substituents. Appropriate protecting groups and their introduction and cleavage are well-known to the person skilled in the art (see for example T. W. Greene and P. G. M. Wuts in Protective Groups in Organic Synthesis, 3^{rd} edition, Wiley 1999). Specific examples are described in the subsequent paragraphs and in the Experimental Section.

Scheme 1 describes one possible route for the preparation of compounds of general formula (I), in which R¹, R², R³, R⁴, R⁵, R⁶, R^{a} and R^{b} have the meaning as given for the compounds of general formula (I), supra.

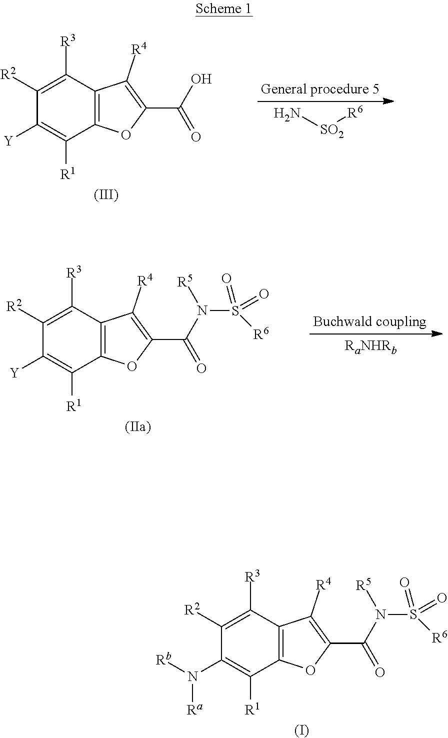

As depicted in Scheme 1, compounds of general formula (I) can be synthesized via Buchwald-Hartwig amination of a derivative of formula (IIa) with a suitable amine R^{a}NHR^{b}, wherein R¹, R², R³, R⁴, R⁵, R⁶. R^{a} and R^{b} have the meaning as given for general formula (I), supra and Y is a halogen atom, such as e.g. fluorine, chlorine, bromine or iodine, preferably chlorine, bromine or iodine, more preferably chlorine or bromine. For the Buchwald-Hartwig amination reactions described in the context of the present invention, see, for example: Chem Sci. 2011, 2, 27-50; RSC Adv., 2017, 7, 51972-51977. Specific examples are described in the Experimental Section. Said Buchwald-Hartwig amination reactions are carried out in the presence of a catalyst, preferably in the presence of a second- or third-generation Buchwald precatalyst (G2 or G3), which are widely known to the skilled person and/or commercially available. The reaction is preferably carried out in a solvent such as e.g. dimethyl formamide.

In turn, derivatives of formula (IIa) can be prepared via the coupling of an intermediate of formula (III) with a primary sulfonamide of formula H₂N—S(═O)₂—R⁶, wherein R⁶ has the meaning as given for the compounds of general formula (I), supra. For the coupling of sulfonamides in the context of the present invention see, for example: Org. Proc. Res. Dev., 2009, 13, 255-262; Angew. Chem. Int. Ed. 2018, 57, 3488-3492. Specific examples are described in the Experimental Section.

Depending inter alia on the nature of the substituents R¹, R², R³, R⁴, R⁵, R⁶, R^{a} and R^{b}, compounds of general formula (I) can also be prepared via intermediates of formula (IIb), in which the —NR^{a}R^{b} moiety is introduced before the coupling with a primary sulfonamide of formula H₂N—S(═O)₂—R⁶, wherein R⁶ has the meaning as given for the compounds of general formula (I), supra (Scheme 2). Said compounds of formula (IIb) can be prepared via Buchwald-Hartwig amination starting from compounds of formula (III), wherein Y is a halogen atom, such as e.g. fluorine, chlorine, bromine or iodine, preferably chlorine, bromine or iodine, more preferably chlorine or bromine. Specific examples are described in the Experimental Section.

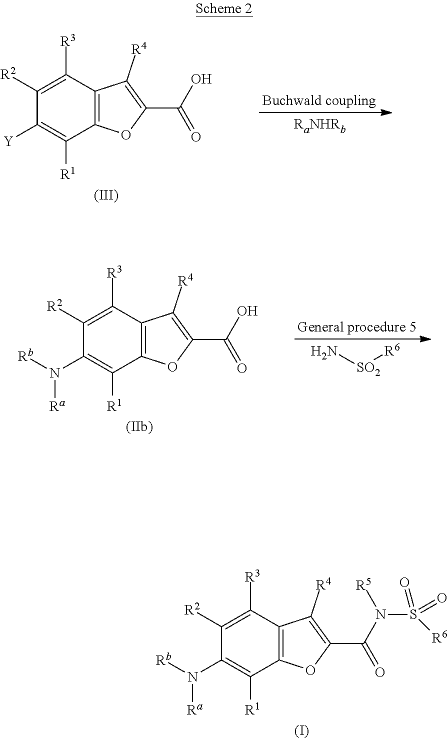

In general, the synthesis of the compounds of formula (I) of the present invention is preferably carried out following the sequence depicted in Scheme 2.

The compounds of formula (IIa) and/or (IIb) and/or (III) are either commercially available or can be prepared according to procedures available from the public domain, as understandable to the person skilled in the art. Specific examples are described in the Experimental Section.

For example, the benzofuran derivatives of formula (III) wherein R¹, R², R³ and R⁴ have the meaning as given for the compounds of general formula (I), supra and Y is a halogen atom, such as e.g. fluorine, chlorine, bromine or iodine, preferably chlorine, bromine or iodine, more preferably chlorine or bromine, can be prepared via the reaction between a hydroxybenzaldehyde of formula (IVa) and for example ethyl chloro- or bromoacetate or any other suitable reagent of similar nature (Scheme 3). It can happen that mixtures of the free carboxylic acid and the corresponding ester are obtained. In this case, a saponification step might be necessary, which can be carried out using standard methods known in the art. Analogously and alternatively, compounds of formula (IIb) can be prepared starting from compounds of formula (IVb) wherein R¹, R², R³, R⁴, R^{a} and R^{b} have the meaning as given for the compounds of general formula (I), supra and Y is a halogen atom, such as e.g. fluorine, chlorine, bromine or iodine, preferably chlorine, bromine or iodine, more preferably chlorine or bromine (Scheme 4). For the cyclization reactions described herein, see, for example: Chem. Biol. Drug. Des., 2018, 92, 1497-1503; J. of enzyme inh. Med. Chem. 2018, 33, 1, 1212-1224. Specific examples are described in the Experimental Section.

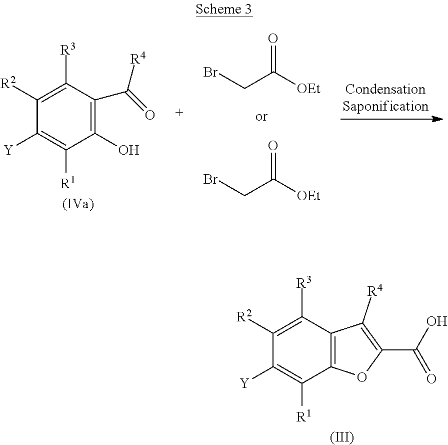

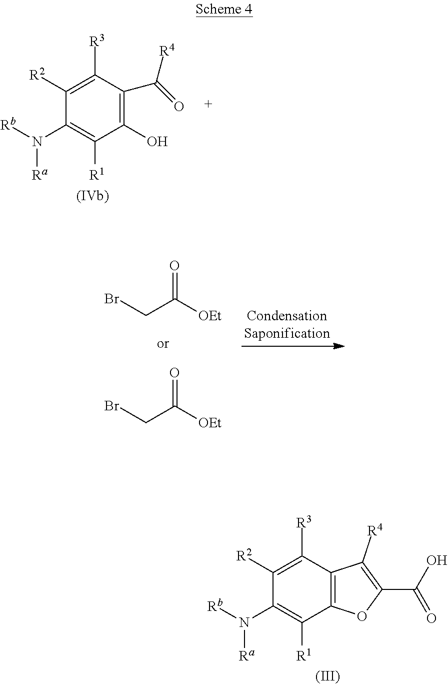

The sulfonamides of formula H₂N—S(═O)₂—R⁶ are either commercially available or can be prepared according to procedures available from the public domain, as understandable to the person skilled in the art. Specific examples are described in the Experimental Section.

The compounds of formula (IVb) wherein R³ is fluorine and R⁴ is H can be prepared by aromatic nucleophilic substitution using a suitable amine R^{a}R^{b}NH or its hydrochloride salt wherein R^{a} and R^{b} have the meaning as given for the compounds of general formula (I), supra and a base such as N,N-Diisopropylethylamine according to Scheme 5 according to procedures available from the public domain, and known to the person skilled in the art.

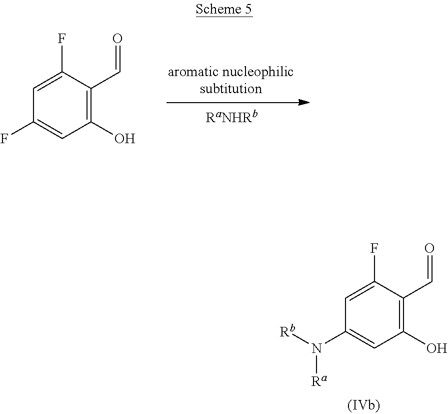

For example, suitable sulfonamides comprising a (phenyl)-(phenyl)- moiety can be prepared via the sequence depicted in Scheme 6, comprising the protection of an aryl sulfonamide with 1,1-dimethoxy-N,N-dimethylmethanamine, a coupling reaction with a suitable boronic acid derivative and optional deprotection, if necessary. The phenyl moieties can each be individually unsubstituted or substituted as defined for the compounds of general formula (I), supra. [B] indicates a boron-containing functionality suitable for the carbon-carbon coupling of the two phenyl moieties, e.g. a group —B(OR^{B})₂ wherein OR^{B} is a substituent that, when attached to the boron atom, can be used as coupling partner in a Suzuki reaction. For the reaction of aryl sulfonamides with 1,1-dimethoxy-N,N-dimethylmethanamine see, for example: Organic Preparations and Procedures International 2002, 34(5), 545-549; WO2005/26158; Green Chemistry 2013, 15(8), 2294-2301. Specific examples are described in the Experimental Section.

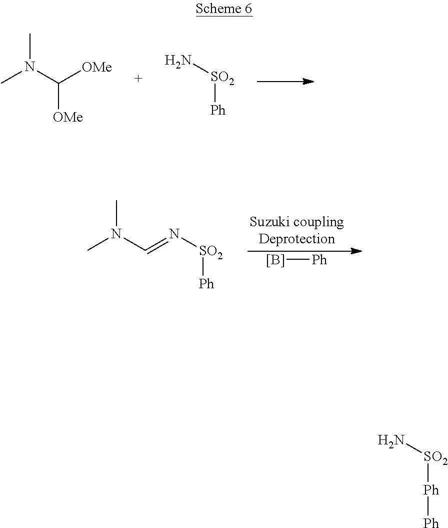

Further, for the compounds of formula (I) wherein R⁴ is as defined for the compounds of general formula (I), supra but not a hydrogen atom, the corresponding substituent can be already be incorporated in one of the starting materials or if necessary it can be introduced at different stages during the reaction sequence leading to the compounds of formula (I). This can be achieved using methods, reagents and conditions well-known to the person skilled in the art. Specific examples are described in the Experimental Section.

In accordance with a second aspect, the present invention provides methods of preparing compounds of general formula (I) as defined supra, said method comprising the reaction of an intermediate compound of formula (IIa)

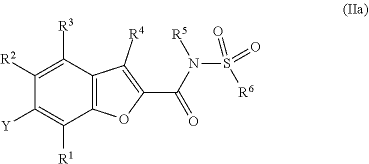

with an amine, wherein R¹, R², R³, R⁴, R⁵ and R⁶ are as defined for the compounds of general formula (I) supra, or a tautomer, or an N-oxide, or a salt thereof, or a salt of a tautomer, or a salt of an N-oxide, or a mixture of same and Y is a halogen atom selected from fluorine, chlorine, bromine or iodine, preferably chlorine, bromine or iodine, more preferably chlorine or bromine, thereby giving a compound of general formula (I)

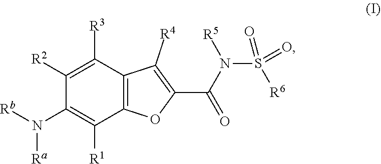

wherein R¹, R², R³, R⁴, R⁵, R⁶, R^{a} and R^{b} are as defined supra, or a tautomer, or an N-oxide, or a salt thereof, or a salt of a tautomer, or a salt of an N-oxide, or a mixture of same.

Further, the present invention provides intermediate compounds of general formula (IIa)

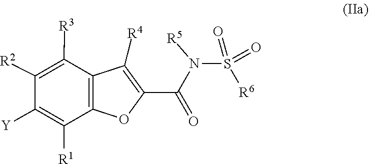

wherein R¹, R², R³, R⁴, R⁵ and R⁶ are as defined for the compounds of general formula (I) supra and Y is a halogen atom selected from chlorine and bromine or a tautomer, or an N-oxide, or a salt thereof, or a salt of a tautomer, or a salt of an N-oxide, or a mixture of same.

Particularly, the present invention provides the use of intermediate compounds of general formula (IIa)

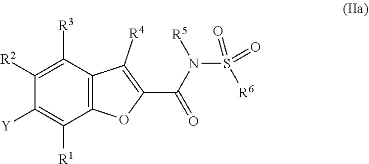

wherein R¹, R², R³, R⁴, R⁵ and R⁶ are as defined for the compounds of general formula (I) supra, or a tautomer, or an N-oxide, or a salt thereof, or a salt of a tautomer, or a salt of an N-oxide, or a mixture of same and Y is a halogen atom selected from fluorine, chlorine, bromine or iodine, preferably chlorine, bromine or iodine, more preferably chlorine or bromine, for the preparation of a compound of general formula (I) as defined supra.

In accordance with a third aspect, the present invention provides methods of preparing compounds of general formula (I) as defined supra, said method comprising the reaction of an intermediate compound of formula (IIb)

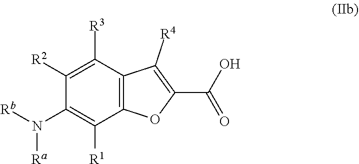

with an sulfonamide of general formula H₂N—SO₂—R⁶, wherein R¹, R², R³, R⁴, R⁶, R^{a} and R^{b} are as defined for the compounds of general formula (I) supra, or a tautomer, or an N-oxide, or a salt thereof, or a salt of a tautomer, or a salt of an N-oxide, or a mixture of same, thereby giving a compound of general formula (I)

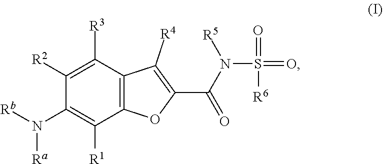

wherein R¹, R², R³, R⁴, R⁶, R^{a} and R^{b} are as defined supra and R⁵ is a hydrogen atom, or a tautomer, or an N-oxide, or a salt thereof, or a salt of a tautomer, or a salt of an N-oxide, or a mixture of same.

Further, the present invention provides intermediate compounds of general formula (IIb)

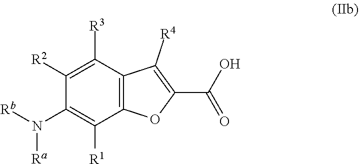

wherein R¹, R², R³, R⁴, R^{a} and R^{b} are as defined for the compounds of general formula (I) supra and R⁵ is a hydrogen atom, or a tautomer, or an N-oxide, or a salt thereof, or a salt of a tautomer, or a salt of an N-oxide, or a mixture of same.

Particularly, the present invention provides the use of intermediate compounds of general formula (IIb)

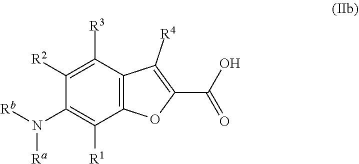

wherein R¹, R², R³, R⁴, R^{a} and R^{b} are as defined for the compounds of general formula (I) supra, or a tautomer, or an N-oxide, or a salt thereof, or a salt of a tautomer, or a salt of an N-oxide, or a mixture of same for the preparation of a compound of general formula (I) as defined supra.

The present invention includes the intermediate compounds which are disclosed in the Example Section of this text, infra.

The present invention includes any sub-combination within any embodiments or aspects of the present invention of intermediate compounds of general formula (IIa), of general formula (IIb), of general formula (III), of general formula (IVa), of general formula (IVb), supra.

The compounds of general formula (I) of the present invention can be converted to any salt, preferably pharmaceutically acceptable salts, by any method which is known to the person skilled in the art. Similarly, any salt of a compound of general formula (I) of the present invention can be converted into the free compound, by any method which is known to the person skilled in the art.

Compounds of general formula (I) of the present invention demonstrate a valuable pharmacological spectrum of action which could not have been predicted. The compounds of the present invention effectively inhibit the activity of lysine acetyl transferase 6A (KAT6A) and/or lysine acetyl transferase 6B (KAT6B) for which data are given in the biological experimental section and may therefore be used for the treatment or prophylaxis of hyperproliferative disorders, such as cancer disorders in humans and animals.

### Methods and Administration

Compounds of general formula (I) of the present invention demonstrate a valuable pharmacological spectrum of action and pharmacokinetic profile, both of which could not have been predicted. Compounds of the present invention have surprisingly been found to effectively inhibit the activity of lysine acetyl transferase 6A (KAT6A) and lysine acetyl transferase 6B (KAT6B), and it is possible therefore that said compounds can be used for the treatment or prophylaxis of diseases, preferably hyperproliferative disorders in humans and animals.

As used herein, “prophylaxis” includes a use of the compound that, in a statistical sample, reduces the occurrence of the disorder or condition in the treated sample relative to an untreated control sample, or delays the onset or reduces the severity of one or more symptoms of the disorder or condition relative to the untreated control sample, when administered to prior to the onset of the disorder or condition.

Compounds of the present invention can be utilized to inhibit, block, reduce, decrease, etc., cell proliferation and/or cell division, and/or produce apoptosis, which are all types of “treatment”. This method comprises administering to a mammal in need thereof, including a human, an amount of a compound of general formula (I) of the present invention, or a pharmaceutically acceptable salt, isomer, polymorph, metabolite, hydrate, solvate or ester thereof, which is effective to treat the disorder.

Hyperproliferative disorders include, but are not limited to, for example: psoriasis, keloids, and other hyperplasias affecting the skin, benign prostate hyperplasia (BPH), solid tumours, such as cancers of the breast, respiratory tract, brain, reproductive organs, digestive tract, urinary tract, eye, liver, skin, head and neck, thyroid, parathyroid and their distant metastases. Those disorders also include lymphomas, sarcomas, and leukaemias.

Examples of breast cancers include, but are not limited to, invasive ductal carcinoma, invasive lobular carcinoma, ductal carcinoma in situ, and lobular carcinoma in situ.

Examples of cancers of the respiratory tract include, but are not limited to, small-cell and non-small-cell lung carcinoma, as well as bronchial adenoma and pleuropulmonary blastoma.

Examples of brain cancers include, but are not limited to, brain stem and hypophtalamic glioma, cerebellar and cerebral astrocytoma, medulloblastoma, ependymoma, as well as neuroectodermal and pineal tumour.

Tumours of the male reproductive organs include, but are not limited to, prostate and testicular cancer.

Tumours of the female reproductive organs include, but are not limited to, endometrial, cervical, ovarian, vaginal, and vulvar cancer, as well as sarcoma of the uterus.

Tumours of the digestive tract include, but are not limited to, anal, colon, colorectal, oesophageal, gallbladder, gastric, pancreatic, rectal, small-intestine, and salivary gland cancers.

Tumours of the urinary tract include, but are not limited to, bladder, penile, kidney, renal pelvis, ureter, urethral and human papillary renal cancers.

Eye cancers include, but are not limited to, intraocular melanoma and retinoblastoma.

Examples of liver cancers include, but are not limited to, hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to, basal cell carcinoma, squamous cell carcinoma, Kaposi's sarcoma, malignant melanoma, Merkel cell skin cancer and non-melanoma skin cancer.

Head-and-neck cancers include, but are not limited to, laryngeal, hypopharyngeal, nasopharyngeal, oropharyngeal cancer, lip and oral cavity cancer and squamous cell.

Lymphomas include, but are not limited to, AIDS-related lymphoma, chronic lymphocytic lymphoma (CLL), non-Hodgkin's lymphoma (NHL), T-non-Hodgkin lymphoma (T-NHL), subtypes of NHL such as Diffuse Large Cell Lymphoma (DLBCL), activated B-cell DLBCL, germinal center B-cell lymphoma DLBCL, double-hit lymphoma and double-expressor lymphoma; anaplastic large cell lymphoma, B-cell lymphoma, cutaneous T-cell lymphoma, Burkitt's lymphoma, follicular lymphoma, hairy cell lymphoma, Hodgkin's disease, mantle cell lymphoma (MCL), lymphoma of the central nervous system, small lymphocytic lymphoma and chronic lymphocytic lymphoma and Sezary syndrome.

Sarcomas include, but are not limited to, sarcoma of the soft tissue, osteosarcoma, malignant fibrous histiocytoma, lymphosarcoma, and rhabdomyosarcoma.

Leukemias include, but are not limited to acute lymphoblastic leukemia, acute myeloid leukemia, (acute) T-cell leukemia, acute lymphoblastic leukemia, acute lymphocytic leukemia (ALL), acute monocytic leukemia (AML), acute promyelocytic leukemia (APL), bisphenotypic B myelomonocytic leukemia, chronic lymphocytic leukemia (CLL), chronic myelogenous leukemia, chronic myeloid leukemia (CML), chronic myelomonocytic leukemia (CMML), large granular lymphocytic leukemia, plasma cell leukemia and also myelodysplastic syndrome (MDS), which can develop into an acute myeloid leukemia.

The present invention also provides methods of treating angiogenic disorders including diseases associated with excessive and/or abnormal angiogenesis.

Inappropriate and ectopic expression of angiogenesis can be deleterious to an organism. A number of pathological conditions are associated with the growth of extraneous blood vessels. These include, for example, diabetic retinopathy, ischemic retinal-vein occlusion, and retinopathy of prematurity [Aiello et al., New Engl. J. Med., 1994, 331, 1480; Peer et al., Lab. Invest., 1995, 72, 638], age-related macular degeneration (AMD) [Lopez et al., Invest. Ophthalmol. Vis. Sci., 1996, 37, 855], neovascular glaucoma, psoriasis, retrolental fibroplasias, angiofibroma, inflammation, rheumatoid arthritis (RA), restenosis, in-stent restenosis, vascular graft restenosis, etc. In addition, the increased blood supply associated with cancerous and neoplastic tissue, encourages growth, leading to rapid tumour enlargement and metastasis. Moreover, the growth of new blood and lymph vessels in a tumour provides an escape route for renegade cells, encouraging metastasis and the consequence spread of the cancer. Thus, compounds of general formula (I) of the present invention can be utilized to treat and/or prevent any of the aforementioned angiogenesis disorders, for example by inhibiting and/or reducing blood vessel formation; by inhibiting, blocking, reducing, decreasing, etc. endothelial cell proliferation, or other types involved in angiogenesis, as well as causing cell death or apoptosis of such cell types.

These disorders have been well characterized in humans, but also exist with a similar etiology in other mammals, and can be treated by administering pharmaceutical compositions of the present invention.

The term “treating” or “treatment” as stated throughout this document is used conventionally, for example the management or care of a subject for the purpose of combating, alleviating, reducing, relieving and/or improving the condition of a disease or disorder, such as a carcinoma.

The compounds of the present invention can be used in particular in therapy and prevention, i.e. prophylaxis, of tumour growth and metastases, especially in solid tumours of all indications and stages with or without pre-treatment of the tumour growth.

Generally, the use of chemotherapeutic agents and/or anti-cancer agents in combination with a compound or pharmaceutical composition of the present invention will serve to:
1. yield better efficacy in reducing the growth of a tumour or even eliminate the tumour as compared to administration of either agent alone,
2. provide for the administration of lesser amounts of the administered chemotherapeutic agents,
3. provide for a chemotherapeutic treatment that is well tolerated in the patient with fewer deleterious pharmacological complications than observed with single agent chemotherapies and certain other combined therapies,
4. provide for treating a broader spectrum of different cancer types in mammals, especially humans,
5. provide for a higher response rate among treated patients,
6. provide for a longer survival time among treated patients compared to standard chemotherapy treatments,
7. provide a longer time for tumour progression, and/or
8. yield efficacy and tolerability results at least as good as those of the agents used alone, compared to known instances where other cancer agent combinations produce antagonistic effects.

In addition, the compounds of general formula (I) of the present invention can also be used in combination with radiotherapy and/or surgical intervention.

In a further embodiment of the present invention, the compounds of general formula (I) of the present invention may be used to sensitize a cell to radiation, i.e. treatment of a cell with a compound of the present invention prior to radiation treatment of the cell renders the cell more susceptible to DNA damage and cell death than the cell would be in the absence of any treatment with a compound of the present invention. In one aspect, the cell is treated with at least one compound of general formula (I) of the present invention.

Thus, the present invention also provides a method of killing a cell, wherein a cell is administered one or more compounds of the present invention in combination with conventional radiation therapy.

The present invention also provides a method of rendering a cell more susceptible to cell death, wherein the cell is treated with one or more compounds of general formula (I) of the present invention prior to the treatment of the cell to cause or induce cell death. In one aspect, after the cell is treated with one or more compounds of general formula (I) of the present invention, the cell is treated with at least one compound, or at least one method, or a combination thereof, in order to cause DNA damage for the purpose of inhibiting the function of the cell or killing the cell.

In other embodiments of the present invention, a cell is killed by treating the cell with at least one DNA damaging agent, i.e. after treating a cell with one or more compounds of general formula (I) of the present invention to sensitize the cell to cell death, the cell is treated with at least one DNA damaging agent to kill the cell. DNA damaging agents useful in the present invention include, but are not limited to, chemotherapeutic agents (e.g. cis platin), ionizing radiation (X-rays, ultraviolet radiation), carcinogenic agents, and mutagenic agents.

In other embodiments, a cell is killed by treating the cell with at least one method to cause or induce DNA damage. Such methods include, but are not limited to, activation of a cell signalling pathway that results in DNA damage when the pathway is activated, inhibiting of a cell signalling pathway that results in DNA damage when the pathway is inhibited, and inducing a biochemical change in a cell, wherein the change results in DNA damage.

By way of a non-limiting example, a DNA repair pathway in a cell can be inhibited, thereby preventing the repair of DNA damage and resulting in an abnormal accumulation of DNA damage in a cell.

In some embodiments, a compound of general formula (I) of the present invention is administered to a cell prior to the radiation or other induction of DNA damage in the cell.

In some embodiments of the invention, a compound of general formula (I) of the present invention is administered to a cell concomitantly with the radiation or other induction of DNA damage in the cell. In yet some embodiments of the invention, a compound of general formula (I) of the present invention is administered to a cell after radiation or other induction of DNA damage in the cell has begun. In yet some embodiments of the invention, a compound of general formula (I) of the present invention is administered to a cell immediately after radiation or other induction of DNA damage in the cell has begun.

In some embodiments, the cell is in vitro. In another embodiment, the cell is in vivo.

Thus in some embodiments, the present invention includes a method of inhibiting proliferation of a cell and/or the induction of apoptosis in a cell, comprising contacting the cell with a compound of formula (I).

Another aspect of the invention is a method for treating, preventing or prophylaxing cancer (i.e. a method for the treatment, prevention or prophylaxis of cancer) in a subject (e.g., human, other mammal, such as rat, etc.) by administering an effective amount of at least one compound of general formula (I), or a pharmaceutically acceptable salt, polymorph, metabolite, hydrate, solvate or ester thereof to the subject.

In some embodiments, the subject may be administered a medicament, comprising at least one compound of general formula (I) and one or more pharmaceutically acceptable carriers, excipients and/or diluents.

Furthermore in some embodiments, the present invention includes a method of using a compound of general formula (I) for the treatment of diseases.

Particularly in some embodiments, the present invention includes a method of treating a hyperproliferative disease, more particularly cancer, comprising administering an effective amount of at least one compound of general formula (I) to a subject in need thereof.

In some embodiments, the method of treatment and/or prophylaxis of a hyperproliferative disorder in a subject may comprise administering to the subject an effective amount of a compound of general formula (I). The hyperproliferative disorder may be, for example, cancer (e.g., lung cancer, acute myeloid leukemia, lymphoma, glioblastoma, prostate cancer, etc.).

Furthermore in some embodiments, the present invention includes a method of treating cancer, particularly lymphoma, non-Hodgkin-lymphoma type, diffuse large B-cell lymphoma subtype, acute leukemia, acute myeloid leukemia type, multiple myeloma, ovarian cancer, comprising administering an effective amount of at least one compound of formula (I) to a subject in need thereof.

Furthermore in some embodiments, the present invention includes a method of treating cancer, particularly multiple myeloma, ovarian carcinoma, acute monocytic leukemia, melanoma and lung cancer, comprising administering an effective amount of at least one compound of formula (I) to a subject in need thereof.

Furthermore in some embodiments, the present invention includes a method of treating cancer, particularly breast cancer; lung cancer; lymphoma including non-Hodgkin-lymphoma type, diffuse large B-cell lymphoma subtype including GC-DLBCL*and ABC-DLBCL** subtypes, and mantle cell lymphoma; acute leukemia, acute myeloid leukemia type, acute monocytic leukemia; melanoma; multiple myeloma; ovarian cancer; and pancreas cancer, comprising administering an effective amount of at least one compound of formula (I) to a subject in need thereof. GC-DLBCL means Germinal B-cell Diffuse Large B-Cell Lymphoma and ** ABC-DLBCL means Activated B-cell Diffuse Large B-Cell Lymphoma.

Furthermore in some embodiments, the present invention includes a method of treating cancer, particularly breast cancer, lung cancer, diffuse large B-cell lymphoma subtype including GC-DLBCL*and ABC-DLBCL** subtypes, mantle cell lymphoma, acute monocytic leukemia, melanoma, ovarian cancer, and pancreas cancer comprising administering an effective amount of at least one compound of formula (I) to a subject in need thereof. Furthermore in some embodiments, the present invention provides a compound of formula (I) for use of treating diseases.

Furthermore in some embodiments, the present invention includes a method of treating cancer, particularly breast cancer; lymphoma, leukemia, multiple myeloma; and ovarian cancer, comprising administering an effective amount of at least one compound of formula (I) to a subject in need thereof.

Furthermore in some embodiments, the present invention includes a method of treating cancer, particularly lymphoma, non-Hodgkin-lymphoma type, diffuse large B-cell lymphoma subtype, acute leukemia, acute myeloid leukemia type, multiple myeloma, and ovarian cancer, comprising administering an effective amount of at least one compound of formula (I) to a subject in need thereof.

Furthermore in some embodiments, the present invention includes a method of treating cancer, particularly breast cancer, lymphoma (including non-Hodgkin-lymphoma type, diffuse large B-cell lymphoma subtype, mantle cell lymphoma), leukemia (including acute monocytic leukemia and acute myeloid leukemia), liver cancer, multiple myeloma, melanoma, non-small cell lung cancer, small cell lung cancer, ovarian cancer, ovarian carcinoma, stomach cancer, and squamous cell carcinoma, comprising administering an effective amount of at least one compound of formula (I) to a subject in need thereof.

Furthermore in some embodiments, the present invention includes a method of treating cancer, particularly breast cancer, diffuse large B-cell lymphoma subtype, mantle cell lymphoma, acute monocytic leukemia, acute myeloid leukemia, liver cancer, multiple myeloma, melanoma, non-small cell lung cancer, small cell lung cancer, ovarian cancer, ovarian carcinoma, prostate cancer, stomach cancer, and squamous cell carcinoma, comprising administering an effective amount of at least one compound of formula (I) to a subject in need thereof.

Furthermore in some embodiments, the present invention includes a method of treating cancer, particularly bladder cancer, bone cancer, brain cancer, breast cancer, colon cancer (colorectal cancer), endometrial (uterine) cancer, gastric cancer, head and neck cancer, kidney cancer, leukemia, liver cancer, lung cancer, lymphoma, lung cancer, myeloma, neuroblastoma, ovarian cancer, pancreatic cancer, rhabdoid tumor, sarcoma and skin cancer, comprising administering an effective amount of at least one compound of formula (I) to a subject in need thereof.

Furthermore in some embodiments, the present invention includes a method of treating cancer, particularly lung cancer, breast cancer, bladder cancer, uterine cancer, endometrial cancer, prostate cancer and leukemia comprising administering an effective amount of at least one compound of formula (I) to a subject in need thereof.

Furthermore in some embodiments, the present invention includes a method of treating cancer, particularly cancer in which KAT6A and/or KAT6B is focally amplified, said method comprising administering an effective amount of at least one compound of formula (I) to a subject in need thereof.

Furthermore in some embodiments, the present invention includes a method of treating cancer, particularly cancer in which KAT6A is focally amplified, said method comprising administering an effective amount of at least one compound of formula (I) to a subject in need thereof.

Furthermore in some embodiments, the present invention includes a method of treating cancer, particularly cancer in which KAT6B is focally amplified, said method comprising administering an effective amount of at least one compound of formula (I) to a subject in need thereof.

Furthermore in some embodiments, the present invention includes a method of treating cancer, particularly breast cancer, lung cancer, endometrial cancer, ovarian cancer, bladder cancer, esophageal cancer, uterine cancer and epithelial cancer comprising administering an effective amount of at least one compound of formula (I) to a subject in need thereof.

Furthermore in some embodiments, the present invention includes a method of treating cancer, particularly breast cancer, lung cancer, endometrial cancer, ovarian cancer, bladder cancer and esophageal cancer comprising administering an effective amount of at least one compound of formula (I) to a subject in need thereof.

Furthermore in some embodiments, the present invention includes a method of treating cancer, particularly breast cancer, uterine cancer and epithelial cancer comprising administering an effective amount of at least one compound of formula (I) to a subject in need thereof.

In accordance with some embodiments, the present invention provides compounds of general formula (I), as described supra, or stereoisomers, tautomers, N-oxides, hydrates, solvates, and salts thereof, particularly pharmaceutically acceptable salts thereof, or mixtures of same, for use in the treatment or prophylaxis of diseases, in particular hyperproliferative disorders.

Furthermore in accordance with a further aspect, the present invention provides a compound of formula (I) for use of treating diseases.

In accordance with a further aspect, the present invention includes a compound of general formula (I) for use in a method of inhibiting proliferation of a cell and/or the induction of apoptosis in a cell, comprising contacting the cell with a compound of formula (I).

Particularly in some embodiments, the present invention includes compounds of general formula (I) for use in a method of treating a hyperproliferative disease, more particularly wherein the hyperproliferative disease is cancer, and yet even more particularly wherein the cancer disease is selected from lymphoma, non-Hodgkin-lymphoma type, diffuse large B-cell lymphoma subtype, ovarian cancer, multiple myeloma, acute leukemia, and acute myeloid leukemia.

More particularly in some embodiments, the present invention includes compounds of general formula (I) for use in a method of treating a hyperproliferative disease, more particularly wherein the hyperproliferative disease is cancer, and yet even more particularly wherein the cancer disease is selected from breast cancer; lymphoma, leukemia, multiple myeloma; and ovarian cancer.

Particularly in some embodiments, the present invention includes compounds of general formula (I) for use in a method of treating a hyperproliferative disease, more particularly wherein the hyperproliferative disease is cancer, and yet even more particularly wherein the cancer is selected from breast cancer; esophageal cancer; liver cancer; lung cancer; lymphoma including non-Hodgkin-lymphoma type, diffuse large B-cell lymphoma subtype including GC-DLBCL*and ABC-DLBCL** subtypes, and mantle cell lymphoma; acute leukemia, acute myeloid leukemia type, acute monocytic leukemia; melanoma; multiple myeloma; melanoma; ovarian cancer; or pancreas cancer.

More particularly in some embodiments, the present invention includes compounds of general formula (I) for use in a method of treating cancer wherein the cancer disease is selected from breast cancer; lymphoma, leukemia, multiple myeloma; and ovarian cancer.

More particularly in some embodiments, the present invention includes compounds of general formula (I) for use in a method of treating cancer wherein the cancer disease is selected from lung cancer, breast cancer, bladder cancer, uterine cancer, endometrial cancer, prostate cancer and leukemia.

More particularly in some embodiments, the present invention includes compounds of general formula (I) for use in a method of treating cancer in which KAT6A and/or KAT6B is focally amplified.

More particularly in some embodiments, the present invention includes compounds of general formula (I) for use in a method of treating cancer in which KAT6A is focally amplified.

More particularly in some embodiments, the present invention includes compounds of general formula (I) for use in a method of treating cancer in which KAT6B is focally amplified.

More particularly in some embodiments, the present invention includes compounds of general formula (I) for use in a method of treating cancer wherein the cancer disease is selected from breast cancer, lung cancer, endometrial cancer, ovarian cancer, bladder cancer, esophageal cancer, uterine cancer and epithelial cancer.

More particularly in some embodiments, the present invention includes compounds of general formula (I) for use in a method of treating cancer wherein the cancer disease is selected from breast cancer, lung cancer, endometrial cancer, ovarian cancer, bladder cancer and esophageal cancer.

More particularly in some embodiments, the present invention includes compounds of general formula (I) for use in a method of treating cancer wherein the cancer disease is selected from breast cancer, uterine cancer and epithelial cancer.

In some embodiments, the present invention includes the use of the compounds of general formula (I) for the manufacture of a medicament for the treatment and/or prophylaxis of a hyperproliferative disease.

In some embodiments, the present invention includes the use of the compounds of general formula (I) for the manufacture of a medicament for the treatment and/or prophylaxis of a hyperproliferative disease, wherein the hyperproliferative disease is cancer.

In some embodiments, the present invention includes the use of the compounds of general formula (I) for the manufacture of a medicament for the treatment of a hyperproliferative disease, particularly cancer and more particularly lymphoma, non-Hodgkin-lymphoma type, diffuse large B-cell lymphoma subtype, ovarian cancer, multiple myeloma, acute leukemia, and acute myeloid leukemia type.

In some embodiments, the present invention includes the use of the compounds of general formula (I) for the manufacture of a medicament for the treatment of a hyperproliferative disease, particularly cancer and more particularly lung cancer, breast cancer, bladder cancer, uterine cancer, endometrial cancer, prostate cancer and leukemia.

In some embodiments, the present invention includes the use of the compounds of general formula (I) for the manufacture of a medicament for the treatment of a hyperproliferative disease, particularly cancer and more particularly cancer in which KAT6A and/or KAT6B is focally amplified.

In some embodiments, the present invention includes the use of the compounds of general formula (I) for the manufacture of a medicament for the treatment of a hyperproliferative disease, particularly cancer and more particularly cancer in which KAT6A is focally amplified.

In some embodiments, the present invention includes the use of the compounds of general formula (I) for the manufacture of a medicament for the treatment of a hyperproliferative disease, particularly cancer and more particularly cancer in which KAT6B is focally amplified.

In some embodiments, the present invention includes the use of the compounds of general formula (I) for the manufacture of a medicament for the treatment of a hyperproliferative disease, particularly cancer and more particularly breast cancer, lung cancer, endometrial cancer, ovarian cancer, bladder cancer, esophageal cancer, uterine cancer and epithelial cancer.

In some embodiments, the present invention includes the use of the compounds of general formula (I) for the manufacture of a medicament for the treatment of a hyperproliferative disease, particularly cancer and more particularly breast cancer, lung cancer, endometrial cancer, ovarian cancer, bladder cancer and esophageal cancer.

In some embodiments, the present invention includes the use of the compounds of general formula (I) for the manufacture of a medicament for the treatment of a hyperproliferative disease, particularly cancer and more particularly breast cancer, uterine cancer and epithelial cancer.

In some embodiments, the present invention provides use of a compound of general formula (I), as described supra, or stereoisomers, tautomers, N-oxides, hydrates, solvates, and salts thereof, particularly pharmaceutically acceptable salts thereof, or mixtures of same, for the preparation of a pharmaceutical composition, preferably a medicament, for the prophylaxis or treatment of diseases, in particular hyperproliferative disorders, particularly cancer.

In some embodiments, the present invention provides use of a compound of general formula (I), as described supra, or stereoisomers, tautomers, N-oxides, hydrates, solvates, and salts thereof, particularly pharmaceutically acceptable salts thereof, or mixtures of same, for the preparation of a pharmaceutical composition, preferably a medicament, for the prophylaxis or treatment of diseases, in particular hyperproliferative disorders, particularly cancer, more particularly lung cancer, breast cancer, bladder cancer, uterine cancer, endometrial cancer, prostate cancer and leukemia.

In some embodiments, the present invention provides use of a compound of general formula (I), as described supra, or stereoisomers, tautomers, N-oxides, hydrates, solvates, and salts thereof, particularly pharmaceutically acceptable salts thereof, or mixtures of same, for the preparation of a pharmaceutical composition, preferably a medicament, for the prophylaxis or treatment of diseases, in particular hyperproliferative disorders, particularly cancer, more particularly cancer in which KAT6A and/or KAT6B is focally amplified.

In some embodiments, the present invention provides use of a compound of general formula (I), as described supra, or stereoisomers, tautomers, N-oxides, hydrates, solvates, and salts thereof, particularly pharmaceutically acceptable salts thereof, or mixtures of same, for the preparation of a pharmaceutical composition, preferably a medicament, for the prophylaxis or treatment of diseases, in particular hyperproliferative disorders, particularly cancer, more particularly cancer in which KAT6A is focally amplified.

In some embodiments, the present invention provides use of a compound of general formula (I), as described supra, or stereoisomers, tautomers, N-oxides, hydrates, solvates, and salts thereof, particularly pharmaceutically acceptable salts thereof, or mixtures of same, for the preparation of a pharmaceutical composition, preferably a medicament, for the prophylaxis or treatment of diseases, in particular hyperproliferative disorders, particularly cancer, more particularly cancer in which KAT6B is focally amplified.

In some embodiments, the present invention provides use of a compound of general formula (I), as described supra, or stereoisomers, tautomers, N-oxides, hydrates, solvates, and salts thereof, particularly pharmaceutically acceptable salts thereof, or mixtures of same, for the preparation of a pharmaceutical composition, preferably a medicament, for the prophylaxis or treatment of diseases, in particular hyperproliferative disorders, particularly cancer, more particularly breast cancer, lung cancer, endometrial cancer, ovarian cancer, bladder cancer, esophageal cancer, uterine cancer and epithelial cancer.

In some embodiments, the present invention provides use of a compound of general formula (I), as described supra, or stereoisomers, tautomers, N-oxides, hydrates, solvates, and salts thereof, particularly pharmaceutically acceptable salts thereof, or mixtures of same, for the preparation of a pharmaceutical composition, preferably a medicament, for the prophylaxis or treatment of diseases, in particular hyperproliferative disorders, particularly cancer, more particularly breast cancer, lung cancer, endometrial cancer, ovarian cancer, bladder cancer and esophageal cancer.

In some embodiments, the present invention provides use of a compound of general formula (I), as described supra, or stereoisomers, tautomers, N-oxides, hydrates, solvates, and salts thereof, particularly pharmaceutically acceptable salts thereof, or mixtures of same, for the preparation of a pharmaceutical composition, preferably a medicament, for the prophylaxis or treatment of diseases, in particular hyperproliferative disorders, particularly cancer, more particularly breast cancer, uterine cancer and epithelial cancer.

In some embodiments, the present invention provides a method of treatment or prophylaxis of diseases, in particular hyperproliferative disorders, particularly cancer, comprising administering an effective amount of a compound of general formula (I), as described supra, or stereoisomers, tautomers, N-oxides, hydrates, solvates, and salts thereof, particularly pharmaceutically acceptable salts thereof, or mixtures of same to a subject in need thereof.

In some embodiments, the present invention provides a method of treatment or prophylaxis of diseases, in particular hyperproliferative disorders, particularly cancer, more particularly lung cancer, breast cancer, bladder cancer, uterine cancer, endometrial cancer, prostate cancer and leukemia comprising administering an effective amount of a compound of general formula (I), as described supra, or stereoisomers, tautomers, N-oxides, hydrates, solvates, and salts thereof, particularly pharmaceutically acceptable salts thereof, or mixtures of same to a subject in need thereof.

In some embodiments, the present invention provides a method of treatment or prophylaxis of diseases, in particular hyperproliferative disorders, particularly cancer, more particularly cancer in which KAT6A and/or KAT6B is focally amplified comprising administering an effective amount of a compound of general formula (I), as described supra, or stereoisomers, tautomers, N-oxides, hydrates, solvates, and salts thereof, particularly pharmaceutically acceptable salts thereof, or mixtures of same to a subject in need thereof.

In some embodiments, the present invention provides a method of treatment or prophylaxis of diseases, in particular hyperproliferative disorders, particularly cancer, more particularly cancer in which KAT6A is focally amplified comprising administering an effective amount of a compound of general formula (I), as described supra, or stereoisomers, tautomers, N-oxides, hydrates, solvates, and salts thereof, particularly pharmaceutically acceptable salts thereof, or mixtures of same to a subject in need thereof.

In some embodiments, the present invention provides a method of treatment or prophylaxis of diseases, in particular hyperproliferative disorders, particularly cancer, more particularly cancer in which KAT6B is focally amplified comprising administering an effective amount of a compound of general formula (I), as described supra, or stereoisomers, tautomers, N-oxides, hydrates, solvates, and salts thereof, particularly pharmaceutically acceptable salts thereof, or mixtures of same to a subject in need thereof.

In some embodiments, the present invention provides a method of treatment or prophylaxis of diseases, in particular hyperproliferative disorders, particularly cancer, more particularly breast cancer, lung cancer, endometrial cancer, ovarian cancer, bladder cancer, esophageal cancer, uterine cancer and epithelial cancer comprising administering an effective amount of a compound of general formula (I), as described supra, or stereoisomers, tautomers, N-oxides, hydrates, solvates, and salts thereof, particularly pharmaceutically acceptable salts thereof, or mixtures of same to a subject in need thereof.

In some embodiments, the present invention provides a method of treatment or prophylaxis of diseases, in particular hyperproliferative disorders, particularly cancer, more particularly breast cancer, lung cancer, endometrial cancer, ovarian cancer, bladder cancer and esophageal cancer comprising administering an effective amount of a compound of general formula (I), as described supra, or stereoisomers, tautomers, N-oxides, hydrates, solvates, and salts thereof, particularly pharmaceutically acceptable salts thereof, or mixtures of same to a subject in need thereof.

In some embodiments, the present invention provides a method of treatment or prophylaxis of diseases, in particular hyperproliferative disorders, particularly cancer, more particularly breast cancer, uterine cancer and epithelial cancer comprising administering an effective amount of a compound of general formula (I), as described supra, or stereoisomers, tautomers, N-oxides, hydrates, solvates, and salts thereof, particularly pharmaceutically acceptable salts thereof, or mixtures of same to a subject in need thereof.

In some embodiments, the present invention provides pharmaceutical compositions, in particular a medicament, comprising a compound of general formula (I), as described supra, or a stereoisomer, a tautomer, an N-oxide, a hydrate, a solvate, a salt thereof, particularly a pharmaceutically acceptable salt, or a mixture of same, and one or more excipients), in particular one or more pharmaceutically acceptable excipient(s). Conventional procedures for preparing such pharmaceutical compositions in appropriate dosage forms can be utilized.

The present invention furthermore provides pharmaceutical compositions, in particular medicaments, which comprise at least one compound according to the invention, conventionally together with one or more pharmaceutically suitable excipients, and to their use for the above mentioned purposes.

It is possible for the compounds according to the invention to have systemic and/or local activity. For this purpose, they can be administered in a suitable manner, such as, for example, via the oral, parenteral, pulmonary, nasal, sublingual, lingual, buccal, rectal, vaginal, dermal, transdermal, conjunctival, otic route or as an implant or stent.

For these administration routes, it is possible for the compounds according to the invention to be administered in suitable administration forms.

For oral administration, it is possible to formulate the compounds according to the invention to dosage forms known in the art that deliver the compounds of the invention rapidly and/or in a modified manner, such as, for example, tablets (uncoated or coated tablets, for example with enteric or controlled release coatings that dissolve with a delay or are insoluble), orally-disintegrating tablets, films/wafers, films/lyophylisates, capsules (for example hard or soft gelatine capsules), sugar-coated tablets, granules, pellets, powders, emulsions, suspensions, aerosols or solutions. It is possible to incorporate the compounds according to the invention in crystalline and/or amorphised and/or dissolved form into said dosage forms.

Parenteral administration can be effected with avoidance of an absorption step (for example intravenous, intraarterial, intracardial, intraspinal or intralumbal) or with inclusion of absorption (for example intramuscular, subcutaneous, intracutaneous, percutaneous or intraperitoneal). Administration forms which are suitable for parenteral administration are, inter alia, preparations for injection and infusion in the form of solutions, suspensions, emulsions, lyophylisates or sterile powders.

Examples which are suitable for other administration routes are pharmaceutical forms for inhalation [inter alia powder inhalers, nebulizers], nasal drops, nasal solutions, nasal sprays; tablets/films/wafers/capsules for lingual, sublingual or buccal administration; suppositories; eye drops, eye ointments, eye baths, ocular inserts, ear drops, ear sprays, ear powders, ear-rinses, ear tampons; vaginal capsules, aqueous suspensions (lotions, mixture agitandae), lipophilic suspensions, emulsions, ointments, creams, transdermal therapeutic systems (such as, for example, patches), milk, pastes, foams, dusting powders, implants or stents.

The compounds according to the invention can be incorporated into the stated administration forms. This can be effected in a manner known per se by mixing with pharmaceutically suitable excipients. Pharmaceutically suitable excipients include, inter alia,
fillers and carriers (for example cellulose, microcrystalline cellulose (such as, for example, Avicel®), lactose, mannitol, starch, calcium phosphate (such as, for example, Di-Cafos®)),
ointment bases (for example petroleum jelly, paraffins, triglycerides, waxes, wool wax, wool wax alcohols, lanolin, hydrophilic ointment, polyethylene glycols),
bases for suppositories (for example polyethylene glycols, cacao butter, hard fat),
solvents (for example water, ethanol, isopropanol, glycerol, propylene glycol, medium chain-length triglycerides fatty oils, liquid polyethylene glycols, paraffins),
surfactants, emulsifiers, dispersants or wetters (for example sodium dodecyl sulfate), lecithin, phospholipids, fatty alcohols (such as, for example, Lanette®), sorbitan fatty acid esters (such as, for example, Span®), polyoxyethylene sorbitan fatty acid esters (such as, for example, Tween®), polyoxyethylene fatty acid glycerides (such as, for example, Cremophor®), polyoxethylene fatty acid esters, polyoxyethylene fatty alcohol ethers, glycerol fatty acid esters, poloxamers (such as, for example, Pluronic®),
buffers, acids and bases (for example phosphates, carbonates, citric acid, acetic acid, hydrochloric acid, sodium hydroxide solution, ammonium carbonate, trometamol, triethanolamine),
isotonicity agents (for example glucose, sodium chloride), “adsorbents (for example highly-disperse silicas),
viscosity-increasing agents, gel formers, thickeners and/or binders (for example polyvinylpyrrolidone, methylcellulose, hydroxypropylmethylcellulose, hydroxypropylcellulose, carboxymethylcellulose-sodium, starch, carbomers, polyacrylic acids (such as, for example, Carbopol); alginates, gelatine),
disintegrants (for example modified starch, carboxymethylcellulose-sodium, sodium starch glycolate (such as, for example, Explotab®), cross- linked polyvinylpyrrolidone, croscarmellose-sodium (such as, for example, AcDiSol®)),
flow regulators, lubricants, glidants and mould release agents (for example magnesium stearate, stearic acid, talc, highly-disperse silicas (such as, for example, Aerosil®)),
coating materials (for example sugar, shellac) and film formers for films or diffusion membranes which dissolve rapidly or in a modified manner (for example polyvinylpyrrolidones (such as, for example, Kollidon®), polyvinyl alcohol, hydroxypropylmethylcellulose, hydroxypropylcellulose, ethylcellulose, hydroxypropylmethylcellulose phthalate, cellulose acetate, cellulose acetate phthalate, polyacrylates, polymethacrylates such as, for example, Eudragit®)),
capsule materials (for example gelatine, hydroxypropylmethylcellulose),
synthetic polymers (for example polylactides, polyglycolides, polyacrylates, polymethacrylates (such as, for example, Eudragit®), polyvinylpyrrolidones (such as, for example, Kollidon®), polyvinyl alcohols, polyvinyl acetates, polyethylene oxides, polyethylene glycols and their copolymers and blockcopolymers),
plasticizers (for example polyethylene glycols, propylene glycol, glycerol, triacetine, triacetyl citrate, dibutyl phthalate),
penetration enhancers,
stabilisers (for example antioxidants such as, for example, ascorbic acid, ascorbyl palmitate, sodium ascorbate, butylhydroxyanisole, butylhydroxytoluene, propyl gallate),
preservatives (for example parabens, sorbic acid, thiomersal, benzalkonium chloride, chlorhexidine acetate, sodium benzoate),
colourants (for example inorganic pigments such as, for example, iron oxides, titanium dioxide),
flavourings, sweeteners, flavour- and/or odour-masking agents.

The present invention furthermore relates to a pharmaceutical composition which comprise at least one compound according to the invention, conventionally together with one or more pharmaceutically suitable excipient(s), and to their use according to the present invention.

In some embodiments, the present invention provides pharmaceutical combinations, in particular medicaments, comprising at least one compound of general formula (I) of the present invention and at least one or more further active ingredients, in particular for the treatment and/or prophylaxis of a hyperproliferative disorder, particularly cancer.

Particularly, the present invention provides a pharmaceutical combination, which comprises:
one or more first active ingredients, in particular compounds of general formula (I) as defined supra, and
one or more further active ingredients, in particular for the treatment and/or prophylaxis of a hyperproliferative disorder, particularly cancer.

The term “combination” in the present invention is used as known to persons skilled in the art, it being possible for said combination to be a fixed combination, a non-fixed combination or a kit-of-parts.

A “fixed combination” in the present invention is used as known to persons skilled in the art and is defined as a combination wherein, for example, a first active ingredient, such as one or more compounds of general formula (I) of the present invention, and a further active ingredient are present together in one unit dosage or in one single entity. One example of a “fixed combination” is a pharmaceutical composition wherein a first active ingredient and a further active ingredient are present in admixture for simultaneous administration, such as in a formulation. Another example of a “fixed combination” is a pharmaceutical combination wherein a first active ingredient and a further active ingredient are present in one unit without being in admixture.

A non-fixed combination or “kit-of-parts” in the present invention is used as known to persons skilled in the art and is defined as a combination wherein a first active ingredient and a further active ingredient are present in more than one unit. One example of a non-fixed combination or kit-of-parts is a combination wherein the first active ingredient and the further active ingredient are present separately. It is possible for the components of the non-fixed combination or kit-of-parts to be administered separately, sequentially, simultaneously, concurrently or chronologically staggered.

The compounds of the present invention can be administered as the sole pharmaceutical agent or in combination with one or more other pharmaceutically active ingredients where the combination causes no unacceptable adverse effects. The present invention also provides such pharmaceutical combinations. For example, the compounds of the present invention can be combined with known anti-cancer agents.

Examples of anti-cancer agents include:

131I-chTNT, abarelix, abemaciclib, abiraterone, acalabrutinib, aclarubicin, adalimumab, ado-trastuzumab emtansine, afatinib, aflibercept, aldesleukin, alectinib, alemtuzumab, alendronic acid, alitretinoin, altretamine, amifostine, aminoglutethimide, hexyl aminolevulinate, amrubicin, amsacrine, anastrozole, ancestim, anethole dithiolethione, anetumab ravtansine, angiotensin II, antithrombin III, apalutamide, aprepitant, arcitumomab, arglabin, arsenic trioxide, asparaginase, atezolizumab, avelumab, axicabtagene ciloleucel, axitinib, azacitidine, basiliximab, belotecan, bendamustine, besilesomab, belinostat, bevacizumab, bexarotene, bicalutamide, bisantrene, bleomycin, blinatumomab, bortezomib, bosutinib, buserelin, brentuximab vedotin, brigatinib, busulfan, cabazitaxel, cabozantinib, calcitonine, calcium folinate, calcium levofolinate, capecitabine, capromab, carbamazepine carboplatin, carboquone, carfilzomib, carmofur, carmustine, catumaxomab, celecoxib, celmoleukin, ceritinib, cetuximab, chlorambucil, chlormadinone, chlormethine, cidofovir, cinacalcet, cisplatin, cladribine, clodronic acid, clofarabine, cobimetinib, copanlisib, crisantaspase, crizotinib, cyclophosphamide, cyproterone, cytarabine, dacarbazine, dactinomycin, daratumumab, darbepoetin alfa, dabrafenib, dasatinib, daunorubicin, decitabine, degarelix, denileukin diftitox, denosumab, depreotide, deslorelin, dianhydrogalactitol, dexrazoxane, dibrospidium chloride, dianhydrogalactitol, diclofenac, dinutuximab, docetaxel, dolasetron, doxifluridine, doxorubicin, doxorubicin+estrone, dronabinol, durvalumab, eculizumab, edrecolomab, elliptinium acetate, elotuzumab, eltrombopag, enasidenib, endostatin, enocitabine, enzalutamide, epirubicin, epitiostanol, epoetin alfa, epoetin beta, epoetin zeta, eptaplatin, eribulin, erlotinib, esomeprazole, estradiol, estramustine, ethinylestradiol, etoposide, everolimus, exemestane, fadrozole, fentanyl, filgrastim, fluoxymesterone, floxuridine, fludarabine, fluorouracil, flutamide, folinic acid, formestane, fosaprepitant, fotemustine, fulvestrant, gadobutrol, gadoteridol, gadoteric acid meglumine, gadoversetamide, gadoxetic acid, gallium nitrate, ganirelix, gefitinib, gemcitabine, gemtuzumab, Glucarpidase, glutoxim, GM-CSF, goserelin, granisetron, granulocyte colony stimulating factor, histamine dihydrochloride, histrelin, hydroxycarbamide, I-125 seeds, lansoprazole, ibandronic acid, ibritumomab tiuxetan, ibrutinib, idarubicin, ifosfamide, imatinib, imiquimod, improsulfan, indisetron, incadronic acid, ingenol mebutate, inotuzumab ozogamicin, interferon alfa, interferon beta, interferon gamma, iobitridol, iobenguane (123I), iomeprol, ipilimumab, irinotecan, Itraconazole, ixabepilone, ixazomib, lanreotide, lansoprazole, lapatinib, lasocholine, lenalidomide, lenvatinib, lenograstim, lentinan, letrozole, leuprorelin, levamisole, levonorgestrel, levothyroxine sodium, lisuride, lobaplatin, lomustine, lonidamine, lutetium Lu 177 dotatate, masoprocol, medroxyprogesterone, megestrol, melarsoprol, melphalan, mepitiostane, mercaptopurine, mesna, methadone, methotrexate, methoxsalen, methylaminolevulinate, methylprednisolone, methyltestosterone, metirosine, midostaurin, mifamurtide, miltefosine, miriplatin, mitobronitol, mitoguazone, mitolactol, mitomycin, mitotane, mitoxantrone, mogamulizumab, molgramostim, mopidamol, morphine hydrochloride, morphine sulfate, mvasi, nabilone, nabiximols, nafarelin, naloxone+pentazocine, naltrexone, nartograstim, necitumumab, nedaplatin, nelarabine, neratinib, neridronic acid, netupitant/palonosetron, nivolumab, pentetreotide, nilotinib, nilutamide, nimorazole, nimotuzumab, nimustine, nintedanib, niraparib, nitracrine, nivolumab, obinutuzumab, octreotide, ofatumumab, olaparib, olaratumab, omacetaxine mepesuccinate, omeprazole, ondansetron, oprelvekin, orgotein, orilotimod, osimertinib, oxaliplatin, oxycodone, oxymetholone, ozogamicine, p53 gene therapy, paclitaxel, palbociclib, palifermin, palladium-103 seed, palonosetron, pamidronic acid, panitumumab, panobinostat, pantoprazole, pazopanib, pegaspargase, PEG-epoetin beta (methoxy PEG-epoetin beta), pembrolizumab, pegfilgrastim, peginterferon alfa-2b, pembrolizumab, pemetrexed, pentazocine, pentostatin, peplomycin, Perflubutane, perfosfamide, Pertuzumab, picibanil, pilocarpine, pirarubicin, pixantrone, plerixafor, plicamycin, poliglusam, polyestradiol phosphate, polyvinylpyrrolidone+sodium hyaluronate, polysaccharide-K, pomalidomide, ponatinib, porfimer sodium, pralatrexate, prednimustine, prednisone, procarbazine, procodazole, propranolol, quinagolide, rabeprazole, racotumomab, radium-223 chloride, radotinib, raloxifene, raltitrexed, ramosetron, ramucirumab, ranimustine, rasburicase, razoxane, refametinib, regorafenib, ribociclib, risedronic acid, rhenium-186 etidronate, rituximab, rolapitant, romidepsin, romiplostim, romurtide, rucaparib, samarium (153Sm) lexidronam, sargramostim, sarilumab, satumomab, secretin, siltuximab, sipuleucel-T, sizofiran, sobuzoxane, sodium glycididazole, sonidegib, sorafenib, stanozolol, streptozocin, sunitinib, talaporfin, talimogene laherparepvec, tamibarotene, tamoxifen, tapentadol, tasonermin, teceleukin, technetium (99mTc) nofetumomab merpentan, 99mTc-HYNIC-[Tyr3]-octreotide, tegafur, tegafur+gimeracil+oteracil, temoporfin, temozolomide, temsirolimus, teniposide, testosterone, tetrofosmin, thalidomide, thiotepa, thymalfasin, thyrotropin alfa, tioguanine, tisagenlecleucel, tocilizumab, topotecan, toremifene, tositumomab, trabectedin, trametinib, tramadol, trastuzumab, trastuzumab emtansine, treosulfan, tretinoin, trifluridine+tipiracil, trilostane, triptorelin, trametinib, trofosfamide, thrombopoietin, tryptophan, ubenimex, valatinib, valrubicin, vandetanib, vapreotide, vemurafenib, vinblastine, vincristine, vindesine, vinflunine, vinorelbine, vismodegib, vorinostat, vorozole, yttrium-90 glass microspheres, zinostatin, zinostatin stimalamer, zoledronic acid and zorubicin.

Based upon standard laboratory techniques known to evaluate compounds useful for the treatment of hyperproliferative disorders, by standard toxicity tests and by standard pharmacological assays for the determination of treatment of the conditions identified above in mammals, and by comparison of these results with the results of known active ingredients or medicaments that are used to treat these conditions, the effective dosage of the compounds of the present invention can readily be determined for treatment of each desired indication. The amount of the active ingredient to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredient to be administered will generally range from about 0.001 mg/kg to about 200 mg/kg body weight per day, and preferably from about 0.01 mg/kg to about 20 mg/kg body weight per day. Clinically useful dosing schedules will range from one to three times a day dosing to once every four weeks dosing. In addition, it is possible for “drug holidays”, in which a patient is not dosed with a drug for a certain period of time, to be beneficial to the overall balance between pharmacological effect and tolerability. It is possible for a unit dosage to contain from about 0.5 mg to about 1500 mg of active ingredient, and can be administered one or more times per day or less than once a day. The average daily dosage for administration by injection, including intravenous, intramuscular, subcutaneous and parenteral injections, and use of infusion techniques will preferably be from 0.01 to 200 mg/kg of total body weight. The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg. The average daily inhalation dosage regimen will preferably be from 0.01 to 100 mg/kg of total body weight.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

### Experimental Section

### General Remarks

In the text, the chemical names and the numbers of the compounds are given in bold. The intermediates are defined by INT-.

Chemical names were generated using the ACD/Name software from ACD/Labs. In some cases generally accepted names of commercially available reagents were used in place of ACD/Name generated names.

If the purity of the obtained example product is not mentioned, the compounds are 90 to 100% pure.

The ¹H-NMR data of selected compounds are listed in the form of ¹H-NMR peaklists. Therein, for each signal peak the δ value in ppm is given, followed by the signal intensity, reported in round brackets. The δ value-signal intensity pairs from different peaks are separated by commas. Therefore, a peaklist is described by the general form: δ₁ (intensity₁), δ₂ (intensity₂), . . . , δᵢ (intensityᵢ), . . . , δₙ (intensityₙ).

The intensity of a sharp signal correlates with the height (in cm) of the signal in a printed NMR spectrum. When compared with other signals, this data can be correlated to the real ratios of the signal intensities. In the case of broad signals, more than one peak, or the center of the signal along with their relative intensity, compared to the most intense signal displayed in the spectrum, are shown. A ¹H-NMR peaklist is similar to a classical ¹H-NMR readout, and thus usually contains all the peaks listed in a classical NMR interpretation. Moreover, similar to classical ¹H-NMR printouts, peaklists can show solvent signals, signals derived from stereoisomers of the particular target compound, peaks of impurities, ¹³C satellite peaks, and/or spinning sidebands. The peaks of stereoisomers, and/or peaks of impurities are typically displayed with a lower intensity compared to the peaks of the target compound (e.g., with a purity of >90%). Such stereoisomers and/or impurities may be typical for the particular manufacturing process, and therefore their peaks may help to identify a reproduction of the manufacturing process on the basis of “by-product fingerprints”. An expert who calculates the peaks of the target compound by known methods (MestReC, ACD simulation, or by use of empirically evaluated expectation values), can isolate the peaks of the target compound as required, optionally using additional intensity filters. Such an operation would be similar to peak-picking in classical ¹H-NMR interpretation. A detailed description of the reporting of NMR data in the form of peaklists can be found in the publication “Citation of NMR Peaklist Data within Patent Applications” (cf. http://www.researchdisclosure.com/searching-disclosures, Research Disclosure Database Number 605005, 2014, 1 Aug. 2014). In the peak picking routine, as described in the Research Disclosure Database Number 605005, the parameter “MinimumHeight” can be adjusted between 1% and 4%. However, depending on the chemical structure and/or depending on the concentration of the measured compound it may be reasonable to set the parameter “MinimumHeight”<1%.

Chemical names were generated using the ACD/Name software from ACD/Labs. In some cases generally accepted names of commercially available reagents were used in place of ACD/Name generated names.

The following table 1 lists the abbreviations used in this paragraph and in the Examples section as far as they are not explained within the text body. Other abbreviations have their meanings customary per se to the skilled person.

TABLE 1
Abbreviations

| Abbreviation | Meaning |
|---|---|
| h | hour(s) |
| rt | room temperature |
| aq. | aqueous |
| sat. | saturated |
| HCl | hydrochloric acid |
| NaHCO₃ | sodium bicarbonate |
| DMF | N,N-dimethylformamide |
| DMSO | dimethyl sulfoxide |
| THF | tetrahydrofuran |
| HCOOH | formic acid |
| DIPEA | N,N-diisopropylamine |
| PyBOP | [(1H-benzotriazol-1-yl)oxy]tri(pyrrolidin-1- |
| | yl)phosphanium hexafluoridophosphate(1-) |
| CDI | 1,1′-Carbonyldiimidazole |
| DBU | 1,8-Diazabicyclo(5.4.0)undec-7-en |
| DMAP | 4-Dimethylaminopyridine |
| XPhos Pd G2 | (2′-amino[biphenyl]-2-yl)(chloro)palladium- |
| | dicyclohexyl(2′,4′,6′-triisopropyl[biphenyl]-3- |
| | yl)phosphine (1:1) |
| LC | liquid chromatography |
| MS | mass spectrostrometry |
| HPLC | high performance liquid chromatography |
| UPLC | ultra performance liquid chromatography |
| DAD | diode array detector |
| NMR | nuclear magnetic resonance spectroscopy: chemical |
| PyBOP | Benzotriazol-1-yl-oxytripyrrolidinophosphonium |
| | hexafluorophosphate |
| RT | room temperature |
| Rt | retention time (as measured either with HPLC or UPLC) |
| rac | racemic |
| THF | tetrahydrofuran |
| TPP | Triphenylphospine |
| UPLC | ultra performance liquid chromatography |

The following table lists the abbreviations used herein.

shifts (δ) are given in ppm. The chemical shifts were corrected by setting the DMSO signal to 2.50 ppm unless otherwise stated.

Other abbreviations have their meanings customary per se to the skilled person.

### Experimental Section—General Part

All reagents, for which the synthesis is not described in the experimental part, are either commercially available, or are known compounds or may be formed from known compounds by known methods by a person skilled in the art.

The compounds and intermediates produced according to the methods of the invention may require purification. Purification of organic compounds is well known to the person skilled in the art and there may be several ways of purifying the same compound. In some cases, no purification may be necessary. In some cases, the compounds may be purified by crystallization. In some cases, impurities may be stirred out using a suitable solvent. In some cases, the compounds may be purified by chromatography, particularly flash column chromatography, using for example prepacked silica gel cartridges, e.g. Biotage SNAP cartridges KP-Sil® or KP-NH® in combination with a Biotage autopurifier system (SP4® or Isolera Four®) and eluents such as gradients of hexane/ethyl acetate or DCM/methanol. In some cases, the compounds may be purified by preparative HPLC using for example a Waters autopurifier equipped with a diode array detector and/or on-line electrospray ionization mass spectrometer in combination with a suitable prepacked reverse phase column and eluents such as gradients of water and acetonitrile which may contain additives such as trifluoroacetic acid, formic acid or aqueous ammonia.

In some cases, purification methods as described above can provide those compounds of the present invention which possess a sufficiently basic or acidic functionality in the form of a salt, such as, in the case of a compound of the present invention which is sufficiently basic, a trifluoroacetate or formate salt for example, or, in the case of a compound of the present invention which is sufficiently acidic, an ammonium salt for example. A salt of this type can either be transformed into its free base or free acid form, respectively, by various methods known to the person skilled in the art, or be used as salts in subsequent biological assays. It is to be understood that the specific form (e.g. salt, free base etc.) of a compound of the present invention as isolated and as described herein is not necessarily the only form in which said compound can be applied to a biological assay in order to quantify the specific biological activity.

### Solvents

The following solvents were purchased from commercial sources and were used without purification:
N,N-dimethylformamide (CAS: 68-12-2)
Tetrahydrofuran (CAS: 109-99-9)
Dichloromethane (CAS: 75-09-2)
Dimethyl sulfoxide (CAS: 67-68-5)
Ethyl acetate (CAS: 141-78-6)
Hydrochloric acid (CAS: 7647-01-0
Hexane (CAS: 110-54-3)
Acetonitrile (CAS: 75-05-8)
Formic acid (CAS: 64-18-6)
Isopropanol (CAS: 67-63-0)
N,N-dimethylacetamide (CAS: 127-19-5)
Ammonium hydroxide (CAS: 1336-21-6)
1,4-Dioxane (CAS: 123-91-1)

### Reagents

All reagents, for which the synthesis is not described in the experimental part, are either commercially available or synthesized according to literature procedures.

The following reactants were purchased from commercial sources and were used without further purification:
(2′-Aminobiphenyl-2-yl)(chloro)palladium-dicyclohexyl[2′,4′,6′-tri(propan-2-yl)biphenyl-3-yl]phosphane (1:1) (Palladium-Xphos G2) (CAS: 1310584-14-5)
(Benzotriazol-1-yl-oxytripyrrolidinophosphonium hexafluorophosphate PyBOP (CAS: 128625-52-5)
Bis(triphenylphosphine)palladium(II) dichloride (CAS: 13965-03-2)
N,N-diisopropylethylamine (CAS: 7087-68-5)
1,1-dimethoxy-N,N-dimethylmethanamine (CAS: 4637-24-5)
cesium carbonate (CAS: 534-17-8)
Copper(I) iodide (CAS: 7681-65-4)
lithium hydroxide (CAS: 1310-65-2)
Palladium (II)acetate (CAS: 3375-31-3)
sodium carbonate (CAS: 497-19-8)
tetrakis(triphenylphosphin)palladium(0) (CAS: 14221-01-3)
1,1′-Bis(diphenylphosphino)ferrocene (CAS: 12150-46-8)

### Analytical LC-MS Methods

### Method 1:

Instrument: Waters Acquity UPLCMS SingleQuad; Column: Acquity UPLC BEH C₁₈ 1.7 μm, 50×2.1 mm; eluent A: water+0.1 vol % formic acid (99%), eluent B: acetonitrile; gradient: 0-1.6 min 1-99% B, 1.6-2.0 min 99% B; flow 0.8 ml/min; temperature: 60° C.; DAD scan: 210-400 nm.

method 2:

Instrument: Waters Acquity UPLCMS SingleQuad; Column: Acquity UPLC BEH C₁₈ 1.7 μm, 50×2.1 mm; eluent A: water+0.2 vol % aqueous ammonia (32%), eluent B: acetonitrile; gradient: 0-1.6 min 1-99% B, 1.6-2.0 min 99% B; flow 0.8 ml/min; temperature: 60° C.; DAD scan: 210-400 nm.

method 3:

Instrument: Waters Acquity UPLCMS SingleQuad; Column: Acquity UPLC BEH C₁₈ 1.7 50×2.1 mm; eluent A: water+0.1 vol % formic acid (99%), eluent B: acetonitrile; gradient: 0-1.6 min 1-99% B, 1.6-2.0 min 99% B; flow 0.8 ml/min; temperature: 60° C.; DAD scan: 210-400 nm

method 4:

Instrument: Waters Acquity; Column: Waters Acquity BEH C₁₈ 50 mm×2.1 mm×1.7 μm; eluent A: Water (MilliQ)+0.01 vol % formic acid, eluent B: acetonitrile+0.01 vol % formic acid; gradient: 0-0.3 min 3-4% B, 0.3-1.5 min 4-95% B, 1.5-1.9 min 95% B; 1.9-2.0 min 5% B; flow: 0.65 mL/min; temperature: 50° C.; DAD scan: 200-500 nm.

method 5:

Instrument: Waters Acquity; Column: Waters Acquity BEH C₁₈ 50 mm×2.1 mm×1.7 μm; eluent A: Water (MilliQ)+0.01 vol % formic acid, eluent B: acetonitrile+0.01 vol % formic acid; gradient: 0-0.5 min 5% B, 0.5-4.0 min 5-95% B, 4.0-4.5 min 95% B; 4.5-5.0 min 5% B; flow: 0.65 mL/min; temperature: 50° C.; DAD scan: 200-500 nm.

### Method 6:

Instrument: Waters Autopurification MS SingleQuad; Column: Waters XBrigde C₁₈ 5 μm 100×30 mm; water+0.1 vol-% formic acid (99%), Eluent B: Acetonitrile; Gradient: 0-5.5 min 5-100% B; flow 70 mL/min; Temperature: 25° C.; DAD scan: 210-400 nm.

### Method 7:

Instrument: Agilent 1290 UPLCMS 6230 TOF; Column: BEH C₁₈ 1.7 μm, 50×2.1 mm; Eluent A: Wasser+0.05% formic acid (99%); Eluent B: ecetonitrile+0.05% formic acid (99%); Gradient: 0-1.7 2-90% B, 1.7-2.0 90% B; flow 1.2 mL/min; temperature: 60° C.; DAD scan: 190-400 nm

### Preparative HPLC Methods

### Methods Used Depending on Retention Time of Desired Product

### Instrument Description:

Instrument: pump: Labomatic HD-5000 or HD-3000, head HDK 280, lowpressure gradient module ND-B1000; manual injection valve: Rheodyne 3725i038; detector: Knauer Azura UVD 2.15; collector: Labomatic Labocol Vario-4000; column: Chromatorex RP C-18 10 μm, 125×30 mm.

### Eluent Acidic:

solvent A: water+0.1 vol-% formic acid, solvent B: acetonitrile;

### Eluent Basic:

solvent A: water+0.2 vol-% ammonia (32%), solvent B: acetonitrile;

### General Procedures (GP)

### GP5: Coupling Between a Carboxylic Acid and a Sulfonamide

The intermediates and examples were synthesized using two different coupling methods:

### A/ Using PyBOP as a Coupling Agent (GP5A)

The sulfonamide partner (1.2 eq.), the acid partner (1.0 eq) and the commercially available coupling agent PyBOP (1.2 eq.) were suspended in DCM (0.1 mL/mmol) under argon atmosphere. After degassing with argon for 5 min, DIPEA (4.0 eq.) was added and the mixture was stirred at RT for 16 h-3 d. After reaction completion, the mixture was diluted with DCM, water was added and the layers were separated. The aqueous layer was extracted with DCM. The combined organic layers were washed with brine and dried with sodium sulfate. After filtration, the solvent was removed under vacuum. The residue was purified by HPLC HT or column chromatography to give the desired sulfonamide. Workup B: Half conc. brine and DCM were added to the reaction mixture and the layers were separated. The aqueous layer was extracted. The combined organic layers were dried over a hydrophobic filter and evaporated.

### EXPERIMENTAL SECTION—INTERMEDIATES

### Intermediate 1

### 4-(Azetidin-1-yl)-2-fluoro-6-hydroxybenzaldehyde

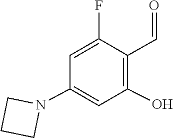 [SMILES: O=Cc1c(O)cc(N2CCC2)cc1F]

To a stirring solution of 2,4-difluoro-6-hydroxybenzaldehyde (36.9 g, 234 mmol) in 1,4-dioxane (400 ml) was added N,N-diisopropylethylamine (75.5 g, 101.7 ml, 584 mmol), followed by the addition of azetidine (20.0 g, 350 mmol). The resulting solution was stirred at 60° C. overnight. The reaction mixture was allowed to cool to room temperature diluted with dichloromethane and water and the undissolved, oily, solid was filtered off. The layers were separated, the aqueous layer was extracted with dichloromethane one more time and the combined organic layers were washed with aq., sat. NaCl solution, dried over sodium sulfate and concentrated in vacuo. The residue was purified by column chromatography (hexane/ethyl acetate) to give 24.1 g (53% yield) of the title compound as an orange solid. ¹H-NMR (400 MHz, DMSO-d₆) δ [ppm]: 1.170 (0.41), 1.985 (0.70), 2.304 (1.33), 2.323 (3.20), 2.332 (0.94), 2.342 (5.86), 2.345 (2.25), 2.352 (0.88), 2.361 (3.42), 2.379 (1.44), 2.518 (0.55), 3.977 (12.41), 3.986 (1.53), 3.996 (16.00), 4.005 (1.41), 4.015 (12.29), 5.544 (5.70), 5.550 (5.77), 5.800 (3.94), 5.805 (3.93), 5.834 (4.24), 5.839 (4.08), 9.745 (12.47); LC-MS (method 1): Rt=1.12 min; MS (ESIpos): m/z=196 [M+H]⁺

### Intermediate 2

### Ethyl 6-(azetidin-1-yl)-4-fluoro-1-benzofuran-2-carboxylate

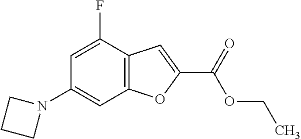 [SMILES: CCOC(=O)c1cc2c(F)cc(N3CCC3)cc2o1]

To a stirring solution of Intermediate 1 (24.1 g, 123 mmol) and ethyl chloroacetate (14 ml, 130 mmol) in DMF (300 mL) was added potassium carbonate (171 g, 1.23 mol; CAS-RN:[584-08-7]) and the resulting mixture was stirred at 160° C. for 1 h. The reaction mixture was poured into water at room temperature, carefully acidified with conc. HCl and extracted with ethyl acetate twice. The combined organic layers were dried over sodium sulfate and concentrated in vacuo to give 34.8 g (107% yield) of a mixture of ester, acid and not cyclized product as a brown oil which was used without purification.

### Intermediate 3

### 6-(Azetidin-1-yl)-4-fluoro-1-benzofuran-2-carboxylic acid

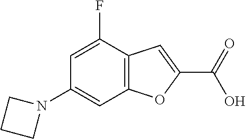 [SMILES: O=C(O)c1cc2c(F)cc(N3CCC3)cc2o1]

To a stirring solution of ethyl 6-(azetidin-1-yl)-4-fluoro-1-benzofuran-2-carboxylate (Intermediate 2, 34.8 g, 132 mmol) in 1,4-dioxane (500 ml) was added water (200 ml) and lithium hydroxide (9.49 g, 396 mmol; CAS-RN:[1310-65-2]) and the resulting mixture was stirred at room temperature for 90 min. The reaction mixture was concentrated in vacuo, the residue was diluted with 300 mL water and acidified to pH 5 with aq. HCl (1 M). The resulting precipitate was stirred overnight, collected by filtration, washed with 50 mL Water and dried in vacuo at 50° C. to give 28.5 g (92% yield) of the title compound as a light brown solid. ¹H-NMR (400 MHz, DMSO-d₆) δ [ppm]: 1.983 (0.80), 1.989 (0.44), 1.999 (1.23), 2.016 (0.82), 2.294 (1.11), 2.302 (0.77), 2.311 (3.13), 2.319 (1.25), 2.331 (4.85), 2.342 (0.96), 2.349 (3.42), 2.367 (1.16), 2.518 (1.11), 2.523 (0.74), 2.940 (0.53), 3.186 (0.54), 3.203 (0.91), 3.217 (0.95), 3.233 (0.65), 3.731 (1.22), 3.747 (2.45), 3.763 (1.16), 3.802 (0.91), 3.821 (1.28), 3.838 (0.90), 3.870 (10.30), 3.877 (1.66), 3.888 (16.00), 3.899 (1.58), 3.906 (9.67), 6.294 (3.56), 6.298 (3.59), 6.323 (3.38), 6.328 (3.69), 6.408 (5.37), 6.411 (6.54), 6.415 (4.29), 6.471 (0.53), 6.475 (0.57), 6.502 (0.55), 6.506 (0.57), 6.542 (0.57), 6.558 (0.96), 6.561 (1.11), 6.564 (0.78), 7.477 (1.72), 7.480 (1.66), 7.522 (10.28), 7.525 (9.19), 7.738 (0.54), 7.744 (0.53); LC-MS (method 1): Rt=1.04 min; MS (ESIpos): m/z=236 [M+H]⁺

### Intermediate 4

### Methyl 6-(azetidin-1-yl)-1-benzofuran-2-carboxylate

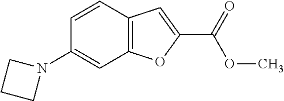 [SMILES: COC(=O)c1cc2ccc(N3CCC3)cc2o1]

Methyl 6-bromo-1-benzofuran-2-carboxylate (2.50 g, 9.80 mmol), Chloro(2-dicyclohexylphosphino-2′,4′,6′-triisopropyl-1,1′-biphenyl)[2-(2′-amino-1,1′-biphenyl)]palladium(II) (771 mg, 980 μmol) and cesium carbonate (7.98 g, 24.5 mmol; CAS-RN:[534-17-8]) were suspended in 1,4-dioxane (42 ml). Azetidine hydrogen chloride (1/1) (1.10 g, 11.8 mmol) was added and the resulting reaction mixture was stirred at 110° C. for 4 h. Water was added and the mixture was extracted with ethyl acetate twice. The combined organic layers were dried over a water resistant filter and concentrated in vacuo. The residue was purified by column chromatography (hexane/ethyl acetate, 10%- 40% EE) to give 350 mg (15% yield) of the title compound as a light yellow solid. ¹H-NMR (400 MHz, DMSO-d₆) δ [ppm]: 2.319 (1.18), 2.327 (0.68), 2.332 (0.57), 2.338 (1.70), 2.356 (1.16), 2.374 (0.40), 2.518 (1.87), 2.523 (1.28), 2.669 (0.45), 3.834 (16.00), 3.870 (3.68), 3.888 (5.52), 3.899 (0.44), 3.906 (3.49), 6.481 (1.33), 6.487 (1.44), 6.502 (1.29), 6.507 (1.62), 6.558 (1.57), 7.527 (2.09), 7.548 (2.04), 7.596 (3.56), 7.599 (3.36); LC-MS (method 1): Rt=1.20 min; MS (ESIpos): m/z=232 [M+H]⁺

### Intermediate 5

### 6-(Azetidin-1-yl)-1-benzofuran-2-carboxylic acid

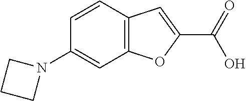 [SMILES: O=C(O)c1cc2ccc(N3CCC3)cc2o1]

Intermediate 4 (350 mg, 1.51 mmol) was dissolved in THF (13 ml), followed by the addition of lithium hydroxide (181 mg, 7.57 mmol; CAS-RN:[1310-65-2]) in water (6.4 ml). The mixture was stirred for 4 h at room temperature. THF was evaporated. The aqueous residue was acidified with half concentrated HCl. The aqueous layer was evaporated to give 210 mg (64% yield) of the title compound as a yellow solid. The crude was used without purification. ¹H-NMR (400 MHz, DMSO-d₆) δ [ppm]: 1.137 (0.80), 1.222 (0.50), 1.348 (0.61), 2.074 (0.44), 2.083 (7.60), 2.295 (1.03), 2.312 (3.06), 2.322 (0.98), 2.331 (4.87), 2.349 (3.41), 2.367 (1.11), 2.518 (1.53), 2.522 (1.07), 2.539 (1.26), 3.332 (1.09), 3.518 (0.52), 3.859 (10.28), 3.865 (1.46), 3.877 (16.00), 3.887 (1.38), 3.894 (9.75), 6.461 (3.73), 6.466 (4.27), 6.482 (3.93), 6.487 (4.56), 6.541 (4.85), 6.544 (4.78), 6.546 (4.19), 7.371 (0.43), 7.393 (0.45), 7.424 (0.70), 7.426 (0.70), 7.433 (0.46), 7.485 (9.19), 7.487 (8.47), 7.506 (6.42), 7.527 (6.04); LC-MS (method 1): Rt=0.91 min; MS (ESIpos): m/z=218 [M+H]⁺

### Intermediate 6

### Methyl 6-(3-fluoroazetidin-1-yl)-1-benzofuran-2-carboxylate

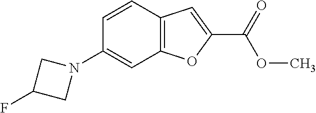 [SMILES: COC(=O)c1cc2ccc(N3CC(F)C3)cc2o1]

Synthesized analogously to Intermediate 4 using 3-fluoroazetidine hydrogen chloride (1/1) (131 mg, 1.18 mmol) instead of Azetidine hydrogen chloride (1/1) and purified by HT-HPLC to give 150 mg (55% yield) of the title compound as a yellow solid. ¹H-NMR (400 MHz, DMSO-d₆) δ [ppm]: 2.087 (2.57), 2.521 (1.60), 2.526 (1.01), 3.340 (16.00), 3.926 (2.31), 3.929 (2.46), 3.933 (2.60), 3.937 (2.34), 3.950 (2.81), 3.953 (3.07), 3.958 (3.07), 3.961 (2.97), 3.986 (2.50), 3.990 (2.58), 3.994 (2.62), 3.997 (2.36), 4.011 (2.83), 4.014 (3.08), 4.018 (2.94), 4.021 (2.74), 4.189 (2.62), 4.192 (2.55), 4.203 (3.14), 4.206 (2.92), 4.213 (2.42), 4.216 (2.32), 4.227 (2.44), 4.230 (2.37), 4.241 (2.73), 4.244 (2.65), 4.255 (2.95), 4.258 (2.80), 4.265 (2.47), 4.268 (2.45), 4.279 (2.27), 4.282 (2.19), 5.430 (0.60), 5.438 (1.23), 5.445 (1.57), 5.452 (2.25), 5.460 (1.52), 5.467 (1.10), 5.475 (0.50), 5.574 (0.57), 5.582 (1.13), 5.589 (1.60), 5.596 (2.26), 5.603 (1.52), 5.611 (1.19), 5.618 (0.54), 6.558 (6.07), 6.563 (6.40), 6.580 (6.12), 6.585 (6.55), 6.690 (7.44), 7.569 (9.83), 7.591 (9.25), 7.622 (14.79), 7.624 (14.98), 8.540 (0.95); LC-MS (method 1): Rₜ=1.13 min; MS (ESIpos): m/z=250 [M+H]⁺

### Intermediate 7

### 6-(3-Fluoroazetidin-1-yl)-1-benzofuran-2-carboxylic acid

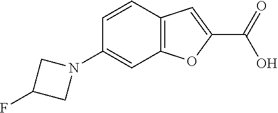 [SMILES: O=C(O)c1cc2ccc(N3CC(F)C3)cc2o1]

Intermediate 6 (150 mg, 602 μmol) was dissolved in THF (5.1 ml), followed by the addition of lithium hydroxide (72.1 mg, 3.01 mmol; CAS-RN:[1310-65-2]) in water (2.6 ml). The mixture was stirred for 4 h at room temperature. THF mixture was evaporated and the aqueous residue was acidified with half concentrated HCl. The resulting precipitate was collected by filtration and dried in vacuo to give 65.0 mg (44% yield) of the title compound as a yellow solid. LC-MS (method 1): Rₜ=0.90 min; MS (ESIpos): m/z=236 [M+H]⁺

### Intermediate 8

### Methyl 6-(3-fluoro-3-methylazetidin-1-yl)-1-benzofuran-2-carboxylate

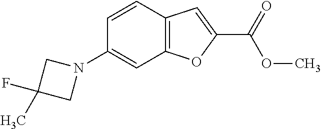 [SMILES: COC(=O)c1cc2ccc(N3CC(C)(F)C3)cc2o1]

Synthesized analogously to

Intermediate 6 using commercially available 3-fluoro-3-methylazetidine-hydrogen chloride (1/1) (59.1 mg, 470 μmol) instead of 3-fluoroazetidine hydrogen chloride (1/1) to give 30.0 mg (26% yield) of the title compound as a brown solid. ¹H-NMR (400 MHz, CHLOROFORM-d) 5 [ppm]: 1.524 (5.21), 3.749 (1.26), 3.772 (2.15), 3.781 (16.00), 3.788 (2.09), 3.812 (2.04), 3.816 (3.06), 3.861 (1.63), 3.883 (1.15), 3.913 (1.61), 3.935 (1.16), 6.300 (1.30), 6.305 (1.47), 6.321 (1.32), 6.326 (1.52), 6.380 (2.03), 7.109 (0.64), 7.264 (0.68), 7.272 (3.91), 7.280 (0.50), 7.284 (0.40), 7.303 (2.41), 7.324 (2.44), 7.368 (0.45); LC-MS (method 3): Rₜ=1.18 min; MS (ESIpos): m/z=264 [M+H]⁺

### Intermediate 9

### 6-(3-Fluoro-3-methylazetidin-1-yl)-1-benzofuran-2-carboxylic acid

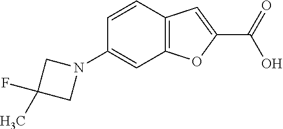 [SMILES: CC1(F)CN(c2ccc3cc(C(=O)O)oc3c2)C1]

Synthesized analogously to

Intermediate 7 using Intermediate 8, methyl 6-(3-fluoro-3-methylazetidin-1-yl)-1-benzofuran-2-carboxylate (30.0 mg, 114 μmol) instead of

Intermediate 6 and worked up accordingly to give 25.0 mg (84% yield) of the title compound as a white solid. LC-MS (method 1): Rₜ=1.00 min; MS (ESIpos): m/z=250 [M+H]⁺

### Intermediate 10

### Methyl 6-[3-(trifluoromethyl)azetidin-1-yl]-1-benzofuran-2-carboxylate

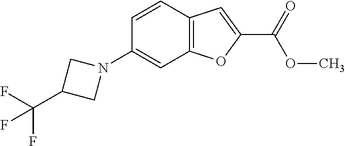 [SMILES: COC(=O)c1cc2ccc(N3CC(C(F)(F)F)C3)cc2o1]

Synthesized analogously to Intermediate 4 using commercially available 3-(trifluoromethyl)azetidine (206 mg, 1.65 mmol) instead of azetidine hydrogen chloride (1/1), worked up accordingly and purified by HT-HPLC to give 110 mg (95% purity, 25% yield) of the title compound as a white solid. ¹H-NMR (400 MHz, DMSO-de) b [ppm]: 1.294 (0.60), 2.084 (0.59), 2.518 (2.28), 2.523 (1.52), 3.600 (0.43), 3.832 (0.60), 3.843 (16.00), 3.929 (1.48), 3.942 (1.34), 3.950 (1.80), 3.963 (1.48), 4.112 (1.81), 4.133 (3.19), 4.154 (1.29), 6.569 (1.45), 6.574 (1.49), 6.590 (1.46), 6.596 (1.55), 6.722 (1.71), 7.580 (2.26), 7.601 (2.18), 7.624 (3.49), 7.627 (3.85); LC-MS (method 1): Rₜ=1.26 min; MS (ESIpos): m/z=300 [M+H]⁺

### Intermediate 11

### 6-[3-(Trifluoromethyl)azetidin-1-yl]-1-benzofuran-2-carboxylic acid

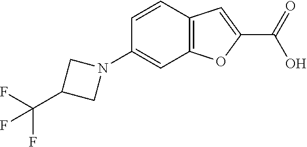 [SMILES: O=C(O)c1cc2ccc(N3CC(C(F)(F)F)C3)cc2o1]

Synthesized analogously to Intermediate 5 using Intermediate 10, methyl 6-[3-(trifluoromethyl)azetidin-1-yl]-1-benzofuran-2-carboxylate (110 mg, 368 μmol) instead of Intermediate 4 and worked up accordingly to give 95.0 mg (82% yield) of the title compound as a white solid. LC-MS (method 1): Rₜ=1.06 min; MS (ESIpos): m/z=286 [M+H]⁺

### Intermediate 12

### Methyl 6-(3,3-difluoroazetidin-1-yl)-1-benzofuran-2-carboxylate

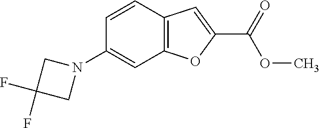 [SMILES: COC(=O)c1cc2ccc(N3CC(F)(F)C3)cc2o1]

Synthesized analogously to Intermediate 4 using commercially available 3,3-difluoroazetidine-hydrogen chloride (1/1) (305 mg, 2.35 mmol) instead of Azetidine hydrogen chloride and worked up accordingly to give 200 mg (36% yield) of the title compound as an ochre solid. ¹H-NMR (400 MHz, DMSO-d₆) δ [ppm]: 3.337 (16.00), 4.335 (3.96), 4.365 (8.31), 4.396 (3.94), 6.643 (1.55), 6.648 (1.61), 6.664 (1.56), 6.669 (1.67), 6.822 (2.03), 7.620 (2.66), 7.641 (2.57), 7.652 (3.97), 7.654 (3.71); LC-MS (method 1): Rₜ=1.19 min; MS (ESIpos): m/z=268 [M+H]⁺

### Intermediate 13

### 6-(3,3-Difluoroazetidin-1-yl)-1-benzofuran-2-carboxylic acid

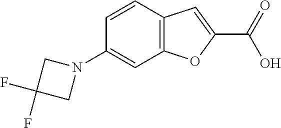 [SMILES: O=C(O)c1cc2ccc(N3CC(F)(F)C3)cc2o1]

Synthesized analogously to Intermediate 5 using Intermediate 12, methyl 6-(3,3-difluoroazetidin-1-yl)-1-benzofuran-2-carboxylate (200 mg, 748 μmol) instead of Intermediate 4 and worked up accordingly to give 175 mg (91% purity, 84% yield) of the title compound as a light green solid. LC-MS (method 1): Rₜ=0.98 min; MS (ESIpos): m/z=254 [M+H]⁺

### Intermediate 14

### 6-(Azetidin-1-yl)-4-fluoro-1-benzofuran-2-carboxamide

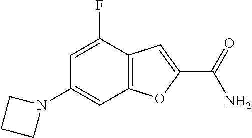 [SMILES: NC(=O)c1cc2c(F)cc(N3CCC3)cc2o1]

To a stirring solution of 6-(azetidin-1-yl)-4-fluoro-1-benzofuran-2-carboxylic acid (3.35 g, 14.2 mmol) in DMF (20.0 ml) was added Propanephosphonic acid anhydride (10 ml, 50% purity, 17 mmol; CAS-RN:[68957-94-8]) and N,N-diisopropylethylamine (9.9 ml, 57 mmol; CAS-RN:[7087-68-5]) followed by the dropwise addition of (6.7 mL, 25% purity, 43 mmol; CAS-RN:[1336-21-6]). The resulting mixture was stirred for 20 h at room temperature. The resulting precipitate was collected by filtration stirred in Methyl tert-butyl ether for 30 min, collected by filtration and dried in vacuo to give 2.50 g (67% yield) of the title compound as a white solid. ¹H-NMR (400 MHz, DMSO-d₆) δ [ppm]: 0.883 (2.45), 0.901 (5.31), 0.904 (5.51), 0.922 (2.69), 1.286 (0.78), 1.308 (1.19), 1.349 (1.02), 1.418 (1.23), 2.310 (3.04), 2.327 (6.64), 2.345 (3.40), 2.364 (1.23), 2.518 (16.00), 2.522 (11.54), 2.669 (2.42), 2.673 (1.91), 2.729 (9.57), 2.888 (12.05), 3.802 (2.25), 3.821 (3.27), 3.839 (2.19), 3.858 (9.59), 3.877 (15.37), 3.895 (9.24), 6.141 (0.66), 6.174 (0.67), 6.277 (3.29), 6.281 (3.44), 6.307 (3.80), 6.311 (4.35), 6.346 (6.09), 6.755 (1.03), 7.441 (9.80), 7.443 (9.57), 7.538 (1.39), 7.897 (1.51), 7.950 (1.58); LC-MS (method 1): Rₜ=0.95 min; MS (ESIpos): m/z=235 [M+H]⁺

### Intermediate 15

### 6-(azetidin-1-yl)-N-(5-bromo-2-methoxybenzene-1-sulfonyl)-4-fluoro-1-benzofuran-2-carboxamide

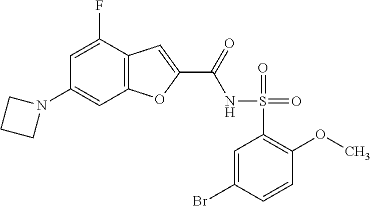 [SMILES: COc1ccc(Br)cc1S(=O)(=O)NC(=O)c1cc2c(F)cc(N3CCC3)cc2o1]

To a stirring solution of 6-(azetidin-1-yl)-4-fluoro-1-benzofuran-2-carboxylic acid (1.50 g, 6.38 mmol) in THF (30 ml) was added 1,1′-Carbonyldiimidazol (1.34 g, 8.29 mmol; CAS-RN:[530-62-1]) and the mixture was stirred at room temperature for 90 min. 5-bromo-2-methoxybenzene-1-sulfonamide (2.55 g, 9.57 mmol) and 1,8-Diazabicyclo(5.4.0)undec-7-en (1.3 ml, 8.8 mmol; CAS-RN:[6674-22-2]) was added subsequently and the resulting solution was stirred at room temperature overnight. After completion the reaction mixture was evaporated and the residue diluted with water and acidified with aq. HCl (1 M). The solid was collected by filtration and stirred in ethyl acetate for 30 min. The remaining solid was collected by filtration, washed with ethyl acetate and dried in vacuo to give 2.17 g (63% yield) of the title compound as a yellow solid. ¹H-NMR (400 MHz, DMSO-d₆) δ [ppm]: 2.306 (0.93), 2.325 (1.43), 2.336 (0.44), 2.344 (1.02), 2.518 (0.68), 2.522 (0.43), 3.874 (16.00), 3.893 (4.92), 3.911 (2.74), 5.757 (2.92), 6.314 (1.08), 6.318 (1.33), 6.347 (4.38), 7.222 (2.26), 7.245 (2.42), 7.853 (1.48), 7.860 (1.62), 7.875 (1.27), 7.882 (1.48), 7.943 (3.76), 7.949 (3.26), 8.014 (1.94); LC-MS (method 1): Rₜ=1.32 min; MS (ESIpos): m/z=483 [M+H]⁺

### Intermediate 16

### 2-Fluoro-4-(3-fluoroazetidin-1-yl)-6-hydroxybenzaldehyde

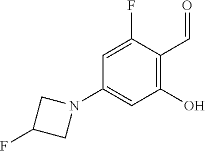 [SMILES: O=Cc1c(O)cc(N2CC(F)C2)cc1F]

Synthesized and purified analogously to Intermediate 1 using 3-fluoroazetidine hydrogen chloride (1/1) (3.00 g, 26.9 mmol) instead of azetidine to give 2.02 g (100% purity, 42% yield) of the title compound as a yellow solid. ¹H-NMR (400 MHz, DMSO-d₆) δ [ppm]: 4.042 (2.00), 4.047 (2.50), 4.049 (2.53), 4.054 (2.08), 4.070 (2.56), 4.074 (3.23), 4.077 (3.13), 4.081 (2.73), 4.103 (2.16), 4.108 (2.60), 4.110 (2.53), 4.115 (2.05), 4.130 (2.61), 4.135 (3.25), 4.142 (2.57), 4.280 (2.36), 4.284 (2.33), 4.294 (2.74), 4.298 (2.57), 4.306 (2.07), 4.311 (2.05), 4.321 (2.13), 4.325 (2.14), 4.333 (2.59), 4.337 (2.47), 4.348 (2.65), 4.352 (2.54), 4.360 (2.10), 4.365 (2.06), 4.375 (2.02), 4.379 (1.93), 5.416 (0.55), 5.423 (1.14), 5.431 (1.67), 5.438 (2.06), 5.446 (1.63), 5.453 (1.04), 5.460 (0.52), 5.560 (0.53), 5.567 (1.07), 5.574 (1.66), 5.582 (2.09), 5.589 (1.63), 5.596 (1.12), 5.604 (0.53), 5.670 (7.57), 5.674 (7.65), 5.915 (4.60), 5.920 (4.46), 5.948 (4.62), 5.953 (4.45), 9.805 (16.00), 11.941 (0.70); LC-MS (method 1): Rₜ=1.04 min; MS (ESIpos): m/z=214 [M+H]⁺

### Intermediate 17

### Ethyl 4-fluoro-6-(3-fluoroazetidin-1-yl)benzofuran-2-carboxylate

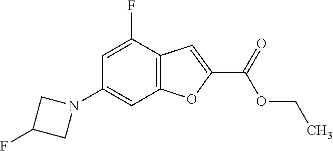 [SMILES: CCOC(=O)c1cc2c(F)cc(N3CC(F)C3)cc2o1]

Synthesized analogously to Intermediate 2 using 2-fluoro-4-(3-fluoroazetidin-1-yl)-6-hydroxybenzaldehyde (Intermediate 16, 16.8 g, 78.8 mmol) instead of Intermediate 1 to give 17.43 g (74% yield) of a mixture of ester, acid and not cyclized product as a brown oil which was used without purification.

### Intermediate 18

### 4-Fluoro-6-(3-fluoroazetidin-1-yl)-1-benzofuran-2-carboxylic acid

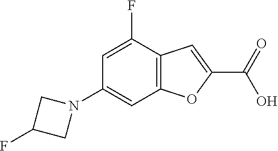 [SMILES: O=C(O)c1cc2c(F)cc(N3CC(F)C3)cc2o1]

Synthesized and crystallized analogously to

Intermediate 3 using ethyl 4-fluoro-6-(3-fluoroazetidin-1-yl)-1-benzofuran-2-carboxylate (Intermediate 17, 11.3 g, 40.2 mmol) instead of Intermediate 2 to give 8.57 g (76% yield) of the title compound as an off white solid. ¹H-NMR (400 MHz, DMSO-d₆) δ [ppm]: 2.518 (1.43), 2.522 (0.88), 2.939 (0.49), 3.394 (0.45), 3.861 (0.41), 3.865 (0.40), 3.869 (0.43), 3.873 (0.44), 3.885 (0.45), 3.889 (0.55), 3.893 (0.50), 3.896 (0.55), 3.922 (0.62), 3.929 (3.01), 3.932 (3.10), 3.936 (3.07), 3.940 (2.98), 3.945 (1.13), 3.953 (3.57), 3.957 (3.97), 3.961 (3.65), 3.965 (3.52), 3.989 (2.88), 3.993 (2.96), 3.997 (3.08), 4.000 (2.91), 4.014 (3.26), 4.017 (3.57), 4.022 (3.40), 4.025 (3.16), 4.126 (0.40), 4.141 (0.48), 4.144 (0.44), 4.168 (0.44), 4.182 (3.17), 4.185 (2.98), 4.196 (3.91), 4.200 (3.51), 4.206 (2.98), 4.209 (2.81), 4.221 (2.94), 4.224 (2.76), 4.234 (3.05), 4.238 (3.06), 4.249 (3.39), 4.252 (3.26), 4.259 (2.81), 4.262 (2.86), 4.273 (2.57), 4.277 (2.55), 5.416 (0.76), 5.425 (1.48), 5.432 (1.92), 5.439 (2.46), 5.446 (1.80), 5.453 (1.27), 5.461 (0.58), 5.560 (0.70), 5.568 (1.30), 5.575 (1.87), 5.582 (2.49), 5.590 (1.83), 5.597 (1.43), 5.604 (0.66), 6.306 (0.75), 6.311 (0.76), 6.335 (0.72), 6.340 (0.75), 6.398 (6.10), 6.402 (6.31), 6.427 (6.05), 6.431 (6.31), 6.523 (1.06), 6.528 (1.54), 6.533 (8.56), 6.535 (10.58), 6.537 (10.17), 6.540 (8.07), 6.651 (0.48), 6.803 (0.46), 6.805 (0.50), 6.808 (0.47), 6.811 (0.50), 6.872 (1.06), 6.874 (1.09), 6.877 (1.15), 6.880 (1.08), 7.509 (15.78), 7.511 (16.00), 7.693 (0.81), 7.699 (0.78), 7.775 (1.49), 7.780 (1.48); LC-MS (method 1): Rₜ=0.99 min; MS (ESIpos): m/z=254 [M+H]⁺

### Intermediate 19

### 4-[2-(Difluoromethyl)azetidin-1-yl]-2-fluoro-6-hydroxybenzaldehyde (rac)

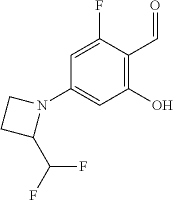 [SMILES: O=Cc1c(O)cc(N2CCC2C(F)F)cc1F]

Synthesized and purified analogously to Intermediate 1 using 2-(difluoromethyl)azetidine hydrogen chloride (1/1) (1.00 g, 6.97 mmol) instead of azetidine to give 466 mg (32% yield) of the title compound as an orange oil. ¹H-NMR (400 MHz, DMSO-d₆) δ [ppm]: 2.266 (0.62), 2.281 (1.08), 2.289 (0.91), 2.296 (1.59), 2.303 (1.33), 2.310 (1.62), 2.318 (1.84), 2.324 (1.33), 2.331 (1.77), 2.347 (0.83), 2.457 (0.70), 2.472 (0.94), 2.481 (2.09), 2.518 (2.39), 2.523 (2.05), 2.532 (0.67), 2.547 (0.53), 2.669 (0.43), 3.821 (1.25), 3.837 (1.46), 3.843 (2.01), 3.858 (2.09), 3.866 (1.74), 3.882 (1.42), 4.018 (1.52), 4.033 (1.78), 4.040 (2.75), 4.054 (2.73), 4.061 (1.42), 4.076 (1.20), 4.612 (0.71), 4.618 (0.88), 4.626 (1.04), 4.631 (0.98), 4.639 (1.33), 4.648 (1.01), 4.653 (1.02), 4.661 (0.86), 4.668 (0.67), 5.798 (5.23), 5.803 (5.38), 5.991 (3.64), 5.996 (3.48), 6.024 (3.54), 6.029 (3.39), 6.245 (1.75), 6.253 (1.71), 6.383 (3.35), 6.391 (3.39), 6.521 (1.53), 6.529 (1.62), 9.848 (16.00); LC-MS (method 1): Rₜ=1.16 min; MS (ESIneg): m/z=244 [M−H]⁻

### Intermediate 20

### Ethyl 6-[2-(difluoromethyl)azetidin-1-yl]-4-fluoro-benzofuran-2-carboxylate (rac)

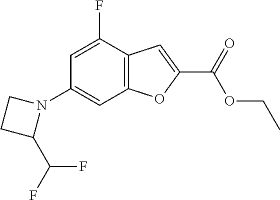 [SMILES: CCOC(=O)c1cc2c(F)cc(N3CCC3C(F)F)cc2o1]

Synthesized analogously to Intermediate 2 using 4-[2-(difluoromethyl)azetidin-1-yl]-2-fluoro-6-hydroxybenzaldehyde (Intermediate 19, 470 mg, 1.92 mmol) instead of Intermediate 1 to give 0.54 g (90% yield) of a mixture of ester, acid and not cyclized product as a brown oil which was used without purification.

### Intermediate 21

### 6-[2-(Difluoromethyl)azetidin-1-yl]-4-fluoro-benzofuran-2-carboxylic acid (rac)

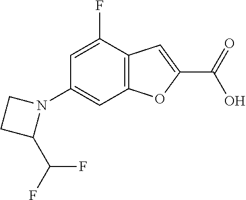 [SMILES: O=C(O)c1cc2c(F)cc(N3CCC3C(F)F)cc2o1]

Synthesized analogously to

Intermediate 3 using ethyl 6-[2-(difluoromethyl)azetidin-1-yl]-4-fluoro-benzofuran-2-carboxylate (rac) (Intermediate 20, 540 mg, 1.72 mmol) instead of Intermediate 2. The reaction mixture was concentrated in vacuo, the residue was diluted with 30 mL water and acidified to pH 5 with aq. HCl (1 M). Since there was no precipitate the mixture was extracted with dichloromethane/iso-propanol 4/1 twice. The combined organic layers were dried over sodium sulfate and concentrated in vacuo to give 388 mg (85% purity, 67% yield) of the title compound as a brown solid. ¹H-NMR (400 MHz, DMSO-d₆) δ [ppm]: 1.026 (0.52), 1.041 (0.52), 1.166 (0.60), 1.230 (0.90), 1.955 (3.89), 2.322 (0.84), 2.327 (1.16), 2.331 (0.99), 2.335 (0.94), 2.353 (1.30), 2.357 (1.35), 2.364 (2.01), 2.381 (3.17), 2.385 (2.78), 2.401 (4.02), 2.412 (2.16), 2.421 (3.39), 2.434 (2.87), 2.444 (2.00), 2.450 (1.70), 2.456 (1.97), 2.462 (1.59), 2.518 (3.44), 2.523 (2.25), 2.539 (2.57), 2.665 (0.78), 2.669 (1.07), 2.673 (0.78), 2.780 (3.77), 2.941 (5.56), 2.994 (0.46), 3.673 (0.64), 3.695 (0.66), 3.719 (1.92), 3.738 (4.28), 3.742 (3.15), 3.761 (4.20), 3.780 (1.91), 3.985 (0.53), 4.003 (2.46), 4.015 (2.95), 4.024 (3.47), 4.035 (3.76), 4.044 (2.27), 4.056 (1.89), 4.428 (0.51), 4.437 (0.60), 4.458 (1.43), 4.465 (1.52), 4.479 (1.94), 4.503 (1.44), 4.519 (0.53), 4.529 (0.40), 6.235 (2.46), 6.244 (2.37), 6.341 (0.57), 6.350 (0.59), 6.373 (4.82), 6.382 (4.21), 6.402 (0.75), 6.407 (0.77), 6.465 (6.34), 6.469 (6.57), 6.480 (0.57), 6.494 (6.36), 6.499 (6.59), 6.513 (2.10), 6.521 (2.18), 6.618 (1.22), 6.636 (9.73), 6.638 (11.73), 6.885 (1.01), 6.887 (1.07), 6.890 (1.05), 6.893 (1.03), 7.543 (16.00), 7.545 (15.52), 7.795 (1.60), 7.800 (1.57); LC-MS (method 1): Rₜ=1.08 min; MS (ESIpos): m/z=286 [M+H]⁺

### Intermediate 22

### 2-Fluoro-6-hydroxy-4-[2-(2-hydroxypropan-2-yl)azetidin-1-yl]benzaldehyde (rac)

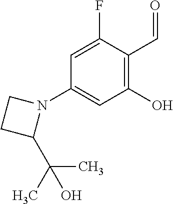 [SMILES: CC(C)(O)C1CCN1c1cc(O)c(C=O)c(F)c1]

Synthesized and purified analogously to Intermediate 1 using 2-(azetidin-2-yl)propan-2-ol hydrogen chloride (1/1) (1.00 g, 6.59 mmol) instead of azetidine to give 1.13 g (90% purity, 73% yield) of the title compound as an orange oil. ¹H-NMR (400 MHz, DMSO-d₆) δ [ppm]: 1.026 (2.37), 1.042 (2.54), 1.087 (14.64), 1.105 (0.57), 1.154 (4.37), 1.158 (16.00), 1.172 (4.34), 1.177 (0.65), 1.189 (2.14), 1.986 (7.70), 2.022 (0.57), 2.031 (0.45), 2.036 (0.67), 2.045 (0.74), 2.050 (0.80), 2.059 (0.70), 2.065 (0.50), 2.073 (0.68), 2.323 (0.40), 2.338 (0.41), 2.347 (0.67), 2.351 (0.46), 2.361 (0.70), 2.366 (0.57), 2.370 (0.61), 2.375 (0.59), 2.385 (0.52), 2.389 (0.62), 2.518 (0.56), 3.687 (0.56), 3.702 (0.67), 3.710 (1.03), 3.724 (1.06), 3.733 (0.76), 3.747 (0.62), 3.896 (0.59), 3.911 (0.70), 3.918 (1.19), 3.933 (1.17), 3.940 (0.58), 3.955 (0.46), 3.999 (0.52), 4.016 (1.61), 4.034 (1.61), 4.052 (0.53), 4.113 (1.15), 4.127 (1.25), 4.135 (1.26), 4.149 (1.09), 4.899 (2.58), 5.756 (5.51), 9.759 (7.67); LC-MS (method 1): Rₜ=1.06 min; MS (ESIpos): m/z=254 [M+H]⁺

### Intermediate 23

### Ethyl 4-fluoro-6-[2-(2-hydroxypropan-2-yl)azetidin-1-yl]-1-benzofuran-2-carboxylate (rac)

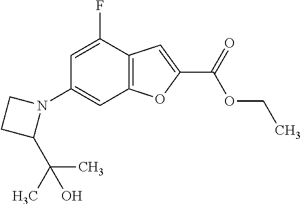 [SMILES: CCOC(=O)c1cc2c(F)cc(N3CCC3C(C)(C)O)cc2o1]

Synthesized analogously to Intermediate 2 using 2-fluoro-6-hydroxy-4-[2-(2-hydroxypropan-2-yl)azetidin-1-yl]benzaldehyde (Intermediate 22, 1.13 g, 4.46 mmol) instead of Intermediate 1 to give 1.47 g of a mixture of ester, acid and not cyclized product as a brown oil which was used without purification.

### Intermediate 24

### 4-Fluoro-6-[2-(2-hydroxypropan-2-yl)azetidin-1-yl]-1-benzofuran-2-carboxylic acid (rac)

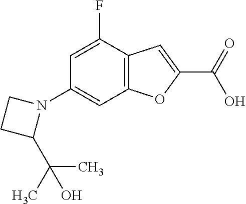 [SMILES: CC(C)(O)C1CCN1c1cc(F)c2cc(C(=O)O)oc2c1]

Synthesized analogously to

Intermediate 3 using ethyl 4-fluoro-6-[2-(2-hydroxypropan-2-yl)azetidin-1-yl]-1-benzofuran-2-carboxylate (rac) (Intermediate 23, 1.47 g, 4.57 mmol) instead of Intermediate 2. The reaction mixture was concentrated in vacuo, the residue was diluted with 30 mL water and acidified to pH 5 with aq. HCl 1 M. Since there was no precipitate the mixture was extracted with dichloromethane/iso-propanol 4/1 twice. The combined organic layers were dried over sodium sulfate and concentrated in vacuo to give 1.07 g (85% purity, 68% yield) of the title compound as a brown solid. ¹H-NMR (400 MHz, DMSO-d₆) δ [ppm]: 1.025 (0.43), 1.040 (0.46), 1.082 (3.04), 1.093 (13.11), 1.194 (16.00), 1.954 (3.42), 2.055 (0.57), 2.061 (0.53), 2.066 (0.58), 2.073 (0.76), 2.078 (0.79), 2.083 (0.86), 2.088 (0.61), 2.094 (0.58), 2.100 (0.57), 2.105 (0.66), 2.259 (0.44), 2.271 (0.70), 2.282 (0.73), 2.293 (0.58), 2.298 (0.60), 2.305 (0.56), 2.309 (0.58), 2.518 (0.43), 2.780 (3.27), 2.939 (4.45), 3.544 (0.51), 3.563 (1.09), 3.567 (0.88), 3.581 (0.92), 3.585 (1.04), 3.604 (0.51), 3.870 (0.76), 3.882 (0.88), 3.891 (1.09), 3.901 (1.06), 3.911 (0.65), 3.923 (0.51), 3.944 (1.18), 3.961 (1.41), 3.966 (1.46), 3.982 (1.11), 4.857 (2.02), 6.766 (1.49), 6.770 (1.75), 6.799 (7.24), 6.828 (0.52), 6.830 (0.47), 6.834 (0.51), 6.836 (0.45), 7.519 (6.12), 7.723 (0.76), 7.729 (0.75); LC-MS (method 1): Rₜ=1.01 min; MS (ESIpos): m/z=294 [M+H]⁺

### Intermediate 25

### N′-(5-Bromo-2-methoxybenzene-1-sulfonyl)-N,N-dimethylmethanimidamide

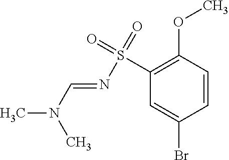

To a stirring solution of 5-bromo-2-methoxybenzene-1-sulfonamide (500 mg, 1.88 mmol) in DMF (5.0 mL) was added 1,1-dimethoxy-N,N-dimethylmethanamine (500 μL, 3.8 mmol; CAS-RN:[4637-24-5]) and the resulting mixture was stirred at room temperature for 1 h. The reaction mixture was concentrated in vacuo to give 605 mg of the title compound as a white solid. ¹H-NMR (500 MHz, DMSO-d₆) δ [ppm]: 2.898 (11.92), 2.899 (11.89), 3.214 (13.75), 3.645 (0.51), 3.840 (16.00), 5.758 (0.93), 7.137 (2.64), 7.154 (2.83), 7.727 (1.58), 7.732 (1.85), 7.745 (1.42), 7.750 (1.73), 7.823 (3.25), 7.828 (3.05), 8.202 (3.10); LC-MS (method 1): Rₜ=0.90 min; MS (ESIpos): m/z=321 [M+H]⁺

### Intermediate 26

### N′-[2-methoxy-5-(4-methylpiperazin-1-yl)benzene-1-sulfonyl]-N,N-dimethylmethanimidamide

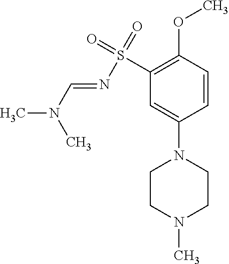 [SMILES: COc1ccc(N2CCN(C)CC2)cc1S(=O)(=O)/N=C/N(C)C]

To a stirring solution of N′-(5-bromo-2-methoxybenzene-1-sulfonyl)-N,N-dimethylmethanimidamide (Intermediate 25, 113 mg, 352 μmol) and 1-methylpiperazine (47 μL, 420 μmol; CAS-RN:[109-01-3]) in 1,4-dioxane (1.5 ml) was added cesium carbonate (287 mg, 880 μmol; CAS-RN:[534-17-8]) and XPhos Pd G2 (27.7 mg, 35.2 μmol; CAS-RN:[1310584-14-5]) subsequently. The suspension was stirred for 1 h at 100° C. The resulting mixture was diluted with 1 mL DMF, filtrated and purified by preparative HPLC (water+0.1% HCOOH/acetonitrile) to give 88.0 mg (73% yield) of the title compound as an off white solid. ¹H-NMR (400 MHz, DMSO-d₆) δ [ppm]: 2.181 (0.52), 2.228 (11.49), 2.243 (0.54), 2.461 (2.70), 2.473 (3.79), 2.518 (2.21), 2.522 (1.33), 2.727 (3.15), 2.881 (12.20), 2.887 (4.86), 3.030 (2.88), 3.043 (3.47), 3.054 (2.78), 3.195 (12.92), 3.511 (4.03), 3.745 (16.00), 7.020 (1.92), 7.043 (2.76), 7.126 (1.38), 7.134 (1.47), 7.149 (0.90), 7.157 (1.02), 7.303 (2.55), 7.310 (2.31), 7.949 (0.49), 8.157 (6.00), 8.169 (3.33); LC-MS (method 1): Rₜ=0.49 min; MS (ESIpos): m/z=341 [M+H]⁺

### Intermediate 27

### 2-Methoxy-5-(4-methylpiperazin-1-yl)benzene-1-sulfonamide

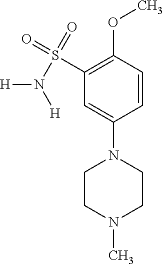 [SMILES: COc1ccc(N2CCN(C)CC2)cc1S(N)(=O)=O]

To a stirring suspension of N′-[2-methoxy-5-(4-methylpiperazin-1-yl)benzene-1-sulfonyl]-N,N-dimethylmethanimidamide (Intermediate 26, 86.0 mg, 253 μmol) in ethanol (1.5 ml) was added ammonium hydroxide (3.0 mL, 25% purity; CAS-RN:[1336-21-6]) and the reaction mixture was stirred at room temperature overnight. The resulting solution was concentrated in vacuo to give 66 mg (92% yield) of the title compound as an off white solid. ¹H-NMR (400 MHz, DMSO-d₆) δ [ppm]: 2.225 (13.10), 2.244 (1.08), 2.327 (0.56), 2.456 (3.98), 2.468 (5.91), 2.481 (6.43), 2.669 (0.55), 3.027 (4.02), 3.039 (5.33), 3.051 (4.21), 3.196 (0.76), 3.512 (5.37), 3.675 (1.12), 3.745 (0.72), 3.818 (16.00), 7.001 (5.29), 7.073 (1.84), 7.096 (3.28), 7.141 (1.74), 7.149 (1.95), 7.164 (1.01), 7.171 (1.19), 7.262 (2.88), 7.270 (2.72), 8.160 (5.15); LC-MS (method 1): Rₜ=0.48 min; MS (ESIpos): m/z=286 [M+H]⁺

### Intermediate 28

### 2,2,5,6-Tetramethyl-2,3-dihydro-1-benzofuran-7-sulfonamide

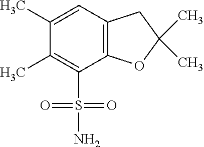

2,2,5,6-tetramethyl-2,3-dihydro-1-benzofuran-7-sulfonyl chloride (5.00 g, 18.2 mmol) was suspended in ammonia in tetrahydrofuran (180 mL, 0.50 M, 91 mmol; CAS-RN:[7664-41-7]) and stirred at room temperature overnight. The reaction mixture was concentrated in vacuo to give a white solid which was suspended in 50 mL water and stirred for 15 min. The undissolved solid was collected by filtration, washed with water and dried in vacuo at 50° C. to give 4.75 g (102% yield) of the title compound as a white solid. ¹H-NMR (400 MHz, DMSO-d₆) δ [ppm]: 1.428 (16.00), 2.168 (6.54), 2.399 (5.49), 2.954 (2.77), 6.973 (1.21), 7.166 (1.68), 7.185 (1.15), 7.311 (1.42), 7.438 (1.13); LC-MS (method 1): Rₜ=1.01 min; MS (ESIpos): m/z=256 [M+H]⁺

### Intermediate 29

### Methyl {[-1-(3-fluoro-4-formyl-5-hydroxyphenyl)azetidin-2-yl]methyl}carbamate (rac)

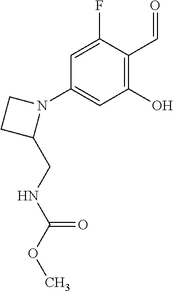 [SMILES: COC(=O)NCC1CCN1c1cc(O)c(C=O)c(F)c1]

Synthesized and purified analogously to Intermediate 1 using Methyl N-[(azetidin-2-yl)methyl]carbamate hydrochloride (1.25 g, 6.92 mmol) instead of azetidine to give 912 mg (56% yield) of the title compound as a yellow solid. ¹H-NMR (400 MHz, DMSO-d₆) δ [ppm]: 2.086 (0.69), 2.099 (0.98), 2.114 (1.18), 2.122 (1.11), 2.136 (0.95), 2.150 (0.54), 2.374 (0.53), 2.388 (0.71), 2.398 (1.16), 2.411 (1.21), 2.419 (1.25), 2.425 (1.03), 2.433 (0.96), 2.437 (1.03), 2.447 (0.56), 2.461 (0.58), 2.523 (1.38), 3.266 (0.57), 3.282 (1.06), 3.301 (1.44), 3.317 (2.48), 3.333 (16.00), 3.374 (1.31), 3.385 (1.59), 3.400 (1.34), 3.410 (0.81), 3.426 (0.77), 3.436 (0.66), 3.461 (0.55), 3.731 (0.96), 3.747 (1.24), 3.754 (2.15), 3.770 (2.18), 3.776 (1.51), 3.792 (1.12), 3.948 (0.90), 3.970 (1.75), 3.983 (1.66), 4.005 (0.66), 4.266 (0.47), 4.279 (1.18), 4.296 (1.48), 4.312 (1.22), 4.325 (0.53), 5.662 (3.21), 5.878 (2.04), 5.912 (2.07), 7.431 (1.08), 7.445 (2.03), 7.460 (1.09), 9.775 (14.60), 11.958 (2.64); LC-MS (method 1): Rₜ=0.97 min; MS (ESIpos): m/z=283 [M+H]+

### Intermediate 30

### Ethyl 4-fluoro-6-[2-[(methoxycarbonylamino)methyl]azetidin-1-yl]benzofuran-2-carboxylat (rac)

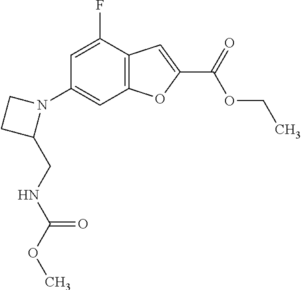 [SMILES: CCOC(=O)c1cc2c(F)cc(N3CCC3CNC(=O)OC)cc2o1]

Synthesized analogously to Intermediate 2 using Methyl {[-1-(3-fluoro-4-formyl-5-hydroxyphenyl)azetidin-2-yl]methyl}carbamate (900 mg, 3.19 mmol) instead of 4-(Azetidin-1-yl)-2-fluoro-6-hydroxybenzaldehyde to give 970 mg (87% yield) of a mixture of ester, acid and not cyclized product as a brown oil which was used without purification. LC-MS (method 1): Rₜ=1.21 min; MS (ESIpos): m/z=351 [M+H]⁺

### Intermediate 31

### 4-Fluoro-6-[2-{[(methoxycarbonyl)amino]methyl}azetidin-1-yl]-1-benzofuran-2-carboxylic acid (rac)

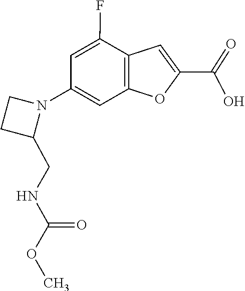 [SMILES: COC(=O)NCC1CCN1c1cc(F)c2cc(C(=O)O)oc2c1]

Synthesized analogously to

Intermediate 3 using ethyl 4-fluoro-6-[-2-{[(methoxycarbonyl)amino]methyl}azetidin-1-yl]-1-benzofuran-2-carboxylate (rac) (Intermediate 30, 970 mg, 2.77 mmol) instead of Azetidine. The reaction mixture was concentrated in vacuo, the residue was diluted with 15 mL water and acidified to pH 5 with aq. HCl 1 M (˜7 ml). Since there was no filtrateable precipitate the mixture was extracted with dichloromethane/iso-propanol 4/1 twice. The combined organic layers were dried over sodium sulfate and concentrated in vacuo to give a brown oil which was purified by preparative HPLC (water+0.1% HCOOH/acetonitrile) to give 250 mg (27% yield) of the title compound as an offwhite solid. ¹H-NMR (400 MHz, DMSO-d₆) δ [ppm]: 2.073 (12.76), 2.083 (9.84), 2.114 (0.52), 2.133 (1.14), 2.140 (1.08), 2.157 (1.75), 2.178 (1.50), 2.197 (0.67), 2.317 (0.76), 2.327 (1.21), 2.339 (1.56), 2.344 (1.46), 2.349 (1.67), 2.355 (1.36), 2.360 (1.44), 2.371 (1.50), 2.376 (1.32), 2.387 (0.71), 2.399 (0.60), 2.518 (1.71), 2.522 (1.05), 3.252 (1.50), 3.268 (2.32), 3.287 (2.77), 3.304 (3.59), 3.319 (2.97), 3.364 (1.97), 3.406 (2.40), 3.417 (2.66), 3.421 (2.59), 3.431 (2.30), 3.440 (1.82), 3.451 (1.75), 3.456 (1.74), 3.467 (1.47), 3.492 (1.17), 3.581 (1.51), 3.600 (3.33), 3.622 (3.23), 3.641 (1.39), 3.730 (0.64), 3.915 (1.33), 3.926 (1.68), 3.936 (2.36), 3.946 (2.41), 3.955 (1.47), 3.967 (1.12), 4.081 (0.70), 4.097 (1.79), 4.112 (2.11), 4.128 (1.70), 4.144 (0.66), 6.377 (3.41), 6.380 (3.41), 6.407 (3.33), 6.409 (3.28), 6.545 (6.64), 7.418 (1.66), 7.433 (3.16), 7.447 (1.64), 7.528 (15.66), 7.531 (16.00); LC-MS (method 1): Rₜ=0.94 min; MS (ESIpos): m/z=323 [M+H]⁺

### Intermediate 32

### Methyl 6-(3-phenylazetidin-1-yl)-1-benzofuran-2-carboxylate

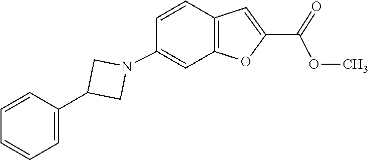 [SMILES: COC(=O)c1cc2ccc(N3CC(c4ccccc4)C3)cc2o1]

Synthesized analogously to Intermediate 4 using 3-phenylazetidine (219 mg, 1.65 mmol) instead of azetidine hydrogen chloride. The crude product was purified by HT-HPLC (acidic method) to give 18.0 mg (4% yield) of the title compound as a white solid. ¹H-NMR (400 MHz, DMSO-d₆) δ [ppm]: 2.077 (0.58), 2.521 (1.35), 2.526 (0.85), 3.845 (16.00), 3.879 (1.65), 3.894 (2.53), 3.913 (2.40), 3.964 (0.42), 3.971 (0.60), 3.988 (0.54), 4.004 (0.78), 4.024 (0.47), 4.325 (1.94), 4.345 (3.31), 4.365 (1.75), 6.588 (1.40), 6.592 (1.45), 6.608 (1.36), 6.614 (1.59), 6.686 (1.89), 7.244 (0.55), 7.256 (0.40), 7.262 (1.23), 7.267 (0.50), 7.275 (0.53), 7.279 (1.02), 7.283 (0.57), 7.346 (1.24), 7.362 (1.12), 7.366 (3.08), 7.379 (0.59), 7.384 (2.95), 7.393 (3.03), 7.396 (3.46), 7.413 (1.29), 7.418 (0.86), 7.573 (2.53), 7.594 (2.42), 7.625 (4.05), 7.628 (3.87); LC-MS (method 1): Rₜ=1.42 min; MS (ESIpos): m/z=308 [M+H]⁺

### Intermediate 33

### 6-(3-Phenylazetidin-1-yl)-1-benzofuran-2-carboxylic acid

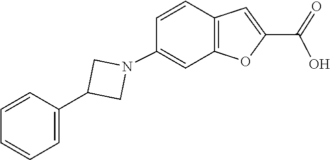 [SMILES: O=C(O)c1cc2ccc(N3CC(c4ccccc4)C3)cc2o1]

Synthesized analogously to Intermediate 7 using Methyl 6-(3-phenylazetidin-1-yl)-1-benzofuran-2-carboxylate (Intermediate 32, 18.0 mg, 58.6 μmol) instead of Methyl 6-(3-fluoroazetidin-1-yl)-1-benzofuran-2-carboxylate to give 15.0 mg (83% yield) of the title compound as a white solid which was used without further purification. LC-MS (method 1): Rₜ=1.22 min; MS (ESIpos): m/z=294 [M+H]⁺

### Intermediate 34

### Methyl 6-(3-ethoxyazetidin-1-yl)-1-benzofuran-2-carboxylate

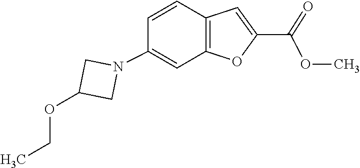 [SMILES: CCOC1CN(c2ccc3cc(C(=O)OC)oc3c2)C1]

Synthesized and purified analogously to Intermediate 32 using 3-ethoxyazetidine (167 mg, 1.65 mmol) instead of 3-phenylazetidine to give 80.0 mg (85% purity, 18% yield) of the title compound as a grey solid. ¹H-NMR (400 MHz, DMSO-d6) δ[ppm]: 1.126 (4.42), 1.144 (9.92), 1.161 (4.53), 2.518 (1.79), 2.522 (1.14), 3.427 (1.40), 3.444 (4.40), 3.461 (4.55), 3.479 (1.34), 3.661 (1.47), 3.672 (1.58), 3.683 (1.65), 3.694 (1.68), 3.836 (16.00), 4.114 (1.50), 4.131 (1.90), 4.152 (1.56), 4.411 (0.68), 4.416 (0.62), 4.422 (0.51), 4.427 (1.11), 4.438 (0.56), 4.442 (0.56), 6.509 (1.43), 6.514 (1.58), 6.531 (1.47), 6.536 (1.72), 6.606 (1.98), 7.128 (0.47), 7.130 (0.47), 7.147 (0.95), 7.164 (0.59), 7.167 (0.58), 7.352 (0.47), 7.355 (0.48), 7.370 (0.47), 7.373 (0.94), 7.376 (0.74), 7.390 (0.59), 7.393 (0.58), 7.464 (1.15), 7.484 (0.77), 7.537 (2.51), 7.558 (2.40), 7.603 (3.76), 7.604 (4.10), 8.095 (0.76), 8.115 (0.73); LC-MS (method 1): Rₜ=1.21 min; MS (ESIpos): m/z=276 [M+H]⁺

### Intermediate 35

### 6-(3-Ethoxyazetidin-1-yl)-1-benzofuran-2-carboxylic acid

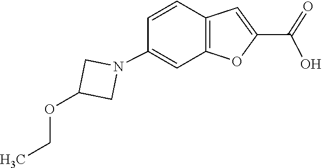 [SMILES: CCOC1CN(c2ccc3cc(C(=O)O)oc3c2)C1]

Synthesized analogously to Intermediate 33 using methyl 6-(3-ethoxyazetidin-1-yl)-1-benzofuran-2-carboxylate (Intermediate 34, 80.0 mg, 291 μmol) instead of methyl 6-(3-phenylazetidin-1-yl)-1-benzofuran-2-carboxylate to give 70.0 mg (80% purity, 74% yield) of the title compound as an ochre solid which was used without purification; LC-MS (method 1): Rₜ=0.98 min; MS (ESIpos): m/z=262 [M+H]⁺

### Intermediate 36

### Methyl 6-(5-azaspiro[2.3]hexan-5-yl)-1-benzofuran-2-carboxylate

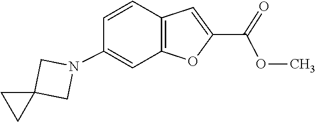 [SMILES: COC(=O)c1cc2ccc(N3CC4(CC4)C3)cc2o1]

Synthesized and purified analogously to Intermediate 32 using 5-azaspiro[2.3]hexane hydrogen chloride (1/1) (197 mg, 1.65 mmol) instead of 3-phenylazetidine to give 40.0 mg (1% yield) of the title compound as a white solid. ¹H-NMR (400 MHz, DMSO-d₆) δ [ppm]: 0.000 (5.21), 0.681 (16.00), 2.521 (0.60), 3.840 (15.85), 3.982 (15.93), 6.514 (1.31), 6.519 (1.42), 6.536 (1.30), 6.541 (1.46), 6.605 (1.75), 7.545 (2.38), 7.567 (2.26), 7.608 (3.46), 7.610 (3.58); LC-MS (method 1): Rₜ=1.32 min; MS (ESIpos): m/z=258 [M+H]⁺

### Intermediate 37

### 6-(5-Azaspiro[2.3]hexan-5-yl)-1-benzofuran-2-carboxylic acid

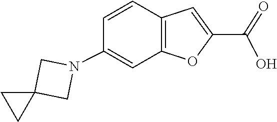 [SMILES: O=C(O)c1cc2ccc(N3CC4(CC4)C3)cc2o1]

Synthesized analogously to Intermediate 33 using methyl 6-(5-azaspiro[2.3]hexan-5-yl)-1-benzofuran-2-carboxylate (Intermediate 36, 40.0 mg, 155 μmol) instead of Methyl 6-(3-phenylazetidin-1-yl)-1-benzofuran-2-carboxylate to give 35.0 mg (88% yield) of the title compound as a white solid which was used without purification, LC-MS (method 1): Rₜ=1.08 min; MS (ESIpos): m/z=244 [M+H]⁺

### Intermediate 38

### Methyl 6-(2-azaspiro[3.3]heptan-2-yl)-1-benzofuran-2-carboxylate

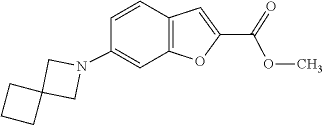 [SMILES: COC(=O)c1cc2ccc(N3CC4(CCC4)C3)cc2o1]

Synthesized and purified analogously to Intermediate 32 using 2-azaspiro[3.3]heptane (160 mg, 1.65 mmol) instead of 3-phenylazetidine to give 90.0 mg (23% yield) of the title compound as a brown solid. ¹H-NMR (400 MHz, DMSO-d₆) δ [ppm]: 1.805 (0.99), 1.825 (1.82), 1.843 (1.22), 1.863 (0.45), 1.987 (0.55), 2.162 (3.81), 2.181 (5.66), 2.200 (3.02), 2.518 (0.60), 3.832 (16.00), 3.853 (14.88), 6.473 (1.23), 6.477 (1.33), 6.494 (1.22), 6.499 (1.43), 6.559 (1.82), 7.516 (2.42), 7.537 (2.28), 7.589 (3.53), 7.591 (3.40); LC-MS (method 1): Rₜ=1.42 min; MS (ESIpos): m/z=272 [M+H]⁺

### Intermediate 39

### 6-(2-Azaspiro[3.3]heptan-2-yl)-1-benzofuran-2-carboxylic acid

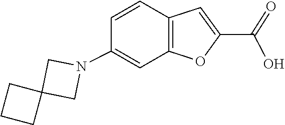 [SMILES: O=C(O)c1cc2ccc(N3CC4(CCC4)C3)cc2o1]

Synthesized analogously to Intermediate 33 using Methyl 6-(2-azaspiro[3.3]heptan-2-yl)-1-benzofuran-2-carboxylate (Intermediate 38, 90.0 mg, 332 μmol) instead of Methyl 6-(3-phenylazetidin-1-yl)-1-benzofuran-2-carboxylate to give 75.0 mg (79% yield) of the title compound as a brown solid which was used without purification, LC-MS (method 1): Rₜ=1.19 min; MS (ESIneg): m/z=256 [M−H]⁻

### Intermediate 40

### Methyl 6-(3,3-dimethylazetidin-1-yl)-1-benzofuran-2-carboxylate

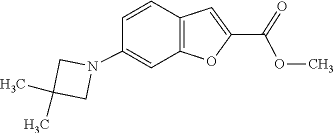

Synthesized and purified analogously to Intermediate 32 using 3,3-dimethylazetidine (140 mg, 1.65 mmol) instead of 3-phenylazetidine to give 40.0 mg (11% yield) of the title compound as an ochre solid. ¹H-NMR (400 MHz, DMSO-d₆) δ [ppm]: 1.293 (16.00), 2.518 (0.54), 3.599 (11.66), 3.832 (10.70), 6.479 (0.95), 6.484 (1.03), 6.500 (0.91), 6.505 (1.04), 6.560 (1.19), 7.517 (1.55), 7.539 (1.48), 7.592 (2.37), 7.594 (2.59); LC-MS (method 1): Rₜ=1.39 min; MS (ESIpos): m/z=260 [M+H]⁺

### Intermediate 41

### 6-(3,3-Dimethylazetidin-1-yl)-1-benzofuran-2-carboxylic acid

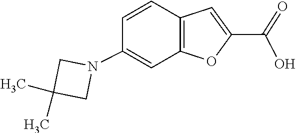 [SMILES: CC1(C)CN(c2ccc3cc(C(=O)O)oc3c2)C1]

Synthesized analogously to Intermediate 33 using methyl 6-(3,3-dimethylazetidin-1-yl)-1-benzofuran-2-carboxylate (40.0 mg, 154 μmol) instead of Methyl 6-(3-phenylazetidin-1-yl)-1-benzofuran-2-carboxylate to give 35.0 mg (88% yield) of the title compound as a light brown solid which was used without purification. LC-MS (method 1): Rₜ=1.16 min; MS (ESIpos): m/z=246 [M+H]⁺

### Intermediate 42

### 6-(2-Oxa-6-azaspiro[3.3]heptan-6-yl)-1-benzofuran-2-carboxylic acid

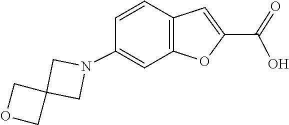 [SMILES: O=C(O)c1cc2ccc(N3CC4(COC4)C3)cc2o1]

To a stirring suspension of 6-bromo-1-benzofuran-2-carboxylic acid (200 mg, 830 μmol), 2-oxa-6-azaspiro[3.3]heptane (98.7 mg, 996 μmol) and cesium carbonate (676 mg, 2.07 mmol; CAS-RN:[534-17-8]) in 1,4-dioxane (3.0 mL) was added XPhos Pd G2 (65.3 mg, 83.0 μmol, 1310584-14-5). The resulting reaction mixture was stirred at 110° C. for 8 h. Water and dichloromethane were added to the reaction mixture and the layers were separated. The aqueous layer was evaporated and the residue purified by HT-HPLC (acidic method) to give 13.0 mg (5% yield) of the title compound as a white solid. ¹H-NMR (400 MHz, DMSO-d₆) δ [ppm]: 2.521 (0.90), 2.526 (0.63), 4.053 (15.69), 4.711 (0.42), 4.731 (16.00), 6.488 (1.40), 6.492 (1.47), 6.509 (1.43), 6.514 (1.58), 6.600 (1.89), 7.480 (3.05), 7.482 (2.98), 7.517 (2.46), 7.538 (2.28); LC-MS (method 3): Rₜ=0.76 min; MS (ESIpos): m/z=260 [M+H]⁺

### Intermediate 43

### 2,3-Difluoro-4-[methyl(propan-2-yl)amino]benzene-1-sulfonamide

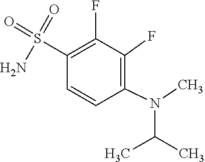 [SMILES: CC(C)N(C)c1ccc(S(N)(=O)=O)c(F)c1F]

To a stirring solution of 2,3,4-Trifluorobenzene-1-sulfonamide (200 mg, 947 μmol) in DMSO (1.5 ml) was added N,N-diisopropylethylamine (250 μl, 1.4 mmol; CAS-RN:[7087-68-5]) and N-methylpropan-2-amine (150 μl, 1.4 mmol; CAS-RN:[4747-21-1]) subsequently. The resulting mixture was stirred at 70° C. overnight. After completion the reaction mixture was filtrated and purified by preparative HPLC (water+0.1% HCOOH/acetonitrile) to give 88.0 mg (35% yield) of the title compound as a white solid. ¹H-NMR (400 MHz, DMSO-d₆) δ [ppm]: 1.130 (16.00), 1.146 (15.90), 2.083 (0.96), 2.518 (1.25), 2.522 (0.76), 2.730 (10.20), 2.734 (10.27), 3.408 (0.52), 3.865 (0.52), 3.882 (1.36), 3.898 (1.84), 3.915 (1.34), 3.931 (0.49), 6.783 (0.72), 6.787 (0.72), 6.807 (1.36), 6.826 (0.84), 6.830 (0.78), 7.358 (0.89), 7.363 (0.83), 7.378 (1.15), 7.381 (1.30), 7.383 (1.32), 7.400 (0.86), 7.405 (0.77), 7.540 (6.21); LC-MS (method 1): Rₜ=1.04 min; MS (ESIpos): m/z=265 [M+H]⁺

### Intermediate 44

### 1-(3-Fluoro-4-formyl-5-hydroxyphenyl)azetidine-2-carboxamide (rac)

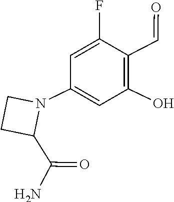 [SMILES: NC(=O)C1CCN1c1cc(O)c(C=O)c(F)c1]

Synthesized analogously to Intermediate 1 using azetidine-2-carboxamide (1.00 g, 9.99 mmol) instead of azetidine and purified by preparative HPLC (water+0.1% HCOOH/acetonitrile) to give 34.0 mg (2% yield) of the title compound as a yellow solid which was used without NMR analytics; LC-MS (method 1): Rₜ=0.74 min; MS (ESIpos): m/z=239 [M+H]⁺

### Intermediate 45

### Ethyl 6-(2-carbamoylazetidin-1-yl)-4-fluoro-benzofuran-2-carboxylate

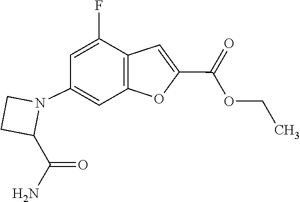 [SMILES: CCOC(=O)c1cc2c(F)cc(N3CCC3C(N)=O)cc2o1]

Synthesized analogously to Intermediate 2 using 1-(3-fluoro-4-formyl-5-hydroxyphenyl)azetidine-2-carboxamide (rac) (35.0 mg, 147 μmol) instead of 4-(Azetidin-1-yl)-2-fluoro-6-hydroxybenzaldehyde to give 28.7 mg (60% yield) of a mixture of ester, acid and not cyclized product as a brown oil which was used without purification.

### Intermediate 46

### 6-(2-Carbamoylazetidin-1-yl)-4-fluoro-1-benzofuran-2-carboxylic acid (rac)

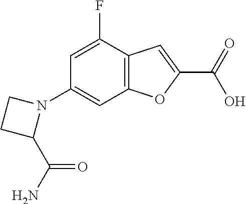 [SMILES: NC(=O)C1CCN1c1cc(F)c2cc(C(=O)O)oc2c1]

Synthesized analogously to

Intermediate 3 using ethyl 6-(2-carbamoylazetidin-1-yl)-4-fluoro-1-benzofuran-2-carboxylate (rac)

Intermediate 45, 25.0 mg, 81.6 μmol) instead of Ethyl 6-(azetidin-1-yl)-4-fluoro-1-benzofuran-2-carboxylate. The reaction mixture was concentrated in vacuo, the residue was acidified to pH 4 and extracted with dichloromethane twice. The combined organic layers were dried over sodium sulfate and concentrated in vacuo to give 8.90 mg (39% yield) of the title compound as a brown oil which was used without purification. LC-MS (method 1): Rₜ=0.72 min; MS (ESIpos): m/z=279 [M+H]⁺

### Intermediate 47

### 4-(2,2-Dimethylazetidin-1-yl)-2-fluoro-6-hydroxy-benzaldehyde

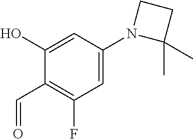 [SMILES: CC1(C)CCN1c1cc(O)c(C=O)c(F)c1]

To a room temperature stirred suspension of 2,4-difluoro-6-hydroxybenzaldehyde (6.00 g, 37.9 mmol, 1.00 eq.) and 2,2-dimethylazetidine (4.85 g, 56.9 mmol, 1.50 eq.) in dimethylsulfoxide (63.3 mL, 0.60 M) was added N,N-Diisopropylethylamine (9.94 mL, 56.9 mmol, 1.5 eq.). The resulting yellow solution was stirred at 60° C. for 3 days and then cooled to room temperature. The mixture was diluted with water (200 mL) and extracted with ethyl acetate (3×100 mL). The combined organic extracts were washed with water (2×200 mL), brine (200 mL), dried (magnesium sulfate), filtered and dry loaded onto Celite. The residue was purified by flash column chromatography (330 g Silica, 0-30% ethyl acetate/hexanes gradient) to give the title compound as a yellow solid (3.27 g). ¹H NMR (400 MHz, Chloroform-d) δ 12.11 (s, 1H), 9.80 (s, 1H), 5.64-5.52 (m, 2H), 3.87-3.78 (m, 2H), 2.24-2.15 (m, 2H), 1.54 (s, 6H); LC-MS (method 5): Rₜ=2.85 min; MS (ESIpos): m/z=224 [M+H]⁺

### Intermediate 48

### Ethyl 6-(2,2-dimethylazetidin-1-yl)-4-fluoro-benzofuran-2-carboxylate

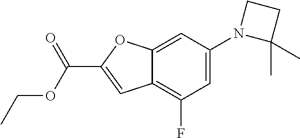 [SMILES: CCOC(=O)c1cc2c(F)cc(N3CCC3(C)C)cc2o1]

To a room temperature stirred solution of 4-(2,2-dimethylazetidin-1-yl)-2-fluoro-6-hydroxy-benzaldehyde (3.27 g, 14.6 mmol, 1.00 eq.) in anhydrous N,N-dimethylformamide (97.6 mL, 0.15 M) was sequentially added potassium carbonate (3.04 g, 22.0 mmol, 1.50 eq.) and ethyl bromoacetate (1.78 mL, 16.1 mmol, 1.10 eq.). The resulting yellow suspension was heated at 160° C. for 2 h and then cooled to room temperature. The dark mixture was diluted with water (100 mL) and extracted with ethyl acetate (3×100 mL). The combined organic extracts were washed with water (100 mL), brine (100 mL), dried (magnesium sulfate), filtered and concentrated under reduced pressure. The residue was purified by flash column chromatography (330 g Silica, 0-30% ethyl acetate/hexanes) to give the title compound as a yellow oil (3.05 g). ¹H NMR (400 MHz, Chloroform-d) δ 7.44 (d, 1H), 6.33 (t, 1H), 6.14 (dd, 1H), 4.40 (q, 2H), 3.77-3.65 (m, 2H), 2.21-2.09 (m, 2H), 1.49 (s, 6H), 1.40 (t, 3H), LC-MS (method 5): Rₜ=3.41 min; MS (ESIpos): m/z=292 [M+H]⁺

### Intermediate 49

### 6-(2,2-Dimethylazetidin-1-yl)-4-fluoro-benzofuran-2-carboxylic acid

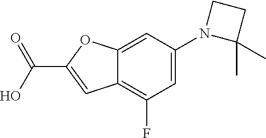 [SMILES: CC1(C)CCN1c1cc(F)c2cc(C(=O)O)oc2c1]

To a room temperature stirred solution of ethyl 6-(2,2-dimethylazetidin-1-yl)-4-fluoro-benzofuran-2-carboxylate (3.04 g, 10.4 mmol, 1.00 eq.) in tetrahydrofuran (34.8 mL, 0.30 M) was added sodium hydroxide (2.00 M in water, 13.0 mL, 26.1 mmol, 2.50 eq.). The resulting mixture was heated at 70° C. for 1 hour and then cooled to room temperature. The mixture was diluted with water (100 mL), acidified to pH 2.0 with hydrochloric acid (1.00 M in water, −27.0 mL) and extracted with ethyl acetate (3×100 mL). The combined organic extracts were washed with brine (100 mL), dried (magnesium sulfate), filtered and concentrated under reduced pressure. The residue was purified by reverse phase flash column chromatography (275 g HP C18, 10-100% acetonitrile/water gradient buffered with 0.1% formic acid) to give the title compound as a yellow solid (2.37 g, >95% purity). ¹H NMR (400 MHz, DMSO-d₆) δ 13.26 (s, 1H), 7.52 (d, 1H), 6.47-6.41 (m, 1H), 6.31 (dd, 1H), 3.71 (t, 2H), 2.11 (t, 2H), 1.47 (s, 6H); LC-MS (method 5): Rₜ=2.67 min; MS (ESIneg): m/z=262 [M−H]⁻

### Intermediate 50

### 2-Fluoro-6-hydroxy-4-(2-methylazetidin-1-yl)benzaldehyde (rac)

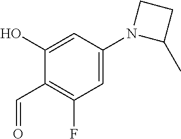

To a room temperature stirred suspension of 2,4-difluoro-6-hydroxybenzaldehyde (4.74 g, 30.0 mmol, 1.00 eq.) and 2-methylazetidine hydrochloride (4.84 g, 45.0 mmol, 1.50 eq.) in dimethylsulfoxide (50.0 mL, 0.60 M) was added N,N-Diisopropylethylamine (15.7 mL, 90.0 mmol, 3.00 eq.). The resulting yellow solution was stirred at 60° C. for 22 h and then cooled to room temperature. The dark solution was diluted with saturated ammonium chloride (100 mL) and extracted with ethyl acetate (3×100 mL). The combined organic extracts were washed with water (2×100 mL), brine (100 mL), dried (magnesium sulfate), filtered and dry loaded onto Celite under reduced pressure. The residue was purified by flash column chromatography (330 g HP silica, 0-30% ethyl acetate/hexanes gradient) followed by reverse phase flash column chromatography (275 g HP C₁₈, 10-100% acetonitrile/water gradient buffered with 0.1% formic acid) to give the title compound as a yellow crystalline solid (1.35 g). ¹H NMR (400 MHz, Chloroform-d) δ 12.10 (s, 1H), 9.82 (d, 1H), 5.65-5.54 (m, 2H), 4.37 (dp, 1H), 4.02 (tdd, 1H), 3.86-3.75 (m, 1H), 2.55 (dddd, 1H), 2.02 (dddd, 1H), 1.48 (d, 3H), LC-MS (method 5): Rₜ=2.72 min; MS (ESIpos): m/z=210 [M+H]⁺

### Intermediate 51

### Ethyl 4-fluoro-6-(2-methylazetidin-1-yl)benzofuran-2-carboxylate

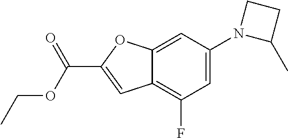 [SMILES: CCOC(=O)c1cc2c(F)cc(N3CCC3C)cc2o1]

To a room temperature stirred solution of 2-fluoro-6-hydroxy-4-(2-methylazetidin-1-yl)benzaldehyde (1.15 g, 5.50 mmol, 1.00 eq.) in anhydrous N,N-dimethylformamide (36.6 mL, 0.15 M) was sequentially added potassium carbonate (1.14 g, 8.24 mmol, 1.50 eq.) and ethyl bromoacetate (669 μL, 6.05 mmol, 1.10 eq.). The resulting yellow suspension was heated at 160° C. for 2 h and then cooled to room temperature. The dark mixture was diluted with acetonitrile, filtered over a Celite plug (washing with acetonitrile) and then concentrated under reduced pressure. The residue was purified by reverse phase flash column chromatography (275 g HP C₁₈, 60-90% acetonitrile/water gradient buffered with 0.1% formic acid) to give the title compound as a brown solid (894 mg). 1H NMR (400 MHz, Chloroform-d) δ 7.45 (d, 1H), 6.35 (dd, 1H), 6.16 (dd, 1H), 4.40 (q, 2H), 4.26-4.13 (m, 1H), 3.97 (ddd, 4.1 Hz, 1H), 3.66 (dt, 1H), 2.46 (dddd, 1H), 2.08 (dddd, 1H), 1.48 (d, 3H), 1.40 (t, 3H); LC-MS (method 5): Rₜ=3.29 min; MS (ESIpos): m/z=278 [M+H]⁺

### Intermediate 52

### 4-Fluoro-6-(2-methylazetidin-1-yl)benzofuran-2-carboxylic acid (rac)

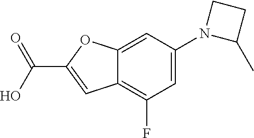 [SMILES: CC1CCN1c1cc(F)c2cc(C(=O)O)oc2c1]

To a room temperature stirred solution of ethyl 4-fluoro-6-(2-methylazetidin-1-yl)benzofuran-2-carboxylate (800 mg, 2.89 mmol, 1.00 eq.) in tetrahydrofuran (9.62 mL, 0.30 M) was added sodium hydroxide (2.00 M in water, 3.61 mL, 7.21 mmol, 2.50 eq.). The resulting mixture was heated at 70° C. for 1 hour and then cooled to room temperature. The mixture was diluted with water (20 mL), acidified to pH 2.0 with hydrochloric acid (1.00 M in water, −7.50 mL) and extracted with ethyl acetate (3×20 mL). The combined organic extracts were washed with brine (20 mL), dried (magnesium sulfate), filtered and concentrated under reduced pressure. The residue was purified by reverse phase flash column chromatography (275 g HP C18, 10-100% acetonitrile/water gradient buffered with 0.1% formic acid) to give the title compound as a yellow solid (692 mg). ¹H NMR (400 MHz, DMSO-d₆) δ 13.28 (s, 1H), 7.53 (d, 1H), 6.49-6.43 (m, 1H), 6.33 (dd, 1H), 4.20 (dt, 1H), 3.98-3.88 (m, 1H), 3.62 (dt, 1H), 2.48-2.36 (m, 1H), 1.99 (ddt, 1H), 1.42 (d, 3H), LC-MS (method 5): Rₜ=2.52 min; MS (ESIpos): m/z=250 [M+H]⁺

### Intermediate 53

### 2-Ethoxy-1-fluoro-3-iodo-benzene

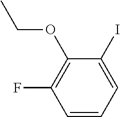

To a stirred solution of 2-fluoro-6-iodo-phenol (1.00 g, 4.20 mmol, 1.00 eq) in anhydrous N,N-dimethylformamide (8.40 mL, 0.50 M) was added potassium carbonate (1.16 g, 8.40 mmol, 2.00 eq) and stirred at room temperature. After five minutes, iodoethane (510 μL, 6.30 mmol, 1.50 eq) was added, and the reaction was left to stir at 70° C. for 20 h. The reaction was washed with water (20 mL), and the aqueous layer was extracted with dichloromethane (3×20 mL). The combined organics washed with brine (20 mL), dried over magnesium sulfate, filtered, and dry loaded onto Celite under reduced pressure. The crude was purified by flash column chromatography (80 g HP Silica, 0-20%, hexane/ethyl acetate) to afford the title compound as a clear oil (1.06 g). 1H NMR (400 MHz, Chloroform-d) δ 7.57-7.49 (m, 1H), 7.12-7.02 (m, 1H), 6.82-6.72 (m, 1H), 4.21-4.11 (m, 2H), 1.46 (t, 3H); LC-MS (method 4): Rₜ=1.65 min; MS (ESIpos): m/z=not detected

### Intermediate 54

### 2-Ethoxy-3-fluoro-benzenesulfonamide

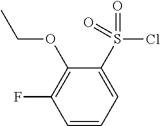

To an oven dried 40 mL vial under an argon atmosphere was added palladium(II) acetate (44.3 mg, 0.20 mmol, 5.00 mol %), CataCXium® A (113 mg, 0.32 mmol, 7.50 mol %), and 1,4-Diazabicyclo[2.2.2]octane bis(sulfur dioxide) adduct (569 mg, 2.37 mmol, 0.60 eq.) were sequentially added argon degassed anhydrous isopropanol (19.7 mL, 0.20 M), 2-ethoxy-1-fluoro-3-iodo-benzene (1.05 g, 3.95 mmol, 1.00 eq.), and triethylamine (1.64 mL, 11.8 mmol, 3.00 eq.). The resulting red solution was heated at 75° C. for 7 d, cooled to room temperature and concentrated under reduced pressure. The residue was resuspended in anhydrous tetrahydrofuran (17.5 mL) and cooled to 0° C. in a ice/water bath. To this mixture was then added sulfuryl chloride (960 μL, 11.8 mmol, 3.00 eq.) dropwise, after which the resulting mixture was warmed to room temperature and stirred for a further 30 minutes. The reaction was quenches with ammonium chloride (20 mL), and the aqueous layer was extracted with dichloromethane (3×20 mL). The combined organics were washed with brine (20 mL), dried over magnesium sulfate, filtered, and dry loaded onto Celite under reduced pressure. The crude was purified by flash column chromatography (80 g HP Silica, 0-20%, ethyl acetate/hexanes) to afford the title compound as an oil (498 mg, >60% purity); LC-MS (method 4): Rt=X min; MS (ESIpos): m/z=not detected.

### Intermediate 55

### 2-Ethoxy-3-fluoro-benzenesulfonamide

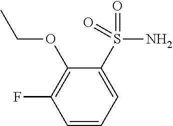

To a stirred solution of 2-ethoxy-3-fluoro-benzenesulfonyl chloride (525 mg, 2.20 mmol, 1.00 eq) in anhydrous tetrahydrofuran (3.67 mL, 0.60 mmol) was added ammonium hydroxide solution (20% in water, 8.57 mL, 44.0 mmol, 20.0 eq) and stirred at room temperature for 30 minutes. The reaction was quenched with hydrochloric acid (1.00 M, 20 mL), and the aqueous layer was extracted with ethyl acetate (3×20 mL). The combined organics were washed with brine (20 mL), dried over magnesium sulfate, filtered, and dry loaded onto Celite under reduced pressure. The crude was purified by flash column chromatography (40 g HP silica, 0-60%, ethyl acetate/hexanes) to afford the title compound as a white solid (192 mg). ¹H NMR (400 MHz, Chloroform-d) δ 7.72-7.64 (m, 1H), 7.37-7.26 (m, 1H), 7.19-7.09 (m, 1H), 5.07 (s, 2H), 4.43-4.32 (m, 2H), 1.50 (t, 3H); LC-MS (method 4): Rₜ=1.25 min; MS (ESIpos): m/z=not detected.

### Intermediate 56

### 2-Iodo-4-sec-butyl-phenol

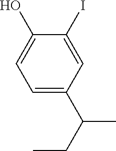

To a stirred solution of 4-sec-butylphenol (5.00 g, 33.3 mmol, 1.00 eq) in acetonitrile (16.6 mL, 2.00 M) was added N-iodosuccinimide (7.49 g, 33.3 mmol, 1.00 eq) and stirred at room temperature for 22 h. Additional N-iodosuccinimide (3.75 g, 16.7 mmol, 0.50 eq) was added and stirred at room temperature for 112 h (five nights). The reaction was quenched with sodium thiosulfate solution (10% in water, 50 mL), and the aqueous layer was extracted with ethyl acetate (3×30 mL). The combined organics were washed with brine (30 mL), dried over magnesium sulfate, filtered, and dry loaded onto Celite under reduced pressure. The crude was purified by flash column chromatography (120 g Silica, 0-30%, ethyl acetate/hexanes) to afford the title compound as an oil (2.35 g). ¹H NMR (400 MHz, Chloroform-d) δ 7.50-7.42 (m, 1H), 7.11-6.99 (m, 1H), 6.95-6.86 (m, 1H), 5.09 (s, 1H), 2.55-2.41 (m, 1H), 1.64-1.46 (m, 2H), 1.33-1.15 (m, 3H), 0.92-0.77 (m, 3H); LC-MS (method 4): Rₜ=1.63 min; MS (ESIneg): m/z=275 [M−H]⁻

### Intermediate 57

### 1-Ethoxy-2-iodo-4-sec-butyl-benzene

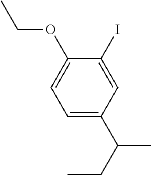

To a stirred solution of 2-iodo-4-sec-butyl-phenol (2.35 g, 8.51 mmol, 1.00 eq) in anhydrous N,N-dimethylformamide (17.0 mL, 0.50 M) was added potassium carbonate (2.35 g, 17.0 mmol, 2.00 eq) and stirred at room temperature. After five minutes, iodoethane (1.03 mL, 12.8 mmol, 1.50 eq) was added, and the reaction was left to stir at 75° C. for 16 h. The reaction was washed with water (30 mL), and the aqueous layer was extracted with dichloromethane (3×30 mL). The combined organics washed with brine (30 mL), dried over magnesium sulfate, filtered, and dry loaded onto Celite under reduced pressure. The crude was purified by flash column chromatography (120 g Silica, 0-20%, hexane/ethyl acetate) to afford the title compound as an oil (1.57 g). ¹H NMR (400 MHz, Chloroform-d) δ 7.61-7.53 (m, 1H), 7.11-7.04 (m, 1H), 6.76-6.69 (m, 1H), 4.12-3.98 (m, 2H), 2.55-2.44 (m, 1H), 1.60-1.42 (m, 5H), 1.23-1.15 (m, 3H), 0.89-0.77 (m, 3H); LC-MS (method 4): Rₜ=1.84 min; MS (ESIpos): m/z=not detected.

### Intermediate 58

### 2-Ethoxy-5-sec-butyl-benzenesulfonyl chloride

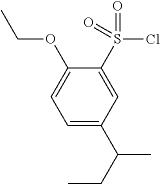 [SMILES: CCOc1ccc(C(C)CC)cc1S(=O)(=O)Cl]

To an oven dried 40 mL vial under an argon atmosphere was added palladium(II) acetate (57.8 mg, 0.26 mmol, 5.00 mol %), CataCXium® A (148 mg, 0.41 mmol, 7.50 mol %), and 1,4-Diazabicyclo[2.2.2]octane bis(sulfur dioxide) adduct (742 mg, 3.09 mmol, 0.60 eq.) were sequentially added argon degassed anhydrous isopropanol (25.7 mL, 0.20 M), 1-ethoxy-2-iodo-4-sec-butyl-benzene (1.57 g, 5.15 mmol, 1.00 eq.), and triethylamine (2.14 mL, 15.4 mmol, 3.00 eq.). The resulting red solution was heated at 75° C. for 16 h, cooled to room temperature and concentrated under reduced pressure. The residue was resuspended in anhydrous tetrahydrofuran (25.7 mL) and cooled to 0° C. in a ice/water bath. To this mixture was then added sulfuryl chloride (1.25 mL, 15.4 mmol, 3.00 eq.) dropwise, after which the resulting mixture was warmed to room temperature and stirred for a further 30 minutes. The reaction was quenches with ammonium chloride (30 mL), and the aqueous layer was extracted with dichloromethane (3×30 mL). The combined organics were washed with brine (30 mL), dried over magnesium sulfate, filtered, and dry loaded onto Celite under reduced pressure. The crude was purified by flash column chromatography (80 g, 0-20%, ethyl acetate/hexanes) to afford the title compound as a black oil (1.17 g). LC-MS (method 4): Rₜ=1.73 min; MS (ESIneg): m/z=not detected

### Intermediate 59

### 2-Ethoxy-5-sec-butyl-benzenesulfonamide

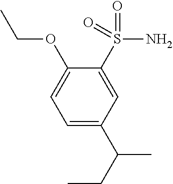 [SMILES: CCOc1ccc(C(C)CC)cc1S(N)(=O)=O]

To a stirred solution of 2-ethoxy-5-sec-butyl-benzenesulfonyl chloride (1.17 g, 4.22 mmol, 1.00 eq) in anhydrous tetrahydrofuran (7.03 mL, 0.60 mmol) was added ammonium hydroxide solution (20% in water, 16.4 mL, 84.3 mmol, 20.0 eq) and stirred at room temperature for 30 minutes. The reaction was quenched with hydrochloric acid (1.00 M, 20 mL), and the aqueous layer was extracted with ethyl acetate (3×20 mL). The combined organics were washed with brine (20 mL), dried over magnesium sulfate, filtered, and dry loaded onto Celite under reduced pressure. The crude was purified by flash column chromatography (80 g HP silica, 0-60%, ethyl acetate/hexanes) to afford the title compound as a clear oil (398 mg). ¹H NMR (400 MHz, Chloroform-d) δ 7.75-7.69 (m, 1H), 7.31 (dd, 1H), 6.95 (d, 1H), 5.03 (s, 2H), 4.28-4.18 (m, 2H), 2.61 (h, 1H), 1.65-1.47 (m, 5H), 1.30-1.18 (m, 3H), 0.85-0.76 (m, 3H); LC-MS (method 4): Rₜ=1.45 min; MS (ESIpos): m/z=258 [M+H]⁺

### Intermediate 60

### 2-Ethoxy-4-fluoro-1-iodo-benzene

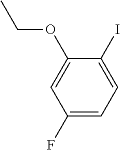

To a stirred solution of 5-fluoro-2-iodo-phenol (1.00 g, 4.20 mmol, 1.00 eq) in anhydrous N,N-dimethylformamide (8.40 mL, 0.50 M) was added potassium carbonate (1.16 g, 8.40 mmol, 2.00 eq) and stirred at room temperature. After five minutes, iodoethane (510 μL, 6.30 mmol, 1.50 eq) was added, and the reaction was left to stir at 70° C. for 16 h. The reaction was washed with water (30 mL), and the aqueous layer was extracted with dichloromethane (3×20 mL). The combined organics were washed with brine (20 mL), dried over magnesium sulfate, filtered, and dry loaded onto Celite under reduced pressure. The crude was purified by flash column chromatrography (80 g, 0-20%, ethyl acetate/hexanes) to afford the title compound as a clear oil (931 mg). 1H NMR (400 MHz, Chloroform-d) δ 7.69 (dd, 1H), 6.55 (dd, 1H), 6.53-6.43 (m, 1H), 4.12-4.00 (m, 2H), 1.49 (t, 3H); LC-MS (method 4): Rₜ=1.65 min; MS (ESIpos): m/z=not detected

### Intermediate 61

### 2-ethoxy-4-fluoro-benzenesulfonyl chloride

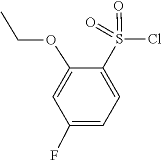

To an oven dried 40 mL vial under an argon atmosphere was added palladium(II) acetate (39.3 mg, 0.17 mmol, 5.00 mol %), CataCXium® A (100 mg, 0.28 mmol, 7.50 mol %), and 1,4-Diazabicyclo[2.2.2]octane bis(sulfur dioxide) adduct (505 mg, 2.10 mmol, 0.60 eq.) were sequentially added argon degassed anhydrous isopropanol (17.5 mL, 0.20 M), 2-ethoxy-4-fluoro-1-iodo-benzene (931 g, 3.50 mmol, 1.00 eq.), and triethylamine (1.46 mL, 10.5 mmol, 3.00 eq.). The resulting red solution was heated at 75° C. for 16 h, cooled to room temperature and concentrated under reduced pressure. The residue was resuspended in anhydrous tetrahydrofuran (17.5 mL) and cooled to 0° C. in a ice/water bath. To this mixture was then added sulfuryl chloride (1.46 mL, 10.5 mmol, 3.00 eq.) dropwise, after which the resulting mixture was warmed to room temperature and stirred for a further 30 minutes. The reaction was quenches with ammonium chloride (20 mL), and the aqueous layer was extracted with dichloromethane (3×20 mL). The combined organics were washed with brine (20 mL), dried over magnesium sulfate, filtered, and dry loaded onto Celite under reduced pressure. The crude was purified by flash column chromatography (80 g, 0-20%, ethyl acetate/hexanes) to afford the title compound as an oil (498 mg). 1H NMR (400 MHz, Chloroform-d) δ 8.02-7.93 (m, 1H), 6.83-6.72 (m, 2H), 4.26 (q, 2H), 1.59-1.53 (m, 3H); LC-MS (method 4): Rₜ=1.21 min; MS (ESIneg): m/z=not detected

### Intermediate 62

### 2-Ethoxy-4-fluoro-benzenesulfonamide

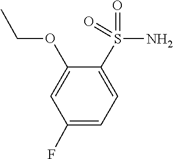

To a stirred solution of 2-ethoxy-4-fluoro-benzenesulfonyl chloride (498 mg, 2.09 mmol, 1.00 eq) in anhydrous tetrahydrofuran (3.48 mL, 0.60 mmol) was added ammonium hydroxide solution (20% in water, 8.13 mL, 41.7 mmol, 20.0 eq) and stirred at room temperature for 30 minutes. The reaction was quenched with hydrochloric acid (1.00 M, 10 mL), and the aqueous layer was extracted with ethyl acetate (3×10 mL). The combined organics were washed with brine (20 mL), dried over magnesium sulfate, filtered, and dry loaded onto Celite under reduced pressure. The crude was purified by flash column chromatography (24 g HP Silica, 0-60%, ethyl acetate/hexanes) to afford the title compound as a white solid (336 mg). ¹H NMR (400 MHz, Chloroform-d) δ 7.96-7.88 (m, 1H), 6.80-6.70 (m, 2H), 4.97 (s, 2H), 4.23 (q, 2H), 1.58-1.50 (m, 3H); LC-MS (method 4): Rₜ=1.20 min; MS (ESIneg): m/z=218 [M−H]⁻

### Intermediate 63

### 1-Ethoxy-2-iodo-4-(trifluoromethyl)benzene

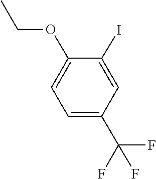

To a stirred solution of 2-iodo-4-(trifluoromethyl)phenol (1.00 g, 3.47 mmol, 1.00 eq) in anhydrous N,N-dimethylformamide (6.94 mL, 0.50 M) was added potassium carbonate (960 mg, 6.94 mmol, 2.00 eq) and stirred at room temperature. After five minutes, iodoethane (420 μL, 5.21 mmol, 1.50 eq) was added, and the reaction was left to stir at 70° C. for 16 h. The reaction was washed with water (30 mL), and the aqueous layer was extracted with dichloromethane (3×20 mL). The combined organic layers were washed with brine (20 mL), dried over magnesium sulfate, filtered, and dry loaded onto Celite under reduced pressure. The residue was purified by flash column chromatrography (80 g, 0-20%, ethyl acetate/hexanes) to afford the title compound as a clear oil (886 mg). ¹H NMR (400 MHz, Chloroform-d) δ 8.04-7.99 (m, 1H), 7.59-7.51 (m, 1H), 6.82 (d, 1H), 4.14 (q, 2H), 1.55-1.47 (m, 3H); LC-MS (method 4): Rₜ=1.73 min; MS (ESIpos): m/z=not detected.

### Intermediate 64

### 2-Ethoxy-5-(trifluoromethyl)benzenesulfonyl chloride

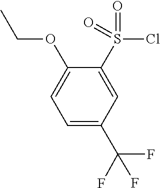 [SMILES: CCOc1ccc(C(F)(F)F)cc1S(=O)(=O)Cl]

To an oven dried 40 mL vial under an argon atmosphere was added palladium(II) acetate (31.5 mg, 0.14 mmol, 5.00 mol %), CataCXium® A (80.4 mg, 0.22 mmol, 7.50 mol %), and 1,4-Diazabicyclo[2.2.2]octane bis(sulfur dioxide) adduct (404 mg, 1.68 mmol, 0.60 eq.) were sequentially added argon degassed anhydrous isopropanol (14.0 mL, 0.20 M), 1-ethoxy-2-iodo-4-(trifluoromethyl)benzene (886 g, 2.80 mmol, 1.00 eq.), and triethylamine (1.17 mL, 8.41 mmol, 3.00 eq.). The resulting red solution was heated at 75° C. for 16 h, cooled to room temperature and concentrated under reduced pressure. The residue was resuspended in anhydrous tetrahydrofuran (14.0 mL) and cooled to 0° C. in a ice/water bath. To this mixture was then added sulfuryl chloride (1.67 mL, 8.41 mmol, 3.00 eq.) dropwise, after which the resulting mixture was warmed to room temperature and stirred for a further 30 minutes. The reaction was quenched with ammonium chloride (20 mL), and the aqueous layer was extracted with dichloromethane (3×20 mL). The combined organics were washed with brine (20 mL), dried over magnesium sulfate, filtered, and dry loaded onto Celite under reduced pressure. The crude was purified by flash column chromatography (40 g HP Silica, 0-20%, ethyl acetate/hexanes) to afford the title compound as an oil (175 mg). ¹H NMR (400 MHz, Chloroform-d) δ 8.26-8.21 (m, 1H), 7.94-7.86 (m, 1H), 7.20 (d, 1H), 4.36 (q, 2H), 1.63-1.54 (m, 3H); LC-MS (method 4): Rₜ=1.63 min; MS (ESIneg): m/z=not detected

### Intermediate 65

### 2-ethoxy-5-(trifluoromethyl)benzenesulfonamide

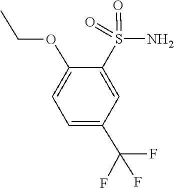 [SMILES: CCOc1ccc(C(F)(F)F)cc1S(N)(=O)=O]

To a stirred solution of 2-ethoxy-5-(trifluoromethyl)benzenesulfonyl chloride (175 mg, 0.60 mmol, 1.00 eq) in anhydrous tetrahydrofuran (1.00 mL, 0.60 mmol) was added ammonium hydroxide solution (20% in water, 2.32 mL, 11.9 mmol, 20.0 eq) and stirred at room temperature for 30 minutes. The reaction was quenched with hydrochloric acid (1.00 M, 10 mL), and the aqueous layer was extracted with ethyl acetate (3×10 mL). The combined organics were washed with brine (10 mL), dried over magnesium sulfate, filtered, and dry loaded onto Celite under reduced pressure. The crude was purified by flash column chromatography (12 g HP Silica, 0-60%, ethyl acetate/hexanes) to afford the title compound as a white solid (45 mg). 1H NMR (400 MHz, Chloroform-d) δ 8.24-8.18 (m, 1H), 7.82-7.75 (m, 1H), 7.16-7.09 (m, 1H), 5.03 (s, 2H), 4.33 (q, 2H), 1.61-1.52 (m, 3H); LC-MS (method 4): Rₜ=1.31 min; MS (ESIneg): m/z=268 [M−H]⁻

### Intermediate 66

### 1-Ethoxy-4-fluoro-2-iodo-benzene

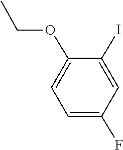

To a stirred solution of 4-fluoro-2-iodo-phenol (1.00 g, 4.20 mmol, 1.00 eq) in anhydrous N,N-dimethylformamide (8.40 mL, 0.50 M) was added potassium carbonate (1.16 g, 8.40 mmol, 2.00 eq) and stirred at room temperature. After five minutes, iodoethane (510 μL, 6.30 mmol, 1.50 eq) was added, and the reaction was left to stir at 70° C. for 17 h. The reaction was washed with water (20 mL), and the aqueous layer was extracted with dichloromethane (3×20 mL). The combined organics washed with brine (20 mL), dried over magnesium sulfate, filtered, and dry loaded onto Celite under reduced pressure. The crude was purified by flash column chromatography (80 g HP Silica, 0-20%, hexanes/ethyl acetate) to afford the title compound as a clear oil (1.02 g). ¹H NMR (400 MHz, Chloroform-d) δ 7.50 (dd, 1H), 7.05-6.95 (m, 1H), 6.73 (dd, 1H), 4.04 (q, 2H), 1.46 (t, 3H); LC-MS (method 4): Rₜ=1.65 min; MS (ESIpos): m/z=not detected

### Intermediate 67

### 2-Ethoxy-5-fluoro-benzenesulfonyl chloride

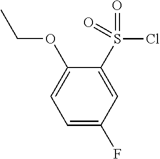

To an oven dried 40 mL vial under an argon atmosphere was added palladium(II) acetate (43.2 mg, 0.19 mmol, 5.00 mol %), CataCXium® A (110 mg, 0.31 mmol, 7.50 mol %), and 1,4-Diazabicyclo[2.2.2]octane bis(sulfur dioxide) adduct (554 mg, 2.31 mmol, 0.60 eq.) were sequentially added argon degassed anhydrous isopropanol (19.2 mL, 0.20 M), 1-ethoxy-4-fluoro-2-iodo-benzene (1023 g, 3.85 mmol, 1.00 eq.), and triethylamine (1.60 mL, 11.5 mmol, 3.00 eq.). The resulting red solution was heated at 75° C. for 16 h, cooled to room temperature and concentrated under reduced pressure. The residue was resuspended in anhydrous tetrahydrofuran (19.2 mL) and cooled to 0° C. in a ice/water bath. To this mixture was then added sulfuryl chloride (930 μL, 11.5 mmol, 3.00 eq.) dropwise, after which the resulting mixture was warmed to room temperature and stirred for a further 30 minutes. The reaction was quenches with ammonium chloride (20 mL), and the aqueous layer was extracted with dichloromethane (3×20 mL). The combined organics were washed with brine (20 mL), dried over magnesium sulfate, filtered, and dry loaded onto Celite under reduced pressure. The crude was purified by flash column chromatography (80 g HP Silica, 0-20%, ethyl acetate/hexanes) to afford the title compound as an oil (295 mg). ¹H NMR (400 MHz, Chloroform-d) δ 7.69 (dd, 1H), 7.43-7.33 (m, 1H), 7.07 (dd, 1H), 4.26 (q, 2H), 1.59-1.49 (m, 3H); LC-MS (method 4): Rₜ=1.55 min; MS (ESIneg): m/z=not detected

### Intermediate 68

### 2-Ethoxy-5-fluoro-benzenesulfonamide

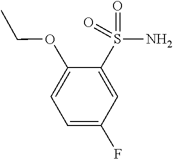

To a stirred solution of 2-ethoxy-5-fluoro-benzenesulfonyl chloride (295 mg, 1.22 mmol, 1.00 eq) in anhydrous tetrahydrofuran (2.03 mL, 0.60 mmol) was added ammonium hydroxide solution (20% in water, 4.73 mL, 24.3 mmol, 20.0 eq) and stirred at room temperature for 30 minutes. The reaction was quenched with hydrochloric acid (1.00 M, 10 mL), and the aqueous layer was extracted with ethyl acetate (3×10 mL). The combined organics were washed with brine (10 mL), dried over magnesium sulfate, filtered, and dry loaded onto Celite under reduced pressure. The crude was purified by flash column chromatography (12 g HP Silica, 0-60%, ethyl acetate/hexanes) to afford the title compound as a white solid (233 mg). 1H NMR (400 MHz, Chloroform-d) δ 7.65 (dd, 1H), 7.22 (ddd, 1H), 7.04-6.95 (m, 1H), 5.05 (s, 2H), 4.23 (q, 2H), 1.54-1.48 (m, 2H); LC-MS (method 4): Rₜ=1.21 min; MS (ESIneg): m/z=218 [M−H]⁻

### Intermediate 69

### 1-(Methoxymethoxy)-4-phenoxy-benzene

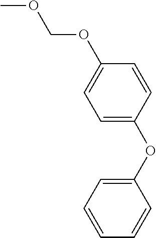 [SMILES: COCOc1ccc(Oc2ccccc2)cc1]

To a 0° C. stirred solution of 4-phenoxyphenol (7.27 g, 39.0 mmol, 1.00 eq.) in anhydrous dichloromethane (130 mL, 0.30 M) was added chloromethyl methyl ether (4.45 mL, 58.6 mmol, 1.50 eq.) dropwise. The resulting solution was warmed to room temperature and stirred for 24 h, after which the mixture was diluted with water (200 mL) and extracted with dichloromethane (3×100 mL). The combined organic extracts were washed with hydrochloric acid (1.00 M in water, 200 mL), sodium hydroxide (1.00 M in water, 200 mL), brine (200 mL), dried (magnesium sulfate), filtered, and dry loaded onto Celite under reduced pressure. The residue was purified by flash column chromatography (330 g Silica, 0-20% ethyl acetate/hexanes gradient) to give the title compound as a colourless oil (6.76 g). 1H NMR (400 MHz, Chloroform-d) δ 7.34-7.27 (m, 2H), 7.08-6.93 (m, 7H), 5.15 (s, 2H), 3.50 (s, 3H); LC-MS (method 5): Rₜ=2.99 min; MS (ESIneg): m/z=229 [M−H]⁻

### Intermediate 70

### 2-Iodo-1-(methoxymethoxy)-4-phenoxy-benzene

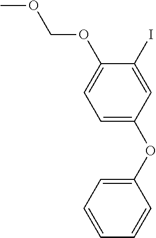 [SMILES: COCOc1ccc(Oc2ccccc2)cc1I]

To a −78° C. stirred solution of 1-(methoxymethoxy)-4-phenoxy-benzene (6.76 g, 29.4 mmol, 1.00 eq.) in anhydrous tetrahydrofuran (147 mL, 0.20 M) was added n-butyllithium solution (2.50 M in hexanes, 13.5 mL, 33.8 mmol, 1.15 eq.) dropwise, after which the yellow solution was warmed to room temperature and stirred for 30 minutes. The mixture was then cooled back to −78° C. and a solution of iodine (8.64 g, 34.1 mmol, 1.16 eq.) in anhydrous tetrahydrofuran (169 mL, 0.20 M) was added dropwise via cannula. After complete addition, the mixture was warmed to room temperature and stirred for 30 minutes. The mixture was then quenched with sodium thiosulfate (10 wt. % in water, 200 mL), partially concentrated under reduced pressure and extracted with diethyl ether (3×200 mL). The combined organic extracts were washed with brine (200 mL), dried (magnesium sulfate), filtered and concentrated under reduced pressure. The residue was purified by flash reverse phase column chromatography (275 g HP C₁₈, 40-100% acetonitrile/water buffered with 0.1% formic acid) to give the title compound as a colourless oil (6.68 g). 1H NMR (400 MHz, Chloroform-d) δ 7.46 (d, 1H), 7.39-7.27 (m, 2H), 7.14-7.01 (m, 2H), 7.04-6.92 (m, 3H), 5.20 (s, 2H), 3.54 (s, 3H); LC-MS (method 5): Rₜ=3.34 min; MS (ESIpos): m/z=not found.

### Intermediate 71

### 2-iodo-4-phenoxy-phenol

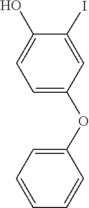 [SMILES: Oc1ccc(Oc2ccccc2)cc1I]

To a stirred solution of sodium iodide (5.62 g, 37.5 mmol, 2.00 eq) and 2-iodo-1-(methoxymethoxy)-4-phenoxy-benzene (6.68 g, 18.8 mmol, 1.00 eq) in anhydrous acetonitrile (94.0 mL, 0.20 M) was added trimethylsilyl chloride (3.55 mL, 28.1 mmol, 1.50 eq) dropwise at 0° C. and stirred for 10 minutes then warmed to room temperature. After 3 h, the reaction was quenched with aqueous sodium thiosulfate (10% w/w, 50 mL), and the aqueous layer was extracted with diethyl ether (3×30 mL). The combined organics were dried over sodium sulfate, filtered, and dry loaded onto Celite under reduced pressure. The crude was purified using flash column chromatography (120 g, 0-30%, hexane/ethyl acetate) to afford the title compound as an orange oil (5.53 g). ¹H NMR (400 MHz, Chloroform-d) δ 7.37-7.27 (m, 3H), 7.13-7.03 (m, 1H), 7.00-6.91 (m, 4H), 5.12 (s, 1H); LC-MS (method 5): Rₜ=2.87 min; MS (ESIneg): m/z=310 [M−H]⁻

### Intermediate 72 1-Ethoxy-2-iodo-4-phenoxy-benzene

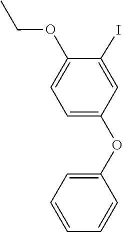 [SMILES: CCOc1ccc(Oc2ccccc2)cc1I]

To a stirred solution of 2-iodo-4-phenoxy-phenol (1.50 g, 4.81 mmol, 1.00 eq) in anhydrous N,N-dimethylformamide (9.61 mL, 0.50 M) was added potassium carbonate (1.33 g, 9.61 mmol, 2.00 eq) and stirred at room temperature. After five minutes, iodoethane (410 μL, 5.29 mmol, 1.10 eq) was added and stirred at 100° C. for 16 h. The reaction was washed with water (30 mL), and the aqueous layer was extracted with dichloromethane (3×30 mL). The combined organic layers were washed with brine (30 mL), dried over magnesium sulfate, filtered, and dry loaded onto Celite under reduced pressure. The crude was purified using flash column chromatography (80 g HP Silica, 0-20%, hexane/ethyl acetate) to afford the title compound as an oil (1.49 g). 1H NMR (400 MHz, Chloroform-d) δ 7.47 (d, 1H), 7.36-7.27 (m, 2H), 7.12-7.03 (m, 1H), 7.03-6.90 (m, 3H), 6.81-6.74 (m, 1H), 4.07 (q, 2H), 1.48 (t, 3H); LC-MS (method 4): Rₜ=1.79 min; MS (ESIpos): m/z=not detected

### Intermediate 73

### 2-Ethoxy-5-phenoxy-benzenesulfonyl chloride

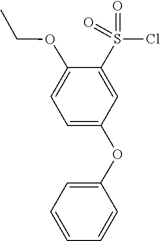 [SMILES: CCOc1ccc(Oc2ccccc2)cc1S(=O)(=O)Cl]

To an oven dried 40 mM vial under an argon atmosphere was added palladium(II) acetate (49.2 mg, 0.22 mmol, 5.00 mol %), CataCXium® A (126 mg, 0.35 mmol, 7.50 mol %), and 1,4-Diazabicyclo[2.2.2]octane bis(sulfur dioxide) adduct (632 mg, 2.63 mmol, 0.60 eq.) were sequentially added argon degassed anhydrous isopropanol (21.9 mL, 0.20 M), 1-ethoxy-2-iodo-4-phenoxy-benzene (1.49 g, 4.38 mmol, 1.00 eq.), and triethylamine (1.82 mL, 13.2 mmol, 3.00 eq.). The resulting red solution was heated at 75° C. for 16 h, cooled to room temperature and concentrated under reduced pressure. The residue was resuspended in anhydrous tetrahydrofuran (21.9 mL) and cooled to 0° C. in a ice/water bath. To this mixture was then added sulfuryl chloride (1.06 mL, 13.2 mmol, 3.00 eq.) dropwise, after which the resulting mixture was warmed to room temperature and stirred for a further 30 minutes. The mixture was diluted with saturated ammonium chloride (30 mL) and extracted with dichloromethane (3×30 mL). The combined organic extracts were dried over magnesium sulfate, filtered, and dry loaded onto Celite under reduced pressure. The residue was purified by flash column chromatography (80 g HP Silica, 0-20% ethyl acetate/hexanes gradient) to give the title compound as an off white solid (687 mg). 1H NMR (400 MHz, Chloroform-d) δ 7.62 (d, 1H), 7.40-7.29 (m, 3H), 7.14 (t, 1H), 7.07 (d, 1H), 7.01-6.93 (m, 2H), 4.27 (q, 2H), 1.56-1.50 (m, 3H); LC-MS (method 4): Rₜ=1.69 min; MS (ESIpos): m/z=not detected

### Intermediate 74

### 2-ethoxy-5-phenoxy-benzenesulfonamide

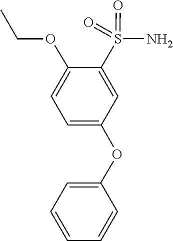 [SMILES: CCOc1ccc(Oc2ccccc2)cc1S(N)(=O)=O]

To a stirred solution of 2-ethoxy-5-phenoxy-benzenesulfonyl chloride (684 mg, 2.19 mmol, 1.00 eq) in anhydrous tetrahydrofuran (2.64 mL, 0.6 M) was added ammonium hydroxide (20% in water, 8.52 mL, 43.7 mmol, 20.0 eq) and stirred at room temperature for 30 minutes. The reaction was washed with hydrochloric acid (1.00 M, 15 mL), and the aqueous layer was extracted with ethyl acetate (3×20 mL). The combined organic layers were washed with brine (20 mL), dried over magnesium sulfate, filtered, and dry loaded onto Celite under reduced pressure. The crude was purified by flash column chromatography (40 g HP Silica, 0-60%, ethyl acetate/hexane) to give the title compound as a white solid (590 mg). ¹H NMR (400 MHz, Chloroform-d) δ 7.60 (d, 1H), 7.38-7.28 (m, 2H), 7.24-7.16 (m, 1H), 7.15-7.06 (m, 1H), 7.04-6.99 (m, 1H), 6.96 (dd, 2H), 5.04 (s, 2H), 4.24 (q, 2H), 1.57-1.48 (m, 3H); LC-MS (method 4): Rₜ=1.42 min; MS (ESIpos): m/z=294 [M+1]⁺

### Intermediate 75 2-iodo-4-(trifluoromethoxy)phenol

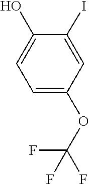 [SMILES: Oc1ccc(OC(F)(F)F)cc1I]

To a stirred solution of 4-(trifluoromethoxy)phenol (4.90 g, 27.5 mmol, 1.00 eq) in acetonitrile (13.8 mL, 2.00 M) was added p-toluenesulfonic acid monohydrate (4.74 g, 27.5 mmol, 1.00 eq) at room temperature. After ten minutes, N-iodosuccinimide (6.19 g, 27.5 mmol, 1.00 eq) was added to the reaction mixture. The reaction stirred for 6 h before being quenched with aqueous sodium sulfite (10% w/w, 30 mL). The mixture was acidified using aqueous hydrochloric acid (1.00 M, 30 mL), and the aqueous layer was extracted with ethyl acetate (3×30 mL). The combined organic layers were dried over magnesium sulfate, filtered, and dry loaded onto Celite under reduced pressure. The crude was purified by flash column chromatography (120 g HP Silica, 0-20%, ethyl acetate/hexanes) to afford the desired product as an orange oil (6.03 g): ¹H NMR (400 MHz, Chloroform-d) δ 7.56-7.50 (m, 1H), 7.18-7.10 (m, 1H), 7.03-6.95 (m, 1H), 5.40-5.29 (m, 1H); LC-MS (method 4): Rₜ=1.75 min; MS (ESIpos): m/z=303 [M−H]⁻

### Intermediate 76

### 1-Ethoxy-2-iodo-4-(trifluoromethoxy)benzene

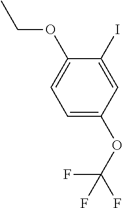

To a solution of 2-iodo-4-(trifluoromethoxy)phenol (1.50 g, 4.93 mmol, 1.00 eq) in anhydrous N,N-dimethylformamide (9.87 mL, 0.50 M) was added potassium carbonate (1.36 g, 9.87 mmol, 2.00 eq) and stirred at room temperature. After five minutes, iodoethane (440 μL, 5.43 mmol, 1.10 eq) was added, and the reaction stirred for 16 h at 70° C. The reaction was washed with water (20 mL), and the aqueous was extracted with dichloromethane (3×20 mL). The combined organic layers were washed with brine (20 mL), dried over magnesium sulfate, filtered, and dry loaded onto Celite under reduced pressure. The crude was purified using flash column chromatography (80 g HP Silica, 0-30%, hexane/ethyl acetate) to afford the title compound as an oil (922 mg). ¹H NMR (400 MHz, Chloroform-d) δ 7.68-7.62 (m, 1H), 7.21-7.13 (m, 1H), 6.80-6.72 (m, 1H), 4.08 (q, 2H), 1.48 (t, 3H); LC-MS (method 4): Rₜ=1.75 min; MS (ESIpos): m/z=not detected

### Intermediate 77

### 2-Ethoxy-5-(trifluoromethoxy)benzenesulfonyl chloride

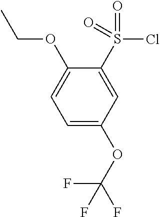 [SMILES: CCOc1ccc(OC(F)(F)F)cc1S(=O)(=O)Cl]

To an oven dried 40 mL vial under an argon atmosphere were added palladium(II) acetate (31.4 mg, 0.14 mmol, 5.00 mol %), CataCXium® A (75.1 mg, 0.21 mmol, 7.50 mol %), and 1,4-Diazabicyclo[2.2.2]octane bis(sulfur dioxide) adduct (403 mg, 1.68 mmol, 0.60 eq.) were sequentially added argon degassed anhydrous isopropanol (14.0 mL, 0.20 M), 1-benzyloxy-2-iodo-4-isopropyl-benzene (928 mg, 2.79 mmol, 1.00 eq.), and triethylamine (1.17 mL, 8.38 mmol, 3.00 eq.). The resulting red solution was heated at 75° C. for 16 h, cooled to room temperature and concentrated under reduced pressure. The residue was resuspended in anhydrous tetrahydrofuran (14.0 mL) and cooled to 0° C. in a ice/water bath. To this mixture was then added sulfuryl chloride (680 μL, 8.38 mmol, 3.00 eq.) dropwise, after which the resulting mixture was warmed to room temperature and stirred for a further 30 min. The mixture was diluted with saturated ammonium chloride (30 mL) and extracted with dichloromethane (3×30 mL). The combined organic extracts were dried over magnesium sulfate, filtered, and dry loaded onto Celite under reduced pressure. The residue was purified by flash column chromatography (80 g HP Silica, 0-20% ethyl acetate/hexanes gradient) to give the title compound as a white solid (192 mg). ¹H NMR (400 MHz, Chloroform-d) δ 7.84 (d, 1H), 7.57-7.48 (m, 1H), 7.15-7.08 (m, 1H), 4.30 (q, 2H), 1.56 (t, 3H); LC-MS (method 4): Rₜ=1.64 min; MS (ESIpos): m/z=not detected

### Intermediate 78

### 2-Ethoxy-5-(trifluoromethoxy)benzenesulfonamide

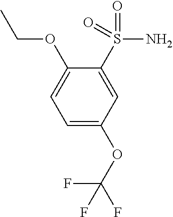 [SMILES: CCOc1ccc(OC(F)(F)F)cc1S(N)(=O)=O]

To a room temperature stirred solution of 2-ethoxy-5-(trifluoromethoxy)benzenesulfonyl chloride (192 mg, 0.63 mmol, 1.00 eq.) in anhydrous tetrahydrofuran (1.05 mL, 0.60 M) was added ammonium hydroxide solution (20% in water, 2.45 mL, 12.6 mmol, 20.0 eq.). The resulting suspension was stirred at room temperature for 30 minutes and then diluted with hydrochloric acid (1.00 M in water, 10 mL). The mixture was extracted with ethyl acetate (3×10 mL) and the combined organic extracts were washed with brine (10 mL), dried over magnesium sulfate, filtered, and dry loaded onto Celite. The residue was purified by flash column chromatography (12 g HP Silica, 0-60% ethyl acetate/hexanes gradient) to give the title compound as a white solid (154 mg). 1H NMR (400 MHz, Chloroform-d) δ 7.82 (d, 1H), 7.43-7.35 (m, 1H), 7.04 (d, 1H), 5.04 (s, 2H), 4.27 (q, 2H), 1.58-1.50 (m, 3H); LC-MS (method 5): Rₜ=1.37 min; MS (ESIneg): m/z=284 [M−H]⁻

### Intermediate 79

### 2-iodo-4-isopropyl-phenol

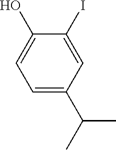

To a stirred solution of 4-isopropylphenol (10.0 g, 73.4 mmol, 1.00 eq) in acetonitrile (36.7 mL) was added p-toluenesulfonic acid monohydrate (12.6 g, 36.7 mmol, 1.00 eq) at room temperature. After twenty minutes, N-iodosuccinimide (16.5 g, 73.43 mmol, 1.00 eq) was added to the reaction mixture. The reaction stirred for 45 minutes before being quenched with aqueous sodium sulfite (10% w/w). The mixture was acidified using aqueous hydrochloric acid (1.00 M), and the aqueous layer was extracted ethyl acetate (3×50 mL). The combined organic layers were dried over magnesium sulfate, filtered, and dry loaded onto Celite under reduced pressure. The crude was purified by flash column chromatography (330 g silica, 0-20% ethyl acetate/hexanes) to afford the desired product as an orange oil. ¹H NMR (400 MHz, Chloroform-d) δ 7.50 (d, 1H), 7.10 (dd, 1H), 6.92 (d, 1H), 5.12 (s, 1H), 2.82 (hept, 1H), 1.21 (d, 7H); LC-MS (method 5): Rₜ=2.90 min; MS (ESIneg): m/z=261 [M−H]⁻

### Intermediate 80

### 1-Benzyloxy-2-iodo-4-isopropyl-benzene

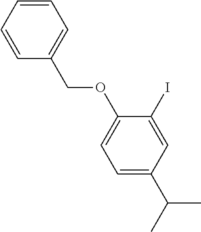 [SMILES: CC(C)c1ccc(OCc2ccccc2)c(I)c1]

To a stirred solution of 2-iodo-4-isopropyl-phenol (1.00 g, 3.82 mmol, 1.00 eq) in anhydrous N,N-dimethylformamide (7.63 mL, 0.50 M) was added potassium carbonate (1.05 g, 7.63 mmol, 2.00 eq), and the reaction stirred for 5 minutes at room temperature. Benzyl bromide (540 μL, 4.58 mmol, 1.20 eq) was added, and the reaction was left stirring at 100° C. for 7 h. The reaction was cooled to room temperature, the mixture was washed with water (20 mL), and the aqueous layer was extracted with dichloromethane (3×20 mL). The combined organic layers were washed with brine (20 mL), dried over magnesium sulfate, filtered, and dry loaded onto Celite under reduced pressure. The crude was purified by flash column chromatography (80 g HP Silica, 0-10% ethyl acetate/hexanes) to afford the desired product as a clear oil (1.182 g). 1H NMR (400 MHz, Chloroform-d) δ 7.66 (d, 1H), 7.54-7.46 (m, 2H), 7.44-7.35 (m, 2H), 7.35-7.28 (m, 1H), 7.12 (dd, 1H), 6.82-6.75 (m, 1H), 5.13 (s, 2H), 2.82 (hept, 1H), 1.24-1.16 (m, 6H); LC-MS (method 5): Rₜ=3.80 min; MS (ESIpos): m/z=not detected

### Intermediate 81

### 2-benzyloxy-5-isopropyl-benzenesulfonyl chloride

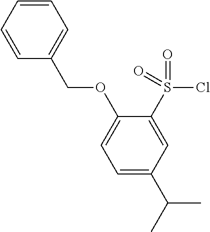 [SMILES: CC(C)c1ccc(OCc2ccccc2)c(S(=O)(=O)Cl)c1]

To a flame dried 40 mL vial under an argon atmosphere was added palladium(II) acetate (17.2 mg, 0.08 mmol, 5.00 mol %), CataCXium® A (41.2 mg, 0.11 mmol, 7.50 mol %), and 1,4-diazabicyclo[2.2.2]octane bis(sulfur dioxide) adduct (221 mg, 0.92 mmol, 0.60 eq.) was sequentially added argon degassed anhydrous isopropanol (7.67 mL, 0.20 M), 1-benzyloxy-2-iodo-4-isopropyl-benzene (540 mg, 1.53 mmol, 1.00 eq.), and triethylamine (0.64 mL, 4.60 mmol, 3.00 eq.). The resulting red solution was heated at 75° C. for 18 h, cooled to room temperature and concentrated under reduced pressure. The residue was resuspended in anhydrous tetrahydrofuran (7.67 mL) and cooled to 0° C. in a ice/water bath. To this mixture was then added sulfuryl chloride (0.37 mL, 4.60 mmol, 3.00 eq.) dropwise, after which the resulting mixture was warmed to room temperature and stirred for a further 30 minutes. The mixture was diluted with saturated ammonium chloride (50 mL) and extracted with dichloromethane (3×20 mL). The combined organic extracts were dried (magnesium sulfate), filtered and dry loaded onto Celite under reduced pressure. The residue was purified by flash column chromatography (24 g HP Silica, 0-20% ethyl acetate/hexanes gradient) to give the title compound as an off white solid (345 mg). ¹H NMR (400 MHz, Chloroform-d) δ 7.82 (d, 1H), 7.55-7.50 (m, 2H), 7.48 (dd, 1H), 7.44-7.37 (m, 2H), 7.37-7.30 (m, 1H), 7.06 (d, 1H), 5.33 (s, 2H), 2.93 (hept, 1H), 1.25 (d, 6H); LC-MS (method 5): Rₜ=3.48 min; MS (ESIpos): m/z=not detected.

### Intermediate 82

### 2-Benzyloxy-5-isopropyl-benzenesulfonamide

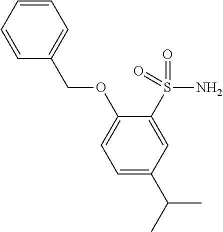 [SMILES: CC(C)c1ccc(OCc2ccccc2)c(S(N)(=O)=O)c1]

To a room temperature stirred solution of 2-benzyloxy-5-isopropyl-benzenesulfonyl chloride (340 mg, 1.05 mmol, 1.00 eq.) in anhydrous tetrahydrofuran (1.74 mL, 0.60 M) was added ammonium hydroxide solution (28% in water, 2.91 mL, 20.9 mmol, 20.0 eq.). The resulting suspension was stirred at room temperature for 30 minutes and then diluted with hydrochloric acid (1.00 M in water, 20 mL). The mixture was extracted with ethyl acetate (3×20 mL) and the combined organic extracts were washed with brine (20 mL), dired (magnesium sulfate) filtered and dry loaded onto Celite. The residue was purified by flash column chromatography (40 g HP Silica, 0-60% ethyl acetate/hexanes gradient) to give the title compound as a colourless oil (305 mg). 1H NMR (400 MHz, Chloroform-d) δ 7.80 (d, 1H), 7.53-7.46 (m, 2H), 7.48-7.32 (m, 4H), 7.03 (d, 1H), 5.23 (s, 2H), 4.95 (s, 2H), 2.92 (hept, 1H), 1.24 (d, 6H); LC-MS (method 5): Rₜ=2.73 min; MS (ESIneg): m/z=304 [M−H]⁻

### Intermediate 83

### 1-(Cyclopropylmethoxy)-2-iodo-4-isopropyl-benzene

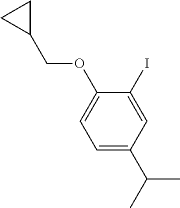

To a stirred solution of 2-iodo-4-isopropyl-phenol (1.31 g, 4.99 mmol, 1.00 eq) in anhydrous N,N-dimethylformamide (9.98 mL) was added potassium carbonate (1.38 g, 9.98 mmol, 2.00 eq), and the reaction stirred for 5 minutes at room temperature. (Iodomethyl)cyclopropane (0.510 mL, 5.49 mmol, 1.10 eq) was added, and the reaction was left stirring at 100° C. for 20 h. The reaction was cooled to room temperature, the mixture was washed with water (20 mL), and the aqueous layer was extracted with methylene chloride (3×20 mL). The combined organic layers were washed with brine (20 mL), dried over magnesium sulfate, filtered, and dry loaded onto Celite under reduced pressure. The crude was purified by flash column chromatography (80 g silica, 0-10% ethyl acetate/hexanes) to afford the desired product as a clear oil (606 mg). ¹H NMR (400 MHz, Chloroform-d) δ 7.63 (d, 1H), 7.11 (dd, 1H), 6.73 (d, 1H), 3.86 (d, 2H), 2.81 (hept, 1H), 1.35-1.24 (m, 1H), 1.21 (d, 6H), 0.68-0.56 (m, 2H), 0.47-0.34 (m, 2H); LC-MS (method 4): Rₜ=1.76 min; MS (ESIpos): m/z=not detected

### Intermediate 84

### 2-(Cyclopropylmethoxy)-5-isopropyl-benzenesulfonyl chloride

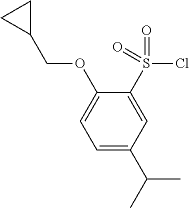 [SMILES: CC(C)c1ccc(OCC2CC2)c(S(=O)(=O)Cl)c1]

To an oven dried 40 mL vial under an argon atmosphere was added palladium(II) acetate (21.3 mg, 0.0900 mmol, 5.00 mol %), CataCXium® A (51.0 mg, 0.140 mmol, 7.50 mol %), and 1,4-diazabicyclo[2.2.2]octane bis(sulfur dioxide) adduct (274 mg, 1.14 mmol, 0.600 eq.) were sequentially added argon degassed anhydrous isopropanol (9.49 mL, 0.20 M), 1-(cyclopropylmethoxy)-2-iodo-4-isopropyl-benzene (600 mg, 1.90 mmol, 1.00 eq.), and triethylamine (790 μL, 5.69 mmol, 3.00 eq.). The resulting red solution was heated at 75° C. for 15 h, cooled to room temperature and concentrated under reduced pressure. The residue was resuspended in anhydrous tetrahydrofuran (9.49 mL) and cooled to 0° C. in a ice/water bath. To this mixture was then added sulfuryl chloride (460 μL, 5.69 mmol, 3.00 eq.) dropwise, after which the resulting mixture was warmed to room temperature and stirred for a further 30 minutes. The mixture was diluted with saturated ammonium chloride (20 mL) and extracted with dichloromethane (3×20 mL). The combined organic extracts were dried over magnesium sulfate, filtered, and dry loaded onto Celite under reduced pressure. The residue was purified by flash column chromatography (40 g HP Silica, 0-20% ethyl acetate/hexanes gradient) to give the title compound as a white solid (425 mg). ¹H NMR (400 MHz, Chloroform-d) δ 7.81-7.76 (m, 1H), 7.52-7.45 (m, 1H), 7.04-6.97 (m, 1H), 4.07 (d, 2H), 3.01-2.85 (m, 1H), 1.42-1.29 (m, 1H), 1.29-1.22 (m, 6H), 0.74-0.61 (m, 2H), 0.47 (dt, 2H); LC-MS (method 4): Rₜ=1.73 min; MS (ESIpos): m/z=not detected

### Intermediate 85

### 2-(Cyclopropylmethoxy)-5-isopropyl-benzenesulfonamide

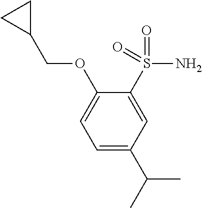 [SMILES: CC(C)c1ccc(OCC2CC2)c(S(N)(=O)=O)c1]

To a room temperature stirred solution of 2-(cyclopropylmethoxy)-5-isopropyl-benzenesulfonyl chloride (425 mg, 1.47 mmol, 1.00 eq.) in anhydrous tetrahydrofuran (2.45 mL, 0.60 M) was added ammonium hydroxide solution (20% in water, 5.73 mL, 29.4 mmol, 20.0 eq.). The resulting suspension was stirred at room temperature for 30 minutes and then diluted with hydrochloric acid (1.00 M in water, 20 mL). The mixture was extracted with ethyl acetate (3×20 mL) and the combined organic extracts were washed with brine (20 mL), dried over magnesium sulfate, filtered, and dry loaded onto Celite. The residue was purified by flash column chromatography (40 g HP Silica, 0-60% ethyl acetate/hexanes gradient) to give the title compound as a white solid (335 mg). ¹H NMR (400 MHz, Chloroform-d) δ 7.77 (d, 1H), 7.35 (dd, 1H), 6.96-6.88 (m, 1H), 5.10 (s, 2H), 3.98 (d, 2H), 2.91 (hept, 1H), 1.45-1.30 (m, 1H), 1.30-1.20 (m, 6H), 0.76-0.63 (m, 2H), 0.46-0.32 (m, 2H); LC-MS (method 4): Rₜ=1.46 min; MS (ESIneg): m/z=268 [M−H]⁻

### Intermediate 86

### 2-iodo-4-isopropyl-1-(2,2,2-trifluoroethoxy)benzene

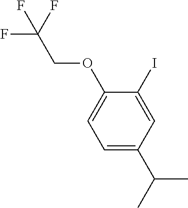 [SMILES: CC(C)c1ccc(OCC(F)(F)F)c(I)c1]

To a stirred solution of 2-iodo-4-isopropyl-phenol (1.13 g, 4.33 mmol, 1.00 eq) in anhydrous N,N-dimethylformamide (8.66 mL) was added potassium carbonate (1.20 g, 8.66 mmol, 2.00 eq), and the reaction stirred for 5 minutes at room temperature. 1,1,1-trifluoro-2-iodoethane (0.470 mL, 4.76 mmol, 1.10 eq) was added, and the reaction was left stirring at 100° C. for 20 h. The reaction was cooled to room temperature, the mixture was washed with water (20 mL), and the aqueous layer was extracted with methylene chloride (3×20 mL). The combined organic layers were washed with brine (20 mL), dried over magnesium sulfate, filtered, and dry loaded onto Celite under reduced pressure. The crude was purified by flash column chromatography (80 g, 0-10% ethyl acetate/hexanes) to afford the desired product as a clear oil (647 mg). 1H NMR (400 MHz, Chloroform-d) δ 7.66 (d, 1H), 7.16 (dd, 1H), 6.79 (d, 1H), 4.36 (q, 2H), 2.84 (hept, 1H), 1.22 (d, 6H), LC-MS (method 4): Rₜ=1.76 min; MS (ESIpos): m/z=not detected.

### Intermediate 87

### 5-Isopropyl-2-(2,2,2-trifluoroethoxy)benzenesulfonyl chloride

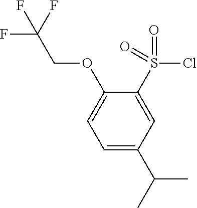 [SMILES: CC(C)c1ccc(OCC(F)(F)F)c(S(=O)(=O)Cl)c1]

To a flame dried 40 mL vial under an argon atmosphere was added palladium(II) acetate (21.1 mg, 0.09 mmol, 5.00 mol %), CataCXium® A (50.6 mg, 0.14 mmol, 7.50 mol %), and 1,4-diazabicyclo[2.2.2]octane bis(sulfur dioxide) adduct (271 mg, 1.13 mmol, 0.600 eq.) were sequentially added argon degassed anhydrous isopropanol (9.40 mL, 0.20 M), 2-iodo-4-isopropyl-1-(2,2,2-trifluoroethoxy)benzene (647 mg, 1.88 mmol, 1.00 eq.), and triethylamine (790 μL, 5.64 mmol, 3.00 eq.). The resulting red solution was heated at 75° C. for 18 h, cooled to room temperature and concentrated under reduced pressure. The residue was resuspended in anhydrous tetrahydrofuran (9.40 mL) and cooled to 0° C. in a ice/water bath. To this mixture was then added sulfuryl chloride (460 μL, 5.64 mmol, 3.00 eq.) dropwise, after which the resulting mixture was warmed to room temperature and stirred for a further 30 minutes. The mixture was diluted with saturated ammonium chloride (20 mL) and extracted with dichloromethane (3×20 mL). The combined organic extracts were dried over magnesium sulfate, filtered, and dry loaded onto Celite under reduced pressure. The residue was purified by flash column chromatography (40 g HP Silica, 0-20% ethyl acetate/hexanes gradient) to give the title compound as an off white solid (334 mg). 1H NMR (400 MHz, Chloroform-d) δ 7.85 (d, 1H), 7.60-7.52 (m, 1H), 7.08-7.01 (m, 1H), 4.55 (q, 2H), 2.98 (hept1H), 1.32-1.23 (m, 6H); LC-MS (method 4): Rₜ=1.62 min; MS (ESIpos): m/z=not detected

### Intermediate 88

### 5-Isopropyl-2-(2,2,2-trifluoroethoxy)benzenesulfonamide

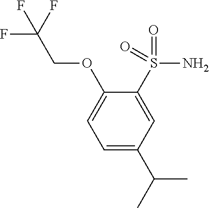 [SMILES: CC(C)c1ccc(OCC(F)(F)F)c(S(N)(=O)=O)c1]

To a room temperature stirred solution of 5-isopropyl-2-(2,2,2-trifluoroethoxy)benzenesulfonyl chloride (330 mg, 1.04 mmol, 1.00 eq.) in anhydrous tetrahydrofuran (1.74 mL, 0.60 M) was added ammonium hydroxide solution (20% in water, 4.06 mL, 20.8 mmol, 20.0 eq.). The resulting suspension was stirred at room temperature for 30 minutes and then diluted with hydrochloric acid (1.00 M in water, 20 mL). The mixture was extracted with ethyl acetate (3×20 mL) and the combined organic extracts were washed with brine (20 mL), dried over magnesium sulfate, filtered, and dry loaded onto Celite under reduced pressure. The residue was purified by flash column chromatography (24 g HP Silica, 0-60% ethyl acetate/hexanes gradient) to give the title compound as a white solid (195 mg). 1H NMR (400 MHz, Chloroform-d) δ 7.82 (d, 1H), 7.45-7.38 (m, 1H), 7.02-6.95 (m, 1H), 4.96 (s, 2H), 4.52 (q, 2H), 3.03-2.87 (m, 1H), 1.30-1.22 (m, 6H); LC-MS (method 4): Rₜ=1.42 min; MS (ESIneg): m/z=296 [M−H]⁻

### Intermediate 89

### 4-(tert-butyl)-1-cyclopropoxy-2-iodobenzene

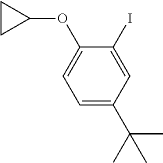

To a stirred solution of 4-tert-butyl-2-iodo-phenol (2.00 g, 7.24 mmol, 1.00 eq.) in anhdyrous N,N-dimethylacetamide (24.1 mL, 0.30 M) was sequentially added cesium carbonate (4.72 g, 14.5 mmol, 2.00 eq.) and bromocyclopropane (1.45 mL, 18.1 mmol, 2.50 eq.). The resulting mixture was heated at 150° C. for 24 h and then cooled to room temperature. The reaction mixture was diluted with water (30 mL), and extracted with ethyl acetate (3×30 mL). The combined organic extracts were washed with brine (30 15 mL), dried (magnesium sulfate), filtered, and dry loaded onto Celite under reduced pressure. The residue was purified by flash column chromatography (120 g Silica, 0-20%, ethyl acetate/hexanes gradient) to give the title compound as an orange oil (819 mg). ¹H NMR (400 MHz, Chloroform-d) δ 7.74 (d, 1H), 7.32 (dd, 1H), 7.13-7.06 (m, 1H), 3.83-3.74 (m, 1H), 1.31-1.26 (m, 9H), 0.91-0.74 (m, 4H); LC-MS (method 4): Rₜ=1.79 min; MS (ESIpos): m/z=not detected

### Intermediate 90

### 5-(tert-butyl)-2-cyclopropoxybenzenesulfonamide

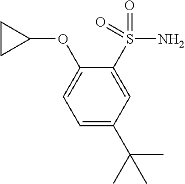 [SMILES: CC(C)(C)c1ccc(OC2CC2)c(S(N)(=O)=O)c1]

To a 40 mL vial was added 4-tert-butyl-1-(cyclopropoxy)-2-iodo-benzene (819 mg, 2.59 mmol, 1.00 eq), palladium(II) acetate (29.1 mg, 0.13 mmol, 5.00 mol %), CataCXium® A (74.3 mg, 0.21 mmol, 7.50 mol %), and 1,4-Diazabicyclo[2.2.2]octane bis(sulfur dioxide) adduct (373 mg, 1.55 mmol, 0.60 eq.) sequentially followed by the addition of degassed anhydrous isopropanol (13.0 mL, 0.20 M) and triethylamine (1.08 mL, 7.77 mmol, 3.00 eq.). The resulting red solution was heated at 75° C. for 17 h, cooled to room temperature and concentrated under reduced pressure. The residue was resuspended in anhydrous tetrahydrofuran (13.0 mL) and cooled to 0° C. in a ice/water bath. To this mixture was then added sulfuryl chloride (630 μL, 7.77 mmol, 3.00 eq.) dropwise, after which the resulting mixture was warmed to room temperature and stirred for a further 30 minutes. The reaction was quenched with ammonium chloride (20 mL), and the aqueous layer was extracted with dichloromethane (3×20 mL). The combined organics were washed with brine (20 mL), dried over magnesium sulfate, filtered, and dry loaded onto Celite under reduced pressure. The crude was purified by flash column chromatography (40 g HP Silica, 0-20%, ethyl acetate/hexanes) to afford 5-tert-butyl-2-(cyclopropoxy)benzenesulfonyl chloride as an oil, which was suspended in anhydrous tetrahydrofuran (13.0 mL). To this solution was added ammonium hydroxide solution (28.0-30.0% NH₃ basis, 7.37 mL, 51.8 mmol, 20.0 eq.) and stirred at room temperature for 30 minutes. The reaction was quenched with aqueous hydrochloric acid (1.00 M, 20 mL) and extracted with ethyl acetate (3×20 mL). The combined organics were washed with brine (20 mL), dried over magnesium sulfate, filtered, and dry loaded onto Celite under reduced pressure. The crude was purified by flash column chromatography (40 g HP Silica, 0-60%, ethyl acetate/hexanes) to afford the title compound as a white solid (162 mg). ¹H NMR (400 MHz, Chloroform-d) δ 7.91 (d, 1H), 7.55 (dd, 1H), 7.36-7.29 (m, 1H), 4.93 (s, 2H), 3.98-3.88 (m, 1H), 1.32 (s, 9H), 0.96-0.80 (m, 4H), LC-MS (method 4): Rₜ=1.41 min; MS (ESIpos): m/z=270 [M+H]⁺

### Intermediate 91

### 1-(Benzyloxy)-4-(tert-butyl)-2-iodobenzene

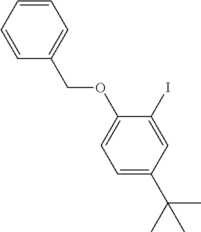 [SMILES: CC(C)(C)c1ccc(OCc2ccccc2)c(I)c1]

To a stirred suspension of 4-tert-butyl-2-iodo-phenol (1.50 g, 5.43 mmol, 1.00 eq.) and potassium carbonate (1.50 g, 10.9 mmol, 2.00 eq.) in anhydrous N,N-dimethylformamide (2.72 mL, 2.00 M) was added benzyl bromide (1.29 mL, 10.9 mmol, 2.00 eq.). The resulting brown suspension was heated at 60° C. for 48 h and then cooled to room temperature. The mixture was diluted with water (50 mL) and extracted with dichloromethane (3×50 mL). The combined organic extracts were washed with brine (50 mL), dried (magnesium sulfate), filtered and dry loaded onto Celite. The residue was purified by reverse phase flash column chromatography (275 g HP C₁₈, 60-100% acetonitrile/water gradient buffered with 0.1% formic acid) to give the title compound as a colourless oil (1.42 g). ¹H NMR (400 MHz, Chloroform-d) δ 7.79 (d, 1H), 7.50 (ddt, 2H), 7.44-7.35 (m, 2H), 7.37-7.24 (m, 2H), 6.79 (d, 1H), 5.13 (s, 2H), 1.28 (s, 9H); LC-MS (method 5): Rₜ=3.77 min; MS (ESIneg): m/z=365 [M−H]⁻

### Intermediate 92

### 2-(Benzyloxy)-5-(tert-butyl)benzenesulfonamide

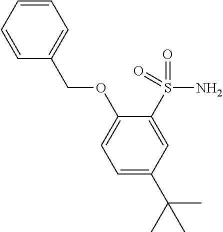 [SMILES: CC(C)(C)c1ccc(OCc2ccccc2)c(S(N)(=O)=O)c1]

To a 40 mL vial under an argon atmosphere was added 1-benzyloxy-4-tert-butyl-2-iodo-benzene (1.43 g, 3.89 mmol, 1.00 eq), palladium(II) acetate (43.7 mg, 0.19 mmol, 5.00 mol %), CataCXium® A (112 mg, 0.31 mmol, 7.50 mol %), and 1,4-Diazabicyclo[2.2.2]octane bis(sulfur dioxide) adduct (561 mg, 2.33 mmol, 0.60 eq.) followed by the addition of degassed anhydrous isopropanol (19.5 mL, 0.20 M) and triethylamine (1.62 mL, 11.7 mmol, 3.00 eq.). The resulting red solution was heated at 75° C. for 16 h, cooled to room temperature and concentrated under reduced pressure. The residue was resuspended in anhydrous tetrahydrofuran (19.5 mL) and cooled to 0° C. in a ice/water bath. To this mixture was then added sulfuryl chloride (0.94 mL, 11.7 mmol, 3.00 eq.) dropwise, after which the resulting mixture was warmed to room temperature and stirred for a further 30 minutes. The reaction was quenched with ammonium chloride (30 mL), and the aqueous layer was extracted with dichloromethane (3×30 mL). The combined organics were washed with brine (30 mL), dried (magnesium sulfate), filtered, and dry loaded onto Celite under reduced pressure. The crude was purified by flash column chromatography (80 g HP Silica, 0-20%, ethyl acetate/hexanes) to afford 2-benzyloxy-5-tert-butyl-benzenesulfonyl chloride as an oil, which was suspended in anhydrous tetrahydrofuran (19.5 mL). To this solution was added ammonium hydroxide solution (28.0-30.0% NH₃ basis, 11.1 mL, 76.8 mmol, 20.0 eq.) and stirred at room temperature for 30 minutes. The reaction was quenched with aqueous hydrochloric acid (1.00 M, 30 mL) and extracted with ethyl acetate (3×30 mL). The combined organics were washed with brine (30 mL), dried over magnesium sulfate, filtered, and dry loaded onto Celite under reduced pressure. The crude was purified by flash column chromatography (80 g HP Silica, 0-60%, ethyl acetate/hexanes) to afford the title compound as a white solid (457 mg). ¹H NMR (400 MHz, Chloroform-d) δ 7.98-7.92 (m, 1H), 7.57-7.46 (m, 3H), 7.46-7.32 (m, 3H), 7.07-7.00 (m, 1H), 5.23 (s, 2H), 4.95 (s, 2H), 1.32 (s, 9H), LC-MS (method 4): Rₜ=1.50 min; MS (ESIpos): m/z=320 [M+H]⁺

### Intermediate 93

### 4-(tert-butyl)-1-(cyclopropylmethoxy)-2-iodobenzene

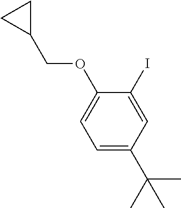 [SMILES: CC(C)(C)c1ccc(OCC2CC2)c(I)c1]

To a stirred suspension of 4-tert-butyl-2-iodo-phenol (1.50 g, 5.43 mmol, 1.00 eq.) and potassium carbonate (1.50 g, 10.9 mmol, 2.00 eq.) in anhydrous N,N-dimethylformamide (2.72 mL, 2.00 M) was added bromomethylcyclopropane (1.01 mL, 10.9 mmol, 2.00 eq.). The resulting brown suspension was heated at 60° C. for 48 h and then cooled to room temperature. The mixture was diluted with water (50 mL) and extracted with dichloromethane (3×50 mL). The combined organic extracts were washed with brine (50 mL), dried (magnesium sulfate), filtered and dry loaded onto Celite. The residue was purified by flash column chromatography (80 g HP Silica, 0-20% ethyl acetate/hexanes gradient) to give the title compound as a colourless oil (1.27 g). 1H NMR (400 MHz, Chloroform-d) δ 7.76 (d, 1H), 7.31-7.24 (m, 1H), 6.73 (d, 1H), 3.86 (d, 2H), 1.37-1.22 (m, 1H), 1.28 (s, 9H), 0.69-0.56 (m, 2H), 0.47-0.34 (m, 2H); LC-MS (method 5): Rₜ=3.72 min; MS (ESIpos): m/z=not detected

### Intermediate 94

### 5-(tert-butyl)-2-(cyclopropylmethoxy)benzenesulfonamide

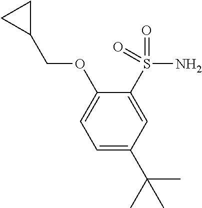 [SMILES: CC(C)(C)c1ccc(OCC2CC2)c(S(N)(=O)=O)c1]

To a 40 mL vial under an argon atmosphere was added 4-tert-butyl-1-(cyclopropylmethoxy)-2-iodo-benzene (1.27 g, 3.84 mmol, 1.00 eq), palladium(II) acetate (43.1 mg, 0.19 mmol, 5.00 mol %), CataCXium® A (110 mg, 0.31 mmol, 7.50 mol %), and 1,4-Diazabicyclo[2.2.2]octane bis(sulfur dioxide) adduct (554 mg, 2.30 mmol, 0.60 eq.) followed by the addition of degassed anhydrous isopropanol (19.2 mL, 0.20 M) and triethylamine (1.60 mL, 11.5 mmol, 3.00 eq.). The resulting red solution was heated at 75° C. for 16 h, cooled to room temperature and concentrated under reduced pressure. The residue was resuspended in anhydrous tetrahydrofuran (19.2 mL) and cooled to 0° C. in a ice/water bath. To this mixture was then added sulfuryl chloride (0.93 mL, 11.5 mmol, 3.00 eq.) dropwise, after which the resulting mixture was warmed to room temperature and stirred for a further 30 minutes. The reaction was quenched with ammonium chloride (30 mL), and the aqueous layer was extracted with dichloromethane (3×30 mL). The combined organics were washed with brine (30 mL), dried (magnesium sulfate), filtered, and dry loaded onto Celite under reduced pressure. The crude was purified by flash column chromatography (80 g HP silica, 0-20%, ethyl acetate/hexanes gradient) to give 5-tert-butyl-2-(cyclopropylmethoxy)benzenesulfonyl chloride as an oil, which was suspended in anhydrous tetrahydrofuran (19.2 mL). To this solution was added ammonium hydroxide solution (28.0-30.0% NH₃ basis, 10.9 mL, 76.8 mmol, 20.0 eq.) and stirred at room temperature for 30 minutes. The reaction was quenched with aqueous hydrochloric acid (1.00 M, 20 mL) and extracted with ethyl acetate (3×20 mL). The combined organics were washed with brine (20 mL), dried (magnesium sulfate), filtered, and dry loaded onto Celite under reduced pressure. The crude was purified by flash column chromatography (80 g HP Silica, 0-60%, ethyl acetate/hexanes gradient) to afford the title compound as a white solid (580 mg). ¹H NMR (400 MHz, Chloroform-d) δ 7.95-7.89 (m, 1H), 7.51 (dd, 1H), 6.96-6.89 (m, 1H), 5.11 (s, 2H), 3.98 (d, 2H), 1.46-1.31 (m, 1H), 1.33-1.28 (m, 9H), 0.76-0.63 (m, 2H), 0.46-0.32 (m, 2H); LC-MS (method 4): Rₜ=1.46 min; MS (ESIpos): m/z=284 [M+H]⁺

### Intermediate 95

### 4-(tert-butyl)-2-iodo-1-(2,2,2-trifluoroethoxy)benzene

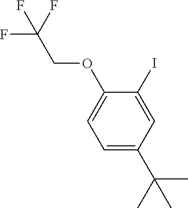 [SMILES: CC(C)(C)c1ccc(OCC(F)(F)F)c(I)c1]

To a stirred suspension of 4-tert-butyl-2-iodo-phenol (1.50 g, 5.43 mmol, 1.00 eq.) and potassium carbonate (3.00 g, 21.7 mmol, 4.00 eq.) in anhydrous N,N-dimethylformamide (2.72 mL, 2.00 M) was added 1,1,1-trifluoro-2-iodo-ethane (2.12 mL, 21.7 mmol, 4.00 eq.). The resulting brown suspension was heated at 130° C. for 24 h and then cooled to room temperature. The mixture was diluted with water (50 mL) and extracted with dichloromethane (3×50 mL). The combined organic extracts were washed with brine (50 mL), dried (magnesium sulfate), filtered and dry loaded onto Celite. The residue was purified by flash column chromatography (80 g HP Silica, 0-20% ethyl acetate/hexanes gradient) to give the title compound as a colourless oil (1.15 g). ¹H NMR (400 MHz, Chloroform-d) δ 7.79 (d, 1H), 7.32 (dd, 1H), 6.78 (d, 1H), 4.36 (q, 2H), 1.29 (s, 9H); LC-MS (method 5): Rₜ=3.49 min; MS (ESIpos): m/z=not detected.

### Intermediate 96

### 5-(tert-butyl)-2-(2,2,2-trifluoroethoxy)benzenesulfonamide

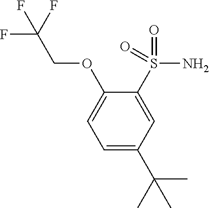 [SMILES: CC(C)(C)c1ccc(OCC(F)(F)F)c(S(N)(=O)=O)c1]

To a 40 mL vial under an argon atmosphere was added 4-tert-butyl-2-iodo-1-(2,2,2-trifluoroethoxy)benzene (1.15 g, 3.21 mmol, 1.00 eq), palladium(II) acetate (36.1 mg, 0.16 mmol, 5.00 mol %), CataCXium® A (92.1 mg, 0.26 mmol, 7.50 mol %), and 1,4-Diazabicyclo[2.2.2]octane bis(sulfur dioxide) adduct (463 mg, 1.93 mmol, 0.60 eq.) followed by the addition of degassed anhydrous isopropanol (16.1 mL, 0.20 M) and triethylamine (1.34 mL, 9.64 mmol, 3.00 eq.). The resulting red solution was heated at 75° C. for 16 h, cooled to room temperature and concentrated under reduced pressure. The residue was resuspended in anhydrous tetrahydrofuran (16.1 mL) and cooled to 0° C. in a ice/water bath. To this mixture was then added sulfuryl chloride (0.78 mL, 9.64 mmol, 3.00 eq.) dropwise, after which the resulting mixture was warmed to room temperature and stirred for a further 30 minutes. The reaction was quenched with ammonium chloride (30 mL), and the aqueous layer was extracted with dichloromethane (3×30 mL). The combined organics were washed with brine (30 mL), dried (magnesium sulfate), filtered, and dry loaded onto Celite under reduced pressure. The crude was purified by flash column chromatography (80 g HP Silica, 0-20%, ethyl acetate/hexanes gradient) to afford 5-tert-butyl-2-(2,2,2-trifluoroethoxy)benzenesulfonyl chloride as an oil, which was suspended in anhydrous tetrahydrofuran (16.1 mL). To this solution was added ammonium hydroxide solution (28.0-30.0% NH₃ basis, 9.14 mL, 64.3 mmol, 20.0 eq.) and stirred at room temperature for 30 minutes. The reaction was quenched with aqueous hydrochloric acid (1.00 M, 20 mL) and extracted with ethyl acetate (3×20 mL). The combined organics were washed with brine (20 mL), dried (magnesium sulfate), filtered, and dry loaded onto Celite under reduced pressure. The residue was purified by flash column chromatography (80 g HP Silica, 0-60%, ethyl acetate/hexanes gradient) to give the title compound as a white solid (324 mg). 1H NMR (400 MHz, Chloroform-d) δ 7.97 (d, 1H), 7.57 (dd, 1H), 7.02-6.95 (m, 1H), 4.98 (s, 2H), 4.53 (q, 2H), 1.32 (s, 9H); LC-MS (method 4): Rₜ=1.46 min; MS (ESIpos): m/z=312 [M+H]⁺

### Intermediate 97

### 4-(tert-butyl)-1-cyclobutoxy-2-iodobenzene

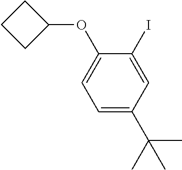 [SMILES: CC(C)(C)c1ccc(OC2CCC2)c(I)c1]

To a stirred suspension of 4-tert-butyl-2-iodo-phenol (1.50 g, 5.43 mmol, 1.00 eq.) and potassium carbonate (2.25 g, 16.3 mmol, 3.00 eq.) in anhydrous N,N-dimethylformamide (2.72 mL, 2.00 M) was added bromocyclobutane (1.53 mL, 16.3 mmol, 3.00 eq.). The resulting brown suspension was heated at 60° C. for 3 days and then cooled to room temperature. The mixture was diluted with water (50 mL) and extracted with dichloromethane (3×50 mL). The combined organic extracts were washed with brine (50 mL), dried (magnesium sulfate), filtered and dry loaded onto Celite. The residue was purified by flash column chromatography (80 g HP Silica, 0-20% ethyl acetate/hexanes gradient) to give the title compound as a colourless oil (1.44 g). ¹H NMR (400 MHz, Chloroform-d) δ 7.75 (d, 1H), 7.25 (dd, 1H), 6.60 (d, 1H), 4.71-4.58 (m, 1H), 2.52-2.38 (m, 2H), 2.32-2.16 (m, 2H), 1.94-1.80 (m, 1H), 1.76-1.59 (m, 1H), 1.27 (s, 9H); LC-MS (method 5): Rₜ=3.81 min; MS (ESIpos): m/z=not detected

### Intermediate 98

### 5-(tert-butyl)-2-cyclobutoxybenzenesulfonamide

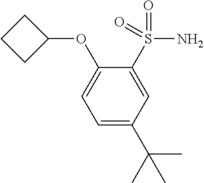 [SMILES: CC(C)(C)c1ccc(OC2CCC2)c(S(N)(=O)=O)c1]

To a 40 mL vial under an argon atmosphere was added 4-tert-butyl-1-(cyclobutoxy)-2-iodo-benzene (1.44 g, 4.37 mmol, 1.00 eq), palladium(II) acetate (49.0 mg, 0.22 mmol, 5.00 mol %), CataCXium® A (125 mg, 0.35 mmol, 7.50 mol %), and 1,4-Diazabicyclo[2.2.2]octane bis(sulfur dioxide) adduct (630 mg, 2.62 mmol, 0.60 eq.) followed by the addition of degassed anhydrous isopropanol (14.6 mL, 0.30 M) and triethylamine (1.82 mL, 13.1 mmol, 3.00 eq.). The resulting red solution was heated at 75° C. for 16 h, cooled to room temperature and concentrated under reduced pressure. The residue was resuspended in anhydrous tetrahydrofuran (14.6 mL) and cooled to 0° C. in a ice/water bath. To this mixture was then added sulfuryl chloride (1.06 mL, 13.1 mmol, 3.00 eq.) dropwise, after which the resulting mixture was warmed to room temperature and stirred for a further 30 minutes. The reaction was quenched with ammonium chloride (30 mL), and the aqueous layer was extracted with dichloromethane (3×30 mL). The combined organics were washed with brine (30 mL), dried (magnesium sulfate), filtered, and dry loaded onto Celite under reduced pressure. The residue was purified by flash column chromatography (80 g HP Silica, 0-20%, ethyl acetate/hexanes) to give 5-tert-butyl-2-(cyclobutoxy)benzenesulfonyl chloride as an oil, which was suspended in anhydrous tetrahydrofuran (14.6 mL). To this solution was added ammonium hydroxide solution (28.0-30.0% NH₃ basis, 12.4 mL, 87.3 mmol, 20.0 eq.) and stirred at room temperature for 30 minutes. The reaction was quenched with aqueous hydrochloric acid (1.00 M, 20 mL) and extracted with ethyl acetate (3×20 mL). The combined organic layers were washed with brine (20 mL), dried (magnesium sulfate), filtered, and dry loaded onto Celite under reduced pressure. The residue was purified by flash column chromatography (80 g HP Silica, 0-60%, ethyl acetate/hexanes) to give the title compound as a white solid (746 mg). ¹H NMR (400 MHz, Chloroform-d) δ 7.94-7.87 (m, 1H), 7.48 (dd, 1H), 6.86-6.77 (m, 1H), 5.03 (s, 2H), 4.87-4.74 (m, 1H), 2.57-2.44 (m, 2H), 2.33-2.18 (m, 2H), 1.99-1.84 (m, 1H), 1.81-1.65 (m, 1H), 1.33-1.28 (m, 9H); LC-MS (method 4): Rₜ=1.51 min; MS (ESIpos): m/z=284 [M+H]⁺

### Intermediate 99

### 4-(tert-butyl)-2-iodo-1-isopropoxybenzene

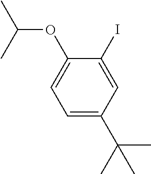

To a stirred suspension of 4-tert-butyl-2-iodo-phenol (1.50 g, 5.43 mmol, 1.00 eq.) and potassium carbonate (1.50 g, 10.9 mmol, 2.00 eq.) in anhydrous N,N-dimethylformamide (2.72 mL, 2.00 M) was added 2-bromopropane (1.02 mL, 10.9 mmol, 2.00 eq.). The resulting brown suspension was heated at 60° C. for 48 h and then cooled to room temperature. The mixture was diluted with water (50 mL) and extracted with dichloromethane (3×50 mL). The combined organic extracts were washed with brine (50 mL), dried (magnesium sulfate), filtered and dry loaded onto Celite. The residue was purified by flash column chromatography (80 g HP Silica, 0-20% ethyl acetate/hexanes gradient) to give the title compound as a colourless oil (1.63 g). ¹H NMR (400 MHz, Chloroform-d) δ 7.75 (d, 1H), 7.27 (dd, 1H), 6.75 (d, 1H), 4.51 (p, 1H), 1.38 (d, 6H), 1.28 (s, 9H); LC-MS (method 5): Rₜ=3.71 min; MS (ESIpos): m/z=not detected

### Intermediate 100

### 5-(tert-butyl)-2-isopropoxybenzenesulfonamide

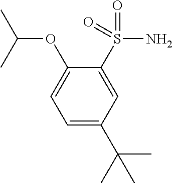 [SMILES: CC(C)Oc1ccc(C(C)(C)C)cc1S(N)(=O)=O]

To a 40 mL vial under an argon atmosphere was added 4-tert-butyl-2-iodo-1-isopropoxybenzene (1.63 g, 5.13 mmol, 1.00 eq), palladium(II) acetate (57.6 mg, 0.26 mmol, 5.00 mol %), CataCXium® A (147 mg, 0.41 mmol, 7.50 mol %), and 1,4-Diazabicyclo[2.2.2]octane bis(sulfur dioxide) adduct (740 mg, 3.08 mmol, 0.60 eq.) followed by the addition of degassed anhydrous isopropanol (17.1 mL, 0.30 M) and triethylamine (2.13 mL, 15.4 mmol, 3.00 eq.). The resulting red solution was heated at 75° C. for 16 h, cooled to room temperature and concentrated under reduced pressure. The residue was resuspended in anhydrous tetrahydrofuran (17.1 mL) and cooled to 0° C. in a ice/water bath. To this mixture was then added sulfuryl chloride (1.24 mL, 15.4 mmol, 3.00 eq.) dropwise, after which the resulting mixture was warmed to room temperature and stirred for a further 30 minutes. The reaction was quenched with ammonium chloride (30 mL), and extracted with dichloromethane (3×30 mL). The combined organic extracts were washed with brine (30 mL), dried (magnesium sulfate), filtered, and dry loaded onto Celite under reduced pressure. The residue was purified by flash column chromatography (80 g HP Silica, 0-20%, ethyl acetate/hexanes gradient) to give 5-tert-butyl-2-isopropoxy-benzenesulfonyl chloride as an oil, which was suspended in anhydrous tetrahydrofuran (17.1 mL). To this solution was added ammonium hydroxide solution (28.0-30.0% NH₃ basis, 14.6 mL, 103 mmol, 20.0 eq.) and stirred at room temperature for 30 minutes. The reaction was quenched with aqueous hydrochloric acid (1.00 M, 30 mL) and extracted with ethyl acetate (3×30 mL). The combined organic extracts were washed with brine (30 mL), dried (magnesium sulfate), filtered, and dry loaded onto Celite under reduced pressure. The residue was purified by flash column chromatography (80 g HP Silica, 0-60%, ethyl acetate/hexanes) to give the title compound as a white solid (720 mg). 1H NMR (400 MHz, Chloroform-d) δ 7.92 (d, 1H), 7.55-7.47 (m, 1H), 7.00-6.93 (m, 1H), 5.01 (s, 2H), 4.76 (hept, 1H), 1.44 (d, 6H), 1.33-1.28 (m, 9H); LC-MS (method 4): Rₜ=1.48 min; MS (ESIpos): m/z=272 [M+H]⁺

### Intermediate 101

### 1-Cyclobutoxy-2-iodo-4-isopropylbenzene

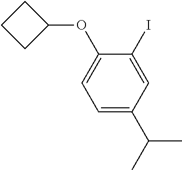

To a stirred solution of 2-iodo-4-isopropyl-phenol (2.00 g, 7.63 mmol, 1.00 eq.) in anhydrous N,N-dimethylformamide (15.3 mL, 0.50 M) was added potassium carbonate (2.11 g, 15.3 mmol, 2.00 eq.), and the reaction stirred for 5 minutes at room temperature. Bromocyclobutane (790 μL, 8.39 mmol, 1.10 eq.) was added, and the reaction was left stirring at 80° C. for 40 h. Additional bromocyclobutane (790 μL, 8.39 mmol, 1.10 eq.) and potassium carbonate (1.06 g, 7.65 mmol, 1.00 eq.) were added and the mixture was stirred at 80° C. for a further 24 h. The reaction was cooled to room temperature, diluted with water (30 mL) and extracted with dichloromethane (3×30 mL). The combined organic extracts were washed with brine (30 mL), dried (magnesium sulfate), filtered, and dry loaded onto Celite under reduced pressure. The residue was purified by flash column chromatography (80 g HP Silica, 0-10% ethyl acetate/hexanes gradient) to give the title compound as colorless oil (2.25 g). ¹H NMR (400 MHz, Chloroform-d) δ 7.65-7.59 (m, 1H), 7.13-7.05 (m, 1H), 6.63-6.56 (m, 1H), 4.64 (p, 1H), 2.88-2.73 (m, 1H), 2.51-2.39 (m, 2H), 2.32-2.17 (m, 2H), 1.94-1.80 (m, 1H), 1.76-1.59 (m, 1H), 1.24-1.17 (m, 6H); LC-MS (method 4): Rₜ=1.87 min; MS (ESIpos): m/z=not detected.

### Intermediate 102

### 2-Cyclobutoxy-5-isopropylbenzenesulfonyl chloride

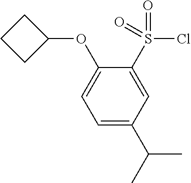 [SMILES: CC(C)c1ccc(OC2CCC2)c(S(=O)(=O)Cl)c1]

To an oven dried 40 mL vial under an argon atmosphere was added palladium(II) acetate (80.0 mg, 0.36 mmol, 5.00 mol %), CataCXium® A (204 mg, 0.57 mmol, 7.50 mol %), and 1,4-Diazabicyclo[2.2.2]octane bis(sulfur dioxide) adduct (1.03 g, 4.27 mmol, 0.60 eq.) followed by the addition of degassed anhydrous isopropanol (17.8 mL, 0.20 M), 1-(cyclobutoxy)-2-iodo-4-isopropyl-benzene (2.25 g, 7.12 mmol, 1.00 eq.), and triethylamine (2.96 mL, 21.4 mmol, 3.00 eq.). The resulting red solution was heated at 75° C. for 17 h, cooled to room temperature and concentrated under reduced pressure. The residue was resuspended in anhydrous tetrahydrofuran (17.8 mL) and cooled to 0° C. in a ice/water bath. To this mixture was then added sulfuryl chloride (1.73 mL, 21.4 mmol, 3.00 eq.) dropwise, after which the resulting mixture was warmed to room temperature and stirred for a further 30 minutes. The reaction was quenched with ammonium chloride (40 mL), and extracted with dichloromethane (3×40 mL). The combined organic extracts were washed with brine (40 mL), dried (magnesium sulfate), filtered, and dry loaded onto Celite under reduced pressure. The residue was purified by flash column chromatography (120 g Silica, 0-20%, ethyl acetate/hexanes gradient) to give the title compound as an oil (1.74 g). ¹H NMR (400 MHz, Chloroform-d) δ 7.80-7.75 (m, 1H), 7.50-7.42 (m, 1H), 6.90-6.83 (m, 1H), 4.83 (p, 1H), 2.98-2.83 (m, 1H), 2.57-2.46 (m, 1H), 2.41-2.26 (m, 2H), 2.02-1.64 (m, 3H), 1.28-1.19 (m, 6H); LC-MS (method 4): Rₜ=1.76 min; MS (ESIneg): m/z=not detected

### Intermediate 103

### 2-cyclobutoxy-5-isopropylbenzenesulfonamide

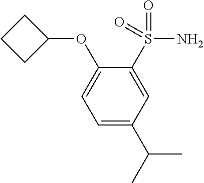 [SMILES: CC(C)c1ccc(OC2CCC2)c(S(N)(=O)=O)c1]

To a stirred solution of 2-(cyclobutoxy)-5-isopropyl-benzenesulfonyl chloride (1.74 g, 6.01 mmol, 1.00 eq.) in anhydrous tetrahydrofuran (10.0 mL, 0.60 M) was added ammonium hydroxide solution (28.0-30.0% NH₃ basis, 16.7 mL, 120 mmol, 20.0 eq.) and stirred at room temperature for 30 minutes. The reaction was quenched with hydrochloric acid (1.00 M, 20 mL), and the aqueous layer was extracted with ethyl acetate (3×20 mL). The combined organic extracts were washed with brine (20 mL), dried (magnesium sulfate), filtered, and dry loaded onto Celite under reduced pressure. The crude was purified by flash column chromatography (80 g HP Silica, 0-60%, ethyl acetate/hexanes gradient) to afford the title compound as a white solid (536 mg). 1H NMR (400 MHz, Chloroform-d) 6 7.80-7.74 (m, 1H), 7.37-7.28 (m, 1H), 6.85-6.77 (m, 1H), 5.02 (s, 2H), 4.80 (p, 1H), 2.98-2.83 (m, 1H), 2.57-2.43 (m, 2H), 2.34-2.19 (m, 2H), 1.99-1.85 (m, 1H), 1.81-1.64 (m, 1H), 1.27-1.20 (m, 6H); LC-MS (method 4): Rₜ=1.47 min; MS (ESIpos): m/z=270 [M+H]⁺

### Intermediate 104

### 5-(tert-Butyl)-2-methoxybenzenesulfonamide

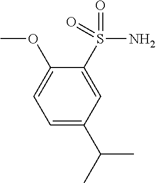

To a stirred solution of 5-tert-butyl-2-methoxy-benzenesulfonyl chloride (1.00 g, 3.81 mmol, 1.00 eq.) in anhydrous tetrahydrofuran (6.34 mL, 0.60 M) was added ammonium hydroxide solution (28.0-30.0% NH₃ basis, 10.6 mL, 76.1 mmol, 20.0 eq.) and stirred at room temperature for 30 minutes. The reaction was quenched with hydrochloric acid (1.00 M, 20 mL), and extracted with ethyl acetate (3×20 mL). The combined organic extracts were washed with brine (20 mL), dried (magnesium sulfate), filtered, and dry loaded onto Celite under reduced pressure. The residue was purified by flash column chromatography (80 g HP Silica, 0-60%, ethyl acetate/hexanes gradient) to give the title compound as a white solid (678 mg). ¹H NMR (400 MHz, Chloroform-d) δ 7.96-7.90 (m, 1H), 7.59-7.51 (m, 1H), 6.98 (d, 1H), 5.01 (s, 2H), 4.02-3.97 (m, 3H), 1.34-1.30 (m, 9H); LC-MS (method 4): Rₜ=1.37 min; MS (ESIpos): m/z=244 [M+H]⁺

### Intermediate 105

### 1-[(2,2-Difluorocyclopropyl)methoxy]-2-iodo-4-isopropylbenzene

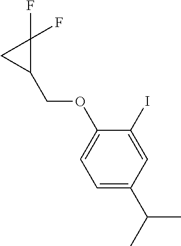 [SMILES: CC(C)c1ccc(OCC2CC2(F)F)c(I)c1]

To a stirred solution of 2-iodo-4-isopropyl-phenol (1.28 g, 4.87 mmol, 1.00 eq) in anhydrous N,N-dimethylformamide (9.74 mL, 0.50 M) was added potassium carbonate (1.35 g, 9.74 mmol, 2.00 eq), and the reaction stirred for 5 minutes at room temperature. 2-(Bromomethyl)-1,1-difluoro-cyclopropane (1.00 g, 5.84 mmol, 1.20 eq) was added, and the reaction was heated at 70° C. for 20 h. The reaction was cooled to room temperature, diluted with water (20 mL) and extracted with dichloromethane (3×20 mL). The combined organic extracts were washed with brine (20 mL), dried (magnesium sulfate), filtered and dry loaded onto Celite under reduced pressure. The residue was purified by flash column chromatography (80 g HP silica, 0-20% ethyl acetate/hexanes gradient) to give the title compound as a clear oil (938 mg). 1H NMR (400 MHz, Chloroform-d) δ 7.67-7.61 (m, 1H), 7.18-7.10 (m, 1H), 6.78-6.71 (m, 1H), 4.22-4.12 (m, 1H), 4.04-3.94 (m, 1H), 2.82 (hept, 1H), 2.20-2.03 (m, 1H), 1.67-1.54 (m, 1H), 1.42-1.29 (m, 1H), 1.25-1.18 (m, 6H); LC-MS (method 4): Rₜ=1.77 min; MS (ESIpos): m/z=not detected

### Intermediate 106

### 2-[(2,2-Difluorocyclopropyl)methoxy]-5-isopropylbenzenesulfonyl chloride

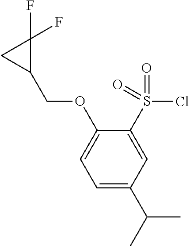 [SMILES: CC(C)c1ccc(OCC2CC2(F)F)c(S(=O)(=O)Cl)c1]

To an oven dried 40 mL vial under an argon atmosphere was added palladium(II) acetate (29.9 mg, 0.13 mmol, 5.00 mol %), CataCXium® A (76.4 mg, 0.21 mmol, 7.50 mol %), and 1,4-Diazabicyclo[2.2.2]octane bis(sulfur dioxide) adduct (384 mg, 1.60 mmol, 0.60 eq.) followed by the addition of degassed anhydrous isopropanol (13.3 mL, 0.20 M), 1-[(2,2-difluorocyclopropyl)methoxy]-2-iodo-4-isopropyl-benzene (938 mg, 2.66 mmol, 1.00 eq.), and triethylamine (1.11 mL, 7.99 mmol, 3.00 eq.). The resulting red solution was heated at 75° C. for 88 h, cooled to room temperature and concentrated under reduced pressure. The residue was resuspended in anhydrous tetrahydrofuran (13.3 mL) and cooled to 0° C. in a ice/water bath. To this mixture was then added sulfuryl chloride (650 μL, 7.99 mmol, 3.00 eq.) dropwise, after which the resulting mixture was warmed to room temperature and stirred for a further 30 minutes. The reaction was quenched with ammonium chloride (30 mL) and extracted with dichloromethane (3×30 mL). The combined organics were washed with brine (30 mL), dried (magnesium sulfate), filtered, and dry loaded onto Celite under reduced pressure. The residue was purified by flash column chromatography (80 g HP Silica, 0-20%, ethyl acetate/hexanes gradient) to give the title compound as an oil (227 mg). ¹H NMR (400 MHz, Chloroform-d) δ 7.81 (d, 1H), 7.55-7.49 (m, 1H), 7.05-6.98 (m, 1H), 4.43-4.31 (m, 1H), 4.22-4.12 (m, 1H), 3.04-2.86 (m, 1H), 2.29-2.10 (m, 1H), 1.75-1.60 (m, 1H), 1.51-1.35 (m, 1H), 1.28-1.24 (m, 6H); LC-MS (method 4): Rₜ=1.68 min; MS (ESIpos): m/z=not detected

### Intermediate 107

### 2-((2,2-difluorocyclopropyl)methoxy)-5-isopropylbenzenesulfonamide

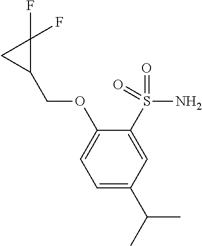 [SMILES: CC(C)c1ccc(OCC2CC2(F)F)c(S(N)(=O)=O)c1]

To a stirred solution of 2-[(2,2-difluorocyclopropyl)methoxy]-5-isopropyl-benzenesulfonyl chloride (227 mg, 0.70 mmol, 1.00 eq) in anhydrous tetrahydrofuran (1.16 mL, 0.60 mmol) was added ammonium hydroxide solution (20% in water, 2.72 mL, 14.0 mmol, 20.0 eq) and stirred at room temperature for 30 minutes. The reaction was quenched with hydrochloric acid (1.00 M, 10 mL), and extracted with ethyl acetate (3×10 mL). The combined organics were washed with brine (10 mL), dried (magnesium sulfate), filtered, and dry loaded onto Celite under reduced pressure. The residue was purified by flash column chromatography (24 g HP Silica, 0-60%, ethyl acetate/hexanes gradient) to give the title compound as a white solid (156 mg). 1H NMR (400 MHz, Chloroform-d) δ 7.82-7.76 (m, 1H), 7.42-7.34 (m, 1H), 6.95 (d, 1H), 4.99 (s, 2H), 4.35-4.25 (m, 1H), 4.27-4.17 (m, 1H), 3.00-2.85 (m, 1H), 2.25-2.09 (m, 1H), 1.72-1.58 (m, 1H), 1.52-1.39 (m, 1H), 1.30-1.21 (m, 6H); LC-MS (method 4): Rₜ=1.45 min; MS (ESIpos): m/z=306 [M+H]⁺

### Intermediate 108

### 4,5-Difluoro-2-iodophenol

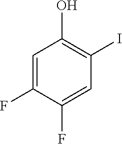

To a 0° C. solution of iodine (4.88 g, 19.2 mmol, 1.00 eq.), potassium iodide (3.19 g, 19.2 mmol, 1.00 eq.), and 3,4-difluorophenol (2.50 g, 19.2 mmol, 1.00 eq.) in water (64.1 mL, 0.30 M) was added sodium hydroxide solution (2.00 M, 19.22 mL, 38.4 mmol, 2.00 eq.). The resulting mixture was warmed to room temperature and stirred for 16 h. The reaction mixture was diluted with saturated ammonium chloride (50 mL) followed by the addition of aqueous sodium thiosulfate (50% w/w) and stirred at room temperature for 20 minutes. The mixture was extracted with diethyl ether (3×40 mL), and the combined organic extracts were washed with brine (40 mL), dried (magnesium sulfate), filtered, and dry loaded onto Celite under reduced pressure. The residue was purified by flash column chromatography (120 g Silica, 0-20%, ethyl acetate/hexanes gradient) to give the title compound as a colorless oil (2.72 g). 1H NMR (400 MHz, Chloroform-d) δ 7.52-7.40 (m, 1H), 6.91-6.80 (m, 1H), 5.17 (s, 1H); LC-MS (method 4): Rₜ=1.39 min; MS (ESIneg): m/z=254 [M−H]⁻

### Intermediate 109 1-Ethoxy-4,5-difluoro-2-iodobenzene

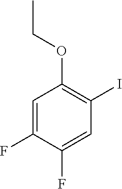

To a room temperature stirred solution of 4,5-difluoro-2-iodophenol (2.72 g, 10.63 mmol, 1.00 eq.) in anhydrous N,N-dimethylformamide (35.4 mL, 0.20 M) was added potassium carbonate (2.94 g, 21.3 mmol, 2.00 eq.) and iodoethane (1.28 mL, 15.9 mmol, 1.50 eq.). The resulting mixture was heated at 60° C. for 64 h and then cooled to room temperature. The mixture was diluted with water (30 mL) and extracted with ethyl acetate (3×30 mL). The combined organic extracts were washed with brine (30 mL), dried (magnesium sulfate), filtered, and dry loaded onto Celite under reduced pressure. The residue was purified by flash column chromatrography (120 g Silica, 0-20%, ethyl acetate/hexanes gradient) to give the title compound as an oil (1.40 g). ¹H NMR (400 MHz, Chloroform-d) δ 7.62-7.53 (m, 1H), 6.70-6.60 (m, 1H), 4.02 (q, 2H), 1.47 (t, 3H); LC-MS (method 4): Rₜ=1.62 min; MS (ESIpos): m/z=not detected

### Intermediate 110

### 2-Ethoxy-4,5-difluorobenzenesulfonamide

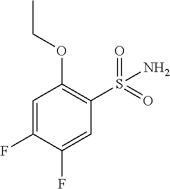

To a 40 mL vial was added 1-ethoxy-4,5-difluoro-2-iodo-benzene (1.40 g, 4.94 mmol, 1.00 eq), palladium(II) acetate (55.4 mg, 0.25 mmol, 5.00 mol %), CataCXium® A (142 mg, 0.39 mmol, 7.50 mol %), and 1,4-Diazabicyclo[2.2.2]octane bis(sulfur dioxide) adduct (712 mg, 2.96 mmol, 0.60 eq.) sequentially followed by the addition of degassed anhydrous isopropanol (24.7 mL, 0.20 M) and triethylamine (2.05 mL, 14.8 mmol, 3.00 eq.). The resulting red solution was heated at 75° C. for 16 h, cooled to room temperature and concentrated under reduced pressure. The residue was resuspended in anhydrous tetrahydrofuran (25.0 mL) and cooled to 0° C. in a ice/water bath. To this mixture was then added sulfuryl chloride (1.20 mL, 14.8 mmol, 3.00 eq.) dropwise, after which the resulting mixture was warmed to room temperature and stirred for a further 30 minutes. The reaction was quenched with ammonium chloride (30 mL), and the aqueous layer was extracted with ethyl acetate (3×30 mL). The combined organics were washed with brine (30 mL), dried (magnesium sulfate), filtered, and concentrated down under reduced pressure. The residue was dissolved in tetrahydrofuran (25.0 mL) and to this solution was added ammonium hydroxide solution (28.0-30.0% NH₃ basis, 14.0 mL, 98.7 mmol, 20.0 eq.) and the resulting mixture was stirred at room temperature for 30 minutes. The reaction was quenched with aqueous hydrochloric acid (1.00 M, 30 mL) and extracted with ethyl acetate (3×30 mL). The combined organics were washed with brine (30 mL), dried (magnesium sulfate), filtered, and dry loaded onto Celite under reduced pressure. The residue was purified by flash column chromatography (40 g HP Silica, 0-60%, ethyl acetate/hexanes gradient) to give the title compound as a white solid (262 mg). ¹H NMR (400 MHz, Chloroform-d) δ 7.83-7.74 (m, 1H), 6.91-6.82 (m, 1H), 5.00 (s, 2H), 4.21 (q, 2H), 1.57-1.49 (m, 3H), LC-MS (method 4): Rₜ=1.22 min; MS (ESIpos): m/z=238 [M+H]⁺

### Intermediate 111

### 4-(Hydroxymethyl)-2-iodophenol

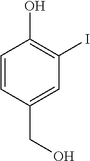

To a stirred suspension of 4-(hydroxymethyl)phenol (1.24 g, 10.0 mmol, 1.00 eq.) in acetonitrile (20.0 mL, 0.50 M) was added N-iodosuccinimide (2.25 g, 10.0 mmol, 1.00 eq.). The resulting mixture was stirred at room temperature for 30 minutes. The reaction was quenched with aqueous sodium thiosulfate (10% w/w, 20 mL) and extracted with ethyl acetate (3×20 mL). The combined organic extracts were washed with brine (20 mL), dried (magnesium sulfate), filtered, and dry loaded onto Celite under reduced pressure. The residue was purified by flash column chromatography (80 g HP Silica, 0-100%, ethyl acetate/hexanes gradient) to give the title compound as a solid (197 mg). ¹H NMR (400 MHz, DMSO-d₆) δ 10.19 (s, 1H), 7.63-7.57 (m, 1H), 7.15-7.07 (m, 1H), 6.86-6.79 (m, 1H), 5.08-5.00 (m, 1H), 4.37-4.31 (m, 2H); LC-MS (method 4): Rₜ=1.10 min; MS (ESIneg): m/z=249 [M−H]⁻

### Intermediate 112

### (3-Iodo-4-methoxyphenyl)methanol

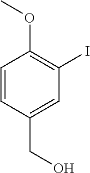

To a stirred solution of 4-(hydroxymethyl)-2-iodo-phenol (190 mg, 0.76 mmol, 1.00 eq.) in tetrahydrofuran (3.80 mL, 0.20 M) was added potassium carbonate (420 mg, 3.04 mmol, 4.00 eq.) and iodomethane (140 μL, 2.28 mmol, 3.00 eq.). The resulting mixture was heated at 50° C. for 16 h and then cooled to room temperature. The reaction mixture was diluted with water (10 mL) and extracted with ethyl acetate (3×10 mL). The combined organic extracts were washed with brine (10 mL), dried (magnesium sulfate), filtered, and dry loaded onto Celite under reduced pressure. The residue was purified by flash column chromatography (12 g HP Silica, 0-100, ethyl acetate/hexanes gradient) to give the title compound as a yellow solid (147 mg). 1H NMR (400 MHz, DMSO-d₆) δ 7.73-7.67 (m, 1H), 7.32-7.24 (m, 1H), 6.99-6.92 (m, 1H), 5.15 (t, 1H), 4.42-4.36 (m, 2H), 3.80 (s, 3H), LC-MS (method 4): Rₜ=1.27 min; MS (ESIneg): m/z=not detected

### Intermediate 113

### 2-Iodo-1-methoxy-4-(methoxymethyl)benzene

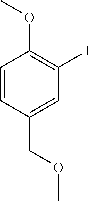

To a stirred suspension of (3-iodo-4-methoxy-phenyl)methanol (142 mg, 0.54 mmol, 1.00 eq.) in anhydrous tetrahydrofuran (2.69 mL, 0.20 M) was added sodium hydride (60% dispersion in mineral oil, 43.0 mg, 1.08 mmol, 2.00 eq.) and iodomethane (50.0 μL, 0.81 mmol, 1.50 eq.). The resulting mixture was stirred at room temperature for 16 h. The reaction mixture was diluted with water (10 mL) and extracted with ethyl acetate (3×10 mL). The combined organics were washed with brine (10 mL), dried (magnesium sulfate), filtered, and dry loaded onto Celite under reduced pressure. The residue was purified by flash column chromatography (12 g HP Silica, 0-100, ethyl acetate/hexanes gradient) to give the title compound as a colorless oil (110 mg). ¹H NMR (400 MHz, Chloroform-d) δ 7.79-7.73 (m, 1H), 7.31-7.23 (m, 1H), 6.83-6.76 (m, 1H), 4.35 (s, 2H), 3.88 (s, 3H), 3.36 (s, 3H) LC-MS (method 4): Rₜ=1.47 min; MS (ESIpos): m/z=not detected

### Intermediate 114

### 2-Methoxy-5-(methoxymethyl)benzenesulfonamide

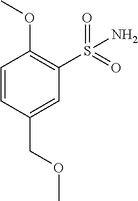

To a 20 mL vial was added 2-iodo-1-methoxy-4-(methoxymethyl)benzene (110 mg, 0.40 mmol, 1.00 eq.), palladium(II) acetate (4.44 mg, 0.02 mmol, 5.00 mol %), CataCXium® A (11.4 mg, 0.03 mmol, 7.50 mol %), and 1,4-Diazabicyclo[2.2.2]octane bis(sulfur dioxide) adduct (57.0 mg, 0.24 mmol, 0.60 eq.) sequentially followed by the addition of degassed anhydrous isopropanol (1.98 mL, 0.20 M) and triethylamine (160 μL, 1.19 mmol, 3.00 eq.). The resulting red solution was heated at 75° C. for 16 h, cooled to room temperature and concentrated under reduced pressure. The residue was resuspended in anhydrous tetrahydrofuran (2.00 mL) and cooled to 0° C. in a ice/water bath. To this mixture was then added sulfuryl chloride (100 μL, 1.19 mmol, 3.00 eq.) dropwise, after which the resulting mixture was warmed to room temperature and stirred for a further 30 minutes. The reaction was quenched with ammonium chloride (10 mL), and the aqueous layer was extracted with ethyl acetate (3×10 mL). The combined organic extracts were washed with brine (10 mL), dried (magnesium sulfate), filtered, and concentrated under reduced pressure. The residue was suspended in anhydrous tetrahydrofuran (2.00 mL). To this solution was then added ammonium hydroxide solution (28.0-30.0% NH₃ basis, 1.13 mL, 7.91 mmol, 20.0 eq.) and the resulting mixture was stirred at room temperature for 30 minutes. The reaction was diluted with aqueous hydrochloric acid (1.00 M in water, 10 mL) and extracted with ethyl acetate (3×10 mL). The combined organic extracts were washed with brine (10 mL), dried (magnesium sulfate), filtered, and dry loaded onto Celite under reduced pressure. The residue was purified by flash column chromatography (12 g HP Silica, 0-80%, ethyl acetate/hexanes) to afford the title compound as a white solid (12 mg). 1H NMR (400 MHz, Chloroform-d) δ 7.92-7.87 (m, 1H), 7.58-7.50 (m, 1H), 7.04 (d, 1H), 5.00 (s, 2H), 4.42 (s, 2H), 4.02 (s, 3H), 3.38 (s, 3H); LC-MS (method 4): Rₜ=1.06 min; MS (ESIpos): m/z=232 [M+H]⁺

### Intermediate 115

### 1-Cyclopropyl-4-(methoxymethoxy)benzene

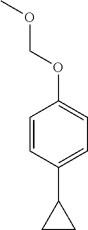

To a 0° C. stirred solution of 4-cyclopropylphenol (4.95 g, 36.9 mmol, 1.00 eq.) in anhydrous dichloromethane (123 mL, 0.30 M) was added chloromethyl methyl ether (4.20 mL, 55.3 mmol, 1.50 eq.) dropwise followed by N-ethyldiisopropylamine (12.6 mmol, 73.8 mmol, 2.00 eq). The resulting solution was warmed to room temperature and stirred for 40 h, after which additional chloromethyl methyl ether (2.8 mL, 36.9 mmol, 1.00 eq) and N-ethyldiisopropylamine (6.30 mL, 36.9 mmol, 1.00 eq) were added and stirred at room temperature. After 24 h, the mixture was diluted with water (200 mL) and extracted with dichloromethane (3×100 mL). The combined organic extracts were washed with hydrochloric acid (1.00 M in water, 200 mL), aqueous sodium hydroxide (1.00 M, 200 mL), brine (200 mL), dried (magnesium sulfate), filtered, and dry loaded onto Celite under reduced pressure. The residue was purified by flash column chromatography (330 g Silica, 0-20% ethyl acetate/hexanes gradient) to give the title compound as a colourless oil (6.76 g). ¹H NMR (400 MHz, Chloroform-d) δ 7.01 (d, 2H), 6.97-6.91 (m, 2H), 5.17-5.12 (m, 2H), 3.51-3.45 (m, 3H), 1.92-1.80 (m, 1H), 0.95-0.84 (m, 2H), 0.70-0.56 (m, 2H), LC-MS (method 4): Rₜ=1.55 min; MS (ESIpos): m/z=not detected

### Intermediate 116

### 4-Cyclopropyl-2-iodo-1-(methoxymethoxy)benzene

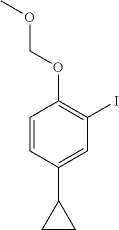

To a stirred solution of 1-cyclopropyl-4-(methoxymethoxy)benzene (5.67 g, 31.8 mmol, 1.00 eq.) in anhydrous tetrahydrofuran (106 mL, 0.30 M) was added n-butyllithium solution (2.50 M in hexanes, 14.6 mL, 0.75 mmol, 1.15 eq.) dropwise, afterwhich the solution was warmed to room temperature and stirred for 30 minutes. The mixture was then cooled back to −78° C. and a solution of iodine (9.37 g, 36.9 mmol, 1.16 eq.) in anhydrous tetrahydrofuran (110 mL) was added dropwise via cannula. After complete addition, the mixture was warmed to room temperature and stirred for 30 minutes. The mixture was then quenched with sodium thiosulfate (10 wt. % in water, 100 mL), partially concentrated under reduced pressure and extracted with diethyl ether (3×100 mL). The combined organic extracts were washed with brine (100 mL), dried (magnesium sulfate), filtered, and concentrated under reduced pressure. The residue was purified by flash reverse phase column chromatography (330 g Silica, 40-100% acetonitrile/water buffered with 0.1% formic acid gradient) to give the title compound as a brown oil (1.96 g). ¹H NMR (400 MHz, Chloroform-d) δ 7.53-7.48 (m, 1H), 7.03-6.91 (m, 2H), 5.19 (s, 2H), 3.54-3.48 (m, 3H), 1.87-1.75 (m, 1H), 0.98-0.85 (m, 2H), 0.69-0.55 (m, 2H), LC-MS (method 4): Rₜ=1.63 min; MS (ESIpos): m/z=not detected

### Intermediate 117

### 4-Cyclopropyl-2-iodophenol

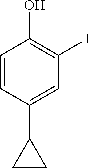 [SMILES: Oc1ccc(C2CC2)cc1I]

To a 0° C. stirred solution of sodium iodide (1.93 g, 12.9 mmol, 2.00 eq.) and 4-cyclopropyl-2-iodo-1-(methoxymethoxy)benzene (1.96 g, 6.43 mmol, 1.00 eq.) in anhydrous acetonitrile (32.1 mL, 0.20 M) was added trimethylsilyl chloride (1.22 mL, 9.64 mmol, 1.50 eq.) dropwise. After stirring at 0° C. for a further 10 minutes, the mixture was warmed to room temperature and stirred for 16 h. The reaction mixture was diluted with aqueous sodium thiosulfate (10% w/w, 30 mL) and extracted with diethyl ether (3×30 mL). The combined organic extracts were dried (sodium sulfate), filtered and dry loaded onto Celite under reduced pressure. The residue was purified using flash column chromatography (80 g HP Silica, 0-100%, ethyl acetate/hexanes gradient) to give the title compound as an off-white solid (1.46 g). ¹H NMR (400 MHz, Chloroform-d) δ 7.41-7.35 (m, 1H), 6.96 (dd, 1H), 6.90-6.85 (m, 1H), 5.07 (s, 1H), 1.86-1.75 (m, 1H), 0.97-0.84 (m, 2H), 0.67-0.53 (m, 2H), LC-MS (method 4): Rₜ=1.46 min; MS (ESIneg): m/z=259 [M−H]⁻

### Intermediate 118

### 4-Cyclopropyl-2-iodo-1-methoxybenzene

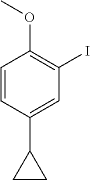

To a stirred suspension of 4-cyclopropyl-2-iodo-phenol (730 mg, 2.81 mmol, 1.00 eq.) in anhydrous N,N-dimethylformamide (14.0 mL, 0.20 M) was added potassium carbonate (776 mg, 5.61 mmol, 2.00 eq.) and iodomethane (260 μL, 4.21 mmol, 1.20 eq.). The resulting mixture was heated at 70° C. for 16 h and then cooled to room temperature. The reaction mixture was diluted with water (20 mL) and extracted with ethyl acetate (3×20 mL). The combined organic extracts were washed with brine (20 mL), dried (magnesium sulfate), filtered, and dry loaded onto Celite under reduced pressure. The residue was purified by flash column chromatography (40 g HP Silica, 0-30%, ethyl acetate/hexanes gradient) to give the title compound as a colorless oil (365 mg). ¹H NMR (400 MHz, Chloroform-d) δ 7.52-7.47 (m, 1H), 7.06-6.99 (m, 1H), 6.75-6.68 (m, 1H), 3.84 (s, 3H), 1.87-1.75 (m, 1H), 0.97-0.84 (m, 2H), 0.68-0.54 (m, 2H), LC-MS (method 4): Rₜ=1.64 min; MS (ESIpos): m/z=not detected

### Intermediate 119

### 5-Cyclopropyl-2-methoxybenzenesulfonamide

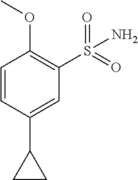

To a 20 mL vial was added 4-cyclopropyl-2-iodo-1-methoxy-benzene (359 mg, 1.31 mmol, 1.00 eq.), palladium(II) acetate (14.7 mg, 0.07 mmol, 5.00 mol %), CataCXium® A (37.6 mg, 0.10 mmol, 7.50 mol %), and 1,4-Diazabicyclo[2.2.2]octane bis(sulfur dioxide) adduct (189 mg, 0.79 mmol, 0.60 eq.) sequentially followed by the addition of degassed anhydrous isopropanol (6.55 mL, 0.20 M) and triethylamine (540 μL, 3.93 mmol, 3.00 eq.). The resulting red solution was heated at 75° C. for 16 h, cooled to room temperature and concentrated under reduced pressure. The residue was resuspended in anhydrous tetrahydrofuran (7.00 mL) and cooled to 0° C. in a ice/water bath. To this mixture was then added sulfuryl chloride (320 μL, 3.93 mmol, 3.00 eq.) dropwise, after which the resulting mixture was warmed to room temperature and stirred for a further 30 minutes. The reaction was quenched with ammonium chloride (20 mL), and the aqueous layer was extracted with ethyl acetate (3×20 mL). The combined organic extracts were washed with brine (20 mL), dried (magnesium sulfate), filtered and concentrated under reduced pressure. The residue was suspended in anhydrous tetrahydrofuran (7.00 mL). To this solution was then added ammonium hydroxide solution (28.0-30.0% NH₃ basis, 3.73 mL, 26.2 mmol, 20.0 eq.) and the resulting mixture stirred at room temperature for 30 minutes. The reaction was quenched with aqueous hydrochloric acid (1.00 M, 20 mL) and extracted with ethyl acetate (3×20 mL). The combined organic extracts were washed with brine (20 mL), dried (magnesium sulfate), filtered and dry loaded onto Celite under reduced pressure. The residue was purified by flash column chromatography (24 g HP Silica, 0-80%, ethyl acetate/hexanes gradient) to give the title compound as a white solid (96.0 mg). ¹H NMR (400 MHz, Chloroform-d) δ 7.64-7.59 (m, 1H), 7.28 (d, 1H), 6.98-6.91 (m, 1H), 4.99 (s, 2H), 3.98 (s, 3H), 1.95-1.84 (m, 1H), 1.03-0.89 (m, 2H), 0.73-0.59 (m, 2H), LC-MS (method 4): Rₜ=1.23 min; MS (ESIpos): m/z=228 [M+H]⁺

### Intermediate 120

### 4-Cyclopropyl-1-ethoxy-2-iodobenzene

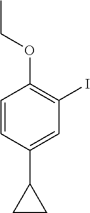

To a stirred solution of 4-cyclopropyl-2-iodo-phenol (730 mg, 2.81 mmol, 1.00 eq.) in anhydrous N,N-dimethylformamide (14.0 mL, 0.20 M) was added potassium carbonate (776 mg, 5.61 mmol, 2.00 eq.) and iodoethane (270 μL, 3.37 mmol, 1.20 eq.). The resulting mixture was heated at 70° C. for 16 h and then cooled to room temperature. The reaction mixture was diluted with water (20 mL) and extracted with ethyl acetate (3×20 mL). The combined organic extracts were washed with brine (20 mL), dried (magnesium sulfate), filtered and dry loaded onto Celite under reduced pressure. The residue was purified by flash column chromatography (40 g HP Silica, 0-30%, ethyl acetate/hexanes gradient) to give the title compound as a colorless oil (760 mg). 1H NMR (400 MHz, Chloroform-d) δ 7.52-7.46 (m, 1H), 7.03-6.96 (m, 1H), 6.73-6.66 (m, 1H), 4.05 (q, 2H), 1.86-1.74 (m, 1H), 1.45 (t, 3H), 0.96-0.83 (m, 2H), 0.67-0.54 (m, 2H); LC-MS (method 4): Rₜ=1.71 min; MS (ESIpos): m/z=not detected

### Intermediate 121

### 5-Cyclopropyl-2-ethoxybenzenesulfonamide

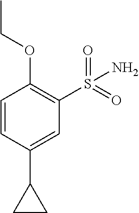 [SMILES: CCOc1ccc(C2CC2)cc1S(N)(=O)=O]

To a 20 mL vial was added 4-cyclopropyl-1-ethoxy-2-iodo-benzene (756 mg, 2.62 mmol, 1.00 eq.), palladium(II) acetate (29.5 mg, 0.13 mmol, 5.00 mol %), CataCXium® A (75.3 mg, 0.21 mmol, 7.50 mol %), and 1,4-Diazabicyclo[2.2.2]octane bis(sulfur dioxide) adduct (378 mg, 1.57 mmol, 0.60 eq.) sequentially followed by the addition of degassed anhydrous isopropanol (13.1 mL, 0.20 M) and triethylamine (1.09 mL, 7.87 mmol, 3.00 eq.). The resulting red solution was heated at 75° C. for 16 h, cooled to room temperature and concentrated under reduced pressure. The residue was resuspended in anhydrous tetrahydrofuran (13.0 mL) and cooled to 0° C. in a ice/water bath. To this mixture was then added sulfuryl chloride (640 μL, 7.87 mmol, 3.00 eq.) dropwise, after which the resulting mixture was warmed to room temperature and stirred for a further 30 minutes. The reaction mixture was diluted with ammonium chloride (20 mL) and extracted with ethyl acetate (3×20 mL). The combined organic extracts were washed with brine (20 mL), dried (magnesium sulfate), filtered and concentrated down under reduced pressure. The residue was resuspended in anhydrous tetrahydrofuran (13.0 mL). To this solution was then added ammonium hydroxide solution (28.0-30.0% NH₃ basis, 7.46 mL, 52.5 mmol, 20.0 eq.) and the resulting mixture was stirred at room temperature for 30 minutes. The reaction was quenched with aqueous hydrochloric acid (1.00 M, 30 mL) and extracted with ethyl acetate (3×20 mL). The combined organic extracts were washed with brine (20 mL), dried (magnesium sulfate), filtered and dry loaded onto Celite under reduced pressure. The reside was purified by flash column chromatography (40 g HP Silica, 0-80%, ethyl acetate/hexanes gradient) to give the title compound as a white solid (159 mg). ¹H NMR (400 MHz, Chloroform-d) δ 7.64-7.58 (m, 1H), 7.28-7.20 (m, 1H), 6.96-6.89 (m, 1H), 4.99 (s, 2H), 4.21 (q, 2H), 1.95-1.83 (m, 1H), 1.50 (t, 3H), 1.00-0.89 (m, 2H), 0.69-0.61 (m, 2H), LC-MS (method 4): Rₜ=1.30 min; MS (ESIpos): m/z=242 [M+H]+

### Intermediate 122

### tert-Butyl 4-[(4-(tert-butyl)-2-iodophenoxy)methyl]piperidine-1-carboxylate

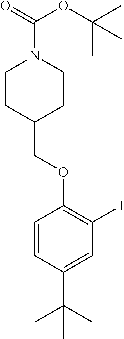 [SMILES: CC(C)(C)OC(=O)N1CCC(COc2ccc(C(C)(C)C)cc2I)CC1]

To a stirred solution of 4-tert-butyl-2-iodo-phenol (1.50 g, 5.43 mmol, 1.00 eq.) in anhydrous N,N-dimethylformamide (18.1 mL, 0.30 M) was added potassium carbonate (1.50 g, 10.9 mmol, 2.00 eq.) and tert-butyl 4-(bromomethyl)piperidine-1-carboxylate (2.27 g, 8.15 mmol, 1.50 eq.). The resulting mixture was heated at 75° C. for 64 h and then cooled to room temperature. The reaction mixture was diluted with water (30 mL) and extracted with dichloromethane (3×30 mL). The combined organic extracts were 10 washed with brine (30 mL), dried (magnesium sulfate), filtered and dry loaded onto Celite under reduce pressure. The reside was purified by flash column chromatography (80 g HP Silica, 0-20%, ethyl acetate/hexanes gradient) to give the title compound as a white solid (2.35 g). 1H NMR (400 MHz, Chloroform-d) δ 7.76 (d, 1H), 7.32-7.24 (m, 1H), 6.74-6.67 (m, 1H), 4.18-4.13 (m, 2H), 3.82 (d, 2H), 2.83-2.64 (m, 2H), 1.92-1.84 (m, 2H), 1.49-1.43 (m, 10H), 1.38-1.20 (m, 11H), LC-MS (method 4): Rₜ=1.79 min; MS (ESIpos): m/z=not detected

### Intermediate 123

### tert-Butyl 4-[(4-(tert-butyl)-2-sulfamoylphenoxy)methyl]piperidine-1-carboxylate

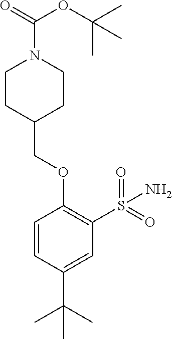 [SMILES: CC(C)(C)OC(=O)N1CCC(COc2ccc(C(C)(C)C)cc2S(N)(=O)=O)CC1]

To a 40 mL vial was added tert-butyl 4-[(4-tert-butyl-2-iodo-phenoxy)methyl]piperidine-1-carboxylate (1.08 g, 2.29 mmol, 1.00 eq.), palladium(II) acetate (25.7 mg, 0.11 mmol, 5.00 mol %), CataCXium® A (65.6 mg, 0.18 mmol, 7.50 mol %), and 1,4-Diazabicyclo[2.2.2]octane bis(sulfur dioxide) adduct (330 mg, 1.37 mmol, 0.60 eq.) sequentially followed by the addition of degassed anhydrous isopropanol (11.4 mL, 0.20 M) and triethylamine (0.95 mL, 6.86 mmol, 3.00 eq.). The resulting red solution was heated at 75° C. for 16 h, cooled to room temperature and concentrated under reduced pressure. The residue was resuspended in anhydrous tetrahydrofuran (12.0 mL) and cooled to 0° C. in a ice/water bath. To this mixture was then added sulfuryl chloride (550 μL, 6.86 mmol, 3.00 eq.) dropwise, after which the resulting mixture was warmed to room temperature and stirred for a further 30 minutes. The reaction was quenched with ammonium chloride (20 mL) and extracted with dichloromethane (3×20 mL). The combined organic extracts were washed with brine (20 mL), dried (magnesium sulfate), filtered and concentrated under reduced pressure. The residue was was resuspended in anhydrous tetrahydrofuran (12.0 mL) and to this solution was added ammonium hydroxide solution (28.0-30.0% NH₃ basis, 6.50 mL, 45.7 mmol, 20.0 eq.). After stirring for a further 30 minutes, the mixture was diluted with aqueous hydrochloric acid (1.00 M, 30 mL) and extracted with ethyl acetate (3×20 mL). The combined organic extracts were washed with brine (20 mL), dried (magnesium sulfate), filtered, and dry loaded onto Celite under reduced pressure. The residue was purified by flash column chromatography (80 g HP Silica, 0-100%, ethyl acetate/hexanes gradient) to give the title compound as a white solid (198 mg). ¹H NMR (400 MHz, Chloroform-d) δ 7.97-7.90 (m, 1H), 7.57-7.49 (m, 1H), 6.99-6.90 (m, 1H), 4.94 (s, 2H), 4.03-3.96 (m, 2H), 2.81-2.70 (m, 2H), 2.09-2.00 (m, 1H), 1.90-1.82 (m, 2H), 1.49-1.42 (m, 10H), 1.36-1.21 (m, 12H), LC-MS (method 4): Rₜ=1.57; MS (ESIpos): m/z=427 [M+H]+

### EXPERIMENTAL SECTION—EXAMPLES

### Example 1

### 6-(Azetidin-1-yl)-N-(2-ethoxy-6-methoxybenzene-1-sulfonyl)-4-fluoro-1-benzofuran-2-carboxamide

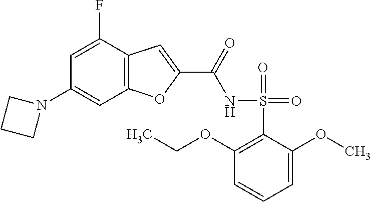 [SMILES: CCOc1cccc(OC)c1S(=O)(=O)NC(=O)c1cc2c(F)cc(N3CCC3)cc2o1]

According to GP5A, 6-(azetidin-1-yl)-4-fluoro-1-benzofuran-2-carboxylic acid

Intermediate 3, 127 mg, 540 μmol), 2-ethoxy-6-methoxybenzene-1-sulfonamide (150 mg, 649 μmol), PyBOP (338 mg, 649 μmol) and DIPEA (380 μL, 2.2 mmol) were stirred at RT in DCM (0.4 mL) overnight. After reaction completion, work-up B and purification using HPLC (acid), the desired compound was obtained as a light yellow solid (75 mg, 29%). ¹H-NMR (400 MHz, DMSO-d₆) δ [ppm]: 1.185 (4.11), 1.202 (9.26), 1.220 (4.22), 2.074 (0.46), 2.311 (1.15), 2.330 (1.77), 2.349 (1.26), 2.366 (0.40), 2.518 (1.04), 2.523 (0.68), 3.783 (16.00), 3.878 (3.51), 3.896 (5.76), 3.915 (3.32), 4.053 (1.02), 4.071 (3.34), 4.088 (3.34), 4.106 (0.98), 6.319 (1.31), 6.323 (1.44), 6.349 (1.21), 6.353 (1.54), 6.375 (2.34), 6.377 (2.80), 6.381 (1.92), 6.766 (4.49), 6.787 (4.70), 7.470 (1.17), 7.491 (2.06), 7.513 (1.00), 8.018 (1.43), 12.135 (2.30); LC-MS (method 7): Rₜ=1.16 min; MS (ESIpos): m/z=449 [M+H]⁺

### Example 2

### 6-(Azetidin-1-yl)-N-(2-ethoxybenzene-1-sulfonyl)-4-fluoro-1-benzofuran-2-carboxamide

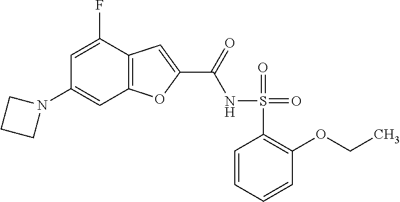 [SMILES: CCOc1ccccc1S(=O)(=O)NC(=O)c1cc2c(F)cc(N3CCC3)cc2o1]

According to GP5A, 6-(azetidin-1-yl)-4-fluoro-1-benzofuran-2-carboxylic acid Intermediate 3, 150 mg, 638 μmol), commercially available 2-ethoxybenzene-1-sulfonamide (CAS: 58734-61-5, 154 mg, 765 μmol) and PyBOP (398 mg, 765 μmol) were suspended in DCM (6.3 mL), then DIPEA (440 μL, 2.6 mmol) was added. The resulting reaction mixture was stirred at RT over night. After reaction completion, work-up B and a first purification using HPLC (acid), the desired compound was obtained as a first fraction as a yellow solid (24.7 mg, 8% yield). And after a second purification as a second other fraction as a light yellow solid (18 mg, 6%). Analytics for the second fraction: ¹H-NMR (400 MHz, DMSO-d₆) δ [ppm]: 1.229 (7.70), 1.246 (16.00), 1.264 (7.73), 2.076 (0.70), 2.290 (0.63), 2.308 (2.01), 2.329 (4.10), 2.334 (2.00), 2.338 (1.29), 2.345 (2.19), 2.363 (0.66), 2.520 (5.98), 2.524 (3.97), 2.542 (0.47), 2.662 (0.59), 2.666 (1.23), 2.671 (1.68), 2.676 (1.21), 2.680 (0.54), 3.874 (6.19), 3.892 (10.15), 3.911 (5.87), 4.137 (1.74), 4.155 (5.56), 4.172 (5.38), 4.189 (1.67), 6.319 (2.10), 6.323 (2.44), 6.353 (6.46), 6.355 (5.92), 7.112 (1.42), 7.130 (2.82), 7.150 (1.55), 7.205 (2.46), 7.225 (2.65), 7.621 (1.02), 7.625 (1.10), 7.643 (1.76), 7.661 (0.90), 7.664 (0.86), 7.890 (2.93), 7.895 (2.90), 7.910 (2.83), 7.914 (2.63), 8.052 (1.52), 12.421 (1.05); LC-MS (method 1): Rₜ=1.26 min; MS (ESIpos): m/z=419 [M+H]⁺.

### Example 3

### 6-(Azetidin-1-yl)-N-[2-(cyclopentyloxy)-5-ethylbenzene-1-sulfonyl]-4-fluoro-1-benzofuran-2-carboxamide

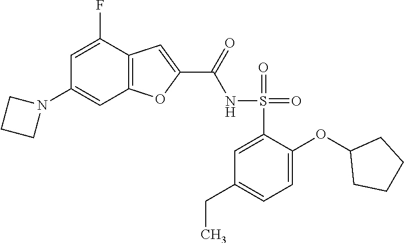 [SMILES: CCc1ccc(OC2CCCC2)c(S(=O)(=O)NC(=O)c2cc3c(F)cc(N4CCC4)cc3o2)c1]

To a stirring suspension of 6-(azetidin-1-yl)-4-fluoro-1-benzofuran-2-carboxylic acid (Intermediate 3, 100 mg, 425 μmol), 2-(cyclopentyloxy)-5-ethylbenzene-1-sulfonamide (137 mg, 510 μmol) and PyBOP (265 mg, 510 μmol; CAS-RN:[128625-52-5]) in dichloromethane (2.1 mL) was added N,N-diisopropylethylamine (300 μL, 1.7 mmol; CAS-RN:[7087-68-5]) and the resulting reaction mixture was stirred at room temperature overnight. After completion the reaction mixture was diluted with water and extracted with dichloromethane twice. The combined organic layers were dried over sodium sulfate, concentrated in vacuo and the residue was purified by HT-HPLC (acid) to give 3.00 mg (75% purity, 1% yield) of the title compound as a white solid. ¹H-NMR (400 MHz, DMSO-d₆) δ [ppm]: 1.135 (1.88), 1.154 (4.54), 1.167 (6.69), 1.173 (2.46), 1.186 (16.00), 1.205 (6.85), 1.418 (1.60), 1.435 (1.25), 1.516 (1.37), 1.533 (2.00), 1.546 (2.23), 1.719 (1.45), 1.804 (1.29), 1.821 (1.68), 2.307 (1.56), 2.317 (1.29), 2.322 (2.78), 2.326 (4.62), 2.332 (2.62), 2.336 (1.33), 2.345 (1.76), 2.518 (7.20), 2.522 (5.32), 2.573 (1.10), 2.592 (1.13), 2.602 (1.25), 2.621 (3.68), 2.640 (3.60), 2.660 (1.84), 2.664 (1.88), 2.668 (2.35), 2.673 (1.68), 2.678 (0.70), 3.875 (4.73), 3.894 (7.82), 3.912 (4.46), 4.920 (1.29), 6.318 (1.76), 6.344 (5.79), 6.608 (0.67), 6.627 (0.63), 7.086 (1.25), 7.093 (1.53), 7.107 (1.45), 7.115 (1.64), 7.371 (0.59), 7.377 (0.55), 7.398 (0.47), 7.458 (1.02), 7.473 (0.94), 7.560 (0.98), 7.566 (0.94), 7.702 (3.17), 7.708 (2.97), 8.028 (1.45), 12.254 (1.37); LC-MS (method 3): Rₜ=1.45 min; MS (ESIpos): m/z=487 [M+H]⁺

### Example 4

### 6-(Azetidin-1-yl)-N-(2-{[butan-2-yl]oxy}-5-methylbenzene-1-sulfonyl)-4-fluoro-1-benzofuran-2-carboxamide

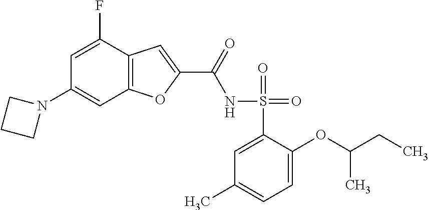 [SMILES: CCC(C)Oc1ccc(C)cc1S(=O)(=O)NC(=O)c1cc2c(F)cc(N3CCC3)cc2o1]

Synthesized and purified analogously to Example 3 using 2-(butan-2-yloxy)-5-methylbenzene-1-sulfonamide (124 mg, 510 μmol) instead of 2-(cyclopentyloxy)-5-ethylbenzene-1-sulfonamide to give the title compound as a white solid (40 mg, 19% yield). ¹H-NMR (400 MHz, DMSO-d₆) δ [ppm]: 0.762 (4.57), 0.780 (10.83), 0.798 (5.01), 1.074 (10.44), 1.089 (10.43), 1.301 (0.78), 1.316 (0.79), 1.472 (0.53), 1.485 (0.67), 1.490 (0.67), 1.506 (1.05), 1.520 (0.85), 1.524 (0.84), 1.538 (0.70), 1.649 (0.73), 1.667 (1.15), 1.684 (1.16), 1.702 (0.87), 1.718 (0.49), 2.286 (0.62), 2.314 (16.00), 2.341 (2.07), 2.359 (0.64), 2.518 (2.98), 2.522 (1.88), 2.664 (0.53), 2.669 (0.74), 2.673 (0.54), 3.367 (0.58), 3.372 (0.54), 3.376 (0.41), 3.841 (0.53), 3.860 (1.07), 3.871 (5.03), 3.878 (1.89), 3.889 (8.38), 3.907 (4.77), 4.508 (0.58), 4.523 (1.04), 4.538 (1.04), 4.553 (0.55), 6.309 (1.66), 6.313 (1.90), 6.344 (5.56), 7.087 (0.52), 7.090 (0.53), 7.110 (1.52), 7.131 (1.71), 7.405 (1.21), 7.425 (1.04), 7.692 (3.04), 7.696 (2.88), 8.041 (1.42), 12.256 (1.46); LC-MS (method 7): Rₜ=1.39 min; MS (ESIpos): m/z=461 [M+H]⁺. Since the stereochemistry was unsure, an optical rotation was performed:

Specific Rotation: +2.2° (DMSO, 20° C., 589 nm)

### Example 5

### 6-(Azetidin-1-yl)-N-[2-(benzyloxy)-6-(cyclobutyloxy)benzene-1-sulfonyl]-4-fluoro-1-benzofuran-2-carboxamide

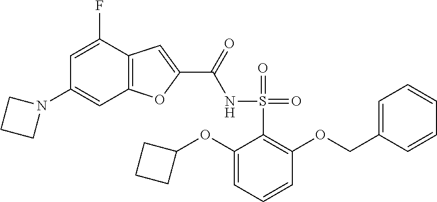 [SMILES: O=C(NS(=O)(=O)c1c(OCc2ccccc2)cccc1OC1CCC1)c1cc2c(F)cc(N3CCC3)cc2o1]

Synthesized and purified analogously to Example 3 using 2-(benzyloxy)-6-(cyclobutyloxy)benzene-1-sulfonamide (210 mg, 630 μmol) instead of 2-(cyclopentyloxy)-5-ethylbenzene-1-sulfonamide to yield 43.0 mg (13% yield) of the title compound as an ochre solid. ¹H-NMR (400 MHz, DMSO-d₆) δ [ppm]: 1.444 (1.42), 1.462 (2.07), 1.470 (2.50), 1.481 (3.24), 1.500 (1.92), 1.505 (1.97), 1.524 (0.75), 1.912 (0.73), 1.919 (0.60), 1.937 (2.38), 1.943 (1.96), 1.955 (2.24), 1.961 (3.34), 1.967 (2.46), 1.979 (1.80), 1.986 (2.50), 2.011 (0.75), 2.200 (0.92), 2.209 (1.35), 2.218 (2.11), 2.226 (2.82), 2.234 (2.60), 2.242 (2.62), 2.249 (2.35), 2.259 (1.90), 2.266 (1.11), 2.276 (0.77), 2.296 (1.11), 2.314 (3.31), 2.322 (1.97), 2.332 (4.91), 2.352 (3.55), 2.369 (1.16), 2.518 (2.59), 2.523 (1.74), 2.664 (0.60), 2.669 (0.85), 2.673 (0.58), 3.885 (9.68), 3.904 (16.00), 3.922 (9.11), 4.696 (0.58), 4.714 (2.30), 4.732 (3.40), 4.750 (2.17), 4.767 (0.52), 5.246 (15.52), 6.321 (3.62), 6.326 (3.95), 6.351 (3.63), 6.355 (4.33), 6.380 (8.02), 6.551 (4.90), 6.572 (5.04), 6.819 (4.65), 6.841 (4.91), 7.224 (0.58), 7.228 (0.56), 7.249 (1.24), 7.259 (0.94), 7.267 (4.54), 7.274 (1.39), 7.281 (2.83), 7.285 (4.85), 7.289 (2.59), 7.299 (7.63), 7.303 (3.53), 7.314 (4.38), 7.318 (10.35), 7.331 (1.59), 7.335 (3.72), 7.339 (2.56), 7.408 (3.16), 7.429 (5.74), 7.450 (2.85), 7.495 (7.78), 7.511 (6.57), 7.515 (4.99), 7.987 (5.86), 12.217 (6.04); LC-MS (method 3): Rₜ=1.41 min; MS (ESIpos): m/z=551 [M+H]⁺

### Example 6

### 6-(Azetidin-1-yl)-N-(2-ethoxy-5-methylbenzene-1-sulfonyl)-4-fluoro-1-benzofuran-2-carboxamide

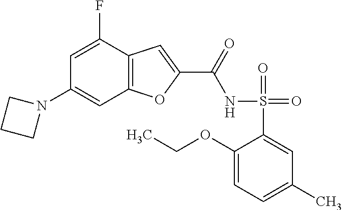 [SMILES: CCOc1ccc(C)cc1S(=O)(=O)NC(=O)c1cc2c(F)cc(N3CCC3)cc2o1]

Synthesized and purified analogously to Example 3 using commercially available 2-ethoxy-5-methylbenzene-1-sulfonamide (CAS: 187471-14-3, 110 mg, 510 μmol) instead of 2-(cyclopentyloxy)-5-ethylbenzene-1-sulfonamide to give 6.00 mg (3% yield) of the title compound as a green solid. ¹H-NMR (400 MHz, DMSO-d₆) δ [ppm]: 1.201 (5.20), 1.218 (12.42), 1.236 (5.72), 2.287 (0.53), 2.306 (1.58), 2.324 (16.00), 2.342 (2.28), 2.360 (0.81), 2.518 (6.85), 2.522 (4.94), 2.539 (2.43), 2.669 (0.56), 2.673 (0.43), 3.872 (4.35), 3.890 (7.31), 3.908 (4.50), 4.087 (1.39), 4.104 (4.52), 4.122 (4.65), 4.139 (1.49), 6.315 (1.56), 6.319 (1.99), 6.348 (5.30), 7.100 (2.68), 7.121 (3.00), 7.430 (1.32), 7.436 (1.42), 7.452 (1.24), 7.457 (1.30), 7.696 (2.66), 7.702 (2.72), 8.048 (2.43), 12.380 (0.96); LC-MS (method 6): Rₜ=1.28 min; MS (ESIpos): m/z=433 [M+H]⁺

### Example 7

### 6-(Azetidin-1-yl)-N-[2-(benzyloxy)-6-ethoxybenzene-1-sulfonyl]-4-fluoro-1-benzofuran-2-carboxamide

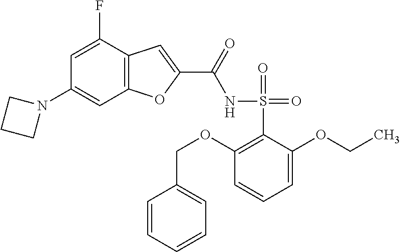 [SMILES: CCOc1cccc(OCc2ccccc2)c1S(=O)(=O)NC(=O)c1cc2c(F)cc(N3CCC3)cc2o1]

Synthesized and purified analogously to Example 3 using 2-(benzyloxy)-6-ethoxybenzene-1-sulfonamide (150 mg, 488 μmol) instead of 2-(cyclopentyloxy)-5-ethylbenzene-1-sulfonamide to give 75.0 mg (33% yield) of the title compound as a light yellow solid. ¹H-NMR (400 MHz, DMSO-d₆) δ [ppm]: 1.158 (7.08), 1.175 (16.00), 1.192 (7.35), 1.302 (1.74), 1.317 (1.77), 2.074 (0.43), 2.298 (0.89), 2.317 (2.81), 2.334 (3.85), 2.352 (2.67), 2.370 (0.85), 2.522 (3.82), 2.539 (0.85), 2.665 (0.71), 2.669 (0.97), 2.673 (0.70), 3.842 (1.02), 3.860 (1.73), 3.884 (6.63), 3.903 (11.18), 3.921 (6.33), 4.046 (1.79), 4.063 (5.66), 4.080 (5.75), 4.098 (1.80), 5.241 (10.27), 6.321 (2.39), 6.324 (2.53), 6.354 (2.68), 6.372 (5.87), 6.394 (0.71), 6.757 (3.13), 6.778 (3.34), 6.813 (3.11), 6.834 (3.32), 7.089 (1.06), 7.091 (0.98), 7.233 (0.63), 7.241 (0.43), 7.250 (2.51), 7.258 (0.96), 7.267 (3.73), 7.276 (5.46), 7.294 (6.62), 7.306 (1.16), 7.311 (2.12), 7.316 (1.60), 7.437 (1.74), 7.459 (3.24), 7.474 (5.52), 7.491 (4.40), 7.961 (3.85), 12.176 (4.60); LC-MS (method 7): Rₜ=1.36 min; MS (ESIpos): m/z=525 [M+H]⁺

### Example 8

### 6-(Azetidin-1-yl)-4-fluoro-N-(7-methoxyquinoline-8-sulfonyl)-1-benzofuran-2-carboxamide

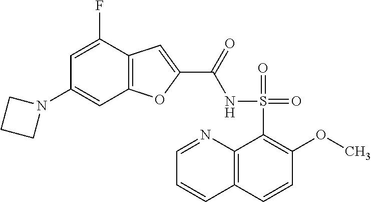 [SMILES: COc1ccc2cccnc2c1S(=O)(=O)NC(=O)c1cc2c(F)cc(N3CCC3)cc2o1]

Synthesized and purified analogously to Example 3 using commercially available 7-methoxyquinoline-8-sulfonamide (CAS: 2059988-75-7, 122 mg, 510 μmol) instead of 2-(cyclopentyloxy)-5-ethylbenzene-1-sulfonamide to give 2.00 mg (85% purity, 1% yield) of the title compound as an orange solid. ¹H-NMR (400 MHz, DMSO-d₆) δ [ppm]: 1.230 (0.80), 1.727 (1.03), 2.074 (0.71), 2.295 (1.18), 2.314 (1.75), 2.322 (0.93), 2.327 (1.10), 2.331 (1.70), 2.518 (5.74), 2.523 (3.99), 2.535 (1.68), 2.539 (1.36), 2.544 (1.05), 2.669 (0.47), 3.004 (0.76), 3.013 (0.77), 3.847 (3.46), 3.866 (5.96), 3.884 (3.58), 4.053 (16.00), 6.269 (1.35), 6.273 (1.62), 6.298 (1.31), 6.302 (1.68), 6.326 (2.31), 6.328 (2.81), 6.332 (2.08), 7.678 (3.40), 7.679 (3.70), 7.681 (3.10), 7.694 (1.74), 7.702 (1.75), 7.714 (1.72), 7.815 (2.51), 7.838 (2.74), 8.387 (2.72), 8.410 (2.54), 8.729 (1.33), 8.733 (1.46), 8.749 (1.36), 8.753 (1.42), 9.096 (1.61), 9.100 (1.75), 9.108 (1.73), 9.112 (1.65); LC-MS (method 3): Rₜ=0.86 min; MS (ESIpos): m/z=456 [M+H]⁺

### Example 9

### 6-(Azetidin-1-yl)-N-[2-(cyclopentyloxy)-5-methylbenzene-1-sulfonyl]-4-fluoro-1-benzofuran-2-carboxamide

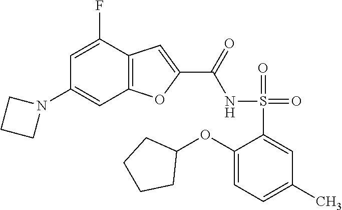 [SMILES: Cc1ccc(OC2CCCC2)c(S(=O)(=O)NC(=O)c2cc3c(F)cc(N4CCC4)cc3o2)c1]

Synthesized and purified analogously to Example 3 using commercially available 2-(cyclopentyloxy)-5-methylbenzene-1-sulfonamide (CAS: 1094691-37-8, 130 mg, 510 μmol) instead of 2-(cyclopentyloxy)-5-ethylbenzene-1-sulfonamide to give 48.0 mg (95% purity, 23% yield) of the title compound as a mint solid. ¹H-NMR (400 MHz, DMSO-d₆) δ [ppm]: 1.392 (0.80), 1.401 (1.25), 1.413 (1.67), 1.430 (1.30), 1.455 (0.49), 1.484 (0.43), 1.511 (1.22), 1.522 (1.18), 1.529 (1.60), 1.541 (1.41), 1.548 (0.93), 1.556 (0.59), 1.665 (0.68), 1.677 (0.82), 1.693 (1.13), 1.699 (1.29), 1.706 (1.26), 1.713 (1.35), 1.784 (0.55), 1.801 (1.21), 1.817 (1.47), 1.835 (1.15), 1.851 (0.70), 2.289 (0.60), 2.316 (16.00), 2.326 (3.53), 2.337 (0.97), 2.344 (1.89), 2.363 (0.59), 2.518 (2.39), 2.523 (1.70), 2.665 (0.55), 2.669 (0.78), 2.673 (0.54), 3.875 (5.05), 3.893 (8.32), 3.912 (4.75), 4.900 (0.69), 4.907 (1.03), 4.915 (1.30), 4.922 (1.01), 4.930 (0.69), 6.313 (1.65), 6.317 (1.99), 6.345 (9.07), 7.069 (1.86), 7.090 (2.08), 7.415 (1.17), 7.433 (1.03), 7.689 (2.93), 7.694 (2.74), 8.026 (1.41), 12.251 (1.41); LC-MS (method 7): Rₜ=1.41 min; MS (ESIpos): m/z=473 [M+H]⁺

### Example 10

### 6-(Azetidin-1-yl)-N-(2-ethoxy-6-propoxybenzene-1-sulfonyl)-4-fluoro-1-benzofuran-2-carboxamide

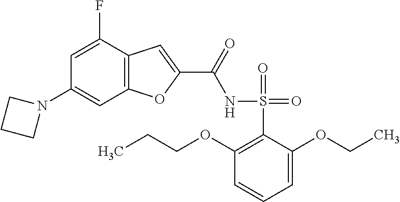 [SMILES: CCCOc1cccc(OCC)c1S(=O)(=O)NC(=O)c1cc2c(F)cc(N3CCC3)cc2o1]

Synthesized and purified analogously to Example 3 using 2-ethoxy-6-propoxybenzene-1-sulfonamide (150 mg, 578 μmol) instead of 2-(cyclopentyloxy)-5-ethylbenzene-1-sulfonamide to give 83 mg (34% yield) of the title compound as a yellow solid. ¹H-NMR (400 MHz, DMSO-d₆) δ [ppm]: 0.916 (7.26), 0.934 (15.76), 0.953 (7.98), 1.146 (7.77), 1.163 (16.00), 1.181 (7.96), 1.209 (1.95), 1.225 (1.93), 1.628 (0.68), 1.645 (2.87), 1.662 (5.48), 1.679 (5.34), 1.696 (2.78), 1.713 (0.77), 2.295 (1.17), 2.312 (4.04), 2.330 (6.50), 2.348 (3.90), 2.366 (1.23), 2.665 (1.57), 3.849 (0.75), 3.880 (9.11), 3.898 (15.64), 3.917 (8.27), 3.972 (4.48), 3.988 (8.64), 4.004 (4.33), 4.028 (2.47), 4.046 (6.88), 4.063 (6.78), 4.080 (2.32), 6.321 (4.02), 6.350 (3.94), 6.376 (8.65), 6.407 (0.59), 6.729 (4.21), 6.752 (6.13), 6.778 (4.33), 7.438 (2.23), 7.459 (4.02), 7.480 (1.96), 8.000 (6.61), 12.071 (6.79); LC-MS (method 7): Rₜ=1.31 min; MS (ESIpos): m/z=477 [M+H]⁺

### Example 11

### 6-(Azetidin-1-yl)-N-[2-(dimethylamino)benzene-1-sulfonyl]-4-fluoro-1-benzofuran-2-carboxamide

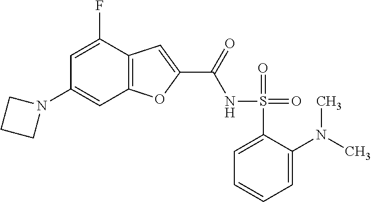 [SMILES: CN(C)c1ccccc1S(=O)(=O)NC(=O)c1cc2c(F)cc(N3CCC3)cc2o1]

Synthesized and purified analogously to Example 3 using commercially available 2-(dimethylamino)benzene-1-sulfonamide (CAS: 98489-78-2, 102 mg, 510 μmol) instead of 2-(cyclopentyloxy)-5-ethylbenzene-1-sulfonamide to give 7.00 mg (4% yield) of the title compound as a yellow solid. ¹H-NMR (400 MHz, DMSO-d₆) δ [ppm]: 2.296 (0.96), 2.314 (1.43), 2.322 (0.63), 2.327 (0.72), 2.332 (1.27), 2.350 (0.41), 2.518 (2.96), 2.523 (2.10), 2.539 (0.63), 3.113 (16.00), 3.340 (0.67), 3.845 (2.90), 3.863 (4.92), 3.881 (2.93), 6.257 (1.16), 6.261 (1.29), 6.286 (1.15), 6.290 (1.29), 6.352 (1.76), 6.355 (2.23), 6.359 (1.79), 7.512 (2.81), 7.514 (2.98), 7.526 (0.70), 7.528 (0.71), 7.547 (1.26), 7.564 (0.77), 7.567 (0.77), 7.728 (0.58), 7.732 (0.64), 7.749 (1.03), 7.753 (1.01), 7.768 (0.70), 7.771 (0.71), 7.861 (1.25), 7.881 (0.92), 7.932 (1.59), 7.936 (1.66), 7.952 (1.50), 7.956 (1.45); LC-MS (method 3): Rₜ=0.98 min; MS (ESIpos): m/z=418 [M+H]⁺

### Example 12

### 6-(Azetidin-1-yl)-N-(3,4-dihydro-2H-1-benzopyran-8-sulfonyl)-4-fluoro-1-benzofuran-2-carboxamide

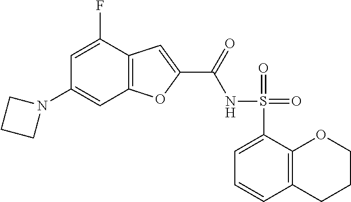 [SMILES: O=C(NS(=O)(=O)c1cccc2c1OCCC2)c1cc2c(F)cc(N3CCC3)cc2o1]

Synthesized and purified analogously to Example 3 using 3,4-dihydro-2H-1-benzopyran-8-sulfonamide (109 mg, 510 μmol) instead of 2-(cyclopentyloxy)-5-ethylbenzene-1-sulfonamide to give 3.00 mg (2% yield) of the title compound as a yellow solid. ¹H-NMR (400 MHz, DMSO-d₆) δ [ppm]: 1.872 (3.42), 1.885 (4.55), 1.898 (3.61), 2.290 (1.09), 2.309 (3.48), 2.327 (10.03), 2.345 (4.16), 2.363 (1.27), 2.665 (3.57), 2.669 (5.04), 2.758 (3.46), 2.774 (6.54), 2.790 (3.36), 3.873 (9.05), 3.891 (16.00), 3.910 (9.08), 4.198 (4.23), 4.212 (6.28), 4.224 (4.62), 6.320 (3.94), 6.349 (4.16), 6.363 (8.32), 6.980 (2.21), 6.999 (4.69), 7.018 (2.64), 7.371 (3.04), 7.390 (2.80), 7.684 (3.89), 7.704 (3.65), 8.032 (2.68), 12.442 (2.21); LC-MS (method 3): Rₜ=1.21 min; MS (ESIpos): m/z=431 [M+H]⁺

### Example 13

### 6-(Azetidin-1-yl)-4-fluoro-N-(2-{[-1,1,1-trifluoropropan-2-yl]oxy}benzene-1-sulfonyl)-1-benzofuran-2-carboxamide (rac)

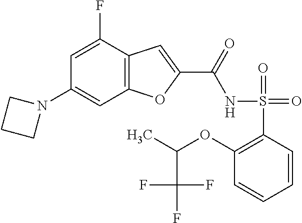 [SMILES: CC(Oc1ccccc1S(=O)(=O)NC(=O)c1cc2c(F)cc(N3CCC3)cc2o1)C(F)(F)F]

Synthesized an purified analogously to Example 3 using commercially available 2-[(1,1,1-trifluoropropan-2-yl)oxy]benzenesulfonamide (CAS: 1192258-44-8, 72.0 mg, 267 μmol) instead of 2-(cyclopentyloxy)-5-ethylbenzene-1-sulfonamide to give 20.0 mg (18% yield) of the title compound as yellow solid. ¹H-NMR (400 MHz, DMSO-d₆) δ [ppm]: 1.169 (0.79), 1.210 (2.40), 1.226 (2.45), 1.372 (13.16), 1.387 (13.32), 2.074 (1.47), 2.286 (1.06), 2.304 (3.36), 2.322 (5.34), 2.341 (3.74), 2.359 (1.21), 2.518 (4.14), 2.522 (2.73), 2.539 (0.81), 2.664 (0.55), 2.669 (0.75), 2.673 (0.55), 3.849 (0.80), 3.871 (9.49), 3.890 (15.66), 3.908 (8.96), 5.503 (0.74), 5.519 (1.84), 5.535 (2.42), 5.551 (1.78), 5.567 (0.71), 6.313 (3.44), 6.317 (4.16), 6.345 (16.00), 6.407 (0.41), 7.216 (2.52), 7.218 (2.60), 7.236 (5.09), 7.254 (2.83), 7.256 (2.85), 7.440 (3.55), 7.462 (4.20), 7.672 (1.97), 7.676 (2.13), 7.695 (3.16), 7.712 (1.62), 7.716 (1.58), 7.952 (4.84), 7.957 (4.89), 7.972 (4.71), 7.976 (4.52), 8.001 (3.81), 12.487 (0.83); LC-MS (method 7): Rₜ=1.27 min; MS (ESIpos): m/z=487 [M+H]⁺

### Example 14

### Enantiomer 1 of Example 13

Chiral separation of

Example 13 using PrepCon Labomatic HPLC-3; Column: Chiralpak IG 5 μm, 250×30; eluent A: hexane+0.1 vol % trifluoracetic acid; eluent B: 2-propanol; isocratic: 60% A+40% B; flow: 40 ml/min; temperature: 25° C.; UV: 254 nm to give 14 as light yellow solid. ¹H-NMR (400 MHz, DMSO-d₆) δ [ppm]: 1.137 (0.41), 1.233 (1.54), 1.256 (0.99), 1.296 (0.56), 1.363 (5.26), 1.379 (5.30), 2.286 (0.60), 2.304 (1.97), 2.323 (6.03), 2.327 (6.06), 2.332 (3.93), 2.337 (2.41), 2.341 (2.34), 2.359 (0.64), 2.518 (16.00), 2.523 (10.59), 2.660 (1.40), 2.665 (3.23), 2.669 (4.54), 2.673 (3.09), 2.679 (1.40), 3.868 (4.64), 3.886 (7.92), 3.905 (4.41), 6.309 (1.08), 6.346 (5.69), 7.225 (1.02), 7.681 (0.64), 7.943 (1.34), 7.961 (1.32), 12.481 (0.53); LC-MS method 7): Rₜ=1.27 min; MS (ESIpos): m/z=487 [M+H]⁺

Specific Rotation: not applicable (DMSO, 20° C., 589 nm)

### Example 15

### Enantiomer 2 of Example 13

Chiral separation of

Example 13 using PrepCon Labomatic HPLC-3; Column: Chiralpak IG 5 μm, 250×30; eluent A: hexane+0.1 vol % trifluoracetic acid; eluent B: 2-propanol; isocratic: 60% A+40% B; flow: 40 mL/min; temperature: 25° C.; UV: 254 nm to give 15 as light yellow solid. ¹H-NMR (400 MHz, DMSO-d₆) δ [ppm]: 1.137 (0.41), 1.233 (1.54), 1.256 (0.99), 1.296 (0.56), 1.363 (5.26), 1.379 (5.30), 2.286 (0.60), 2.304 (1.97), 2.323 (6.03), 2.327 (6.06), 2.332 (3.93), 2.337 (2.41), 2.341 (2.34), 2.359 (0.64), 2.518 (16.00), 2.523 (10.59), 2.660 (1.40), 2.665 (3.23), 2.669 (4.54), 2.673 (3.09), 2.679 (1.40), 3.868 (4.64), 3.886 (7.92), 3.905 (4.41), 6.309 (1.08), 6.346 (5.69), 7.225 (1.02), 7.681 (0.64), 7.943 (1.34), 7.961 (1.32), 12.481 (0.53); LC-MS method 7): Rₜ=1.27 min; MS (ESIpos): m/z=487 [M+H]⁺

Specific Rotation: +11° (DMSO, 20° C., 589 nm)

### Example 16

### 6-(Azetidin-1-yl)-N-[2-(cyclobutyloxy)-6-fluorobenzene-1-sulfonyl]-4-fluoro-1-benzofuran-2-carboxamide

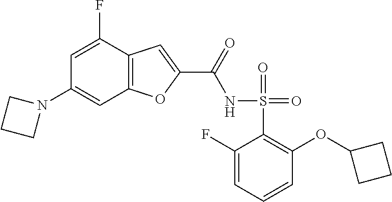 [SMILES: O=C(NS(=O)(=O)c1c(F)cccc1OC1CCC1)c1cc2c(F)cc(N3CCC3)cc2o1]

Synthesized and purified analogously to Example 3 using 2-(cyclobutyloxy)-6-fluorobenzene-1-sulfonamide (125 mg, 510 μmol) instead of 2-(cyclopentyloxy)-5-ethylbenzene-1-sulfonamide to give 63.0 mg (29% yield) of the title compound as an ochre solid. ¹H-NMR (400 MHz, DMSO-d₆) δ [ppm]: 1.152 (0.45), 1.169 (0.98), 1.187 (0.47), 1.209 (2.95), 1.227 (3.07), 1.422 (0.73), 1.430 (1.39), 1.437 (0.91), 1.447 (1.29), 1.456 (2.08), 1.464 (2.13), 1.469 (1.47), 1.484 (1.58), 1.489 (2.71), 1.510 (1.42), 1.513 (1.58), 1.534 (0.76), 1.541 (0.56), 1.926 (0.72), 1.933 (0.59), 1.951 (2.41), 1.958 (1.95), 1.969 (2.17), 1.976 (3.37), 1.982 (2.38), 1.994 (1.79), 2.000 (2.49), 2.019 (0.53), 2.026 (0.72), 2.247 (0.95), 2.254 (1.42), 2.267 (1.97), 2.271 (2.64), 2.279 (2.61), 2.285 (2.38), 2.292 (3.23), 2.298 (2.98), 2.303 (2.39), 2.311 (3.86), 2.322 (2.80), 2.331 (5.25), 2.340 (1.54), 2.349 (3.51), 2.367 (1.10), 2.518 (3.21), 2.523 (2.02), 2.664 (0.60), 2.669 (0.85), 2.673 (0.60), 3.850 (0.87), 3.868 (1.50), 3.884 (9.94), 3.902 (16.00), 3.920 (9.31), 4.792 (0.56), 4.810 (2.23), 4.827 (3.34), 4.845 (2.14), 4.863 (0.53), 6.328 (3.64), 6.332 (4.25), 6.357 (3.01), 6.361 (5.38), 6.367 (7.36), 6.370 (8.12), 6.373 (4.75), 6.408 (0.48), 6.829 (4.15), 6.851 (4.34), 6.947 (2.08), 6.966 (2.48), 6.974 (2.35), 6.994 (2.32), 7.561 (1.45), 7.577 (1.69), 7.582 (2.90), 7.597 (2.85), 7.604 (1.66), 7.619 (1.41), 8.057 (4.68), 12.636 (0.70); LC-MS (method 3): Rₜ=1.28 min; MS (ESIpos): m/z=463 [M+H]⁺

### Example 17

### 6-(Azetidin-1-yl)-N-(2,6-dipropoxybenzene-1-sulfonyl)-4-fluoro-1-benzofuran-2-carboxamide

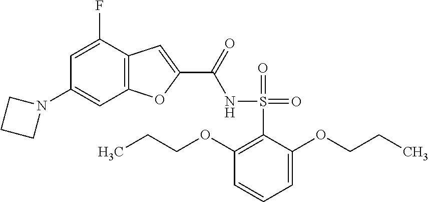 [SMILES: CCCOc1cccc(OCCC)c1S(=O)(=O)NC(=O)c1cc2c(F)cc(N3CCC3)cc2o1]

Synthesized and purified analogously to Example 3 using 2,6-dipropoxybenzene-1-sulfonamide (150 mg, 549 μmol) instead of 2-(cyclopentyloxy)-5-ethylbenzene-1-sulfonamide to give 35.0 mg (15% yield) of the title compound as an offwhite solid. ¹H-NMR (400 MHz, DMSO-d₆) δ [ppm]: 0.875 (6.94), 0.894 (16.00), 0.912 (7.35), 1.596 (0.44), 1.613 (1.75), 1.630 (3.53), 1.648 (3.44), 1.665 (1.62), 2.313 (0.91), 2.322 (0.68), 2.332 (1.61), 2.351 (0.98), 2.518 (1.61), 2.522 (1.06), 2.669 (0.45), 3.880 (2.71), 3.899 (4.44), 3.917 (2.57), 3.954 (2.72), 3.970 (5.55), 3.986 (2.65), 6.318 (0.90), 6.322 (1.00), 6.347 (0.85), 6.351 (1.08), 6.368 (1.92), 6.370 (2.22), 6.741 (2.90), 6.763 (3.09), 7.436 (0.70), 7.457 (1.24), 7.478 (0.60), 7.996 (1.71), 12.039 (1.97); LC-MS method 7): Rₜ=1.38 min; MS (ESIpos): m/z=491 [M+H]⁺

### Example 18

### 6-(Azetidin-1-yl)-4-fluoro-N-[2-methyl-2,3-dihydro-1-benzofuran-7-sulfonyl]-1-benzofuran-2-carboxamide

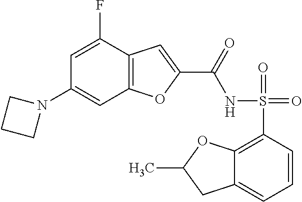 [SMILES: CC1Cc2cccc(S(=O)(=O)NC(=O)c3cc4c(F)cc(N5CCC5)cc4o3)c2O1]

Synthesized and purified analogously to Example 3 using commercially available 2-methyl-2,3-dihydro-1-benzofuran-7-sulfonamide (CAS: 89819-23-8, 109 mg, 510 μmol) instead of 2-(cyclopentyloxy)-5-ethylbenzene-1-sulfonamide to give 49.0 mg (25% yield) of the title compound as a yellow solid. ¹H-NMR (400 MHz, DMSO-d₆) δ [ppm]: 1.236 (15.20), 1.251 (15.92), 1.267 (1.09), 2.074 (16.00), 2.285 (0.90), 2.303 (2.84), 2.321 (4.35), 2.331 (1.61), 2.340 (3.34), 2.357 (1.19), 2.522 (2.37), 2.539 (1.23), 2.779 (1.51), 2.795 (1.58), 2.819 (1.76), 2.836 (1.80), 3.338 (2.40), 3.360 (3.62), 3.378 (2.68), 3.400 (1.92), 3.870 (8.45), 3.889 (14.34), 3.906 (8.62), 5.038 (1.17), 5.054 (1.86), 5.060 (1.51), 5.071 (1.45), 5.076 (1.99), 5.092 (1.25), 6.309 (3.11), 6.312 (3.83), 6.338 (2.66), 6.342 (4.69), 6.349 (6.35), 6.351 (7.24), 6.975 (3.92), 6.994 (5.24), 7.013 (4.23), 7.490 (3.24), 7.492 (3.51), 7.508 (3.14), 7.511 (3.31), 7.589 (3.50), 7.591 (3.63), 7.609 (3.42), 7.612 (3.35), 8.025 (7.88), 12.591 (0.76); LC-MS (method 3): Rₜ=1.21 min; MS (ESIpos): m/z=431 [M+H]⁺

### Example 19

### 6-(Azetidin-1-yl)-N-([1,1′-biphenyl]-2-sulfonyl)-4-fluoro-1-benzofuran-2-carboxamide

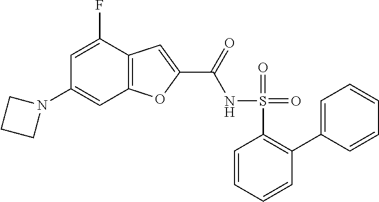 [SMILES: O=C(NS(=O)(=O)c1ccccc1-c1ccccc1)c1cc2c(F)cc(N3CCC3)cc2o1]

Synthesized and purified analogously to Example 3 using commercially available [1,1′-biphenyl]-2-sulfonamide (CAS: 40182-06-7, 326 mg, 1.40 mmol) instead of 2-(cyclopentyloxy)-5-ethylbenzene-1-sulfonamide to give 50.0 mg (9% yield) of the title compound as a yellow solid. ¹H-NMR (400 MHz, DMSO-d₆) δ [ppm]: 2.075 (2.06), 2.295 (1.03), 2.313 (3.23), 2.322 (3.91), 2.327 (5.91), 2.332 (7.42), 2.351 (3.43), 2.368 (1.03), 2.518 (12.09), 2.523 (8.31), 2.539 (1.92), 2.660 (1.24), 2.664 (2.75), 2.669 (3.91), 2.673 (2.82), 2.678 (1.24), 3.879 (9.68), 3.898 (16.00), 3.916 (9.06), 6.307 (3.09), 6.312 (3.57), 6.341 (12.91), 6.343 (11.19), 6.347 (4.67), 7.245 (3.16), 7.249 (4.33), 7.266 (8.65), 7.286 (3.91), 7.289 (5.22), 7.307 (9.89), 7.310 (4.46), 7.321 (5.08), 7.325 (5.15), 7.344 (2.20), 7.348 (3.23), 7.359 (1.37), 7.366 (3.36), 7.383 (1.03), 7.589 (1.03), 7.632 (1.24), 7.651 (2.61), 7.668 (1.92), 7.701 (1.79), 7.719 (2.40), 7.735 (1.03), 8.147 (4.39), 8.150 (4.60), 8.167 (3.98), 8.170 (3.91); LC-MS (method 7): Rₜ=1.34 min; MS (ESIpos): m/z=451 [M+H]⁺

### Example 20 Sodium {[6-(azetidin-1-yl)-4-fluoro-1-benzofuran-2-yl]carbonyl}([biphenyl]-2-ylsulfonyl)azanide

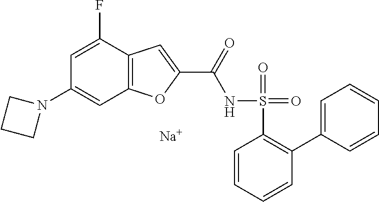 [SMILES: O=C(NS(=O)(=O)c1ccccc1-c1ccccc1)c1cc2c(F)cc(N3CCC3)cc2o1 ; [Na+]]

A solution of 6-(azetidin-1-yl)-N-([1,1′-biphenyl]-2-sulfonyl)-4-fluoro-1-benzofuran-2-carboxamide (Intermediate 19, 140 mg, 311 μmol) in dichloromethane (5 mL) was washed with aq. sodium hydroxide solution (1 M). The organic layer was dried over sodium sulfate and concentrated in vacuo to give 145 mg (98% yield) fo the title compound as a white solid. ¹H-NMR (400 MHz, DMSO-d₆) δ [ppm]: 2.270 (0.99), 2.287 (3.17), 2.306 (4.55), 2.318 (1.46), 2.325 (3.91), 2.332 (1.36), 2.342 (1.11), 2.518 (3.15), 2.523 (2.24), 2.665 (0.74), 2.669 (1.06), 2.673 (0.74), 3.822 (9.74), 3.840 (16.00), 3.851 (2.10), 3.858 (9.04), 5.759 (14.41), 6.187 (3.79), 6.191 (3.93), 6.216 (3.77), 6.221 (3.74), 6.323 (5.73), 6.325 (7.05), 6.329 (5.18), 6.741 (12.85), 6.743 (11.67), 7.102 (3.79), 7.106 (3.15), 7.109 (2.64), 7.119 (5.07), 7.124 (4.19), 7.192 (0.95), 7.196 (2.13), 7.198 (1.96), 7.200 (2.33), 7.203 (8.78), 7.208 (5.44), 7.212 (11.38), 7.219 (13.74), 7.224 (2.36), 7.229 (1.60), 7.380 (1.32), 7.384 (1.83), 7.393 (1.09), 7.398 (4.88), 7.404 (7.96), 7.407 (4.37), 7.410 (4.26), 7.413 (12.11), 7.416 (9.22), 7.419 (8.21), 7.421 (9.69), 7.424 (4.85), 7.428 (5.81), 7.431 (5.25), 7.436 (4.46), 7.450 (1.59), 7.454 (1.07), 8.050 (4.30), 8.055 (5.25), 8.067 (3.01), 8.070 (3.24), 8.074 (3.88); LC-MS (method 7): Rₜ=1.34 min; MS (ESIpos): m/z=451 [M+H]⁺

### Example 21

### 6-(Azetidin-1-yl)-N-(2,2-dimethyl-2,3-dihydro-1-benzofuran-7-sulfonyl)-4-fluoro-1-benzofuran-2-carboxamide

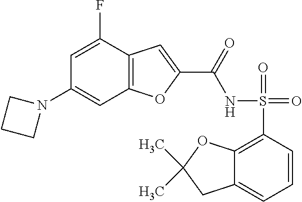 [SMILES: CC1(C)Cc2cccc(S(=O)(=O)NC(=O)c3cc4c(F)cc(N5CCC5)cc4o3)c2O1]

Synthesized and purified analogously to Example 3 using commercially available 2,2-dimethyl-2,3-dihydro-1-benzofuran-7-sulfonamide (CAS: 87254-53-3, 116 mg, 510 μmol) instead of 2-(cyclopentyloxy)-5-ethylbenzene-1-sulfonamide to give 57.0 mg (27% yield) of the title compound as a yellow solid. ¹H-NMR (400 MHz, DMSO-d₆) δ [ppm]: 1.209 (0.84), 1.226 (0.82), 1.304 (16.00), 2.302 (0.84), 2.321 (1.27), 2.339 (0.94), 3.028 (3.16), 3.351 (0.43), 3.869 (2.54), 3.887 (4.01), 3.906 (2.34), 6.308 (0.91), 6.312 (1.09), 6.337 (0.76), 6.341 (1.32), 6.349 (1.78), 6.351 (1.98), 6.966 (1.14), 6.984 (1.48), 6.986 (1.53), 7.005 (1.27), 7.473 (0.96), 7.476 (1.01), 7.491 (0.91), 7.494 (0.90), 7.583 (1.05), 7.586 (1.05), 7.603 (1.01), 7.606 (0.93), 8.038 (1.75); LC-MS (method 3): Rₜ=1.26 min; MS (ESIpos): m/z=445 [M+H]⁺

### Example 22

### 6-(Azetidin-1-yl)-4-fluoro-N-(2-iodobenzene-1-sulfonyl)-1-benzofuran-2-carboxamide

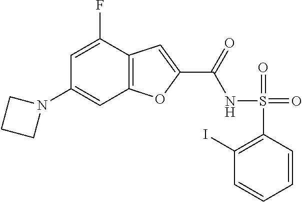 [SMILES: O=C(NS(=O)(=O)c1ccccc1I)c1cc2c(F)cc(N3CCC3)cc2o1]

Synthesized and purified analogously to Example 3 using commercially available 2-iodobenzene-1-sulfonamide (CAS: 53730-99-7, 144 mg, 510 μmol) instead of 2-(cyclopentyloxy)-5-ethylbenzene-1-sulfonamide to give 38.0 mg (17% yield) of the title compound as a yellow solid. ¹H-NMR (400 MHz, DMSO-d₆) δ [ppm]: 2.075 (8.07), 2.290 (1.02), 2.307 (3.19), 2.326 (6.01), 2.336 (1.61), 2.345 (3.49), 2.363 (1.10), 2.518 (4.69), 2.523 (3.16), 2.660 (0.48), 2.664 (1.06), 2.669 (1.52), 2.673 (1.09), 2.678 (0.45), 3.876 (9.77), 3.894 (16.00), 3.913 (9.38), 6.319 (3.49), 6.323 (4.19), 6.352 (15.77), 7.341 (1.67), 7.345 (1.72), 7.361 (3.36), 7.364 (3.35), 7.380 (1.98), 7.383 (1.89), 7.634 (2.30), 7.637 (2.33), 7.655 (3.94), 7.673 (2.20), 7.675 (2.06), 8.042 (2.01), 8.111 (4.46), 8.114 (4.55), 8.131 (4.38), 8.133 (4.21), 8.181 (4.56), 8.185 (4.74), 8.202 (4.30), 8.205 (4.13); LC-MS (method 3): Rₜ=1.24 min; MS (ESIpos): m/z=501 [M+H]⁺

### Example 23

### 6-(Azetidin-1-yl)-N-[2-ethoxy-6-(propan-2-yl)benzene-1-sulfonyl]-4-fluoro-1-benzofuran-2-carboxamide

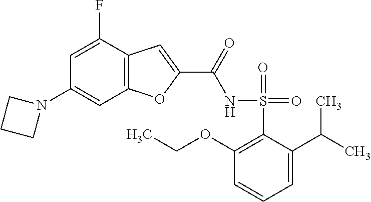 [SMILES: CCOc1cccc(C(C)C)c1S(=O)(=O)NC(=O)c1cc2c(F)cc(N3CCC3)cc2o1]

Synthesized and purified analogously to Example 3 using 2-ethoxy-6-(propan-2-yl)benzene-1-sulfonamide (161 mg, 663 μmol) instead of 2-(cyclopentyloxy)-5-ethylbenzene-1-sulfonamide to give 39.0 mg (85% purity, 13% yield) of the title compound as a brownish solid. ¹H-NMR (400 MHz, DMSO-d₆) δ [ppm]: 1.154 (3.83), 1.161 (1.98), 1.172 (7.58), 1.178 (2.38), 1.183 (6.16), 1.189 (4.50), 1.200 (13.15), 1.218 (7.14), 1.226 (15.81), 1.243 (16.00), 1.352 (0.51), 1.370 (1.00), 1.387 (0.52), 1.987 (12.69), 2.290 (0.52), 2.308 (1.62), 2.326 (2.75), 2.345 (1.90), 2.363 (0.68), 2.522 (3.87), 2.575 (0.44), 2.669 (0.46), 3.876 (4.66), 3.894 (7.94), 3.912 (4.70), 3.999 (0.92), 4.017 (2.73), 4.035 (2.76), 4.053 (0.96), 4.096 (1.36), 4.113 (4.36), 4.130 (4.39), 4.148 (1.47), 4.173 (0.46), 4.191 (0.42), 4.349 (0.99), 4.367 (1.34), 4.383 (1.00), 4.400 (0.41), 6.317 (1.56), 6.322 (2.04), 6.348 (6.82), 6.903 (0.41), 7.013 (1.95), 7.034 (2.16), 7.127 (2.06), 7.145 (2.28), 7.499 (1.37), 7.519 (2.36), 7.540 (1.18), 8.025 (2.02), 12.236 (1.89); LC-MS (method 7): Rₜ=1.40 min; MS (ESIpos): m/z=461 [M+H]⁺

### Example 24

### 6-(Azetidin-1-yl)-N-(2-cyclobutyl-6-fluorobenzene-1-sulfonyl)-4-fluoro-1-benzofuran-2-carboxamide

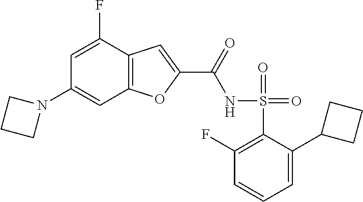 [SMILES: O=C(NS(=O)(=O)c1c(F)cccc1C1CCC1)c1cc2c(F)cc(N3CCC3)cc2o1]

Synthesized and purified analogously to Example 3 using commercially available 2-cyclobutyl-6-fluorobenzene-1-sulfonamide (CAS: 1064286-22-1, 117 mg, 510 μmol) instead of 2-(cyclopentyloxy)-5-ethylbenzene-1-sulfonamide to give 23.0 mg (12% yield) of the title compound as a yellow solid. ¹H-NMR (400 MHz, DMSO-d₆) δ [ppm]: 1.768 (0.45), 1.791 (1.32), 1.817 (1.70), 1.838 (0.77), 1.910 (0.91), 1.934 (2.00), 1.959 (1.88), 1.978 (1.05), 1.983 (1.02), 2.004 (0.44), 2.104 (0.73), 2.109 (0.62), 2.125 (2.42), 2.132 (2.36), 2.149 (3.22), 2.155 (3.60), 2.172 (1.96), 2.178 (2.59), 2.195 (0.52), 2.201 (0.67), 2.281 (1.32), 2.288 (2.53), 2.307 (6.79), 2.315 (3.39), 2.327 (7.12), 2.335 (3.40), 2.343 (4.25), 2.356 (1.21), 2.361 (1.33), 2.518 (1.93), 2.522 (1.25), 2.668 (0.54), 3.877 (9.78), 3.895 (16.00), 3.913 (9.36), 4.472 (0.50), 4.494 (1.51), 4.515 (2.31), 4.537 (1.46), 4.559 (0.46), 6.317 (3.20), 6.321 (5.01), 6.332 (6.06), 6.335 (7.66), 6.338 (6.31), 6.347 (5.21), 6.351 (3.16), 7.243 (1.96), 7.263 (2.28), 7.269 (2.10), 7.271 (2.08), 7.290 (2.08), 7.537 (3.60), 7.556 (4.64), 7.668 (1.72), 7.682 (1.84), 7.689 (2.98), 7.703 (2.86), 7.709 (1.47), 7.722 (1.28), 7.969 (6.36). LC-MS (method 3): Rₜ=1.37 min; MS (ESIneg): m/z=445 [M−H]⁻

### Example 25

### 6-(Azetidin-1-yl)-N-(2-ethoxy-6-methylbenzene-1-sulfonyl)-4-fluoro-1-benzofuran-2-carboxamide

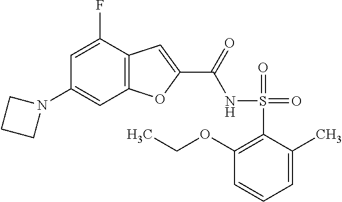 [SMILES: CCOc1cccc(C)c1S(=O)(=O)NC(=O)c1cc2c(F)cc(N3CCC3)cc2o1]

Synthesized and purified analogously to Example 3 using commercially available 2-ethoxy-6-methylbenzene-1-sulfonamide (CAS: 187471-06-3, 150 mg, 697 μmol) instead of 2-(cyclopentyloxy)-5-ethylbenzene-1-sulfonamide to give 85.0 mg (32% yield) of the title compound as an offwhite solid. ¹H-NMR (400 MHz, DMSO-d₆) δ [ppm]: 1.206 (4.99), 1.224 (10.84), 1.241 (5.12), 2.074 (0.81), 2.286 (0.56), 2.304 (1.87), 2.322 (2.88), 2.340 (2.04), 2.359 (0.64), 2.632 (16.00), 3.872 (4.90), 3.890 (8.61), 3.908 (4.77), 4.108 (1.45), 4.126 (4.53), 4.143 (4.46), 4.161 (1.44), 6.314 (1.94), 6.317 (2.07), 6.343 (7.57), 6.930 (2.28), 6.949 (2.48), 7.046 (2.19), 7.067 (2.44), 7.431 (1.69), 7.450 (2.69), 7.471 (1.40), 8.026 (3.21), 12.275 (2.50); LC-MS (method 7): Rₜ=1.29 min; MS (ESIpos): m/z=433 [M+H]⁺

### Example 26

### 6-(Azetidin-1-yl)-N-([1,1′-biphenyl]-2-sulfonyl)-1-benzofuran-2-carboxamide

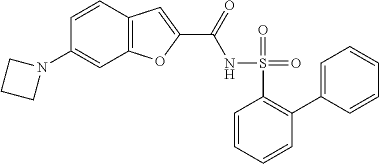 [SMILES: O=C(NS(=O)(=O)c1ccccc1-c1ccccc1)c1cc2ccc(N3CCC3)cc2o1]

Synthesized and purified analogously to Example 3 using 6-(azetidin-1-yl)-1-benzofuran-2-carboxylic acid (Intermediate 5, 210 mg, 967 μmol) and [1,1′-biphenyl]-2-sulfonamide (271 mg, 1.16 mmol) instead of 6-(azetidin-1-yl)-4-fluoro-1-benzofuran-2-carboxylic acid and 2-(cyclopentyloxy)-5-ethylbenzene-1-sulfonamide to give 106 mg (95% purity, 24% yield) of the title compound as a yellow solid. ¹H-NMR (400 MHz, DMSO-d₆) δ [ppm]: 2.077 (3.45), 2.300 (0.96), 2.318 (3.09), 2.325 (1.82), 2.329 (2.12), 2.336 (4.59), 2.355 (3.33), 2.372 (1.07), 2.521 (4.29), 2.525 (2.81), 2.667 (0.73), 2.671 (0.97), 2.676 (0.70), 3.874 (9.36), 3.892 (15.62), 3.910 (9.02), 6.474 (16.00), 6.492 (5.66), 6.497 (3.65), 7.241 (4.32), 7.244 (6.12), 7.250 (1.89), 7.262 (10.05), 7.265 (8.25), 7.293 (3.52), 7.296 (5.63), 7.304 (4.55), 7.308 (5.16), 7.314 (10.52), 7.318 (4.50), 7.322 (5.14), 7.326 (5.02), 7.328 (3.44), 7.332 (5.04), 7.351 (2.53), 7.354 (4.52), 7.358 (2.43), 7.366 (1.52), 7.372 (4.46), 7.379 (0.91), 7.387 (0.82), 7.391 (1.25), 7.394 (0.69), 7.527 (7.50), 7.531 (2.08), 7.550 (6.48), 7.589 (5.91), 7.636 (1.72), 7.640 (1.82), 7.655 (3.80), 7.659 (3.57), 7.675 (3.15), 7.679 (2.85), 7.705 (2.89), 7.709 (3.18), 7.724 (4.08), 7.727 (4.13), 7.743 (1.66), 7.746 (1.55), 8.149 (4.69), 8.152 (5.11), 8.168 (4.21), 8.171 (4.24), 12.024 (0.51); LC-MS (method 1): Rₜ=1.33 min; MS (ESIpos): m/z=433 [M+H]⁺

### Example 27

### 6-(Azetidin-1-yl)-N-(3-chloro-6-methoxy-2-methylbenzene-1-sulfonyl)-4-fluoro-1-benzofuran-2-carboxamide

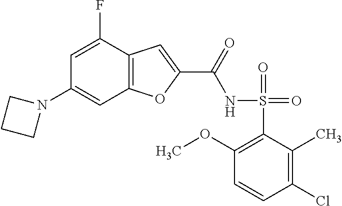 [SMILES: COc1ccc(Cl)c(C)c1S(=O)(=O)NC(=O)c1cc2c(F)cc(N3CCC3)cc2o1]

Synthesized and purified analogously to Example 3 using commercially available 3-chloro-6-methoxy-2-methylbenzene-1-sulfonamide (CAS: 2386443-98-5, 120 mg, 510 μmol) instead of 2-(cyclopentyloxy)-5-ethylbenzene-1-sulfonamide to give 32.0 mg (15% yield) of the title compound as a yellow solid. ¹H-NMR (400 MHz, DMSO-d₆) δ [ppm]: 2.289 (0.45), 2.307 (1.42), 2.326 (2.34), 2.337 (0.88), 2.344 (1.67), 2.363 (0.61), 2.522 (2.73), 2.727 (16.00), 3.355 (2.62), 3.860 (0.54), 3.876 (4.30), 3.894 (7.06), 3.913 (4.27), 6.317 (1.45), 6.321 (2.02), 6.347 (4.65), 7.131 (2.35), 7.153 (2.54), 7.715 (3.23), 7.737 (2.97), 8.030 (3.31), 12.608 (0.40); LC-MS (method 3): Rₜ=1.32 min; MS (ESIpos): m/z=453 [M+H]⁺

### Example 28

### 6-(Azetidin-1-yl)-4-fluoro-N-{2-[(propan-2-yl)oxy]-6-(trifluoromethyl)benzene-1-sulfonyl}-1-benzofuran-2-carboxamide

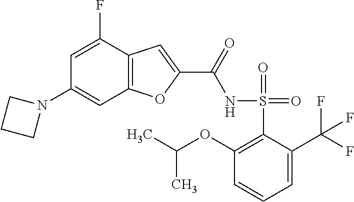 [SMILES: CC(C)Oc1cccc(C(F)(F)F)c1S(=O)(=O)NC(=O)c1cc2c(F)cc(N3CCC3)cc2o1]

Synthesized and purified analogously to Example 3 using 2-[(propan-2-yl)oxy]-6-(trifluoromethyl)benzene-1-sulfonamide (145 mg, 510 μmol) instead of 2-(cyclopentyloxy)-5-ethylbenzene-1-sulfonamide to give 30.0 mg (13% yield) of the title compound as an offwhite solid. ¹H-NMR (400 MHz, DMSO-d₆) δ [ppm]: 1.157 (15.51), 1.173 (16.00), 2.309 (1.26), 2.318 (0.74), 2.327 (2.47), 2.347 (1.52), 2.364 (0.53), 2.518 (2.36), 2.523 (1.50), 2.664 (0.49), 2.669 (0.71), 2.673 (0.50), 3.882 (3.70), 3.900 (6.49), 3.918 (4.30), 3.935 (0.76), 4.868 (0.59), 4.883 (0.78), 4.898 (0.59), 6.322 (2.06), 6.326 (1.35), 6.330 (1.53), 6.354 (4.00), 6.356 (4.15), 6.414 (0.52), 7.527 (1.22), 7.547 (1.42), 7.597 (1.04), 7.618 (1.04), 7.744 (0.74), 7.746 (0.71), 7.752 (1.41), 7.754 (1.27), 7.765 (0.84), 7.770 (0.98), 7.791 (0.43), 8.060 (0.64), 8.075 (0.54), 8.078 (0.71), 8.098 (0.51), 12.386 (0.41); LC-MS (method 7): Rₜ=1.33 min; MS (ESIpos): m/z=501 [M+H]⁺

### Example 29

### 6-(Azetidin-1-yl)-N-{2-chloro-6-[(propan-2-yl)oxy]benzene-1-sulfonyl}-4-fluoro-1-benzofuran-2-carboxamide

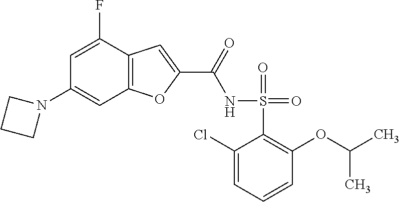 [SMILES: CC(C)Oc1cccc(Cl)c1S(=O)(=O)NC(=O)c1cc2c(F)cc(N3CCC3)cc2o1]

Synthesized and purified analogously to Example 3 using 2-chloro-6-[(propan-2-yl)oxy]benzene-1-sulfonamide (127 mg, 510 μmol) instead of 2-(cyclopentyloxy)-5-ethylbenzene-1-sulfonamide to give 43.0 mg (19% yield) of the title compound as a yellow solid. ¹H-NMR (400 MHz, DMSO-d₆) δ [ppm]: 1.147 (15.55), 1.163 (16.00), 1.186 (0.62), 1.208 (2.29), 1.225 (2.36), 2.309 (1.13), 2.327 (1.77), 2.338 (0.76), 2.346 (1.29), 2.364 (0.46), 2.518 (1.56), 2.523 (1.09), 3.847 (0.59), 3.866 (1.00), 3.881 (3.37), 3.899 (5.43), 3.917 (3.23), 4.785 (0.83), 4.800 (1.14), 4.815 (0.87), 6.323 (1.31), 6.327 (1.50), 6.352 (1.03), 6.356 (1.76), 6.364 (2.48), 6.366 (2.82), 7.167 (1.75), 7.170 (1.99), 7.188 (1.97), 7.189 (2.14), 7.221 (1.50), 7.241 (1.75), 7.507 (1.76), 7.528 (2.20), 7.548 (1.30), 8.038 (2.50), 12.462 (0.40); LC-MS (method 3): Rₜ=1.30 min; MS (ESIneg): m/z=465 [M−H]⁻

### Example 30

### 6-(Azetidin-1-yl)-N-[2,5-di(propan-2-yl)benzene-1-sulfonyl]-4-fluoro-1-benzofuran-2-carboxamide

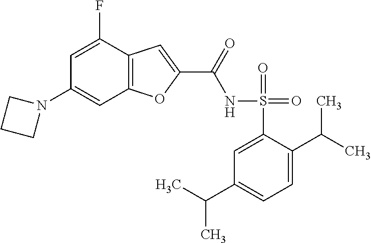 [SMILES: CC(C)c1ccc(C(C)C)c(S(=O)(=O)NC(=O)c2cc3c(F)cc(N4CCC4)cc3o2)c1]

Synthesized and purified analogously to Example 3 using 2,5-di(propan-2-yl)benzene-1-sulfonamide (123 mg, 510 μmol) instead of 2-(cyclopentyloxy)-5-ethylbenzene-1-sulfonamide to give the title compound as a light yellow solid. ¹H-NMR (400 MHz, DMSO-d₆) δ [ppm]: 1.128 (9.62), 1.145 (9.54), 1.220 (15.97), 1.237 (16.00), 2.074 (0.88), 2.304 (1.09), 2.322 (1.97), 2.331 (0.87), 2.341 (1.19), 2.518 (1.98), 2.523 (1.29), 2.664 (0.46), 2.669 (0.63), 2.673 (0.45), 2.964 (0.83), 2.981 (1.09), 2.998 (0.78), 3.870 (3.40), 3.889 (5.63), 3.907 (3.35), 6.313 (1.10), 6.317 (1.39), 6.339 (3.20), 6.341 (3.26), 7.513 (0.53), 7.533 (2.51), 7.541 (1.74), 7.854 (2.46), 7.859 (2.29), 7.938 (0.53); LC-MS (method 7): Rₜ=1.49 min; MS (ESIpos): m/z=459 [M+H]⁺

### Example 31

### 6-(Azetidin-1-yl)-4-fluoro-N-(2-propoxy-6-propylbenzene-1-sulfonyl)-1-benzofuran-2-carboxamide

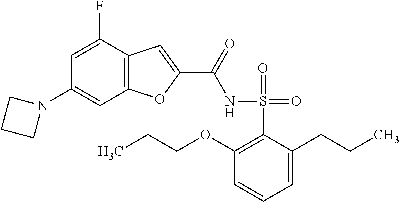 [SMILES: CCCOc1cccc(CCC)c1S(=O)(=O)NC(=O)c1cc2c(F)cc(N3CCC3)cc2o1]

Synthesized and purified analogously to Example 3 using 2-propoxy-6-propylbenzene-1-sulfonamide (150 mg, 583 μmol) instead of 2-(cyclopentyloxy)-5-ethylbenzene-1-sulfonamide to give 70.0 mg (29% yield) of the title compound as a light green solid. ¹H-NMR (400 MHz, DMSO-d₆) δ [ppm]: 0.831 (6.96), 0.850 (16.00), 0.868 (7.42), 0.915 (5.18), 0.933 (12.41), 0.952 (5.56), 1.624 (1.38), 1.643 (2.24), 1.650 (1.61), 1.662 (2.45), 1.668 (3.03), 1.686 (4.43), 1.704 (3.86), 1.721 (1.84), 1.739 (0.42), 2.074 (0.91), 2.287 (0.61), 2.305 (1.93), 2.323 (2.94), 2.342 (2.15), 2.360 (0.68), 2.518 (1.33), 2.523 (0.84), 3.029 (2.40), 3.044 (2.13), 3.049 (2.63), 3.053 (2.22), 3.069 (2.25), 3.872 (5.67), 3.891 (9.49), 3.909 (5.44), 3.993 (2.59), 4.010 (5.22), 4.026 (2.51), 6.311 (1.79), 6.314 (2.40), 6.332 (4.99), 6.340 (2.86), 6.924 (2.27), 6.943 (2.40), 7.032 (1.95), 7.053 (2.13), 7.442 (1.36), 7.461 (2.19), 7.482 (1.13), 8.007 (2.00), 12.136 (2.47); LC-MS (method 7): Rₜ=1.48 min; MS (ESIpos): m/z=475 [M+H]⁺

### Example 32

### 6-(Azetidin-1-yl)-4-fluoro-N-[2-methyl-5-(propan-2-yl)benzene-1-sulfonyl]-1-benzofuran-2-carboxamide

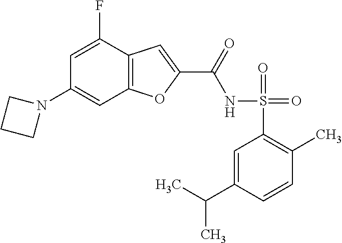 [SMILES: Cc1ccc(C(C)C)cc1S(=O)(=O)NC(=O)c1cc2c(F)cc(N3CCC3)cc2o1]

Synthesized and purified analogously to Example 3 using commercially available 2-methyl-5-(propan-2-yl)benzene-1-sulfonamide (CAS: 1094713-96-8, 109 mg, 510 μmol) instead of 2-(cyclopentyloxy)-5-ethylbenzene-1-sulfonamide to give 30.0 mg (95% purity, 16% yield) of the title compound as a yellow solid. ¹H-NMR (400 MHz, DMSO-d₆) δ [ppm]: 1.211 (16.00), 1.229 (15.70), 2.074 (1.75), 2.305 (1.03), 2.323 (1.62), 2.342 (1.16), 2.518 (1.09), 2.522 (0.72), 2.568 (10.29), 2.966 (0.77), 2.983 (1.02), 3.000 (0.73), 3.873 (3.12), 3.891 (5.17), 3.909 (2.99), 6.312 (1.09), 6.316 (1.42), 6.340 (3.10), 7.314 (1.44), 7.335 (1.87), 7.468 (1.24), 7.473 (1.27), 7.488 (0.96), 7.492 (0.99), 7.879 (2.39), 7.883 (2.31), 7.955 (1.93); LC-MS (method 3): Rₜ=1.39 min; MS (ESIneg): m/z=429 [M−H]⁻

### Example 33

### 6-(Azetidin-1-yl)-N-[2-(cyclopropylmethoxy)-6-ethylbenzene-1-sulfonyl]-4-fluoro-1-benzofuran-2-carboxamide

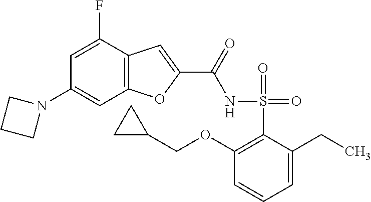 [SMILES: CCc1cccc(OCC2CC2)c1S(=O)(=O)NC(=O)c1cc2c(F)cc(N3CCC3)cc2o1]

Synthesized and purified analogously to Example 3 using 2-(cyclopropylmethoxy)-6-ethylbenzene-1-sulfonamide (150 mg, 587 μmol) instead of 2-(cyclopentyloxy)-5-ethylbenzene-1-sulfonamide to give 36.0 mg (15% yield) of the title compound as an ochre solid. ¹H-NMR (400 MHz, DMSO-d₆) δ [ppm]: 0.164 (4.27), 0.285 (2.03), 0.295 (5.17), 0.300 (5.39), 0.305 (3.24), 0.315 (5.84), 0.319 (5.60), 0.331 (2.06), 1.216 (7.58), 1.234 (14.90), 1.253 (7.35), 2.074 (7.19), 2.289 (1.18), 2.308 (3.59), 2.326 (6.61), 2.344 (4.32), 2.362 (1.56), 2.436 (0.43), 2.453 (0.48), 2.522 (15.43), 2.539 (9.40), 2.660 (0.54), 2.665 (1.13), 2.669 (1.59), 2.673 (1.21), 3.085 (2.34), 3.103 (7.15), 3.122 (7.11), 3.140 (2.43), 3.875 (9.03), 3.894 (16.00), 3.912 (11.90), 3.934 (6.34), 6.315 (2.73), 6.340 (11.68), 6.944 (2.38), 6.963 (2.76), 7.044 (1.79), 7.064 (2.07), 7.450 (1.18), 7.471 (1.92), 7.490 (1.18), 8.019 (2.19), 12.160 (3.30); LC-MS (method 7): Rₜ=1.42 min; MS (ESIpos): m/z=473 [M+H]⁺

### Example 34

### 6-(Azetidin-1-yl)-N-(5-ethyl-2-methylbenzene-1-sulfonyl)-4-fluoro-1-benzofuran-2-carboxamide

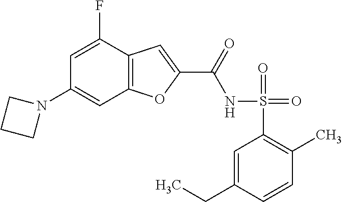 [SMILES: CCc1ccc(C)c(S(=O)(=O)NC(=O)c2cc3c(F)cc(N4CCC4)cc3o2)c1]

Synthesize and purified analogously to Example 3 using commercially available 5-ethyl-2-methylbenzene-1-sulfonamide (CAS: 1094671-90-5, 102 mg, 510 μmol) instead of 2-(cyclopentyloxy)-5-ethylbenzene-1-sulfonamide to give 35.0 mg (18% yield) of the title compound as light green solid. ¹H-NMR (400 MHz, DMSO-d₆) δ [ppm]: 1.139 (0.91), 1.177 (6.56), 1.196 (16.00), 1.215 (6.60), 2.288 (0.52), 2.306 (1.64), 2.326 (2.79), 2.337 (0.87), 2.344 (1.86), 2.361 (0.65), 2.373 (1.09), 2.518 (2.17), 2.523 (1.62), 2.567 (14.07), 2.651 (1.13), 2.670 (3.85), 2.689 (3.31), 2.708 (1.04), 3.874 (5.07), 3.893 (8.25), 3.911 (4.86), 6.313 (1.83), 6.318 (2.32), 6.322 (0.80), 6.344 (6.55), 7.304 (1.91), 7.324 (2.58), 7.427 (1.63), 7.431 (1.65), 7.447 (1.21), 7.451 (1.22), 7.854 (3.07), 7.859 (2.96), 7.948 (1.82); LC-MS (method 7): Rₜ=1.34 min; MS (ESIpos): m/z=417 [M+H]⁺

### Example 35

### 6-(Azetidin-1-yl)-N-[2-(difluoromethoxy)-4-methylbenzene-1-sulfonyl]-4-fluoro-1-benzofuran-2-carboxamide

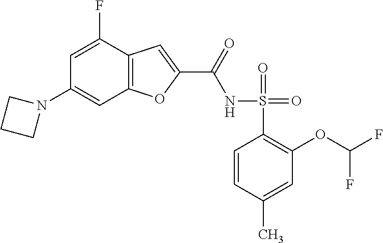 [SMILES: Cc1ccc(S(=O)(=O)NC(=O)c2cc3c(F)cc(N4CCC4)cc3o2)c(OC(F)F)c1]

Synthesized and purified analogously to Example 3 using commercially available 2-(difluoromethoxy)-4-methylbenzene-1-sulfonamide (CAS: 187471-19-8, 121 mg, 510 μmol) instead of 2-(cyclopentyloxy)-5-ethylbenzene-1-sulfonamide to give 106 mg (48% yield, could contain ammonium salt) of the title compound as a yellow solid. ¹H-NMR (400 MHz, DMSO-d₆) δ [ppm]: 1.209 (0.68), 1.225 (0.70), 2.285 (0.56), 2.302 (1.72), 2.321 (2.56), 2.332 (0.78), 2.340 (1.87), 2.358 (0.61), 2.408 (16.00), 3.870 (5.12), 3.878 (1.23), 3.889 (8.38), 3.899 (1.15), 3.906 (4.88), 6.311 (1.98), 6.315 (2.36), 6.344 (8.93), 6.349 (1.95), 7.152 (2.34), 7.242 (3.54), 7.268 (1.74), 7.270 (2.08), 7.272 (1.86), 7.274 (1.44), 7.290 (2.17), 7.292 (1.98), 7.294 (1.67), 7.334 (4.78), 7.515 (2.15), 7.922 (4.80), 7.943 (4.36), 7.989 (4.28); LC-MS (method 3): Rₜ=1.26 min; MS (ESIpos): m/z=455 [M+H]⁺

### Example 36

### 6-(Azetidin-1-yl)-N-(4-ethyl-2-methylthiophene-3-sulfonyl)-4-fluoro-1-benzofuran-2-carboxamide

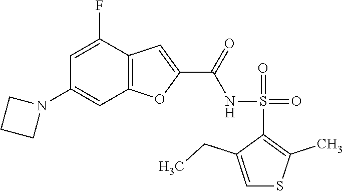 [SMILES: CCc1csc(C)c1S(=O)(=O)NC(=O)c1cc2c(F)cc(N3CCC3)cc2o1]

Synthesized and purified analogously to Example 3 using 4-ethyl-2-methylthiophene-3-sulfonamide (105 mg, 510 μmol) instead of 2-(cyclopentyloxy)-5-ethylbenzene-1-sulfonamide to give 60.0 mg (32% yield) as a yellow solid. ¹H-NMR (400 MHz, DMSO-d₆) δ [ppm]: 1.161 (3.91), 1.180 (8.42), 1.198 (4.03), 2.074 (0.98), 2.306 (1.23), 2.325 (1.84), 2.344 (1.34), 2.361 (0.43), 2.727 (16.00), 2.774 (0.79), 2.790 (2.36), 2.793 (2.38), 2.808 (2.28), 2.811 (2.32), 2.827 (0.72), 3.372 (0.61), 3.875 (3.60), 3.894 (6.02), 3.912 (3.44), 6.308 (1.23), 6.312 (1.68), 6.330 (3.04), 6.338 (2.03), 7.093 (3.78), 7.947 (3.37); LC-MS (method 3): Rₜ=1.35 min; MS (ESIpos): m/z=423 [M+H]⁺

### Example 37

### 6-(Azetidin-1-yl)-4-fluoro-N-[2-(3-methyl-1,2,4-oxadiazol-5-yl)benzene-1-sulfonyl]-1-benzofuran-2-carboxamide

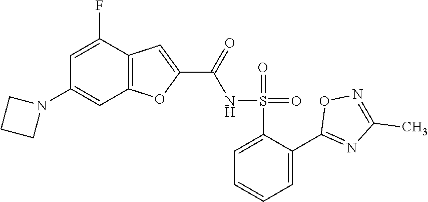 [SMILES: Cc1noc(-c2ccccc2S(=O)(=O)NC(=O)c2cc3c(F)cc(N4CCC4)cc3o2)n1]

Synthesized and purified analogously to Example 3 using commercially available 2-(3-methyl-1,2,4-oxadiazol-5-yl)benzene-1-sulfonamide (CAS: 92737-51-4, 122 mg, 510 μmol) instead of 2-(cyclopentyloxy)-5-ethylbenzene-1-sulfonamide to give 27.0 mg (13% yield) of the title compound as a yellow solid. ¹H-NMR (400 MHz, DMSO-d₆) δ [ppm]: 2.309 (0.95), 2.327 (1.55), 2.347 (1.04), 2.427 (16.00), 2.518 (0.83), 2.522 (0.58), 3.880 (2.94), 3.899 (4.81), 3.917 (2.82), 6.323 (1.09), 6.327 (1.34), 6.356 (5.33), 7.832 (0.69), 7.836 (0.87), 7.851 (1.18), 7.855 (1.45), 7.879 (0.55), 7.882 (0.65), 7.897 (1.32), 7.901 (1.28), 7.917 (1.79), 7.922 (1.87), 7.936 (1.56), 7.940 (1.41), 7.955 (0.56), 7.959 (0.44), 8.285 (1.37), 8.288 (1.14), 8.304 (1.39), 8.308 (1.16); LC-MS (method 3): Rₜ=1.21 min; MS (ESIpos): m/z=457 [M+H]⁺

### Example 38

### 6-(Azetidin-1-yl)-N-[4-bromo-2-(propan-2-yl)benzene-1-sulfonyl]-4-fluoro-1-benzofuran-2-carboxamide

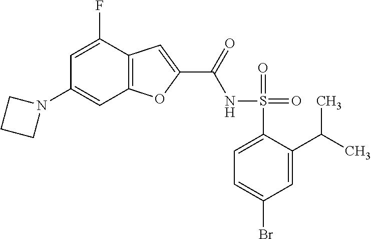 [SMILES: CC(C)c1cc(Br)ccc1S(=O)(=O)NC(=O)c1cc2c(F)cc(N3CCC3)cc2o1]

Synthesized and purified analogously to Example 3 using 4-bromo-2-(propan-2-yl)benzene-1-sulfonamide (142 mg, 510 μmol) instead of 2-(cyclopentyloxy)-5-ethylbenzene-1-sulfonamide to give 53.0 mg (24% yield) of the title compound as a green solid. ¹H-NMR (400 MHz, DMSO-d₆) δ [ppm]: 1.149 (15.19), 1.165 (16.00), 2.285 (0.53), 2.303 (1.68), 2.322 (2.63), 2.331 (1.02), 2.340 (2.00), 2.358 (0.71), 2.522 (1.98), 2.539 (0.75), 3.849 (0.50), 3.871 (5.38), 3.890 (8.88), 3.899 (2.86), 3.908 (5.41), 6.313 (1.84), 6.317 (2.39), 6.345 (9.16), 7.650 (2.37), 7.655 (2.60), 7.671 (2.69), 7.676 (3.02), 7.816 (4.08), 7.821 (4.09), 7.927 (6.23), 7.942 (3.73), 7.949 (5.95); LC-MS (method 3): Rₜ=1.42 min; MS (ESIneg): m/z=493 [M−H]⁻

### Example 39

### 6-(Azetidin-1-yl)-N-{2-bromo-6-[(propan-2-yl)oxy]benzene-1-sulfonyl}-4-fluoro-1-benzofuran-2-carboxamide

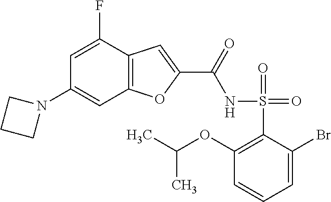 [SMILES: CC(C)Oc1cccc(Br)c1S(=O)(=O)NC(=O)c1cc2c(F)cc(N3CCC3)cc2o1]

Synthesized and purified analogously to Example 3 using 2-bromo-6-[(propan-2-yl)oxy]benzene-1-sulfonamide (150 mg, 510 μmol) instead of 2-(cyclopentyloxy)-5-ethylbenzene-1-sulfonamide to give 63.0 mg (26% yield) of the title compound as a yellow solid. ¹H-NMR (400 MHz, DMSO-d₆) δ [ppm]: 1.141 (15.53), 1.156 (16.00), 1.207 (1.09), 1.224 (1.06), 2.306 (1.16), 2.324 (1.73), 2.343 (1.34), 2.361 (0.47), 2.518 (0.82), 2.523 (0.56), 3.365 (0.68), 3.863 (0.54), 3.878 (3.44), 3.896 (5.72), 3.915 (3.41), 4.780 (0.88), 4.795 (1.21), 4.811 (0.92), 6.318 (1.34), 6.322 (1.68), 6.347 (1.16), 6.351 (1.80), 6.362 (2.96), 7.251 (1.04), 7.255 (1.22), 7.272 (1.52), 7.275 (1.55), 7.377 (0.74), 7.381 (1.22), 7.397 (3.06), 7.401 (2.59), 7.410 (2.99), 7.430 (2.72), 7.450 (0.91), 8.042 (3.36), 12.414 (0.46); LC-MS (method 3): Rₜ=1.31 min; MS (ESIpos): m/z=509 [M+H]⁺

### Example 40

### 6-(Azetidin-1-yl)-4-fluoro-N-(3-propylbenzene-1-sulfonyl)-1-benzofuran-2-carboxamide

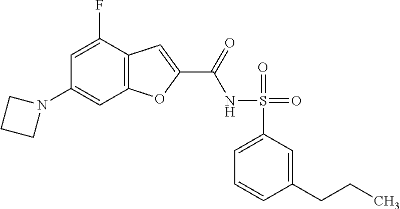 [SMILES: CCCc1cccc(S(=O)(=O)NC(=O)c2cc3c(F)cc(N4CCC4)cc3o2)c1]

Synthesized and purified analogously to Example 3 using commercially available 3-propylbenzene-1-sulfonamide (CAS: 196107-64-9, 132 mg, 663 μmol) instead of 2-(cyclopentyloxy)-5-ethylbenzene-1-sulfonamide to give 135 mg (95% purity, 56% yield) of the title compound as a yellow solid. ¹H-NMR (400 MHz, DMSO-d₆) δ [ppm]: 0.870 (7.11), 0.889 (16.00), 0.907 (7.38), 1.558 (0.51), 1.575 (2.02), 1.594 (3.28), 1.613 (3.19), 1.632 (1.91), 1.650 (0.45), 1.732 (0.92), 2.286 (0.60), 2.304 (1.85), 2.323 (2.82), 2.332 (0.85), 2.342 (2.00), 2.359 (0.65), 2.518 (0.87), 2.523 (0.62), 2.646 (3.02), 2.665 (4.03), 2.684 (2.80), 3.067 (0.40), 3.074 (0.76), 3.084 (0.73), 3.374 (0.61), 3.872 (5.75), 3.890 (9.32), 3.908 (5.43), 6.305 (2.22), 6.309 (2.52), 6.334 (1.68), 6.339 (3.35), 6.344 (4.40), 6.345 (4.82), 6.349 (2.72), 7.524 (0.43), 7.544 (3.28), 7.546 (4.62), 7.554 (4.67), 7.559 (7.05), 7.566 (0.55), 7.570 (0.46), 7.791 (0.52), 7.799 (5.88), 7.807 (5.19), 7.815 (0.76), 7.818 (1.55), 7.823 (0.92), 7.880 (5.77), 7.882 (5.78); LC-MS (method 7): Rₜ=1.36 min; MS (ESIpos): m/z=417 [M+H]⁺

### Example 41

### 6-(Azetidin-1-yl)-4-fluoro-N-(1-methyl-1H-indole-7-sulfonyl)-1-benzofuran-2-carboxamide

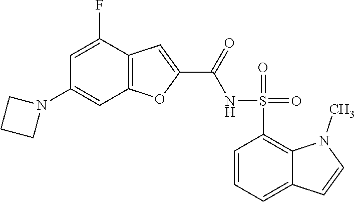 [SMILES: Cn1ccc2cccc(S(=O)(=O)NC(=O)c3cc4c(F)cc(N5CCC5)cc4o3)c21]

Synthesized and purified analogously to Example 3 using commercially available 1-methyl-1H-indole-7-sulfonamide (CAS: 2168249-75-8, 107 mg, 510 μmol) instead of 2-(cyclopentyloxy)-5-ethylbenzene-1-sulfonamide to give 30.0 mg (16% yield) of the title compound as a light yellow solid. ¹H-NMR (400 MHz, DMSO-d₆) δ [ppm]: 2.074 (0.57), 2.302 (1.22), 2.321 (1.94), 2.332 (0.72), 2.340 (1.35), 2.358 (0.42), 2.518 (1.24), 2.523 (0.80), 3.870 (3.70), 3.888 (6.04), 3.906 (3.47), 4.181 (16.00), 6.311 (1.31), 6.314 (1.57), 6.344 (2.68), 6.346 (3.38), 6.348 (3.35), 6.653 (3.43), 6.661 (3.56), 7.188 (1.62), 7.207 (3.20), 7.226 (1.63), 7.435 (2.78), 7.442 (2.65), 7.889 (1.73), 7.891 (2.16), 7.908 (2.02), 7.912 (2.93), 7.932 (2.82); LC-MS (method 7): Rₜ=1.25 min; MS (ESIpos): m/z=428 [M+H]⁺

### Example 42

### 6-(Azetidin-1-yl)-4-fluoro-N-{2-fluoro-6-[(propan-2-yl)amino]benzene-1-sulfonyl}-1-benzofuran-2-carboxamide

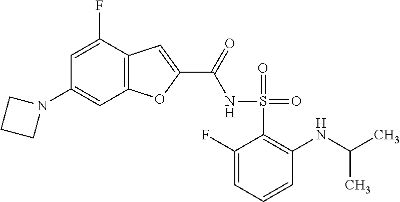 [SMILES: CC(C)Nc1cccc(F)c1S(=O)(=O)NC(=O)c1cc2c(F)cc(N3CCC3)cc2o1]

Synthesized and purified analogously to Example 3 using commercially available 2-fluoro-6-[(propan-2-yl)amino]benzene-1-sulfonamide (CAS: 1821384-82-0, 75.0 mg, 323 μmol) instead of 2-(cyclopentyloxy)-5-ethylbenzene-1-sulfonamide to give 50.0 mg (39% yield) of the title compound as a yellow solid. ¹H-NMR (400 MHz, DMSO-d₆) δ [ppm]: 1.208 (15.62), 1.224 (16.00), 2.309 (1.14), 2.318 (0.53), 2.327 (1.90), 2.346 (1.26), 2.364 (0.40), 2.518 (1.34), 2.522 (0.83), 3.380 (0.57), 3.751 (0.74), 3.782 (0.73), 3.879 (3.49), 3.898 (5.76), 3.916 (3.32), 6.316 (1.27), 6.319 (1.64), 6.346 (6.14), 6.382 (0.85), 6.403 (0.93), 6.411 (0.90), 6.430 (0.85), 6.649 (1.47), 6.672 (1.58), 7.345 (0.57), 7.361 (0.70), 7.365 (0.98), 7.381 (0.95), 7.386 (0.71), 7.403 (0.55), 7.948 (2.11); LC-MS (method 7): Rₜ=1.38 min; MS (ESIpos): m/z=450 [M+H]⁺

### Example 43

### 6-(Azetidin-1-yl)-4-fluoro-N-(2-fluoro-5-methylbenzene-1-sulfonyl)-1-benzofuran-2-carboxamide

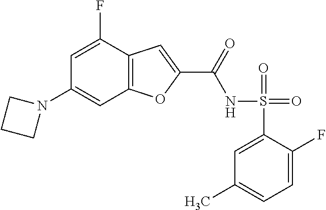 [SMILES: Cc1ccc(F)c(S(=O)(=O)NC(=O)c2cc3c(F)cc(N4CCC4)cc3o2)c1]

Synthesized and purified analogously to Example 3 using commercially available 2-fluoro-5-methylbenzene-1-sulfonamide (CAS: 438539-71-0, 96.5 mg, 510 μmol) instead of 2-(cyclopentyloxy)-5-ethylbenzene-1-sulfonamide to give 32.0 mg (18% yield) of the title compound as a yellow solid. ¹H-NMR (400 MHz, DMSO-d₆) δ [ppm]: 2.074 (2.37), 2.289 (0.67), 2.308 (2.09), 2.326 (3.42), 2.345 (2.47), 2.363 (0.92), 2.392 (16.00), 2.518 (4.64), 2.522 (3.30), 2.669 (0.42), 3.878 (6.38), 3.896 (10.63), 3.915 (6.48), 6.316 (2.21), 6.320 (2.97), 6.346 (9.92), 7.320 (1.76), 7.341 (2.31), 7.346 (2.10), 7.367 (2.14), 7.554 (0.89), 7.559 (1.09), 7.565 (1.14), 7.572 (1.33), 7.580 (1.08), 7.586 (0.98), 7.591 (0.92), 7.774 (1.68), 7.779 (1.76), 7.792 (1.83), 7.796 (1.76), 7.964 (4.35); LC-MS (method 3): Rₜ=1.22 min; MS (ESIpos): m/z=407 [M+H]⁺

### Example 44

### 6-(Azetidin-1-yl)-N-[2-(3-ethyl-1,2,4-oxadiazol-5-yl)benzene-1-sulfonyl]-4-fluoro-1-benzofuran-2-carboxamide

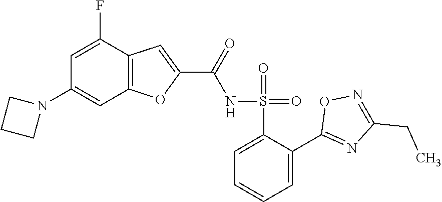 [SMILES: CCc1noc(-c2ccccc2S(=O)(=O)NC(=O)c2cc3c(F)cc(N4CCC4)cc3o2)n1]

Synthesized and purified analogously to Example 3 using 2-(3-ethyl-1,2,4-oxadiazol-5-yl)benzene-1-sulfonamide (129 mg, 510 μmol) instead of 2-(cyclopentyloxy)-5-ethylbenzene-1-sulfonamide to give 59.0 mg (27% yield) of the title compound as a yellow solid. ¹H-NMR (400 MHz, DMSO-d₆) δ [ppm]: 1.209 (0.86), 1.214 (7.24), 1.226 (1.04), 1.232 (16.00), 1.251 (7.62), 2.075 (0.63), 2.288 (0.51), 2.307 (1.57), 2.326 (2.38), 2.344 (1.76), 2.362 (0.59), 2.518 (0.84), 2.523 (0.56), 2.788 (2.04), 2.807 (6.49), 2.826 (6.27), 2.845 (1.85), 3.866 (0.43), 3.878 (4.79), 3.896 (7.99), 3.915 (4.83), 3.930 (0.48), 6.319 (1.81), 6.323 (3.13), 6.349 (6.92), 7.749 (0.43), 7.751 (0.44), 7.844 (1.14), 7.848 (1.57), 7.861 (1.97), 7.863 (2.17), 7.866 (2.70), 7.884 (1.05), 7.887 (1.34), 7.902 (2.37), 7.906 (2.25), 7.920 (1.79), 7.923 (2.68), 7.927 (2.43), 7.936 (2.94), 7.942 (2.92), 7.946 (2.53), 7.960 (0.95), 7.965 (0.83), 8.293 (2.26), 8.295 (2.02), 8.297 (1.80), 8.311 (2.35), 8.315 (1.99); LC-MS (method 3): Rₜ=1.30 min; MS (ESIpos): m/z=471 [M+H]⁺

### Example 45

### 6-(Azetidin-1-yl)-N-[2-(benzenesulfonyl)benzene-1-sulfonyl]-4-fluoro-1-benzofuran-2-carboxamide

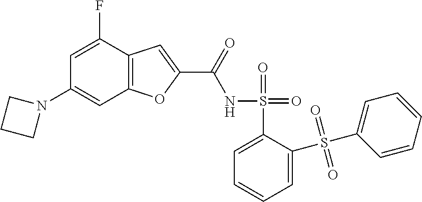 [SMILES: O=C(NS(=O)(=O)c1ccccc1S(=O)(=O)c1ccccc1)c1cc2c(F)cc(N3CCC3)cc2o1]

Synthesized analogously to Example 3 using 2-(benzenesulfonyl)benzene-1-sulfonamide (152 mg, 510 μmol) instead of 2-(cyclopentyloxy)-5-ethylbenzene-1-sulfonamide and purified by HT-HPLC basic method to give 3.00 mg (1% yield, could contain ammonia salt) of the title compound as a yellow solid. ¹H-NMR (400 MHz, DMSO-d₆) δ [ppm]: 2.289 (0.82), 2.307 (2.63), 2.327 (5.31), 2.344 (3.22), 2.362 (1.15), 2.522 (16.00), 2.539 (9.77), 2.665 (1.26), 2.669 (1.78), 2.673 (1.36), 3.871 (6.58), 3.889 (11.61), 3.907 (6.94), 6.295 (1.76), 6.324 (1.84), 6.354 (6.38), 6.952 (4.02), 7.080 (4.47), 7.207 (3.95), 7.509 (1.54), 7.527 (3.68), 7.545 (2.86), 7.597 (1.52), 7.615 (1.89), 7.924 (6.81), 7.942 (6.10), 8.305 (1.49), 8.316 (1.75), 8.327 (1.59), 8.375 (1.69), 8.385 (1.97), 8.397 (1.77); LC-MS (method 7): Rₜ=1.30 min; MS (ESIpos): m/z=515 [M+H]⁺

### Example 46

### 6-(azetidin-1-yl)-N-[2-(ethylamino)benzene-1-sulfonyl]-4-fluoro-1-benzofuran-2-carboxamide

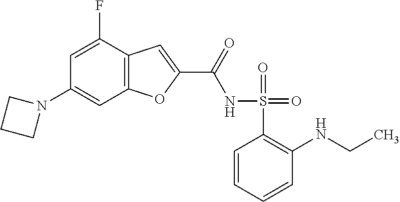 [SMILES: CCNc1ccccc1S(=O)(=O)NC(=O)c1cc2c(F)cc(N3CCC3)cc2o1]

Synthesized and purified analogously to Example 3 using 2-(ethylamino)benzene-1-sulfonamide (75.0 mg, 375 μmol) instead of 2-(cyclopentyloxy)-5-ethylbenzene-1-sulfonamide to give 70.0 mg (51% yield) of the title compound as an offwhite solid. ¹H-NMR (400 MHz, DMSO-d₆) δ [ppm]: 1.170 (6.90), 1.188 (16.00), 1.206 (7.28), 2.074 (3.63), 2.290 (0.60), 2.308 (1.87), 2.326 (3.19), 2.345 (2.23), 2.363 (0.79), 2.518 (5.65), 2.522 (4.05), 2.539 (2.22), 2.669 (0.47), 3.188 (1.70), 3.206 (5.32), 3.224 (5.38), 3.242 (1.88), 3.877 (5.78), 3.895 (9.66), 3.913 (5.89), 6.314 (2.03), 6.317 (2.69), 6.345 (10.47), 6.679 (1.39), 6.681 (1.64), 6.699 (3.01), 6.701 (2.24), 6.717 (1.65), 6.719 (1.74), 6.792 (2.63), 6.813 (2.82), 7.411 (1.37), 7.415 (1.63), 7.429 (1.57), 7.433 (2.56), 7.436 (1.72), 7.450 (1.41), 7.454 (1.39), 7.724 (2.84), 7.728 (3.09), 7.743 (2.80), 7.748 (2.86), 7.844 (5.19); LC-MS (method 7): Rₜ=1.29 min; MS (ESIpos): m/z=418 [M+H]⁺

### Example 47

### 6-(Azetidin-1-yl)-N-{2-[ethyl(methyl)carbamoyl]benzene-1-sulfonyl}-4-fluoro-1-benzofuran-2-carboxamide

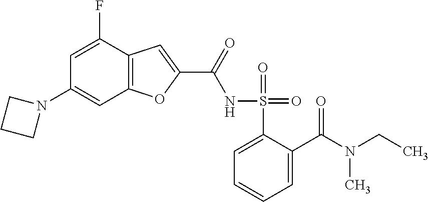 [SMILES: CCN(C)C(=O)c1ccccc1S(=O)(=O)NC(=O)c1cc2c(F)cc(N3CCC3)cc2o1]

Synthesized and purified analogously to Example 3 using N-ethyl-N-methyl-2-sulfamoylbenzamide (124 mg, 510 μmol) instead of 2-(cyclopentyloxy)-5-ethylbenzene-1-sulfonamide to give 11.0 mg (5% yield) of the title compound as a yellow solid. ¹H-NMR (500 MHz, DMSO-d₆) δ [ppm]: 1.035 (3.28), 1.049 (7.30), 1.063 (3.38), 1.161 (3.63), 1.176 (8.05), 1.190 (3.70), 2.327 (1.17), 2.342 (3.26), 2.357 (5.60), 2.365 (1.67), 2.371 (3.63), 2.386 (1.26), 2.513 (4.82), 2.517 (3.84), 2.521 (3.09), 2.627 (0.50), 2.631 (0.98), 2.635 (1.42), 2.638 (1.02), 2.642 (0.52), 2.754 (15.37), 2.983 (15.87), 3.120 (1.56), 3.494 (1.18), 3.508 (1.15), 3.910 (10.31), 3.924 (16.00), 3.939 (10.02), 6.272 (2.67), 6.276 (2.96), 6.296 (2.52), 6.299 (3.14), 6.320 (5.69), 7.355 (1.44), 7.370 (1.59), 7.390 (1.35), 7.403 (1.46), 7.617 (0.71), 7.633 (1.58), 7.640 (1.53), 7.646 (1.23), 7.649 (1.14), 7.656 (1.08), 7.708 (2.05), 7.720 (3.15), 7.722 (3.17), 7.736 (1.38), 7.850 (2.78), 7.868 (2.76), 8.091 (1.70), 8.107 (3.05), 8.123 (1.48); LC-MS (method 3): Rₜ=1.18 min; MS (ESIpos): m/z=460 [M+H]⁺

### Example 48

### 6-(Azetidin-1-yl)-4-fluoro-N-[3-(methoxymethyl)benzene-1-sulfonyl]-1-benzofuran-2-carboxamide

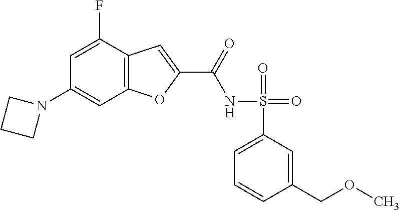 [SMILES: COCc1cccc(S(=O)(=O)NC(=O)c2cc3c(F)cc(N4CCC4)cc3o2)c1]

Synthesized and purified analogously to Example 3 using 3-(methoxymethyl)benzene-1-sulfonamide (133 mg, 663 μmol) instead of 2-(cyclopentyloxy)-5-ethylbenzene-1-sulfonamide to give 120 mg (49% yield) of the title compound as a yellow solid. ¹H-NMR (400 MHz, DMSO-d₆) δ [ppm]: 2.075 (4.02), 2.288 (0.75), 2.306 (2.30), 2.325 (3.66), 2.336 (1.05), 2.344 (2.52), 2.362 (0.81), 2.518 (2.21), 2.523 (1.50), 2.539 (0.49), 2.665 (0.47), 2.669 (0.68), 2.673 (0.48), 3.291 (0.60), 3.294 (0.71), 3.376 (1.56), 3.874 (7.25), 3.893 (11.72), 3.911 (6.90), 4.525 (16.00), 6.309 (2.74), 6.313 (3.25), 6.338 (1.91), 6.344 (6.92), 6.346 (6.45), 7.601 (0.96), 7.619 (3.69), 7.637 (8.19), 7.641 (3.55), 7.651 (0.68), 7.655 (0.69), 7.872 (5.40), 7.895 (1.53), 7.901 (2.90), 7.905 (1.97), 7.912 (1.15), 7.917 (2.47), 7.922 (1.63), 7.938 (3.54), 7.941 (4.44); LC-MS (method 7): Rₜ=1.16 min; MS (ESIpos): m/z=419 [M+H]⁺

### Example 49

### 6-(Azetidin-1-yl)-N-{5-chloro-2-[(propan-2-yl)amino]benzene-1-sulfonyl}-4-fluoro-1-benzofuran-2-carboxamide

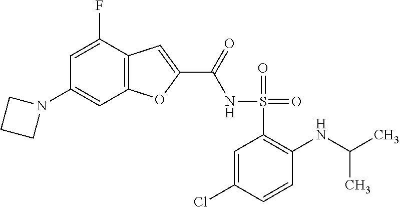 [SMILES: CC(C)Nc1ccc(Cl)cc1S(=O)(=O)NC(=O)c1cc2c(F)cc(N3CCC3)cc2o1]

Synthesized and purified analogously to Example 3 using 5-chloro-2-[(propan-2-yl)amino]benzene-1-sulfonamide (75.0 mg, 302 μmol) instead of 2-(cyclopentyloxy)-5-ethylbenzene-1-sulfonamide to give 65.0 mg (53% yield) of the title compound as a yellow solid. ¹H-NMR (400 MHz, DMSO-d₆) δ [ppm]: 1.147 (16.00), 1.163 (15.71), 2.074 (8.86), 2.310 (1.16), 2.318 (0.54), 2.328 (1.90), 2.347 (1.27), 2.365 (0.41), 2.518 (1.29), 2.523 (0.90), 3.755 (0.79), 3.770 (1.04), 3.786 (0.77), 3.880 (3.65), 3.898 (5.97), 3.917 (3.49), 6.316 (1.30), 6.321 (1.68), 6.348 (6.87), 6.876 (2.00), 6.899 (2.27), 7.435 (1.65), 7.442 (1.71), 7.457 (1.50), 7.464 (1.53), 7.667 (4.19), 7.674 (4.05), 7.841 (1.83); LC-MS (method 7): Rₜ=1.46 min; MS (ESIpos): m/z=466 [M+H]⁺

### Example 50

### 6-(Azetidin-1-yl)-N-[2-(cyclopropylamino)-6-fluorobenzene-1-sulfonyl]-4-fluoro-1-benzofuran-2-carboxamide

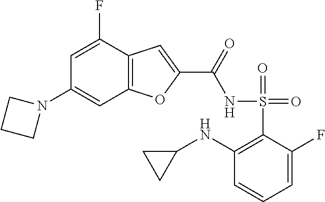 [SMILES: O=C(NS(=O)(=O)c1c(F)cccc1NC1CC1)c1cc2c(F)cc(N3CCC3)cc2o1]

Synthesized and purified analogously to Example 3 using 2-(cyclopropylamino)-6-fluorobenzene-1-sulfonamide (117 mg, 510 μmol) instead of 2-(cyclopentyloxy)-5-ethylbenzene-1-sulfonamide to give 54.0 mg (27% yield) of the title compound as a yellow solid. ¹H-NMR (400 MHz, DMSO-d₆) δ [ppm]: 0.492 (1.52), 0.503 (5.20), 0.509 (5.41), 0.512 (5.77), 0.518 (5.30), 0.528 (1.87), 0.824 (1.68), 0.835 (4.94), 0.840 (6.08), 0.852 (6.00), 0.857 (4.76), 0.869 (1.50), 2.074 (0.44), 2.290 (1.01), 2.307 (3.14), 2.326 (5.04), 2.345 (3.47), 2.363 (1.13), 2.518 (2.03), 2.522 (1.88), 2.532 (1.74), 2.540 (2.25), 2.549 (2.94), 2.558 (1.99), 2.565 (1.45), 2.575 (0.65), 2.668 (0.52), 3.879 (9.75), 3.898 (16.00), 3.916 (9.40), 6.314 (3.34), 6.318 (4.92), 6.335 (7.88), 6.344 (5.52), 6.347 (3.01), 6.495 (2.25), 6.514 (2.46), 6.516 (2.46), 6.521 (2.33), 6.523 (2.34), 6.542 (2.33), 6.983 (4.72), 7.005 (5.07), 7.053 (0.47), 7.427 (1.72), 7.443 (1.99), 7.447 (2.90), 7.464 (2.89), 7.469 (1.95), 7.485 (1.59), 7.938 (6.92); LC-MS (method 3): Rₜ=1.35 min; MS (ESIpos): m/z=448 [M+H]⁺

### Example 51

### 6-(Azetidin-1-yl)-N-[2-(dimethylcarbamoyl)benzene-1-sulfonyl]-4-fluoro-1-benzofuran-2-carboxamide

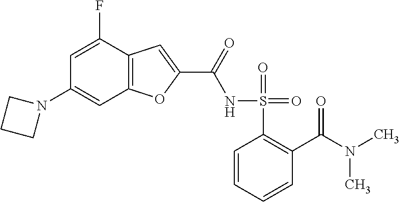 [SMILES: CN(C)C(=O)c1ccccc1S(=O)(=O)NC(=O)c1cc2c(F)cc(N3CCC3)cc2o1]

Synthesized and purified analogously to Example 3 using commercially available N,N-dimethyl-2-sulfamoylbenzamide (CAS: 125274-65-9, 116 mg, 510 μmol) instead of 2-(cyclopentyloxy)-5-ethylbenzene-1-sulfonamide to give 40.0 mg (20% yield) of the title compound as a yellow solid. ¹H-NMR (400 MHz, DMSO-d₆) δ [ppm]: 2.307 (1.02), 2.326 (1.70), 2.345 (1.12), 2.518 (0.83), 2.523 (0.55), 2.743 (14.69), 2.990 (16.00), 3.374 (0.45), 3.879 (3.11), 3.897 (5.09), 3.915 (2.96), 6.319 (1.13), 6.323 (1.42), 6.351 (5.77), 7.397 (1.25), 7.399 (1.33), 7.415 (1.44), 7.418 (1.42), 7.636 (0.65), 7.639 (0.67), 7.655 (1.34), 7.658 (1.28), 7.674 (1.07), 7.677 (0.93), 7.724 (0.98), 7.727 (1.03), 7.743 (1.53), 7.746 (1.52), 7.762 (0.61), 7.765 (0.55), 7.969 (2.01), 8.086 (1.56), 8.089 (1.62), 8.106 (1.43), 8.109 (1.37); LC-MS (method 7): Rₜ=1.07 min; MS (ESIpos): m/z=446 [M+H]⁺

### Example 52

### 6-(Azetidin-1-yl)-4-fluoro-N-[2-(3,3,3-trifluoropropyl)benzene-1-sulfonyl]-1-benzofuran-2-carboxamide

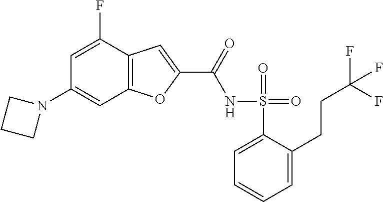 [SMILES: O=C(NS(=O)(=O)c1ccccc1CCC(F)(F)F)c1cc2c(F)cc(N3CCC3)cc2o1]

Synthesized and purified analogously to Example 3 using commercially available 2-(3,3,3-trifluoropropyl)benzene-1-sulfonamide (CAS: 94125-42-5, 129 mg, 510 μmol) instead of 2-(cyclopentyloxy)-5-ethylbenzene-1-sulfonamide to give 36.0 mg (16% yield) of the title compound as a yellow solid. ¹H-NMR (400 MHz, DMSO-d₆) δ [ppm]: 2.289 (1.02), 2.307 (3.25), 2.326 (5.79), 2.337 (1.72), 2.344 (3.80), 2.362 (1.38), 2.518 (4.43), 2.523 (3.10), 2.558 (0.62), 2.571 (0.45), 2.578 (0.68), 2.586 (2.05), 2.599 (1.81), 2.606 (1.85), 2.615 (2.70), 2.627 (3.03), 2.634 (1.83), 2.642 (1.64), 2.655 (2.24), 2.664 (1.50), 2.669 (1.79), 2.673 (1.24), 2.679 (0.64), 2.683 (0.61), 3.275 (4.51), 3.288 (3.10), 3.296 (4.26), 3.303 (3.17), 3.316 (4.14), 3.874 (9.66), 3.893 (16.00), 3.911 (9.52), 3.934 (0.91), 6.313 (3.43), 6.318 (4.49), 6.322 (2.71), 6.343 (12.42), 6.414 (0.62), 7.491 (1.60), 7.494 (1.82), 7.513 (3.74), 7.528 (4.58), 7.546 (4.40), 7.559 (0.45), 7.580 (0.40), 7.638 (2.28), 7.641 (2.35), 7.657 (3.23), 7.675 (1.37), 7.679 (1.31), 7.744 (0.58), 7.752 (1.08), 7.754 (1.12), 7.765 (0.46), 7.889 (2.46), 8.041 (4.42), 8.045 (4.32), 8.062 (4.22), 8.064 (3.82), 8.078 (0.65), 8.098 (0.54); LC-MS (method 7): Rₜ=1.35 min; MS (ESIpos): m/z=471 [M+H]⁺

### Example 53

### 6-(Azetidin-1-yl)-4-fluoro-N-[2-methyl-6-(propan-2-yl)benzene-1-sulfonyl]-1-benzofuran-2-carboxamide

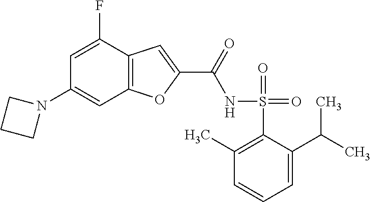 [SMILES: Cc1cccc(C(C)C)c1S(=O)(=O)NC(=O)c1cc2c(F)cc(N3CCC3)cc2o1]

Synthesized and purified analogously to Example 3 using commercially available 2-methyl-6-(propan-2-yl)benzene-1-sulfonamide (CAS: 1094713-96-8, 109 mg, 510 μmol) instead of 2-(cyclopentyloxy)-5-ethylbenzene-1-sulfonamide to give 28.0 mg (15% yield) of the title compound as a yellow solid. ¹H-NMR (400 MHz, DMSO-d₆) δ [ppm]: 1.137 (14.51), 1.154 (14.83), 1.300 (0.52), 1.315 (0.51), 2.283 (0.52), 2.302 (1.60), 2.321 (2.39), 2.332 (0.80), 2.339 (1.73), 2.357 (0.56), 2.518 (0.81), 2.522 (0.53), 2.713 (16.00), 3.389 (0.57), 3.859 (0.71), 3.870 (4.80), 3.889 (7.79), 3.906 (4.55), 4.146 (0.94), 4.163 (1.27), 4.180 (0.91), 6.311 (1.58), 6.316 (2.47), 6.325 (3.05), 6.327 (3.71), 6.342 (2.35), 6.345 (1.54), 7.202 (1.25), 7.206 (1.27), 7.218 (1.53), 7.223 (1.43), 7.453 (0.73), 7.467 (3.41), 7.471 (5.20), 7.488 (2.45), 7.508 (0.68), 7.955 (3.12); LC-MS (method 3): Rₜ=1.39 min; MS (ESIneg): m/z=429 [M−H]⁻

### Example 54

### 6-(Azetidin-1-yl)-N-[2-chloro-5-(ethoxymethyl)benzene-1-sulfonyl]-4-fluoro-1-benzofuran-2-carboxamide

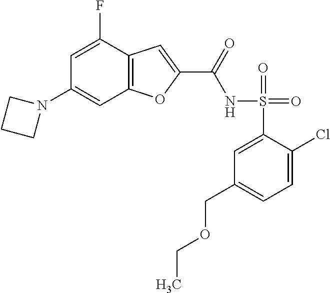 [SMILES: CCOCc1ccc(Cl)c(S(=O)(=O)NC(=O)c2cc3c(F)cc(N4CCC4)cc3o2)c1]

Synthesized and purified analogously to Example 3 using 2-chloro-5-(ethoxymethyl)benzene-1-sulfonamide (127 mg, 510 μmol) instead of 2-(cyclopentyloxy)-5-ethylbenzene-1-sulfonamide to give 50.0 mg (23% yield) of the title compound as a light yellow solid. ¹H-NMR (500 MHz, DMSO-d₆) δ [ppm]: 1.174 (7.76), 1.188 (16.00), 1.201 (7.16), 1.211 (2.71), 1.225 (2.57), 2.296 (0.53), 2.311 (1.58), 2.326 (2.30), 2.341 (1.74), 2.357 (1.09), 2.361 (1.09), 2.365 (0.77), 2.514 (3.03), 2.518 (2.58), 2.522 (1.99), 2.631 (0.73), 2.635 (0.99), 2.638 (0.68), 3.522 (2.24), 3.536 (7.01), 3.550 (7.38), 3.564 (2.29), 3.854 (0.76), 3.869 (1.30), 3.880 (4.55), 3.894 (7.25), 3.909 (4.30), 4.570 (9.11), 6.322 (2.09), 6.344 (7.13), 6.411 (0.44), 7.605 (0.41), 7.628 (3.00), 7.644 (0.50), 7.965 (0.42), 8.099 (2.54); LC-MS (method 7): Rₜ=1.30 min; MS (ESIpos): m/z=467 [M+H]⁺

### Example 55

### 6-(Azetidin-1-yl)-4-fluoro-N-{2-[(1E)-3,3,3-trifluoroprop-1-en-1-yl]benzene-1-sulfonyl}-1-benzofuran-2-carboxamide

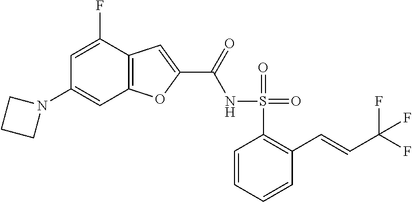 [SMILES: O=C(NS(=O)(=O)c1ccccc1/C=C/C(F)(F)F)c1cc2c(F)cc(N3CCC3)cc2o1]

Synthesized and purified analogously to Example 3 using commercially available 2-[(1E)-3,3,3-trifluoroprop-1-en-1-yl]benzene-1-sulfonamide (CAS: 90300-44-0, 128 mg, 510 μmol) instead of 2-(cyclopentyloxy)-5-ethylbenzene-1-sulfonamide to give 55.0 mg (26% yield) of the title compound as a yellow solid. ¹H-NMR (400 MHz, DMSO-d₆) δ [ppm]: 1.208 (0.79), 1.225 (0.79), 2.074 (5.23), 2.283 (1.04), 2.300 (3.19), 2.319 (4.75), 2.338 (3.62), 2.356 (1.18), 2.518 (1.76), 2.522 (1.15), 2.668 (0.47), 3.847 (0.44), 3.868 (9.93), 3.886 (16.00), 3.905 (9.45), 6.304 (3.22), 6.307 (4.83), 6.322 (8.10), 6.333 (5.05), 6.337 (3.01), 6.713 (0.93), 6.731 (2.74), 6.748 (2.65), 6.753 (1.24), 6.765 (0.81), 6.770 (3.12), 6.788 (2.85), 6.805 (0.72), 7.674 (1.54), 7.676 (1.64), 7.693 (3.72), 7.695 (3.74), 7.712 (2.86), 7.715 (2.79), 7.742 (2.10), 7.745 (2.32), 7.761 (3.60), 7.764 (3.84), 7.780 (1.78), 7.782 (1.67), 7.860 (7.44), 7.880 (3.49), 8.076 (2.39), 8.081 (2.43), 8.111 (5.47), 8.114 (6.60), 8.121 (2.55), 8.130 (4.44), 8.134 (4.13); LC-MS (method 3): Rₜ=1.31 min; MS (ESIpos): m/z=469 [M+H]⁺

### Example 56

### 6-(Azetidin-1-yl)-4-fluoro-N-(quinoxaline-5-sulfonyl)-1-benzofuran-2-carboxamide

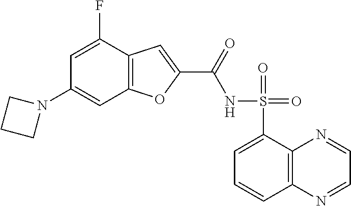 [SMILES: O=C(NS(=O)(=O)c1cccc2nccnc12)c1cc2c(F)cc(N3CCC3)cc2o1]

Synthesized and purified analogously to Example 3 using commercially available quinoxaline-5-sulfonamide (CAS: 1995073-91-0, 107 mg, 510 μmol) instead of 2-(cyclopentyloxy)-5-ethylbenzene-1-sulfonamide to give 5.00 mg (2% yield) of the title compound as a yellow solid. ¹H-NMR (400 MHz, DMSO-d₆) δ [ppm]: 2.074 (0.58), 2.272 (0.79), 2.290 (2.53), 2.308 (3.82), 2.327 (4.70), 2.344 (1.36), 2.522 (16.00), 2.665 (1.14), 2.669 (1.68), 2.673 (1.31), 3.850 (7.35), 3.868 (12.59), 3.886 (7.79), 3.909 (0.82), 6.297 (13.19), 6.326 (3.47), 6.329 (3.05), 6.639 (1.50), 8.048 (2.02), 8.070 (3.47), 8.081 (1.13), 8.089 (4.08), 8.090 (4.27), 8.102 (1.01), 8.109 (3.71), 8.154 (0.72), 8.175 (0.64), 8.448 (3.02), 8.451 (3.36), 8.469 (2.88), 8.472 (2.96), 8.599 (3.54), 8.603 (3.70), 8.618 (3.50), 8.622 (3.27), 9.069 (5.08), 9.074 (8.65), 9.084 (9.55), 9.088 (5.95); LC-MS (method 3): Rₜ=1.09 min; MS (ESIpos): m/z=427 [M+H]⁺

### Example 57

### Methyl (5-{[6-(azetidin-1-yl)-4-fluoro-1-benzofuran-2-carbonyl]sulfamoyl}naphthalen-2-yl)acetate

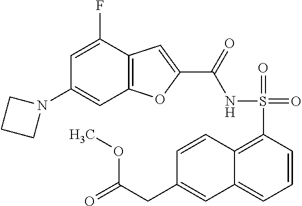 [SMILES: COC(=O)Cc1ccc2c(S(=O)(=O)NC(=O)c3cc4c(F)cc(N5CCC5)cc4o3)cccc2c1]

Synthesized and purified analogously to Example 3 using methyl (5-sulfamoylnaphthalen-2-yl)acetate (142 mg, 510 μmol) instead of 2-(cyclopentyloxy)-5-ethylbenzene-1-sulfonamide to give 18.0 mg (8% yield) of the title compound as a yellow solid. ¹H-NMR (400 MHz, DMSO-d₆) δ [ppm]: 1.183 (0.57), 1.191 (0.42), 1.199 (0.55), 2.074 (0.70), 2.266 (0.84), 2.284 (2.66), 2.303 (3.96), 2.321 (3.48), 2.338 (1.31), 2.522 (4.98), 2.539 (2.81), 2.643 (0.58), 2.669 (0.53), 2.673 (0.40), 3.423 (0.96), 3.631 (2.24), 3.640 (0.78), 3.849 (7.79), 3.868 (13.28), 3.885 (8.65), 3.898 (16.00), 3.938 (0.83), 6.286 (2.38), 6.290 (4.41), 6.300 (6.99), 6.316 (4.25), 6.320 (3.33), 7.648 (2.95), 7.653 (3.20), 7.671 (3.16), 7.676 (3.47), 7.713 (3.48), 7.733 (4.81), 7.752 (3.68), 7.895 (0.42), 7.913 (5.03), 7.935 (0.52), 7.940 (0.52), 7.993 (5.10), 7.996 (5.35), 8.262 (3.51), 8.283 (3.37), 8.348 (4.03), 8.351 (4.20), 8.366 (4.01), 8.370 (3.89), 8.632 (4.60), 8.655 (4.42); LC-MS (method 3): Rₜ=1.24 min; MS (ESIneg): m/z=495 [M−H]⁻

### Example 58

### 6-(Azetidin-1-yl)-4-fluoro-N-[2-propyl-6-(trifluoromethyl)benzene-1-sulfonyl]-1-benzofuran-2-carboxamide

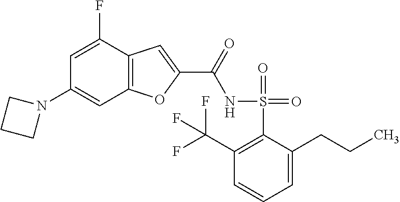 [SMILES: CCCc1cccc(C(F)(F)F)c1S(=O)(=O)NC(=O)c1cc2c(F)cc(N3CCC3)cc2o1]

Synthesized and purified analogously to Example 3 using 2-propyl-6-(trifluoromethyl)benzene-1-sulfonamide (136 mg, 510 μmol) instead of 2-(cyclopentyloxy)-5-ethylbenzene-1-sulfonamide to give 40.0 mg (17% yield) of the title compound as a light yellow solid. ¹H-NMR (400 MHz, DMSO-d₆) δ [ppm]: 0.899 (6.68), 0.917 (16.00), 0.936 (7.38), 1.302 (0.94), 1.317 (0.95), 1.482 (0.55), 1.500 (1.95), 1.519 (2.97), 1.526 (1.78), 1.532 (1.72), 1.539 (2.90), 1.558 (1.83), 1.576 (0.44), 2.288 (0.95), 2.306 (2.99), 2.326 (5.85), 2.336 (1.94), 2.344 (3.28), 2.362 (1.01), 2.518 (7.64), 2.522 (4.77), 2.660 (0.64), 2.664 (1.43), 2.669 (2.03), 2.673 (1.46), 2.678 (0.66), 3.103 (3.97), 3.116 (3.02), 3.123 (3.97), 3.129 (3.10), 3.143 (3.81), 3.843 (0.69), 3.861 (1.27), 3.875 (8.86), 3.893 (14.55), 3.911 (8.33), 6.315 (4.00), 6.326 (7.14), 6.341 (3.78), 6.345 (2.80), 7.088 (0.59), 7.090 (0.60), 7.721 (0.64), 7.741 (2.22), 7.758 (6.02), 7.777 (1.25), 7.843 (3.05), 7.849 (3.09), 7.861 (2.89); LC-MS (method 7): Rₜ=1.42 min; MS (ESIpos): m/z=485 [M+H]⁺

### Example 59

### 6-(Azetidin-1-yl)-N-[2-chloro-5-(2-methoxyethoxy)benzene-1-sulfonyl]-4-fluoro-1-benzofuran-2-carboxamide

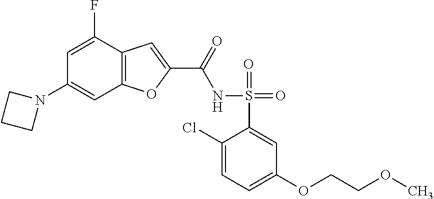 [SMILES: COCCOc1ccc(Cl)c(S(=O)(=O)NC(=O)c2cc3c(F)cc(N4CCC4)cc3o2)c1]

Synthesized and purified analogously to Example 3 using 2-chloro-5-(2-methoxyethoxy)benzene-1-sulfonamide (136 mg, 510 μmol) instead of 2-(cyclopentyloxy)-5-ethylbenzene-1-sulfonamide to give 37.0 mg (16% yield) of the title compound as a yellows solid. ¹H-NMR (400 MHz, DMSO-d₆) δ [ppm]: 2.074 (1.70), 2.288 (1.02), 2.306 (3.13), 2.325 (4.68), 2.343 (3.44), 2.361 (1.12), 2.518 (1.78), 2.522 (1.13), 2.669 (0.48), 3.140 (0.42), 3.492 (0.58), 3.677 (5.52), 3.684 (4.83), 3.687 (6.03), 3.692 (4.89), 3.699 (6.10), 3.843 (1.12), 3.860 (5.40), 3.877 (9.77), 3.895 (16.00), 3.913 (9.33), 4.185 (5.44), 4.192 (4.46), 4.196 (5.73), 4.200 (4.61), 4.207 (5.09), 6.316 (3.58), 6.321 (4.60), 6.347 (14.57), 7.282 (0.42), 7.293 (3.62), 7.302 (3.71), 7.316 (4.17), 7.323 (4.29), 7.559 (9.59), 7.571 (1.24), 7.580 (7.77), 7.593 (0.95), 7.613 (9.39), 7.621 (9.08), 8.011 (6.32); LC-MS (method 3): Rₜ=1.22 min; MS (ESIpos): m/z=483 [M+H]⁺

### Example 60

### 6-(Azetidin-1-yl)-N-(2-chloro-5-methylpyridine-3-sulfonyl)-4-fluoro-1-benzofuran-2-carboxamide

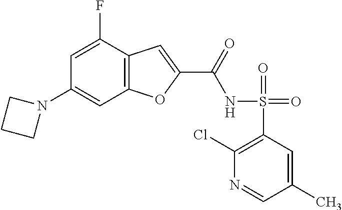 [SMILES: Cc1cnc(Cl)c(S(=O)(=O)NC(=O)c2cc3c(F)cc(N4CCC4)cc3o2)c1]

Synthesized and purified analogously to Example 3 using commercially available 2-chloro-5-methylpyridine-3-sulfonamide (CAS: 1208081-46-2, 81.6 mg, 395 μmol) instead of 2-(cyclopentyloxy)-5-ethylbenzene-1-sulfonamide to give 95.0 mg (65% yield) of the title compound as a yellow solid. ¹H-NMR (400 MHz, DMSO-d₆) δ [ppm]: 2.083 (16.00), 2.308 (0.65), 2.326 (1.07), 2.344 (0.70), 2.424 (4.92), 3.877 (1.72), 3.895 (2.87), 3.913 (1.60), 6.317 (0.70), 6.344 (2.56), 7.924 (0.49), 8.391 (0.98), 8.519 (0.94); LC-MS (method 2): Rₜ=0.75 min; MS (ESIpos): m/z=424 [M+H]⁺

### Example 61

### 6-(Azetidin-1-yl)-N-(2-cyclopentyl-6-methylbenzene-1-sulfonyl)-4-fluoro-1-benzofuran-2-carboxamide

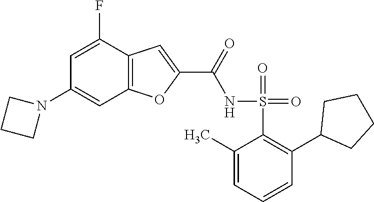 [SMILES: Cc1cccc(C2CCCC2)c1S(=O)(=O)NC(=O)c1cc2c(F)cc(N3CCC3)cc2o1]

Synthesized and purified analogously to Example 3 using 2-cyclopentyl-6-methylbenzene-1-sulfonamide (122 mg, 510 μmol) instead of 2-(cyclopentyloxy)-5-ethylbenzene-1-sulfonamide to give 20.0 mg (10% yield) of the title compound as an offwhite solid. ¹H-NMR (500 MHz, DMSO-d₆) δ [ppm]: 1.477 (1.02), 1.652 (1.38), 1.662 (1.28), 1.676 (0.91), 1.792 (1.54), 1.808 (0.81), 1.919 (1.14), 2.074 (0.57), 2.295 (0.55), 2.309 (1.67), 2.324 (2.40), 2.339 (1.78), 2.368 (0.40), 2.514 (3.23), 2.518 (3.10), 2.522 (2.53), 2.627 (0.41), 2.709 (16.00), 3.877 (5.03), 3.892 (8.20), 3.906 (4.82), 4.129 (0.42), 4.146 (0.60), 4.162 (0.41), 6.322 (2.06), 6.329 (4.08), 6.343 (1.75), 7.184 (1.11), 7.199 (1.18), 7.411 (0.81), 7.426 (1.58), 7.447 (0.98), 7.462 (1.15), 7.477 (0.43), 7.938 (0.45); LC-MS (method 7): Rₜ=1.48 min; MS (ESIpos): m/z=457 [M+H]⁺

### Example 62

### 2-{[6-(Azetidin-1-yl)-4-fluoro-1-benzofuran-2-carbonyl]sulfamoyl}phenyl methyl(phenyl)carbamate

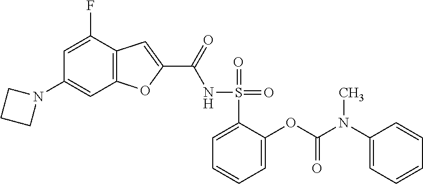 [SMILES: CN(C(=O)Oc1ccccc1S(=O)(=O)NC(=O)c1cc2c(F)cc(N3CCC3)cc2o1)c1ccccc1]

Synthesized and purified analogously to Example 3 using 2-sulfamoylphenyl methyl(phenyl)carbamate (75.0 mg, 245 μmol) instead of 2-(cyclopentyloxy)-5-ethylbenzene-1-sulfonamide to give 55.0 mg (49% yield) of the title compound as a yellow solid. ¹H-NMR (400 MHz, DMSO-d₆) δ [ppm]: 1.210 (0.62), 1.227 (0.60), 2.292 (1.15), 2.310 (3.56), 2.318 (1.87), 2.327 (5.89), 2.347 (3.89), 2.365 (1.23), 2.518 (4.72), 2.523 (3.10), 2.539 (0.56), 2.660 (0.41), 2.665 (0.92), 2.669 (1.34), 2.673 (0.94), 2.678 (0.40), 3.876 (9.48), 3.894 (16.00), 3.913 (9.07), 6.320 (5.01), 6.328 (7.14), 6.346 (3.77), 7.136 (1.25), 7.249 (4.04), 7.268 (7.37), 7.287 (4.09), 7.388 (1.72), 7.468 (1.75), 7.487 (3.00), 7.506 (1.92), 7.719 (1.45), 7.737 (2.24), 7.755 (1.26), 7.829 (0.57), 8.021 (1.82), 8.039 (1.71); LC-MS (method 7): Rₜ=1.28 min; MS (ESIpos): m/z=523 [M+H]⁺

### Example 63

### 6-(Azetidin-1-yl)-4-fluoro-N-[5-(hydroxymethyl)-2-(trifluoromethoxy)benzene-1-sulfonyl]-1-benzofuran-2-carboxamide

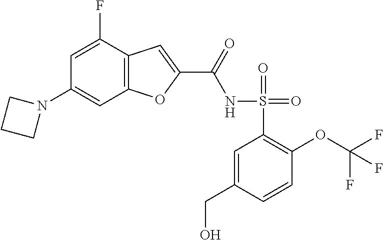 [SMILES: O=C(NS(=O)(=O)c1cc(CO)ccc1OC(F)(F)F)c1cc2c(F)cc(N3CCC3)cc2o1]

Synthesized and purified analogously to Example 3 using commercially available 5-(hydroxymethyl)-2-(trifluoromethoxy)benzene-1-sulfonamide (CAS: 1645275-32-6, 75.0 mg, 277 μmol) instead of 2-(cyclopentyloxy)-5-ethylbenzene-1-sulfonamide to give 50.0 mg (42% yield) of the title compound as a yellow solid. ¹H-NMR (400 MHz, DMSO-d₆) δ [ppm]: 1.743 (0.55), 2.075 (4.96), 2.289 (0.88), 2.307 (2.73), 2.318 (1.29), 2.326 (4.80), 2.332 (2.21), 2.337 (1.35), 2.345 (3.01), 2.363 (0.96), 2.518 (3.56), 2.523 (2.53), 2.665 (0.76), 2.669 (1.08), 2.673 (0.74), 3.086 (0.53), 3.097 (0.53), 3.438 (0.81), 3.877 (8.53), 3.884 (2.05), 3.895 (13.88), 3.913 (8.11), 4.617 (16.00), 6.319 (2.97), 6.323 (3.73), 6.342 (4.35), 6.345 (8.07), 6.349 (9.22), 6.354 (1.88), 7.532 (0.83), 7.537 (2.00), 7.541 (1.98), 7.553 (1.12), 7.558 (2.51), 7.562 (2.39), 7.719 (2.55), 7.725 (2.67), 7.740 (2.11), 7.746 (2.14), 7.952 (2.42), 8.089 (5.49), 8.095 (5.22); LC-MS (method 7): Rₜ=1.13 min; MS (ESIpos): m/z=489 [M+H]⁺

### Example 64

### 6-(Azetidin-1-yl)-4-fluoro-N-(2-hydroxy-6-methylbenzene-1-sulfonyl)-1-benzofuran-2-carboxamide

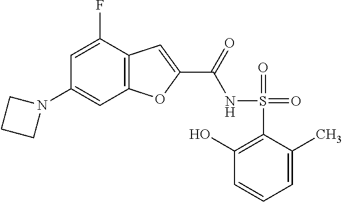 [SMILES: Cc1cccc(O)c1S(=O)(=O)NC(=O)c1cc2c(F)cc(N3CCC3)cc2o1]

Synthesized and purified analogously to Example 3 using commercially available 2-hydroxy-6-methylbenzene-1-sulfonamide (CAS: 43059-22-9, 75.0 mg, 401 μmol) instead of 2-(cyclopentyloxy)-5-ethylbenzene-1-sulfonamide to give 35.0 mg (25% yield) of the title compound as a yellow solid. ¹H-NMR (400 MHz, DMSO-d₆) δ [ppm]: 2.288 (0.54), 2.306 (1.65), 2.326 (2.65), 2.336 (0.84), 2.344 (1.81), 2.361 (0.59), 2.518 (2.34), 2.523 (1.62), 2.539 (0.68), 2.582 (16.00), 2.665 (0.49), 2.669 (0.71), 2.673 (0.49), 3.872 (5.20), 3.890 (8.40), 3.908 (4.85), 6.303 (1.90), 6.307 (2.15), 6.332 (1.54), 6.337 (2.53), 6.345 (3.68), 6.347 (4.23), 6.351 (2.58), 6.765 (1.92), 6.786 (3.38), 6.808 (2.18), 7.276 (1.80), 7.295 (2.50), 7.315 (1.48), 7.927 (0.90); LC-MS (method 7): Rₜ=1.23 min; MS (ESIpos): m/z=405 [M+H]⁺

### Example 65

### 6-(Azetidin-1-yl)-4-fluoro-N-(5-methoxynaphthalene-1-sulfonyl)-1-benzofuran-2-carboxamide

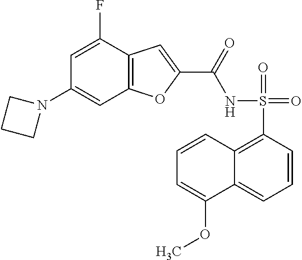 [SMILES: COc1cccc2c(S(=O)(=O)NC(=O)c3cc4c(F)cc(N5CCC5)cc4o3)cccc12]

Synthesized and purified analogously to Example 3 using 5-methoxynaphthalene-1-sulfonamide (121 mg, 510 μmol) instead of 2-(cyclopentyloxy)-5-ethylbenzene-1-sulfonamide to give 30.0 mg (14% yield) of the title compound as an yellow solid. ¹H-NMR (400 MHz, DMSO-d₆) δ [ppm]: 2.284 (1.19), 2.303 (1.76), 2.322 (1.53), 2.331 (0.50), 2.339 (0.55), 2.518 (2.57), 2.522 (1.85), 2.539 (0.63), 3.848 (3.58), 3.866 (6.03), 3.885 (3.65), 3.985 (16.00), 6.286 (1.09), 6.290 (2.00), 6.299 (3.09), 6.316 (1.90), 6.320 (1.47), 7.108 (2.03), 7.127 (2.19), 7.638 (1.54), 7.658 (1.85), 7.660 (2.07), 7.679 (1.56), 7.691 (1.87), 7.709 (2.13), 7.712 (2.22), 7.730 (1.86), 7.917 (2.71), 8.221 (2.15), 8.243 (1.94), 8.394 (2.01), 8.398 (2.29), 8.413 (1.95), 8.416 (1.96), 8.528 (1.79), 8.549 (1.69); LC-MS (method 3): Rₜ=1.30 min; MS (ESIpos): m/z=455 [M+H]⁺

### Example 66

### 6-(Azetidin-1-yl)-N-[5-(dimethylamino)naphthalene-1-sulfonyl]-4-fluoro-1-benzofuran-2-carboxamide

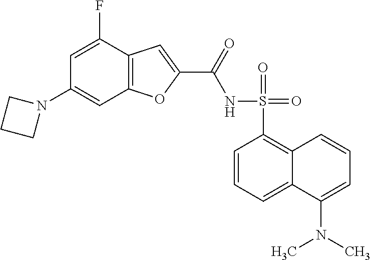 [SMILES: CN(C)c1cccc2c(S(=O)(=O)NC(=O)c3cc4c(F)cc(N5CCC5)cc4o3)cccc12]

Synthesized and purified analogously to Example 3 using commercially available 5-(dimethylamino)naphthalene-1-sulfonamide (CAS: 1431-39-6, 128 mg, 510 μmol) instead of 2-(cyclopentyloxy)-5-ethylbenzene-1-sulfonamide to give 26.0 mg (12% yield) of the title compound as a yellow solid. ¹H-NMR (400 MHz, DMSO-d₆) δ [ppm]: 2.285 (0.60), 2.304 (0.89), 2.322 (0.79), 2.518 (1.44), 2.522 (1.05), 2.815 (16.00), 3.849 (1.77), 3.868 (2.99), 3.885 (1.86), 6.288 (0.53), 6.292 (1.04), 6.298 (1.29), 6.301 (1.56), 6.304 (1.24), 6.318 (0.99), 6.322 (0.89), 7.231 (0.91), 7.248 (0.99), 7.602 (0.80), 7.621 (0.83), 7.624 (0.98), 7.643 (0.72), 7.697 (0.85), 7.715 (0.99), 7.717 (1.02), 7.736 (0.91), 7.922 (1.21), 8.316 (0.95), 8.337 (0.89), 8.366 (0.98), 8.369 (1.11), 8.384 (0.96), 8.387 (0.98), 8.526 (0.86), 8.547 (0.83); LC-MS (method 3): Rₜ=1.33 min; MS (ESIpos): m/z=468 [M+H]⁺

### Example 67

### 6-(Azetidin-1-yl)-4-fluoro-N-(3-methoxypyridine-2-sulfonyl)-1-benzofuran-2-carboxamide

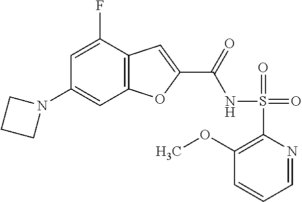 [SMILES: COc1cccnc1S(=O)(=O)NC(=O)c1cc2c(F)cc(N3CCC3)cc2o1]

Synthesized and purified analogously to Example 3 using commercially available 3-methoxypyridine-2-sulfonamide (CAS: 1566809-26-4, 125 mg, 663 μmol) instead of 2-(cyclopentyloxy)-5-ethylbenzene-1-sulfonamide to give 80.0 mg (32% yield) of the title compound as a yellow solid. ¹H-NMR (400 MHz, DMSO-d₆) δ [ppm]: 2.074 (0.90), 2.317 (1.29), 2.327 (0.74), 2.335 (1.84), 2.354 (1.30), 2.372 (0.42), 2.518 (1.06), 2.523 (0.65), 2.539 (0.47), 3.872 (0.80), 3.886 (3.72), 3.904 (5.93), 3.922 (3.83), 3.930 (16.00), 6.332 (1.58), 6.337 (1.53), 6.362 (1.18), 6.366 (1.68), 6.382 (2.35), 6.384 (2.78), 6.415 (0.42), 7.532 (0.79), 7.535 (0.78), 7.684 (1.33), 7.694 (1.41), 7.705 (1.89), 7.716 (1.99), 7.797 (1.75), 7.799 (1.96), 7.818 (1.29), 7.821 (1.28), 7.999 (1.94), 8.196 (2.14), 8.199 (2.23), 8.207 (2.13), 8.210 (1.95); LC-MS method 7): Rₜ=0.96 min; MS (ESIpos): m/z=406 [M+H]⁺

### Example 68

### 6-(Azetidin-1-yl)-N-[4-(2-cyanoethyl)benzene-1-sulfonyl]-4-fluoro-1-benzofuran-2-carboxamide

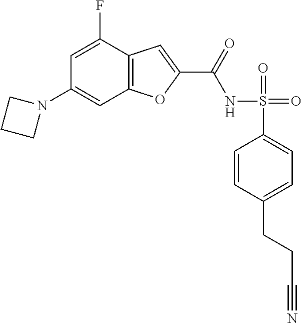 [SMILES: N#CCCc1ccc(S(=O)(=O)NC(=O)c2cc3c(F)cc(N4CCC4)cc3o2)cc1]

Synthesized and purified analogously to Example 3 using commercially available 4-(2-cyanoethyl)benzene-1-sulfonamide (CAS: 88961-77-7, 107 mg, 510 μmol) instead of 2-(cyclopentyloxy)-5-ethylbenzene-1-sulfonamide to give 50.0 mg (26% yield) of the title compound as a yellow solid. ¹H-NMR (400 MHz, DMSO-d₆) δ [ppm]: 2.074 (0.45), 2.282 (1.01), 2.301 (3.16), 2.319 (4.73), 2.337 (3.77), 2.355 (1.33), 2.522 (2.03), 2.539 (1.02), 2.855 (2.94), 2.858 (3.04), 2.876 (10.50), 2.893 (6.19), 2.976 (5.25), 2.994 (7.80), 3.012 (3.02), 3.868 (9.58), 3.886 (16.00), 3.905 (9.60), 6.298 (3.60), 6.302 (4.43), 6.327 (2.60), 6.331 (6.96), 6.334 (8.80), 6.336 (9.17), 7.557 (9.53), 7.578 (10.81), 7.886 (10.53), 7.889 (11.00), 7.940 (1.70), 7.945 (12.83), 7.949 (4.58), 7.961 (3.93), 7.966 (11.69); LC-MS (method 3): Rₜ=1.12 min; MS (ESIneg): m/z=426 [M−H]⁻

### Example 69

### 6-(Azetidin-1-yl)-N-(3-bromopyridine-2-sulfonyl)-4-fluoro-1-benzofuran-2-carboxamide

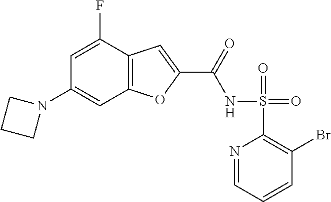 [SMILES: O=C(NS(=O)(=O)c1ncccc1Br)c1cc2c(F)cc(N3CCC3)cc2o1]

Synthesized and purified analogously to Example 3 using commercially available 3-bromopyridine-2-sulfonamide (CAS: 65938-87-6, 121 mg, 510 μmol) instead of 2-(cyclopentyloxy)-5-ethylbenzene-1-sulfonamide to give 8.00 mg (4% yield) of the title compound as a yellow solid. ¹H-NMR (400 MHz, DMSO-d₆) δ [ppm]: 2.074 (11.46), 2.304 (1.02), 2.322 (3.58), 2.331 (1.88), 2.340 (4.74), 2.359 (3.67), 2.377 (1.33), 2.518 (8.19), 2.522 (5.85), 2.665 (0.49), 2.669 (0.71), 2.673 (0.54), 3.872 (0.55), 3.912 (16.00), 3.930 (9.68), 6.342 (3.67), 6.345 (4.33), 6.371 (3.30), 6.374 (4.65), 6.388 (6.53), 6.391 (7.69), 7.620 (5.27), 7.631 (5.26), 7.640 (5.38), 7.652 (5.60), 7.987 (6.74), 8.404 (5.21), 8.408 (5.78), 8.424 (5.25), 8.428 (5.21), 8.646 (5.64), 8.649 (6.57), 8.657 (6.15), 8.661 (5.86); LC-MS (method 3): Rₜ=1.05 min; MS (ESIpos): m/z=456 [M+H]⁺

### Example 70

### 6-(Azetidin-1-yl)-4-fluoro-N-{2-[(3-methyl-1-phenyl-1H-pyrazol-5-yl)amino]benzene-1-sulfonyl}-1-benzofuran-2-carboxamide

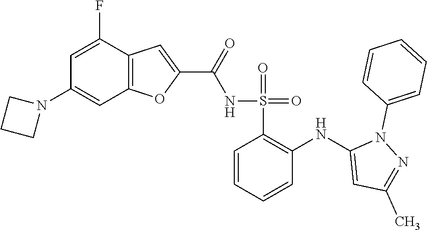 [SMILES: Cc1cc(Nc2ccccc2S(=O)(=O)NC(=O)c2cc3c(F)cc(N4CCC4)cc3o2)n(-c2ccccc2)n1]

Synthesized and purified analogously to Example 3 using 2-[(3-methyl-1-phenyl-1H-pyrazol-5-yl)amino]benzene-1-sulfonamide (168 mg, 510 μmol) instead of 2-(cyclopentyloxy)-5-ethylbenzene-1-sulfonamide to give 41.0 mg (17% yield) of the title compound as a yellow solid. ¹H-NMR (400 MHz, DMSO-d₆) δ [ppm]: 2.232 (16.00), 2.296 (0.52), 2.314 (1.50), 2.332 (2.32), 2.350 (1.73), 2.368 (0.64), 2.539 (1.08), 3.435 (0.44), 3.908 (7.02), 3.927 (4.22), 6.233 (5.54), 6.334 (1.51), 6.338 (1.94), 6.364 (6.61), 6.685 (2.20), 6.706 (2.28), 6.884 (1.13), 6.886 (1.16), 6.905 (2.25), 6.922 (1.31), 6.925 (1.28), 7.025 (0.64), 7.037 (0.50), 7.044 (2.17), 7.049 (0.85), 7.062 (1.94), 7.092 (2.90), 7.112 (4.13), 7.124 (0.97), 7.129 (1.82), 7.358 (0.97), 7.362 (1.05), 7.380 (1.77), 7.398 (0.94), 7.402 (0.97), 7.469 (3.83), 7.488 (4.03), 7.491 (2.87), 7.775 (3.25), 7.788 (2.66), 7.811 (2.25), 7.814 (2.34), 7.831 (2.07), 7.835 (2.04); LC-MS (method 3): Rₜ=1.35 min; MS (ESIpos): m/z=546 [M+H]⁺

### Example 71

### 6-(Azetidin-1-yl)-4-fluoro-N-(3-hydroxynaphthalene-1-sulfonyl)-1-benzofuran-2-carboxamide

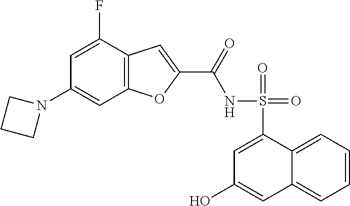 [SMILES: O=C(NS(=O)(=O)c1cc(O)cc2ccccc12)c1cc2c(F)cc(N3CCC3)cc2o1]

Synthesized and purified analogously to Example 3 using 3-hydroxynaphthalene-1-sulfonamide (75.0 mg, 336 μmol) instead of 2-(cyclopentyloxy)-5-ethylbenzene-1-sulfonamide to give 3.00 mg (75% purity, 2% yield) of the title compound as a yellow solid. ¹H-NMR (400 MHz, DMSO-d6) δ[ppm]: 1.715 (0.48), 1.851 (1.70), 1.866 (4.52), 1.868 (4.50), 1.883 (1.74), 2.291 (0.99), 2.310 (1.42), 2.318 (1.55), 2.322 (2.59), 2.327 (3.83), 2.332 (2.68), 2.336 (1.19), 2.518 (10.44), 2.523 (7.01), 2.539 (16.00), 2.660 (0.88), 2.665 (2.02), 2.669 (2.93), 2.673 (2.09), 2.678 (0.90), 3.817 (1.05), 3.836 (1.79), 3.852 (3.25), 3.870 (4.20), 3.888 (2.39), 6.283 (0.79), 6.316 (3.87), 7.439 (0.91), 7.468 (0.68), 7.489 (0.68), 7.594 (0.48), 7.611 (0.46), 7.619 (0.48), 7.839 (0.70), 7.860 (0.61), 7.967 (1.07), 8.046 (0.48), 8.544 (0.81), 8.565 (0.74); LC-MS (method 7): Rₜ=1.15 min; MS (ESIpos): m/z=441 [M+H]⁺

### Example 72

### 6-(Azetidin-1-yl)-N-[2-chloro-5-(dimethylsulfamoyl)benzene-1-sulfonyl]-4-fluoro-1-benzofuran-2-carboxamide

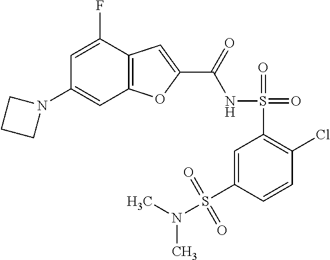 [SMILES: CN(C)S(=O)(=O)c1ccc(Cl)c(S(=O)(=O)NC(=O)c2cc3c(F)cc(N4CCC4)cc3o2)c1]

Synthesized and purified analogously to Example 3 using commercially available 4-chloro-N¹,N¹-dimethylbenzene-1,3-disulfonamide (CAS: 1094537-40-2, 152 mg, 510 μmol) instead of 2-(cyclopentyloxy)-5-ethylbenzene-1-sulfonamide to give 43.0 mg (19% yield) of the title compound as a yellow solid. ¹H-NMR (400 MHz, DMSO-d₆) δ [ppm]: 2.074 (0.95), 2.304 (0.48), 2.323 (0.73), 2.342 (0.53), 2.518 (0.40), 2.681 (16.00), 3.872 (1.52), 3.890 (2.44), 3.908 (1.40), 6.309 (0.56), 6.313 (0.67), 6.338 (0.42), 6.342 (0.89), 6.347 (1.19), 6.349 (1.33), 7.901 (0.60), 7.932 (0.94), 7.953 (1.51), 8.008 (0.80), 8.014 (0.82), 8.029 (0.47), 8.034 (0.51), 8.371 (1.56), 8.376 (1.55); LC-MS (method 3): Rₜ=1.17 min; MS (ESIpos): m/z=516 [M+H]⁺

### Example 73

### 6-(Azetidin-1-yl)-4-fluoro-N-[2-(morpholine-4-carbonyl)benzene-1-sulfonyl]-1-benzofuran-2-carboxamide

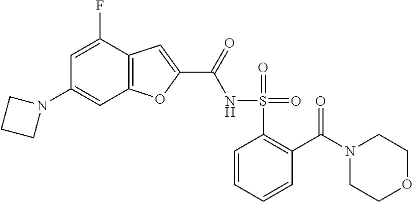 [SMILES: O=C(NS(=O)(=O)c1ccccc1C(=O)N1CCOCC1)c1cc2c(F)cc(N3CCC3)cc2o1]

Synthesized and purified analogously to Example 3 using 2-(morpholine-4-carbonyl)benzene-1-sulfonamide (138 mg, 510 μmol) instead of 2-(cyclopentyloxy)-5-ethylbenzene-1-sulfonamide to give 46.0 mg (21% yield) of the title compound as a yellow solid. ¹H-NMR (400 MHz, DMSO-d₆) δ [ppm]: 2.073 (5.07), 2.284 (0.88), 2.302 (2.72), 2.321 (3.97), 2.332 (1.24), 2.339 (3.00), 2.357 (0.97), 2.518 (0.77), 2.522 (0.52), 3.123 (2.03), 3.137 (3.96), 3.151 (2.45), 3.176 (0.42), 3.483 (0.87), 3.493 (1.23), 3.511 (1.75), 3.522 (2.23), 3.535 (1.29), 3.551 (1.41), 3.565 (2.38), 3.575 (1.64), 3.594 (1.38), 3.605 (0.98), 3.612 (1.12), 3.626 (3.19), 3.642 (5.64), 3.653 (4.31), 3.663 (6.08), 3.874 (8.21), 3.892 (13.52), 3.910 (7.83), 6.313 (3.14), 6.316 (3.96), 6.344 (16.00), 7.452 (3.52), 7.456 (3.84), 7.471 (4.22), 7.475 (4.15), 7.648 (1.92), 7.651 (1.88), 7.667 (3.86), 7.670 (3.60), 7.687 (3.10), 7.691 (2.78), 7.735 (3.12), 7.738 (3.29), 7.754 (4.57), 7.757 (4.60), 7.772 (1.80), 7.775 (1.67), 7.984 (8.99), 8.085 (4.35), 8.087 (4.52), 8.105 (4.05), 8.107 (3.89); LC-MS (method 3): Rₜ=1.09 min; MS (ESIpos): m/z=488 [M+H]⁺

### Example 74

### Methyl (2E)-3-(2-{[6-(azetidin-1-yl)-4-fluoro-1-benzofuran-2-carbonyl]sulfamoyl}phenyl)prop-2-enoate

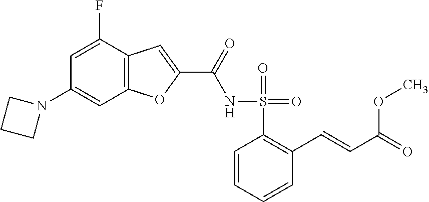 [SMILES: COC(=O)/C=C/c1ccccc1S(=O)(=O)NC(=O)c1cc2c(F)cc(N3CCC3)cc2o1]

Synthesized and purified analogously to Example 3 using methyl (2E)-3-(2-sulfamoylphenyl)prop-2-enoate (75.0 mg, 311 μmol) instead of 2-(cyclopentyloxy)-5-ethylbenzene-1-sulfonamide to give 28.0 mg (21% yield) of the title compound as a yellow solid. ¹H-NMR (400 MHz, DMSO-d₆) δ [ppm]: 2.074 (5.48), 2.283 (1.09), 2.301 (3.27), 2.320 (4.86), 2.331 (2.16), 2.338 (3.88), 2.356 (1.43), 2.522 (5.79), 2.539 (3.55), 2.664 (0.42), 2.669 (0.59), 2.673 (0.46), 2.838 (1.55), 3.475 (0.58), 3.545 (0.74), 3.835 (0.56), 3.854 (0.96), 3.869 (9.60), 3.887 (16.00), 3.905 (9.72), 6.293 (0.72), 6.305 (3.07), 6.310 (4.73), 6.326 (8.04), 6.336 (5.58), 6.340 (3.72), 6.526 (9.46), 6.565 (9.93), 7.654 (1.45), 7.657 (1.68), 7.673 (3.79), 7.676 (4.02), 7.692 (3.20), 7.695 (3.22), 7.712 (2.27), 7.716 (2.65), 7.731 (3.51), 7.734 (3.88), 7.750 (1.79), 7.753 (1.74), 7.808 (0.44), 7.863 (5.62), 7.885 (4.90), 7.901 (3.87), 7.904 (3.78), 7.928 (0.65), 8.097 (4.54), 8.101 (5.12), 8.116 (4.18), 8.120 (4.40), 8.135 (0.51), 8.437 (5.84), 8.477 (5.73); LC-MS (method 7): Rₜ=1.21 min; MS (ESIpos): m/z=459 [M+H]⁺

### Example 75

### 6-(Azetidin-1-yl)-N-(3-ethoxybenzene-1-sulfonyl)-4-fluoro-1-benzofuran-2-carboxamide

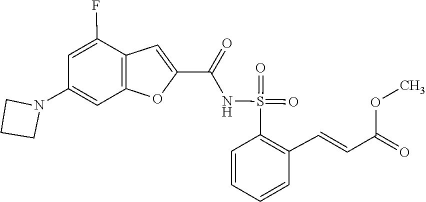 [SMILES: COC(=O)/C=C/c1ccccc1S(=O)(=O)NC(=O)c1cc2c(F)cc(N3CCC3)cc2o1]

Synthesized and purified analogously to Example 3 using commercially available 3-ethoxybenzene-1-sulfonamide (CAS: 1247894-17-2, 103 mg, 510 μmol) instead of 2-(cyclopentyloxy)-5-ethylbenzene-1-sulfonamide to give 30.0 mg (85% purity, 14% yield) of the title compound as a yellow solid. ¹H-NMR (400 MHz, DMSO-d₆) δ [ppm]: 1.310 (0.48), 1.330 (6.88), 1.347 (16.00), 1.365 (7.07), 1.747 (1.62), 1.754 (0.72), 1.764 (0.67), 2.291 (0.60), 2.308 (1.88), 2.318 (0.99), 2.327 (3.51), 2.336 (1.19), 2.346 (2.23), 2.364 (0.80), 2.518 (8.67), 2.523 (6.11), 2.669 (0.74), 2.673 (0.56), 3.081 (0.69), 3.087 (1.30), 3.098 (1.30), 3.103 (0.76), 3.877 (5.72), 3.885 (1.57), 3.896 (9.60), 3.914 (5.77), 4.032 (0.44), 4.049 (0.44), 4.075 (1.94), 4.092 (6.32), 4.110 (6.34), 4.127 (1.98), 6.311 (2.07), 6.316 (2.63), 6.340 (1.57), 6.345 (3.41), 6.349 (4.43), 6.352 (5.13), 6.355 (3.23), 7.260 (1.20), 7.267 (1.80), 7.275 (2.22), 7.280 (1.55), 7.282 (1.72), 7.289 (1.58), 7.452 (1.77), 7.455 (3.57), 7.458 (3.14), 7.462 (2.83), 7.524 (0.60), 7.536 (6.74), 7.538 (6.70), 7.543 (3.33), 7.547 (3.19), 7.552 (3.87), 7.571 (0.64), 7.875 (4.47); LC-MS (method 3): Rₜ=1.26 min; MS (ESIneg): m/z=417 [M−H]⁻

### Example 76

### 2-{[6-(Azetidin-1-yl)-4-fluoro-1-benzofuran-2-carbonyl]sulfamoyl}phenyl diethylcarbamate

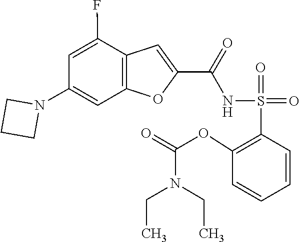 [SMILES: CCN(CC)C(=O)Oc1ccccc1S(=O)(=O)NC(=O)c1cc2c(F)cc(N3CCC3)cc2o1]

Synthesized and purified analogously to Example 3 using 2-sulfamoylphenyl diethylcarbamate (139 mg, 510 μmol) instead of 2-(cyclopentyloxy)-5-ethylbenzene-1-sulfonamide to give 35.0 mg (16% yield) of the title compound as a yellow solid. ¹H-NMR (400 MHz, DMSO-d₆) δ [ppm]: 1.014 (6.88), 1.032 (16.00), 1.050 (7.19), 1.106 (6.66), 1.124 (15.46), 1.142 (6.79), 2.288 (0.99), 2.306 (3.05), 2.325 (4.74), 2.337 (1.42), 2.344 (3.35), 2.361 (1.07), 2.518 (2.38), 2.523 (1.71), 2.665 (0.57), 2.669 (0.82), 2.673 (0.58), 3.185 (1.91), 3.203 (5.87), 3.221 (5.80), 3.238 (1.86), 3.414 (1.47), 3.465 (2.42), 3.483 (5.84), 3.501 (5.62), 3.519 (1.89), 3.872 (9.43), 3.891 (15.47), 3.909 (8.90), 6.306 (3.44), 6.310 (4.32), 6.337 (15.39), 7.271 (4.52), 7.274 (4.87), 7.291 (5.05), 7.294 (5.05), 7.428 (2.17), 7.430 (2.26), 7.447 (4.13), 7.466 (2.62), 7.469 (2.53), 7.689 (2.12), 7.694 (2.20), 7.710 (3.00), 7.712 (3.00), 7.728 (1.70), 7.732 (1.64), 7.879 (2.91), 7.987 (4.79), 7.991 (4.73), 8.007 (4.49), 8.011 (4.23); LC-MS (method 7): Rₜ=1.25 min; MS (ESIpos): m/z=490 [M+H]⁺

### Example 77

### 6-(Azetidin-1-yl)-N-[2-(ethanesulfonyl)benzene-1-sulfonyl]-4-fluoro-1-benzofuran-2-carboxamide

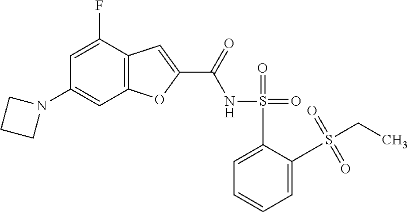 [SMILES: CCS(=O)(=O)c1ccccc1S(=O)(=O)NC(=O)c1cc2c(F)cc(N3CCC3)cc2o1]

Synthesized and purified analogously to Example 3 using commercially available 2-(ethanesulfonyl)benzene-1-sulfonamide (CAS: 147542-67-4, 127 mg, 510 μmol) instead of 2-(cyclopentyloxy)-5-ethylbenzene-1-sulfonamide to give 31.0 mg (15% yield) of the title compound as a yellow solid. ¹H-NMR (400 MHz, DMSO-d₆) δ [ppm]: 1.161 (7.06), 1.180 (16.00), 1.198 (7.53), 2.069 (0.83), 2.279 (0.87), 2.297 (2.91), 2.316 (4.37), 2.334 (3.28), 2.352 (1.07), 2.535 (0.76), 3.432 (0.88), 3.505 (1.21), 3.658 (2.33), 3.676 (6.92), 3.695 (6.86), 3.714 (2.28), 3.870 (7.86), 3.888 (13.64), 3.906 (7.73), 6.312 (3.46), 6.333 (7.86), 7.971 (6.09), 7.983 (3.31), 7.987 (3.29), 8.000 (4.81), 8.002 (4.88), 8.015 (3.22), 8.019 (3.05), 8.034 (1.21), 8.038 (0.88), 8.158 (3.64), 8.163 (3.34), 8.177 (2.55), 8.180 (2.87), 8.379 (3.48), 8.383 (3.00), 8.398 (3.11), 8.402 (3.00); LC-MS (method 3): Rₜ=1.23 min; MS (ESIneg): m/z=465 [M−H]⁻

### Example 78

### 6-(azetidin-1-yl)-N-(5-tert-butyl-2-ethoxybenzene-1-sulfonyl)-4-fluoro-1-benzofuran-2-carboxamide

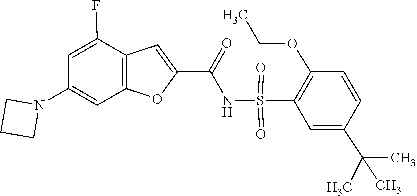 [SMILES: CCOc1ccc(C(C)(C)C)cc1S(=O)(=O)NC(=O)c1cc2c(F)cc(N3CCC3)cc2o1]

Synthesized and purified analogously to Example 3 using 5-tert-butyl-2-ethoxybenzene-1-sulfonamide (328 mg, 1.28 mmol) instead of 2-(cyclopentyloxy)-5-ethylbenzene-1-sulfonamide to give 107 mg (19% yield) of the title compound as a white solid. ¹H-NMR (400 MHz, DMSO-d₆) δ [ppm]: 1.211 (1.11), 1.229 (2.60), 1.246 (1.51), 1.298 (16.00), 2.322 (0.47), 3.871 (0.92), 3.889 (1.53), 3.908 (0.90), 4.120 (0.96), 4.138 (0.96), 6.317 (0.45), 6.338 (0.86), 6.343 (0.65), 7.130 (0.62), 7.152 (0.68), 7.662 (0.41), 7.847 (1.02), 7.853 (0.97), 8.042 (0.52); LC-MS (method 3): Rₜ=1.45 min; MS (ESIpos): m/z=475 [M+H]⁺

### Example 79

### 6-(Azetidin-1-yl)-4-fluoro-N-[3-(propan-2-yl)pyridine-2-sulfonyl]-1-benzofuran-2-carboxamide

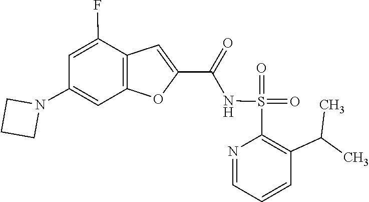 [SMILES: CC(C)c1cccnc1S(=O)(=O)NC(=O)c1cc2c(F)cc(N3CCC3)cc2o1]

Synthesized and purified analogously to Example 3 using 3-(propan-2-yl)pyridine-2-sulfonamide (100 mg, 499 μmol) instead of 2-(cyclopentyloxy)-5-ethylbenzene-1-sulfonamide to give 70.0 mg (38% yield) of the title compound as a yellow solid. ¹H-NMR (400 MHz, DMSO-d₆) δ [ppm]: 1.261 (15.88), 1.278 (16.00), 2.302 (0.46), 2.319 (1.50), 2.327 (0.93), 2.338 (2.06), 2.349 (0.62), 2.357 (1.55), 2.375 (0.50), 2.518 (1.70), 2.523 (1.08), 2.669 (0.40), 3.890 (4.46), 3.908 (7.40), 3.927 (4.62), 3.947 (0.99), 6.336 (1.69), 6.340 (1.86), 6.365 (1.48), 6.370 (2.09), 6.384 (2.98), 6.387 (3.49), 7.655 (1.58), 7.666 (1.63), 7.675 (1.72), 7.686 (1.74), 7.961 (3.09), 8.163 (1.88), 8.167 (1.95), 8.183 (1.77), 8.186 (1.76), 8.452 (2.66), 8.456 (2.71), 8.464 (2.61), 8.468 (2.38), LC-MS (method 3): Rₜ=1.29 min; MS (ESIpos): m/z=418 [M+H]⁺

### Example 80

### 6-(Azetidin-1-yl)-N-(2,2-dimethyl-3,4-dihydro-2H-1-benzopyran-8-sulfonyl)-4-fluoro-1-benzofuran-2-carboxamide

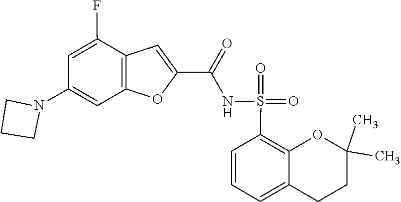 [SMILES: CC1(C)CCc2cccc(S(=O)(=O)NC(=O)c3cc4c(F)cc(N5CCC5)cc4o3)c2O1]

Synthesized and purified analogously to Example 3 using 2,2-dimethyl-3,4-dihydro-2H-1-benzopyran-8-sulfonamide (123 mg, 510 μmol) instead of 2-(cyclopentyloxy)-5-ethylbenzene-1-sulfonamide to give 51.0 mg (85% purity, 22% yield) as a yellow solid. ¹H-NMR (400 MHz, DMSO-d₆) δ [ppm]: 1.176 (16.00), 1.209 (1.62), 1.226 (1.57), 1.727 (0.90), 1.744 (1.96), 1.760 (0.97), 2.302 (1.03), 2.321 (1.58), 2.339 (1.17), 2.522 (0.50), 2.729 (0.82), 2.746 (1.58), 2.762 (0.78), 3.848 (0.48), 3.870 (2.80), 3.888 (4.40), 3.906 (2.44), 6.313 (0.93), 6.317 (1.08), 6.346 (1.44), 6.353 (2.25), 6.973 (0.88), 6.993 (1.68), 7.012 (0.97), 7.394 (0.87), 7.411 (0.80), 7.693 (1.01), 7.696 (1.01), 7.713 (0.98), 7.717 (0.92), 8.091 (1.16), 12.292 (1.11), LC-MS (method 6): Rₜ=1.34 min; MS (ESIpos): m/z=459 [M+H]⁺

### Example 81

### 6-(Azetidin-1-yl)-N-[2-(2,2-difluoroethoxy)-6-ethoxybenzene-1-sulfonyl]-4-fluoro-1-benzofuran-2-carboxamide

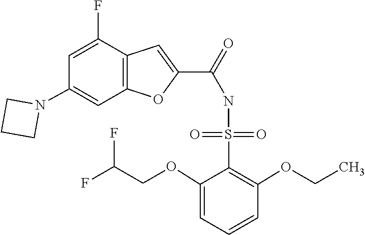

Synthesized and purified analogously to Example 3 using 2-(2,2-difluoroethoxy)-6-ethoxybenzene-1-sulfonamide (150 mg, 533 μmol) instead of 2-(cyclopentyloxy)-5-ethylbenzene-1-sulfonamide to give 15.0 mg (90% purity, 6% yield) of the title compound as an offwhite solid. ¹H-NMR (400 MHz, DMSO-d₆) δ [ppm]: 1.163 (7.02), 1.171 (1.09), 1.180 (16.00), 1.188 (1.89), 1.192 (1.59), 1.198 (7.32), 1.205 (0.86), 1.209 (1.06), 2.074 (1.29), 2.294 (0.69), 2.313 (2.15), 2.322 (1.52), 2.332 (3.72), 2.349 (2.35), 2.368 (0.74), 2.518 (3.99), 2.523 (2.49), 2.664 (0.71), 2.669 (1.01), 2.673 (0.72), 3.880 (6.35), 3.899 (10.57), 3.917 (6.03), 4.063 (1.62), 4.080 (5.19), 4.098 (5.31), 4.116 (1.68), 4.354 (1.75), 4.364 (1.85), 4.390 (3.69), 4.399 (3.69), 4.425 (1.78), 4.435 (1.61), 6.191 (0.55), 6.201 (1.18), 6.211 (0.53), 6.320 (2.29), 6.324 (2.73), 6.328 (1.71), 6.338 (2.66), 6.349 (2.35), 6.354 (3.07), 6.362 (4.50), 6.364 (5.06), 6.465 (0.46), 6.474 (1.04), 6.484 (0.53), 6.852 (3.33), 6.859 (3.03), 6.873 (3.44), 6.880 (3.25), 7.499 (1.64), 7.520 (2.98), 7.542 (1.50), 7.991 (2.29), 12.202 (2.29), LC-MS (method 3): Rₜ=1.27 min; MS (ESIpos): m/z=499 [M+H]⁺

### Example 82

### 6-(Azetidin-1-yl)-N-[5-bromo-2-(trifluoromethoxy)benzene-1-sulfonyl]-4-fluoro-1-benzofuran-2-carboxamide

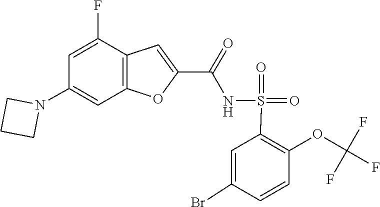 [SMILES: O=C(NS(=O)(=O)c1cc(Br)ccc1OC(F)(F)F)c1cc2c(F)cc(N3CCC3)cc2o1]

Synthesized and purified analogously to Example 3 using 5-bromo-2-(trifluoromethoxy)benzene-1-sulfonamide (158 mg, 493 μmol) instead of 2-(cyclopentyloxy)-5-ethylbenzene-1-sulfonamide to give 123 mg (52% yield) of the title compound as a yellow solid. ¹H-NMR (400 MHz, DMSO-d₆) δ [ppm]: 2.074 (16.00), 2.307 (1.03), 2.326 (1.68), 2.336 (0.49), 2.344 (1.12), 2.518 (1.10), 2.523 (0.68), 3.876 (3.21), 3.894 (5.21), 3.913 (3.06), 6.314 (1.17), 6.318 (1.44), 6.346 (5.64), 7.818 (1.32), 7.822 (1.84), 7.826 (1.32), 7.876 (1.62), 7.880 (1.40), 7.898 (3.00), 7.902 (2.63), 8.047 (3.81), 8.069 (2.80), LC-MS (method 3): Rₜ=1.88 min; MS (ESIpos): m/z=538 [M+H]⁺

### Example 83

### 6-(Azetidin-1-yl)-N-(2-tert-butoxy-6-propoxybenzene-1-sulfonyl)-4-fluoro-1-benzofuran-2-carboxamide

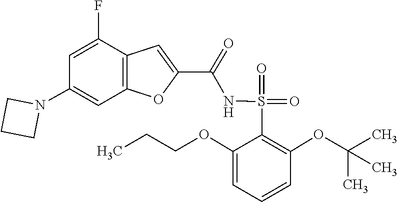 [SMILES: CCCOc1cccc(OC(C)(C)C)c1S(=O)(=O)NC(=O)c1cc2c(F)cc(N3CCC3)cc2o1]

Synthesized and purified analogously to Example 3 using 2-tert-butoxy-6-propoxybenzene-1-sulfonamide (150 mg, 522 μmol) instead of 2-(cyclopentyloxy)-5-ethylbenzene-1-sulfonamide to give 24.0 mg (10% yield) of the title compound as a light green solid. ¹H-NMR (400 MHz, DMSO-d₆) δ [ppm]: 0.817 (1.41), 0.836 (3.40), 0.854 (1.58), 1.421 (16.00), 1.577 (0.47), 1.594 (0.86), 1.613 (0.84), 1.630 (0.40), 2.313 (0.41), 2.332 (0.74), 2.350 (0.45), 2.518 (1.10), 2.522 (0.68), 3.880 (1.19), 3.899 (1.99), 3.917 (1.29), 3.938 (1.26), 3.955 (0.60), 6.323 (0.44), 6.352 (0.48), 6.368 (1.02), 6.791 (0.50), 6.812 (0.53), 6.855 (0.51), 6.875 (0.54), 7.418 (0.59), 7.989 (0.87), 11.906 (0.92). LC-MS (method 3): Rₜ=1.49 min; MS (ESIneg): m/z=503 [M−H]⁻

### Example 84

### 6-(Azetidin-1-yl)-N-{2-ethyl-6-[(propan-2-yl)oxy]benzene-1-sulfonyl}-4-fluoro-1-benzofuran-2-carboxamide

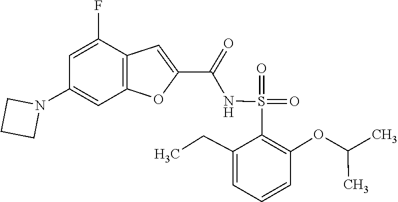 [SMILES: CCc1cccc(OC(C)C)c1S(=O)(=O)NC(=O)c1cc2c(F)cc(N3CCC3)cc2o1]

Synthesized and purified analogously to Example 3 using 2-ethyl-6-[(propan-2-yl)oxy]benzene-1-sulfonamide (150 mg, 616 μmol) instead of 2-(cyclopentyloxy)-5-ethylbenzene-1-sulfonamide to give 12.0 mg (5% yield) of the title compound as a brownish solid. ¹H-NMR (400 MHz, DMSO-d₆) δ [ppm]: 1.166 (15.82), 1.181 (16.00), 1.213 (3.10), 1.232 (7.09), 1.250 (3.05), 2.074 (0.86), 2.306 (1.15), 2.326 (2.06), 2.336 (0.79), 2.344 (1.27), 2.518 (1.97), 2.523 (1.21), 2.669 (0.53), 3.070 (0.77), 3.089 (2.56), 3.107 (2.51), 3.126 (0.75), 3.876 (3.45), 3.894 (5.63), 3.912 (3.23), 4.741 (0.64), 4.756 (0.86), 4.771 (0.64), 6.318 (1.05), 6.321 (1.33), 6.344 (3.58), 6.346 (3.77), 6.922 (1.34), 6.941 (1.43), 7.048 (1.07), 7.069 (1.18), 7.447 (0.82), 7.467 (1.30), 7.487 (0.69), 8.040 (1.97), 12.067 (2.00), LC-MS (method 3): Rₜ=1.46 min; MS (ESIneg): m/z=459 [M−H]⁻

### Example 85

### 6-(Azetidin-1-yl)-N-[2-(benzyloxy)-6-ethylbenzene-1-sulfonyl]-4-fluoro-1-benzofuran-2-carboxamide

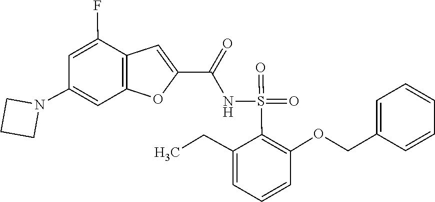 [SMILES: CCc1cccc(OCc2ccccc2)c1S(=O)(=O)NC(=O)c1cc2c(F)cc(N3CCC3)cc2o1]

Synthesized and purified analogously to Example 3 using 2-(benzyloxy)-6-ethylbenzene-1-sulfonamide (150 mg, 515 μmol) instead of 2-(cyclopentyloxy)-5-ethylbenzene-1-sulfonamide to give 83.0 mg (36% yield) of the title compound as a yellow solid. ¹H-NMR (400 MHz, DMSO-d₆) δ [ppm]: 1.216 (6.42), 1.234 (16.00), 1.253 (6.63), 2.300 (0.74), 2.318 (2.75), 2.326 (2.39), 2.336 (3.70), 2.355 (2.47), 2.373 (0.77), 2.518 (6.57), 2.522 (3.98), 2.660 (0.49), 2.664 (1.13), 2.669 (1.57), 2.673 (1.13), 2.678 (0.49), 3.094 (1.54), 3.112 (4.93), 3.131 (4.83), 3.149 (1.44), 3.889 (6.65), 3.908 (11.07), 3.925 (6.34), 5.308 (8.60), 6.330 (5.98), 6.332 (6.52), 6.353 (2.85), 6.357 (2.18), 6.933 (3.11), 6.953 (3.24), 6.982 (2.88), 7.001 (3.31), 7.093 (1.44), 7.096 (2.44), 7.100 (1.03), 7.114 (6.34), 7.117 (3.62), 7.132 (5.21), 7.157 (1.46), 7.161 (3.00), 7.164 (1.87), 7.173 (0.95), 7.179 (3.44), 7.186 (0.72), 7.197 (0.95), 7.335 (4.21), 7.352 (3.75), 7.393 (2.41), 7.412 (3.42), 7.433 (1.85), 7.933 (2.54), 12.373 (1.95), LC-MS (method 3): Rₜ=1.48 min; MS (ESIpos): m/z=509 [M+H]⁺

### Example 86

### 6-(Azetidin-1-yl)-4-fluoro-N-{2-methyl-6-[(propan-2-yl)oxy]benzene-1-sulfonyl}-1-benzofuran-2-carboxamide

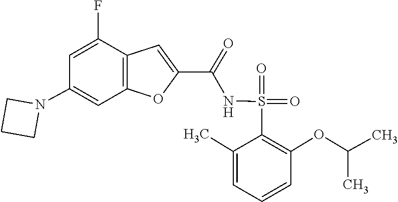 [SMILES: Cc1cccc(OC(C)C)c1S(=O)(=O)NC(=O)c1cc2c(F)cc(N3CCC3)cc2o1]

Synthesized and purified analogously to Example 3 using 2-methyl-6-[(propan-2-yl)oxy]benzene-1-sulfonamide (117 mg, 510 μmol) instead of 2-(cyclopentyloxy)-5-ethylbenzene-1-sulfonamide to give 53.0 mg (2% yield) of the title compound as a yellow solid. ¹H-NMR (400 MHz, DMSO-d₆) δ [ppm]: 1.175 (15.88), 1.190 (16.00), 2.074 (1.73), 2.306 (1.08), 2.326 (1.94), 2.336 (0.63), 2.343 (1.17), 2.518 (1.93), 2.522 (1.24), 2.621 (10.94), 2.664 (0.44), 2.668 (0.60), 2.673 (0.42), 3.875 (3.32), 3.893 (5.38), 3.912 (3.10), 4.748 (0.74), 4.763 (1.01), 4.779 (0.74), 6.316 (1.16), 6.320 (1.43), 6.347 (5.22), 6.899 (1.38), 6.918 (1.46), 7.064 (1.16), 7.085 (1.30), 7.414 (1.08), 7.434 (1.49), 7.454 (0.88), 8.031 (2.07), 12.140 (1.64), LC-MS (method 6): Rₜ=1.42 min; MS (ESIpos): m/z=447 [M+H]⁺

### Example 87

### 6-(azetidin-1-yl)-4-fluoro-N-(4-methylnaphthalene-1-sulfonyl)-1-benzofuran-2-carboxamide

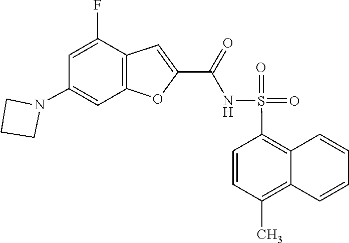 [SMILES: Cc1ccc(S(=O)(=O)NC(=O)c2cc3c(F)cc(N4CCC4)cc3o2)c2ccccc12]

Synthesized and purified analogously to Example 3 using 4-methylnaphthalene-1-sulfonamide (113 mg, 510 μmol) instead of 2-(cyclopentyloxy)-5-ethylbenzene-1-sulfonamide to give 36.0 mg (18% yield) of the title compound as a yellow solid. ¹H-NMR (400 MHz, DMSO-d₆) δ [ppm]: 2.074 (1.14), 2.271 (0.61), 2.288 (1.89), 2.307 (2.73), 2.326 (3.28), 2.331 (1.44), 2.336 (0.71), 2.344 (0.69), 2.518 (4.57), 2.522 (2.92), 2.664 (0.90), 2.668 (1.24), 2.673 (0.87), 2.754 (16.00), 3.851 (5.84), 3.869 (9.53), 3.887 (5.54), 6.287 (1.86), 6.290 (2.79), 6.300 (3.74), 6.303 (4.63), 6.316 (2.76), 6.321 (1.75), 7.590 (2.27), 7.610 (2.44), 7.667 (0.89), 7.670 (0.96), 7.684 (1.70), 7.687 (2.15), 7.705 (1.73), 7.708 (1.59), 7.724 (1.41), 7.728 (1.51), 7.746 (1.90), 7.749 (1.58), 7.763 (0.89), 7.882 (1.21), 8.184 (2.25), 8.203 (2.00), LC-MS (method 3): Rₜ=1.46 min; MS (ESIpos): m/z=439 [M+H]⁺

### Example 88

### 6-(Azetidin-1-yl)-N-(2,6-difluoro-3-methylbenzene-1-sulfonyl)-4-fluoro-1-benzofuran-2-carboxamide

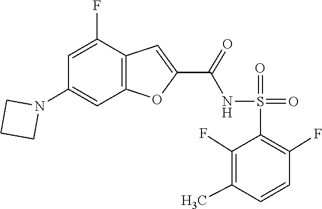 [SMILES: Cc1ccc(F)c(S(=O)(=O)NC(=O)c2cc3c(F)cc(N4CCC4)cc3o2)c1F]

Synthesized and purified analogously to Example 3 using 2,6-difluoro-3-methylbenzene-1-sulfonamide (106 mg, 510 μmol) instead of 2-(cyclopentyloxy)-5-ethylbenzene-1-sulfonamide to give 103 mg (54% yield) of the title compound as a yellow solid. ¹H-NMR (400 MHz, DMSO-d₆) δ [ppm]: 2.074 (0.65), 2.240 (13.53), 2.294 (1.00), 2.311 (3.08), 2.322 (1.77), 2.331 (5.07), 2.349 (3.36), 2.367 (1.09), 2.518 (3.75), 2.523 (2.34), 2.665 (0.70), 2.669 (1.02), 2.673 (0.72), 3.483 (0.72), 3.901 (15.73), 3.920 (9.20), 6.321 (3.48), 6.325 (4.26), 6.354 (16.00), 7.202 (1.47), 7.226 (2.79), 7.249 (1.62), 7.630 (0.84), 7.651 (1.77), 7.666 (1.76), 7.687 (0.82), 7.915 (3.66), LC-MS (method 6): Rₜ=1.19 min; MS (ESIneg): m/z=423 [M−H]⁻

### Example 89

### 6-(Azetidin-1-yl)-4-fluoro-N-(5-propanamidonaphthalene-1-sulfonyl)-1-benzofuran-2-carboxamide

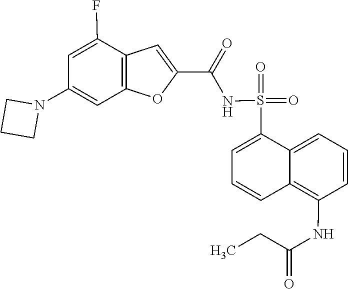 [SMILES: CCC(=O)Nc1cccc2c(S(=O)(=O)NC(=O)c3cc4c(F)cc(N5CCC5)cc4o3)cccc12]

Synthesized and purified analogously to Example 3 using N-(5-sulfamoylnaphthalen-1-yl)propanamide (75.0 mg, 269 μmol) instead of 2-(cyclopentyloxy)-5-ethylbenzene-1-sulfonamide to give 40.0 mg (34% yield) of the title compound as a yellow solid. ¹H-NMR (400 MHz, DMSO-d₆) δ [ppm]: 1.128 (5.72), 1.147 (11.90), 1.166 (6.05), 2.075 (4.53), 2.272 (1.03), 2.290 (3.17), 2.309 (4.52), 2.318 (1.70), 2.327 (4.90), 2.345 (1.20), 2.466 (1.56), 2.518 (5.96), 2.523 (4.58), 2.539 (1.03), 2.660 (0.45), 2.665 (0.97), 2.669 (1.40), 2.673 (0.98), 2.678 (0.43), 3.855 (9.73), 3.873 (16.00), 3.891 (9.40), 6.290 (3.06), 6.294 (5.55), 6.301 (7.07), 6.303 (8.43), 6.320 (4.89), 6.324 (3.36), 7.687 (2.12), 7.706 (5.32), 7.727 (6.00), 7.736 (5.68), 7.740 (5.38), 7.751 (2.09), 7.760 (6.09), 7.780 (3.92), 7.889 (3.45), 8.399 (12.04), 8.418 (8.65), 8.525 (3.26), 8.546 (2.96), 10.034 (4.15), LC-MS (method 1): Rₜ=1.13 min; MS (ESIpos): m/z=496 [M+H]⁺

### Example 90

### Ethyl 8-{[6-(azetidin-1-yl)-4-fluoro-1-benzofuran-2-carbonyl]sulfamoyl}imidazo[1,2-a]pyridine-2-carboxylate

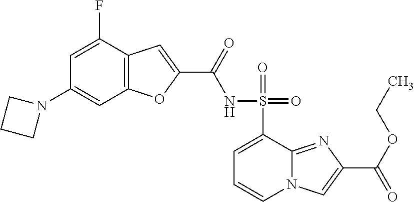 [SMILES: CCOC(=O)c1cn2cccc(S(=O)(=O)NC(=O)c3cc4c(F)cc(N5CCC5)cc4o3)c2n1]

Synthesized and purified analogously to Example 3 using ethyl 8-sulfamoylimidazo[1,2-a]pyridine-2-carboxylate (75.0 mg, 279 μmol) instead of 2-(cyclopentyloxy)-5-ethylbenzene-1-sulfonamide to give 25.0 mg (95% purity, 21% yield) of the title compound as a yellow solid. ¹H-NMR (400 MHz, DMSO-d6) δ[ppm]: 1.262 (7.04), 1.280 (16.00), 1.297 (7.12), 2.276 (0.50), 2.294 (1.53), 2.313 (2.19), 2.323 (1.08), 2.332 (2.16), 2.349 (0.55), 2.518 (2.24), 2.523 (1.54), 2.539 (0.63), 2.665 (0.50), 2.669 (0.70), 2.673 (0.50), 3.859 (5.10), 3.877 (8.04), 3.895 (4.79), 4.269 (2.07), 4.287 (6.78), 4.304 (6.67), 4.322 (1.96), 6.302 (1.50), 6.306 (2.57), 6.316 (3.89), 6.332 (2.33), 6.336 (1.60), 7.233 (2.74), 7.250 (4.19), 7.268 (2.62), 8.073 (2.80), 8.121 (2.53), 8.124 (2.85), 8.139 (2.59), 8.142 (2.47), 8.720 (12.69), 8.853 (2.68), 8.856 (2.68), 8.870 (2.70), 8.873 (2.52), LC-MS (method 1): Rₜ=1.16 min; MS (ESIpos): m/z=487 [M+H]⁺

### Example 91

### 6-(Azetidin-1-yl)-4-fluoro-N-[3-methyl-1-(propan-2-yl)-1H-pyrazole-4-sulfonyl]-1-benzofuran-2-carboxamide

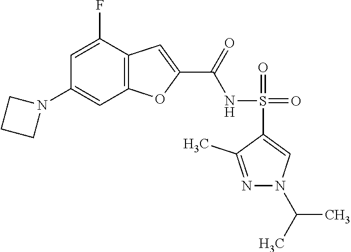 [SMILES: Cc1nn(C(C)C)cc1S(=O)(=O)NC(=O)c1cc2c(F)cc(N3CCC3)cc2o1]

Synthesized and purified analogously to Example 3 using 3-methyl-1-(propan-2-yl)-1H-pyrazole-4-sulfonamide (104 mg, 510 μmol) instead of 2-(cyclopentyloxy)-5-ethylbenzene-1-sulfonamide to give 67.0 mg (95% purity, 36% yield) of the title compound as a yellow solid. ¹H-NMR (400 MHz, DMSO-d₆) δ [ppm]: 1.383 (16.00), 1.400 (15.96), 2.310 (1.05), 2.329 (1.66), 2.345 (15.90), 2.365 (0.42), 2.518 (0.93), 2.523 (0.60), 3.877 (3.18), 3.896 (5.20), 3.914 (3.02), 4.504 (1.04), 4.520 (1.37), 4.537 (1.00), 6.311 (1.24), 6.314 (1.48), 6.340 (1.05), 6.344 (1.75), 6.352 (2.35), 6.354 (2.68), 6.358 (1.63), 7.913 (2.97), 8.390 (4.97), LC-MS (method 3): Rₜ=1.20 min; MS (ESIpos): m/z=421 [M+H]⁺

### Example 92

### 6-(Azetidin-1-yl)-N-{2-[(2,2-difluoroethyl)amino]-5-(trifluoromethyl)benzene-1-sulfonyl}-4-fluoro-1-benzofuran-2-carboxamide

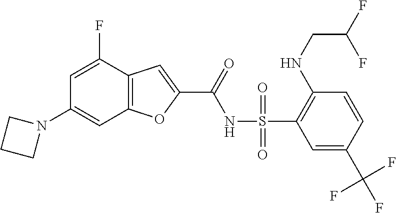 [SMILES: O=C(NS(=O)(=O)c1cc(C(F)(F)F)ccc1NCC(F)F)c1cc2c(F)cc(N3CCC3)cc2o1]

Synthesized and purified analogously to Example 3 using 2-[(2,2-difluoroethyl)amino]-5-(trifluoromethyl)benzene-1-sulfonamide (155 mg, 510 μmol) instead of 2-(cyclopentyloxy)-5-ethylbenzene-1-sulfonamide to give 60.0 mg (26% yield) of the title compound as a yellow solid. ¹H-NMR (400 MHz, DMSO-d₆) δ [ppm]: 2.075 (4.65), 2.294 (0.98), 2.312 (3.06), 2.323 (2.32), 2.331 (5.25), 2.349 (3.31), 2.367 (1.04), 2.518 (4.52), 2.523 (3.12), 2.660 (0.47), 2.665 (1.07), 2.669 (1.50), 2.673 (1.08), 2.678 (0.45), 3.478 (1.13), 3.813 (1.36), 3.880 (10.01), 3.899 (16.00), 3.917 (9.08), 6.053 (0.67), 6.062 (1.43), 6.071 (0.62), 6.191 (1.28), 6.200 (2.92), 6.209 (1.32), 6.314 (3.10), 6.318 (4.23), 6.338 (9.19), 6.344 (5.79), 6.737 (1.45), 7.180 (3.17), 7.202 (3.39), 7.735 (2.86), 7.740 (3.40), 7.756 (4.16), 7.762 (3.21), 8.002 (5.27), 8.006 (5.14), LC-MS (method 1): Rₜ=1.36 min; MS (ESIpos): m/z=522 [M+H]⁺

### Example 93

### [3-sulfamoyl-4-(trifluoromethoxy)phenyl]methyl 6-(azetidin-1-yl)-4-fluoro-1-benzofuran-2-carboxylate

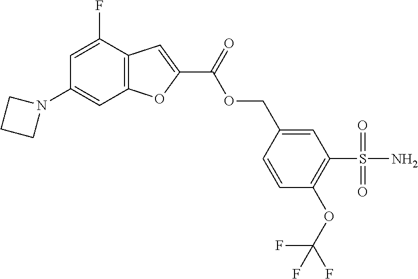 [SMILES: NS(=O)(=O)c1cc(COC(=O)c2cc3c(F)cc(N4CCC4)cc3o2)ccc1OC(F)(F)F]

Synthesized and purified analogously to Example 3 using 5-(hydroxymethyl)-2-(trifluoromethoxy)benzene-1-sulfonamide (75.0 mg, 277 μmol) instead of 2-(cyclopentyloxy)-5-ethylbenzene-1-sulfonamide to give 5.00 mg (4% yield) of the title compound as an offwhite solid. ¹H-NMR (400 MHz, DMSO-d₆) δ [ppm]: 2.301 (0.42), 2.318 (1.64), 2.322 (1.69), 2.327 (1.82), 2.331 (1.58), 2.337 (2.34), 2.356 (1.49), 2.373 (0.53), 2.518 (16.00), 2.523 (11.36), 2.659 (0.44), 2.665 (0.98), 2.669 (1.40), 2.673 (1.05), 2.678 (0.54), 3.884 (3.94), 3.902 (6.52), 3.920 (3.99), 5.437 (6.31), 6.329 (1.46), 6.333 (1.62), 6.359 (1.48), 6.362 (1.69), 6.439 (2.08), 6.442 (2.71), 6.445 (2.17), 7.591 (1.06), 7.595 (1.07), 7.612 (1.33), 7.616 (1.27), 7.710 (5.00), 7.751 (4.59), 7.753 (4.91), 7.818 (1.42), 7.823 (1.53), 7.838 (1.21), 7.844 (1.33), 8.029 (2.70), 8.035 (2.64), LC-MS (method 1): Rₜ=1.29 min; MS (ESIpos): m/z=489 [M+H]⁺

### Example 94

### 6-(Azetidin-1-yl)-4-fluoro-N-(2-methylpyridine-3-sulfonyl)-1-benzofuran-2-carboxamide

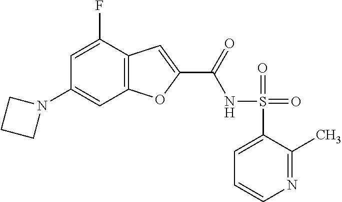 [SMILES: Cc1ncccc1S(=O)(=O)NC(=O)c1cc2c(F)cc(N3CCC3)cc2o1]

Synthesized and purified analogously to Example 3 using 2-methylpyridine-3-sulfonamide (123 mg, 714 μmol) instead of 2-(cyclopentyloxy)-5-ethylbenzene-1-sulfonamide to give 140 mg (57% yield) of the title compound as a yellow solid. ¹H-NMR (400 MHz, DMSO-d₆) δ [ppm]: 2.287 (0.49), 2.305 (1.55), 2.323 (2.48), 2.342 (1.74), 2.360 (0.59), 2.522 (2.30), 2.669 (0.43), 2.810 (16.00), 3.872 (4.51), 3.890 (7.56), 3.908 (4.45), 6.306 (1.61), 6.310 (1.88), 6.341 (4.36), 6.343 (4.20), 7.548 (1.07), 7.560 (1.18), 7.568 (1.22), 7.580 (1.16), 7.855 (1.14), 8.389 (1.45), 8.393 (1.49), 8.410 (1.44), 8.413 (1.39), 8.712 (2.03), 8.715 (2.03), 8.724 (2.10), 8.728 (1.89), LC-MS (method 1): Rₜ=0.93 min; MS (ESIpos): m/z=390 [M+H]⁺

### Example 95

### 2-{[6-(Azetidin-1-yl)-4-fluoro-1-benzofuran-2-carbonyl]sulfamoyl}-N,N-dimethylpyridine-3-carboxamide

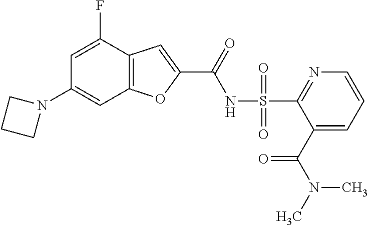 [SMILES: CN(C)C(=O)c1cccnc1S(=O)(=O)NC(=O)c1cc2c(F)cc(N3CCC3)cc2o1]

Synthesized and purified analogously to Example 3 using N,N-dimethyl-2-sulfamoylpyridine-3-carboxamide (117 mg, 510 μmol) instead of 2-(cyclopentyloxy)-5-ethylbenzene-1-sulfonamide to give 20.0 mg (9% yield) of the title compound as a yellow solid. ¹H-NMR (400 MHz, DMSO-d₆) δ [ppm]: 2.074 (3.66), 2.315 (1.02), 2.322 (0.56), 2.333 (1.50), 2.352 (1.10), 2.518 (0.80), 2.522 (0.52), 2.879 (14.97), 2.992 (16.00), 3.905 (5.07), 3.923 (2.96), 6.335 (5.21), 6.364 (1.41), 6.368 (1.19), 6.575 (0.49), 7.741 (1.70), 7.752 (1.76), 7.760 (1.79), 7.772 (1.94), 7.960 (2.91), 8.007 (1.87), 8.011 (2.09), 8.026 (1.66), 8.031 (1.64), 8.697 (2.12), 8.701 (2.00), 8.708 (1.93), 8.713 (1.87), LC-MS (method 3): Rₜ=0.97 min; MS (ESIpos): m/z=447 [M+H]⁺

### Example 96

### 6-(Azetidin-1-yl)-N-[2-(5,6-dihydro-1,4,2-dioxazin-3-yl)benzene-1-sulfonyl]-4-fluoro-1-benzofuran-2-carboxamide

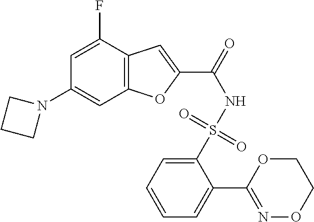 [SMILES: O=C(NS(=O)(=O)c1ccccc1C1=NOCCO1)c1cc2c(F)cc(N3CCC3)cc2o1]

Synthesized and purified analogously to Example 3 using 2-(5,6-dihydro-1,4,2-dioxazin-3-yl)benzene-1-sulfonamide (75.0 mg, 310 μmol) instead of 2-(cyclopentyloxy)-5-ethylbenzene-1-sulfonamide to give 35.0 mg (95% purity, 28% yield) of the title compound as an offwhite solid. ¹H-NMR (400 MHz, DMSO-d₆) δ [ppm]: 1.169 (0.49), 1.210 (1.44), 1.227 (1.43), 2.074 (6.22), 2.300 (1.04), 2.318 (3.89), 2.322 (3.19), 2.327 (3.48), 2.337 (5.32), 2.355 (3.36), 2.373 (1.11), 2.518 (7.78), 2.523 (5.80), 2.539 (0.72), 2.660 (0.78), 2.665 (1.77), 2.669 (2.55), 2.673 (1.79), 2.678 (0.75), 3.850 (0.47), 3.877 (10.03), 3.896 (16.00), 3.913 (9.61), 4.420 (2.80), 4.430 (3.91), 4.441 (3.50), 4.580 (4.24), 4.586 (4.12), 4.592 (4.31), 4.602 (3.30), 6.310 (3.84), 6.314 (3.96), 6.339 (3.62), 6.344 (4.12), 6.396 (4.92), 7.632 (6.20), 7.730 (0.86), 7.745 (2.43), 7.748 (2.52), 7.766 (4.39), 7.784 (2.84), 7.787 (3.15), 7.806 (2.96), 7.827 (1.41), 8.024 (2.55), 8.043 (2.19), LC-MS (method 1): Rₜ=1.28 min; MS (ESIpos): m/z=460 [M+H]⁺

### Example 97

### 6-(Azetidin-1-yl)-N-[2-chloro-5-(dimethylamino)benzene-1-sulfonyl]-4-fluoro-1-benzofuran-2-carboxamide

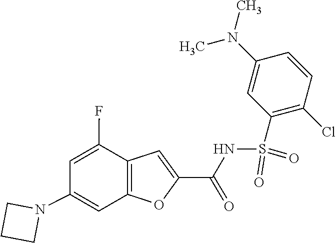 [SMILES: CN(C)c1ccc(Cl)c(S(=O)(=O)NC(=O)c2cc3c(F)cc(N4CCC4)cc3o2)c1]

Synthesized and purified analogously to Example 3 using 2-chloro-5-(dimethylamino)benzene-1-sulfonamide (120 mg, 510 μmol) instead of 2-(cyclopentyloxy)-5-ethylbenzene-1-sulfonamide to give 25.0 mg (85% purity, 11% yield) of the title compound as yellow solid. ¹H-NMR (400 MHz, DMSO-d₆) δ [ppm]: 2.328 (0.58), 2.347 (0.73), 2.366 (0.56), 2.522 (0.95), 2.844 (16.00), 3.892 (1.46), 3.911 (2.51), 3.929 (1.51), 6.321 (0.61), 6.335 (0.54), 6.339 (0.63), 6.364 (0.53), 6.368 (0.64), 6.407 (1.17), 6.410 (1.00), 7.677 (1.25), 7.699 (1.36), 7.757 (1.73), 7.759 (1.76), 8.111 (0.68), 8.118 (0.71), 8.133 (0.62), 8.139 (0.66), 8.424 (1.25), 8.430 (1.28), 10.712 (1.14), LC-MS (method 3): Rₜ=1.30 min; MS (ESIneg): m/z=450 [M−H]⁻

### Example 98

### 4-Sulfamoylnaphthalen-2-yl 6-(azetidin-1-yl)-4-fluoro-1-benzofuran-2-carboxylate

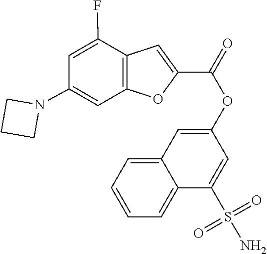 [SMILES: NS(=O)(=O)c1cc(OC(=O)c2cc3c(F)cc(N4CCC4)cc3o2)cc2ccccc12]

Synthesized and purified analogously to Example 3 using 3-hydroxynaphthalene-1-sulfonamide (75.0 mg, 336 μmol) instead of 2-(cyclopentyloxy)-5-ethylbenzene-1-sulfonamide to give 25.0 mg (19% yield) of the title compound as a yellow solid. ¹H-NMR (400 MHz, DMSO-d₆) δ [ppm]: 2.074 (0.98), 2.323 (1.03), 2.327 (2.18), 2.331 (1.38), 2.337 (0.98), 2.345 (3.20), 2.364 (4.66), 2.383 (3.71), 2.401 (1.37), 2.518 (14.87), 2.522 (10.76), 2.539 (7.52), 2.659 (0.41), 2.665 (0.84), 2.669 (1.21), 2.673 (0.93), 2.678 (0.48), 3.929 (9.69), 3.947 (16.00), 3.965 (9.94), 6.391 (3.70), 6.395 (4.22), 6.420 (3.81), 6.424 (4.26), 6.509 (5.42), 6.512 (7.23), 6.516 (5.82), 7.695 (0.69), 7.699 (1.24), 7.712 (3.34), 7.719 (5.10), 7.728 (8.11), 7.736 (4.64), 7.740 (3.87), 7.744 (4.25), 7.757 (1.72), 7.761 (1.12), 7.840 (11.99), 8.045 (11.96), 8.047 (13.06), 8.053 (10.04), 8.059 (10.94), 8.094 (0.52), 8.104 (3.21), 8.111 (2.96), 8.119 (1.86), 8.125 (2.53), 8.128 (3.09), 8.188 (6.05), 8.194 (6.05), 8.646 (3.05), 8.648 (3.09), 8.654 (1.62), 8.656 (1.43), 8.665 (2.64), 8.671 (2.91); LC-MS (method 1): Rₜ=1.34 min; MS (ESIpos): m/z=441 [M+H]⁺

### Example 99

### N-(5-Acetyl-2-chlorobenzene-1-sulfonyl)-6-(azetidin-1-yl)-4-fluoro-1-benzofuran-2-carboxamide

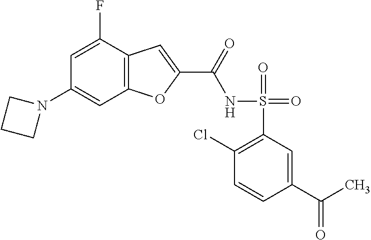 [SMILES: CC(=O)c1ccc(Cl)c(S(=O)(=O)NC(=O)c2cc3c(F)cc(N4CCC4)cc3o2)c1]

To a stirring solution of 6-(azetidin-1-yl)-4-fluoro-1-benzofuran-2-carboxylic acid (Intermediate 3, 77.4 mg, 329 μmol) and commercially available 5-acetyl-2-chlorobenzene-1-sulfonamide (CAS: 2350-44-9, 92.3 mg, 395 μmol) in DMF (1.5 mL) was added PyBOP (206 mg, 395 μmol; CAS-RN:[128625-52-5]) and N,N-diisopropylethylamine (230 μL, 1.3 mmol; CAS-RN:[7087-68-5]) subsequently. The reaction mixture was stirred at room temperature for 1 h. After completion the solution was filtrated and purified by preparative HPLC (water+0.1% HCOOH/acetonitrile gradient) to give 88.0 mg (56% yield) of the title compound as a yellow solid. ¹H-NMR (400 MHz, DMSO-d₆) δ [ppm]: 2.084 (16.00), 2.306 (0.77), 2.325 (1.26), 2.343 (0.84), 2.518 (1.26), 2.522 (0.80), 2.660 (10.44), 3.874 (2.34), 3.893 (3.72), 3.911 (2.07), 6.313 (0.77), 6.316 (0.96), 6.339 (2.10), 6.340 (2.13), 7.829 (1.30), 7.849 (1.41), 7.926 (0.56), 8.228 (0.79), 8.233 (0.81), 8.249 (0.71), 8.254 (0.73), 8.584 (2.08), 8.589 (2.01); LC-MS (method 2): Rₜ=0.78 min; MS (ESIneg): m/z=449 [M−H]⁻

### Example 100

### 6-(Azetidin-1-yl)-N-[2-chloro-5-(1-hydroxyethyl)benzene-1-sulfonyl]-4-fluoro-1-benzofuran-2-carboxamide (rac)

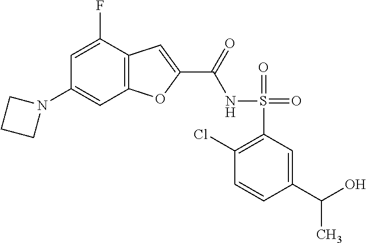 [SMILES: CC(O)c1ccc(Cl)c(S(=O)(=O)NC(=O)c2cc3c(F)cc(N4CCC4)cc3o2)c1]

Synthesized and purified analogously to

Example 99 using commercially available 2-chloro-5-(1-hydroxyethyl)benzene-1-sulfonamide (CAS: 2350-44-9, 93.1 mg, 395 μmol) instead of 5-acetyl-2-chlorobenzene-1-sulfonamide to give 24.0 mg (15% yield) of the title compound as a yellow solid. ¹H-NMR (400 MHz, DMSO-d₆) δ [ppm]: −0.002 (0.50), 1.335 (3.67), 1.352 (3.70), 2.084 (16.00), 2.307 (0.70), 2.326 (1.58), 2.344 (0.76), 2.518 (2.43), 2.523 (1.54), 2.665 (0.45), 2.669 (0.62), 2.673 (0.45), 3.875 (2.05), 3.893 (3.27), 3.912 (1.78), 4.831 (0.68), 4.848 (0.67), 6.313 (0.67), 6.316 (0.75), 6.344 (3.28), 7.591 (0.93), 7.611 (0.66), 8.142 (1.16), 8.147 (1.09); LC-MS (method 1): Rₜ=1.16 min; MS (ESIpos): m/z=453 [M+H]⁺

### Example 101

### Propan-2-yl 2-{[6-(azetidin-1-yl)-4-fluoro-1-benzofuran-2-carbonyl]sulfamoyl}benzoate

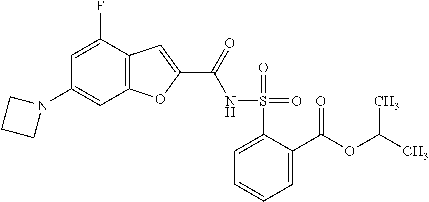 [SMILES: CC(C)OC(=O)c1ccccc1S(=O)(=O)NC(=O)c1cc2c(F)cc(N3CCC3)cc2o1]

Synthesized and purified analogously to

Example 99 using commercially available propan-2-yl 2-sulfamoylbenzoate (CAS: 14065-41-9, 96.1 mg, 395 μmol) instead of 5-acetyl-2-chlorobenzene-1-sulfonamide to give 22.0 mg (85% purity, 12% yield) of the title compound as a yellow solid. ¹H-NMR (400 MHz, DMSO-d₆) δ [ppm]: 1.137 (0.67), 1.210 (1.00), 1.227 (1.24), 1.264 (0.53), 1.280 (0.75), 1.292 (15.83), 1.307 (16.00), 2.084 (4.19), 2.291 (0.40), 2.309 (1.27), 2.327 (2.59), 2.346 (1.40), 2.364 (0.49), 2.518 (3.70), 2.522 (2.30), 2.539 (0.49), 2.665 (0.59), 2.669 (0.81), 2.673 (0.59), 3.878 (3.52), 3.896 (5.83), 3.915 (3.31), 5.121 (0.94), 5.136 (1.23), 5.152 (0.91), 6.315 (1.14), 6.319 (1.31), 6.354 (3.19), 7.637 (0.68), 7.654 (0.84), 7.727 (0.41), 7.746 (1.04), 7.758 (1.62), 8.119 (1.45), 8.125 (1.30), 8.135 (0.82), 8.139 (0.98), 8.142 (1.22); LC-MS (method 2): Rₜ=0.82 min; MS (ESIneg): m/z=459 [M−H]⁻

### Example 102

### Methyl 3-(2-{[6-(azetidin-1-yl)-4-fluoro-1-benzofuran-2-carbonyl]sulfamoyl}phenyl)propanoate

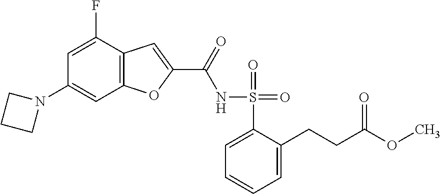 [SMILES: COC(=O)CCc1ccccc1S(=O)(=O)NC(=O)c1cc2c(F)cc(N3CCC3)cc2o1]

Synthesized and purified analogously to

Example 99 using methyl 3-(2-sulfamoylphenyl)propanoate (96.1 mg, 395 μmol) instead of 5-acetyl-2-chlorobenzene-1-sulfonamide to give 14.0 mg (8% yield) of the title compound as a yellow solid. ¹H-NMR (400 MHz, DMSO-d₆) δ [ppm]: −0.003 (0.68), 1.137 (3.41), 1.219 (0.49), 1.237 (2.27), 1.253 (2.16), 1.267 (1.35), 2.083 (15.32), 2.115 (1.58), 2.282 (0.55), 2.300 (1.71), 2.318 (2.89), 2.326 (2.05), 2.336 (2.30), 2.354 (0.64), 2.383 (0.41), 2.591 (2.12), 2.610 (2.83), 2.631 (2.24), 2.668 (1.58), 3.238 (0.52), 3.269 (2.60), 3.291 (3.75), 3.545 (16.00), 3.606 (1.95), 3.857 (3.83), 3.875 (6.83), 3.894 (3.78), 6.278 (1.02), 6.308 (1.03), 6.342 (3.92), 6.946 (1.38), 7.074 (1.44), 7.202 (1.39), 7.399 (1.38), 7.418 (1.04), 7.500 (0.98), 7.516 (0.69), 7.533 (0.61), 7.981 (0.92), 8.000 (0.92); LC-MS (method 1): Rₜ=1.27 min; MS (ESIneg): m/z=459 [M−H]⁻

### Example 103

### 6-(Azetidin-1-yl)-N-[2-chloro-5-(trifluoromethyl)pyridine-3-sulfonyl]-4-fluoro-1-benzofuran-2-carboxamide

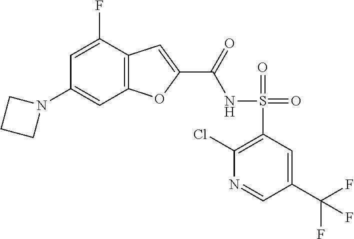 [SMILES: O=C(NS(=O)(=O)c1cc(C(F)(F)F)cnc1Cl)c1cc2c(F)cc(N3CCC3)cc2o1]

Synthesized and purified analogously to

Example 99 using commercially available 2-chloro-5-(trifluoromethyl)pyridine-3-sulfonamide (CAS: 1208081-89-3, 103 mg, 395 μmol) instead of 5-acetyl-2-chlorobenzene-1-sulfonamide to give 134 mg (85% yield) of the title compound as a yellow solid. ¹H-NMR (400 MHz, DMSO-d₆) δ [ppm]: 2.083 (16.00), 2.283 (0.40), 2.301 (1.29), 2.320 (2.08), 2.337 (1.53), 2.356 (0.45), 2.669 (0.59), 3.860 (3.60), 3.878 (6.10), 3.896 (3.45), 6.276 (1.37), 6.306 (1.41), 6.339 (2.85), 7.547 (1.35), 8.654 (1.94), 8.660 (2.06), 9.066 (1.82); LC-MS (method 2): Rₜ=0.86 min; MS (ESIpos): m/z=478 [M+H]⁺

### Example 104

### N-(2-amino-5-chloro-4,6-dimethylpyridine-3-sulfonyl)-6-(azetidin-1-yl)-4-fluoro-1-benzofuran-2-carboxamide

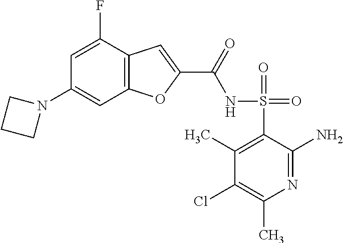 [SMILES: Cc1nc(N)c(S(=O)(=O)NC(=O)c2cc3c(F)cc(N4CCC4)cc3o2)c(C)c1Cl]

Synthesized and purified analogously to

Example 99 using 2-amino-5-chloro-4,6-dimethylpyridine-3-sulfonamide (37.0 mg, 157 μmol) instead of 5-acetyl-2-chlorobenzene-1-sulfonamide to give 22.0 mg (36% yield) of the title compound as an offwhite solid. ¹H-NMR (400 MHz, DMSO-d₆) δ [ppm]: 1.229 (0.57), 2.083 (6.70), 2.291 (0.48), 2.309 (1.50), 2.327 (2.70), 2.346 (1.71), 2.364 (0.63), 2.394 (16.00), 2.605 (15.47), 2.669 (0.64), 2.673 (0.52), 3.877 (4.07), 3.896 (7.02), 3.913 (4.04), 6.310 (1.76), 6.345 (4.11), 7.781 (0.97); LC-MS (method 1): Rₜ=1.21 min; MS (ESIpos): m/z=453 [M+H]⁺

### Example 105

### Methyl {[1-{2-[(2-ethoxybenzene-1-sulfonyl)carbamoyl]-4-fluoro-1-benzofuran-6-yl}azetidin-2-yl]methyl}carbamate (rac)

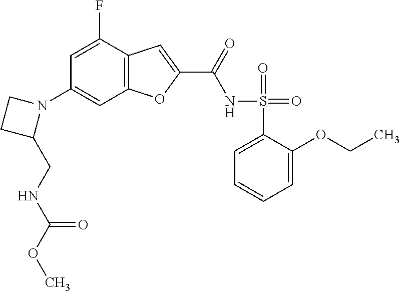 [SMILES: CCOc1ccccc1S(=O)(=O)NC(=O)c1cc2c(F)cc(N3CCC3CNC(=O)OC)cc2o1]

Synthesized and purified analogously to

Example 99 using 4-fluoro-6-[2-{[(methoxycarbonyl)amino]methyl}azetidin-1-yl]-1-benzofuran-2-carboxylic acid (rac) (Intermediate 31, 50.0 mg, 155 μmol) and 2-ethoxybenzene-1-sulfonamide (34.3 mg, 171 μmol) instead of 6-(azetidin-1-yl)-4-fluoro-1-benzofuran-2-carboxylic acid and 5-acetyl-2-chlorobenzene-1-sulfonamide to give 25.0 mg (29% yield) of the title compound as an offwhite solid. ¹H-NMR (400 MHz, DMSO-d₆) δ [ppm]: 1.152 (0.56), 1.170 (1.25), 1.187 (0.62), 1.212 (5.06), 1.223 (7.66), 1.229 (5.91), 1.240 (14.38), 1.258 (6.76), 2.084 (1.71), 2.146 (1.06), 2.327 (1.75), 2.331 (1.54), 2.523 (4.10), 2.665 (0.89), 2.669 (1.21), 2.673 (0.89), 2.889 (0.44), 3.255 (0.81), 3.273 (1.04), 3.290 (1.50), 3.387 (1.00), 3.398 (1.23), 3.413 (1.19), 3.423 (0.96), 3.434 (0.89), 3.533 (16.00), 3.547 (3.00), 3.585 (0.77), 3.604 (1.66), 3.625 (1.54), 3.645 (0.60), 3.934 (1.41), 3.944 (1.35), 4.106 (1.21), 4.134 (2.14), 4.151 (4.08), 4.168 (3.70), 4.185 (1.27), 5.759 (7.97), 6.394 (1.89), 6.423 (1.69), 6.485 (3.60), 6.520 (0.52), 7.108 (1.12), 7.127 (2.10), 7.146 (1.17), 7.201 (1.73), 7.221 (2.00), 7.412 (1.06), 7.427 (2.04), 7.441 (1.08), 7.519 (0.42), 7.619 (0.81), 7.638 (1.29), 7.657 (0.69), 7.889 (2.16), 7.892 (2.18), 7.908 (2.08), 7.912 (2.00), 8.049 (0.87), 12.458 (0.96); LC-MS (method 1): Rₜ=1.17 min; MS (ESIpos): m/z=506 [M+H]⁺

### Example 106

### 6-(Azetidin-1-yl)-4-fluoro-N-(4-hydroxynaphthalene-1-sulfonyl)-1-benzofuran-2-carboxamide

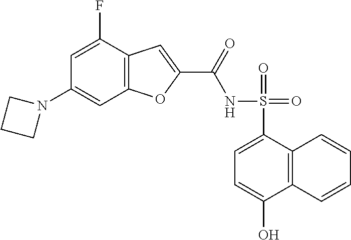 [SMILES: O=C(NS(=O)(=O)c1ccc(O)c2ccccc12)c1cc2c(F)cc(N3CCC3)cc2o1]

Synthesized and purified analogously to

Example 99 using 4-hydroxynaphthalene-1-sulfonamide (78.0 mg, 349 μmol) instead of 5-acetyl-2-chlorobenzene-1-sulfonamide to give 102 mg (77% yield) of the title compound as a yellow solid. ¹H-NMR (500 MHz, DMSO-d₆) δ [ppm]: 2.084 (16.00), 3.940 (0.66), 3.956 (1.08), 3.970 (0.65), 6.528 (0.49), 7.666 (0.56), 7.683 (0.59), 7.768 (0.95), 8.180 (0.75), 8.182 (0.71), 8.209 (0.62), 8.225 (0.52); LC-MS (method 1): Rₜ=1.36 min; MS (ESIneg): m/z=439 [M−H]⁻

### Example 107

### N-(2-Ethoxybenzene-1-sulfonyl)-6-(3-fluoroazetidin-1-yl)-1-benzofuran-2-carboxamide

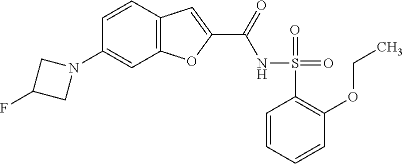 [SMILES: CCOc1ccccc1S(=O)(=O)NC(=O)c1cc2ccc(N3CC(F)C3)cc2o1]

Synthesized and purified analogously to Example 3 using 6-(3-fluoroazetidin-1-yl)-1-benzofuran-2-carboxylic acid

Intermediate 7, 65.0 mg, 276 μmol) and commercially available 2-ethoxybenzene-1-sulfonamide (CAS: 58734-61-5, 66.7 mg, 332 μmol) instead of 6-(azetidin-1-yl)-4-fluoro-1-benzofuran-2-carboxylic acid and 2-(cyclopentyloxy)-5-ethylbenzene-1-sulfonamide to give 46.0 mg (38% yield) of the title compound as a yellow solid. ¹H-NMR (400 MHz, DMSO-d₆) δ [ppm]: 0.000 (10.75), 1.222 (7.23), 1.239 (16.00), 1.257 (7.44), 2.077 (0.58), 2.521 (1.08), 2.526 (0.67), 3.925 (1.15), 3.929 (1.24), 3.949 (1.49), 3.954 (1.45), 3.956 (1.42), 3.982 (1.18), 3.985 (1.21), 3.990 (1.27), 4.009 (1.45), 4.014 (1.38), 4.131 (2.00), 4.149 (6.36), 4.166 (6.50), 4.183 (2.95), 4.194 (1.49), 4.196 (1.43), 4.204 (1.13), 4.206 (1.15), 4.218 (1.10), 4.221 (1.12), 4.232 (1.24), 4.234 (1.25), 4.246 (1.36), 4.249 (1.34), 4.256 (1.14), 4.259 (1.17), 4.270 (1.01), 4.273 (1.01), 5.425 (0.55), 5.432 (0.73), 5.440 (0.93), 5.447 (0.69), 5.454 (0.46), 5.569 (0.48), 5.576 (0.71), 5.584 (0.92), 5.591 (0.71), 5.598 (0.53), 6.547 (2.56), 6.552 (2.89), 6.568 (2.50), 6.573 (3.16), 6.613 (4.08), 7.113 (1.65), 7.116 (1.77), 7.134 (3.32), 7.152 (1.85), 7.154 (1.87), 7.203 (2.99), 7.223 (3.31), 7.603 (4.83), 7.620 (2.13), 7.625 (6.19), 7.639 (1.90), 7.643 (2.23), 7.646 (1.88), 7.660 (1.37), 7.664 (1.33), 7.896 (3.20), 7.900 (3.13), 7.916 (3.13), 7.920 (2.86), 7.991 (6.89), 7.993 (6.89), 12.401 (1.75); LC-MS (method 3): Rₜ=1.15 min; MS (ESIpos): m/z=419 [M+H]⁺

### Example 108

### 4-Fluoro-6-(3-fluoroazetidin-1-yl)-N-(5,6,7,8-tetrahydronaphthalene-1-sulfonyl)-1-benzofuran-2-carboxamide

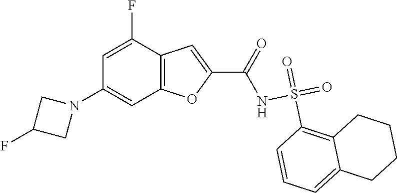 [SMILES: O=C(NS(=O)(=O)c1cccc2c1CCCC2)c1cc2c(F)cc(N3CC(F)C3)cc2o1]

Synthesized and purified analogously to Example 3 using 4-fluoro-6-(3-fluoroazetidin-1-yl)-1-benzofuran-2-carboxylic acid (Intermediate 18, 100 mg, 395 μmol) and 5,6,7,8-tetrahydronaphthalene-1-sulfonamide (100 mg, 474 μmol) instead of 6-(azetidin-1-yl)-4-fluoro-1-benzofuran-2-carboxylic acid and 2-(cyclopentyloxy)-5-ethylbenzene-1-sulfonamide to give 49.0 mg (26% yield) of the title compound as a yellow solid. ¹H-NMR (400 MHz, DMSO-d₆) δ [ppm]: 1.667 (1.09), 1.683 (2.83), 1.693 (4.65), 1.708 (6.22), 1.722 (6.42), 1.734 (6.11), 1.751 (4.70), 1.760 (2.82), 1.775 (1.03), 2.074 (8.83), 2.518 (1.44), 2.523 (0.92), 2.782 (4.90), 2.796 (9.34), 2.812 (4.71), 3.081 (4.98), 3.097 (9.38), 3.112 (4.71), 3.409 (0.75), 3.942 (2.94), 3.946 (3.17), 3.949 (2.90), 3.967 (3.84), 3.971 (3.80), 3.974 (3.70), 4.000 (2.94), 4.003 (3.13), 4.007 (3.25), 4.010 (2.91), 4.027 (3.77), 4.032 (3.58), 4.034 (3.27), 4.188 (3.04), 4.200 (3.67), 4.203 (3.58), 4.210 (2.86), 4.213 (2.94), 4.225 (2.80), 4.228 (2.83), 4.239 (3.17), 4.241 (3.21), 4.252 (3.53), 4.256 (3.45), 4.263 (2.86), 4.266 (2.97), 4.278 (2.60), 4.280 (2.58), 5.408 (0.72), 5.416 (1.43), 5.423 (1.95), 5.430 (2.44), 5.437 (1.82), 5.445 (1.22), 5.452 (0.57), 5.552 (0.64), 5.560 (1.24), 5.567 (1.89), 5.574 (2.39), 5.581 (1.93), 5.588 (1.40), 5.595 (0.65), 6.425 (6.11), 6.430 (6.94), 6.454 (5.78), 6.459 (7.22), 6.483 (10.67), 6.485 (12.39), 7.328 (3.75), 7.347 (9.92), 7.367 (7.26), 7.399 (7.64), 7.414 (3.98), 7.883 (6.41), 7.886 (6.57), 7.902 (6.07), 7.906 (5.80), 7.988 (16.00), 12.695 (0.44); LC-MS (method 3): Rₜ=1.37 min; MS (ESIpos): m/z=447 [M+H]⁺

### Example 109

### N-(2,3-dihydro-1,4-benzodioxine-5-sulfonyl)-4-fluoro-6-(3-fluoroazetidin-1-yl)-1-benzofuran-2-carboxamide

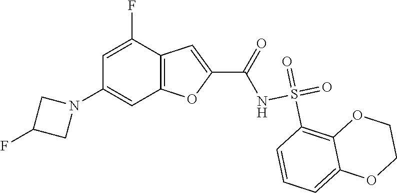 [SMILES: O=C(NS(=O)(=O)c1cccc2c1OCCO2)c1cc2c(F)cc(N3CC(F)C3)cc2o1]

Synthesized and purified analogously to Example 108 using 2,3-dihydro-1,4-benzodioxine-5-sulfonamide (102 mg, 474 μmol) instead of 5,6,7,8-tetrahydronaphthalene-1-sulfonamide to give 80.0 mg (38% yield) of the title compound 15 as a yellow solid. ¹H-NMR (400 MHz, DMSO-d₆) δ [ppm]: 1.740 (0.88), 2.075 (5.08), 2.518 (2.51), 2.523 (1.60), 2.540 (0.42), 3.082 (0.68), 3.093 (0.70), 3.942 (2.58), 3.946 (2.76), 3.950 (2.55), 3.967 (3.36), 3.971 (3.37), 3.974 (3.25), 4.000 (2.62), 4.003 (2.77), 4.007 (2.85), 4.010 (2.56), 4.027 (3.30), 4.032 (3.15), 4.190 (2.69), 4.202 (3.26), 4.205 (3.21), 4.212 (2.56), 4.215 (2.64), 4.226 (2.56), 4.230 (2.60), 4.240 (2.97), 4.243 (3.06), 4.269 (10.06), 4.278 (9.54), 4.282 (10.88), 4.313 (8.74), 4.318 (7.90), 4.326 (7.82), 4.333 (4.26), 5.411 (0.65), 5.419 (1.28), 5.427 (1.73), 5.433 (2.14), 5.441 (1.60), 5.448 (1.08), 5.456 (0.51), 5.555 (0.54), 5.563 (1.09), 5.570 (1.65), 5.577 (2.12), 5.584 (1.70), 5.591 (1.25), 5.599 (0.59), 6.430 (5.54), 6.433 (6.21), 6.459 (5.31), 6.462 (6.39), 6.496 (9.11), 6.499 (10.97), 6.502 (7.95), 7.000 (7.96), 7.020 (16.00), 7.040 (9.02), 7.184 (9.52), 7.188 (9.57), 7.204 (8.01), 7.209 (7.36), 7.446 (10.41), 7.450 (9.91), 7.466 (8.74), 7.470 (8.72), 8.046 (11.80), 12.635 (0.81); LC-MS (method 3): Rₜ=1.18 min; MS (ESIpos): m/z=451 [M+H]⁺

### Example 110

### 4-Fluoro-6-(3-fluoroazetidin-1-yl)-N-(1,2,3,4-tetrahydroquinoline-8-sulfonyl)-1-benzofuran-2-carboxamide

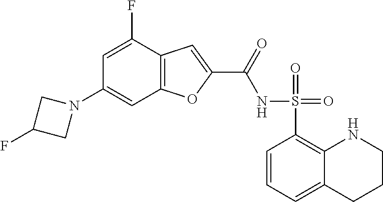 [SMILES: O=C(NS(=O)(=O)c1cccc2c1NCCC2)c1cc2c(F)cc(N3CC(F)C3)cc2o1]

Synthesized and purified analogously to Example 108 using 1,2,3,4-tetrahydroquinoline-8-sulfonamide (101 mg, 474 μmol) instead of 5,6,7,8-tetrahydronaphthalene-1-sulfonamide to give 57.0 mg (31% yield) of the title compound as a yellow solid. ¹H-NMR (400 MHz, DMSO-d₆) δ [ppm]: 1.745 (1.61), 1.761 (4.69), 1.775 (6.30), 1.788 (4.99), 1.803 (1.84), 2.074 (1.00), 2.518 (5.66), 2.523 (3.94), 2.708 (5.34), 2.723 (9.16), 2.739 (5.12), 3.336 (7.69), 3.350 (10.21), 3.363 (8.18), 3.951 (3.04), 3.955 (3.22), 3.976 (4.00), 3.980 (3.92), 3.983 (3.74), 4.009 (3.04), 4.012 (3.24), 4.016 (3.31), 4.037 (3.90), 4.041 (3.72), 4.196 (3.04), 4.208 (3.76), 4.212 (3.69), 4.222 (2.96), 4.233 (2.87), 4.236 (2.89), 4.247 (3.33), 4.261 (3.58), 4.264 (3.50), 4.272 (3.00), 4.275 (3.04), 4.286 (2.65), 5.415 (0.72), 5.423 (1.42), 5.430 (1.99), 5.437 (2.41), 5.444 (1.88), 5.451 (1.27), 5.559 (0.64), 5.566 (1.29), 5.573 (1.89), 5.581 (2.42), 5.588 (1.95), 5.595 (1.43), 5.602 (0.68), 6.434 (6.23), 6.438 (6.73), 6.463 (5.82), 6.467 (7.45), 6.485 (10.92), 6.487 (12.60), 6.523 (6.89), 6.542 (8.99), 6.544 (8.89), 6.562 (7.38), 7.114 (5.89), 7.117 (5.97), 7.132 (5.72), 7.136 (5.49), 7.515 (6.72), 7.519 (6.80), 7.535 (6.74), 7.539 (6.17), 7.844 (15.96), 7.846 (16.00); LC-MS (method 3): Rₜ=1.32 min; MS (ESIpos): m/z=448 [M+H]⁺

### Example 111

### 4-Fluoro-6-(3-fluoroazetidin-1-yl)-N-(quinoline-4-sulfonyl)-1-benzofuran-2-carboxamide

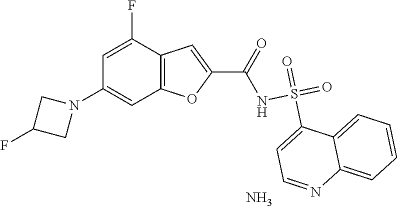

Synthesized analogously to Example 108 using quinoline-4-sulfonamide (98.7 mg, 474 μmol) instead of 5,6,7,8-tetrahydronaphthalene-1-sulfonamide and purified by HT-HPLC (basic method) to give 12.0 mg (6% yield, could contain ammonium salt) of the title compound as an ochre solid. ¹H-NMR (400 MHz, DMSO-d₆) δ [ppm]: 2.318 (0.99), 2.337 (1.04), 2.518 (11.01), 2.523 (7.36), 2.540 (11.88), 2.679 (1.04), 3.394 (7.59), 3.888 (1.80), 3.893 (1.86), 3.913 (2.26), 3.917 (2.20), 3.920 (2.14), 3.946 (1.86), 3.953 (1.97), 3.957 (1.74), 3.973 (2.26), 4.142 (1.74), 4.154 (2.32), 4.157 (2.14), 4.163 (1.68), 4.178 (1.68), 4.195 (1.80), 4.206 (2.14), 4.219 (1.80), 4.234 (1.45), 5.396 (0.81), 5.404 (1.10), 5.411 (1.45), 5.418 (1.04), 5.540 (0.87), 5.547 (0.99), 5.555 (1.45), 5.562 (1.04), 5.569 (0.81), 6.323 (3.25), 6.327 (3.48), 6.352 (3.25), 6.356 (3.36), 6.448 (5.86), 6.450 (7.01), 6.455 (4.87), 6.945 (15.07), 7.072 (16.00), 7.200 (15.42), 7.235 (1.16), 7.641 (1.51), 7.662 (2.84), 7.680 (1.97), 7.756 (1.80), 7.760 (2.03), 7.778 (3.30), 7.782 (2.32), 7.795 (1.80), 8.012 (4.17), 8.022 (4.17), 8.076 (3.77), 8.084 (0.93), 8.086 (0.93), 8.097 (3.36), 8.765 (3.48), 8.768 (3.59), 8.787 (3.54), 9.049 (5.51), 9.060 (5.28); LC-MS (method 1): Rₜ=0.98 min; MS (ESIpos): m/z=444 [M+H]⁺

### Example 112

### 4-Fluoro-6-(3-fluoroazetidin-1-yl)-N-(2-methyl-1,2,3,4-tetrahydroisoquinoline-5-sulfonyl)-1-benzofuran-2-carboxamide

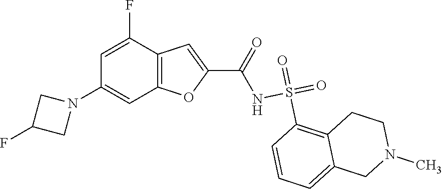 [SMILES: CN1CCc2c(cccc2S(=O)(=O)NC(=O)c2cc3c(F)cc(N4CC(F)C4)cc3o2)C1]

Synthesized and purified analogously to Example 111 using 2-methyl-1,2,3,4-tetrahydroisoquinoline-5-sulfonamide (107 mg, 474 μmol) instead of quinoline-4-sulfonamide to give 17.0 mg (9% yield) of the title compound as an ochre solid. ¹H-NMR (400 MHz, DMSO-d₆) δ [ppm]: 2.075 (1.25), 2.327 (1.93), 2.540 (0.81), 2.669 (1.74), 2.863 (16.00), 3.677 (1.15), 3.887 (2.30), 3.905 (2.74), 3.948 (2.33), 3.966 (2.68), 4.139 (2.21), 4.153 (2.61), 4.162 (2.12), 4.178 (2.12), 4.191 (2.33), 4.204 (2.61), 4.215 (2.21), 4.229 (1.96), 4.379 (1.15), 5.422 (1.59), 5.565 (1.59), 6.308 (3.86), 6.311 (3.98), 6.336 (3.89), 6.341 (4.02), 6.463 (8.06), 7.036 (6.04), 7.232 (3.30), 7.252 (4.45), 7.325 (3.46), 7.344 (5.73), 7.364 (2.46), 7.873 (4.42), 7.891 (4.02), 9.761 (1.68); LC-MS (method 1): Rₜ=0.78 min; MS (ESIpos): m/z=462 [M+H]⁺

### Example 113

### N-(5-tert-butyl-2-ethoxybenzene-1-sulfonyl)-4-fluoro-6-(3-fluoroazetidin-1-yl)-1-benzofuran-2-carboxamide

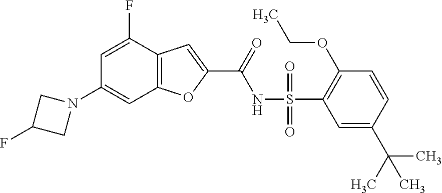 [SMILES: CCOc1ccc(C(C)(C)C)cc1S(=O)(=O)NC(=O)c1cc2c(F)cc(N3CC(F)C3)cc2o1]

Synthesized and purified analogously to Example 108 using 5-tert-butyl-2-ethoxybenzene-1-sulfonamide (244 mg, 948 μmol) instead of 5,6,7,8-tetrahydronaphthalene-1-sulfonamide to give 115 mg (28% yield) of the title compound as an offwhite solid. ¹H-NMR (400 MHz, DMSO-d₆) δ [ppm]: 1.211 (1.18), 1.228 (2.70), 1.246 (1.28), 1.299 (16.00), 4.123 (1.09), 4.141 (1.08), 6.429 (0.40), 6.433 (0.48), 6.462 (0.58), 6.471 (0.94), 7.133 (0.72), 7.155 (0.79), 7.659 (0.48), 7.666 (0.51), 7.681 (0.43), 7.687 (0.45), 7.850 (1.05), 7.856 (0.99), 8.068 (0.95); LC-MS (method 1): Rₜ=1.40 min; MS (ESIpos): m/z=493 [M+H]⁺

### Example 114

### N-[2-(dichloromethyl)benzene-1-sulfonyl]-4-fluoro-6-(3-fluoroazetidin-1-yl)-1-benzofuran-2-carboxamide

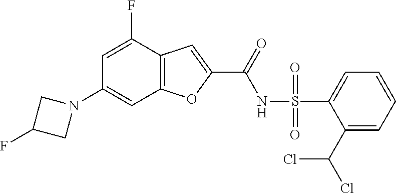 [SMILES: O=C(NS(=O)(=O)c1ccccc1C(Cl)Cl)c1cc2c(F)cc(N3CC(F)C3)cc2o1]

Synthesized and purified analogously to Example 108 using 2-(dichloromethyl)benzene-1-sulfonamide (114 mg, 474 μmol) instead of 5,6,7,8-tetrahydronaphthalene-1-sulfonamide to give 71.0 mg (36% yield) of the title compound as a yellow solid. ¹H-NMR (400 MHz, DMSO-d₆) δ [ppm]: 2.074 (6.67), 2.518 (2.09), 2.523 (1.39), 3.941 (1.69), 3.946 (1.82), 3.949 (1.68), 3.966 (2.21), 3.970 (2.21), 3.974 (2.14), 3.999 (1.72), 4.002 (1.81), 4.007 (1.86), 4.010 (1.68), 4.027 (2.18), 4.031 (2.07), 4.188 (1.76), 4.199 (2.13), 4.202 (2.08), 4.210 (1.66), 4.213 (1.71), 4.225 (1.63), 4.228 (1.64), 4.238 (1.86), 4.241 (1.87), 4.252 (2.05), 4.256 (2.00), 4.263 (1.69), 4.266 (1.76), 4.277 (1.50), 4.280 (1.49), 5.408 (0.44), 5.416 (0.84), 5.423 (1.15), 5.430 (1.44), 5.438 (1.08), 5.445 (0.73), 5.560 (0.76), 5.567 (1.11), 5.574 (1.42), 5.581 (1.14), 5.588 (0.83), 5.595 (0.40), 6.423 (3.59), 6.427 (3.93), 6.452 (3.32), 6.456 (4.22), 6.478 (6.22), 6.481 (7.26), 6.485 (5.06), 7.691 (2.11), 7.694 (2.18), 7.711 (3.76), 7.713 (3.90), 7.730 (2.87), 7.733 (2.77), 7.820 (5.71), 7.863 (1.98), 7.867 (2.11), 7.882 (3.53), 7.886 (3.70), 7.901 (1.95), 7.905 (1.93), 7.979 (16.00), 8.038 (4.67), 8.041 (4.73), 8.058 (4.23), 8.061 (4.06), 8.111 (4.50), 8.114 (4.77), 8.131 (3.95), 8.133 (3.75); LC-MS (method 3): Rₜ=1.58 min; MS (ESIpos): m/z=475 [M+H]⁺

### Example 115

### 4-Fluoro-6-(3-fluoroazetidin-1-yl)-N-[4-(2-methyl-1,3-thiazol-4-yl)benzene-1-sulfonyl]-1-benzofuran-2-carboxamide

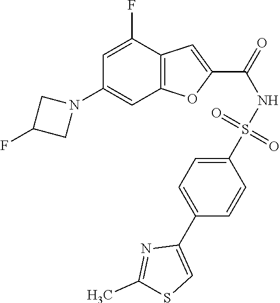 [SMILES: Cc1nc(-c2ccc(S(=O)(=O)NC(=O)c3cc4c(F)cc(N5CC(F)C5)cc4o3)cc2)cs1]

Synthesized and purified analogously to Example 108 using 4-(2-methyl-1,3-thiazol-4-yl)benzene-1-sulfonamide (121 mg, 474 μmol) instead of 5,6,7,8-tetrahydronaphthalene-1-sulfonamide to give 10.0 mg (5% yield) of the title compound as a yellow solid. ¹H-NMR (400 MHz, DMSO-d₆) δ [ppm]: 1.211 (0.91), 1.228 (0.90), 2.518 (0.76), 2.523 (0.49), 2.732 (16.00), 3.369 (0.67), 3.941 (0.56), 3.945 (0.61), 3.965 (0.67), 3.970 (0.68), 3.973 (0.68), 4.002 (0.58), 4.006 (0.62), 4.026 (0.65), 4.031 (0.64), 4.185 (0.55), 4.198 (0.64), 4.201 (0.63), 4.209 (0.53), 4.223 (0.53), 4.226 (0.50), 4.237 (0.59), 4.251 (0.63), 4.254 (0.60), 4.261 (0.54), 4.264 (0.54), 4.276 (0.45), 5.428 (0.44), 5.571 (0.43), 6.419 (1.09), 6.423 (1.22), 6.448 (1.03), 6.452 (1.26), 6.478 (1.85), 6.480 (2.21), 6.484 (1.53), 7.902 (2.34), 8.022 (0.42), 8.028 (3.34), 8.032 (1.06), 8.045 (1.24), 8.049 (4.20), 8.054 (0.69), 8.165 (0.68), 8.169 (4.22), 8.174 (1.23), 8.186 (1.15), 8.191 (3.61), 8.194 (7.42); LC-MS (method 3): Rₜ=1.32 min; MS (ESIneg): m/z=488 [M−H]⁻

### Example 116

### N-(2-Ethoxybenzene-1-sulfonyl)-6-(3-fluoro-3-methylazetidin-1-yl)-1-benzofuran-2-carboxamide

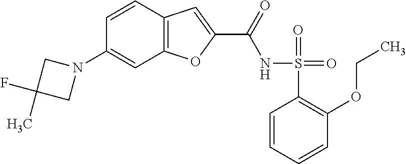 [SMILES: CCOc1ccccc1S(=O)(=O)NC(=O)c1cc2ccc(N3CC(C)(F)C3)cc2o1]

Synthesized and purified analogously to Example 3 using 6-(3-fluoro-3-methylazetidin-1-yl)-1-benzofuran-2-carboxylic acid (Intermediate 9, 25.0 mg, 100 μmol) and commercially available 2-ethoxybenzene-1-sulfonamide (CAS: 58734-61-5, 24.2 mg, 120 μmol) instead of 6-(azetidin-1-yl)-4-fluoro-1-benzofuran-2-carboxylic acid and 2-(cyclopentyloxy)-5-ethylbenzene-1-sulfonamide to give 9.00 mg (19% yield) of the title compound as a light yellow solid. ¹H-NMR (400 MHz, DMSO-d₆) δ [ppm]: 1.140 (0.50), 1.220 (7.05), 1.238 (16.00), 1.255 (7.30), 1.605 (8.05), 1.660 (7.96), 2.086 (1.25), 2.521 (2.54), 2.525 (1.59), 3.947 (0.66), 3.969 (4.96), 3.978 (4.41), 3.992 (1.14), 4.016 (4.17), 4.029 (3.99), 4.052 (0.85), 4.131 (1.88), 4.148 (6.00), 4.166 (6.06), 4.183 (1.78), 6.550 (2.43), 6.555 (2.70), 6.571 (2.31), 6.576 (2.81), 6.615 (3.77), 7.115 (1.60), 7.133 (3.07), 7.151 (1.70), 7.153 (1.70), 7.203 (2.77), 7.224 (3.00), 7.602 (4.43), 7.624 (5.38), 7.642 (2.01), 7.659 (1.15), 7.663 (1.13), 7.893 (3.00), 7.898 (2.96), 7.913 (2.95), 7.917 (2.69), 7.987 (3.76), 12.399 (1.48); LC-MS (method 7): Rₜ=1.18 min; MS (ESIpos): m/z=433 [M+H]⁺

### Example 117

### N-(2-Ethoxybenzene-1-sulfonyl)-6-[3-(trifluoromethyl)azetidin-1-yl]-1-benzofuran-2-carboxamide

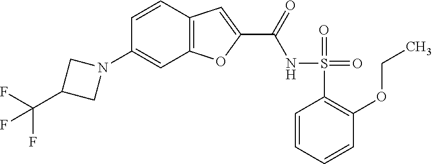 [SMILES: CCOc1ccccc1S(=O)(=O)NC(=O)c1cc2ccc(N3CC(C(F)(F)F)C3)cc2o1]

Synthesized and purified analogously to Example 3 using 6-[3-(trifluoromethyl)azetidin-1-yl]-1-benzofuran-2-carboxylic acid (Intermediate 11, 95.0 mg, 333 μmol) and commercially available 2-ethoxybenzene-1-sulfonamide (CAS: 58734-61-5, 80.4 mg, 400 μmol) instead of 6-(azetidin-1-yl)-4-fluoro-1-benzofuran-2-carboxylic acid and 2-(cyclopentyloxy)-5-ethylbenzene-1-sulfonamide to give 2.00 mg (% yield) of the title compound as a white solid. ¹H-NMR (400 MHz, DMSO-d₆) δ [ppm]: 1.218 (7.37), 1.235 (16.00), 1.253 (7.53), 2.075 (1.95), 2.518 (7.89), 2.523 (5.52), 2.540 (1.78), 3.736 (0.72), 3.924 (2.85), 3.936 (2.71), 3.945 (3.62), 3.958 (2.92), 4.101 (3.44), 4.123 (6.62), 4.145 (6.80), 4.162 (4.76), 4.180 (1.46), 6.558 (2.23), 6.563 (2.36), 6.579 (2.16), 6.583 (2.50), 6.644 (3.89), 7.109 (1.27), 7.129 (2.46), 7.148 (1.36), 7.199 (2.12), 7.221 (2.26), 7.613 (3.95), 7.634 (4.47), 7.656 (0.80), 7.889 (2.52), 7.894 (2.54), 7.910 (2.44), 7.913 (2.24), 7.987 (1.69), 12.410 (1.64); LC-MS (method 1): Rₜ=1.26 min; MS (ESIpos): m/z=469 [M+H]⁺

### Example 118

### 6-(3,3-Difluoroazetidin-1-yl)-N-(2-ethoxybenzene-1-sulfonyl)-1-benzofuran-2-carboxamide

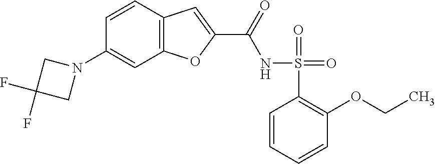 [SMILES: CCOc1ccccc1S(=O)(=O)NC(=O)c1cc2ccc(N3CC(F)(F)C3)cc2o1]

Synthesized and purified analogously to Example 3 using 6-(3,3-difluoroazetidin-1-yl)-1-benzofuran-2-carboxylic acid (Intermediate 13, 85.0 mg, 336 μmol) and commercially available 2-ethoxybenzene-1-sulfonamide (CAS: 58734-61-5, 81.1 mg, 403 μmol) instead of 6-(azetidin-1-yl)-4-fluoro-1-benzofuran-2-carboxylic acid and 2-(cyclopentyloxy)-5-ethylbenzene-1-sulfonamide to give 10.0 mg (6% yield) of the title compound as a white solid. ¹H-NMR (400 MHz, DMSO-d₆) δ [ppm]: 1.220 (6.46), 1.237 (16.00), 1.254 (6.74), 2.077 (2.70), 2.521 (1.63), 2.525 (0.98), 2.672 (0.45), 4.133 (1.76), 4.151 (5.78), 4.168 (5.76), 4.185 (1.67), 4.328 (4.73), 4.359 (10.42), 4.390 (4.31), 6.632 (2.61), 6.637 (2.68), 6.653 (2.57), 6.659 (2.86), 6.747 (3.29), 6.752 (2.94), 7.116 (1.54), 7.118 (1.54), 7.136 (2.72), 7.154 (1.70), 7.156 (1.68), 7.206 (2.47), 7.226 (2.77), 7.624 (1.55), 7.628 (1.63), 7.642 (1.73), 7.646 (1.98), 7.649 (1.74), 7.658 (4.34), 7.663 (1.48), 7.668 (1.33), 7.680 (3.96), 7.898 (3.05), 7.902 (3.00), 7.917 (2.93), 7.922 (2.66), 8.014 (5.09), 8.016 (5.01), 12.463 (1.01); LC-MS (method 3): Rₜ=1.17 min; MS (ESIpos): m/z=437 [M+H]⁺

### Example 119

### N-([1,1′-biphenyl]-2-sulfonyl)-6-(3,3-difluoroazetidin-1-yl)-1-benzofuran-2-carboxamide

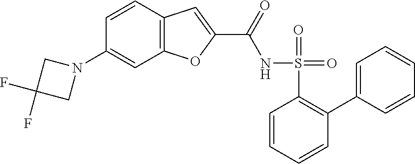 [SMILES: O=C(NS(=O)(=O)c1ccccc1-c1ccccc1)c1cc2ccc(N3CC(F)(F)C3)cc2o1]

Synthesized and purified analogously to Example 3 using 6-(3,3-difluoroazetidin-1-yl)-1-benzofuran-2-carboxylic acid (Intermediate 13, 85.0 mg, 336 μmol) and commercially available [1,1′-biphenyl]-2-sulfonamide (CAS: 40182-06-7, 94.0 mg, 403 μmol) instead of 6-(azetidin-1-yl)-4-fluoro-1-benzofuran-2-carboxylic acid and 2-(cyclopentyloxy)-5-ethylbenzene-1-sulfonamide to give the title compound as a yellow solid. ¹H-NMR (400 MHz, DMSO-d₆) δ [ppm]: 2.077 (0.96), 2.521 (1.12), 2.526 (0.68), 3.390 (0.77), 4.339 (7.24), 4.369 (16.00), 4.400 (6.57), 6.630 (3.91), 6.635 (4.15), 6.652 (3.77), 6.657 (4.25), 6.738 (5.27), 6.742 (4.76), 7.242 (3.97), 7.246 (6.03), 7.251 (1.77), 7.259 (4.08), 7.263 (9.32), 7.266 (7.58), 7.271 (1.28), 7.292 (3.37), 7.296 (5.26), 7.301 (1.62), 7.313 (12.65), 7.317 (4.16), 7.328 (6.43), 7.332 (8.09), 7.353 (2.34), 7.356 (4.19), 7.360 (2.21), 7.368 (1.44), 7.375 (4.07), 7.381 (0.89), 7.389 (0.80), 7.392 (1.22), 7.396 (0.63), 7.627 (8.31), 7.629 (8.14), 7.631 (7.88), 7.648 (7.45), 7.664 (3.72), 7.667 (3.49), 7.684 (3.13), 7.688 (2.76), 7.714 (3.11), 7.717 (3.38), 7.733 (4.39), 7.736 (4.11), 7.751 (1.73), 7.755 (1.62), 8.162 (4.31), 8.165 (4.62), 8.182 (3.95), 8.185 (3.70); LC-MS (method 3): Rₜ=1.29 min; MS (ESIpos): m/z=469 [M+H]⁺

### Example 120

### N-(2-ethoxybenzene-1-sulfonyl)-6-(3-phenylazetidin-1-yl)-1-benzofuran-2-carboxamide

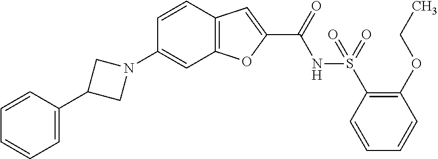 [SMILES: CCOc1ccccc1S(=O)(=O)NC(=O)c1cc2ccc(N3CC(c4ccccc4)C3)cc2o1]

Synthesized and purified analogously to Example 3 using 6-(3-phenylazetidin-1-yl)-1-benzofuran-2-carboxylic acid (Intermediate 33, 15.0 mg, 51.1 μmol) and 2-ethoxybenzene-1-sulfonamide (12.3 mg, 61.4 μmol) instead of 6-(azetidin-1-yl)-4-fluoro-1-benzofuran-2-carboxylic acid and 2-(cyclopentyloxy)-5-ethylbenzene-1-sulfonamide to give 8.00 mg (30% yield) of the title compound as an ochre solid. ¹H-NMR (400 MHz, DMSO-d₆) δ [ppm]: 1.221 (6.31), 1.238 (12.62), 1.256 (6.22), 2.326 (5.07), 2.522 (16.00), 2.668 (4.98), 3.866 (3.02), 3.882 (5.42), 3.899 (4.18), 3.972 (1.60), 3.987 (2.22), 4.159 (2.58), 4.310 (3.73), 4.330 (6.93), 4.349 (3.29), 6.568 (1.87), 6.603 (6.76), 7.118 (1.42), 7.235 (1.87), 7.253 (4.00), 7.271 (2.84), 7.336 (3.47), 7.356 (8.44), 7.374 (8.36), 7.386 (10.67), 7.403 (4.00), 7.614 (2.49), 7.886 (1.78), 7.991 (1.24), 12.374 (2.49); LC-MS (method 3): Rₜ=1.42 min; MS (ESIpos): m/z=477 [M+H]⁺

### Example 121

### 6-(3-Ethoxyazetidin-1-yl)-N-(2-ethoxybenzene-1-sulfonyl)-1-benzofuran-2-carboxamide

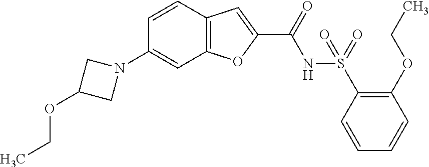 [SMILES: CCOc1ccccc1S(=O)(=O)NC(=O)c1cc2ccc(N3CC(OCC)C3)cc2o1]

Synthesized and purified analogously to Example 120 using 6-(3-ethoxyazetidin-1-yl)-1-benzofuran-2-carboxylic acid (Intermediate 35, 70.0 mg, 268 μmol) instead of 6-(3-phenylazetidin-1-yl)-1-benzofuran-2-carboxylic acid to give 3.80 mg (85% purity, 3% yield) of the title compound as a light brown solid. ¹H-NMR (400 MHz, CHLOROFORM-d) δ [ppm]: 0.849 (1.03), 0.887 (0.50), 1.249 (7.58), 1.266 (16.00), 1.284 (7.85), 1.342 (2.90), 1.423 (6.61), 1.431 (2.57), 1.441 (13.16), 1.458 (6.52), 2.139 (1.15), 2.182 (0.41), 2.795 (0.58), 3.509 (2.39), 3.526 (6.93), 3.544 (6.87), 3.561 (2.25), 3.811 (3.43), 3.822 (3.83), 3.831 (4.07), 3.843 (3.80), 4.124 (2.10), 4.141 (6.25), 4.159 (6.18), 4.176 (2.19), 4.190 (3.57), 4.207 (5.21), 4.225 (3.49), 4.455 (0.67), 4.467 (1.51), 4.470 (1.52), 4.481 (2.32), 4.493 (1.34), 4.508 (0.50), 6.446 (4.94), 6.470 (3.00), 6.475 (2.35), 6.492 (2.96), 6.496 (2.55), 6.955 (3.36), 6.975 (3.59), 7.091 (1.77), 7.110 (3.58), 7.129 (1.99), 7.437 (5.18), 7.444 (7.79), 7.457 (4.41), 7.535 (1.54), 7.539 (1.58), 7.556 (2.61), 7.574 (1.35), 7.578 (1.29), 8.141 (2.83), 8.144 (2.85), 8.160 (2.78), 8.164 (2.67); LC-MS (method 1): Rₜ=1.22 min; MS (ESIneg): m/z=443 [M−H]⁻

### Example 122

### 6-(5-Azaspiro[2.3]hexan-5-yl)-N-(2-ethoxybenzene-1-sulfonyl)-1-benzofuran-2-carboxamide

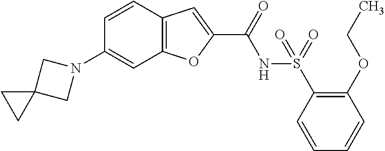 [SMILES: CCOc1ccccc1S(=O)(=O)NC(=O)c1cc2ccc(N3CC4(CC4)C3)cc2o1]

Synthesized and purified analogously to Example 120 using 6-(5-azaspiro[2.3]hexan-5-yl)-1-benzofuran-2-carboxylic acid (Intermediate 37, 35.0 mg, 144 μmol) instead of 6-(3-phenylazetidin-1-yl)-1-benzofuran-2-carboxylic acid to give 5.00 mg (95% purity, 8% yield) of the title compound as a green solid. ¹H-NMR (400 MHz, DMSO-d₆) δ [ppm]: 0.669 (14.51), 1.223 (3.92), 1.240 (8.74), 1.258 (3.96), 2.522 (0.93), 3.836 (0.69), 3.970 (16.00), 4.128 (1.04), 4.145 (3.17), 4.162 (3.16), 4.180 (0.97), 6.499 (1.24), 6.504 (1.70), 6.524 (5.74), 7.107 (0.86), 7.126 (1.70), 7.145 (0.92), 7.197 (1.51), 7.218 (1.64), 7.570 (1.91), 7.592 (1.71), 7.612 (0.68), 7.617 (0.71), 7.634 (1.14), 7.652 (0.56), 7.656 (0.54), 7.887 (1.62), 7.892 (1.65), 7.908 (1.54), 7.912 (1.47), 7.968 (1.15), 12.348 (1.21); LC-MS (method 3): Rₜ=1.34 min; MS (ESIpos): m/z=427 [M+H]⁺

### Example 123

### 6-(2-Azaspiro[3.3]heptan-2-yl)-N-(2-ethoxybenzene-1-sulfonyl)-1-benzofuran-2-carboxamide

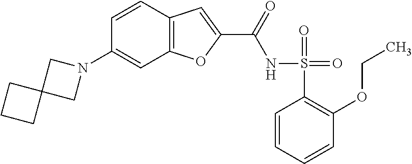 [SMILES: CCOc1ccccc1S(=O)(=O)NC(=O)c1cc2ccc(N3CC4(CCC4)C3)cc2o1]

Synthesized and purified analogously to Example 120 using 6-(2-azaspiro[3.3]heptan-2-yl)-1-benzofuran-2-carboxylic acid (Intermediate 39, 75.0 mg, 291 μmol) instead of 6-(3-phenylazetidin-1-yl)-1-benzofuran-2-carboxylic acid to give 7.00 mg (5% yield) of the title compound as a yellow solid. ¹H-NMR (400 MHz, DMSO-d₆) δ [ppm]: 1.167 (0.60), 1.216 (4.43), 1.233 (9.52), 1.251 (4.58), 1.797 (1.06), 1.817 (1.95), 1.834 (1.38), 1.856 (0.48), 2.155 (3.78), 2.174 (5.75), 2.192 (3.03), 2.332 (0.99), 2.518 (4.91), 2.523 (3.42), 3.848 (16.00), 4.123 (0.90), 4.140 (2.85), 4.158 (2.81), 4.175 (0.88), 6.462 (1.09), 6.466 (1.80), 6.485 (5.20), 7.107 (0.76), 7.125 (1.44), 7.143 (0.83), 7.194 (1.24), 7.215 (1.36), 7.544 (1.96), 7.567 (1.94), 7.616 (0.55), 7.634 (0.90), 7.652 (0.49), 7.885 (1.53), 7.889 (1.54), 7.905 (1.48), 7.908 (1.36), 7.955 (1.08), 12.338 (1.18); LC-MS (method 1): Rₜ=1.39 min; MS (ESIpos): m/z=441 [M+H]⁺

### Example 124

### 6-(3,3-dimethylazetidin-1-yl)-N-(2-ethoxybenzene-1-sulfonyl)-1-benzofuran-2-carboxamide

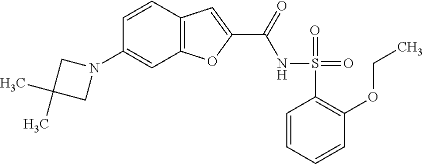 [SMILES: CCOc1ccccc1S(=O)(=O)NC(=O)c1cc2ccc(N3CC(C)(C)C3)cc2o1]

Synthesized and purified analogously to Example 120 using 6-(3,3-dimethylazetidin-1-yl)-1-benzofuran-2-carboxylic acid (Intermediate 41, 35.0 mg, 143 μmol) instead of 6-(3-phenylazetidin-1-yl)-1-benzofuran-2-carboxylic acid to give 12.0 mg (95% purity, 19% yield) of the title compound as a yellow solid. ¹H-NMR (400 MHz, DMSO-d6) δ[ppm]: 1.217 (2.75), 1.234 (6.44), 1.252 (2.94), 1.284 (16.00), 2.518 (2.19), 2.523 (1.45), 3.578 (0.91), 3.592 (10.83), 4.124 (0.55), 4.142 (1.69), 4.159 (1.67), 4.177 (0.53), 6.467 (0.67), 6.471 (1.21), 6.480 (1.51), 6.483 (1.64), 6.489 (1.38), 6.494 (0.54), 7.105 (0.44), 7.124 (0.87), 7.143 (0.48), 7.195 (0.73), 7.216 (0.80), 7.544 (1.31), 7.567 (1.21), 7.633 (0.54), 7.884 (0.91), 7.889 (0.92), 7.905 (0.89), 7.908 (0.82), 7.960 (0.57), 12.331 (0.77); LC-MS (method 6): Rₜ=1.32 min; MS (ESIpos): m/z=429 [M+H]⁺

### Example 125

### N-(2-ethoxybenzene-1-sulfonyl)-6-(2-oxa-6-azaspiro[3.3]heptan-6-yl)-1-benzofuran-2-carboxamide

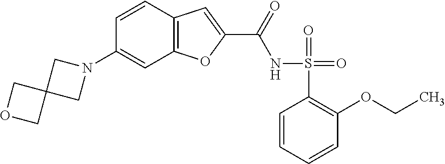 [SMILES: CCOc1ccccc1S(=O)(=O)NC(=O)c1cc2ccc(N3CC4(COC4)C3)cc2o1]

Synthesized and purified analogously to Example 120 using 6-(2-oxa-6-azaspiro[3.3]heptan-6-yl)-1-benzofuran-2-carboxylic acid (Intermediate 42, 11.0 mg, 42.4 μmol) instead of 6-(3-phenylazetidin-1-yl)-1-benzofuran-2-carboxylic acid to give 4.00 mg (20% yield) of the title compound as an offwhite solid. ¹H-NMR (400 MHz, DMSO-d₆) δ [ppm]: 1.215 (4.05), 1.232 (9.27), 1.250 (4.19), 2.077 (4.34), 2.521 (6.64), 2.525 (4.32), 4.057 (16.00), 4.124 (0.90), 4.142 (2.72), 4.159 (2.74), 4.177 (0.85), 4.720 (15.58), 4.729 (1.51), 6.495 (1.25), 6.499 (1.49), 6.516 (1.14), 6.521 (1.69), 6.538 (2.65), 7.109 (0.75), 7.127 (1.47), 7.146 (0.81), 7.196 (1.25), 7.217 (1.36), 7.567 (2.04), 7.589 (1.95), 7.618 (0.57), 7.636 (0.94), 7.887 (1.45), 7.892 (1.49), 7.907 (1.42), 7.911 (1.36), 7.964 (0.96), 12.364 (1.12), LC-MS (method 1): Rₜ=1.05 min; MS (ESIpos): m/z=443 [M+H]⁺

### Example 126

### Sodium [6-(azetidin-1-yl)-4-fluoro-1-benzofuran-2-carbonyl](2-ethoxybenzene-1-sulfonyl)azanide

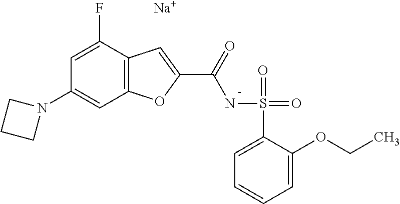 [SMILES: [Na+] ; CCOc1ccccc1S(=O)(=O)NC(=O)c1cc2c(F)cc(N3CCC3)cc2o1]

### 6-(Azetidin-1-yl)-4-fluoro-1-benzofuran-2-carboxylic acid

Intermediate 3, 1.00 g, 4.25 mmol) and CDI (813 mg, 5.02 mmol; CAS-RN:[530-62-1]) were dissolved in THF and stirred at room temperature. After 1 h, 2-ethoxybenzene-1-sulfonamide (1.11 g, 5.53 mmol) was added followed by DBU (880 μL, 5.9 mmol; CAS-RN:[6674-22-2]) and the resulting solution was stirred at room temperature for 15 min. The reaction mixture was concentrated in vacuo, the residue was suspended in 20 mL aq. HCl (1 M) and stirred for 10 min. The undissolved solid was collected by filtration, dissolved in dichloromethane/iso-propanol 8/2 and washed with 20 mL aq. NaOH (2 M). The organic layer was dried over sodium sulfate and concentrated in vacuo to give an brownish solid. 100 mL of hot ethanol/water 9/1 was added to the crude product and heated under reflux until the solid was completely dissolved. After cooling to room temperature 50% of the volume was evaporated and the remaining suspension was stirred for 30 min. The precipitate was collected by filtration, washed with 20 mL ethanol and dried in vacuo at 50° C. to give a white solid. ¹H-NMR (400 MHz, DMSO-d₆) δ [ppm]: 1.035 (0.40), 1.052 (0.86), 1.070 (0.44), 1.171 (6.96), 1.189 (16.00), 1.206 (7.26), 2.268 (0.61), 2.285 (1.96), 2.304 (2.78), 2.322 (2.35), 2.331 (0.53), 2.340 (0.67), 2.518 (1.09), 2.522 (0.70), 3.820 (5.91), 3.838 (9.81), 3.856 (5.63), 3.976 (1.96), 3.994 (6.58), 4.011 (6.47), 4.028 (1.92), 6.191 (2.28), 6.195 (2.40), 6.221 (2.27), 6.225 (2.39), 6.335 (3.42), 6.337 (4.27), 6.340 (3.14), 6.905 (1.53), 6.908 (1.67), 6.924 (2.77), 6.926 (2.90), 6.942 (1.65), 6.945 (1.74), 6.972 (2.59), 6.974 (2.59), 6.989 (7.52), 6.991 (8.55), 7.320 (1.57), 7.325 (1.70), 7.339 (1.76), 7.341 (1.92), 7.343 (1.88), 7.345 (1.78), 7.359 (1.34), 7.363 (1.29), 7.757 (2.90), 7.762 (2.92), 7.777 (2.95), 7.781 (2.73); LC-MS (method 1): Rₜ=1.26 min; MS (ESIpos): m/z=419 [M+2H]⁺

### Example 127

### 6-(Azetidin-1-yl)-N-(5-tert-butyl-2-methoxybenzene-1-sulfonyl)-4-fluoro-1-benzofuran-2-carboxamide

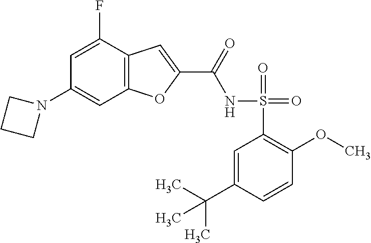 [SMILES: COc1ccc(C(C)(C)C)cc1S(=O)(=O)NC(=O)c1cc2c(F)cc(N3CCC3)cc2o1]

### 6-(Azetidin-1-yl)-4-fluoro-1-benzofuran-2-carboxylic acid

Intermediate 3, 85.0 mg, 361 μmol) and CDI (70.3 mg, 434 μmol; CAS-RN:[530-62-1]) was dissolved in THF (2.0 ml) and stirred at room temperature. After 1 h, commercially available 5-tert-butyl-2-methoxybenzene-1-sulfonamide (CAS: 88085-79-4, 132 mg, 542 μmol) was added followed by DBU (81 μL, 540 μmol; CAS-RN:[6674-22-2]) and the resulting solution was stirred at room temperature overnight. The reaction mixture was concentrated in vacuo, the residue was diluted with 2 mL DMSO and purified by preparative HPLC (water+0.1% HCOOH/acetonitrile) to give 109 mg (62% yield) of the title compound as an offwhite solid. ¹H-NMR (400 MHz, DMSO-d₆) δ [ppm]: 1.300 (16.00), 2.083 (0.99), 2.322 (0.52), 3.835 (4.57), 3.869 (1.02), 3.887 (1.68), 3.906 (0.96), 6.313 (0.47), 6.339 (1.21), 7.145 (0.70), 7.167 (0.75), 7.691 (0.42), 7.853 (1.09), 7.860 (1.03), 8.020 (0.44); LC-MS (method 1): Rₜ=1.41 min; MS (ESIpos): m/z=461 [M+H]⁺

### Example 128

### 6-(Azetidin-1-yl)-N-[2-ethoxy-5-(propan-2-yl)benzene-1-sulfonyl]-4-fluoro-1-benzofuran-2-carboxamide

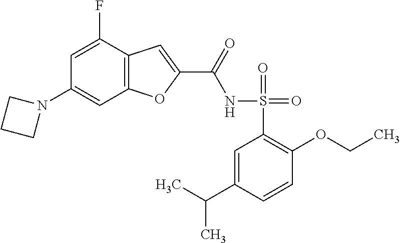 [SMILES: CCOc1ccc(C(C)C)cc1S(=O)(=O)NC(=O)c1cc2c(F)cc(N3CCC3)cc2o1]

Synthesized and purified analogously to Example 127 using commercially available 2-ethoxy-5-(propan-2-yl)benzene-1-sulfonamide (CAS: 1048919-96-5, 102 mg, 421 μmol) instead of 5-tert-butyl-2-methoxybenzene-1-sulfonamide to give 77.4 mg (43% yield) of the title compound as a white solid. ¹H-NMR (400 MHz, DMSO-d₆) δ [ppm]: 0.000 (7.43), 1.205 (15.80), 1.210 (5.66), 1.222 (16.00), 1.228 (9.69), 1.245 (3.93), 2.087 (1.42), 2.308 (1.10), 2.326 (1.75), 2.345 (1.20), 2.521 (1.19), 2.526 (0.77), 2.940 (0.81), 2.957 (1.07), 2.974 (0.78), 3.874 (3.23), 3.892 (5.41), 3.911 (3.09), 4.097 (0.89), 4.115 (2.82), 4.132 (2.82), 4.150 (0.91), 6.316 (1.14), 6.320 (1.38), 6.346 (5.39), 7.130 (1.52), 7.151 (1.68), 7.509 (0.87), 7.514 (0.92), 7.531 (0.78), 7.536 (0.78), 7.725 (2.68), 7.731 (2.49), 8.041 (0.66), 12.385 (0.66); LC-MS (method 1): Rₜ=1.42 min; MS (ESIpos): m/z=461 [M+H]⁺

### Example 129

### 6-(Azetidin-1-yl)-4-fluoro-N-{2-[(propan-2-yl)oxy]benzene-1-sulfonyl}-1-benzofuran-2-carboxamide

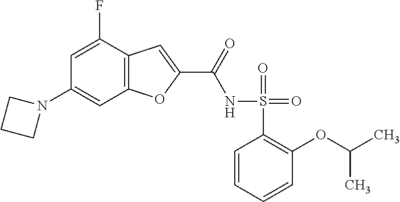 [SMILES: CC(C)Oc1ccccc1S(=O)(=O)NC(=O)c1cc2c(F)cc(N3CCC3)cc2o1]

Synthesized and purified analogously to Example 127 using commercially available 2-[(propan-2-yl)oxy]benzene-1-sulfonamide (CAS: 1517704-54-9, 90.6 mg, 421 μmol) instead of 5-tert-butyl-2-methoxybenzene-1-sulfonamide to give 40.1 mg (24% yield) of the title compound as an offwhite solid. ¹H-NMR (400 MHz, DMSO-d₆) δ [ppm]: 0.000 (7.75), 1.177 (15.78), 1.192 (16.00), 2.087 (2.13), 2.306 (1.20), 2.325 (1.90), 2.344 (1.30), 2.362 (0.41), 2.521 (1.09), 2.526 (0.73), 3.873 (3.46), 3.892 (5.78), 3.910 (3.31), 4.789 (0.73), 4.804 (0.98), 4.819 (0.74), 6.317 (1.20), 6.320 (1.37), 6.352 (3.31), 6.354 (3.28), 7.082 (0.85), 7.101 (1.63), 7.120 (0.90), 7.224 (1.30), 7.245 (1.42), 7.605 (0.59), 7.622 (1.00), 7.640 (0.49), 7.886 (1.60), 7.891 (1.59), 7.906 (1.53), 7.911 (1.42), 8.062 (0.76), 12.333 (0.78); LC-MS (method 1): Rₜ=1.30 min; MS (ESIpos): m/z=433 [M+H]⁺

### Example 130

### 6-(Azetidin-1-yl)-4-fluoro-N-(2-methylquinoline-8-sulfonyl)-1-benzofuran-2-carboxamide

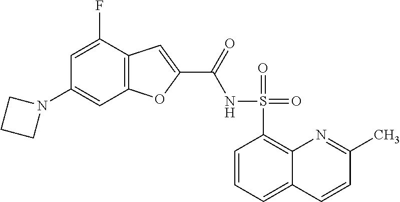 [SMILES: Cc1ccc2cccc(S(=O)(=O)NC(=O)c3cc4c(F)cc(N5CCC5)cc4o3)c2n1]

Synthesized and purified analogously to Example 127 using commercially available 2-methylquinoline-8-sulfonamide (CAS: 157686-27-6, 93.5 mg, 421 μmol) instead of 5-tert-butyl-2-methoxybenzene-1-sulfonamide to give 91.2 mg (52% yield) of the title compound as an offwhite solid. ¹H-NMR (400 MHz, DMSO-d₆) δ [ppm]: 0.000 (9.79), 2.079 (0.44), 2.087 (0.60), 2.272 (0.48), 2.289 (1.47), 2.308 (2.13), 2.327 (1.91), 2.336 (0.56), 2.344 (0.54), 2.523 (1.37), 2.527 (0.90), 2.684 (16.00), 3.365 (0.55), 3.847 (4.45), 3.866 (7.35), 3.884 (4.38), 6.300 (6.76), 6.327 (1.67), 6.331 (1.37), 7.510 (2.64), 7.531 (2.73), 7.724 (0.99), 7.743 (1.82), 7.763 (1.08), 8.277 (1.08), 8.297 (0.99), 8.380 (2.39), 8.402 (2.26), 8.443 (1.58), 8.446 (1.59), 8.462 (1.51), 8.465 (1.43); LC-MS (method 1): Rₜ=1.18 min; MS (ESIpos): m/z=440 [M+H]⁺

### Example 131

### 6-(Azetidin-1-yl)-N-[2-(cyclopropyloxy)benzene-1-sulfonyl]-4-fluoro-1-benzofuran-2-carboxamide

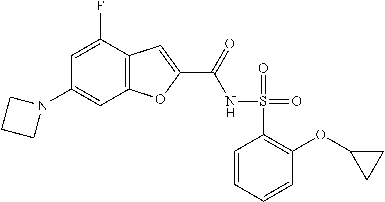 [SMILES: O=C(NS(=O)(=O)c1ccccc1OC1CC1)c1cc2c(F)cc(N3CCC3)cc2o1]

Synthesized and purified analogously to Example 127 using commercially available 2-(cyclopropyloxy)benzene-1-sulfonamide (CAS: 1243451-33-3, 89.8 mg, 421 μmol) instead of 5-tert-butyl-2-methoxybenzene-1-sulfonamide to give 88.7 mg (53% yield) of the title compound as an offwhite solid. ¹H-NMR (400 MHz, DMSO-d₆) δ [ppm]: −0.008 (0.44), 0.000 (11.40), 0.008 (0.41), 0.511 (1.35), 0.526 (4.69), 0.531 (7.34), 0.549 (1.83), 0.709 (1.48), 0.723 (5.31), 0.742 (4.93), 0.756 (1.15), 1.233 (0.41), 2.087 (6.13), 2.291 (1.06), 2.308 (3.35), 2.327 (5.09), 2.346 (3.64), 2.364 (1.14), 2.526 (1.50), 2.672 (0.49), 3.876 (9.45), 3.895 (16.00), 3.913 (8.99), 4.059 (1.45), 4.067 (2.08), 4.074 (2.72), 4.081 (2.04), 4.089 (1.44), 6.319 (3.10), 6.323 (3.63), 6.352 (13.68), 7.155 (1.85), 7.174 (3.68), 7.193 (2.06), 7.458 (3.26), 7.479 (3.89), 7.666 (1.50), 7.685 (2.42), 7.704 (1.16), 7.879 (3.70), 7.883 (3.75), 7.899 (3.62), 7.902 (3.43), 8.001 (1.08), 12.400 (1.20); LC-MS (method 1): Rₜ=1.26 min; MS (ESIpos): m/z=431 [M+H]⁺

### Example 132

### 6-(Azetidin-1-yl)-N-(2-chloroquinoline-8-sulfonyl)-4-fluoro-1-benzofuran-2-carboxamide

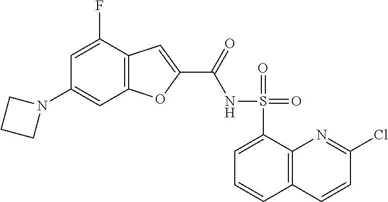 [SMILES: O=C(NS(=O)(=O)c1cccc2ccc(Cl)nc12)c1cc2c(F)cc(N3CCC3)cc2o1]

Synthesized and purified analogously to Example 127 using 2-chloroquinoline-8-sulfonamide (102 mg, 421 μmol) instead of 5-tert-butyl-2-methoxybenzene-1-sulfonamide to give 37.5 mg (21% yield) of the title compound as a yellow solid. ¹H-NMR (400 MHz, DMSO-d₆) δ [ppm]: −0.008 (0.48), 0.000 (12.82), 0.008 (0.45), 1.230 (0.47), 2.089 (14.05), 2.273 (1.03), 2.291 (3.19), 2.310 (4.53), 2.328 (3.89), 2.346 (1.12), 2.528 (1.73), 2.670 (0.45), 2.674 (0.64), 2.679 (0.44), 3.824 (0.63), 3.853 (9.10), 3.871 (15.26), 3.889 (8.68), 6.305 (16.00), 6.332 (3.83), 6.336 (3.20), 7.725 (7.39), 7.746 (7.68), 7.865 (3.44), 7.885 (5.60), 7.904 (3.74), 8.148 (2.32), 8.414 (3.63), 8.432 (3.28), 8.434 (3.23), 8.560 (4.21), 8.564 (4.31), 8.579 (4.16), 8.582 (3.87), 8.598 (7.25), 8.620 (6.99), 12.898 (0.41); LC-MS (method 1): Rₜ=1.29 min; MS (ESIpos): m/z=460 [M+H]⁺

### Example 133

### 6-(Azetidin-1-yl)-4-fluoro-N-(2-propoxybenzene-1-sulfonyl)-1-benzofuran-2-carboxamide

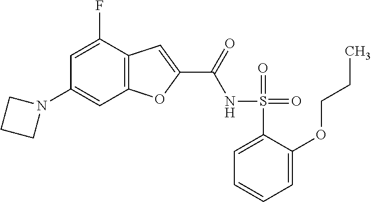 [SMILES: CCCOc1ccccc1S(=O)(=O)NC(=O)c1cc2c(F)cc(N3CCC3)cc2o1]

Synthesized and purified analogously to Example 127 using commercially available 2-propoxybenzene-1-sulfonamide (CAS: 196107-68-3, 137 mg, 638 μmol) instead of 5-tert-butyl-2-methoxybenzene-1-sulfonamide to give 26.0 mg (13% yield) of the title compound as an offwhite solid. ¹H-NMR (400 MHz, DMSO-d₆) δ [ppm]: 0.858 (6.96), 0.876 (16.00), 0.894 (7.46), 1.021 (0.44), 1.039 (0.72), 1.231 (0.75), 1.658 (0.51), 1.676 (1.85), 1.693 (3.69), 1.711 (3.50), 1.727 (1.71), 1.745 (0.45), 2.286 (0.68), 2.304 (2.07), 2.323 (3.42), 2.342 (2.21), 2.359 (0.70), 2.518 (3.18), 2.522 (2.00), 2.664 (0.59), 2.669 (0.80), 2.673 (0.59), 3.870 (5.89), 3.889 (9.80), 3.907 (5.56), 4.040 (2.85), 4.056 (5.74), 4.072 (2.78), 5.758 (6.80), 6.310 (1.94), 6.314 (2.28), 6.344 (7.34), 7.102 (1.25), 7.121 (2.49), 7.140 (1.38), 7.200 (2.03), 7.221 (2.23), 7.616 (0.93), 7.633 (1.65), 7.653 (0.89), 7.892 (2.46), 7.897 (2.54), 7.913 (2.39), 7.916 (2.31), 8.011 (0.88), 12.410 (0.70); LC-MS (method 1): Rₜ=1.35 min; MS (ESIpos): m/z=433 [M+H]⁺

### Example 134

### 6-(Azetidin-1-yl)-4-fluoro-N-{5-methyl-2-[(propan-2-yl)oxy]benzene-1-sulfonyl}-1-benzofuran-2-carboxamide

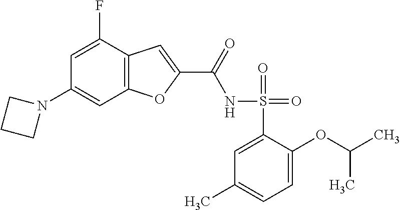 [SMILES: Cc1ccc(OC(C)C)c(S(=O)(=O)NC(=O)c2cc3c(F)cc(N4CCC4)cc3o2)c1]

Synthesized analogously to Example 127 using commercially available 2-isopropoxy-5-methyl-benzenesulfonamide (CAS: 1094691-36-7, 137 mg, 638 μmol) instead of 5-tert-butyl-2-methoxybenzene-1-sulfonamide to give 32.0 mg (16% yield) of the title compound as an offwhite solid. ¹H-NMR (400 MHz, DMSO-d₆) δ [ppm]: 1.140 (15.75), 1.156 (16.00), 1.230 (0.56), 2.074 (0.95), 2.084 (3.46), 2.284 (0.43), 2.308 (10.86), 2.320 (2.11), 2.332 (0.85), 2.338 (1.34), 2.356 (0.41), 2.518 (1.82), 2.522 (1.16), 2.539 (1.83), 2.669 (0.42), 3.865 (3.35), 3.883 (5.65), 3.901 (3.21), 4.700 (0.66), 4.715 (0.88), 4.730 (0.67), 5.758 (0.40), 6.297 (1.16), 6.300 (1.30), 6.326 (1.10), 6.330 (1.40), 6.349 (2.80), 7.081 (1.18), 7.102 (1.35), 7.373 (0.83), 7.391 (0.73), 7.675 (2.02), 7.679 (1.99), 8.133 (0.45); LC-MS (method 1): Rₜ=1.39 min; MS (ESIpos): m/z=447 [M+H]⁺

### Example 135

### 6-(Azetidin-1-yl)-N-(5-bromo-2-ethoxybenzene-1-sulfonyl)-4-fluoro-1-benzofuran-2-carboxamide

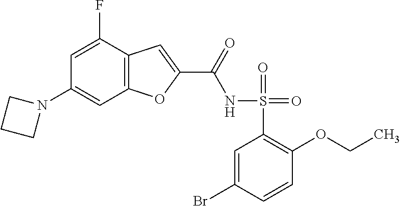 [SMILES: CCOc1ccc(Br)cc1S(=O)(=O)NC(=O)c1cc2c(F)cc(N3CCC3)cc2o1]

Synthesized and purified analogously to Example 127 using commercially available 5-bromo-2-ethoxybenzene-1-sulfonamide (CAS: 327081-38-9, 179 mg, 638 μmol) instead of 5-tert-butyl-2-methoxybenzene-1-sulfonamide to give 9.40 mg (4% yield) of the title compound as an offwhite solid. ¹H-NMR (400 MHz, DMSO-d₆) δ [ppm]: 1.199 (7.35), 1.216 (16.00), 1.233 (7.56), 1.362 (0.47), 1.620 (0.43), 1.912 (0.49), 2.284 (0.68), 2.302 (2.15), 2.321 (3.25), 2.332 (1.18), 2.339 (2.38), 2.358 (0.76), 2.518 (1.46), 2.523 (1.04), 2.646 (0.41), 2.669 (0.46), 3.249 (0.52), 3.459 (0.43), 3.473 (0.67), 3.537 (0.45), 3.561 (0.46), 3.864 (6.35), 3.882 (10.62), 3.901 (6.15), 4.108 (1.35), 4.125 (3.95), 4.143 (3.90), 4.160 (1.30), 5.758 (1.59), 6.292 (2.04), 6.296 (2.25), 6.322 (1.95), 6.326 (2.35), 6.345 (4.58), 6.347 (5.43), 6.351 (3.67), 7.157 (1.82), 7.180 (1.95), 7.674 (1.59), 7.677 (1.62), 7.754 (1.20), 7.775 (1.15), 7.788 (0.62), 7.794 (0.48), 7.919 (6.28), 7.926 (5.78), 9.029 (0.58); LC-MS (method 1): Rₜ=1.38 min; MS (ESIpos): m/z=495 [M+H]⁺

### Example 136

### 6-(azetidin-1-yl)-N-(2-ethyl-6-methoxybenzene-1-sulfonyl)-4-fluoro-1-benzofuran-2-carboxamide

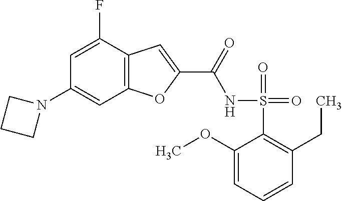 [SMILES: CCc1cccc(OC)c1S(=O)(=O)NC(=O)c1cc2c(F)cc(N3CCC3)cc2o1]

Synthesized and purified analogously to Example 127 using 2-ethyl-6-methoxybenzene-1-sulfonamide (137 mg, 638 μmol) instead of 5-tert-butyl-2-methoxybenzene-1-sulfonamide to give 27.6 mg (14% yield) of the title compound as a yellow solid. ¹H-NMR (400 MHz, DMSO-d₆) δ [ppm]: 1.214 (2.15), 1.232 (5.24), 1.251 (2.21), 2.083 (0.79), 2.305 (0.81), 2.323 (1.36), 2.342 (0.89), 2.518 (1.42), 2.522 (0.94), 2.539 (16.00), 3.085 (0.57), 3.104 (1.81), 3.122 (1.78), 3.141 (0.52), 3.806 (9.70), 3.871 (2.40), 3.889 (3.99), 3.907 (2.27), 5.758 (0.88), 6.310 (0.83), 6.314 (1.01), 6.342 (4.26), 6.967 (1.04), 6.985 (1.08), 7.034 (0.97), 7.054 (1.08), 7.481 (0.70), 7.501 (1.11), 7.521 (0.60), 8.009 (0.41), LC-MS (method 1): Rₜ=1.35 min; MS (ESIpos): m/z=433 [M+H]⁺

### Example 137

### 6-(Azetidin-1-yl)-4-fluoro-N-[2-methoxy-5-(morpholin-4-yl)benzene-1-sulfonyl]-1-benzofuran-2-carboxamide

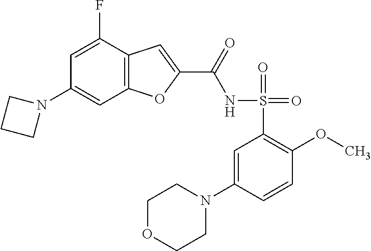 [SMILES: COc1ccc(N2CCOCC2)cc1S(=O)(=O)NC(=O)c1cc2c(F)cc(N3CCC3)cc2o1]

Synthesized and purified analogously to Example 127 using 2-methoxy-5-(morpholin-4-yl)benzene-1-sulfonamide (53.0 mg, 195 μmol) instead of 5-tert-butyl-2-methoxybenzene-1-sulfonamide to give 50.0 mg (62% yield) of the title compound as a yellow solid. ¹H-NMR (400 MHz, DMSO-d₆) δ [ppm]: 2.083 (1.91), 2.288 (0.50), 2.305 (1.60), 2.323 (2.62), 2.342 (1.72), 2.359 (0.55), 2.669 (0.65), 3.047 (4.05), 3.059 (5.39), 3.070 (4.49), 3.310 (0.99), 3.390 (1.20), 3.395 (1.08), 3.744 (4.67), 3.756 (5.58), 3.767 (4.69), 3.783 (16.00), 3.871 (4.45), 3.889 (7.56), 3.908 (4.28), 6.314 (1.88), 6.347 (4.60), 7.122 (1.71), 7.144 (2.32), 7.265 (1.13), 7.287 (0.86), 7.396 (3.66), 7.404 (3.32), 8.014 (0.82), 12.496 (0.47); LC-MS (method 1): Rₜ=1.20 min; MS (ESIneg): m/z=488 [M−H]⁻

### Example 138

### 6-(Azetidin-1-yl)-4-fluoro-N-[2-(trifluoromethoxy)benzene-1-sulfonyl]-1-benzofuran-2-carboxamide

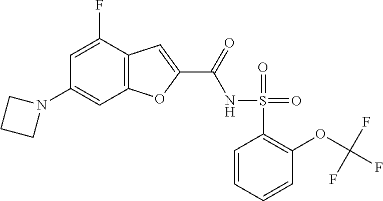 [SMILES: O=C(NS(=O)(=O)c1ccccc1OC(F)(F)F)c1cc2c(F)cc(N3CCC3)cc2o1]

Synthesized analogously to Example 127 using commercially available 2-(trifluoromethoxy)benzene-1-sulfonamide (CAS: 37526-59-3, 154 mg, 638 μmol) instead of 5-tert-butyl-2-methoxybenzene-1-sulfonamide to give 9.30 mg (4% yield) of the title compound as an offwhite solid. ¹H-NMR (400 MHz, DMSO-d₆) δ [ppm]: 1.026 (0.67), 1.042 (0.72), 1.232 (1.12), 1.907 (1.57), 2.074 (1.06), 2.287 (1.24), 2.306 (3.58), 2.323 (7.25), 2.326 (7.70), 2.331 (4.15), 2.336 (2.64), 2.342 (3.72), 2.361 (1.20), 2.518 (14.05), 2.522 (8.84), 2.665 (2.83), 2.669 (3.88), 2.673 (2.77), 3.804 (0.69), 3.823 (1.06), 3.841 (0.78), 3.872 (9.61), 3.890 (16.00), 3.908 (9.04), 5.759 (11.89), 6.309 (3.03), 6.312 (3.36), 6.342 (5.12), 6.347 (8.64), 7.558 (1.87), 7.578 (2.18), 7.593 (1.79), 7.612 (3.20), 7.632 (1.90), 7.741 (0.45), 7.747 (0.45), 7.796 (1.48), 7.815 (2.29), 7.833 (1.41), 8.113 (3.17), 8.116 (3.09), 8.132 (2.99); LC-MS (method 1): Rₜ=1.31 min; MS (ESIpos): m/z=459 [M+H]⁺

### Example 139

### 6-(Azetidin-1-yl)-N-(5-bromo-2-methoxybenzene-1-sulfonyl)-4-fluoro-1-benzofuran-2-carboxamide

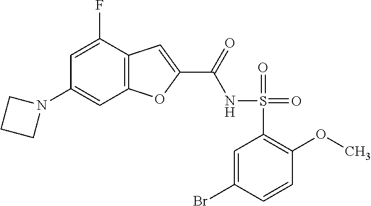 [SMILES: COc1ccc(Br)cc1S(=O)(=O)NC(=O)c1cc2c(F)cc(N3CCC3)cc2o1]

Synthesized and purified analogously to Example 127 using commercially available 5-bromo-2-methoxybenzene-1-sulfonamide (CAS: 23095-14-9, 136 mg, 510 μmol) instead of 5-tert-butyl-2-methoxybenzene-1-sulfonamide to give 137 mg (65% yield) of the title compound as yellow solid. ¹H-NMR (400 MHz, DMSO-d₆) δ [ppm]: 2.074 (0.82), 2.083 (10.54), 2.289 (0.42), 2.308 (1.35), 2.326 (2.31), 2.344 (1.59), 2.362 (0.57), 2.669 (0.42), 3.645 (0.48), 3.870 (16.00), 3.893 (6.85), 3.911 (3.97), 6.315 (1.34), 6.319 (1.64), 6.348 (4.51), 7.218 (2.23), 7.241 (2.40), 7.849 (1.20), 7.855 (1.40), 7.871 (1.15), 7.877 (1.34), 7.940 (4.02), 7.947 (3.62), 7.998 (1.16); LC-MS (method 1): Rₜ=1.31 min; MS (ESIpos): m/z=483 [M+H]⁺

### Example 140

### 6-(Azetidin-1-yl)-4-fluoro-N-(2,2,3,3-tetrafluoro-2,3-dihydro-1,4-benzodioxine-5-sulfonyl)-1-benzofuran-2-carboxamide

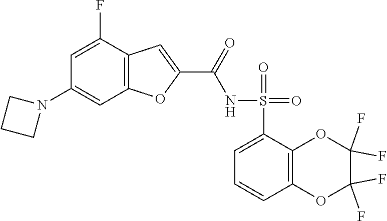 [SMILES: O=C(NS(=O)(=O)c1cccc2c1OC(F)(F)C(F)(F)O2)c1cc2c(F)cc(N3CCC3)cc2o1]

Synthesized and purified analogously to Example 127 using 2,2,3,3-tetrafluoro-2,3-dihydro-1,4-benzodioxine-5-sulfonamide (147 mg, 510 μmol) instead of 5-tert-butyl-2-methoxybenzene-1-sulfonamide to give 23.4 mg (10% yield) of the title compound as a yellow solid. ¹H-NMR (400 MHz, DMSO-d₆) δ [ppm]: 1.229 (0.61), 1.907 (0.53), 2.074 (2.18), 2.083 (6.10), 2.289 (1.06), 2.307 (3.23), 2.326 (5.48), 2.336 (1.60), 2.344 (3.50), 2.362 (1.15), 2.373 (1.12), 2.518 (3.95), 2.522 (2.56), 2.539 (3.85), 2.664 (0.87), 2.669 (1.20), 2.673 (0.87), 3.709 (0.51), 3.821 (0.44), 3.875 (9.85), 3.893 (16.00), 3.911 (9.36), 4.148 (0.51), 5.758 (1.09), 6.315 (3.46), 6.319 (4.05), 6.349 (9.95), 6.351 (9.08), 7.562 (3.38), 7.583 (6.56), 7.603 (4.19), 7.820 (3.64), 7.823 (3.83), 7.841 (3.24), 7.845 (3.14), 7.885 (2.79), 7.934 (4.61), 7.937 (4.49), 7.954 (4.26), 7.957 (3.85); LC-MS (method 1): Rₜ=1.33 min; MS (ESIpos): m/z=505 [M+H]⁺

### Example 141

### 6-(Azetidin-1-yl)-N-(1,1-dioxo-1,2,3,4-tetrahydro-1-benzothiopyran-8-sulfonyl)-4-fluoro-1-benzofuran-2-carboxamide

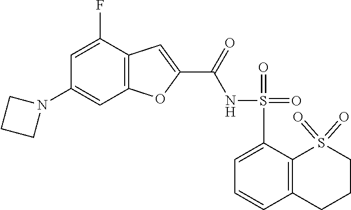 [SMILES: O=C(NS(=O)(=O)c1cccc2c1S(=O)(=O)CCC2)c1cc2c(F)cc(N3CCC3)cc2o1]

Synthesized and purified analogously to Example 127 using commercially available 1,1-dioxo-1,2,3,4-tetrahydro-1-benzothiopyran-8-sulfonamide (CAS: 87254-74-8, 142 mg, 542 μmol) instead of 5-tert-butyl-2-methoxybenzene-1-sulfonamide to give 31.0 mg (16% yield) of the title compound as an offwhite solid. ¹H-NMR (400 MHz, DMSO-d₆) δ [ppm]: 2.084 (16.00), 2.245 (0.61), 2.261 (1.77), 2.269 (1.64), 2.276 (2.24), 2.290 (2.36), 2.307 (2.74), 2.326 (3.81), 2.336 (1.20), 2.344 (2.24), 2.362 (0.71), 2.518 (3.79), 2.522 (2.37), 2.664 (0.75), 2.669 (1.03), 2.673 (0.75), 3.122 (2.17), 3.138 (3.97), 3.154 (2.02), 3.504 (2.25), 3.511 (2.02), 3.519 (2.23), 3.526 (1.98), 3.533 (2.10), 3.874 (6.11), 3.893 (10.04), 3.911 (5.80), 6.312 (2.14), 6.316 (2.48), 6.341 (1.88), 6.345 (3.04), 6.352 (4.78), 6.354 (5.39), 7.702 (1.13), 7.721 (1.95), 7.759 (1.67), 7.778 (2.45), 7.798 (1.02), 8.014 (0.72), 8.223 (2.36), 8.240 (2.19); LC-MS (method 1): Rₜ=1.18 min; MS (ESIpos): m/z=479 [M+H]⁺

### Example 142

### 6-(Azetidin-1-yl)-N-(5-chloro-2,2-difluoro-2H-1,3-benzodioxole-4-sulfonyl)-4-fluoro-1-benzofuran-2-carboxamide

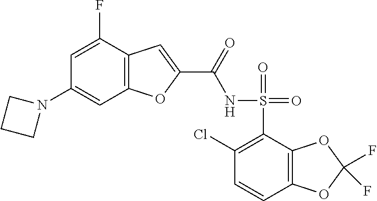 [SMILES: O=C(NS(=O)(=O)c1c(Cl)ccc2c1OC(F)(F)O2)c1cc2c(F)cc(N3CCC3)cc2o1]

Synthesized and purified analogously to Example 127 using 5-chloro-2,2-difluoro-2H-1,3-benzodioxole-4-sulfonamide (147 mg, 542 μmol) instead of 5-tert-butyl-2-methoxybenzene-1-sulfonamide to give 118 mg (60% yield) of the title compound as an offwhite solid. ¹H-NMR (400 MHz, DMSO-d₆) δ [ppm]: 2.083 (13.68), 2.290 (0.96), 2.308 (3.06), 2.326 (5.31), 2.345 (3.36), 2.363 (1.07), 2.518 (3.65), 2.522 (2.39), 2.664 (0.70), 2.669 (0.99), 2.673 (0.74), 3.876 (9.89), 3.894 (16.00), 3.913 (9.53), 6.306 (3.40), 6.309 (4.16), 6.339 (12.44), 7.466 (7.21), 7.488 (8.64), 7.692 (7.66), 7.713 (6.37), 7.799 (3.11), LC-MS (method 1): Rₜ=1.23 min; MS (ESIpos): m/z=489 [M+H]⁺

### Example 143

### 6-(Azetidin-1-yl)-N-(1-ethyl-1H-pyrazolo[3,4-b]pyridine-3-sulfonyl)-4-fluoro-1-benzofuran-2-carboxamide

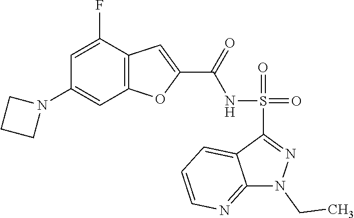 [SMILES: CCn1nc(S(=O)(=O)NC(=O)c2cc3c(F)cc(N4CCC4)cc3o2)c2cccnc21]

Synthesized and purified analogously to Example 127 using 1-ethyl-1H-pyrazolo[3,4-b]pyridine-3-sulfonamide (123 mg, 542 μmol) instead of 5-tert-butyl-2-methoxybenzene-1-sulfonamide to give the title compound as an offwhite solid. LC-MS (method 1): Rₜ=1.18 min; MS (ESIpos): m/z=444 [M+H]⁺ ¹H-NMR (400 MHz, DMSO-d₆) δ [ppm]: 1.451 (7.34), 1.468 (16.00), 1.487 (7.52), 2.082 (4.37), 2.278 (0.73), 2.296 (2.33), 2.315 (3.41), 2.326 (1.48), 2.333 (2.68), 2.351 (0.83), 2.836 (0.93), 3.865 (6.68), 3.883 (11.31), 3.894 (2.59), 3.901 (6.47), 3.912 (0.81), 4.126 (1.37), 4.590 (2.07), 4.608 (6.71), 4.627 (6.60), 4.644 (2.03), 6.306 (10.30), 6.332 (2.98), 6.336 (2.33), 6.349 (0.58), 7.512 (3.05), 7.523 (3.17), 7.533 (3.16), 7.544 (3.05), 7.903 (5.89), 8.490 (3.58), 8.494 (3.81), 8.510 (3.34), 8.515 (3.32), 8.733 (3.58), 8.737 (3.67), 8.744 (3.52), 8.748 (3.24).

### Example 144

### 6-(Azetidin-1-yl)-4-fluoro-N-[1-methyl-6-(trifluoromethyl)-1H-pyrazolo[3,4-b]pyridine-3-sulfonyl]-1-benzofuran-2-carboxamide

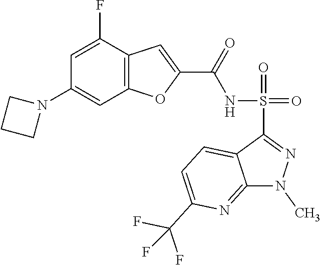 [SMILES: Cn1nc(S(=O)(=O)NC(=O)c2cc3c(F)cc(N4CCC4)cc3o2)c2ccc(C(F)(F)F)nc21]

Synthesized and purified analogously to Example 127 using 1-methyl-6-(trifluoromethyl)-1H-pyrazolo[3,4-b]pyridine-3-sulfonamide (152 mg, 542 μmol) instead of 5-tert-butyl-2-methoxybenzene-1-sulfonamide to give 117 mg (62% yield) of the title compound as an offwhite solid. ¹H-NMR (400 MHz, DMSO-d₆) δ [ppm]: 2.074 (5.63), 2.083 (2.07), 2.299 (1.08), 2.318 (1.62), 2.326 (0.69), 2.336 (1.25), 2.518 (0.95), 2.522 (0.59), 3.865 (3.31), 3.884 (5.38), 3.902 (3.14), 4.225 (16.00), 6.302 (5.80), 6.328 (1.50), 6.332 (1.16), 7.837 (2.06), 7.995 (3.33), 8.016 (3.18), 8.787 (2.16), 8.808 (1.94); LC-MS (method 2): Rₜ=0.84 min; MS (ESIpos): m/z=498 [M+H]⁺

### Example 145

### 6-(Azetidin-1-yl)-N-(1,6-dimethyl-1H-pyrazolo[3,4-b]pyridine-3-sulfonyl)-4-fluoro-1-benzofuran-2-carboxamide

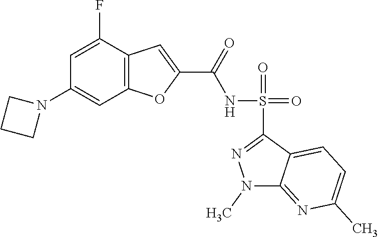 [SMILES: Cc1ccc2c(S(=O)(=O)NC(=O)c3cc4c(F)cc(N5CCC5)cc4o3)nn(C)c2n1]

Synthesized and purified analogously to Example 127 using 1,6-dimethyl-1H-pyrazolo[3,4-b]pyridine-3-sulfonamide (123 mg, 542 μmol) instead of 5-tert-butyl-2-methoxybenzene-1-sulfonamide to give 99.8 mg (59% yield) of the title compound as an offwhite solid. ¹H-NMR (400 MHz, DMSO-d₆) δ [ppm]: 2.083 (2.78), 2.298 (1.26), 2.316 (1.85), 2.335 (1.43), 2.352 (0.44), 2.667 (14.44), 3.866 (3.57), 3.884 (6.06), 3.902 (3.45), 4.128 (16.00), 6.306 (4.44), 6.332 (1.63), 6.335 (1.30), 7.388 (2.85), 7.409 (2.98), 7.901 (3.05), 8.333 (3.26), 8.354 (2.99); LC-MS (method 1): Rₜ=1.17 min; MS (ESIneg): m/z=442 [M−H]⁻

### Example 146

### 6-(Azetidin-1-yl)-N-(1,4-dimethyl-1H-pyrazolo[3,4-b]pyridine-3-sulfonyl)-4-fluoro-1-benzofuran-2-carboxamide

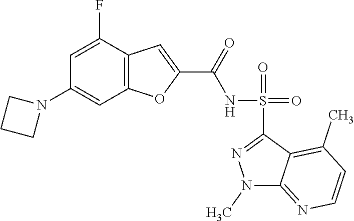 [SMILES: Cc1ccnc2c1c(S(=O)(=O)NC(=O)c1cc3c(F)cc(N4CCC4)cc3o1)nn2C]

Synthesized and purified analogously to Example 127 using 1,4-dimethyl-1H-pyrazolo[3,4-b]pyridine-3-sulfonamide (123 mg, 542 μmol) instead of 5-tert-butyl-2-methoxybenzene-1-sulfonamide to give 69.0 mg (41% yield) of the title compound as an offwhite solid. ¹H-NMR (400 MHz, DMSO-d₆) δ [ppm]: 2.074 (0.80), 2.082 (4.17), 2.306 (1.22), 2.324 (1.80), 2.342 (1.32), 2.360 (0.43), 2.835 (10.80), 3.875 (3.48), 3.893 (5.86), 3.911 (3.32), 4.127 (16.00), 6.316 (1.26), 6.320 (1.55), 6.347 (5.61), 7.284 (1.70), 7.286 (1.73), 7.298 (1.77), 7.968 (2.60), 8.557 (3.00), 8.568 (2.82); LC-MS (method 1): Rₜ=1.12 min; MS (ESIneg): m/z=442 [M−H]⁻

### Example 147

### 6-(Azetidin-1-yl)-N-(2-chloro-6-propylimidazo[1,2-b]pyridazine-3-sulfonyl)-4-fluoro-1-benzofuran-2-carboxamide

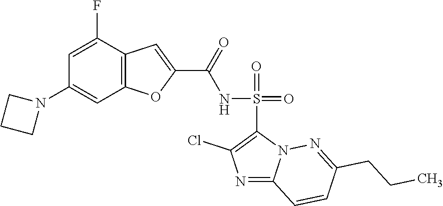 [SMILES: CCCc1ccc2nc(Cl)c(S(=O)(=O)NC(=O)c3cc4c(F)cc(N5CCC5)cc4o3)n2n1]

Synthesized and purified analogously to Example 127 using 2-chloro-6-propylimidazo[1,2-b]pyridazine-3-sulfonamide (149 mg, 542 μmol) instead of 5-tert-butyl-2-methoxybenzene-1-sulfonamide to give 8.90 mg (5% yield) of the title compound as an offwhite solid. ¹H-NMR (400 MHz, DMSO-d₆) δ [ppm]: 0.682 (6.32), 0.700 (14.52), 0.719 (6.91), 1.231 (0.63), 1.539 (0.48), 1.557 (1.97), 1.576 (3.42), 1.594 (3.45), 1.613 (1.90), 1.631 (0.47), 2.074 (1.47), 2.084 (16.00), 2.282 (0.74), 2.300 (2.18), 2.319 (3.47), 2.327 (2.26), 2.337 (2.76), 2.354 (0.81), 2.523 (5.10), 2.665 (0.90), 2.669 (1.23), 2.673 (0.89), 2.763 (3.31), 2.782 (4.62), 2.801 (3.12), 3.147 (0.51), 3.863 (6.13), 3.882 (10.28), 3.900 (5.96), 5.759 (0.90), 6.304 (3.17), 6.312 (5.28), 6.330 (2.75), 7.502 (3.32), 7.526 (3.54), 7.950 (0.98), 8.197 (4.11), 8.220 (3.79); LC-MS (method 1): Rₜ=1.24 min; MS (ESIpos): m/z=492 [M+H]⁺

### Example 148

### 6-(Azetidin-1-yl)-N-[2-(ethylsulfanyl)imidazo[1,2-a]pyridine-3-sulfonyl]-4-fluoro-1-benzofuran-2-carboxamide

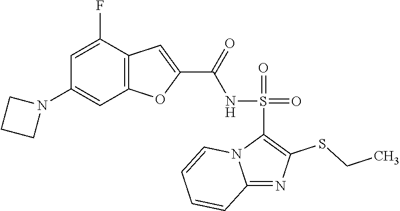 [SMILES: CCSc1nc2ccccn2c1S(=O)(=O)NC(=O)c1cc2c(F)cc(N3CCC3)cc2o1]

Synthesized and purified analogously to Example 127 using 2-(ethylsulfanyl)imidazo[1,2-a]pyridine-3-sulfonamide (139 mg, 542 μmol) instead of 5-tert-butyl-2-methoxybenzene-1-sulfonamide to give 22.3 mg (13% yield) of the title compound as an offwhite solid. ¹H-NMR (400 MHz, DMSO-d₆) δ [ppm]: 1.229 (0.74), 1.284 (6.91), 1.303 (16.00), 1.321 (7.28), 2.073 (6.47), 2.083 (3.38), 2.280 (0.58), 2.298 (1.79), 2.317 (2.80), 2.326 (1.54), 2.335 (2.23), 2.353 (0.71), 2.522 (2.87), 2.664 (0.53), 2.669 (0.70), 2.673 (0.49), 3.185 (2.08), 3.202 (6.18), 3.221 (6.24), 3.239 (2.37), 3.865 (5.16), 3.884 (8.71), 3.902 (5.09), 5.758 (0.52), 6.298 (7.52), 6.328 (2.24), 6.331 (1.90), 7.226 (1.07), 7.244 (2.20), 7.258 (1.22), 7.580 (1.00), 7.599 (1.73), 7.617 (1.28), 7.719 (2.98), 7.742 (2.16), 7.845 (1.38), 8.860 (3.02), 8.863 (1.91), 8.878 (2.83), LC-MS (method 1): Rₜ=1.27 min; MS (ESIpos): m/z=475 [M+H]⁺

### Example 149

### 6-(Azetidin-1-yl)-4-fluoro-N-[2-(methylsulfanyl)imidazo[1,2-a]pyridine-3-sulfonyl]-1-benzofuran-2-carboxamide

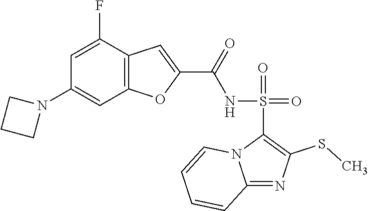 [SMILES: CSc1nc2ccccn2c1S(=O)(=O)NC(=O)c1cc2c(F)cc(N3CCC3)cc2o1]

Synthesized and purified analogously to Example 127 using 2-(methylsulfanyl)imidazo[1,2-a]pyridine-3-sulfonamide (132 mg, 542 μmol) instead of 5-tert-butyl-2-methoxybenzene-1-sulfonamide to give 16.5 mg (9% yield) of the title compound as an offwhite solid. ¹H-NMR (400 MHz, DMSO-d₆) δ [ppm]: 2.084 (1.02), 2.281 (0.40), 2.299 (1.24), 2.318 (1.96), 2.327 (1.07), 2.337 (1.55), 2.354 (0.46), 2.595 (16.00), 2.669 (0.51), 3.866 (3.54), 3.885 (6.02), 3.903 (3.44), 6.298 (5.41), 6.327 (1.53), 6.331 (1.30), 7.233 (0.73), 7.250 (1.47), 7.265 (0.83), 7.583 (0.71), 7.603 (1.18), 7.623 (0.86), 7.724 (1.96), 7.747 (1.44), 7.835 (0.90), 8.854 (1.97), 8.871 (1.92); LC-MS (method 1): Rₜ=1.20 min; MS (ESIpos): m/z=461 [M+H]⁺

### Example 150

### N-(5-tert-butyl-2-methoxybenzene-1-sulfonyl)-4-fluoro-6-(3-fluoroazetidin-1-yl)-1-benzofuran-2-carboxamide

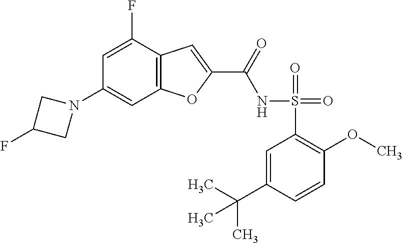 [SMILES: COc1ccc(C(C)(C)C)cc1S(=O)(=O)NC(=O)c1cc2c(F)cc(N3CC(F)C3)cc2o1]

Synthesized and purified analogously to Example 127 using 4-fluoro-6-(3-fluoroazetidin-1-yl)-1-benzofuran-2-carboxylic acid (Intermediate 18, 85 mg, 336 μmol) instead of 6-(azetidin-1-yl)-4-fluoro-1-benzofuran-2-carboxylic acid to give 42.7 mg (25% yield) of the title compound as an offwhite solid. ¹H-NMR (400 MHz, DMSO-d₆) δ [ppm]: 1.302 (16.00), 2.084 (3.75), 2.518 (0.74), 2.522 (0.48), 3.836 (3.94), 6.431 (0.40), 6.460 (0.46), 6.471 (0.72), 6.473 (0.82), 7.147 (0.52), 7.169 (0.56), 7.854 (1.04), 7.860 (0.97), LC-MS (method 1): Rₜ=1.37 min; MS (ESIpos): m/z=479 [M+H]⁺

### Example 151

### N-[2-ethoxy-5-(propan-2-yl)benzene-1-sulfonyl]-4-fluoro-6-(3-fluoroazetidin-1-yl)-1-benzofuran-2-carboxamide

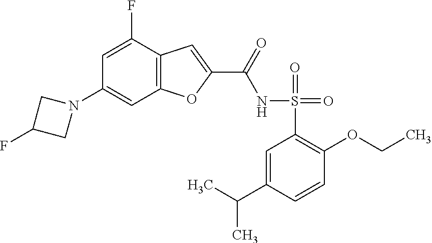 [SMILES: CCOc1ccc(C(C)C)cc1S(=O)(=O)NC(=O)c1cc2c(F)cc(N3CC(F)C3)cc2o1]

Synthesized and purified analogously to Example 150 using 2-ethoxy-5-(propan-2-yl)benzene-1-sulfonamide (106 mg, 434 μmol) instead of 5-tert-butyl-2-methoxybenzene-1-sulfonamide to give 85.0 mg (44% yield) of the title compound as an offwhite solid. ¹H-NMR (400 MHz, DMSO-d₆) δ [ppm]: 1.203 (15.09), 1.220 (16.00), 1.224 (11.96), 1.241 (4.14), 2.083 (4.54), 2.539 (2.40), 2.939 (0.79), 2.956 (1.05), 2.973 (0.79), 3.941 (0.61), 3.945 (0.68), 3.965 (0.82), 3.970 (0.83), 3.973 (0.82), 4.002 (0.67), 4.006 (0.72), 4.027 (0.81), 4.030 (0.80), 4.097 (0.90), 4.115 (2.94), 4.132 (2.96), 4.149 (0.99), 4.188 (0.64), 4.199 (0.76), 4.202 (0.78), 4.212 (0.67), 4.227 (0.64), 4.240 (0.70), 4.252 (0.76), 4.255 (0.76), 4.265 (0.67), 4.279 (0.58), 5.430 (0.49), 5.574 (0.49), 5.581 (0.42), 6.430 (1.09), 6.433 (1.33), 6.458 (1.01), 6.463 (1.47), 6.477 (2.65), 7.131 (1.68), 7.152 (1.90), 7.511 (0.95), 7.516 (1.05), 7.532 (0.90), 7.538 (0.95), 7.725 (2.53), 7.731 (2.51), 8.060 (0.90), 12.438 (0.45), LC-MS (method 1): Rₜ=1.38 min; MS (ESIpos): m/z=479 [M+H]⁺

### Example 152

### 4-Fluoro-6-(3-fluoroazetidin-1-yl)-N-(2-methylquinoline-8-sulfonyl)-1-benzofuran-2-carboxamide

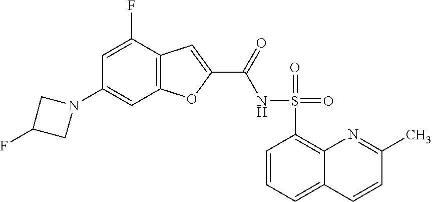 [SMILES: Cc1ccc2cccc(S(=O)(=O)NC(=O)c3cc4c(F)cc(N5CC(F)C5)cc4o3)c2n1]

Synthesized and purified analogously to Example 150 using 2-methylquinoline-8-sulfonamide (96.6 mg, 434 μmol) instead of 5-tert-butyl-2-methoxybenzene-1-sulfonamide to give 105 mg (57% yield) of the title compound as an offwhite solid. ¹H-NMR (400 MHz, DMSO-d₆) δ [ppm]: 2.073 (0.42), 2.083 (0.73), 2.327 (0.45), 2.539 (6.45), 2.665 (0.46), 2.669 (0.70), 2.681 (16.00), 3.915 (0.90), 3.918 (0.97), 3.939 (1.16), 3.943 (1.18), 3.946 (1.16), 3.971 (0.88), 3.975 (0.95), 3.979 (1.00), 4.000 (1.13), 4.004 (1.11), 4.162 (0.84), 4.174 (1.00), 4.177 (1.03), 4.187 (0.89), 4.201 (0.86), 4.215 (0.96), 4.226 (1.00), 4.229 (1.02), 4.237 (0.86), 4.240 (0.91), 4.254 (0.79), 5.406 (0.51), 5.414 (0.63), 5.421 (0.52), 5.550 (0.48), 5.557 (0.64), 5.564 (0.53), 5.572 (0.40), 6.412 (1.28), 6.416 (1.89), 6.434 (3.37), 6.442 (2.38), 6.446 (1.46), 7.511 (3.09), 7.533 (3.18), 7.728 (1.40), 7.747 (2.34), 7.766 (1.54), 8.146 (0.90), 8.284 (1.51), 8.302 (1.37), 8.304 (1.39), 8.383 (2.85), 8.404 (2.79), 8.446 (1.89), 8.450 (1.98), 8.465 (1.84), 8.469 (1.83), LC-MS (method 1): Rₜ=1.12 min; MS (ESIpos): m/z=458 [M+H]⁺

### Example 153

### N-(2-ethoxy-5-ethylbenzene-1-sulfonyl)-4-fluoro-6-(3-fluoroazetidin-1-yl)-1-benzofuran-2-carboxamide

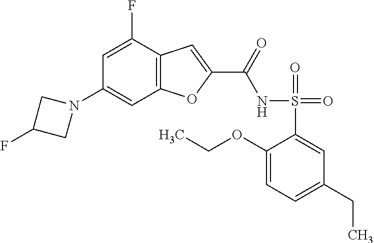 [SMILES: CCOc1ccc(CC)cc1S(=O)(=O)NC(=O)c1cc2c(F)cc(N3CC(F)C3)cc2o1]

Synthesized and purified analogously to Example 150 using 2-ethoxy-5-ethylbenzene-1-sulfonamide (99.6 mg, 434 μmol) instead of 5-tert-butyl-2-methoxybenzene-1-sulfonamide to give 122 mg (65% yield) of the title compound as an offwhite solid. ¹H-NMR (400 MHz, DMSO-d₆) δ [ppm]: 1.168 (7.13), 1.187 (16.00), 1.204 (11.94), 1.221 (14.33), 1.239 (6.81), 2.074 (0.61), 2.083 (0.97), 2.610 (1.69), 2.629 (4.85), 2.647 (4.67), 2.667 (1.67), 3.944 (1.35), 3.964 (1.55), 3.969 (1.54), 4.000 (1.30), 4.004 (1.33), 4.025 (1.53), 4.029 (1.46), 4.094 (1.80), 4.111 (5.49), 4.129 (5.36), 4.146 (1.62), 4.186 (1.26), 4.199 (1.47), 4.211 (1.18), 4.226 (1.16), 4.239 (1.30), 4.251 (1.42), 4.264 (1.16), 4.277 (1.02), 5.415 (0.57), 5.422 (0.78), 5.430 (0.93), 5.437 (0.71), 5.444 (0.46), 5.559 (0.51), 5.566 (0.75), 5.574 (0.93), 5.580 (0.73), 5.587 (0.52), 6.428 (2.21), 6.432 (2.44), 6.457 (2.11), 6.461 (2.61), 6.481 (4.95), 7.121 (3.04), 7.143 (3.37), 7.467 (1.79), 7.473 (1.83), 7.489 (1.56), 7.494 (1.55), 7.711 (4.43), 7.717 (4.17), 8.059 (1.50), 12.432 (0.99), LC-MS (method 1): Rₜ=1.34 min; MS (ESIpos): m/z=465 [M+H]⁺

### Example 154

### N-(2-Chloroquinoline-8-sulfonyl)-4-fluoro-6-(3-fluoroazetidin-1-yl)-1-benzofuran-2-carboxamide

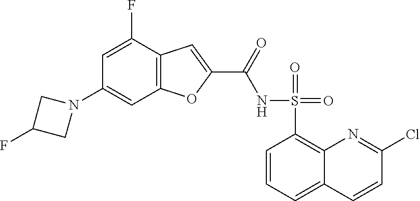 [SMILES: O=C(NS(=O)(=O)c1cccc2ccc(Cl)nc12)c1cc2c(F)cc(N3CC(F)C3)cc2o1]

Synthesized and purified analogously to Example 150 using 2-chloroquinoline-8-sulfonamide (105 mg, 434 μmol) instead of 5-tert-butyl-2-methoxybenzene-1-sulfonamide to give 77.0 mg (40% yield) of the title compound as an offwhite solid. ¹H-NMR (400 MHz, DMSO-d₆) δ [ppm]: 1.225 (0.60), 1.907 (0.46), 2.074 (8.68), 2.083 (9.28), 3.920 (3.60), 3.924 (4.06), 3.944 (5.02), 3.948 (5.23), 3.951 (4.99), 3.980 (4.03), 3.984 (4.18), 4.005 (4.81), 4.009 (4.85), 4.166 (3.77), 4.178 (4.55), 4.181 (4.62), 4.191 (3.88), 4.203 (3.68), 4.206 (3.84), 4.219 (4.29), 4.231 (4.37), 4.234 (4.42), 4.241 (3.78), 4.245 (3.92), 4.256 (3.36), 4.258 (3.39), 5.393 (0.80), 5.401 (1.68), 5.408 (2.39), 5.415 (3.01), 5.422 (2.48), 5.429 (1.77), 5.437 (0.97), 5.537 (0.73), 5.544 (1.49), 5.551 (2.32), 5.558 (3.01), 5.565 (2.58), 5.573 (1.96), 5.581 (1.05), 6.416 (5.80), 6.419 (8.40), 6.438 (16.00), 6.445 (11.60), 6.468 (0.92), 6.494 (0.43), 6.499 (0.45), 6.528 (0.54), 6.652 (0.54), 6.807 (0.51), 6.810 (0.52), 6.813 (0.53), 6.874 (0.44), 6.875 (0.44), 6.879 (0.48), 6.881 (0.46), 7.695 (0.78), 7.700 (0.83), 7.720 (15.44), 7.742 (15.95), 7.776 (0.82), 7.782 (0.80), 7.862 (6.91), 7.882 (11.15), 7.901 (7.56), 8.161 (3.81), 8.410 (6.98), 8.429 (6.33), 8.431 (6.46), 8.558 (8.22), 8.561 (8.62), 8.576 (8.17), 8.580 (8.09), 8.594 (15.04), 8.616 (14.47), 12.947 (0.76), LC-MS (method 1): Rₜ=1.24 min; MS (ESIpos): m/z=478 [M+H]⁺

### Example 155

### 4-Fluoro-6-(3-fluoroazetidin-1-yl)-N-{2-[(propan-2-yl)oxy]benzene-1-sulfonyl}-1-benzofuran-2-carboxamide

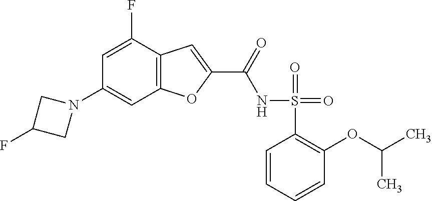 [SMILES: CC(C)Oc1ccccc1S(=O)(=O)NC(=O)c1cc2c(F)cc(N3CC(F)C3)cc2o1]

Synthesized and purified analogously to Example 150 using 2-[(propan-2-yl)oxy]benzene-1-sulfonamide (93.5 mg, 434 μmol) instead of 5-tert-butyl-2-methoxybenzene-1-sulfonamide to give 107 mg (57% yield) of the title compound as an offwhite solid. ¹H-NMR (400 MHz, DMSO-d₆) δ [ppm]: 1.173 (15.88), 1.189 (16.00), 2.083 (5.62), 3.945 (0.94), 3.965 (1.08), 3.969 (1.09), 4.001 (0.91), 4.005 (0.93), 4.026 (1.08), 4.030 (1.06), 4.186 (0.85), 4.201 (1.02), 4.210 (0.84), 4.226 (0.81), 4.239 (0.91), 4.252 (0.98), 4.264 (0.84), 4.277 (0.73), 4.789 (0.95), 4.804 (1.28), 4.819 (0.95), 5.422 (0.54), 5.428 (0.64), 5.436 (0.51), 5.565 (0.52), 5.572 (0.64), 5.579 (0.53), 6.430 (1.43), 6.433 (1.58), 6.459 (1.39), 6.463 (1.68), 6.487 (3.30), 7.082 (1.04), 7.101 (2.05), 7.120 (1.15), 7.225 (1.71), 7.246 (1.88), 7.602 (0.76), 7.605 (0.81), 7.624 (1.37), 7.641 (0.69), 7.645 (0.67), 7.889 (1.73), 7.892 (1.79), 7.908 (1.72), 7.912 (1.64), 8.081 (1.35), 12.385 (0.89), LC-MS (method 1): Rₜ=1.26 min; MS (ESIpos): m/z=451 [M+H]⁺

### Example 156

### 6-(Azetidin-1-yl)-4-fluoro-N-(2-methoxy-4-methylbenzene-1-sulfonyl)-1-benzofuran-2-carboxamide

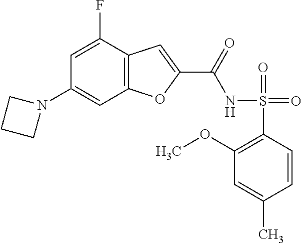 [SMILES: COc1cc(C)ccc1S(=O)(=O)NC(=O)c1cc2c(F)cc(N3CCC3)cc2o1]

Synthesized and purified analogously to Example 127 using 2-methoxy-4-methylbenzene-1-sulfonamide (128 mg, 638 μmol) instead of 5-tert-butyl-2-methoxybenzene-1-sulfonamide to give 28.0 mg (15% yield) of the title compound as an offwhite solid. ¹H-NMR (400 MHz, DMSO-d₆) δ [ppm]: 1.231 (0.64), 2.084 (11.19), 2.286 (0.49), 2.304 (1.53), 2.323 (2.58), 2.341 (1.75), 2.372 (13.05), 2.523 (1.34), 2.669 (0.43), 3.565 (0.71), 3.815 (0.63), 3.821 (0.53), 3.840 (16.00), 3.868 (4.38), 3.887 (7.25), 3.905 (4.09), 6.308 (1.54), 6.312 (1.73), 6.341 (2.07), 6.352 (3.64), 6.940 (1.54), 6.960 (1.62), 7.050 (2.90), 7.758 (3.20), 7.778 (2.92), 8.002 (0.82), 12.436 (0.67), LC-MS (method 1): Rₜ=1.27 min; MS (ESIpos): m/z=419 [M+H]⁺

### Example 157

### 4-Fluoro-6-(3-fluoroazetidin-1-yl)-N-{5-methyl-2-[(propan-2-yl)oxy]benzene-1-sulfonyl}-1-benzofuran-2-carboxamide

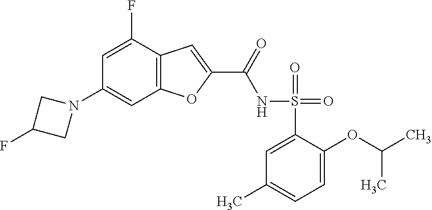 [SMILES: Cc1ccc(OC(C)C)c(S(=O)(=O)NC(=O)c2cc3c(F)cc(N4CC(F)C4)cc3o2)c1]

Synthesized and purified analogously to Example 150 using commercially available 5-methyl-2-[(propan-2-yl)oxy]benzene-1-sulfonamide (CAS: 1094691-36-7, 99.6 mg, 434 μmol) instead of 5-tert-butyl-2-methoxybenzene-1-sulfonamide to give 105 mg (55% yield) of the title compound as a yellow solid. ¹H-NMR (400 MHz, DMSO-d₆) δ [ppm]: 1.145 (16.00), 1.161 (15.89), 2.083 (14.94), 2.318 (10.64), 2.518 (1.16), 2.522 (0.74), 3.944 (0.76), 3.965 (0.88), 3.969 (0.87), 4.000 (0.73), 4.005 (0.76), 4.026 (0.87), 4.029 (0.84), 4.187 (0.70), 4.199 (0.84), 4.201 (0.83), 4.211 (0.67), 4.226 (0.65), 4.237 (0.73), 4.252 (0.79), 4.264 (0.67), 4.277 (0.58), 4.730 (0.65), 4.745 (0.87), 4.760 (0.64), 5.421 (0.47), 5.429 (0.56), 5.436 (0.42), 5.565 (0.45), 5.572 (0.55), 5.579 (0.44), 6.427 (1.17), 6.431 (1.28), 6.457 (1.11), 6.460 (1.33), 6.486 (2.84), 7.116 (1.16), 7.138 (1.32), 7.413 (0.84), 7.431 (0.73), 7.692 (2.09), 7.696 (2.03), 8.075 (0.62), 12.338 (0.77), LC-MS (method 1): Rₜ=1.33 min; MS (ESIpos): m/z=465 [M+H]⁺

### Example 158

### 4-Fluoro-6-(3-fluoroazetidin-1-yl)-N-(2-methoxy-4-methylbenzene-1-sulfonyl)-1-benzofuran-2-carboxamide

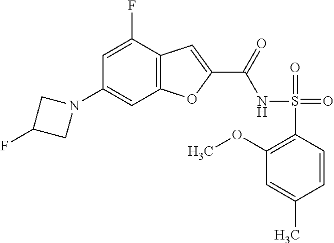 [SMILES: COc1cc(C)ccc1S(=O)(=O)NC(=O)c1cc2c(F)cc(N3CC(F)C3)cc2o1]

Synthesized and purified analogously to Example 150 using 2-methoxy-4-methylbenzene-1-sulfonamide (87.4 mg, 434 μmol) instead of 5-tert-butyl-2-methoxybenzene-1-sulfonamide to give 112 mg (62% yield) of the title compound as an offwhite solid. ¹H-NMR (400 MHz, DMSO-d₆) δ [ppm]: 2.083 (6.93), 2.328 (0.56), 2.371 (11.62), 2.522 (0.69), 3.815 (0.60), 3.821 (0.47), 3.840 (16.00), 3.935 (0.77), 3.939 (0.82), 3.960 (0.97), 3.964 (0.98), 3.967 (0.94), 3.996 (0.81), 4.000 (0.85), 4.021 (1.02), 4.025 (0.94), 4.183 (0.77), 4.195 (0.91), 4.197 (0.92), 4.207 (0.75), 4.222 (0.73), 4.235 (0.81), 4.247 (0.88), 4.258 (0.73), 4.261 (0.76), 4.273 (0.65), 5.421 (0.50), 5.428 (0.60), 5.436 (0.46), 5.565 (0.47), 5.572 (0.59), 5.579 (0.48), 6.420 (1.44), 6.424 (1.59), 6.449 (1.39), 6.453 (1.64), 6.486 (2.99), 6.942 (1.43), 6.962 (1.47), 7.050 (2.71), 7.760 (3.24), 7.780 (2.94), 8.012 (1.35), LC-MS (method 1): Rₜ=1.22 min; MS (ESIpos): m/z=437 [M+H]⁺

### Example 159

### 4-Fluoro-6-(3-fluoroazetidin-1-yl)-N-(2,2,3,3-tetrafluoro-2,3-dihydro-1,4-benzodioxine-5-sulfonyl)-1-benzofuran-2-carboxamide

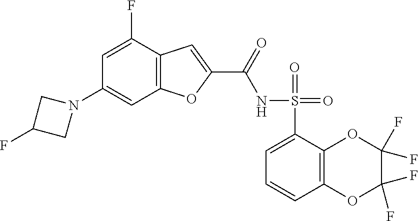 [SMILES: O=C(NS(=O)(=O)c1cccc2c1OC(F)(F)C(F)(F)O2)c1cc2c(F)cc(N3CC(F)C3)cc2o1]

Synthesized and purified analogously to Example 150 using 2,2,3,3-tetrafluoro-2,3-dihydro-1,4-benzodioxine-5-sulfonamide (125 mg, 434 μmol) instead of 5-tert-butyl-2-methoxybenzene-1-sulfonamide to give 128 mg (60% yield) of the title compound as an offwhite solid. ¹H-NMR (400 MHz, DMSO-d₆) δ [ppm]: 1.907 (0.40), 2.073 (7.79), 2.083 (16.00), 2.518 (3.31), 2.522 (2.10), 3.386 (0.74), 3.433 (0.77), 3.585 (0.84), 3.874 (0.74), 3.889 (0.77), 3.947 (3.24), 3.967 (3.48), 3.972 (3.52), 3.976 (3.34), 4.000 (2.81), 4.003 (2.88), 4.008 (3.08), 4.029 (3.41), 4.033 (3.23), 4.191 (2.69), 4.202 (3.28), 4.206 (3.21), 4.213 (2.52), 4.216 (2.62), 4.227 (2.45), 4.230 (2.51), 4.244 (2.80), 4.255 (2.99), 4.258 (2.97), 4.266 (2.46), 4.269 (2.58), 4.283 (2.24), 5.409 (0.63), 5.417 (1.21), 5.424 (1.64), 5.432 (2.03), 5.439 (1.52), 5.446 (1.04), 5.453 (0.52), 5.553 (0.54), 5.561 (1.03), 5.568 (1.60), 5.575 (2.01), 5.582 (1.60), 5.589 (1.20), 5.597 (0.55), 6.428 (4.80), 6.433 (5.52), 6.457 (4.50), 6.462 (5.96), 6.482 (8.73), 6.485 (10.23), 6.528 (0.46), 6.651 (0.41), 6.810 (0.41), 6.873 (0.42), 6.879 (0.41), 7.568 (5.13), 7.589 (9.81), 7.609 (6.36), 7.694 (0.59), 7.700 (0.61), 7.776 (0.61), 7.782 (0.63), 7.826 (5.52), 7.830 (5.96), 7.847 (4.94), 7.851 (4.83), 7.918 (5.38), 7.941 (7.04), 7.944 (6.96), 7.961 (6.31), 7.965 (5.61), LC-MS (method 2): Rₜ=0.87 min; MS (ESIpos): m/z=523 [M+H]⁺

### Example 160

### 6-[2-(Difluoromethyl)azetidin-1-yl]-4-fluoro-N-(2-methylquinoline-8-sulfonyl)-1-benzofuran-2-carboxamide (rac)

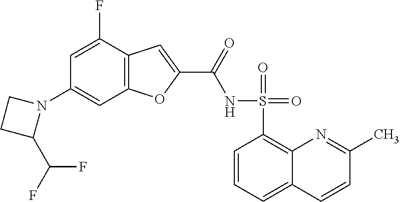 [SMILES: Cc1ccc2cccc(S(=O)(=O)NC(=O)c3cc4c(F)cc(N5CCC5C(F)F)cc4o3)c2n1]

Synthesized and purified analogously to Example 127 using 6-[2-(difluoromethyl)azetidin-1-yl]-4-fluoro-1-benzofuran-2-carboxylic acid (rac) (Intermediate 21, 100 mg, 351 μmol) and 2-methylquinoline-8-sulfonamide (85.7 mg, 386 μmol) instead of 6-(azetidin-1-yl)-4-fluoro-1-benzofuran-2-carboxylic acid and 5-tert-butyl-2-methoxybenzene-1-sulfonamide to give 52.7 mg (30% yield) of the title compound as a yellow solid. ¹H-NMR (400 MHz, DMSO-d₆) δ [ppm]: 2.322 (0.43), 2.326 (0.47), 2.332 (0.52), 2.342 (0.41), 2.349 (0.61), 2.370 (0.56), 2.403 (0.54), 2.415 (0.59), 2.425 (0.44), 2.437 (0.43), 2.518 (0.77), 2.523 (0.48), 2.679 (16.00), 3.691 (0.40), 3.710 (0.88), 3.732 (0.85), 3.751 (0.40), 3.977 (0.45), 3.989 (0.59), 3.998 (0.77), 4.009 (0.83), 4.018 (0.48), 4.464 (0.46), 6.204 (0.51), 6.212 (0.48), 6.343 (0.89), 6.352 (0.91), 6.471 (1.44), 6.476 (1.62), 6.490 (0.55), 6.501 (1.33), 6.506 (1.56), 6.532 (2.41), 6.533 (2.77), 7.513 (3.35), 7.534 (3.30), 7.732 (1.69), 7.751 (2.41), 7.770 (1.73), 8.160 (2.20), 8.286 (1.64), 8.290 (1.74), 8.307 (1.61), 8.311 (1.50), 8.387 (3.09), 8.407 (2.89), 8.452 (2.09), 8.456 (2.20), 8.471 (2.12), 8.475 (1.81); LC-MS (method 1): Rₜ=1.20 min; MS (ESIpos): m/z=490 [M+H]⁺

### Example 161

### 4-Fluoro-6-[2-(1-hydroxy-1-methyl-ethyl)azetidin-1-yl]-N-[(2-methyl-8-quinolyl)sulfonyl]benzofuran-2-carboxamide (rac)

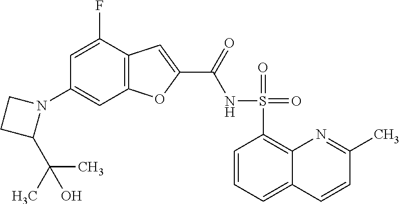 [SMILES: Cc1ccc2cccc(S(=O)(=O)NC(=O)c3cc4c(F)cc(N5CCC5C(C)(C)O)cc4o3)c2n1]

Synthesized and purified analogously to Example 127 using 4-fluoro-6-[2-(2-hydroxypropan-2-yl)azetidin-1-yl]-1-benzofuran-2-carboxylic acid (rac) (Intermediate 24, 100 mg, 341 μmol) and commercially available 2-methylquinoline-8-sulfonamide (CAS: 157686-27-6, 83.4 mg, 375 μmol) instead of 6-(azetidin-1-yl)-4-fluoro-1-benzofuran-2-carboxylic acid and 5-tert-butyl-2-methoxybenzene-1-sulfonamide to give 85.0 mg (48% yield) of the title compound as a yellow solid. ¹H-NMR (400 MHz, DMSO-d₆) δ [ppm]: 1.066 (10.44), 1.164 (12.36), 1.225 (0.41), 2.029 (0.41), 2.035 (0.40), 2.040 (0.40), 2.052 (0.62), 2.057 (0.68), 2.074 (12.66), 2.083 (2.12), 2.254 (0.54), 2.266 (0.57), 2.282 (0.50), 2.289 (0.48), 2.322 (0.40), 2.326 (0.51), 2.518 (1.73), 2.522 (1.14), 2.665 (0.54), 2.669 (0.86), 2.681 (16.00), 2.696 (0.57), 3.345 (0.55), 3.519 (0.53), 3.538 (0.98), 3.559 (0.93), 3.579 (0.48), 3.843 (0.47), 3.856 (0.60), 3.865 (0.88), 3.876 (0.91), 3.885 (0.52), 3.897 (0.41), 3.927 (0.98), 3.943 (1.18), 3.949 (1.22), 3.965 (0.94), 4.839 (2.18), 6.692 (2.81), 6.767 (1.28), 6.799 (1.26), 7.511 (3.18), 7.531 (3.29), 7.727 (1.49), 7.747 (2.53), 7.766 (1.62), 8.125 (1.31), 8.282 (1.56), 8.285 (1.62), 8.302 (1.48), 8.305 (1.42), 8.382 (3.06), 8.404 (2.88), 8.444 (2.03), 8.448 (2.00), 8.463 (1.93), 8.466 (1.79); LC-MS (method 1): Rₜ=1.16 min; MS (ESIpos): m/z=498 [M+H]⁺

### Example 162

### N-(2-ethoxybenzene-1-sulfonyl)-4-fluoro-6-[2-(2-hydroxypropan-2-yl)azetidin-1-yl]-1-benzofuran-2-carboxamide (rac)

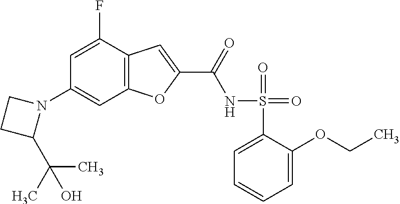 [SMILES: CCOc1ccccc1S(=O)(=O)NC(=O)c1cc2c(F)cc(N3CCC3C(C)(C)O)cc2o1]

Synthesized and purified analogously to Example 161 using 2-ethoxybenzene-1-sulfonamide (75.5 mg, 375 μmol) instead of 2-methylquinoline-8-sulfonamide to give 113 mg (63% yield) of the title compound as a yellow solid. ¹H-NMR (400 MHz, DMSO-d₆) δ [ppm]: 1.080 (14.38), 1.137 (0.63), 1.182 (16.00), 1.192 (3.71), 1.221 (6.13), 1.239 (13.58), 1.256 (6.24), 2.048 (0.61), 2.054 (0.58), 2.065 (0.93), 2.074 (7.43), 2.083 (9.73), 2.092 (0.81), 2.258 (0.46), 2.270 (0.71), 2.281 (0.75), 2.292 (0.63), 2.297 (0.64), 2.304 (0.61), 2.308 (0.59), 2.322 (0.50), 2.327 (0.53), 2.331 (0.52), 2.518 (1.45), 2.522 (0.90), 3.545 (0.50), 3.564 (1.10), 3.586 (1.06), 3.605 (0.50), 3.863 (0.78), 3.875 (1.06), 3.884 (1.28), 3.896 (1.32), 3.905 (0.67), 3.917 (0.51), 3.949 (1.24), 3.966 (1.49), 3.971 (1.53), 3.987 (1.15), 4.132 (1.47), 4.149 (4.48), 4.167 (4.47), 4.184 (1.37), 4.860 (2.54), 6.756 (3.64), 6.777 (1.72), 6.809 (2.22), 6.830 (0.64), 6.832 (0.54), 6.836 (0.63), 6.837 (0.52), 7.108 (1.23), 7.127 (2.44), 7.146 (1.33), 7.200 (2.09), 7.221 (2.27), 7.615 (0.87), 7.619 (0.92), 7.637 (1.53), 7.655 (0.76), 7.726 (0.89), 7.731 (0.87), 7.888 (2.37), 7.892 (2.42), 7.908 (2.26), 7.912 (2.16), 8.025 (0.99), 12.428 (0.90), LC-MS (method 1): Rₜ=1.23 min; MS (ESIpos): m/z=477 [M+H]⁺

### Example 163

### 6-[2-(Difluoromethyl)azetidin-1-yl]-N-(2-ethoxybenzene-1-sulfonyl)-4-fluoro-1-benzofuran-2-carboxamide (rac)

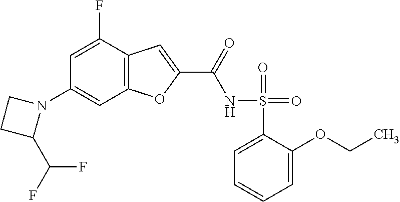 [SMILES: CCOc1ccccc1S(=O)(=O)NC(=O)c1cc2c(F)cc(N3CCC3C(F)F)cc2o1]

Synthesized and purified analogously to Example 160 using 2-ethoxybenzene-1-sulfonamide (77.6 mg, 386 μmol) instead of 2-methylquinoline-8-sulfonamide to give 37.3 mg (22% yield) of the title compound as a white solid. ¹H-NMR (400 MHz, DMSO-d₆) δ [ppm]: 1.221 (6.65), 1.239 (16.00), 1.256 (6.86), 2.322 (0.41), 2.327 (0.43), 2.337 (0.50), 2.349 (0.54), 2.366 (0.87), 2.387 (0.81), 2.396 (0.48), 2.404 (0.58), 2.419 (0.78), 2.431 (0.83), 2.441 (0.65), 2.447 (0.60), 2.454 (0.66), 2.518 (1.24), 2.523 (0.79), 3.718 (0.52), 3.737 (1.21), 3.760 (1.19), 3.779 (0.54), 4.000 (0.63), 4.012 (0.81), 4.021 (1.08), 4.031 (1.19), 4.040 (0.69), 4.053 (0.54), 4.136 (1.68), 4.154 (5.43), 4.171 (5.44), 4.189 (1.62), 4.468 (0.47), 4.490 (0.66), 4.515 (0.47), 6.223 (0.74), 6.231 (0.72), 6.362 (1.29), 6.370 (1.30), 6.488 (2.04), 6.492 (2.18), 6.500 (0.74), 6.509 (0.80), 6.518 (2.00), 6.522 (2.09), 6.589 (3.95), 7.112 (1.37), 7.114 (1.49), 7.133 (2.69), 7.150 (1.56), 7.152 (1.53), 7.206 (2.37), 7.226 (2.65), 7.623 (1.20), 7.627 (1.26), 7.641 (1.46), 7.645 (1.72), 7.662 (1.02), 7.667 (1.00), 7.894 (2.88), 7.899 (2.95), 7.915 (2.69), 7.919 (2.54), 8.071 (2.08), 12.496 (0.75); LC-MS (method 1): Rₜ=1.27 min; MS (ESIpos): m/z=469 [M+H]⁺

### Example 164

### N-{2,6-difluoro-4-[methyl(propan-2-yl)amino]benzene-1-sulfonyl}-4-fluoro-6-(3-fluoroazetidin-1-yl)-1-benzofuran-2-carboxamide

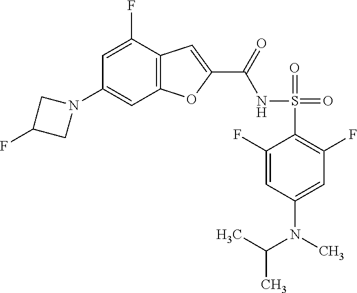 [SMILES: CC(C)N(C)c1cc(F)c(S(=O)(=O)NC(=O)c2cc3c(F)cc(N4CC(F)C4)cc3o2)c(F)c1]

Synthesized and purified analogously to Example 150 using 2,6-difluoro-4-[methyl(propan-2-yl)amino]benzene-1-sulfonamide (72.0 mg, 272 μmol) instead of 5-tert-butyl-2-methoxybenzene-1-sulfonamide to give 60.0 mg (52% yield) as an offwhite solid. ¹H-NMR (400 MHz, DMSO-d₆) δ [ppm]: 1.105 (8.49), 1.121 (8.71), 1.137 (0.72), 2.084 (16.00), 2.518 (1.31), 2.522 (0.79), 2.763 (9.36), 3.948 (0.45), 3.952 (0.47), 3.972 (0.58), 3.977 (0.57), 3.980 (0.55), 4.008 (0.47), 4.013 (0.49), 4.033 (0.56), 4.038 (0.53), 4.156 (0.51), 4.173 (0.69), 4.189 (0.67), 4.206 (0.70), 4.220 (0.47), 4.235 (0.42), 4.248 (0.47), 4.259 (0.52), 4.270 (0.43), 4.273 (0.44), 6.429 (0.87), 6.433 (0.95), 6.458 (0.84), 6.462 (0.99), 6.490 (1.63), 6.492 (1.91), 6.512 (1.38), 6.547 (1.37), 7.962 (0.57); LC-MS (method 1): Rₜ=1.30 min; MS (ESIpos): m/z=500 [M+H]⁺

### Example 165

### N-{2,4-difluoro-6-[methyl(propan-2-yl)amino]benzene-1-sulfonyl}-4-fluoro-6-(3-fluoroazetidin-1-yl)-1-benzofuran-2-carboxamide

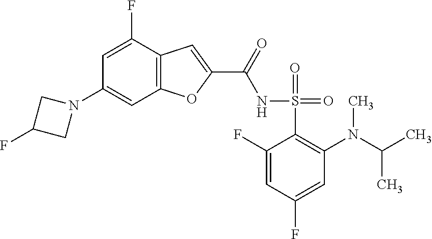 [SMILES: CC(C)N(C)c1cc(F)cc(F)c1S(=O)(=O)NC(=O)c1cc2c(F)cc(N3CC(F)C3)cc2o1]

Synthesized and purified analogously to Example 150 using 2,4-difluoro-6-[methyl(propan-2-yl)amino]benzene-1-sulfonamide (76.0 mg, 288 μmol) instead of 5-tert-butyl-2-methoxybenzene-1-sulfonamide to give 65.0 mg (95% purity, 52% yield) of the title compound as an offwhite solid. ¹H-NMR (400 MHz, DMSO-d₆) δ [ppm]: 1.042 (0.41), 1.174 (15.76), 1.190 (16.00), 1.230 (1.11), 1.907 (0.58), 2.074 (2.43), 2.083 (9.99), 2.518 (7.94), 2.523 (5.27), 3.050 (6.11), 3.165 (0.44), 3.504 (0.87), 3.841 (0.61), 3.856 (1.57), 3.872 (2.17), 3.889 (1.58), 3.905 (0.61), 3.930 (1.74), 3.951 (2.08), 3.956 (2.05), 3.987 (1.71), 3.992 (1.76), 4.011 (2.02), 4.016 (1.96), 4.177 (1.57), 4.189 (1.95), 4.201 (1.61), 4.213 (1.48), 4.227 (1.71), 4.244 (1.89), 4.254 (1.64), 4.268 (1.41), 5.416 (0.73), 5.423 (1.01), 5.430 (1.26), 5.438 (0.97), 5.445 (0.69), 5.567 (0.97), 5.574 (1.22), 5.581 (1.00), 5.589 (0.74), 6.389 (3.00), 6.393 (3.28), 6.418 (2.98), 6.423 (3.29), 6.478 (5.40), 6.480 (6.52), 7.316 (0.88), 7.466 (0.77), 7.602 (1.82), LC-MS (method 1): Rₜ=1.08 min; MS (ESIneg): m/z=498 [M−H]⁻

### Example 166

### N-{2,3-difluoro-4-[methyl(propan-2-yl)amino]benzene-1-sulfonyl}-4-fluoro-6-(3-fluoroazetidin-1-yl)-1-benzofuran-2-carboxamide

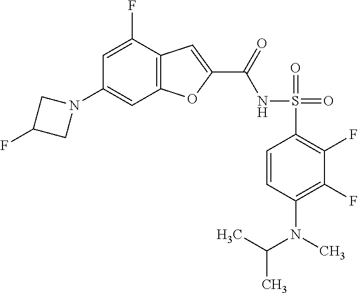 [SMILES: CC(C)N(C)c1ccc(S(=O)(=O)NC(=O)c2cc3c(F)cc(N4CC(F)C4)cc3o2)c(F)c1F]

Synthesized and purified analogously to Example 150 using 2,3-difluoro-4-[methyl(propan-2-yl)amino]benzene-1-sulfonamide (Intermediate 43, 86.0 mg, 325 μmol) instead of 5-tert-butyl-2-methoxybenzene-1-sulfonamide to give 79.0 mg (98% purity, 57% yield) of the title compound as an offwhite solid. ¹H-NMR (400 MHz, DMSO-d₆) δ [ppm]: 1.137 (0.94), 1.149 (11.27), 1.166 (11.26), 2.083 (16.00), 2.518 (1.61), 2.522 (0.95), 2.793 (5.78), 2.798 (5.79), 3.945 (0.64), 3.950 (0.68), 3.974 (1.52), 3.990 (1.09), 4.006 (1.37), 4.031 (0.80), 4.035 (0.76), 4.192 (0.60), 4.204 (0.74), 4.207 (0.72), 4.217 (0.59), 4.232 (0.57), 4.245 (0.64), 4.257 (0.70), 4.267 (0.58), 4.270 (0.60), 4.282 (0.52), 5.433 (0.46), 5.576 (0.47), 6.425 (1.13), 6.429 (1.26), 6.454 (1.08), 6.458 (1.31), 6.486 (2.47), 6.829 (0.52), 6.850 (1.00), 6.870 (0.54), 7.528 (0.52), 7.552 (0.96), 7.571 (0.52), 7.936 (0.89); LC-MS (method 1): Rₜ=1.39 min; MS (ESIpos): m/z=500 [M+H]⁺

### Example 167

### 6-(Azetidin-1-yl)-N-[2-(ethanesulfonyl)imidazo[1,2-a]pyridine-3-sulfonyl]-4-fluoro-1-benzofuran-2-carboxamide

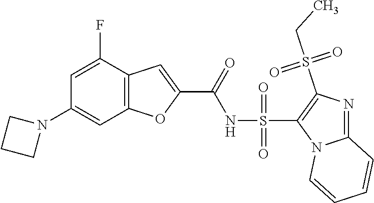 [SMILES: CCS(=O)(=O)c1nc2ccccn2c1S(=O)(=O)NC(=O)c1cc2c(F)cc(N3CCC3)cc2o1]

Synthesized and purified analogously to Example 127 using 2-(ethanesulfonyl)imidazo[1,2-a]pyridine-3-sulfonamide (157 mg, 542 μmol) instead of 5-tert-butyl-2-methoxybenzene-1-sulfonamide to give 31.5 mg (90% purity, 15% yield) of the title compound as an offwhite solid. ¹H-NMR (400 MHz, DMSO-d₆) δ [ppm]: 1.122 (4.60), 1.140 (10.37), 1.158 (4.68), 1.229 (0.72), 2.074 (14.48), 2.083 (8.38), 2.272 (0.53), 2.294 (1.90), 2.312 (4.41), 2.331 (5.55), 2.349 (3.34), 2.368 (1.07), 2.669 (0.89), 3.671 (2.36), 3.689 (5.29), 3.707 (5.29), 3.726 (2.55), 3.847 (5.93), 3.866 (10.18), 3.872 (12.02), 3.890 (16.00), 3.908 (9.48), 4.118 (0.68), 4.137 (0.69), 6.263 (1.63), 6.266 (1.89), 6.299 (7.40), 6.328 (2.91), 6.332 (3.03), 6.415 (5.60), 7.354 (1.00), 7.369 (1.96), 7.389 (1.16), 7.533 (8.11), 7.535 (8.10), 7.553 (0.45), 7.574 (1.71), 7.658 (1.09), 7.680 (1.52), 7.698 (1.16), 7.887 (2.39), 7.909 (1.98), 9.104 (2.62), 9.122 (2.49); LC-MS (method 1): Rₜ=1.03 min; MS (ESIneg): m/z=505 [M−H]⁻

### Example 168

### 6-(Azetidin-1-yl)-N-(6,8-dichloroimidazo[1,2-a]pyridine-3-sulfonyl)-4-fluoro-1-benzofuran-2-carboxamide

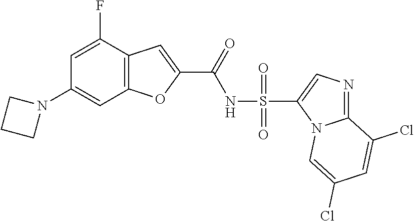 [SMILES: O=C(NS(=O)(=O)c1cnc2c(Cl)cc(Cl)cn12)c1cc2c(F)cc(N3CCC3)cc2o1]

Synthesized and purified analogously to Example 127 using 6,8-dichloroimidazo[1,2-a]pyridine-3-sulfonamide (144 mg, 542 μmol) instead of 5-tert-butyl-2-methoxybenzene-1-sulfonamide to give 12.3 mg (6% yield) of the title compound as an offwhite solid. ¹H-NMR (400 MHz, DMSO-d₆) δ [ppm]: 0.850 (0.41), 1.230 (3.05), 2.074 (1.87), 2.295 (3.64), 2.312 (5.36), 2.331 (5.13), 2.669 (1.40), 3.849 (9.58), 3.867 (16.00), 3.885 (9.60), 4.109 (0.72), 6.256 (3.47), 6.285 (3.73), 6.320 (7.49), 7.436 (3.84), 7.976 (6.12), 8.133 (1.38), 8.153 (6.13), 8.990 (6.97), 8.993 (7.15); LC-MS (method 1): Rₜ=1.19 min; MS (ESIpos): m/z=483 [M+H]⁺

### Example 169

### 1-{2-[(2-Ethoxybenzene-1-sulfonyl)carbamoyl]-4-fluoro-1-benzofuran-6-yl}azetidine-2-carboxamide (rac)

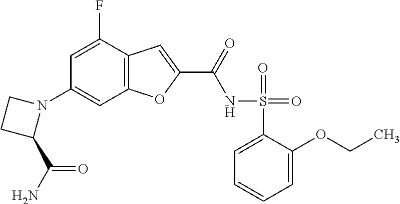 [SMILES: CCOc1ccccc1S(=O)(=O)NC(=O)c1cc2c(F)cc(N3CC[C@@H]3C(N)=O)cc2o1]

Synthesized analogously to Example 127 using 6-(2-carbamoylazetidin-1-yl)-4-fluoro-1-benzofuran-2-carboxylic acid (Intermediate 46, 8.90 mg, 32.0 μmol) and 2-ethoxybenzene-1-sulfonamide (7.08 mg, 35.2 μmol) instead of 6-(Azetidin-1-yl)-4-fluoro-1-benzofuran-2-carboxylic acid and commercially available 5-tert-butyl-2-methoxybenzene-1-sulfonamide to give 2.60 mg (16% yield) of the title compound as a white solid. ¹H-NMR (400 MHz, DMSO-d₆) δ [ppm]: 0.834 (0.71), 1.207 (2.59), 1.224 (5.48), 1.241 (3.75), 1.272 (1.22), 1.293 (1.09), 1.311 (0.94), 1.359 (1.60), 1.376 (2.61), 1.394 (1.51), 1.420 (0.54), 2.304 (0.57), 2.327 (1.23), 2.540 (16.00), 2.669 (0.90), 3.020 (0.96), 3.732 (0.68), 3.751 (0.71), 3.955 (0.61), 3.965 (0.62), 4.118 (1.32), 4.135 (1.37), 4.151 (0.66), 4.180 (0.42), 4.197 (1.06), 4.214 (1.02), 4.374 (0.48), 4.394 (0.72), 4.412 (0.48), 6.265 (0.76), 6.294 (0.77), 6.369 (1.95), 6.913 (1.29), 7.036 (0.61), 7.055 (0.41), 7.091 (0.83), 7.109 (0.48), 7.157 (0.60), 7.178 (0.68), 7.190 (0.77), 7.212 (0.72), 7.278 (1.24), 7.543 (0.61), 7.565 (0.61), 7.600 (1.50), 7.718 (0.49), 7.737 (0.50), 7.866 (0.93), 7.884 (0.88); LC-MS (method 1): Rₜ=0.99 min; MS (ESIpos): m/z=462 [M+H]⁺

### Example 170

### 6-(azetidin-1-yl)-N-{5-bromo-2-[(propan-2-yl)oxy]benzene-1-sulfonyl}-4-fluoro-1-benzofuran-2-carboxamide

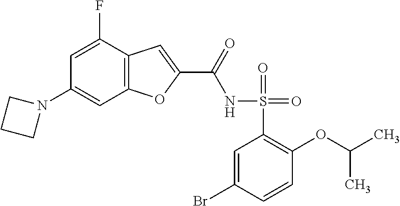 [SMILES: CC(C)Oc1ccc(Br)cc1S(=O)(=O)NC(=O)c1cc2c(F)cc(N3CCC3)cc2o1]

Synthesized analogously to Example 127 using 6-(azetidin-1-yl)-4-fluoro-1-benzofuran-2-carboxylic acid (500 mg, 2.13 mmol) and 5-bromo-2-[(propan-2-yl)oxy]benzene-1-sulfonamide (938 mg, 3.19 mmol) instead of 6-(Azetidin-1-yl)-4-fluoro-1-benzofuran-2-carboxylic acid and 5-tert-butyl-2-methoxybenzene-1-sulfonamide. The reaction mixture was concentrated, diluted with water and extracted with dichloromethane. The organic layer was washed with aq. HCl (1 M) dried over sodium sulfate and concentrated in vacuo. The residue was stirred in hexane and the undissolved solid was collected by filtration to give 882 mg (77% yield) of the title compound as a yellow solid. ¹H-NMR (400 MHz, DMSO-d₆) δ [ppm]: 1.161 (16.00), 1.176 (15.73), 1.312 (0.43), 1.328 (0.45), 2.307 (1.13), 2.326 (2.45), 2.336 (0.74), 2.344 (1.26), 2.518 (3.40), 2.523 (2.16), 2.664 (0.64), 2.669 (0.91), 2.673 (0.67), 3.875 (3.42), 3.894 (5.71), 3.912 (3.28), 4.785 (0.54), 4.799 (0.72), 4.815 (0.54), 6.317 (1.05), 6.320 (1.23), 6.351 (4.00), 7.230 (0.91), 7.252 (0.99), 7.785 (0.66), 7.807 (0.61), 7.929 (3.52), 7.936 (3.23); LC-MS (method 1): Rₜ=1.42 min; MS (ESIneg): m/z=513 [M+H]⁻

### Example 171

### 6-(Azetidin-1-yl)-N-(2-ethylquinoline-8-sulfonyl)-4-fluoro-1-benzofuran-2-carboxamide

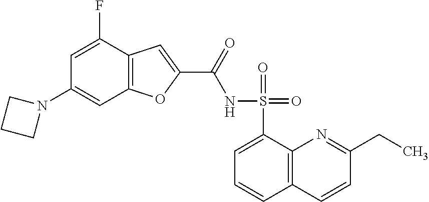 [SMILES: CCc1ccc2cccc(S(=O)(=O)NC(=O)c3cc4c(F)cc(N5CCC5)cc4o3)c2n1]

To a stirring solution of 6-(azetidin-1-yl)-4-fluoro-1-benzofuran-2-carboxamide (Intermediate 14, 50.0 mg, 213 μmol) in THF (2.0 ml) was added sodium hydride (20.5 mg, 50% purity, 426 μmol; CAS-RN:[7646-69-7]) at 0° C. The reaction mixture was then stirred at room temperature. After 30 min 2-ethylquinoline-8-sulfonyl chloride (65.5 mg, 256 μmol) was added and the resulting mixture was stirred for 3 h. After completion the reaction mixture was diluted with 2 mL acetonitril/water 7/3 and purified by preparative HPLC (water+0.1% HCOOH/acetonitrile) to give 10.3 mg (10% yield) of the title compound as an white solid. ¹H-NMR (400 MHz, DMSO-d₆) δ [ppm]: 1.128 (7.10), 1.137 (1.16), 1.147 (16.00), 1.166 (7.18), 1.231 (0.67), 1.810 (0.44), 1.955 (1.34), 2.081 (3.05), 2.084 (6.10), 2.270 (0.80), 2.288 (2.51), 2.306 (3.56), 2.326 (4.01), 2.332 (1.85), 2.343 (0.88), 2.518 (6.88), 2.522 (4.23), 2.664 (1.23), 2.669 (1.72), 2.673 (1.27), 2.781 (1.29), 2.930 (1.08), 2.941 (3.18), 2.948 (2.60), 2.967 (2.53), 2.984 (1.03), 3.128 (0.58), 3.845 (6.54), 3.863 (10.97), 3.880 (6.17), 5.758 (2.28), 6.290 (7.81), 6.314 (1.49), 7.495 (0.68), 7.547 (1.10), 7.566 (1.11), 7.739 (0.98), 8.100 (0.44), 8.286 (0.73), 8.402 (1.17), 8.422 (1.28), 8.441 (1.22); LC-MS (method 1): Rₜ=1.28 min; MS (ESIpos): m/z=454 [M+H]⁺

### Example 172

### 6-(Azetidin-1-yl)-N-(2,3-dihydro-1,4-benzodioxine-5-sulfonyl)-4-fluoro-1-benzofuran-2-carboxamide

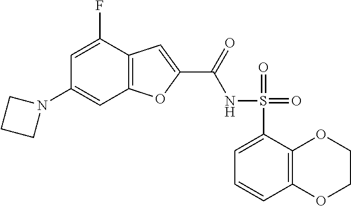 [SMILES: O=C(NS(=O)(=O)c1cccc2c1OCCO2)c1cc2c(F)cc(N3CCC3)cc2o1]

To a stirring solution of 6-(azetidin-1-yl)-4-fluoro-1-benzofuran-2-carboxamide (Intermediate 14, 80.0 mg, 342 μmol) in THF (3.2 ml) was added (32.8 mg, 50% purity, 683 μmol; CAS-RN:[7646-69-7]) at 0° C. The suspension was allowed to warm to room temperature and stirred for 30 min. 2,3-dihydro-1,4-benzodioxine-5-sulfonyl chloride (96.2 mg, 410 μmol) was added and the reaction mixture was stirred for 3 h at room temperature. After completion the mixture was evaporated and purified by HT-HPLC (acid) to give 9.00 mg (6% yield) of the title compound as a white solid. ¹H-NMR (400 MHz, DMSO-d₆) δ [ppm]: 2.074 (2.89), 2.154 (0.43), 2.291 (1.00), 2.308 (3.20), 2.327 (5.56), 2.345 (3.59), 2.363 (1.27), 2.523 (3.48), 2.669 (0.85), 3.874 (9.45), 3.892 (16.00), 3.911 (9.27), 4.268 (5.39), 4.276 (5.75), 4.280 (6.21), 4.310 (6.27), 4.315 (5.70), 4.322 (5.43), 6.316 (3.59), 6.320 (4.09), 6.345 (3.23), 6.349 (4.51), 6.364 (7.89), 6.997 (4.00), 7.017 (8.43), 7.037 (5.10), 7.181 (4.99), 7.184 (5.35), 7.201 (4.25), 7.205 (4.06), 7.441 (5.61), 7.445 (5.59), 7.461 (5.04), 7.465 (4.82), 8.021 (5.32), 12.589 (0.72); LC-MS (method 3): Rₜ=1.20 min; MS (ESIpos): m/z=433 [M+H]⁺

### Example 173

### 6-(Azetidin-1-yl)-4-fluoro-N-(7-methylquinoline-8-sulfonyl)-1-benzofuran-2-carboxamide

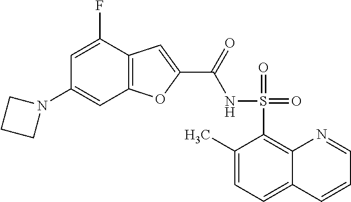 [SMILES: Cc1ccc2cccnc2c1S(=O)(=O)NC(=O)c1cc2c(F)cc(N3CCC3)cc2o1]

Synthesized and purified analogously to Example 172 using 7-methylquinoline-8-sulfonyl chloride (99.1 mg, 410 μmol) instead of 2,3-dihydro-1,4-benzodioxine-5-sulfonyl chloride to give 4.00 mg (3% yield) of the title compound as an orange solid. ¹H-NMR (400 MHz, DMSO-d₆) δ [ppm]: 2.074 (0.40), 2.286 (1.38), 2.304 (2.04), 2.323 (2.21), 2.523 (3.53), 2.669 (0.99), 2.944 (16.00), 3.841 (4.17), 3.859 (6.95), 3.877 (4.01), 6.272 (1.78), 6.291 (3.52), 6.298 (2.28), 7.710 (2.59), 7.721 (1.24), 7.731 (2.69), 7.742 (1.08), 7.849 (1.45), 8.239 (1.55), 8.260 (1.43), 8.642 (1.08), 8.660 (1.01), 9.114 (1.44), 9.121 (1.44); LC-MS (method 3): Rₜ=1.06 min; MS (ESIpos): m/z=440 [M+H]⁺

### Example 174

### 6-(Azetidin-1-yl)-N-(4-bromo-2-methoxybenzene-1-sulfonyl)-4-fluoro-1-benzofuran-2-carboxamide

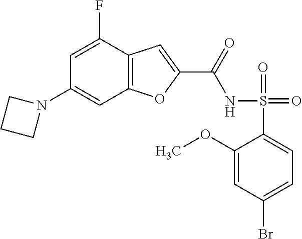 [SMILES: COc1cc(Br)ccc1S(=O)(=O)NC(=O)c1cc2c(F)cc(N3CCC3)cc2o1]

Synthesized and purified analogously to Example 172 using 4-bromo-2-methoxybenzene-1-sulfonyl chloride (117 mg, 410 μmol) instead of 2,3-dihydro-1,4-benzodioxine-5-sulfonyl chloride to give 10.0 mg (6% yield) of the title compound as a green solid. ¹H-NMR (400 MHz, DMSO-d₆) δ [ppm]: 2.306 (1.18), 2.325 (1.82), 2.344 (1.33), 2.361 (0.44), 2.522 (1.05), 2.539 (0.99), 3.873 (3.47), 3.892 (6.24), 3.901 (16.00), 3.910 (3.96), 6.315 (1.29), 6.319 (1.52), 6.352 (3.38), 7.365 (1.71), 7.369 (1.84), 7.386 (1.79), 7.391 (1.98), 7.474 (3.02), 7.478 (2.91), 7.811 (4.08), 7.832 (3.51), 8.014 (2.49); LC-MS (method 3): Rₜ=1.35 min; MS (ESIpos): m/z=485 [M+H]⁺

### Example 175

### 6-(Azetidin-1-yl)-4-fluoro-N-(6-methylquinoline-8-sulfonyl)-1-benzofuran-2-carboxamide

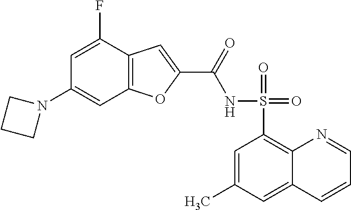 [SMILES: Cc1cc(S(=O)(=O)NC(=O)c2cc3c(F)cc(N4CCC4)cc3o2)c2ncccc2c1]

Synthesized and purified analogously to Example 172 using 6-methylquinoline-8-sulfonyl chloride (99.1 mg, 410 μmol) instead of 2,3-dihydro-1,4-benzodioxine-5-sulfonyl chloride to give 7.00 mg (4% yield) of the title compound as a light yellow solid. ¹H-NMR (400 MHz, DMSO-d₆) δ [ppm]: 2.074 (0.65), 2.153 (0.57), 2.287 (2.04), 2.306 (2.96), 2.324 (2.99), 2.621 (16.00), 2.669 (1.12), 3.847 (5.63), 3.865 (9.63), 3.884 (5.43), 6.293 (8.75), 6.322 (2.42), 7.611 (2.13), 7.622 (2.17), 7.632 (2.20), 7.643 (2.15), 8.071 (2.69), 8.144 (3.59), 8.370 (4.24), 8.374 (4.04), 8.429 (2.36), 8.432 (2.37), 8.449 (2.32), 8.961 (2.57), 8.965 (2.63), 8.972 (2.57); LC-MS (method 3): Rₜ=1.22 min; MS (ESIpos): m/z=440 [M+H]⁺

### Example 176

### 6-(Azetidin-1-yl)-4-fluoro-N-(1H-indole-3-sulfonyl)-1-benzofuran-2-carboxamide

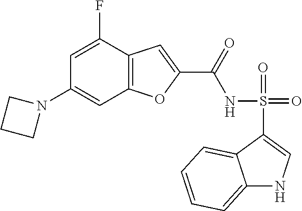 [SMILES: O=C(NS(=O)(=O)c1c[nH]c2ccccc12)c1cc2c(F)cc(N3CCC3)cc2o1]

Synthesized and purified analogously to Example 172 using 1-(4-methylbenzene-1-sulfonyl)-1H-indole-3-sulfonyl chloride (152 mg, 410 μmol) instead of 2,3-dihydro-1,4-benzodioxine-5-sulfonyl chloride to give 3.00 mg (2% yield) of the title compound as yellow solid. ¹H-NMR (400 MHz, DMSO-d₆) δ [ppm]: 2.154 (2.54.) 2.2.294 (2.94), 2.313 (4.35), 2.331 (5.71), 2.424 (0.78), 2.523 (12.09), 2.539 (6.00), 2.602 (1.07), 2.669 (3.56), 3.856 (8.45), 3.875 (14.42), 3.893 (8.27), 6.292 (4.07), 6.319 (16.00), 7.200 (1.59), 7.214 (4.28), 7.217 (4.04), 7.231 (6.09), 7.236 (6.28), 7.250 (3.67), 7.254 (4.17), 7.268 (1.94), 7.507 (4.78), 7.524 (4.34), 7.736 (0.86), 7.877 (3.72), 7.894 (14.24), 8.174 (8.24), 8.182 (8.13), 9.160 (0.81), 12.230 (3.32), 12.385 (3.45); LC-MS (method 3): Rₜ=1.18 min; MS (ESIpos): m/z=414 [M+H]⁺

### Example 177

### 6-(Azetidin-1-yl)-N-(5-bromo-2,3-dihydro-1-benzofuran-7-sulfonyl)-4-fluoro-1-benzofuran-2-carboxamide

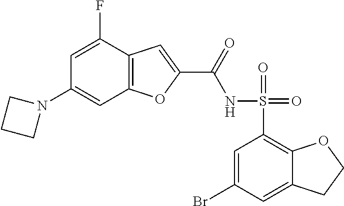 [SMILES: O=C(NS(=O)(=O)c1cc(Br)cc2c1OCC2)c1cc2c(F)cc(N3CCC3)cc2o1]

Synthesized and purified analogously to Example 172 using 5-bromo-2,3-dihydro-1-benzofuran-7-sulfonyl chloride (122 mg, 410 μmol) instead of 2,3-dihydro-1,4-benzodioxine-5-sulfonyl chloride to give 7.00 mg (4% yield) of the title compound as a light yellow solid. ¹H-NMR (400 MHz, DMSO-de) b [ppm]: 1.382 (0.57), 2.074 (0.83), 2.292 (1.14), 2.310 (3.26), 2.328 (5.60), 2.347 (3.61), 2.365 (1.24), 2.466 (0.72), 2.472 (1.14), 2.477 (1.79), 2.531 (1.49), 2.539 (2.20), 2.669 (1.31), 3.234 (3.43), 3.256 (6.75), 3.278 (4.15), 3.879 (9.54), 3.898 (16.00), 3.916 (9.15), 4.679 (4.25), 4.701 (8.21), 4.722 (4.04), 6.318 (3.55), 6.322 (4.16), 6.354 (9.35), 7.664 (6.20), 7.669 (7.07), 7.737 (5.58), 7.962 (3.61); LC-MS (method 3): Rₜ=1.34 min; MS (ESIpos): m/z=497 [M+H]⁺

### Example 178

### 6-(Azetidin-1-yl)-N-(1-benzothiophene-3-sulfonyl)-4-fluoro-1-benzofuran-2-carboxamide

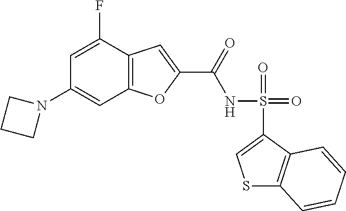 [SMILES: O=C(NS(=O)(=O)c1csc2ccccc12)c1cc2c(F)cc(N3CCC3)cc2o1]

Synthesized and purified analogously to Example 172 using 1-benzothiophene-3-sulfonyl chloride (95.4 mg, 410 μmol) instead of 2,3-dihydro-1,4-benzodioxine-5-sulfonyl chloride to give 7.00 mg (5% yield) of the title compound as a green solid. ¹H-NMR (400 MHz, DMSO-d₆) δ [ppm]: 2.276 (1.03), 2.294 (3.24), 2.312 (4.72), 2.331 (4.04), 2.349 (1.20), 2.539 (1.22), 2.669 (0.63), 3.860 (9.48), 3.878 (16.00), 3.896 (9.28), 6.296 (3.28), 6.300 (4.34), 6.325 (11.33), 7.486 (1.77), 7.503 (4.12), 7.521 (3.24), 7.524 (3.02), 7.545 (3.15), 7.549 (3.52), 7.566 (4.57), 7.569 (3.88), 7.584 (2.09), 7.586 (1.93), 7.911 (7.30), 8.144 (5.05), 8.163 (4.88), 8.243 (4.86), 8.262 (4.36), 8.833 (9.59); LC-MS (method 3): Rₜ=1.38 min; MS (ESIpos): m/z=431 [M+H]⁺

### Example 179

### 6-(Azetidin-1-yl)-4-fluoro-N-(2-methyl-2H-benzotriazole-4-sulfonyl)-1-benzofuran-2-carboxamide

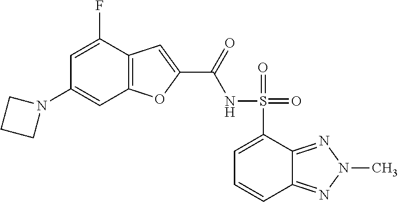 [SMILES: Cn1nc2cccc(S(=O)(=O)NC(=O)c3cc4c(F)cc(N5CCC5)cc4o3)c2n1]

Synthesized and purified analogously to Example 172 using 2-methyl-2H-benzotriazole-4-sulfonyl chloride (94.9 mg, 410 μmol) instead of 2,3-dihydro-1,4-benzodioxine-5-sulfonyl chloride to give 6.00 mg (4% yield) of the title compound as a light yellow solid. ¹H-NMR (400 MHz, DMSO-d₆) δ [ppm]: 2.295 (0.93), 2.314 (1.35), 2.322 (0.56), 2.326 (0.67), 2.332 (1.17), 2.518 (1.07), 2.522 (0.68), 2.539 (0.84), 3.860 (2.86), 3.879 (4.68), 3.897 (2.70), 4.538 (16.00), 6.304 (0.88), 6.308 (1.50), 6.319 (2.25), 6.334 (1.38), 6.338 (0.92), 7.648 (1.48), 7.665 (1.68), 7.668 (1.63), 7.687 (1.65), 8.021 (1.47), 8.137 (1.68), 8.139 (1.74), 8.155 (1.63), 8.157 (1.54), 8.319 (1.63), 8.321 (1.67), 8.340 (1.56), 8.342 (1.45); LC-MS (method 3): Rₜ=1.21 min; MS (ESIpos): m/z=430 [M+H]⁺

### Example 180

### 6-(Azetidin-1-yl)-4-fluoro-N-(4-methoxy-5,6,7,8-tetrahydronaphthalene-1-sulfonyl)-1-benzofuran-2-carboxamide

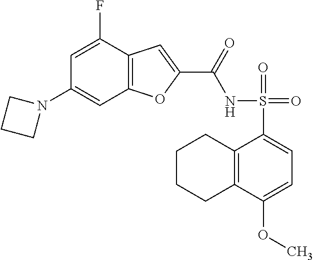 [SMILES: COc1ccc(S(=O)(=O)NC(=O)c2cc3c(F)cc(N4CCC4)cc3o2)c2c1CCCC2]

Synthesized and purified analogously to Example 172 using 4-methoxy-5,6,7,8-tetrahydronaphthalene-1-sulfonyl chloride (107 mg, 410 μmol) instead of2,3-dihydro-1,4-benzodioxine-5-sulfonyl chloride to give 3.00 mg (2% yield) of the title compound as a light yellow solid. ¹H-NMR (400 MHz, DMSO-de) b [ppm]: 1.679 (3.39), 1.688 (2.58), 2.304 (1.14), 2.322 (1.80), 2.341 (1.26), 2.358 (0.40), 2.518 (0.94), 2.522 (0.62), 2.539 (1.51), 2.567 (1.78), 3.055 (1.73), 3.734 (0.71), 3.759 (1.23), 3.862 (16.00), 3.870 (4.19), 3.888 (5.83), 3.906 (3.38), 6.308 (1.39), 6.312 (1.62), 6.337 (1.09), 6.341 (2.07), 6.347 (2.80), 6.349 (3.05), 7.002 (2.31), 7.025 (2.38), 7.929 (2.64), 7.951 (2.83), 7.955 (4.88), 7.957 (4.48); LC-MS (method 3): Rₜ=1.45 min; MS (ESIpos): m/z=459 [M+H]⁺

### Example 181

### 6-(Azetidin-1-yl)-N-(2,1,3-benzothiadiazole-4-sulfonyl)-4-fluoro-1-benzofuran-2-carboxamide

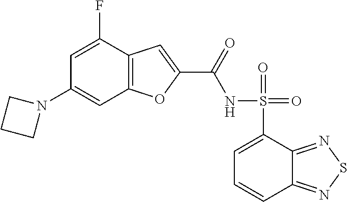 [SMILES: O=C(NS(=O)(=O)c1cccc2nsnc12)c1cc2c(F)cc(N3CCC3)cc2o1]

Synthesized and purified analogously to Example 172 using 2,1,3-benzothiadiazole-4-sulfonyl chloride (120 mg, 512 μmol) instead of 2,3-dihydro-1,4-benzodioxine-5-sulfonyl chloride to give 9.00 mg (5% yield) of the title compound as an orange solid. ¹H-NMR (400 MHz, DMSO-d₆) δ [ppm]: 2.074 (2.08), 2.274 (0.87), 2.292 (2.74), 2.311 (3.96), 2.322 (1.68), 2.329 (3.38), 2.347 (0.98), 2.518 (3.29), 2.522 (2.11), 2.539 (1.28), 2.664 (0.61), 2.669 (0.80), 2.673 (0.59), 3.437 (1.12), 3.855 (8.57), 3.874 (13.92), 3.892 (8.27), 6.303 (16.00), 6.331 (3.97), 6.335 (3.13), 7.938 (4.70), 7.956 (5.49), 7.960 (5.47), 7.978 (5.14), 8.028 (6.00), 8.449 (4.72), 8.452 (6.02), 8.467 (5.33), 8.470 (6.69), 8.471 (7.25), 8.474 (5.50), 8.494 (5.26), 8.496 (4.31); LC-MS (method 3): Rₜ=1.23 min; MS (ESIpos): m/z=433 [M+H]⁺

### Example 182

### 6-(Azetidin-1-yl)-4-fluoro-N-(5-methoxy-1,2,3-benzothiadiazole-4-sulfonyl)-1-benzofuran-2-carboxamide

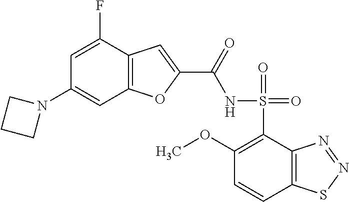 [SMILES: COc1ccc2snnc2c1S(=O)(=O)NC(=O)c1cc2c(F)cc(N3CCC3)cc2o1]

Synthesized and purified analogously to Example 172 using 5-methoxy-1,2,3-benzothiadiazole-4-sulfonyl chloride (108 mg, 410 μmol) instead of 2,3-dihydro-1,4-benzodioxine-5-sulfonyl chloride to give 6.00 mg (4% yield) of the title compound as a yellow solid. ¹H-NMR (400 MHz, DMSO-d₆) δ [ppm]: 2.306 (1.14), 2.326 (1.99), 2.344 (1.27), 2.362 (0.41), 2.518 (1.90), 2.522 (1.23), 2.539 (0.58), 2.668 (0.47), 3.873 (3.56), 3.891 (5.87), 3.910 (3.43), 4.040 (16.00), 6.328 (1.18), 6.332 (1.84), 6.344 (2.80), 6.358 (1.72), 6.363 (1.15), 7.884 (2.84), 7.906 (3.01), 8.053 (1.60), 8.693 (3.36), 8.716 (2.98); LC-MS (method 6): Rₜ=1.15 min; MS (ESIpos): m/z=463 [M+H]⁺

### Example 183

### 6-(Azetidin-1-yl)-N-(3,4-dichloro-2-methoxybenzene-1-sulfonyl)-4-fluoro-1-benzofuran-2-carboxamide

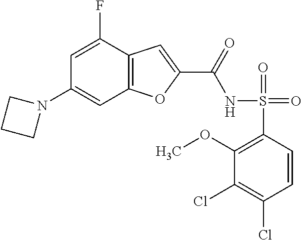 [SMILES: COc1c(S(=O)(=O)NC(=O)c2cc3c(F)cc(N4CCC4)cc3o2)ccc(Cl)c1Cl]

Synthesized and purified analogously to Example 172 using 3,4-dichloro-2-methoxybenzene-1-sulfonyl chloride (113 mg, 410 μmol) instead of 2,3-dihydro-1,4-benzodioxine-5-sulfonyl chloride to give 4.00 mg (90% purity, 2% yield) of the title compound as a yellow solid. ¹H-NMR (400 MHz, DMSO-d6) δ[ppm]: 2.074 (0.88), 2.306 (1.20), 2.326 (2.11), 2.344 (1.32), 2.362 (0.42), 2.518 (1.92), 2.522 (1.23), 2.539 (1.35), 2.668 (0.52), 3.876 (3.71), 3.894 (6.10), 3.912 (3.54), 4.005 (16.00), 6.316 (1.35), 6.319 (1.70), 6.346 (6.12), 7.394 (2.75), 7.418 (2.92), 7.983 (1.33), 8.140 (3.89), 8.162 (3.44); LC-MS (method 3): Rₜ=1.38 min; MS (ESIpos): m/z=473 [M+H]⁺

### Example 184

### 6-(Azetidin-1-yl)-N-(6,7-dihydro-5H-pyrazolo[5,1-b][1,3]oxazine-3-sulfonyl)-4-fluoro-1-benzofuran-2-carboxamide

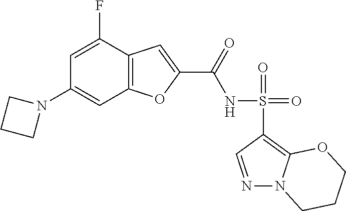 [SMILES: O=C(NS(=O)(=O)c1cnn2c1OCCC2)c1cc2c(F)cc(N3CCC3)cc2o1]

Synthesized and purified analogously to Example 172 using 6,7-dihydro-5H-pyrazolo[5,1-b][1,3]oxazine-3-sulfonyl chloride (114 mg, 512 μmol) instead of 2,3-dihydro-1,4-benzodioxine-5-sulfonyl chloride to give 11.0 mg (6% yield) of the title compound as a yellow solid. ¹H-NMR (400 MHz, DMSO-d₆) δ [ppm]: 2.169 (0.88), 2.183 (2.59), 2.197 (3.22), 2.210 (2.68), 2.224 (0.90), 2.294 (0.83), 2.311 (2.59), 2.330 (4.32), 2.349 (2.87), 2.366 (0.89), 2.518 (4.34), 2.522 (2.82), 2.539 (0.79), 2.664 (0.80), 2.668 (1.11), 2.673 (0.82), 3.877 (8.06), 3.896 (13.17), 3.914 (7.65), 4.084 (3.19), 4.099 (6.76), 4.114 (3.06), 4.423 (3.60), 4.437 (4.56), 4.449 (3.49), 6.309 (3.08), 6.313 (3.50), 6.339 (2.94), 6.342 (3.67), 6.364 (5.24), 6.366 (6.31), 7.700 (16.00), 7.903 (5.06), 12.362 (1.16); LC-MS (method 3): Rₜ=1.05 min; MS (ESIpos): m/z=421 [M+H]⁺

### Example 185

### N-(4-Aacetamidonaphthalene-1-sulfonyl)-6-(azetidin-1-yl)-4-fluoro-1-benzofuran-2-carboxamide

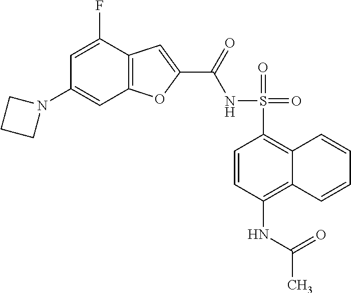 [SMILES: CC(=O)Nc1ccc(S(=O)(=O)NC(=O)c2cc3c(F)cc(N4CCC4)cc3o2)c2ccccc12]

Synthesized and purified analogously to Example 172 using 4-acetamidonaphthalene-1-sulfonyl chloride (116 mg, 410 μmol) instead of 2,3-dihydro-1,4-benzodioxine-5-sulfonyl chloride to give 1.00 mg (1% yield) of the title compound as a yellow solid. ¹H-NMR (400 MHz, DMSO-d₆) δ [ppm]: 2.247 (16.00), 2.272 (0.81), 2.289 (1.74), 2.309 (2.39), 2.326 (3.76), 2.345 (0.86), 2.518 (11.20), 2.522 (7.91), 2.539 (4.21), 2.664 (1.60), 2.668 (2.05), 2.673 (1.57), 3.852 (4.42), 3.870 (7.54), 3.889 (4.53), 6.290 (1.88), 6.310 (4.24), 6.316 (2.56), 7.662 (0.82), 7.680 (1.58), 7.699 (1.29), 7.735 (1.15), 7.754 (1.43), 7.771 (0.79), 7.863 (0.67), 8.041 (1.31), 8.061 (1.48), 8.318 (1.63), 8.340 (3.22), 8.362 (1.79), 8.698 (2.00), 8.720 (1.92), 10.260 (2.47); LC-MS (method 3): Rₜ=1.16 min; MS (ESIpos): m/z=482 [M+H]⁺

### Example 186

### 6-(Azetidin-1-yl)-4-fluoro-N-(imidazo[1,2-a]pyridine-3-sulfonyl)-1-benzofuran-2-carboxamide

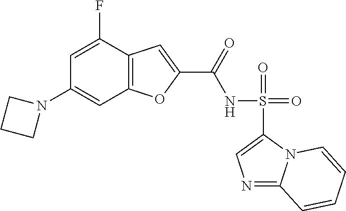 [SMILES: O=C(NS(=O)(=O)c1cnc2ccccn12)c1cc2c(F)cc(N3CCC3)cc2o1]

Synthesized and purified analogously to Example 172 using imidazo[1,2-a]pyridine-3-sulfonyl chloride (88.8 mg, 410 μmol) instead of 2,3-dihydro-1,4-benzodioxine-5-sulfonyl chloride to give 10.0 mg (85% purity, 6% yield) of the title compound as a yellow solid. ¹H-NMR (400 MHz, DMSO-d₆) δ [ppm]: 2.074 (4.30), 2.275 (1.05), 2.293 (3.12), 2.312 (4.67), 2.322 (3.05), 2.331 (5.43), 2.348 (1.38), 2.518 (9.63), 2.522 (6.11), 2.539 (2.50), 2.664 (2.03), 2.668 (2.71), 2.673 (2.02), 2.706 (4.22), 3.846 (9.90), 3.864 (16.00), 3.882 (9.40), 3.924 (1.24), 3.943 (1.68), 3.961 (1.06), 4.355 (0.49), 4.388 (1.00), 4.427 (0.97), 6.254 (3.35), 6.257 (3.64), 6.283 (3.27), 6.287 (3.78), 6.313 (6.15), 6.315 (7.28), 6.351 (0.42), 6.400 (0.55), 7.084 (0.45), 7.427 (1.63), 7.444 (3.34), 7.463 (2.78), 7.544 (0.43), 7.616 (0.70), 7.639 (0.44), 7.765 (1.09), 7.785 (2.00), 7.804 (1.74), 7.881 (5.08), 7.903 (3.30), 8.115 (0.77), 8.395 (6.57), 8.448 (0.66), 8.466 (0.58), 9.006 (2.74), 9.024 (2.69); LC-MS (method 3): Rₜ=0.99 min; MS (ESIpos): m/z=415 [M+H]⁺

### Example 187

### 6-(Azetidin-1-yl)-4-fluoro-N-(isoquinoline-5-sulfonyl)-1-benzofuran-2-carboxamide

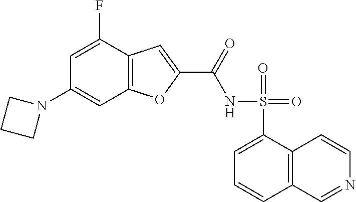 [SMILES: O=C(NS(=O)(=O)c1cccc2cnccc12)c1cc2c(F)cc(N3CCC3)cc2o1]

Synthesized and purified analogously to Example 172 using isoquinoline-5-sulfonyl chloride hydrogen chloride (1/1) (81.2 mg, 307 μmol) instead of 2,3-dihydro-1,4-benzodioxine-5-sulfonyl chloride to give 2.00 mg (2% yield) of the title compound as a yellow solid. ¹H-NMR (400 MHz, DMSO-d₆) δ [ppm]: 2.273 (1.03), 2.290 (2.83), 2.309 (3.91), 2.327 (5.70), 2.345 (1.05), 2.518 (16.00), 2.522 (11.05), 2.539 (4.88), 2.664 (1.93), 2.669 (2.75), 2.673 (2.00), 3.848 (7.74), 3.866 (12.72), 3.885 (7.47), 6.274 (2.58), 6.277 (3.06), 6.307 (11.63), 7.072 (0.46), 7.707 (1.08), 7.909 (2.86), 7.928 (4.45), 7.948 (3.08), 8.132 (0.85), 8.495 (3.12), 8.515 (2.92), 8.580 (4.87), 8.598 (4.19), 8.698 (6.12), 8.713 (4.59), 9.532 (6.49); LC-MS (method 3): Rₜ=0.92 min; MS (ESIpos): m/z=426 [M+H]⁺

### Example 188

### 6-(Azetidin-1-yl)-4-fluoro-N-[5-(morpholin-4-yl)naphthalene-1-sulfonyl]-1-benzofuran-2-carboxamide

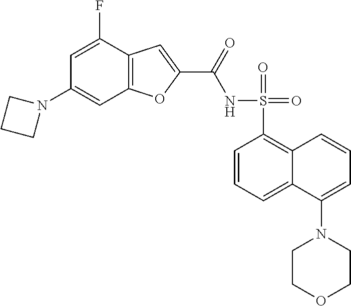 [SMILES: O=C(NS(=O)(=O)c1cccc2c(N3CCOCC3)cccc12)c1cc2c(F)cc(N3CCC3)cc2o1]

Synthesized and purified analogously to Example 172 using 5-(morpholin-4-yl)naphthalene-1-sulfonyl chloride (47.9 mg, 154 μmol) instead of 2,3-dihydro-1,4-benzodioxine-5-sulfonyl chloride to give 8.00 mg (12% yield) of the title compound as a light yellow solid. ¹H-NMR (400 MHz, DMSO-de) b [ppm]: 2.273 (0.70), 2.291 (2.18), 2.310 (3.22), 2.322 (2.60), 2.327 (4.56), 2.345 (0.74), 2.518 (8.74), 2.523 (5.39), 2.539 (4.05), 2.664 (1.71), 2.669 (2.41), 2.673 (1.75), 3.003 (6.16), 3.853 (12.49), 3.864 (10.52), 3.872 (16.00), 3.890 (6.73), 6.292 (2.09), 6.296 (3.43), 6.307 (5.76), 6.322 (3.27), 6.326 (2.30), 7.278 (3.22), 7.296 (3.53), 7.636 (2.05), 7.656 (2.76), 7.676 (1.92), 7.712 (2.35), 7.732 (2.98), 7.751 (2.44), 7.891 (1.20), 8.372 (2.75), 8.390 (5.69), 8.413 (3.00), 8.557 (2.39), 8.578 (2.33); LC-MS (method 3): Rₜ=1.29 min; MS (ESIpos): m/z=510 [M+H]⁺

### Example 189

### 6-(Azetidin-1-yl)-N-(5-tert-butyl-2,3-dimethylbenzene-1-sulfonyl)-4-fluoro-1-benzofuran-2-carboxamide

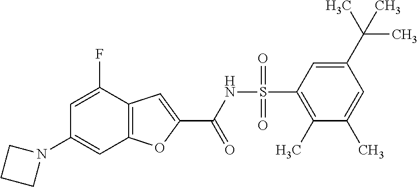 [SMILES: Cc1cc(C(C)(C)C)cc(S(=O)(=O)NC(=O)c2cc3c(F)cc(N4CCC4)cc3o2)c1C]

Synthesized and purified analogously to Example 172 using 5-tert-butyl-2,3-dimethylbenzene-1-sulfonyl chloride (134 mg, 512 μmol) instead of 2,3-dihydro-1,4-benzodioxine-5-sulfonyl chloride to give 12.0 mg (6% yield) of the title compound as a light yellow solid. ¹H-NMR (400 MHz, DMSO-d₆) δ [ppm]: 1.296 (16.00), 2.294 (2.62), 2.325 (0.44), 2.470 (2.80), 3.366 (0.49), 3.874 (0.87), 3.892 (1.43), 3.910 (0.84), 6.318 (0.44), 6.337 (0.74), 6.339 (0.65), 6.344 (0.54), 7.547 (0.49), 7.553 (0.51), 7.902 (0.60), 7.907 (0.60), 7.950 (0.58), LC-MS (method 3): Rₜ=1.50 min; MS (ESIpos): m/z=459 [M+H]⁺

### Example 190

### 6-(Azetidin-1-yl)-N-(3,3-dimethyl-2,3-dihydro-1-benzofuran-5-sulfonyl)-4-fluoro-1-benzofuran-2-carboxamide

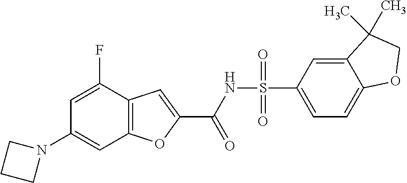 [SMILES: CC1(C)COc2ccc(S(=O)(=O)NC(=O)c3cc4c(F)cc(N5CCC5)cc4o3)cc21]

Synthesized and purified analogously to Example 172 using 3,3-dimethyl-2,3-dihydro-1-benzofuran-5-sulfonyl chloride (126 mg, 512 μmol) instead of 2,3-dihydro-1,4-benzodioxine-5-sulfonyl chloride to give 6.00 mg (3% yield) of the title compound as a yellow solid. ¹H-NMR (400 MHz, DMSO-d₆) δ [ppm]: 1.330 (16.00), 1.436 (0.60), 2.308 (0.84), 2.326 (1.50), 2.345 (0.97), 2.518 (2.09), 2.523 (1.53), 3.874 (2.45), 3.892 (4.11), 3.911 (2.40), 4.364 (6.13), 6.307 (0.95), 6.311 (1.09), 6.336 (0.83), 6.340 (1.24), 6.354 (2.00), 6.983 (2.08), 6.994 (0.50), 7.005 (2.29), 7.783 (0.83), 7.788 (2.13), 7.794 (2.74), 7.799 (2.60), 7.806 (1.97), 7.811 (0.86), 7.863 (1.74), LC-MS (method 3): Rₜ=1.32 min; MS (ESIpos): m/z=445 [M+H]⁺

### Example 191

### 6-(Azetidin-1-yl)-4-fluoro-N-(8-methylisoquinoline-5-sulfonyl)-1-benzofuran-2-carboxamide

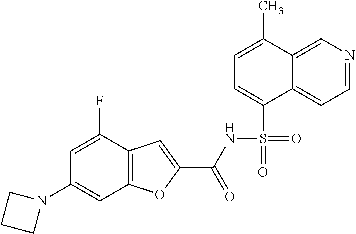 [SMILES: Cc1ccc(S(=O)(=O)NC(=O)c2cc3c(F)cc(N4CCC4)cc3o2)c2ccncc12]

Synthesized and purified analogously to Example 172 using 8-methylisoquinoline-5-sulfonyl chloride (124 mg, 512 μmol) instead of 2,3-dihydro-1,4-benzodioxine-5-sulfonyl chloride to give 11.0 mg (5% yield) of the title compound as an orange solid. ¹H-NMR (400 MHz, DMSO-d₆) δ [ppm]: 2.271 (0.65), 2.289 (1.94), 2.308 (2.75), 2.326 (2.99), 2.344 (0.70), 2.518 (3.61), 2.522 (2.20), 2.539 (1.31), 2.664 (0.70), 2.669 (1.05), 2.673 (1.18), 2.770 (0.55), 2.878 (16.00), 3.408 (0.55), 3.824 (0.50), 3.848 (5.66), 3.866 (9.17), 3.884 (5.46), 6.276 (1.97), 6.280 (2.58), 6.306 (9.05), 7.730 (2.82), 7.732 (2.82), 7.749 (3.93), 7.751 (4.05), 8.478 (4.96), 8.497 (4.54), 8.585 (1.50), 8.599 (1.73), 8.728 (5.94), 8.743 (4.81), 9.642 (5.95), 9.644 (5.87); LC-MS (method 3): Rₜ=0.96 min; MS (ESIpos): m/z=440 [M+H]⁺

### Example 192

### Methyl (5-{[6-(azetidin-1-yl)-4-fluoro-1-benzofuran-2-carbonyl]sulfamoyl}naphthalen-1-yl)carbamate

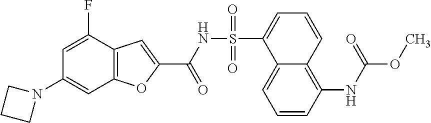 [SMILES: COC(=O)Nc1cccc2c(S(=O)(=O)NC(=O)c3cc4c(F)cc(N5CCC5)cc4o3)cccc12]

Synthesized analogously to Example 172 using Methyl [5-(chlorosulfonyl)naphthalen-1-yl]carbamate (123 mg, 410 μmol) instead of 2,3-dihydro-1,4-benzodioxine-5-sulfonyl chloride and purified by HT-HPLC (basic method) to give 1.00 mg (90% purity, 1% yield) of the title compound as a light yellow solid. ¹H-NMR (400 MHz, METHANOL-d₄) δ [ppm]: 1.290 (1.24), 2.208 (0.57), 2.370 (0.49), 2.389 (1.53), 2.407 (2.25), 2.425 (1.68), 2.444 (0.52), 2.660 (16.00), 3.133 (0.53), 3.340 (1.21), 3.482 (0.48), 3.784 (9.84), 3.839 (0.42), 3.915 (4.63), 3.933 (7.44), 3.951 (4.39), 6.164 (1.56), 6.169 (1.64), 6.193 (1.56), 6.197 (1.63), 6.312 (2.80), 7.459 (2.89), 7.644 (0.95), 7.663 (1.87), 7.675 (1.63), 7.685 (1.88), 7.693 (1.93), 7.696 (2.02), 7.715 (2.40), 7.730 (0.65), 8.372 (1.39), 8.394 (1.31), 8.489 (1.68), 8.491 (1.71), 8.507 (1.67), 8.509 (1.59), 8.668 (1.36), 8.689 (1.29); LC-MS (method 2): Rₜ=0.73 min; MS (ESIpos): m/z=498 [M+H]⁺

### Example 193

### 6-(Azetidin-1-yl)-4-fluoro-N-(4-methylisoquinoline-5-sulfonyl)-1-benzofuran-2-carboxamide

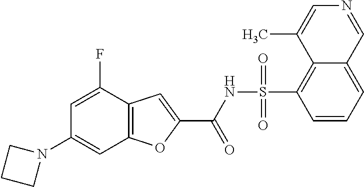 [SMILES: Cc1cncc2cccc(S(=O)(=O)NC(=O)c3cc4c(F)cc(N5CCC5)cc4o3)c12]

Synthesized and purified analogously to Example 192 using 4-Methylisoquinoline-5-sulfonyl chloride (99.1 mg, 410 μmol) instead of Methyl [5-(chlorosulfonyl)naphthalen-1-yl]carbamate to give 1.00 mg (1% yield) of the title compound as a light yellow solid. ¹H-NMR (400 MHz, METHANOL-d₄) δ [ppm]: 0.901 (0.55), 0.998 (0.89), 1.016 (0.50), 1.289 (2.16), 1.401 (0.94), 1.414 (0.69), 1.485 (1.58), 1.501 (1.57), 2.209 (1.78), 2.369 (0.77), 2.387 (2.26), 2.406 (3.31), 2.424 (2.46), 2.442 (0.81), 2.660 (16.00), 2.812 (0.49), 2.888 (0.52), 3.125 (0.40), 3.129 (0.73), 3.133 (1.21), 3.137 (0.75), 3.235 (14.40), 3.252 (1.58), 3.474 (0.40), 3.478 (0.75), 3.482 (1.14), 3.487 (0.72), 3.625 (0.58), 3.910 (6.85), 3.929 (11.00), 3.946 (6.34), 6.169 (2.34), 6.173 (2.45), 6.198 (2.33), 6.202 (2.48), 6.332 (4.04), 7.431 (4.70), 7.803 (2.10), 7.822 (3.76), 7.842 (2.31), 8.359 (2.40), 8.362 (2.39), 8.379 (2.28), 8.382 (2.16), 8.423 (2.98), 8.926 (2.50), 8.930 (2.49), 8.945 (2.47), 8.948 (2.26), 9.208 (2.85), 9.767 (0.49), 9.791 (0.41); LC-MS (method 2): Rₜ=0.71 min; MS (ESIpos): m/z=440 [M+H]⁺

### Example 194

### 6-(Azetidin-1-yl)-N-[5-(3,5-dimethyl-1,2-oxazol-4-yl)-2-methoxybenzene-1-sulfonyl]-4-fluoro-1-benzofuran-2-carboxamide

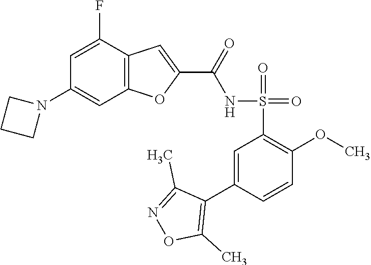 [SMILES: COc1ccc(-c2c(C)noc2C)cc1S(=O)(=O)NC(=O)c1cc2c(F)cc(N3CCC3)cc2o1]

To a solution of 6-(azetidin-1-yl)-N-(5-bromo-2-methoxybenzene-1-sulfonyl)-4-fluoro-1-benzofuran-2-carboxamide (Intermediate 15, 100 mg, 207 μmol) and (3,5-dimethyl-1,2-oxazol-4-yl)boronic acid (29.2 mg, 207 μmol) in a mixture of 1,4-dioxane (6.0 ml) and water (660 μl) was added potassium carbonate (85.8 mg, 621 μmol; CAS-RN:[584-08-7]) and (2′-amino[1,1′-biphenyl]-2-yl)(methanesulfonato-kappaO)palladium-dicyclohexyl(2′,6′-dimethoxy[1,1′-biphenyl]-2-yl)phosphane (1/1) (16.1 mg, 20.7 μmol). The resulting reaction mixture was stirred for at 80° C. for 4 h. work up: The reaction mixture was concentrated in vacuo and the residue was purified by HT-HPLC (acid) to give 20.0 mg (17% yield) of the title compound as an offwhite solid. ¹H-NMR (400 MHz, DMSO-d₆) δ [ppm]: 2.074 (1.04), 2.243 (15.88), 2.307 (1.01), 2.326 (2.26), 2.336 (0.67), 2.345 (1.12), 2.428 (16.00), 2.518 (3.21), 2.522 (2.07), 2.664 (0.61), 2.668 (0.83), 2.673 (0.59), 3.863 (0.70), 3.872 (3.12), 3.891 (5.11), 3.909 (3.17), 3.921 (11.49), 6.317 (1.08), 6.321 (1.25), 6.351 (2.15), 6.354 (2.84), 6.356 (2.93), 7.328 (1.48), 7.350 (1.63), 7.692 (0.81), 7.697 (0.87), 7.713 (0.72), 7.719 (0.76), 7.828 (3.25), 7.834 (2.95), 8.026 (0.53); LC-MS (method 7): Rₜ=1.20 min; MS (ESIpos): m/z=500 [M+H]⁺

### Example 195

### 6-(Azetidin-1-yl)-N-[5-(1,3-dimethyl-1H-pyrazol-4-yl)-2-methoxybenzene-1-sulfonyl]-4-fluoro-1-benzofuran-2-carboxamide

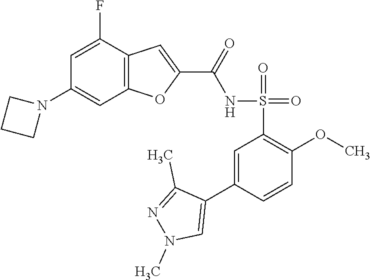 [SMILES: COc1ccc(-c2cn(C)nc2C)cc1S(=O)(=O)NC(=O)c1cc2c(F)cc(N3CCC3)cc2o1]

Synthesized and purified analogously to Example 194 using 1,3-dimethyl-4-(4,4,5,5-tetramethyl-1,3,2-dioxaborolan-2-yl)-1H-pyrazole (46.0 mg, 207 μmol) instead of (3,5-dimethyl-1,2-oxazol-4-yl)boronic acid to give 23.0 mg (21% yield) of the title compound as an offwhite solid. ¹H-NMR (400 MHz, DMSO-d₆) δ [ppm]: 2.075 (1.16), 2.288 (16.00), 2.306 (1.17), 2.323 (2.04), 2.327 (2.15), 2.332 (1.12), 2.337 (0.77), 2.343 (1.20), 2.518 (3.70), 2.523 (2.42), 2.540 (0.60), 2.665 (0.64), 2.669 (0.88), 2.674 (0.61), 3.795 (15.24), 3.877 (13.54), 3.890 (5.66), 3.908 (3.16), 6.316 (1.17), 6.320 (1.42), 6.349 (5.29), 7.247 (1.58), 7.270 (1.70), 7.676 (0.91), 7.682 (0.96), 7.698 (0.82), 7.703 (0.84), 7.885 (3.70), 7.891 (3.71), 7.951 (4.19), 8.030 (0.62); LC-MS (method 7): Rₜ=1.12 min; MS (ESIpos): m/z=499 [M+H]⁺

### Example 196

### 6-(azetidin-1-yl)-N-[5-(3,6-dihydro-2H-pyran-4-yl)-2-methoxybenzene-1-sulfonyl]-4-fluoro-1-benzofuran-2-carboxamide

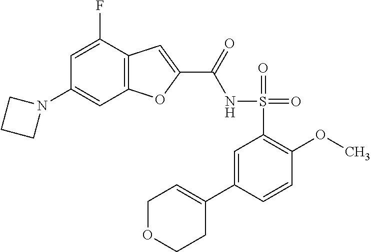 [SMILES: COc1ccc(C2=CCOCC2)cc1S(=O)(=O)NC(=O)c1cc2c(F)cc(N3CCC3)cc2o1]

Synthesized and purified analogously to Example 194 using 4-(4,4,5,5-tetramethyl-1,3,2-dioxaborolan-2-yl)-3,6-dihydro-2H-pyran (43.5 mg, 207 μmol) instead of (3,5-dimethyl-1,2-oxazol-4-yl)boronic acid to give 8.00 mg (8% yield) of the title compound as an offwhite solid. ¹H-NMR (400 MHz, DMSO-d₆) δ [ppm]: 2.305 (1.11), 2.323 (1.85), 2.326 (1.74), 2.331 (0.85), 2.343 (1.21), 2.445 (1.53), 2.449 (1.53), 2.518 (2.52), 2.523 (1.77), 2.669 (0.48), 3.826 (1.78), 3.839 (3.70), 3.853 (1.88), 3.872 (16.00), 3.890 (5.57), 3.908 (3.14), 4.227 (1.11), 4.234 (2.67), 4.241 (2.65), 4.248 (1.12), 6.258 (0.85), 6.262 (1.18), 6.265 (1.54), 6.269 (1.18), 6.315 (1.21), 6.319 (1.44), 6.347 (5.92), 7.214 (1.92), 7.236 (2.07), 7.740 (1.07), 7.747 (1.07), 7.762 (0.96), 7.768 (1.00), 7.906 (2.81), 7.912 (2.60), 8.026 (1.16); LC-MS (method 7): Rₜ=1.19 min; MS (ESIpos): m/z=487 [M+H]⁺

### Example 197

### tert-butyl 4-(3-{[6-(azetidin-1-yl)-4-fluoro-1-benzofuran-2-carbonyl]sulfamoyl}-4-methoxyphenyl)-3,6-dihydropyridine-1(2H)-carboxylate

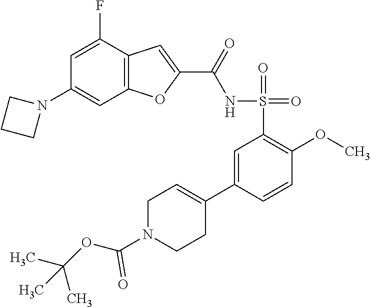 [SMILES: COc1ccc(C2=CCN(C(=O)OC(C)(C)C)CC2)cc1S(=O)(=O)NC(=O)c1cc2c(F)cc(N3CCC3)cc2o1]

Synthesized and purified analogously to Example 194 using tert-butyl 4-(4,4,5,5-tetramethyl-1,3,2-dioxaborolan-2-yl)-3,6-dihydropyridine-1(2H)-carboxylate (128 mg, 414 μmol) instead of (3,5-dimethyl-1,2-oxazol-4-yl)boronic acid to give 5.00 mg (95% purity, 2% yield) of the title compound as an offwhite solid. ¹H-NMR (400 MHz, DMSO-d₆) δ [ppm]: 1.433 (16.00), 2.304 (0.44), 2.323 (0.73), 2.342 (0.49), 2.466 (0.63), 2.523 (0.54), 3.544 (0.44), 3.558 (0.80), 3.572 (0.43), 3.868 (5.60), 3.889 (2.15), 3.907 (1.21), 4.016 (0.70), 6.312 (0.49), 6.316 (0.56), 6.344 (2.32), 7.207 (0.69), 7.229 (0.74), 7.899 (1.03), 7.904 (0.94), LC-MS (method 7): Rₜ=1.41 min; MS (ESIpos): m/z=530 [M+H]⁺

### Example 198

### 6-(Azetidin-1-yl)-4-fluoro-N-(3′-fluoro-4-methoxy[1,1′-biphenyl]-3-sulfonyl)-1-benzofuran-2-carboxamide

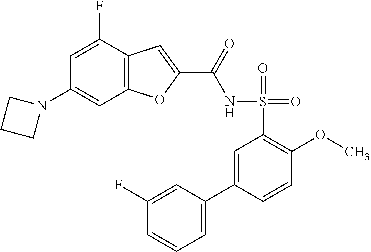 [SMILES: COc1ccc(-c2cccc(F)c2)cc1S(=O)(=O)NC(=O)c1cc2c(F)cc(N3CCC3)cc2o1]

Synthesized and purified analogously to Example 194 using (3-fluorophenyl)boronic acid (20.3 mg, 145 μmol) instead of (3,5-dimethyl-1,2-oxazol-4-yl)boronic acid to give 23.0 mg (30% yield) of the title compound as an offwhite solid. ¹H-NMR (400 MHz, DMSO-d₆) δ [ppm]: 2.074 (0.97), 2.285 (0.43), 2.303 (1.32), 2.322 (2.18), 2.331 (0.95), 2.340 (1.51), 2.357 (0.50), 2.518 (2.77), 2.523 (1.96), 2.669 (0.50), 3.869 (3.88), 3.887 (6.52), 3.906 (3.82), 3.926 (16.00), 6.314 (1.43), 6.318 (1.80), 6.344 (5.85), 7.206 (0.42), 7.212 (0.66), 7.218 (0.54), 7.224 (0.52), 7.229 (1.25), 7.235 (1.00), 7.251 (0.69), 7.257 (0.57), 7.338 (2.30), 7.359 (2.47), 7.511 (1.58), 7.524 (3.21), 7.530 (1.84), 7.536 (2.61), 7.540 (2.60), 7.555 (1.33), 7.561 (0.49), 7.575 (0.45), 8.021 (2.03), 8.026 (2.46), 8.042 (1.47), 8.048 (1.49), 8.127 (4.24), 8.133 (3.76); LC-MS (method 7): Rₜ=1.35 min; MS (ESIpos): m/z=499 [M+H]⁺

### Example 199

### 6-(Azetidin-1-yl)-4-fluoro-N-[2-methoxy-5-(1-methyl-1H-pyrazol-4-yl)benzene-1-sulfonyl]-1-benzofuran-2-carboxamide

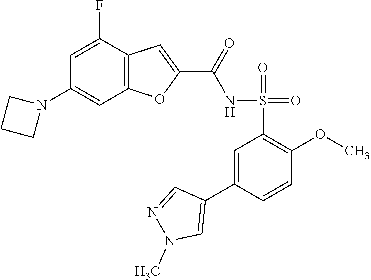 [SMILES: COc1ccc(-c2cnn(C)c2)cc1S(=O)(=O)NC(=O)c1cc2c(F)cc(N3CCC3)cc2o1]

Synthesized and purified analogously to Example 194 using 1-methyl-4-(4,4,5,5-tetramethyl-1,3,2-dioxaborolan-2-yl)-1H-pyrazole (43.1 mg, 207 μmol) instead of (3,5-dimethyl-1,2-oxazol-4-yl)boronic acid to give 15.0 mg (14% yield) of the title compound as a light yellow solid. ¹H-NMR (400 MHz, DMSO-d₆) δ [ppm]: 2.303 (0.86), 2.322 (1.81), 2.326 (1.47), 2.332 (0.95), 2.340 (0.97), 2.518 (3.40), 2.522 (2.18), 2.539 (0.72), 2.664 (0.60), 2.668 (0.83), 2.673 (0.60), 3.865 (11.19), 3.870 (16.00), 3.887 (4.42), 3.905 (2.48), 6.314 (0.93), 6.318 (1.11), 6.348 (3.34), 7.225 (1.21), 7.247 (1.31), 7.834 (0.71), 7.840 (0.74), 7.860 (3.70), 7.982 (2.94), 7.988 (2.71), 8.033 (0.46), 8.208 (2.75), LC-MS (method 7): Rₜ=1.10 min; MS (ESIpos): m/z=485 [M+H]⁺

### Example 200

### 6-(Azetidin-1-yl)-4-fluoro-N-[2-methoxy-5-(pyridin-3-yl)benzene-1-sulfonyl]-1-benzofuran-2-carboxamide

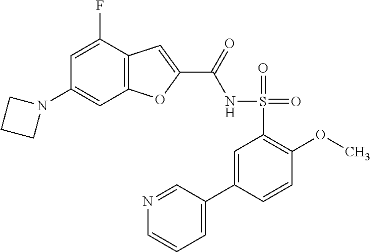 [SMILES: COc1ccc(-c2cccnc2)cc1S(=O)(=O)NC(=O)c1cc2c(F)cc(N3CCC3)cc2o1]

Synthesized and purified analogously to Example 194 using pyridin-3-ylboronic acid (12.7 mg, 103 μmol) instead of (3,5-dimethyl-1,2-oxazol-4-yl)boronic acid to give 9.00 mg (17% yield) of the title compound as a yellow solid. ¹H-NMR (400 MHz, DMSO-d₆) δ [ppm]: 2.074 (4.02), 2.285 (0.64), 2.303 (1.94), 2.322 (3.32), 2.339 (2.23), 2.357 (0.77), 2.539 (9.72), 2.669 (0.89), 3.867 (4.82), 3.885 (8.44), 3.903 (4.96), 3.925 (16.00), 6.312 (2.08), 6.341 (7.69), 7.360 (2.38), 7.381 (2.53), 7.511 (1.32), 7.523 (1.50), 7.530 (1.55), 7.542 (1.45), 7.967 (1.16), 8.042 (1.68), 8.063 (1.63), 8.085 (1.92), 8.104 (1.79), 8.141 (4.10), 8.147 (3.76), 8.606 (1.54), 8.894 (1.93); LC-MS (method 7): Rₜ=0.91 min; MS (ESIpos): m/z=482 [M+H]⁺

### Example 201

### 6-(Azetidin-1-yl)-4-fluoro-N-[2-methoxy-5-(2-phenylpyrrolidin-1-yl)benzene-1-sulfonyl]-1-benzofuran-2-carboxamide (rac)

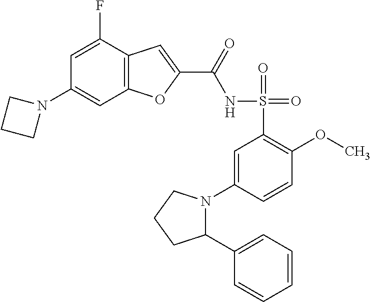 [SMILES: COc1ccc(N2CCCC2c2ccccc2)cc1S(=O)(=O)NC(=O)c1cc2c(F)cc(N3CCC3)cc2o1]

To a pre-activated mixture of di-mu-chloro[bis(1-phenylprop-2-en-1-yl)]dipalladium (10.7 mg, 20.7 μmol; CAS-RN:[12131-44-1]) and di-tert-butyl(1-methyl-2,2-diphenylcyclopropyl)phosphine (14.6 mg, 41.4 μmol; CAS-RN:[742103-27-1]) was added degassed 2-methylbutan-2-ol (3.0 mL, 28 mmol; CAS-RN:[75-85-4]) following by the subsequent addition of 6-(azetidin-1-yl)-N-(5-bromo-2-methoxybenzene-1-sulfonyl)-4-fluoro-1-benzofuran-2-carboxamide (Example 139, 100 mg, 207 μmol), 2-phenylpyrrolidine (34 μL, 230 μmol; CAS-RN:[1006-64-0]) and sodium phenoxide (72.1 mg, 621 μmol; CAS-RN:[139-02-6]) and heating at 100° C. overnight. The mixture was diluted with 1 mL DMF, filtrated and purified by preparative HPLC (water+0.1% HCOOH/acetonitrile). The resulting yellow solid was purified a second time by preparative HPLC (water+0.2% ammonium hydroxide/acetonitrile) to give 10.0 mg (9% yield) of the title compound as a yellow solid. ¹H-NMR (400 MHz, DMSO-d₆) δ [ppm]: 1.137 (3.29), 1.231 (0.78), 1.804 (0.77), 1.820 (0.77), 1.976 (1.62), 1.994 (1.48), 2.083 (16.00), 2.115 (1.45), 2.288 (0.59), 2.306 (1.78), 2.326 (3.79), 2.344 (2.01), 2.362 (0.70), 2.393 (0.72), 2.422 (0.91), 2.443 (0.63), 2.522 (7.18), 2.665 (1.10), 2.668 (1.50), 2.673 (1.12), 3.674 (11.44), 3.714 (1.15), 3.872 (4.56), 3.890 (7.84), 3.908 (4.42), 4.701 (1.27), 4.717 (1.32), 6.310 (1.51), 6.340 (1.75), 6.350 (4.66), 6.607 (0.75), 6.982 (3.71), 6.989 (3.46), 7.189 (0.84), 7.210 (4.94), 7.228 (6.71), 7.283 (3.63), 7.301 (3.83), 7.320 (1.64), 12.359 (0.90); LC-MS (method 2): Rₜ=0.93 min; MS (ESIpos): m/z=550 [M+H]⁺

### Example 202

### 6-(Azetidin-1-yl)-4-fluoro-N-[2-methoxy-5-(4-methylpiperazin-1-yl)benzene-1-sulfonyl]-1-benzofuran-2-carboxamide

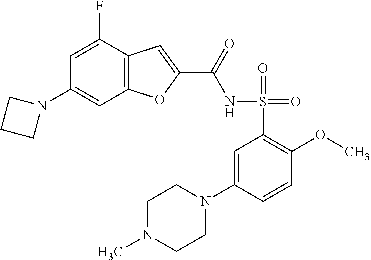 [SMILES: COc1ccc(N2CCN(C)CC2)cc1S(=O)(=O)NC(=O)c1cc2c(F)cc(N3CCC3)cc2o1]

Synthesized and purified analogously to Example 127 using 2-methoxy-5-(4-methylpiperazin-1-yl)benzene-1-sulfonamide (Intermediate 27, 64.0 mg, 224 μmol) instead of 5-tert-butyl-2-methoxybenzene-1-sulfonamide to give 48.0 mg (49% yield) of the title compound as a yellow solid. ¹H-NMR (400 MHz, DMSO-d₆) δ [ppm]: 1.232 (0.90), 1.907 (0.54), 2.273 (0.64), 2.291 (1.97), 2.309 (2.79), 2.327 (3.15), 2.345 (0.71), 2.517 (5.17), 2.522 (3.19), 2.664 (0.96), 2.668 (1.29), 2.673 (0.98), 2.738 (3.02), 3.186 (1.96), 3.679 (16.00), 3.829 (5.33), 3.848 (9.12), 3.865 (5.04), 6.213 (1.75), 6.216 (1.81), 6.243 (1.74), 6.246 (1.79), 6.326 (4.55), 6.946 (1.36), 6.969 (1.87), 7.064 (1.18), 7.081 (0.94), 7.450 (4.01), 7.457 (3.82); LC-MS (method 2): Rₜ=0.72 min; MS (ESIpos): m/z=503 [M+H]⁺

### Example 203

### 6-(Azetidin-1-yl)-4-fluoro-N-[2-methoxy-5-(1-methyl-1,2,3,6-tetrahydropyridin-4-yl)benzene-1-sulfonyl]-1-benzofuran-2-carboxamide

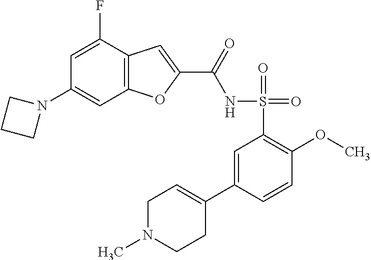 [SMILES: COc1ccc(C2=CCN(C)CC2)cc1S(=O)(=O)NC(=O)c1cc2c(F)cc(N3CCC3)cc2o1]

Synthesized and purified analogously to Example 194 using 1-methyl-4-(4,4,5,5-tetramethyl-1,3,2-dioxaborolan-2-yl)-1,2,3,6-tetrahydropyridine (46.2 mg, 207 μmol) instead of (3,5-dimethyl-1,2-oxazol-4-yl)boronic acid to give 20.0 mg (18% yield) of the title compound as an offwhite solid. ¹H-NMR (400 MHz, DMSO-d₆) δ [ppm]: 2.271 (0.57), 2.290 (1.81), 2.308 (2.55), 2.327 (2.92), 2.336 (0.61), 2.344 (0.64), 2.518 (3.19), 2.523 (2.10), 2.665 (0.72), 2.669 (1.01), 2.674 (0.71), 2.732 (2.16), 2.850 (9.76), 3.743 (16.00), 3.824 (6.37), 3.842 (9.22), 3.860 (5.30), 4.298 (0.58), 6.087 (1.93), 6.201 (1.82), 6.205 (1.90), 6.230 (1.82), 6.234 (1.90), 6.320 (3.60), 6.323 (4.45), 6.326 (3.30), 7.010 (2.05), 7.033 (2.00), 7.508 (1.06), 7.525 (0.96), 7.925 (4.47), 7.932 (4.41), 8.134 (2.21); LC-MS (method 7): Rₜ=0.76 min; MS (ESIpos): m/z=500 [M+H]⁺

### Example 204

### 6-(Azetidin-1-yl)-4-fluoro-N-(2,2,5,6-tetramethyl-2,3-dihydro-1-benzofuran-7-sulfonyl)-1-benzofuran-2-carboxamide

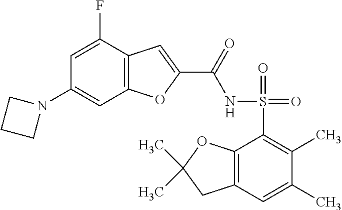 [SMILES: Cc1cc2c(c(S(=O)(=O)NC(=O)c3cc4c(F)cc(N5CCC5)cc4o3)c1C)OC(C)(C)C2]

Dichloromethane (2.0 mL) was added to a Vial containing DMAP (2.60 mg, 21.3 μmol; CAS-RN:[1122-58-3]), 2-chloro-1-methylpyridinium iodide (130 mg, 510 μmol; CAS-RN:[14338-32-0]), 6-(azetidin-1-yl)-4-fluoro-1-benzofuran-2-carboxylic acid

Intermediate 3, 100 mg, 425 μmol) and 2,2,5,6-tetramethyl-2,3-dihydro-1-benzofuran-7-sulfonamide (Intermediate 28, 217 mg, 850 μmol) at room temperature. After stirring for 5 min, triethylamine (180 μL, 1.3 mmol; CAS-RN:[121-44-8]) was added slowly and the resulting mixture was stirred overnight. The reaction mixture was concentrated in vacuo and the oily residue was purified by preparative HPLC (water+0.1% HCOOH/acetonitrile) to give 6.80 mg (3% yield) of the title compound as a light yellow solid. ¹H-NMR (400 MHz, DMSO-d₆) δ [ppm]: 1.172 (15.88), 2.084 (16.00), 2.201 (6.64), 2.309 (0.99), 2.327 (1.89), 2.347 (1.04), 2.467 (7.29), 2.518 (2.39), 2.522 (1.51), 2.539 (4.11), 2.664 (0.44), 2.669 (0.60), 2.673 (0.44), 2.901 (3.09), 3.879 (2.55), 3.897 (4.28), 3.915 (2.37), 6.317 (0.80), 6.347 (0.82), 6.383 (2.20), 7.232 (0.86), 12.319 (0.67); LC-MS (method 1): Rₜ=1.41 min; MS (ESIpos): m/z=473 [M+H]⁺

### Example 205

### 6-(Azetidin-1-yl)-4-fluoro-N-[2-methoxy-5-(prop-1-en-2-yl)benzene-1-sulfonyl]-1-benzofuran-2-carboxamide

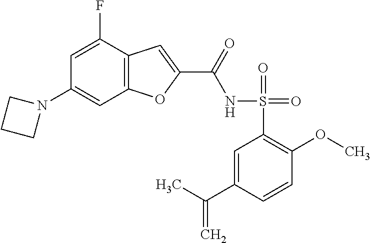 [SMILES: C=C(C)c1ccc(OC)c(S(=O)(=O)NC(=O)c2cc3c(F)cc(N4CCC4)cc3o2)c1]

Synthesized and purified analogously to Example 194 using 4,4,5,5-tetramethyl-2-(prop-1-en-2-yl)-1,3,2-dioxaborolane (69.5 mg, 414 μmol) instead of (3,5-dimethyl-1,2-oxazol-4-yl)boronic acid to give 43 mg (21% yield) of the title compound as an brownish solid. ¹H-NMR (400 MHz, DMSO-d₆) δ [ppm]: 1.846 (0.72), 1.850 (0.74), 1.863 (0.78), 1.867 (0.77), 2.131 (9.05), 2.288 (0.56), 2.306 (1.60), 2.323 (2.90), 2.326 (3.19), 2.331 (1.57), 2.336 (1.05), 2.343 (1.74), 2.361 (0.54), 2.518 (4.31), 2.522 (2.69), 2.660 (0.40), 2.664 (0.93), 2.668 (1.26), 2.673 (0.89), 3.852 (3.17), 3.858 (1.55), 3.876 (16.00), 3.890 (8.01), 3.908 (4.49), 5.142 (1.71), 5.145 (2.44), 5.148 (1.72), 5.428 (2.78), 6.314 (1.57), 6.319 (1.80), 6.346 (7.07), 7.188 (0.44), 7.209 (1.91), 7.230 (2.02), 7.801 (1.03), 7.807 (1.08), 7.823 (1.00), 7.829 (1.22), 7.837 (0.68), 7.933 (0.50), 7.939 (0.46), 7.956 (3.89), 7.962 (3.69), 8.022 (0.89); LC-MS (method 3): Rₜ=1.37 min; MS (ESIpos): m/z=445 [M+H]⁺

### Example 206

### 6-(azetidin-1-yl)-N-(2-ethoxy-3-fluoro-phenyl)sulfonyl-4-fluoro-benzofuran-2-carboxamide

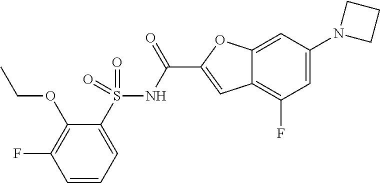 [SMILES: CCOc1c(F)cccc1S(=O)(=O)NC(=O)c1cc2c(F)cc(N3CCC3)cc2o1]

To a room temperature stirred solution of 6-(azetidin-1-yl)-4-fluoro-benzofuran-2-carboxylic acid (94.1 mg, 0.40 mmol, 1.00 eq.) in anhydrous tetrahydrofuran (2.00 mL, 0.20 M) was added carbonyldiimidazole (77.8 mg, 0.48 mmol, 1.20 eq.). After stirring for 1 hour, 2-ethoxy-3-fluoro-benzenesulfonamide (96.5 mg, 0.44 mmol, 1.10 eq.) and 1,8-diazabicyclo[5.4.0]undec-7-ene (80.0 μL, 0.56 mmol, 1.40 eq.) were sequentially added. The resulting mixture was stirred at room temperature for 16 h and then concentrated down under reduced pressure. The residue was purified by reverse phase preparative column chromatography (Waters XBridge PREP C₁₈, 5 μM OBD, 30×150 mm, 57-65% acetonitrile/water gradient buffered with 0.1% formic acid) to give the title compound as a white solid (122 mg). 1H NMR (400 MHz, DMSO-d₆) δ 12.67 (s, 1H), 8.00 (s, 1H), 7.77 (d, 1H), 7.69-7.59 (m, 1H), 7.36-7.26 (m, 1H), 6.37-6.29 (m, 2H), 4.26-4.16 (m, 2H), 3.90 (t, 4H), 2.33 (p, 2H), 1.34 (t, 3H), LC-MS (method 5): Rₜ=3.01 min; MS (ESIpos): m/z=437 [M+H]⁺

### Example 207

### 6-(Azetidin-1-yl)-N-[(5-sec-butyl-2-ethoxyphenyl)sulfonyl]-4-fluoro-1-benzofuran-2-carboxamide (rac)

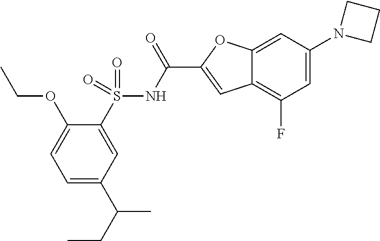 [SMILES: CCOc1ccc(C(C)CC)cc1S(=O)(=O)NC(=O)c1cc2c(F)cc(N3CCC3)cc2o1]

To a room temperature stirred solution of 6-(azetidin-1-yl)-4-fluoro-benzofuran-2-carboxylic acid (94.1 mg, 0.40 mmol, 1.00 eq.) in anhydrous tetrahydrofuran (2.00 mL, 0.20 M) was added carbonyldiimidazole (77.8 mg, 0.48 mmol, 1.20 eq.). After stirring for 1 hour, 2-ethoxy-5-sec-butyl-benzenesulfonamide (113 mg, 0.44 mmol, 1.10 eq.) and 1,8-diazabicyclo[5.4.0]undec-7-ene (80.0 μL, 0.56 mmol, 1.40 eq.) were sequentially added. The resulting mixture was stirred at room temperature for 16 h and then concentrated down under reduced pressure. The residue was purified by reverse phase preparative column chromatography (Waters XBridge PREP C₁₈, 5 μM OBD, 30×150 mm, 66-74% acetonitrile/water gradient buffered with 0.1% formic acid) to give the title compound as a white solid (122 mg). ¹H NMR (400 MHz, DMSO-d₆) δ 12.33 (s, 1H), 8.03 (s, 1H), 7.71-7.66 (m, 1H), 7.52-7.41 (m, 1H), 7.13 (d, 1H), 6.36-6.28 (m, 2H), 4.13 (q, 2H), 3.89 (t, 4H), 2.74-2.60 (m, 1H), 2.33 (p, 2H), 1.64-1.45 (m, 2H), 1.28-1.17 (m, 6H), 0.79 (t, 3H); LC-MS (method 5): Rₜ=3.40 min; MS (ESIpos): m/z=475 [M+H]⁺

### Example 208

### 6-(Azetidin-1-yl)-N-(2-ethoxy-4-fluoro-phenyl)sulfonyl-4-fluoro-benzofuran-2-carboxamide

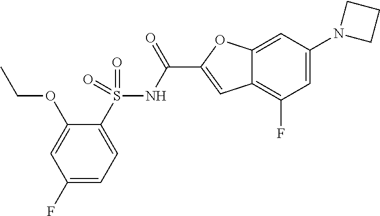 [SMILES: CCOc1cc(F)ccc1S(=O)(=O)NC(=O)c1cc2c(F)cc(N3CCC3)cc2o1]

To a room temperature stirred solution of 6-(azetidin-1-yl)-4-fluoro-benzofuran-2-carboxylic acid (94.1 mg, 0.40 mmol, 1.00 eq.) in anhydrous tetrahydrofuran (2.00 mL, 0.20 M) was added carbonyldiimidazole (77.8 mg, 0.48 mmol, 1.20 eq.). After stirring for 1 hour, 2-ethoxy-4-fluoro-benzenesulfonamide (96.5 mg, 0.44 mmol, 1.10 eq.) and 1,8-diazabicyclo[5.4.0]undec-7-ene (80.0 μL, 0.56 mmol, 1.40 eq.) were sequentially added. The resulting mixture was stirred at room temperature for 16 h and then concentrated down under reduced pressure. The residue was purified by reverse phase preparative column chromatography (Waters XBridge PREP C₁₈, 5 μM OBD, 30×150 mm, 55-63% acetonitrile/water gradient buffered with 0.1% formic acid) to give the title compound as a white solid (103 mg). ¹H NMR (400 MHz, DMSO-d₆) δ 12.45 (s, 1H), 8.02 (s, 1H), 7.99-7.92 (m, 1H), 7.20-7.12 (m, 1H), 7.03-6.93 (m, 1H), 6.37-6.28 (m, 2H), 4.18 (q, 2H), 3.89 (t, 4H), 2.33 (p, 2H), 1.25 (t, 3H); LC-MS (method 5): Rₜ=2.94 min; MS (ESIpos): m/z=437 [M+H]⁺

### Example 209

### 6-(azetidin-1-yl)-N-[2-ethoxy-5-(trifluoromethyl)phenyl]sulfonyl-4-fluoro-benzofuran-2-carboxamide

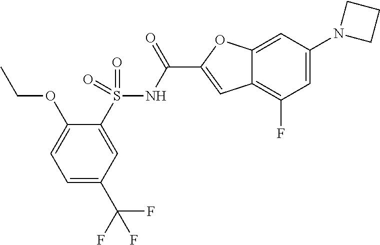 [SMILES: CCOc1ccc(C(F)(F)F)cc1S(=O)(=O)NC(=O)c1cc2c(F)cc(N3CCC3)cc2o1]

To a room temperature stirred solution of 6-(azetidin-1-yl)-4-fluoro-benzofuran-2-carboxylic acid (11.8 mg, 0.05 mmol, 1.00 eq.) in anhydrous tetrahydrofuran (0.25 mL, 0.20 M) was added carbonyldiimidazole (9.73 mg, 0.06 mmol, 1.20 eq.). After stirring for 1 hour, 2-ethoxy-5-(trifluoromethyl)benzenesulfonamide (14.8 mg, 0.05 mmol, 1.10 eq.) and 1,8-diazabicyclo[5.4.0]undec-7-ene (10.0 μL, 0.07 mmol, 1.40 eq.) were sequentially added. The resulting mixture was stirred at room temperature for 16 h and then concentrated down under reduced pressure. The residue was purified by reverse phase preparative column chromatography (Waters XBridge PREP C₁₈, 5 μM OBD, 19×250 mm, 60-68% acetonitrile/water gradient buffered with 0.1% formic acid) to give the title compound as a yellow solid (7.15 mg). ¹H NMR (400 MHz, DMSO-d₆) δ 12.73 (s, 1H), 8.15-8.09 (m, 1H), 8.05-7.94 (m, 2H), 7.46-7.39 (m, 1H), 6.36-6.28 (m, 2H), 4.27 (q, 2H), 3.89 (t, 4H), 2.38-2.28 (m, 2H), 1.31-1.21 (m, 3H); LC-MS (method 5): Rₜ=3.14 min; MS (ESIpos): m/z=487 [M+H]⁺

### Example 210

### 6-(Azetidin-1-yl)-N-(2-ethoxy-5-fluoro-phenyl)sulfonyl-4-fluoro-benzofuran-2-carboxamide

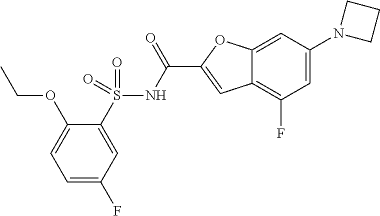 [SMILES: CCOc1ccc(F)cc1S(=O)(=O)NC(=O)c1cc2c(F)cc(N3CCC3)cc2o1]

To a room temperature stirred solution of 6-(azetidin-1-yl)-4-fluoro-benzofuran-2-carboxylic acid (94.1 mg, 0.40 mmol, 1.00 eq.) in anhydrous tetrahydrofuran (2.00 mL, 0.20 M) was added carbonyldiimidazole (77.8 mg, 0.48 mmol, 1.20 eq.). After stirring for 1 hour, 2-ethoxy-5-fluoro-benzenesulfonamide (96.5 mg, 0.44 mmol, 1.10 eq.) and 1,8-diazabicyclo[5.4.0]undec-7-ene (80.0 μL, 0.56 mmol, 1.40 eq.) were sequentially added. The resulting mixture was stirred at room temperature for one hour and then concentrated down under reduced pressure. The residue was purified by reverse phase preparative column chromatography (Waters XBridge PREP C₁₈, 5 μM OBD, 30×150 mm, 55-63% acetonitrile/water gradient buffered with 0.1% formic acid) to give the title compound as a yellow solid (62.2 mg). ¹H NMR (400 MHz, DMSO-d₆) δ 12.57 (s, 1H), 8.03 (s, 1H), 7.67 (dd, 1H), 7.59-7.49 (m, 1H), 7.27 (dd, 1H), 6.37-6.28 (m, 2H), 4.15 (q, 2H), 3.90 (t, 4H), 2.39-2.27 (m, 2H), 1.24 (t, 3H), LC-MS (method 5): Rₜ=2.93 min; MS (ESIpos): m/z=437 [M+H]⁺

### Example 211

### 6-(Azetidin-1-yl)-N-(2-ethoxy-5-phenoxy-phenyl)sulfonyl-4-fluoro-benzofuran-2-carboxamide

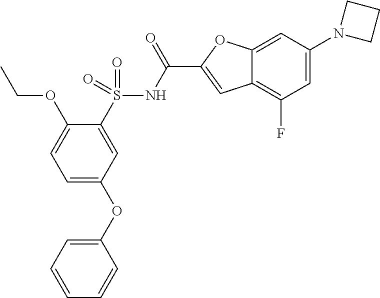 [SMILES: CCOc1ccc(Oc2ccccc2)cc1S(=O)(=O)NC(=O)c1cc2c(F)cc(N3CCC3)cc2o1]

To a room temperature stirred solution of 6-(azetidin-1-yl)-4-fluoro-benzofuran-2-carboxylic acid (94.1 mg, 0.40 mmol, 1.00 eq.) in anhydrous tetrahydrofuran (2.00 mL, 0.20 M) was added carbonyldiimidazole (77.8 mg, 0.48 mmol, 1.20 eq.). After stirring for 1 h, 2-ethoxy-5-phenoxy-benzenesulfonamide (129 mg, 0.44 mmol, 1.10 eq.) and 1,8-diazabicyclo[5.4.0]undec-7-ene (80.0 μL, 0.56 mmol, 1.40 eq.) were sequentially added. The resulting mixture was stirred at room temperature for 16 h and then concentrated down under reduced pressure. The residue was purified by reverse phase preparative column chromatography (Waters XBridge PREP C₁₈, 5 μM OBD, 30×150 mm, 65-73% acetonitrile/water gradient buffered with 0.1% formic acid) to give the title compound as a white solid (152 mg). ¹H NMR (400 MHz, DMSO-d₆) δ 12.52 (s, 1H), 8.04 (s, 1H), 7.49 (d, 1H), 7.46-7.34 (m, 3H), 7.27 (d, Hz, 1H), 7.21-7.12 (m, 1H), 7.08-7.00 (m, 2H), 6.39-6.30 (m, 2H), 4.16 (q, 2H), 3.91 (t, 4H), 2.40-2.28 (m, 2H), 1.26 (t, 3H); LC-MS (method 5): Rₜ=3.32 min; MS (ESIpos): m/z=511 [M+H]⁺

### Example 212

### 6-(Azetidin-1-yl)-N-[2-ethoxy-5-(trifluoromethoxy)phenyl]sulfonyl-4-fluoro-benzofuran-2-carboxamide

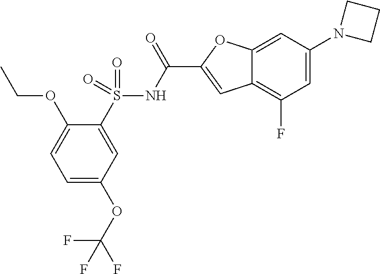 [SMILES: CCOc1ccc(OC(F)(F)F)cc1S(=O)(=O)NC(=O)c1cc2c(F)cc(N3CCC3)cc2o1]

To a room temperature stirred solution of 6-(azetidin-1-yl)-4-fluoro-benzofuran-2-carboxylic acid (96.4 mg, 0.41 mmol, 1.00 eq.) in anhydrous tetrahydrofuran (2.05 mL, 0.20 M) was added carbonyldiimidazole (80.0 mg, 0.49 mmol, 1.20 eq.). After stirring for 1 hour, 2-ethoxy-5-(trifluoromethoxy)benzenesulfonamide (129 mg, 0.45 mmol, 1.10 eq.) and 1,8-diazabicyclo[5.4.0]undec-7-ene (90.0 μL, 0.57 mmol, 1.40 eq.) were sequentially added. The resulting mixture was stirred at room temperature overnight and then concentrated under reduced pressure. The residue was purified by reverse phase preparative column chromatography (Waters XBridge PREP C₁₈, 5 μM OBD, 30×150 mm, 62-70% acetonitrile/water gradient buffered with 0.1% formic acid) to give the title compound as a white solid (93 mg). 1H NMR (400 MHz, DMSO-d₆) δ 12.67-12.62 (m, 1H), 8.02 (s, 1H), 7.83-7.77 (m, 1H), 7.74-7.66 (m, 1H), 7.35 (d, 1H), 6.37-6.29 (m, 2H), 4.21 (q, 2H), 3.90 (t, 4H), 2.39-2.27 (m, 2H), 1.25 (t, 3H); LC-MS (method 5): Rₜ=3.20 min; MS (ESIpos): m/z=503 [M+H]⁺

### Example 213

### 6-(azetidin-1-yl)-N-(2-benzyloxy-5-isopropyl-phenyl)sulfonyl-4-fluoro-benzofuran-2-carboxamide

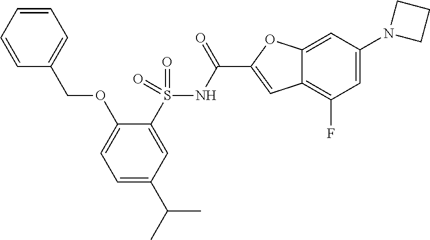 [SMILES: CC(C)c1ccc(OCc2ccccc2)c(S(=O)(=O)NC(=O)c2cc3c(F)cc(N4CCC4)cc3o2)c1]

To a room temperature stirred solution of 6-(azetidin-1-yl)-4-fluoro-benzofuran-2-carboxylic acid (101 mg, 0.43 mmol, 1.00 eq.) in anhydrous tetrahydrofuran (2.15 mL, 0.20 M) was added carbonyldiimidazole (83.7 mg, 0.52 mmol, 1.20 eq.). After stirring for 1 hour, 2-benzyloxy-5-isopropyl-benzenesulfonamide (144 mg, 0.47 mmol, 1.10 eq.) and 1,8-diazabicyclo[5.4.0]undec-7-ene (90.0 μL, 0.60 mmol, 1.40 eq.) were sequentially added. The resulting mixture was stirred at room temperature for 16 h and then concentrated under reduced pressure. The residue was purified by reverse phase preparative column chromatography (Waters XBridge PREP C₁₈, 5 μM OBD, 30×150 mm, 66-74% acetonitrile/water gradient buffered with 0.1% formic acid) to give the title compound as an orange solid (138 mg). 1H NMR (400 MHz, DMSO-d₆) δ 12.53-12.48 (m, 1H), 7.98-7.93 (m, 1H), 7.79-7.74 (m, 1H), 7.51-7.44 (m, 1H), 7.40-7.33 (m, 2H), 7.24-7.08 (m, 4H), 6.36-6.31 (m, 2H), 5.31-5.26 (m, 2H), 3.95-3.86 (m, 4H), 2.98-2.89 (m, 1H), 2.38-2.29 (m, 2H), 1.23-1.16 (m, 6H), LC-MS (method 5): Rₜ=3.45 min; MS (ESIpos): m/z=523 [M+H]⁺

### Example 214

### 6-(azetidin-1-yl)-N-[2-(cyclopropylmethoxy)-5-isopropyl-phenyl]sulfonyl-4-fluoro-benzofuran-2-carboxamide

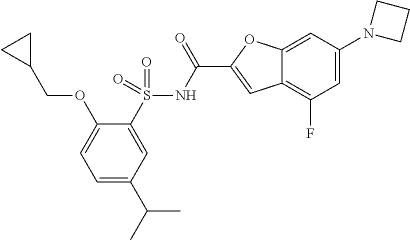 [SMILES: CC(C)c1ccc(OCC2CC2)c(S(=O)(=O)NC(=O)c2cc3c(F)cc(N4CCC4)cc3o2)c1]

To a room temperature stirred solution of 6-(azetidin-1-yl)-4-fluoro-benzofuran-2-carboxylic acid (94.1 mg, 0.40 mmol, 1.00 eq.) in anhydrous tetrahydrofuran (2.00 mL, 0.20 M) was added carbonyldiimidazole (77.8 mg, 0.48 mmol, 1.20 eq.). After stirring for 1 hour, 2-(cyclopropylmethoxy)-5-isopropyl-benzenesulfonamide (119 mg, 0.44 mmol, 1.10 eq.) and 1,8-diazabicyclo[5.4.0]undec-7-ene (80.0 μL, 0.56 mmol, 1.40 eq.) were sequentially added. The resulting mixture was stirred at room temperature over 84 h and then concentrated under reduced pressure. The residue was purified by reverse phase preparative column chromatography (Waters XBridge PREP C₁₈, 5 μM OBD, 30×150 mm, 66-74% acetonitrile/water gradient buffered with 0.1% formic acid) to give the title compound as a white solid (123 mg). ¹H NMR (400 MHz, DMSO-d₆) δ 12.32 (s, 1H), 8.03 (s, 1H), 7.73 (s, 1H), 7.54-7.47 (m, 1H), 7.14 (d, 1H), 6.36-6.28 (m, 2H), 3.97-3.85 (m, 6H), 3.00-2.91 (m, 1H), 2.37-2.29 (m, 2H), 1.21 (d, 7H), 0.38-0.31 (m, 2H), 0.19-0.13 (m, 2H), LC-MS (method 5): Rₜ=3.38 min; MS (ESIpos): m/z=487 [M+H]⁺

### Example 215

### 6-(azetidin-1-yl)-4-fluoro-N-[5-isopropyl-2-(2,2,2-trifluoroethoxy)phenyl]sulfonyl-benzofuran-2-carboxamide

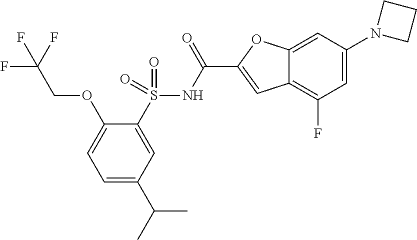 [SMILES: CC(C)c1ccc(OCC(F)(F)F)c(S(=O)(=O)NC(=O)c2cc3c(F)cc(N4CCC4)cc3o2)c1]

To a room temperature stirred solution of 6-(azetidin-1-yl)-4-fluoro-benzofuran-2-carboxylic acid (70.6 mg, 0.30 mmol, 1.00 eq.) in anhydrous tetrahydrofuran (1.50 mL, 0.20 M) was added carbonyldiimidazole (58.4 mg, 0.36 mmol, 1.20 eq.). After stirring for 1 hour, 5-isopropyl-2-(2,2,2-trifluoroethoxy)benzenesulfonamide (98.1 mg, 0.33 mmol, 1.10 eq.) and 1,8-diazabicyclo[5.4.0]undec-7-ene (60.0 μL, 0.42 mmol, 1.40 eq.) were sequentially added. The resulting mixture was stirred at room temperature for 18 h and then concentrated under reduced pressure. The residue was purified by reverse phase preparative column chromatography (Waters XBridge PREP C₁₈, 5 μM OBD, 30×150 mm, 64-72% acetonitrile/water gradient buffered with 0.1% formic acid) to give the title compound as a yellow solid (102.5 mg). ¹H NMR (400 MHz, DMSO-d₆) δ 12.47 (s, 1H), 7.96 (s, 1H), 7.79 (d, 1H), 7.63-7.55 (m, 1H), 7.28 (d, 1H), 6.36-6.28 (m, 2H), 4.93-4.82 (m, 2H), 3.89 (t, 4H), 3.05-2.93 (m, 1H), 2.39-2.27 (m, 1H), 2.07 (s, 1H), 1.22 (d, 6H), LC-MS (method 5): Rₜ=3.28 min; MS (ESIpos): m/z=515 [M+H]⁺

### Example 216

### N-(benzenesulfonyl)-6-(2,2-dimethylazetidin-1-yl)-4-fluoro-benzofuran-2-carboxamide

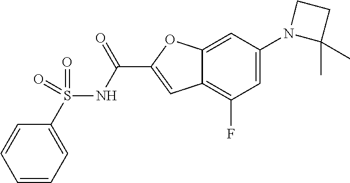 [SMILES: CC1(C)CCN1c1cc(F)c2cc(C(=O)NS(=O)(=O)c3ccccc3)oc2c1]

To a room temperature stirred solution of 6-(2,2-dimethylazetidin-1-yl)-4-fluoro-benzofuran-2-carboxylic acid (105 mg, 0.40 mmol, 1.00 eq.) in anhydrous tetrahydrofuran (2.00 mL, 0.20 M) was added carbonyldiimidazole (77.8 mg, 0.48 mmol, 1.20 eq.). After stirring for 1 hour, benzenesulfonamide (69.2 mg, 0.44 mmol, 1.10 eq.) and 1,8-diazabicyclo[5.4.0]undec-7-ene (83.8 μL, 0.56 mmol, 1.40 eq.) were sequentially added. The resulting mixture was stirred at room temperature for a further 1 h and then concentrated under reduced pressure. The residue was purified by reverse phase preparative column chromatography (Waters XBridge PREP C₁₈, 5 μM OBD, 30×150 mm, 57-65% acetonitrile/water gradient buffered with 0.1% formic acid) to give the title compound as a yellow solid (112 mg). ¹H NMR (400 MHz, DMSO-d₆) δ 12.59 (s, 1H), 8.03-7.96 (m, 2H), 7.89-7.84 (m, 1H), 7.77-7.69 (m, 1H), 7.69-7.60 (m, 2H), 6.36 (s, 1H), 6.31 (dd, 1H), 3.70 (t, 2H), 2.10 (t, 2H), 1.45 (s, 6H), LC-MS (method 5): Rₜ=3.04 min; MS (ESIneg): m/z=401 [M−H]⁻

### Example 217

### 6-(2,2-Dimethylazetidin-1-yl)-4-fluoro-N-(2-phenylphenyl)sulfonyl-benzofuran-2-carboxamide

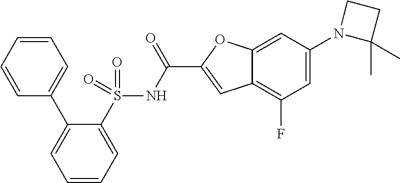 [SMILES: CC1(C)CCN1c1cc(F)c2cc(C(=O)NS(=O)(=O)c3ccccc3-c3ccccc3)oc2c1]

To a room temperature stirred solution of 6-(2,2-dimethylazetidin-1-yl)-4-fluoro-benzofuran-2-carboxylic acid (105 mg, 0.40 mmol, 1.00 eq.) in anhydrous tetrahydrofuran (2.00 mL, 0.20 M) was added carbonyldiimidazole (77.8 mg, 0.48 mmol, 1.20 eq.). After stirring for 1 hour, 2-phenylbenzenesulfonamide (103 mg, 0.44 mmol, 1.10 eq.) and 1,8-diazabicyclo[5.4.0]undec-7-ene (83.8 μL, 0.56 mmol, 1.40 eq.) were sequentially added. The resulting mixture was stirred at room temperature for a further 1 hour and then concentrated under reduced pressure. The residue was purified by reverse phase preparative column chromatography (Waters XBridge PREP C₁₈, 5 μM OBD, 30×150 mm, 66-74% acetonitrile/water gradient buffered with 0.1% formic acid) to give the title compound as a yellow solid (153 mg). ¹H NMR (400 MHz, DMSO-d₆) δ 12.04 (s, 1H), 8.16 (dd, 1H), 7.73 (td, 1H), 7.66 (td, 1H), 7.58 (s, 1H), 7.42-7.22 (m, 7H), 6.38-6.27 (m, 2H), 3.71 (t, 2H), 2.11 (t, 2H), 1.46 (s, 6H), LC-MS (method 5): Rₜ=3.42 min; MS (ESIneg): m/z=478 [M−H]⁻

### Example 218

### 6-(2,2-Dimethylazetidin-1-yl)-4-fluoro-N-[(2-methyl-8-quinolyl)sulfonyl]benzofuran-2-carboxamide

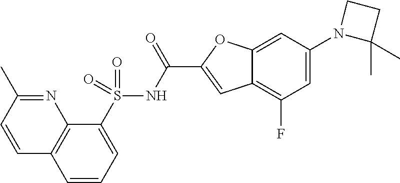 [SMILES: Cc1ccc2cccc(S(=O)(=O)NC(=O)c3cc4c(F)cc(N5CCC5(C)C)cc4o3)c2n1]

To a room temperature stirred solution of 6-(2,2-dimethylazetidin-1-yl)-4-fluoro-benzofuran-2-carboxylic acid (105 mg, 0.40 mmol, 1.00 eq.) in anhydrous tetrahydrofuran (2.00 mL, 0.20 M) was added carbonyldiimidazole (77.8 mg, 0.48 mmol, 1.20 eq.). After stirring for 1 hour, 2-methylquinoline-8-sulfonamide (98.8 mg, 0.44 mmol, 1.10 eq.) and 1,8-diazabicyclo[5.4.0]undec-7-ene (83.8 μL, 0.56 mmol, 1.40 eq.) were sequentially added. The resulting mixture was stirred at room temperature for a further 1 hour and then concentrated under reduced pressure. The residue was purified by reverse phase preparative column chromatography (Waters XBridge PREP C₁₈, 5 μM OBD, 30×150 mm, 56-64% acetonitrile/water gradient buffered with 0.1% formic acid) to give the title compound as a yellow solid (139 mg). ¹H NMR (400 MHz, DMSO-d₆) δ 12.44 (s, 1H), 8.46 (dd, 1H), 8.39 (d, 1H), 8.29 (dd, 1H), 8.13 (s, 1H), 7.75 (t, 1H), 7.52 (d, 1H), 6.30 (d, 2H), 3.67 (t, 2H), 2.69 (s, 3H), 2.12-2.04 (m, 2H), 1.43 (s, 6H), LC-MS (method 5): Rₜ=3.00 min; MS (ESIpos): m/z=468 [M+H]⁺

### Example 219

### 6-(2,2-Dimethylazetidin-1-yl)-N-(2-ethoxyphenyl)sulfonyl-4-fluoro-benzofuran-2-carboxamide

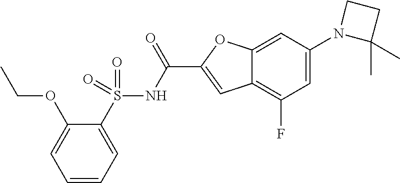 [SMILES: CCOc1ccccc1S(=O)(=O)NC(=O)c1cc2c(F)cc(N3CCC3(C)C)cc2o1]

To a room temperature stirred solution of 6-(2,2-dimethylazetidin-1-yl)-4-fluoro-benzofuran-2-carboxylic acid (105 mg, 0.40 mmol, 1.00 eq.) in anhydrous tetrahydrofuran (2.00 mL, 0.20 M) was added carbonyldiimidazole (77.8 mg, 0.48 mmol, 1.20 eq.). After stirring for 1 hour, 2-ethoxybenzenesulfonamide (88.6 mg, 0.44 mmol, 1.10 eq.) and 1,8-diazabicyclo[5.4.0]undec-7-ene (83.8 μL, 0.56 mmol, 1.40 eq.) were sequentially added. The resulting mixture was stirred at room temperature for a further 1 hour and then concentrated under reduced pressure. The residue was purified by reverse phase preparative column chromatography (Waters XBridge PREP C₁₈, 5 μM OBD, 30×150 mm, 59-67% acetonitrile/water gradient buffered with 0.1% formic acid) to give the title compound as a yellow solid (139 mg). ¹H NMR (400 MHz, DMSO-d₆) δ 12.38 (s, 1H), 8.04 (s, 1H), 7.90 (dd, 1H), 7.64 (ddd, 1H), 7.21 (d, 1H), 7.17-7.08 (m, 1H), 6.39-6.27 (m, 2H), 4.16 (q, 2H), 3.70 (t, 2H), 2.14-2.05 (m, 2H), 1.45 (s, 6H), 1.25 (t, 3H); LC-MS (method 5): Rₜ=3.13 min; MS (ESIpos): m/z=447 [M+H]⁺

### Example 220

### N-(Benzenesulfonyl)-4-fluoro-6-(2-methylazetidin-1-yl)benzofuran-2-carboxamide (rac)

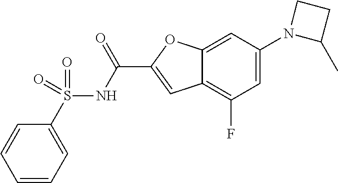 [SMILES: CC1CCN1c1cc(F)c2cc(C(=O)NS(=O)(=O)c3ccccc3)oc2c1]

To a room temperature stirred solution of 4-fluoro-6-(2-methylazetidin-1-yl)benzofuran-2-carboxylic acid (99.7 mg, 0.40 mmol, 1.00 eq.) in anhydrous tetrahydrofuran (2.00 mL, 0.20 M) was added carbonyldiimidazole (77.8 mg, 0.48 mmol, 1.20 eq.). After stirring for 1 h, benzenesulfonamide (69.2 mg, 0.44 mmol, 1.10 eq.) and 1,8-diazabicyclo[5.4.0]undec-7-ene (83.8 μL, 0.56 mmol, 1.40 eq.) were sequentially added. The resulting mixture was stirred at room temperature for a further 16 h and then concentrated under reduced pressure. The residue was purified by reverse phase preparative column chromatography (Waters XBridge PREP C₁₈, 5 μM OBD, 30×150 mm, 54-62% acetonitrile/water gradient buffered with 0.1% formic acid) to give the title compound as a yellow solid (103 mg). ¹H NMR (400 MHz, DMSO-d₆) δ 12.62 (s, 1H), 8.04-7.96 (m, 2H), 7.88 (d, 1H), 7.83-7.68 (m, 1H), 7.65 (ddHz, 2H), 6.42-6.30 (m, 2H), 4.22 (dt, 1H), 3.94 (td, 1H), 3.63 (dt, 1H), 2.43 (dtd, 4.4 Hz, 1H), 1.98 (ddt, 1H), 1.41 (d, 3H), LC-MS (method 5): Rₜ=2.92 min; MS (ESIpos): m/z=389 [M+H]⁺

### Example 221

### 4-Fluoro-6-(2-methylazetidin-1-yl)-N-[(2-methyl-8-quinolyl)sulfonyl]benzofuran-2-carboxamide (rac)

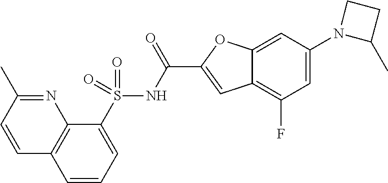 [SMILES: Cc1ccc2cccc(S(=O)(=O)NC(=O)c3cc4c(F)cc(N5CCC5C)cc4o3)c2n1]

To a room temperature stirred solution of 4-fluoro-6-(2-methylazetidin-1-yl)benzofuran-2-carboxylic acid (99.7 mg, 0.40 mmol, 1.00 eq.) in anhydrous tetrahydrofuran (2.00 mL, 0.20 M) was added carbonyldiimidazole (77.8 mg, 0.48 mmol, 1.20 eq.). After stirring for 1 h, 2-methylquinoline-8-sulfonamide (97.8 mg, 0.44 mmol, 1.10 eq.) and 1,8-diazabicyclo[5.4.0]undec-7-ene (83.8 μL, 0.56 mmol, 1.40 eq.) were sequentially added. The resulting mixture was stirred at room temperature for a further 16 h and then concentrated under reduced pressure. The residue was purified by reverse phase preparative column chromatography (Waters XBridge PREP C₁₈, 5 μM OBD, 30×150 mm, 52-60% acetonitrile/water gradient buffered with 0.1% formic acid) to give the title compound as a yellow solid (128 mg). ¹H NMR (400 MHz, DMSO-d₆) δ 12.49 (s, 1H), 8.45 (dd, 1H), 8.39 (d, 1H), 8.29 (dd, 1H), 8.13 (s, 1H), 7.74 (t, 1H), 7.52 (d, 1H), 6.38-6.29 (m, 2H), 4.18 (dt, 1H), 3.91 (td, 1H), 3.65-3.55 (m, 1H), 2.68 (s, 3H), 2.41 (dtd, 1H), 1.96 (ddt, 1H), 1.38 (d, 3H); LC-MS (method 5): Rₜ=2.86 min; MS (ESIpos): m/z=454 [M+H]⁺

### Example 222

### 4-Fluoro-6-(2-methylazetidin-1-yl)-N-(2-phenylphenyl)sulfonyl-benzofuran-2-carboxamide (rac)

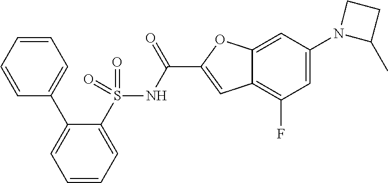 [SMILES: CC1CCN1c1cc(F)c2cc(C(=O)NS(=O)(=O)c3ccccc3-c3ccccc3)oc2c1]

To a room temperature stirred solution of 4-fluoro-6-(2-methylazetidin-1-yl)benzofuran-2-carboxylic acid (99.7 mg, 0.40 mmol, 1.00 eq.) in anhydrous tetrahydrofuran (2.00 mL, 0.20 M) was added carbonyldiimidazole (77.8 mg, 0.48 mmol, 1.20 eq.). After stirring for 1 h, 2-phenylbenzenesulfonamide (103 mg, 0.44 mmol, 1.10 eq.) and 1,8-diazabicyclo[5.4.0]undec-7-ene (83.8 μL, 0.56 mmol, 1.40 eq.) were sequentially added. The resulting mixture was stirred at room temperature for a further 16 h and then concentrated under reduced pressure. The residue was purified by reverse phase preparative column chromatography (Waters XBridge PREP C₁₈, 5 μM OBD, 30×150 mm, 64-72% acetonitrile/water gradient buffered with 0.1% formic acid) to give the title compound as a yellow solid (152 mg). ¹H NMR (400 MHz, DMSO-d₆) δ 12.06 (s, 1H), 8.16 (dd, 1H), 7.73 (td, 1H), 7.66 (td, 1H), 7.60 (s, 1H), 7.40-7.34 (m, 1H), 7.34-7.28 (m, 3H), 7.28-7.22 (m, 2H), 6.39 (t, 1H), 6.35 (dd, 1H), 4.22 (dt, 1H), 3.94 (td, 1H), 3.70-3.58 (m, 1H), 2.44 (dtd, 1H), 1.99 (ddt, 1H), 1.41 (d, 3H), LC-MS (method 5): Rₜ=3.32 min; MS (ESIpos): m/z=465 [M+H]⁺

### Example 223

### N-(2-Ethoxyphenyl)sulfonyl-4-fluoro-6-(2-methylazetidin-1-yl)benzofuran-2-carboxamide (rac)

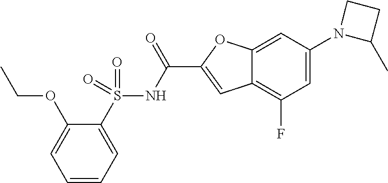 [SMILES: CCOc1ccccc1S(=O)(=O)NC(=O)c1cc2c(F)cc(N3CCC3C)cc2o1]

To a room temperature stirred solution of 4-fluoro-6-(2-methylazetidin-1-yl)benzofuran-2-carboxylic acid (99.7 mg, 0.40 mmol, 1.00 eq.) in anhydrous tetrahydrofuran (2.00 mL, 0.20 M) was added carbonyldiimidazole (77.8 mg, 0.48 mmol, 1.20 eq.). After stirring for 1 hour, 2-ethoxybenzenesulfonamide (88.6 mg, 0.44 mmol, 1.10 eq.) and 1,8-diazabicyclo[5.4.0]undec-7-ene (83.8 μL, 0.56 mmol, 1.40 eq.) were sequentially added. The resulting mixture was stirred at room temperature for a further 18 h and then concentrated under reduced pressure. The residue was purified by reverse phase preparative column chromatography (Waters XBridge PREP C₁₈, 5 μM OBD, 30×150 mm, 50-58% acetonitrile/water gradient buffered with 0.1% formic acid) to give the title compound as a light yellow solid (146 mg). ¹H NMR (400 MHz, DMSO-d₆) δ 12.41 (s, 1H), 8.05 (s, 1H), 7.90 (dd, 1H), 7.64 (ddd, 1H), 7.21 (d, 1H), 7.17-7.08 (m, 1H), 6.41-6.39 (m, 1H), 6.35 (dd, 1H), 4.27-4.10 (m, 3H), 3.93 (td, 1H), 3.63 (dt, 1H), 2.43 (dtd, 1H), 1.98 (ddt, 1H), 1.41 (d, 3H), 1.24 (t, 3H); LC-MS (method 5): Rₜ=3.03 min; MS (ESIpos): m/z=433 [M+H]⁺

### Example 224

### 6-(Azetidin-1-yl)-N-[(5-(tert-butyl)-2-cyclopropoxyphenyl)sulfonyl]-4-fluorobenzofuran-2-carboxamide

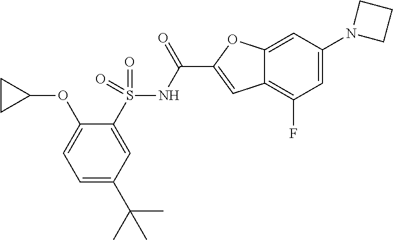 [SMILES: CC(C)(C)c1ccc(OC2CC2)c(S(=O)(=O)NC(=O)c2cc3c(F)cc(N4CCC4)cc3o2)c1]

To a room temperature stirred solution of 6-(azetidin-1-yl)-4-fluoro-benzofuran-2-carboxylic acid (63.5 mg, 0.27 mmol, 1.00 eq.) in anhydrous tetrahydrofuran (1.35 mL, 0.20 M) was added carbonyldiimidazole (52.5 mg, 0.32 mmol, 1.20 eq.). After stirring for 1 h, 5-tert-butyl-2-(cyclopropoxy)benzenesulfonamide (80.0 mg, 0.30 mmol, 1.10 eq.) and 1,8-diazabicyclo[5.4.0]undec-7-ene (60.0 μL, 0.38 mmol, 1.40 eq.) were sequentially added. The resulting mixture was stirred at room temperature for 15 h and then concentrated under reduced pressure. The residue was purified by reverse phase preparative column chromatography (Waters XBridge PREP C₁₈, 5 μM OBD, 30×150 mm, 63-71% acetonitrile/water gradient buffered with 0.1% formic acid) to give the title compound as a yellow solid (91 mg). ¹H NMR (400 MHz, DMSO-d₆) δ 12.32 (s, 1H), 7.99 (s, 1H), 7.84 (d, 1H), 7.75-7.68 (m, 1H), 7.39 (d, 1H), 6.37-6.29 (m, 2H), 4.08-3.98 (m, 1H), 3.90 (t, 4H), 2.33 (p, 2H), 1.31 (s, 9H), 0.76-0.66 (m, 2H), 0.57-0.48 (m, 2H); LC-MS (method 5): Rₜ=3.26 min; MS (ESIpos): m/z=487 [M+H]⁺

### Example 225

### 6-(Azetidin-1-yl)-N-[(2-(benzyloxy)-5-(tert-butyl)phenyl]sulfonyl)-4-fluorobenzofuran-2-carboxamide

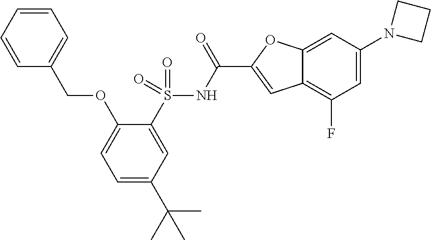 [SMILES: CC(C)(C)c1ccc(OCc2ccccc2)c(S(=O)(=O)NC(=O)c2cc3c(F)cc(N4CCC4)cc3o2)c1]

To a room temperature stirred solution of 6-(azetidin-1-yl)-4-fluoro-benzofuran-2-carboxylic acid (106 mg, 0.45 mmol, 1.00 eq.) in anhydrous tetrahydrofuran (2.25 mL, 0.20 M) was added carbonyldiimidazole (87.6 mg, 0.54 mmol, 1.20 eq.). After stirring for 1 h, 2-benzyloxy-5-tert-butyl-benzenesulfonamide (158 mg, 0.49 mmol, 1.10 eq.) and 1,8-diazabicyclo[5.4.0]undec-7-ene (90.0 μL, 0.63 mmol, 1.40 eq.) were sequentially added. The resulting mixture was stirred at room temperature for 15 h and then concentrated under reduced pressure. The residue was purified by reverse phase preparative column chromatography (Waters XBridge PREP C₁₈, 5 μM OBD, 30×150 mm, 67-75% acetonitrile/water gradient buffered with 0.1% formic acid) to give the title compound as a yellow solid (97.3 mg). ¹H NMR (400 MHz, DMSO-d₆) δ 12.57 (s, 1H), 7.97 (s, 1H), 7.88 (d, 1H), 7.66-7.58 (m, 1H), 7.37 (d, 2H), 7.24-7.08 (m, 4H), 6.39-6.31 (m, 2H), 5.29 (s, 2H), 3.91 (t, 4H), 2.33 (p, 2H), 1.28 (s, 9H), LC-MS (method 5): Rₜ=3.44 min; MS (ESIpos): m/z=537 [M+H]⁺

### Example 226

### 6-(Azetidin-1-yl)-N-[(5-(tert-butyl)-2-(cyclopropylmethoxy)phenyl)sulfonyl]-4-fluorobenzofuran-2-carboxamide

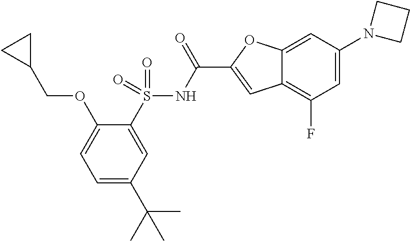 [SMILES: CC(C)(C)c1ccc(OCC2CC2)c(S(=O)(=O)NC(=O)c2cc3c(F)cc(N4CCC4)cc3o2)c1]

To a room temperature stirred solution of 6-(azetidin-1-yl)-4-fluoro-benzofuran-2-carboxylic acid (106 mg, 0.45 mmol, 1.00 eq.) in anhydrous tetrahydrofuran (2.25 mL, 0.20 M) was added carbonyldiimidazole (87.6 mg, 0.54 mmol, 1.20 eq.). After stirring for 1 hour, 5-tert-butyl-2-(cyclopropylmethoxy)benzenesulfonamide (140 mg, 0.49 mmol, 1.10 eq.) and 1,8-diazabicyclo[5.4.0]undec-7-ene (90.0 μL, 0.63 mmol, 1.40 eq.) were sequentially added. The resulting mixture was stirred at room temperature for 15 h and then concentrated under reduced pressure. The residue was purified by reverse phase preparative column chromatography (Waters XBridge PREP C₁₈, 5 μM OBD, 30×150 mm, 66-74% acetonitrile/water gradient buffered with 0.1% formic acid) to give the title compound as a yellow solid (171 mg). ¹H NMR (400 MHz, DMSO-d₆) δ 12.38 (s, 1H), 8.04 (s, 1H), 7.86 (d, 1H), 7.69-7.62 (m, 1H), 7.15 (d, 1H), 6.37-6.29 (m, 2H), 3.97-3.85 (m, 6H), 2.32 (p, 2H), 1.30 (s, 9H), 1.25-1.11 (m, 1H), 0.38-0.27 (m, 2H), 0.23-0.12 (m, 2H), LC-MS (method 5): Rₜ=3.38 min; MS (ESIpos): m/z=501 [M+H]⁺

### Example 227

### 6-(Azetidin-1-yl)-N-[(5-(tert-butyl)-2-(2,2,2-trifluoroethoxy)phenyl)sulfonyl]-4-fluorobenzofuran-2-carboxamide

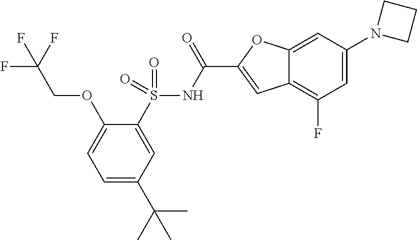 [SMILES: CC(C)(C)c1ccc(OCC(F)(F)F)c(S(=O)(=O)NC(=O)c2cc3c(F)cc(N4CCC4)cc3o2)c1]

To a room temperature stirred solution of 6-(azetidin-1-yl)-4-fluoro-benzofuran-2-carboxylic acid (101 mg, 0.43 mmol, 1.00 eq.) in anhydrous tetrahydrofuran (2.15 mL, 0.20 M) was added carbonyldiimidazole (83.7 mg, 0.52 mmol, 1.20 eq.). After stirring for 1 hour, 5-tert-butyl-2-(2,2,2-trifluoroethoxy)benzenesulfonamide (147 mg, 0.47 mmol, 1.10 eq.) and 1,8-diazabicyclo[5.4.0]undec-7-ene (90.0 μL, 0.60 mmol, 1.40 eq.) were sequentially added. The resulting mixture was stirred at room temperature for 15 h and then concentrated down under reduced pressure. The residue was purified by reverse phase preparative column chromatography (Waters XBridge PREP C₁₈, 5 μM OBD, 30×150 mm, 64-72% acetonitrile/water gradient buffered with 0.1% formic acid) to give the title compound as a yellow solid (179 mg). ¹H NMR (400 MHz, DMSO-d₆) δ 12.53 (s, 1H), 7.97 (s, 1H), 7.91 (d, 1H), 7.74 (dd, 1H), 7.28 (d, 1H), 6.37-6.29 (m, 2H), 4.90 (q, 2H), 3.89 (t, 4H), 2.32 (p, 2H), 1.31 (s, 9H); LC-MS (method 5): Rₜ=3.27 min; MS (ESIpos): m/z=529 [M+H]⁺

### Example 228

### 6-(Azetidin-1-yl)-N-[(5-(tert-butyl)-2-cyclobutoxyphenyl)sulfonyl]-4-fluorobenzofuran-2-carboxamide

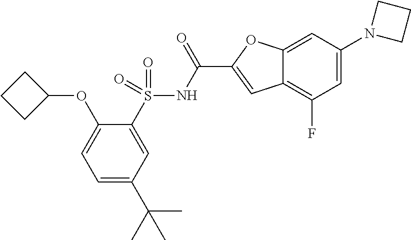 [SMILES: CC(C)(C)c1ccc(OC2CCC2)c(S(=O)(=O)NC(=O)c2cc3c(F)cc(N4CCC4)cc3o2)c1]

To a room temperature stirred solution of 6-(azetidin-1-yl)-4-fluoro-benzofuran-2-carboxylic acid (106 mg, 0.45 mmol, 1.00 eq.) in anhydrous tetrahydrofuran (2.25 mL, 0.20 M) was added carbonyldiimidazole (87.6 mg, 0.54 mmol, 1.20 eq.). After stirring for 1 h, 5-tert-butyl-2-(cyclobutoxy)benzenesulfonamide (140 mg, 0.49 mmol, 1.10 eq.) and 1,8-diazabicyclo[5.4.0]undec-7-ene (90.0 μL, 0.63 mmol, 1.40 eq.) were sequentially added. The resulting mixture was stirred at room temperature for 15 h and then concentrated under reduced pressure. The residue was purified by reverse phase preparative column chromatography (Waters XBridge PREP C₁₈, 5 μM OBD, 30×150 mm, 67-75% acetonitrile/water gradient buffered with 0.1% formic acid) to give the title compound as a yellow solid (151 mg). ¹H NMR (400 MHz, DMSO-d₆) δ 12.39 (s, 1H), 8.08 (s, 1H), 7.84 (d, 1H), 7.67-7.60 (m, 1H), 6.95 (d, 1H), 6.37-6.29 (m, 2H), 4.88-4.76 (m, 1H), 3.89 (t, 4H), 2.39-2.24 (m, 4H), 2.04-1.89 (m, 2H), 1.57-1.41 (m, 2H), 1.29 (s, 9H); LC-MS (method 5): Rₜ=3.40 min; MS (ESIpos): m/z=501 [M+H]⁺

### Example 229

### 6-(Azetidin-1-yl)-N-[(5-(tert-butyl)-2-isopropoxyphenyl)sulfonyl]-4-fluorobenzofuran-2-carboxamide

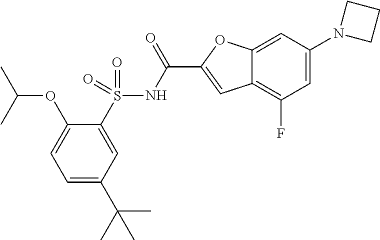 [SMILES: CC(C)Oc1ccc(C(C)(C)C)cc1S(=O)(=O)NC(=O)c1cc2c(F)cc(N3CCC3)cc2o1]

To a room temperature stirred solution of 6-(azetidin-1-yl)-4-fluoro-benzofuran-2-carboxylic acid (106 mg, 0.45 mmol, 1.00 eq.) in anhydrous tetrahydrofuran (2.25 mL, 0.20 M) was added carbonyldiimidazole (87.6 mg, 0.54 mmol, 1.20 eq.). After stirring for 1 hour, 5-tert-butyl-2-isopropoxy-benzenesulfonamide (134 mg, 0.49 mmol, 1.10 eq.) and 1,8-diazabicyclo[5.4.0]undec-7-ene (90.0 μL, 0.63 mmol, 1.40 eq.) were sequentially added. The resulting mixture was stirred at room temperature for 15 h and then concentrated under reduced pressure. The residue was purified by reverse phase preparative column chromatography (Waters XBridge PREP C₁₈, 5 μM OBD, 30×150 mm, 65-73% acetonitrile/water gradient buffered with 0.1% formic acid) to give the title compound as a yellow solid (172 mg). ¹H NMR (400 MHz, DMSO-d₆) δ 12.29 (s, 1H), 8.05 (s, 1H), 7.84 (d, 1H), 7.69-7.61 (m, 1H), 7.16 (d, 1H), 6.37-6.29 (m, 2H), 4.76 (p, 1H), 3.89 (t, 4H), 2.39-2.26 (m, 2H), 1.30 (s, 9H), 1.17 (d, 6H), LC-MS (method 5): Rₜ=3.35 min; MS (ESIpos): m/z=489 [M+H]⁺

### Example 230

### 6-(Azetidin-1-yl)-N-[(2-cyclobutoxy-5-isopropylphenyl)sulfonyl]-4-fluorobenzofuran-2-carboxamide

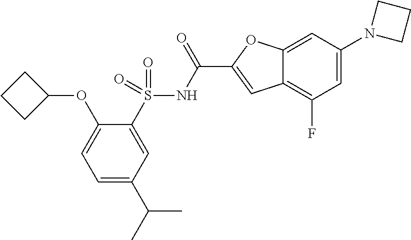 [SMILES: CC(C)c1ccc(OC2CCC2)c(S(=O)(=O)NC(=O)c2cc3c(F)cc(N4CCC4)cc3o2)c1]

To a room temperature stirred solution of 6-(azetidin-1-yl)-4-fluoro-benzofuran-2-carboxylic acid (94.1 mg, 0.40 mmol, 1.00 eq.) in anhydrous tetrahydrofuran (2.00 mL, 0.20 M) was added carbonyldiimidazole (77.8 mg, 0.48 mmol, 1.20 eq.). After stirring for 1 h, 2-(cyclobutoxy)-5-isopropyl-benzenesulfonamide (119 mg, 0.44 mmol, 1.10 eq.) and 1,8-diazabicyclo[5.4.0]undec-7-ene (80.0 μL, 0.56 mmol, 1.40 eq.) were sequentially added. The resulting mixture was stirred at room temperature for one hour and then concentrated under reduced pressure. The residue was purified by reverse phase preparative column chromatography (Waters XBridge PREP C₁₈, 5 μM OBD, 30×150 mm, 67-75% acetonitrile/water gradient buffered with 0.1% formic acid) to give the title compound as yellow solid (135 mg). ¹H NMR (400 MHz, DMSO-d₆) δ 12.40 (s, 1H), 8.09 (s, 1H), 7.73 (d, 1H), 7.53-7.45 (m, 1H), 6.99-6.92 (m, 1H), 6.38-6.30 (m, 2H), 4.88-4.76 (m, 1H), 3.90 (t, 4H), 3.03-2.89 (m, 1H), 2.40-2.24 (m, 4H), 2.05-1.90 (m, 2H), 1.57-1.41 (m, 2H), 1.21 (d, 6H); LC-MS (method 5): Rₜ=3.33 min; MS (ESIpos): m/z=487 [M+H]⁺

### Example 231

### 6-(Azetidin-1-yl)-N-((2-((2,2-difluorocyclopropyl)methoxy)-5-isopropylphenyl)sulfonyl)-4-fluorobenzofuran-2-carboxamide (rac)

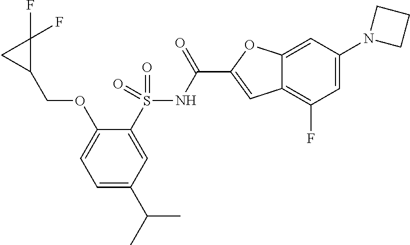 [SMILES: CC(C)c1ccc(OCC2CC2(F)F)c(S(=O)(=O)NC(=O)c2cc3c(F)cc(N4CCC4)cc3o2)c1]

To a room temperature stirred solution of 6-(azetidin-1-yl)-4-fluoro-benzofuran-2-carboxylic acid (80.0 mg, 0.34 mmol, 1.00 eq.) in anhydrous tetrahydrofuran (1.70 mL, 0.20 M) was added carbonyldiimidazole (66.2 mg, 0.41 mmol, 1.20 eq.). After stirring for 1 hour, 2-[(2,2-difluorocyclopropyl)methoxy]-5-isopropyl-benzenesulfonamide (114 mg, 0.37 mmol, 1.10 eq.) and 1,8-diazabicyclo[5.4.0]undec-7-ene (70.0 μL, 0.48 mmol, 1.40 eq.) were sequentially added. The resulting mixture was stirred at room temperature for one hour and then concentrated under reduced pressure. The residue was purified by reverse phase preparative column chromatography (Waters XBridge PREP C₁₈, 5 μM OBD, 30×150 mm, 64-72% acetonitrile/water gradient buffered with 0.1% formic acid) to give the title compound as a yellow solid (127 mg). 1H NMR (400 MHz, DMSO-d₆) δ 12.39 (s, 1H), 8.00 (s, 1H), 7.79-7.73 (m, 1H), 7.58-7.50 (m, 1H), 7.20 (d, 1H), 6.35-6.27 (m, 2H), 4.22-4.15 (m, 2H), 3.89 (t, 4H), 3.03-2.89 (m, 1H), 2.37-2.28 (m, 2H), 2.28-2.13 (m, 1H), 1.61-1.47 (m, 1H), 1.41-1.28 (m, 1H), 1.22 (d, 6H); LC-MS (method 5): Rₜ=3.31 min; MS (ESIpos): m/z=523 [M+H]⁺

### Example 232

### 6-(Azetidin-1-yl)-N-((2-ethoxy-4,5-difluorophenyl)sulfonyl)-4-fluorobenzofuran-2-carboxamide

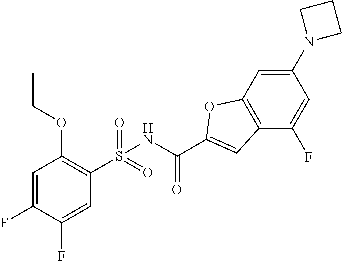 [SMILES: CCOc1cc(F)c(F)cc1S(=O)(=O)NC(=O)c1cc2c(F)cc(N3CCC3)cc2o1]

To a room temperature stirred solution of 6-(azetidin-1-yl)-4-fluoro-benzofuran-2-carboxylic acid (94.1 mg, 0.40 mmol, 1.00 eq.) in anhydrous tetrahydrofuran (2.00 mL, 0.20 M) was added carbonyldiimidazole (77.8 mg, 0.48 mmol, 1.20 eq.). After stirring for 1 hour, 2-ethoxy-4,5-difluoro-benzenesulfonamide (104 mg, 0.44 mmol, 1.10 eq.) and 1,8-diazabicyclo[5.4.0]undec-7-ene (80.0 μL, 0.56 mmol, 1.40 eq.) were sequentially added. The resulting mixture was stirred at room temperature for 15 h and then concentrated under reduced pressure. The residue was purified by reverse phase preparative column chromatography (Waters XBridge PREP C₁₈, 5 μM OBD, 30×150 mm, 54-62% acetonitrile/water gradient buffered with 0.1% formic acid) to give the title compound as a yellow solid (121 mg). ¹H NMR (400 MHz, DMSO-d₆) δ 8.01 (s, 1H), 7.92 (t, 1H), 7.46 (dd, 1H), 6.37-6.28 (m, 2H), 4.17 (q, 2H), 3.89 (t, 4H), 2.33 (p, 2H), 1.24 (t, 3H), LC-MS (method 5): Rₜ=2.92 min; MS (ESIpos): m/z=455 [M+H]⁺

### Example 233

### 6-(Azetidin-1-yl)-4-fluoro-N-[(2-methoxy-5-(methoxymethyl)phenyl)sulfonyl]benzofuran-2-carboxamide

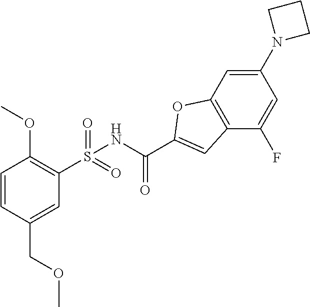 [SMILES: COCc1ccc(OC)c(S(=O)(=O)NC(=O)c2cc3c(F)cc(N4CCC4)cc3o2)c1]

To a room temperature stirred solution of 6-(azetidin-1-yl)-4-fluoro-benzofuran-2-carboxylic acid (9.41 mg, 0.04 mmol, 1.00 eq.) in anhydrous tetrahydrofuran (0.20 mL, 0.20 M) was added carbonyldiimidazole (7.78 mg, 0.05 mmol, 1.20 eq.). After stirring for 1 hour, 2-methoxy-5-(methoxymethyl)benzenesulfonamide (10.2 mg, 0.04 mmol, 1.10 eq.) and 1,8-diazabicyclo[5.4.0]undec-7-ene (10.0 μL, 0.06 mmol, 1.40 eq.) were sequentially added. The resulting mixture was stirred at room temperature for one hour and then concentrated down under reduced pressure. The residue was purified by reverse phase preparative column chromatography (Waters XBridge PREP C₁₈, 5 μM OBD, 19×250 mm, 47-55% acetonitrile/water gradient buffered with 0.1% formic acid) to give the title compound as a yellow solid (3.7 mg). LC-MS (method 5): Rₜ=2.62 min; MS (ESIpos): m/z=449 [M+H]⁺

### Example 234

### 6-(Azetidin-1-yl)-N-[(5-cyclopropyl-2-methoxyphenyl)sulfonyl]-4-fluorobenzofuran-2-carboxamide

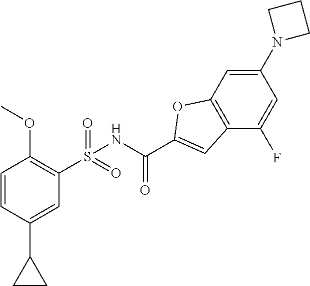 [SMILES: COc1ccc(C2CC2)cc1S(=O)(=O)NC(=O)c1cc2c(F)cc(N3CCC3)cc2o1]

To a room temperature stirred solution of 6-(azetidin-1-yl)-4-fluoro-benzofuran-2-carboxylic acid (40.0 mg, 0.17 mmol, 1.00 eq.) in anhydrous tetrahydrofuran (0.85 mL, 0.20 M) was added carbonyldiimidazole (33.1 mg, 0.20 mmol, 1.20 eq.). After stirring for 1 hour, 5-cyclopropyl-2-methoxy-benzenesulfonamide (42.5 mg, 0.19 mmol, 1.10 eq.) and 1,8-diazabicyclo[5.4.0]undec-7-ene (40.0 μL, 0.24 mmol, 1.40 eq.) were sequentially added. The resulting mixture was stirred at room temperature for one hour and then concentrated under reduced pressure. The residue was purified by reverse phase preparative column chromatography (Waters XBridge PREP C₁₈, 5 μM OBD, 30×150 mm, 54-62% acetonitrile/water gradient buffered with 0.1% formic acid) to give the title compound as a white solid (32 mg). ¹H NMR (400 MHz, DMSO-d₆) δ 12.50 (s, 1H), 8.01 (s, 1H), 7.66-7.60 (m, 1H), 7.37-7.29 (m, 1H), 7.11 (d, 1H), 6.38-6.29 (m, 2H), 3.89 (t, 4H), 3.82 (s, 3H), 2.33 (p, 2H), 2.06-1.96 (m, 1H), 1.03-0.91 (m, 2H), 0.69-0.60 (m, 2H); LC-MS (method 5): Rₜ=2.91 min; MS (ESIpos): m/z=445 [M+H]⁺

### Example 235

### 6-(Azetidin-1-yl)-N-((5-cyclopropyl-2-ethoxyphenyl)sulfonyl)-4-fluorobenzofuran-2-carboxamide

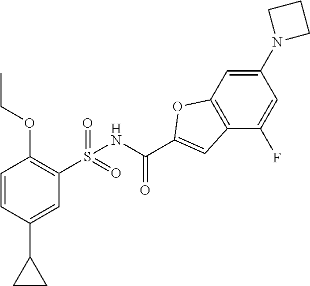 [SMILES: CCOc1ccc(C2CC2)cc1S(=O)(=O)NC(=O)c1cc2c(F)cc(N3CCC3)cc2o1]

To a room temperature stirred solution of 6-(azetidin-1-yl)-4-fluoro-benzofuran-2-carboxylic acid (63.5 mg, 0.27 mmol, 1.00 eq.) in anhydrous tetrahydrofuran (1.35 mL, 0.20 M) was added carbonyldiimidazole (52.5 mg, 0.32 mmol, 1.20 eq.). After stirring for 1 hour, 5-cyclopropyl-2-ethoxy-benzenesulfonamide (71.7 mg, 0.30 mmol, 1.10 eq.) and 1,8-diazabicyclo[5.4.0]undec-7-ene (60.0 μL, 0.38 mmol, 1.40 eq.) were sequentially added. The resulting mixture was stirred at room temperature for 16 h and then concentrated down reduced pressure. The residue was purified by reverse phase preparative column chromatography (Waters XBridge PREP C₁₈, 5 μM OBD, 30×150 mm, 58-66% acetonitrile/water gradient buffered with 0.1% formic acid) to give the title compound as a white solid (32 mg). ¹H NMR (400 MHz, DMSO-d₆) δ 12.39 (s, 1H), 8.05-8.00 (m, 1H), 7.65-7.59 (m, 1H), 7.33-7.25 (m, 1H), 7.13-7.05 (m, 1H), 6.38-6.28 (m, 2H), 4.10 (q, 2H), 3.89 (t, 4H), 2.38-2.26 (m, 2H), 2.06-1.94 (m, 1H), 1.21 (t, 3H), 1.01-0.90 (m, 2H), 0.68-0.59 (m, 2H); LC-MS (method 5): Rₜ=3.07 min; MS (ESIpos): m/z=459 [M+H]⁺

### Example 236

### 6-(Azetidin-1-yl)-4-fluoro-N-[(2-methoxy-5-(2-oxopropyl)phenyl)sulfonyl]benzofuran-2-carboxamide

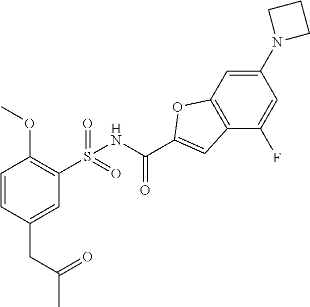 [SMILES: COc1ccc(CC(C)=O)cc1S(=O)(=O)NC(=O)c1cc2c(F)cc(N3CCC3)cc2o1]

To a room temperature stirred solution of 6-(azetidin-1-yl)-4-fluoro-benzofuran-2-carboxylic acid (94.1 mg, 0.40 mmol, 1.00 eq.) in anhydrous tetrahydrofuran (2.00 mL, 0.20 M) was added carbonyldiimidazole (77.8 mg, 0.48 mmol, 1.20 eq.). After stirring for 1 hour, 5-acetonyl-2-methoxy-benzenesulfonamide (107 mg, 0.44 mmol, 1.10 eq.) and 1,8-diazabicyclo[5.4.0]undec-7-ene (80.0 μL, 0.56 mmol, 1.40 eq.) were sequentially added. The resulting mixture was stirred at room temperature for 15 h and then concentrated under reduced pressure. The residue was purified by reverse phase preparative column chromatography (Waters XBridge PREP C₁₈, 5 μM OBD, 30×150 mm, 45-53% acetonitrile/water gradient buffered with 0.1% formic acid) to give the title compound as a yellow solid (103 mg). ¹H NMR (400 MHz, DMSO-d₆) δ 12.50 (s, 1H), 8.03 (s, 1H), 7.76-7.70 (m, 1H), 7.49-7.41 (m, 1H), 7.21-7.14 (m, 1H), 6.37-6.29 (m, 2H), 3.93-3.82 (m, 9H), 2.32 (p, 2H), 2.16 (s, 3H); LC-MS (method 5): Rₜ=2.56 min; MS (ESIpos): m/z=461 [M+H]⁺

### Example 237

### tert-butyl 4-((2-(N-(6-(azetidin-1-yl)-4-fluorobenzofuran-2-carbonyl)sulfamoyl)-4-(tert-butyl)phenoxy)methyl)piperidine-1-carboxylate

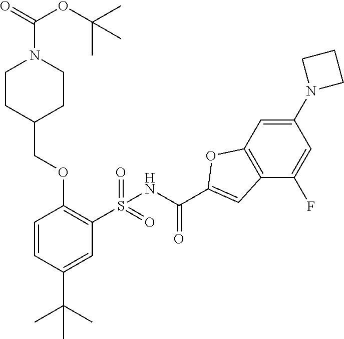 [SMILES: CC(C)(C)OC(=O)N1CCC(COc2ccc(C(C)(C)C)cc2S(=O)(=O)NC(=O)c2cc3c(F)cc(N4CCC4)cc3o2)CC1]

To a room temperature stirred solution of 6-(azetidin-1-yl)-4-fluoro-benzofuran-2-carboxylic acid (14.1 mg, 0.06 mmol, 1.00 eq.) in anhydrous tetrahydrofuran (0.30 mL, 0.20 M) was added carbonyldiimidazole (11.7 mg, 0.07 mmol, 1.20 eq.). After stirring for 1 h, tert-butyl 4-[(4-tert-butyl-2-sulfamoyl-phenoxy)methyl]piperidine-1-carboxylate (28.2 mg, 0.07 mmol, 1.10 eq.) and 1,8-diazabicyclo[5.4.0]undec-7-ene (10.0 μL, 0.08 mmol, 1.40 eq.) were sequentially added. The resulting mixture was stirred at room temperature for 15 h and then concentrated under reduced pressure. The residue was purified by reverse phase preparative column chromatography (Waters XBridge PREP C₁₈, 5 μM OBD, 19×250 mm, 72-80% acetonitrile/water gradient buffered with 0.1% formic acid) to give the title compound as a yellow solid (18 mg). LC-MS (method 5): Rₜ=3.61 min; MS (ESIpos): m/z=644 [M+H]⁺

### Experimental Section—Biological Assays

Examples were tested in selected biological assays one or more times. When tested more than once, data are reported as either average values or as median values, wherein
the average value, also referred to as the arithmetic mean value, represents the sum of the values obtained divided by the number of times tested, and
the median value represents the middle number of the group of values when ranked in ascending or descending order. If the number of values in the data set is odd, the median is the middle value. If the number of values in the data set is even, the median is the arithmetic mean of the two middle values.

Examples were synthesized one or more times. When synthesized more than once, data from biological assays represent average values or median values calculated utilizing data sets obtained from testing of one or more synthetic batch.

An empty field in any of the following tables means that the respective compound has not been tested in that Assay.

The in vitro activity of the compounds of the present invention can be demonstrated in the following assays:

### Kat6a Activity Assay

Kat6a inhibitory activities of the compounds described in the present invention were quantified using a Fluorescence Resonance Energy Transfer (TR-FRET) assay which measures acetylation of a synthetic, biotinylated Histone-H4-derived peptide by the enzyme.

Recombinant human His-tagged Kat6a protein (amino acids 194-810), was purified in-house from Baculovirus- infected insect cells (Sf9). Histone H4 peptide (amino acids 1-24, SGRGKGGKGLGKGGAKRHRK-VLRD-K(Btn)-amide), which was used as substrate, was synthesized by Biosyntan GmbH, Berlin, Germany. Acetyl Coenzyme A was purchased from Sigma-Aldrich (#A-2056).

Kat6a was incubated for 60 mins at 22° C. in the presence of different concentrations of test substances (0 μM, and within the range 0.01-20 μM) in assay buffer [25 mM Tris/HCl pH 8, 1 mM EGTA, 2.5 mM Glutathion, 0.02% Chicken Albumin, 0.05% Pluronic F127, 25 mM NaCl, 220 nM H4 peptide and 600 nM Acetyl Coenzyme A].

The reaction was stopped by addition of Detection Solution (25 mM HEPES pH 7.5, 0.1% BSA, 22 nM SAXL665 (Cisbio #610SAXLE), 100 μM Anacardic Acid (Enzo #ALX-270-381), 1 nM Anti-Histone H4 (ACETYL K8) Antibody (ABCAM #AB15823) and 0.5 nM Anti-Rabbit IgG Eu (Perkin Elmer #AD0083).

### Kat6b Activity Assay

Kat6b inhibitory activities of the compounds described in the present invention were quantified using a Fluorescence Resonance Energy Transfer (TR-FRET) assay which measures acetylation of a synthetic, biotinylated Histone-H4-derived peptide by the enzyme.

Recombinant human GST-tagged Kat6b protein (431-end, N-terminal GST-tag), purified from Baculovirus- infected insect cells (Sf9), was purchased from SignalChem (#K315-381BG). Histone H4 peptide (amino acids 1-24, SGRGKGGKGLGKGGAKRHRK-VLRD-K(Btn)-amide), which was used as substrate, was synthesized by Biosyntan GmbH, Berlin, Germany. Acetyl Coenzym A was purchased from Sigma-Aldrich (#A-2056).

Kat6b was incubated for 30 mins at 22° C. in the presence of different concentrations of test substances (0 μM, and within the range 0.01-20 μM) in assay buffer [25 mM Tris/HCl pH 8, 1 mM EGTA, 2.5 mM Glutathion, 0.02% Chicken Albumin, 0.05% Pluronic F127, 25 mM NaCl, 500 nM H4 peptide and 600 nM Acetyl Coenzyme A].

The reaction was stopped by addition of Detection Solution (25 mM HEPES pH 7.5, 0.1% BSA, 22 nM SAXL665 (Cisbio #610SAXLE), 100 μM Anacardic Acid (Enzo #ALX-270-381), 1 nM Anti-Histone H4 (ACETYL K8) Antibody (ABCAM #AB15823), 0.5 nM Anti-Rabbit IgG Eu (Perkin Elmer #AD0083).

The fluorescence emission at 620 nm and 665 nm after excitation at 330-350 nm was measured in a TR-FRET measuring instrument, for instance a Rubystar or a Pherastar (both from BMG Lab Technologies, Offenburg, Germany) or a Viewlux (Perkin-Elmer). The ratio of the emission at 665 nm and at 622 nm was used as indicator of the amount of acetylated peptide.

The resulting data (ratio) were normalized, taking 0% inhibition as the mean value of control measurements where all reagents were included. In this case 50 nl DMSO were used instead of compounds. A 100% inhibition corresponded to the mean value of control measurements where all reagents except enzyme were included. IC50 values were determined by regression analysis based on a 4 parameter equation (minimum, maximum, ICM, Hill; Y=Max+(Min−Max)/(1+(X/ICso) Hill) using the Screener Software (Genedata).

Table 2 depicts the IC₅₀ values of selected examples in biochemical KAT6A and KAT6B3 assays.

TABLE 2
IC₅₀ values of selected examples in
biochemical KAT6A and KAT6B assays.

| | KAT6A - DPF/HAT domain | KAT6B activity assay |
|---|---|---|
| Example | IC₅₀ [mol/l] (arithmetic mean) | IC₅₀ [mol/l] (arithmetic mean) |
| 1 | 8.61E−9 | 2.09E−8 |
| 2 | 4.52E−8 | 9.52E−8 |
| 3 | 8.10E−9 | |
| 4 | 8.72E−9 | |
| 5 | 1.42E−9 | |
| 6 | 3.49E−8 | |
| 7 | 4.15E−9 | |
| 8 | 2.10E−8 | |
| 9 | 2.12E−8 | |
| 10 | 7.12E−9 | |
| 11 | 7.52E−8 | |
| 12 | 4.27E−8 | |
| 13 | 1.89E−8 | |
| 14 | 2.81E−8 | |
| 15 | 4.72E−8 | |
| 16 | 4.35E−8 | |
| 17 | 4.47E−9 | |
| 18 | 2.42E−8 | |
| 19 | 1.88E−8 | |
| 20 | 3.42E−8 | |
| 21 | 5.51E−8 | |
| 22 | 3.40E−8 | |
| 23 | 2.86E−8 | |
| 24 | 4.67E−8 | |
| 25 | 1.48E−7 | |
| 26 | 1.45E−7 | 2.92E−7 |
| 27 | 4.96E−8 | |
| 28 | 1.36E−7 | |
| 29 | 4.97E−8 | |
| 30 | 2.47E−8 | |
| 31 | 3.49E−8 | |
| 32 | 3.42E−8 | |
| 33 | 7.35E−8 | |
| 34 | 5.96E−8 | |
| 35 | 4.21E−8 | |
| 36 | 3.67E−7 | |
| 37 | 1.81E−7 | |
| 38 | 8.55E−8 | |
| 39 | 7.93E−8 | |
| 40 | 6.42E−8 | |
| 41 | 9.01E−8 | |
| 42 | 1.79E−7 | |
| 43 | 2.18E−7 | |
| 44 | 1.89E−7 | |
| 45 | 1.11E−7 | |
| 46 | 1.22E−7 | |
| 47 | 1.09E−7 | |
| 48 | 2.81E−7 | |
| 49 | 6.73E−8 | |
| 50 | 9.87E−8 | |
| 51 | 1.89E−7 | |
| 52 | 1.01E−7 | |
| 53 | 6.98E−7 | |
| 54 | 1.35E−7 | |
| 55 | 1.06E−7 | |
| 56 | 1.22E−7 | |
| 57 | 1.20E−7 | |
| 58 | 3.15E−7 | |
| 59 | 2.08E−7 | |
| 60 | 4.76E−7 | |
| 61 | 3.99E−7 | |
| 62 | 1.96E−7 | |
| 63 | 1.31E−7 | |
| 64 | 8.51E−8 | |
| 65 | 5.71E−8 | |
| 66 | 1.35E−7 | |
| 67 | 3.45E−7 | |
| 68 | 6.52E−7 | |
| 69 | 3.25E−7 | |
| 70 | 4.94E−7 | |
| 71 | 8.43E−8 | |
| 72 | 1.59E−7 | |
| 73 | 1.05E−7 | |
| 74 | 8.98E−8 | |
| 75 | 3.37E−7 | |
| 76 | 1.11E−7 | |
| 77 | 2.07E−7 | |
| 78 | 1.41E−9 | |
| 79 | 9.58E−9 | 1.36E−8 |
| 80 | 1.21E−8 | |
| 81 | 1.80E−8 | |
| 82 | 1.93E−8 | |
| 83 | 2.67E−8 | |
| 84 | 4.27E−8 | |
| 85 | 4.57E−8 | |
| 86 | 5.48E−8 | |
| 87 | 6.45E−8 | |
| 88 | 1.27E−7 | |
| 89 | 2.69E−7 | |
| 90 | 3.00E−7 | |
| 91 | 3.79E−7 | |
| 92 | 4.64E−7 | |
| 93 | 1.16E−6 | |
| 94 | 1.51E−6 | |
| 95 | 1.52E−6 | |
| 96 | 6.25E−6 | |
| 97 | 1.40E−5 | |
| 98 | >2.00E−5 | |
| 99 | 2.72E−7 | |
| 100 | 1.39E−7 | |
| 101 | 1.21E−7 | |
| 102 | 1.16E−7 | |
| 103 | 6.13E−7 | |
| 104 | 7.93E−7 | |
| 105 | 1.12E−6 | |
| 106 | >2.00E−5 | |
| 107 | 2.75E−7 | 8.53E−7 |
| 108 | 6.86E−8 | |
| 109 | 1.72E−7 | |
| 110 | 2.08E−7 | |
| 111 | 3.08E−6 | |
| 112 | 5.21E−6 | |
| 113 | 4.48E−9 | 1.16E−8 |
| 114 | 4.06E−7 | |
| 115 | 5.63E−7 | |
| 116 | 2.29E−6 | |
| 117 | 1.29E−5 | |
| 118 | 1.99E−6 | |
| 119 | 1.10E−6 | |
| 120 | 6.52E−6 | |
| 121 | 1.34E−5 | |
| 122 | >2.00E−5 | |
| 123 | >2.00E−5 | |
| 124 | >2.00E−5 | |
| 125 | >2.00E−5 | |
| 126 | 5.65E−8 | 2.85E−7 |
| 127 | 4.53E−9 | 5.67E−9 |
| 128 | 8.15E−9 | 1.51E−8 |
| 129 | 3.64E−8 | 5.18E−8 |
| 130 | 1.37E−8 | 1.96E−8 |
| 131 | 2.34E−8 | 2.32E−8 |
| 132 | 1.41E−8 | |
| 133 | 2.18E−8 | |
| 134 | 1.83E−8 | |
| 135 | 1.65E−8 | |
| 136 | 4.56E−8 | |
| 137 | 2.62E−8 | |
| 138 | 6.94E−8 | |
| 139 | 4.13E−8 | |
| 140 | 2.76E−8 | |
| 141 | 1.39E−7 | |
| 142 | 1.49E−7 | |
| 143 | 2.26E−7 | |
| 144 | 5.48E−7 | |
| 145 | 4.02E−7 | |
| 146 | 2.90E−7 | |
| 147 | 9.15E−8 | |
| 148 | 2.74E−7 | |
| 149 | 2.61E−7 | |
| 150 | 4.25E−9 | 9.78E−9 |
| 151 | 5.81E−9 | 1.37E−8 |
| 152 | 1.39E−8 | 2.03E−8 |
| 153 | 1.05E−8 | |
| 154 | 1.34E−8 | |
| 155 | 3.67E−8 | |
| 156 | 1.99E−8 | |
| 157 | 3.73E−8 | |
| 158 | 2.57E−8 | |
| 159 | 3.43E−8 | |
| 160 | 2.03E−7 | |
| 161 | 1.09E−6 | |
| 162 | 1.99E−6 | |
| 163 | 8.62E−7 | |
| 164 | 2.51E−7 | |
| 165 | 3.18E−7 | |
| 166 | 3.62E−7 | |
| 167 | 5.17E−7 | |
| 168 | 6.71E−7 | |
| 169 | 4.07E−6 | |
| 170 | 1.05E−8 | |
| 171 | 1.57E−8 | |
| 172 | 3.40E−8 | |
| 173 | 6.57E−8 | |
| 174 | 2.65E−8 | |
| 175 | 4.49E−8 | |
| 176 | 2.68E−8 | |
| 177 | 2.14E−8 | |
| 178 | 3.53E−8 | |
| 179 | 6.66E−8 | |
| 180 | 3.59E−8 | |
| 181 | 8.95E−8 | |
| 182 | 5.51E−8 | |
| 183 | 8.28E−8 | |
| 184 | 2.94E−7 | |
| 185 | 1.95E−7 | |
| 186 | 1.63E−6 | |
| 187 | 1.53E−6 | |
| 188 | 1.32E−7 | |
| 189 | 8.59E−9 | |
| 190 | 7.29E−8 | |
| 191 | 3.01E−7 | |
| 192 | 2.00E−6 | |
| 193 | 4.82E−6 | |
| 194 | 4.19E−9 | |
| 195 | 5.07E−9 | 2.01E−8 |
| 196 | 8.25E−9 | |
| 197 | 6.06E−9 | |
| 198 | 1.16E−8 | |
| 199 | 3.23E−8 | |
| 200 | 3.41E−8 | |
| 201 | 8.14E−10 | |
| 202 | 8.45E−8 | |
| 203 | 1.56E−7 | |
| 204 | 7.70E−9 | |
| 205 | 7.97E−9 | |
| 206 | 1.10E−7 | |
| 207 | 1.86E−9 | 5.65E−9 |
| 208 | 4.54E−8 | |
| 209 | 2.65E−8 | |
| 210 | 5.26E−8 | |
| 211 | 7.58E−9 | |
| 212 | 3.64E−8 | |
| 213 | 6.18E−9 | 1.47E−8 |
| 214 | 3.91E−9 | 2.03E−8 |
| 215 | 7.18E−9 | |
| 216 | 9.56E−6 | |
| 217 | 1.19E−6 | |
| 218 | 2.71E−7 | |
| 219 | 1.85E−6 | |
| 220 | 9.82E−7 | |
| 221 | 6.15E−8 | |
| 222 | 1.29E−7 | |
| 223 | 1.89E−7 | |
| 224 | 3.72E−9 | 9.82E−9 |
| 225 | 3.72E−9 | 9.82E−9 |
| 226 | 1.79E−9 | |
| 227 | 5.40E−9 | |
| 228 | 2.57E−9 | 1.34E−8 |
| 229 | 4.79E−9 | 2.76E−8 |
| 230 | 7.71E−9 | |
| 231 | 4.22E−9 | 2.88E−8 |
| 232 | | |
| 233 | | |
| 234 | | |
| 235 | | |
| 236 | | |
| 237 | | |

Repression of ESR1 Transcription Upon Treatment of Cells with Compounds

KAT6A controls transcription of the ESR1 gene in breast cancer cells. Compound treatment that interferes with this mechanism was quantified using MVLN cells. These cells constitutively express the ER and are stably transfected with the luciferase (LUC) reporter gene and the corresponding hormone responsive element derived from the 5′-flanking region of the Xenopus Vitellogenin A2 gene (Pons et al., 1990, Demirpense et al., 1993, Joyeux et al., 1993). The repressive effect on ERα transcription activity of a test chemical is directly related to the luciferase measured in the lysate of treated MVLN cells. For measurements the MVLN cells were plated (20,000 cells per well in a 96-well format) in 100 μl culture medium (RPMI 1640 Medium without phenol red (Life Technologies; 11835-063), Pen Strep (Gibco, 15140-122), 10% FCS (Sigma; F2442)) for 24 h in humidified incubator at 37 C and 5% CO₂. Then medium was replaced by 100 μl assay medium (RPMI 1640 Medium without phenol red (Life Technologies; 11835-063), Pen Strep (Gibco, 15140-122), 2.5% FCS (Sigma; F2442)) and cells were treated with compounds (stock solution, 10 mM in DMSO) using the HP D300 Digital Dispenser in a concentration range of 2×10E−5 M to 4.69×10E−9 M in a single-dot curve with 8 dilutions and a DMSO concentration of 0.2%. Each sample was prepared as duplicate and rim wells were excluded. For luciferase signal determination 100 μl of a 1:1 mixture of Steadylite plus (Perkin Elmer; 6016759) and assay medium was added after 24 h for 15 minutes and measured in Victor X3 (Perkin Elmer). To exclude any effects caused by cell loss during the treatment, cell density was determined using Alamar Blue (Invitrogen; DAL1100). Prior to the luciferase measurement 10 μl Alamar Blue were added to each well and the plates were incubated for 2 h in the incubator. Then fluorescence was measured with Victor X3 (Perkin Elmer) at 530 nm/590 nm.

For the evaluation of the results, the values were normalized to DMSO-only treated cells and the Bella DRC Master Sheet was used to calculate EC50 s (EC₅₀ is the concentration for 50% of maximal reduction of luciferase signal achieved by Fulvestrant).

Table 3 shows the results of the inhibition of luciferase signal in MVLN cells upon treatment with the compounds of the invention.

### Proliferation Assays for Compound Characterization

For compound characterization cell survival assays were routinely run in two cell lines. ZR-75-1 is a breast carcinoma cell line containing a focal KAT6A amplification and high KAT6A protein expression. Proliferation of this cell line is inhibited upon treatment with KAT6A inhibitors. As a negative control cell line MDA-MB-436 were used. These cells carry a heterogeneous deletion of the KAT6A gene, have a low KAT6A protein expression and are not growth inhibited upon treatment with KAT6A inhibitors.

For measurements cell lines were plated at the below indicated concentration per 100 mL in 96-well plates in a humidified incubator at 37° C. and 5% CO2.

ZR-75-1 (ATCC, CRL-1500; breast carcinoma); 3000 cells per well; culture medium: RPMI 1640 (Biochrom #FG 1215) medium containing 2.5% FBS (Biochrom #S0615).

MDA-MB-436 (CLS, 300278; breast adenocarcinoma); 3000 cells per well; culture medium: DMEM/Ham's F12 (Biochrom #FG 4815) medium containing 2.5% FBS (Biochrom #S0615).

24 hours later, cells were treated with compounds (stock solution, 10 mM in DMSO) using the HP D300 Digital Dispenser in a concentration range of 2.0×10E−5 M to 2.0×10E−9 M in a single-dot curve with 9 dilutions and a DMSO concentration of 0.2%. Each sample was prepared as triplicate and rim wells were excluded. Plates were incubated at 37° C. and 5% CO₂ for 144 h. Cell viability was measured by adding 10 μL of Alamar Blue Reagent (Invitrogen #DAL 1100) and plates were incubated for 2 h at 37° C. and 5% CO2. Fluorescence was measured at 590 nM wavelength with a Tecan Spark or Victor X3 MTP-Reader (PerkinElmer).

For the evaluation of the results, the values were normalized to DMSO-only treated cells and the Bella DRC Master Sheet was used to calculate EC50 s (EC₅₀ is the concentration for 50% of maximal inhibition of cell proliferation).

Table 3 shows the results of the inhibition in proliferation assays performed in ZR-75-1 and MDA-MB-436 cells upon treatment with the compounds of the invention.

TABLE 3
IC₅₀ values of selected examples in proliferation assays
performed in MVLN, ZR-75-1 and MDA-MB-436 cells.

| | | ZR75-1 | LCLC97TM1 | MDA-MB-436 |
|---|---|---|---|---|
| | MVLN reporter | proliferation | proliferation | proliferation |
| | assay; IC50 | assay; EC50 | assay; EC50 | assay; EC50 |
| | [M] | [M] | [M] | [M] |
| Exam- | (arithmetic | (arithmetic | (arithmetic | (arithmetic |
| ple | mean) | mean) | mean) | mean) |
| 1 | 3.89E−8 | 7.18E−8 | 2.13E−7 | >2.00E−5 |
| 2 | 5.54E−8 | 1.64E−7 | 3.38E−7 | >2.00E−5 |
| 3 | 5.25E−8 | 2.32E−7 | 1.11E−6 | >2.00E−5 |
| 4 | 6.30E−8 | 1.14E−7 | 6.42E−7 | >2.00E−5 |
| 5 | 6.43E−8 | 3.57E−6 | 9.88E−6 | >2.00E−5 |
| 6 | 7.45E−8 | 3.28E−8 | 3.12E−7 | >2.00E−5 |
| 7 | 1.23E−7 | 3.70E−7 | 2.92E−7 | >2.00E−5 |
| 8 | 1.25E−7 | | | |
| 9 | 1.34E−7 | | | |
| 10 | 1.36E−7 | 2.21E−7 | 6.61E−7 | >2.00E−5 |
| 11 | 1.45E−7 | | | |
| 12 | 1.63E−7 | | | |
| 13 | 1.66E−7 | | | |
| 14 | 6.93E−8 | 9.93E−8 | 1.39E−6 | >2.00E−5 |
| 15 | 7.55E−8 | 2.46E−7 | 6.28E−7 | >2.00E−5 |
| 16 | 1.69E−7 | | | |
| 17 | 1.76E−7 | 2.76E−7 | 8.49E−7 | >2.00E−5 |
| 18 | 1.92E−7 | | | |
| 19 | 2.06E−7 | | | >2.00E−5 |
| 20 | 3.03E−7 | 2.87E−7 | 1.42E−6 | >2.00E−5 |
| 21 | 2.98E−7 | | | |
| 22 | 3.19E−7 | | | |
| 23 | 2.72E−7 | | | |
| 24 | 3.00E−7 | | | |
| 25 | 3.26E−7 | | | |
| 26 | 3.85E−7 | 5.06E−7 | 3.90E−6 | >2.00E−5 |
| 27 | 3.54E−7 | | | |
| 28 | 3.56E−7 | | | |
| 29 | 3.79E−7 | | | |
| 30 | 4.18E−7 | | | |
| 31 | 5.02E−7 | | | |
| 32 | 5.11E−7 | | | |
| 33 | 5.37E−7 | | | |
| 34 | 5.79E−7 | | | |
| 35 | 6.09E−7 | | | |
| 36 | 6.04E−7 | | | |
| 37 | 7.04E−7 | | | |
| 38 | 7.07E−7 | | | |
| 39 | 7.88E−7 | | | |
| 40 | 7.89E−7 | | | |
| 41 | 8.57E−7 | | | |
| 42 | 8.64E−7 | | | |
| 43 | 9.91E−7 | | | |
| 44 | 1.01E−6 | | | |
| 45 | 1.10E−6 | | | |
| 46 | 1.15E−6 | | | |
| 47 | 1.30E−6 | | | |
| 48 | 1.29E−6 | | | |
| 49 | 1.41E−6 | | | |
| 50 | 1.42E−6 | | | |
| 51 | 1.51E−6 | | | |
| 52 | 1.55E−6 | | | |
| 53 | 1.58E−6 | | | |
| 54 | 1.72E−6 | | | |
| 55 | 1.69E−6 | | | |
| 56 | 2.11E−6 | | | |
| 57 | 2.50E−6 | | | |
| 58 | 3.87E−6 | | | |
| 59 | 4.29E−6 | | | |
| 60 | 5.29E−6 | | | |
| 61 | 5.60E−6 | | | |
| 62 | 6.43E−6 | | | |
| 63 | 6.48E−6 | | | |
| 64 | 8.00E−6 | | | |
| 65 | 8.32E−6 | | | |
| 66 | 1.00E−5 | | | |
| 67 | 1.10E−5 | | | |
| 68 | 1.26E−5 | | | |
| 69 | 1.56E−5 | | | |
| 70 | 1.58E−5 | | | |
| 71 | 1.72E−5 | | | |
| 72 | 1.89E−5 | | | |
| 73 | 2.56E−6 | | | |
| 74 | 2.56E−6 | | | |
| 75 | 2.76E−6 | | | |
| 76 | 2.82E−6 | | | |
| 77 | 3.45E−6 | | | |
| 78 | 4.95E−9 | 5.87E−8 | 5.75E−8 | >2.00E−5 |
| 79 | 2.10E−8 | 3.61E−8 | 3.90E−8 | >2.00E−5 |
| 80 | 7.27E−8 | 1.49E−7 | 3.17E−7 | >2.00E−5 |
| 81 | 1.57E−7 | | | |
| 82 | 3.66E−7 | | | |
| 83 | 2.85E−7 | | | |
| 84 | 5.73E−7 | | | |
| 85 | 3.10E−7 | | | |
| 86 | 3.33E−7 | | | |
| 87 | 1.20E−6 | | | |
| 88 | 4.45E−7 | | | |
| 89 | >2.00E−5 | | | |
| 90 | >2.00E−5 | | | |
| 91 | 3.08E−7 | | | |
| 92 | >2.00E−5 | | | |
| 93 | >2.00E−5 | | | |
| 94 | >2.00E−5 | | | |
| 95 | >2.00E−5 | | | |
| 96 | >2.00E−5 | | | |
| 97 | >2.00E−5 | | | |
| 98 | >2.00E−5 | | | |
| 99 | 8.10E−7 | | | |
| 100 | 1.29E−6 | | | |
| 101 | 1.94E−6 | | | |
| 102 | 4.07E−6 | | | |
| 103 | 1.02E−5 | | | |
| 104 | 1.05E−5 | | | |
| 105 | >2.00E−5 | | | |
| 106 | >2.00E−5 | | | |
| 107 | 1.33E−7 | 4.10E−7 | 9.16E−7 | |
| 108 | 1.29E−7 | | | |
| 109 | 5.92E−7 | | | |
| 110 | 5.47E−7 | | | |
| 111 | >2.00E−5 | | | |
| 112 | >2.00E−5 | | | |
| 113 | 1.08E−8 | 2.94E−8 | 3.70E−8 | >2.00E−5 |
| 114 | >2.00E−5 | | | |
| 115 | 3.81E−6 | | | |
| 116 | 1.45E−6 | 5.72E−6 | >2.00E−5 | |
| 117 | 1.73E−5 | | | |
| 118 | 1.20E−6 | 2.46E−6 | 2.72E−6 | >2.00E−5 |
| 119 | 3.04E−6 | 3.70E−6 | 1.46E−5 | >2.00E−5 |
| 120 | >2.00E−5 | | | >2.00E−5 |
| 121 | >2.00E−5 | | | |
| 122 | >2.00E−5 | | | >2.00E−5 |
| 123 | >2.00E−5 | | | |
| 124 | >2.00E−5 | | | >2.00E−5 |
| 125 | >2.00E−5 | >2.00E−5 | >2.00E−5 | >2.00E−5 |
| 126 | 9.43E−8 | 3.07E−7 | 1.12E−6 | >2.00E−5 |
| 127 | 3.54E−9 | 2.55E−8 | 5.73E−8 | >2.00E−5 |
| 128 | 1.90E−8 | 6.73E−8 | 2.20E−7 | >2.00E−5 |
| 129 | 2.74E−8 | 1.42E−7 | 4.11E−7 | >2.00E−5 |
| 130 | 3.23E−8 | 8.86E−8 | 2.36E−7 | >2.00E−5 |
| 131 | 3.83E−8 | 2.09E−7 | 7.53E−8 | >2.00E−5 |
| 132 | 5.41E−8 | 1.08E−7 | 1.36E−7 | 1.22E−5 |
| 133 | 6.41E−8 | 1.61E−7 | 6.64E−7 | >2.00E−5 |
| 134 | 7.44E−8 | 8.18E−8 | 3.76E−7 | >2.00E−5 |
| 135 | 9.74E−8 | | | >2.00E−5 |
| 136 | 1.40E−7 | | | >2.00E−5 |
| 137 | 1.74E−7 | | | |
| 138 | 2.06E−7 | | | >2.00E−5 |
| 139 | 2.36E−7 | | | |
| 140 | 2.91E−7 | | | >2.00E−5 |
| 141 | 5.12E−7 | | | |
| 142 | 1.20E−6 | | | |
| 143 | 4.61E−6 | | | |
| 144 | 7.93E−6 | | | |
| 145 | 8.40E−6 | | | |
| 146 | 2.83E−6 | | | |
| 147 | 2.98E−6 | | | |
| 148 | 3.14E−6 | | | |
| 149 | 3.66E−6 | | | |
| 150 | 5.97E−9 | 5.78E−8 | 4.58E−8 | >2.00E−5 |
| 151 | 1.55E−8 | 5.67E−8 | 6.63E−8 | >2.00E−5 |
| 152 | 3.69E−8 | 7.66E−8 | 1.63E−7 | >2.00E−5 |
| 153 | 4.69E−8 | 4.98E−8 | 1.60E−7 | >2.00E−5 |
| 154 | 7.11E−8 | 4.95E−8 | 2.25E−7 | >2.00E−5 |
| 155 | 7.14E−8 | 7.48E−8 | 4.28E−7 | >2.00E−5 |
| 156 | 7.43E−8 | 1.34E−7 | 6.60E−7 | >2.00E−5 |
| 157 | 7.51E−8 | 1.10E−7 | 7.07E−7 | >2.00E−5 |
| 158 | 1.05E−7 | | | |
| 159 | 7.45E−7 | | | |
| 160 | 1.45E−6 | 4.34E−6 | >2.00E−5 | >2.00E−5 |
| 161 | 5.92E−6 | 7.56E−6 | >2.00E−5 | >2.00E−5 |
| 162 | 1.95E−5 | >2.00E−5 | >2.00E−5 | >2.00E−5 |
| 163 | 3.67E−6 | 6.67E−6 | >2.00E−5 | >2.00E−5 |
| 164 | 2.34E−6 | | | |
| 165 | 8.64E−7 | | | |
| 166 | 7.22E−6 | | | |
| 167 | >2.00E−5 | | | |
| 168 | >2.00E−5 | | | |
| 169 | >2.00E−5 | | | |
| 170 | 1.33E−7 | | | |
| 171 | 6.68E−8 | 3.98E−7 | 4.40E−7 | >2.00E−5 |
| 172 | 1.35E−7 | | | |
| 173 | 1.58E−7 | | | |
| 174 | 1.65E−7 | | | |
| 175 | 1.65E−7 | | | |
| 176 | 1.88E−7 | | | |
| 177 | 2.24E−7 | | | |
| 178 | 3.16E−7 | | | |
| 179 | 5.53E−7 | | | |
| 180 | 6.28E−7 | | | |
| 181 | 6.47E−7 | | | |
| 182 | 8.48E−7 | | | |
| 183 | 9.24E−7 | | | |
| 184 | 5.21E−6 | | | |
| 185 | 9.61E−6 | | | |
| 186 | 1.70E−5 | | | |
| 187 | 1.85E−5 | | | |
| 188 | 2.55E−6 | | | |
| 189 | 1.30E−7 | | | |
| 190 | 1.93E−7 | | | |
| 191 | 4.99E−6 | | | |
| 192 | >2.00E−5 | | | |
| 193 | >2.00E−5 | | | |
| 194 | 7.76E−8 | | | |
| 195 | 1.11E−7 | 1.99E−7 | 8.37E−7 | >2.00E−5 |
| 196 | 4.72E−8 | 1.44E−7 | 8.10E−7 | >2.00E−5 |
| 197 | 1.62E−7 | | | |
| 198 | 2.95E−7 | | | |
| 199 | 5.49E−7 | | | |
| 200 | 5.50E−7 | | | |
| 201 | 6.48E−7 | 3.26E−6 | 2.81E−6 | >2.00E−5 |
| 202 | 1.04E−6 | | | |
| 203 | 3.78E−6 | | | |
| 204 | 1.79E−7 | 3.40E−7 | 8.36E−7 | 1.60E−6 |
| 205 | 7.15E−8 | 1.73E−7 | 9.37E−7 | >2.00E−5 |
| 206 | 4.44E−7 | | | |
| 207 | 9.04E−9 | 1.03E−7 | 4.29E−8 | >2.00E−5 |
| 208 | 7.78E−8 | | | |
| 209 | 5.86E−8 | 4.59E−8 | 6.32E−7 | >2.00E−5 |
| 210 | 1.13E−7 | | | |
| 211 | 2.44E−7 | | | |
| 212 | 1.59E−7 | | | |
| 213 | 3.31E−8 | 1.17E−7 | 1.52E−7 | 1.41E−5 |
| 214 | 2.43E−8 | 4.47E−7 | 5.77E−7 | |
| 215 | 7.43E−8 | 5.82E−7 | 2.83E−6 | >2.00E−5 |
| 216 | >2.00E−5 | | | >2.00E−5 |
| 217 | 1.05E−5 | | | >2.00E−5 |
| 218 | 1.91E−6 | | | >2.00E−5 |
| 219 | 3.86E−6 | | | >2.00E−5 |
| 220 | 6.23E−6 | | | >2.00E−5 |
| 221 | 3.82E−7 | | | >2.00E−5 |
| 222 | 1.44E−6 | | | >2.00E−5 |
| 223 | 7.22E−7 | | | >2.00E−5 |
| 224 | 2.76E−8 | 7.50E−8 | 1.14E−7 | 1.77E−5 |
| 225 | 2.76E−8 | 7.50E−8 | 1.14E−7 | 1.77E−5 |
| 226 | 5.37E−8 | 2.78E−8 | 4.63E−8 | >2.00E−5 |
| 227 | 4.82E−8 | 7.19E−8 | 1.22E−7 | >2.00E−5 |
| 228 | 2.39E−8 | 1.16E−7 | 1.67E−7 | >2.00E−5 |
| 229 | 3.83E−8 | 2.18E−7 | 1.80E−7 | >2.00E−5 |
| 230 | 8.72E−8 | | | |
| 231 | 1.83E−8 | 1.58E−7 | 1.99E−7 | >2.00E−5 |
| 232 | | | | |
| 233 | | | | |
| 234 | | | | |
| 235 | | | | |
| 236 | | | | |
| 237 | | | | |

1. A compound of general formula (I), 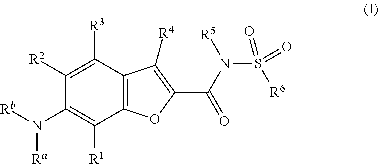
wherein:
R¹ is a hydrogen atom;
R² is a hydrogen atom;
R³ is selected from a hydrogen atom and a fluorine atom;
R⁴ is a hydrogen atom;
R⁵ is a hydrogen atom;
R⁶ is selected from a phenyl group, a naphthyl group and a heteroaryl group,
wherein said phenyl, naphthyl or heteroaryl group is each optionally substituted with one or more substituents independently selected from a halogen atom, a hydroxy group, a C₁-C₆-alkyl group, a C₁-C₆-hydroxyalkyl group, a C₁-C₆-haloalkyl group, a C₁-C₆-alkoxy group, a (C₁-C₃-alkyl)-O—(C₁-C₂-alkyl)- group, a (C₁-C₃-alkyl)-O—(C₁-C₂-alkyl)-O— group, a (C₃-C₈-cycloalkyl)-(C₁-C₂-alkyl)-O— group, a (C₃-C₈-halocycloalkyl)-(C₁-C₂-alkyl)-O— group, a (phenyl)-(C₁-C₂-alkyl)-O— group, a C₁-C₆-haloalkoxy group, a C₂-C₆-alkenyl group, a C₃-C₈-cycloalkyl group, a C₃-C₈-cycloalkoxy group, a C₁-C₆-thioalkyl group, a heterocycloalkyl group, a R⁷R⁸N— group, a (C₁-C₂-alkyl)-S(═O)₂— group, a (phenyl)-S(═O)₂— group, a (R⁷R⁸N)—S(═O)₂— group, a (H₃C)—(C═O)— group, a (R⁷R⁸N)—(C═O)— group, a (R⁷R⁸N)—(C═O)—O— group, a (R⁷R⁸N)—(C═O)— group, a R⁹OOC—(C₁-C₃-alkyl)- group, a R⁹OOC— group, a phenyl group, a (phenyl)-O— group and a heteroaryl group,
wherein said heterocycloalkyl group is connected to the rest of the molecule via a carbon atom of said heterocycloalkyl group,
wherein said phenyl, heteroaryl or heterocycloalkyl group is each optionally substituted with one or more substituents independently selected from a halogen atom, a C₁-C₃-alkyl group, a phenyl group and a R⁹OOC— group;
R⁷ and R⁸ are each independently selected from a hydrogen atom, a C₁-C₆-alkyl group, a C₃-C₈-cycloalkyl group and a phenyl group,
wherein said phenyl group is optionally substituted with one or more substituents independently selected from a halogen atom, a C₁-C₃-alkyl group, a C₁-C₃-haloalkyl group and a C₁-C₃-alkoxy group;
or R⁷ and R⁸ together with the nitrogen atom to which they are attached form a 4- to 7-membered nitrogen-containing heterocycloalkyl group, wherein said heterocycloalkyl group optionally contains one, two or three further heteroatoms independently selected from nitrogen, oxygen and sulfur and is optionally substituted with one or more substituents independently selected from a halogen atom, a C₁-C₃-alkyl group, a C₁-C₃-haloalkyl group, a C₁-C₃-alkoxy group and a phenyl group;
R⁹ is selected from a hydrogen atom, a C₁-C₃-alkyl group and a C₃-C₆-cycloalkyl group;
R^{a} and R^{b} together with the nitrogen atom to which they are attached form a 4- membered nitrogen-containing heterocycloalkyl group which is optionally substituted one, two or three times, each substituent independently selected from a halogen atom, a C₁-C₂-alkyl group, a C₁-C₂-haloalkyl group, a C₁-C₂-alkoxy group, a C₁-C₃-hydroxyalkyl group and a phenyl group;
or a tautomer, or an N-oxide, or a salt thereof, or a salt of a tautomer, or a salt of an N-oxide, or a mixture of same.

2. The compound according to claim 1, wherein:
R¹ is a hydrogen atom;
R² is a hydrogen atom;
R³ is selected from a hydrogen atom and a fluorine atom;
R⁴ is a hydrogen atom;
R⁵ is a hydrogen atom;
R⁶ is selected from a phenyl group, a naphthyl group and a heteroaryl group,
wherein said phenyl, naphthyl or heteroaryl group is each optionally substituted with one or more substituents independently selected from a halogen atom, a hydroxy group, a C₁-C₆-alkyl group, a C₁-C₆-hydroxyalkyl group, a C₁-C₆-haloalkyl group, a C₁-C₆-alkoxy group, a (C₁-C₃-alkyl)-O—(C₁-C₂-alkyl)- group, a (C₁-C₃-alkyl)-O—(C₁-C₂-alkyl)-O— group, a (C₃-C₈-cycloalkyl)-(C₁-C₂-alkyl)-O— group, a (C₃-C₈-halocycloalkyl)-(C₁-C₂-alkyl)-O— group, a (phenyl)-(C₁-C₂-alkyl)-O— group, a C₁-C₆-haloalkoxy group, a C₂-C₆-alkenyl group, a C₃-C₈-cycloalkyl group, a C₃-C₈-cycloalkoxy group, a C₁-C₆-thioalkyl group, a heterocycloalkyl group, a R⁷R⁸N— group, a (C₁-C₂-alkyl)-S(═O)₂— group, a (phenyl)-S(═O)₂— group, a (R⁷R⁸N)—S(═O)₂— group, a (H₃C)—(C═O)— group, a (R⁷R⁸N)—(C═O)— group, a (R⁷R⁸N)—(C═O)—O— group, a (R⁷R⁸N)—(C═O)— group, a R⁹OOC—(C₁-C₃-alkyl)- group, a R⁹OOC— group, a phenyl group, a (phenyl)-O— group and a heteroaryl group,
wherein said heterocycloalkyl group is connected to the rest of the molecule via a carbon atom of said heterocycloalkyl group,
wherein said phenyl, heteroaryl or heterocycloalkyl group is each optionally substituted with one or more substituents independently selected from a halogen atom, a C₁-C₃-alkyl group and a R⁹OOC— group;
R⁷ and R⁸ are each independently selected from a hydrogen atom, a C₁-C₆-alkyl group, a C₃-C₈-cycloalkyl group and a phenyl group,
wherein said phenyl group is optionally substituted with one or more substituents independently selected from a halogen atom, a C₁-C₃-alkyl group, a C₁-C₃-haloalkyl group and a C₁-C₃-alkoxy group;
or R⁷ and R⁸ together with the nitrogen atom to which they are attached form a 4- to 7-membered nitrogen-containing heterocycloalkyl group, wherein said heterocycloalkyl group optionally contains one, two or three further heteroatoms independently selected from nitrogen, oxygen and sulfur and is optionally substituted with one or more substituents independently selected from a halogen atom, a C₁-C₃-alkyl group, a C₁-C₃-haloalkyl group, a C₁-C₃-alkoxy group and a phenyl group;
R⁹ is selected from a hydrogen atom, a C₁-C₃-alkyl group and a C₃-C₆-cycloalkyl group;
R^{a} and R^{b} together with the nitrogen atom to which they are attached form a 4- membered nitrogen-containing heterocycloalkyl group which is optionally substituted one, two or three times, each substituent independently selected from a halogen atom, a C₁-C₂-haloalkyl group and a C₁-C₃-hydroxyalkyl group;
or a tautomer, or an N-oxide, or a salt thereof, or a salt of a tautomer, or a salt of an N-oxide, or a mixture of same.

3. The compound according to claim 2, wherein:
R¹ is a hydrogen atom;
R² is a hydrogen atom;
R³ is selected from a hydrogen atom and a fluorine atom;
R⁴ is a hydrogen atom;
R⁵ is a hydrogen atom;
R⁶ is selected from a phenyl group, a naphthyl group and a heteroaryl group,
wherein said phenyl, naphthyl or heteroaryl group is each optionally substituted with one or more substituents independently selected from a halogen atom, a hydroxy group, a C₁-C₆-alkyl group, a C₁-C₆-hydroxyalkyl group, a C₁-C₆-haloalkyl group, a C₁-C₆-alkoxy group, a (C₁-C₃-alkyl)-O—(C₁-C₂-alkyl)- group, a (C₁-C₃-alkyl)-O—(C₁-C₂-alkyl)-O— group, a (C₃-C₈-cycloalkyl)-(C₁-C₂-alkyl)-O— group, a (C₃-C₈-halocycloalkyl)-(C₁-C₂-alkyl)-O— group, a (phenyl)-(C₁-C₂-alkyl)-O— group, a C₁-C₆-haloalkoxy group, a C₂-C₆-alkenyl group, a C₃-C₈-cycloalkyl group, a C₃-C₈-cycloalkoxy group, a C₁-C₆-thioalkyl group, a heterocycloalkyl group, a R⁷R⁸N— group, a (C₁-C₂-alkyl)-S(═O)₂— group, a (phenyl)-S(═O)₂— group, a (R⁷R⁸N)—S(═O)₂— group, a (H₃C)—(C═O)— group, a (R⁷R⁸N)—(C═O)— group, a (R⁷R⁸N)—(C═O)—O— group, a (R⁷R⁸N)—(C═O)— group, a R⁹OOC—(C₁-C₃-alkyl)- group, a R⁹OOC— group, a phenyl group, a (phenyl)-O— group and a heteroaryl group,
wherein said heterocycloalkyl group is connected to the rest of the molecule via a carbon atom of said heterocycloalkyl group,
wherein said phenyl, heteroaryl or heterocycloalkyl group is each optionally substituted with one or more substituents independently selected from a halogen atom, a C₁-C₃-alkyl group and a R⁹OOC— group;
R⁷ and R⁸ are each independently selected from a hydrogen atom, a C₁-C₆-alkyl group, a C₃-C₈-cycloalkyl group and a phenyl group,
wherein said phenyl group is optionally substituted with one or more substituents independently selected from a halogen atom, a C₁-C₃-alkyl group, a C₁-C₃-haloalkyl group and a C₁-C₃-alkoxy group;
or R⁷ and R⁸ together with the nitrogen atom to which they are attached form a 4- to 7-membered nitrogen-containing heterocycloalkyl group, wherein said heterocycloalkyl group optionally contains one, two or three further heteroatoms independently selected from nitrogen, oxygen and sulfur and is optionally substituted with one or more substituents independently selected from a halogen atom, a C₁-C₃-alkyl group, a C₁-C₃-haloalkyl group, a C₁-C₃-alkoxy group and a phenyl group;
R⁹ is selected from a hydrogen atom, a C₁-C₃-alkyl group and a C₃-C₆-cycloalkyl group;
R^{a} and R^{b} together with the nitrogen atom to which they are attached form a 4- membered nitrogen-containing heterocycloalkyl group which is optionally substituted one, two or three times with a halogen atom;
or a tautomer, or an N-oxide, or a salt thereof, or a salt of a tautomer, or a salt of an N-oxide, or a mixture of same.

4. The compound according to claim 3, wherein:
R¹ is a hydrogen atom;
R² is a hydrogen atom;
R³ is selected from a hydrogen atom and a fluorine atom;
R⁴ is a hydrogen atom;
R⁵ is a hydrogen atom;
R⁶ is selected from a phenyl group, a naphthyl group and a heteroaryl group,
wherein said phenyl, naphthyl or heteroaryl group is each optionally substituted with one or more substituents independently selected from a halogen atom, a C₁-C₆-alkyl group, a C₁-C₆-haloalkyl group, a C₁-C₆-alkoxy group, a (C₁-C₃-alkyl)-O—(C₁-C₂-alkyl)- group, a (C₃-C₈-cycloalkyl)-(C₁-C₂-alkyl)-O— group, a (C₃-C₈-halocycloalkyl)-(C₁-C₂-alkyl)-O— group, a (phenyl)-(C₁-C₂-alkyl)-O— group, a C₁-C₆-haloalkoxy group, a C₂-C₆-alkenyl group, a C₃-C₈-cycloalkoxy group, a C₁-C₆-thioalkyl group, a heterocycloalkyl group, a R⁷R⁸N— group, a (C₁-C₂-alkyl)-S(═O)₂— group, a (R⁷R⁸N)—(C═O)— group, a (R⁷R⁸N)—(C═O)—O— group, a R⁹OOC—(C₁-C₃-alkyl)- group, a R⁹OOC— group, a phenyl group, a (phenyl)-O— group and a heteroaryl group,
wherein said heterocycloalkyl group is connected to the rest of the molecule via a carbon atom of said heterocycloalkyl group,
wherein said phenyl or heteroaryl group is each optionally substituted with one or more substituents independently selected from a halogen atom and a C₁-C₃-alkyl group;
R⁷ and R⁸ are each independently selected from a hydrogen atom, a C₁-C₆-alkyl group, a C₃-C₈-cycloalkyl group and a phenyl group,
wherein said phenyl group is optionally substituted with one or more substituents independently selected from a halogen atom, a C₁-C₃-alkyl group, a C₁-C₃-haloalkyl group and a C₁-C₃-alkoxy group;
or R⁷ and R⁸ together with the nitrogen atom to which they are attached form a 4- to 7-membered nitrogen-containing heterocycloalkyl group, wherein said heterocycloalkyl group optionally contains one, two or three further heteroatoms independently selected from nitrogen, oxygen and sulfur and is optionally substituted with one or more substituents independently selected from a halogen atom, a C₁-C₃-alkyl group, a C₁-C₃-haloalkyl group, a C₁-C₃-alkoxy group and a phenyl group;
R⁹ is selected from a hydrogen atom, a C₁-C₃-alkyl group and a C₃-C₆-cycloalkyl group;
R^{a} and R^{b} together with the nitrogen atom to which they are attached form a 4- membered nitrogen-containing heterocycloalkyl group which is optionally substituted one, two or three times with a halogen atom;
or a tautomer, or an N-oxide, or a salt thereof, or a salt of a tautomer, or a salt of an N-oxide, or a mixture of same.

5. The compound according to claim 4, wherein:
R¹ is a hydrogen atom;
R² is a hydrogen atom;
R³ is selected from a hydrogen atom and a fluorine atom;
R⁴ is a hydrogen atom;
R⁵ is a hydrogen atom;
R⁶ is selected from a phenyl group and a heteroaryl group,
wherein said phenyl or heteroaryl group is each optionally substituted with one or more substituents independently selected from a halogen atom, a C₁-C₆-alkyl group, a C₁-C₆-haloalkyl group, a C₁-C₆-alkoxy group, a (C₃-C₈-cycloalkyl)-(C₁-C₂-alkyl)-O— group, a (C₃-C₈-halocycloalkyl)-(C₁-C₂-alkyl)-O— group, a (phenyl)-(C₁-C₂-alkyl)-O— group, a C₁-C₆-haloalkoxy group, a C₃-C₈-cycloalkoxy group, a heterocycloalkyl group, a R⁷R⁸N— group, a (phenyl)-O— group and a heteroaryl group,
wherein said heterocycloalkyl group is connected to the rest of the molecule via a carbon atom of said heterocycloalkyl group,
wherein said heteroaryl or heterocycloalkyl group is each optionally substituted with one or more substituents independently selected from a C₁-C₃-alkyl group and a phenyl group;
R⁷ and R⁸ are each independently selected from a hydrogen atom, a C₁-C₆-alkyl group, a C₃-C₈-cycloalkyl group and a phenyl group,
wherein said phenyl group is optionally substituted with one or more substituents independently selected from a halogen atom, a C₁-C₃-alkyl group, a C₁-C₃-haloalkyl group and a C₁-C₃-alkoxy group;
or R⁷ and R⁸ together with the nitrogen atom to which they are attached form a 4- to 7-membered nitrogen-containing heterocycloalkyl group, wherein said heterocycloalkyl group optionally contains one, two or three further heteroatoms independently selected from nitrogen, oxygen and sulfur and is optionally substituted with one or more substituents independently selected from a halogen atom, a C₁-C₃-alkyl group, a C₁-C₃-haloalkyl group, a C₁-C₃-alkoxy group and a phenyl group;
R⁹ is selected from a hydrogen atom, a C₁-C₃-alkyl group and a C₃-C₆-cycloalkyl group;
R^{a} and R^{b} together with the nitrogen atom to which they are attached form a 4- membered nitrogen-containing heterocycloalkyl group which is optionally substituted one, two or three times with a halogen atom;
or a tautomer, or an N-oxide, or a salt thereof, or a salt of a tautomer, or a salt of an N-oxide, or a mixture of same.

6. A compound of general formula (I) according to claim 1, which is selected from
6-(Azetidin-1-yl)-N-(2-ethoxy-6-methoxybenzene-1-sulfonyl)-4-fluoro-1-benzofuran-2-carboxamide,
6-(Azetidin-1-yl)-N-(2-ethoxybenzene-1-sulfonyl)-4-fluoro-1-benzofuran-2-carboxamide,
6-(Azetidin-1-yl)-N-[2-(cyclopentyloxy)-5-ethylbenzene-1-sulfonyl]-4-fluoro-1-benzofuran-2-carboxamide,
6-(Azetidin-1-yl)-N-(2-{[butan-2-yl]oxy}-5-methylbenzene-1-sulfonyl)-4-fluoro-1-benzofuran-2-carboxamide,
6-(Azetidin-1-yl)-N-[2-(benzyloxy)-6-(cyclobutyloxy)benzene-1-sulfonyl]-4-fluoro-1-benzofuran-2-carboxamide,
6-(Azetidin-1-yl)-N-(2-ethoxy-5-methylbenzene-1-sulfonyl)-4-fluoro-1-benzofuran-2-carboxamide,
6-(Azetidin-1-yl)-N-[2-(benzyloxy)-6-ethoxybenzene-1-sulfonyl]-4-fluoro-1-benzofuran-2-carboxamide,
6-(Azetidin-1-yl)-4-fluoro-N-(7-methoxyquinoline-8-sulfonyl)-1-benzofuran-2-carboxamide,
6-(Azetidin-1-yl)-N-[2-(cyclopentyloxy)-5-methylbenzene-1-sulfonyl]-4-fluoro-1-benzofuran-2-carboxamide,
6-(Azetidin-1-yl)-N-(2-ethoxy-6-propoxybenzene-1-sulfonyl)-4-fluoro-1-benzofuran-2-carboxamide,
6-(Azetidin-1-yl)-N-[2-(dimethylamino)benzene-1-sulfonyl]-4-fluoro-1-benzofuran-2-carboxamide,
6-(Azetidin-1-yl)-N-(3,4-dihydro-2H-1-benzopyran-8-sulfonyl)-4-fluoro-1-benzofuran-2-carboxamide,
6-(Azetidin-1-yl)-4-fluoro-N-(2-1{[-1,1,1-trifluoropropan-2-yl]oxy}benzene-1-sulfonyl)-1-benzofuran-2-carboxamide (rac),
6-(Azetidin-1-yl)-4-fluoro-N-(2-1{[-1,1,1-trifluoropropan-2-yl]oxy}benzene-1-sulfonyl)-1-benzofuran-2-carboxamide (Enantiomer 1),
6-(Azetidin-1-yl)-4-fluoro-N-(2-1{[-1,1,1-trifluoropropan-2-yl]oxy}benzene-1-sulfonyl)-1-benzofuran-2-carboxamide (Enantiomer 2),
6-(Azetidin-1-yl)-N-[2-(cyclobutyloxy)-6-fluorobenzene-1-sulfonyl]-4-fluoro-1-benzofuran-2-carboxamide,
6-(Azetidin-1-yl)-N-(2,6-dipropoxybenzene-1-sulfonyl)-4-fluoro-1-benzofuran-2-carboxamide,
6-(Azetidin-1-yl)-4-fluoro-N-[2-methyl-2,3-dihydro-1-benzofuran-7-sulfonyl]-1-benzofuran-2-carboxamide,
6-(Azetidin-1-yl)-N-([1,1′-biphenyl]-2-sulfonyl)-4-fluoro-1-benzofuran-2-carboxamide,
Sodium {[6-(azetidin-1-yl)-4-fluoro-1-benzofuran-2-yl]carbonyl}([biphenyl]-2-ylsulfonyl)azanide,
6-(Azetidin-1-yl)-N-(2,2-dimethyl-2,3-dihydro-1-benzofuran-7-sulfonyl)-4-fluoro-1-benzofuran-2-carboxamide,
6-(Azetidin-1-yl)-4-fluoro-N-(2-iodobenzene-1-sulfonyl)-1-benzofuran-2-carboxamide,
6-(Azetidin-1-yl)-N-[2-ethoxy-6-(propan-2-yl)benzene-1-sulfonyl]-4-fluoro-1-benzofuran-2-carboxamide,
6-(Azetidin-1-yl)-N-(2-cyclobutyl-6-fluorobenzene-1-sulfonyl)-4-fluoro-1-benzofuran-2-carboxamide,
6-(Azetidin-1-yl)-N-(2-ethoxy-6-methylbenzene-1-sulfonyl)-4-fluoro-1-benzofuran-2-carboxamide,
6-(Azetidin-1-yl)-N-([1,1′-biphenyl]-2-sulfonyl)-1-benzofuran-2-carboxamide,
6-(Azetidin-1-yl)-N-(3-chloro-6-methoxy-2-methylbenzene-1-sulfonyl)-4-fluoro-1-benzofuran-2-carboxamide,
6-(Azetidin-1-yl)-4-fluoro-N-{2-[(propan-2-yl)oxy]-6-(trifluoromethyl)benzene-1-sulfonyl}-1-benzofuran-2-carboxamide,
6-(Azetidin-1-yl)-N-{2-chloro-6-[(propan-2-yl)oxy]benzene-1-sulfonyl}-4-fluoro-1-benzofuran-2-carboxamide,
6-(Azetidin-1-yl)-N-[2,5-di(propan-2-yl)benzene-1-sulfonyl]-4-fluoro-1-benzofuran-2-carboxamide,
6-(Azetidin-1-yl)-4-fluoro-N-(2-propoxy-6-propylbenzene-1-sulfonyl)-1-benzofuran-2-carboxamide,
6-(Azetidin-1-yl)-4-fluoro-N-[2-methyl-5-(propan-2-yl)benzene-1-sulfonyl]-1-benzofuran-2-carboxamide,
6-(Azetidin-1-yl)-N-[2-(cyclopropylmethoxy)-6-ethylbenzene-1-sulfonyl]-4-fluoro-1-benzofuran-2-carboxamide,
6-(Azetidin-1-yl)-N-(5-ethyl-2-methylbenzene-1-sulfonyl)-4-fluoro-1-benzofuran-2-carboxamide,
6-(Azetidin-1-yl)-N-[2-(difluoromethoxy)-4-methylbenzene-1-sulfonyl]-4-fluoro-1-benzofuran-2-carboxamide,
6-(Azetidin-1-yl)-N-(4-ethyl-2-methylthiophene-3-sulfonyl)-4-fluoro-1-benzofuran-2-carboxamide,
6-(Azetidin-1-yl)-4-fluoro-N-[2-(3-methyl-1,2,4-oxadiazol-5-yl)benzene-1-sulfonyl]-1-benzofuran-2-carboxamide,
6-(Azetidin-1-yl)-N-[4-bromo-2-(propan-2-yl)benzene-1-sulfonyl]-4-fluoro-1-benzofuran-2-carboxamide,
6-(Azetidin-1-yl)-N-{2-bromo-6-[(propan-2-yl)oxy]benzene-1-sulfonyl}-4-fluoro-1-benzofuran-2-carboxamide,
6-(Azetidin-1-yl)-4-fluoro-N-(3-propylbenzene-1-sulfonyl)-1-benzofuran-2-carboxamide,
6-(Azetidin-1-yl)-4-fluoro-N-(1-methyl-1H-indole-7-sulfonyl)-1-benzofuran-2-carboxamide,
6-(Azetidin-1-yl)-4-fluoro-N-{2-fluoro-6-[(propan-2-yl)amino]benzene-1-sulfonyl}-1-benzofuran-2-carboxamide,
6-(Azetidin-1-yl)-4-fluoro-N-(2-fluoro-5-methylbenzene-1-sulfonyl)-1-benzofuran-2-carboxamide,
6-(Azetidin-1-yl)-N-[2-(3-ethyl-1,2,4-oxadiazol-5-yl)benzene-1-sulfonyl]-4-fluoro-1-benzofuran-2-carboxamide,
6-(Azetidin-1-yl)-N-[2-(benzenesulfonyl)benzene-1-sulfonyl]-4-fluoro-1-benzofuran-2-carboxamide,
6-(azetidin-1-yl)-N-[2-(ethylamino)benzene-1-sulfonyl]-4-fluoro-1-benzofuran-2-carboxamide,
6-(Azetidin-1-yl)-N-{2-[ethyl(methyl)carbamoyl]benzene-1-sulfonyl}-4-fluoro-1-benzofuran-2-carboxamide,
6-(Azetidin-1-yl)-4-fluoro-N-[3-(methoxymethyl)benzene-1-sulfonyl]-1-benzofuran-2-carboxamide,
6-(Azetidin-1-yl)-N-{5-chloro-2-[(propan-2-yl)amino]benzene-1-sulfonyl}-4-fluoro-1-benzofuran-2-carboxamide,
6-(Azetidin-1-yl)-N-[2-(cyclopropylamino)-6-fluorobenzene-1-sulfonyl]-4-fluoro-1-benzofuran-2-carboxamide,
6-(Azetidin-1-yl)-N-[2-(dimethylcarbamoyl)benzene-1-sulfonyl]-4-fluoro-1-benzofuran-2-carboxamide,
6-(Azetidin-1-yl)-4-fluoro-N-[2-(3,3,3-trifluoropropyl)benzene-1-sulfonyl]-1-benzofuran-2-carboxamide,
6-(Azetidin-1-yl)-4-fluoro-N-[2-methyl-6-(propan-2-yl)benzene-1-sulfonyl]-1-benzofuran-2-carboxamide,
6-(Azetidin-1-yl)-N-[2-chloro-5-(ethoxymethyl)benzene-1-sulfonyl]-4-fluoro-1-benzofuran-2-carboxamide,
6-(Azetidin-1-yl)-4-fluoro-N-{2-[(1E)-3,3,3-trifluoroprop-1-en-1-yl]benzene-1-sulfonyl}-1-benzofuran-2-carboxamide,
6-(Azetidin-1-yl)-4-fluoro-N-(quinoxaline-5-sulfonyl)-1-benzofuran-2-carboxamide,
Methyl (5-{[6-(azetidin-1-yl)-4-fluoro-1-benzofuran-2-carbonyl]sulfamoyl}naphthalen-2-yl)acetate,
6-(Azetidin-1-yl)-4-fluoro-N-[2-propyl-6-(trifluoromethyl)benzene-1-sulfonyl]-1-benzofuran-2-carboxamide,
6-(Azetidin-1-yl)-N-[2-chloro-5-(2-methoxyethoxy)benzene-1-sulfonyl]-4-fluoro-1-benzofuran-2-carboxamide,
6-(Azetidin-1-yl)-N-(2-chloro-5-methylpyridine-3-sulfonyl)-4-fluoro-1-benzofuran-2-carboxamide,
6-(Azetidin-1-yl)-N-(2-cyclopentyl-6-methylbenzene-1-sulfonyl)-4-fluoro-1-benzofuran-2-carboxamide,
2-{[6-(Azetidin-1-yl)-4-fluoro-1-benzofuran-2-carbonyl]sulfamoyl}phenyl
methyl(phenyl)carbamate,
6-(Azetidin-1-yl)-4-fluoro-N-[5-(hydroxymethyl)-2-(trifluoromethoxy)benzene-1-sulfonyl]-1-benzofuran-2-carboxamide,
6-(Azetidin-1-yl)-4-fluoro-N-(2-hydroxy-6-methylbenzene-1-sulfonyl)-1-benzofuran-2-carboxamide,
6-(Azetidin-1-yl)-4-fluoro-N-(5-methoxynaphthalene-1-sulfonyl)-1-benzofuran-2-carboxamide,
6-(Azetidin-1-yl)-N-[5-(dimethylamino)naphthalene-1-sulfonyl]-4-fluoro-1-benzofuran-2-carboxamide,
6-(Azetidin-1-yl)-4-fluoro-N-(3-methoxypyridine-2-sulfonyl)-1-benzofuran-2-carboxamide,
6-(Azetidin-1-yl)-N-[4-(2-cyanoethyl)benzene-1-sulfonyl]-4-fluoro-1-benzofuran-2-carboxamide,
6-(Azetidin-1-yl)-N-(3-bromopyridine-2-sulfonyl)-4-fluoro-1-benzofuran-2-carboxamide,
6-(Azetidin-1-yl)-4-fluoro-N-{2-[(3-methyl-1-phenyl-1H-pyrazol-5-yl)amino]benzene-1-sulfonyl}-1-benzofuran-2-carboxamide,
6-(Azetidin-1-yl)-4-fluoro-N-(3-hydroxynaphthalene-1-sulfonyl)-1-benzofuran-2-carboxamide,
6-(Azetidin-1-yl)-N-[2-chloro-5-(dimethylsulfamoyl)benzene-1-sulfonyl]-4-fluoro-1-benzofuran-2-carboxamide,
6-(Azetidin-1-yl)-4-fluoro-N-[2-(morpholine-4-carbonyl)benzene-1-sulfonyl]-1-benzofuran-2-carboxamide,
Methyl (2E)-3-(2-{[6-(azetidin-1-yl)-4-fluoro-1-benzofuran-2-carbonyl]sulfamoyl}phenyl)prop-2-enoate,
6-(Azetidin-1-yl)-N-(3-ethoxybenzene-1-sulfonyl)-4-fluoro-1-benzofuran-2-carboxamide,
2-{[6-(Azetidin-1-yl)-4-fluoro-1-benzofuran-2-carbonyl]sulfamoyl}phenyl diethylcarbamate,
6-(Azetidin-1-yl)-N-[2-(ethanesulfonyl)benzene-1-sulfonyl]-4-fluoro-1-benzofuran-2-carboxamide,
6-(azetidin-1-yl)-N-(5-tert-butyl-2-ethoxybenzene-1-sulfonyl)-4-fluoro-1-benzofuran-2-carboxamide,
6-(Azetidin-1-yl)-4-fluoro-N-[3-(propan-2-yl)pyridine-2-sulfonyl]-1-benzofuran-2-carboxamide,
6-(Azetidin-1-yl)-N-(2,2-dimethyl-3,4-dihydro-2H-1-benzopyran-8-sulfonyl)-4-fluoro-1-benzofuran-2-carboxamide,
6-(Azetidin-1-yl)-N-[2-(2,2-difluoroethoxy)-6-ethoxybenzene-1-sulfonyl]-4-fluoro-1-benzofuran-2-carboxamide,
6-(Azetidin-1-yl)-N-[5-bromo-2-(trifluoromethoxy)benzene-1-sulfonyl]-4-fluoro-1-benzofuran-2-carboxamide,
6-(Azetidin-1-yl)-N-(2-tert-butoxy-6-propoxybenzene-1-sulfonyl)-4-fluoro-1-benzofuran-2-carboxamide,
6-(Azetidin-1-yl)-N-{2-ethyl-6-[(propan-2-yl)oxy]benzene-1-sulfonyl}-4-fluoro-1-benzofuran-2-carboxamide,
6-(Azetidin-1-yl)-N-[2-(benzyloxy)-6-ethylbenzene-1-sulfonyl]-4-fluoro-1-benzofuran-2-carboxamide,
6-(Azetidin-1-yl)-4-fluoro-N-{2-methyl-6-[(propan-2-yl)oxy]benzene-1-sulfonyl}-1-benzofuran-2-carboxamide,
6-(azetidin-1-yl)-4-fluoro-N-(4-methylnaphthalene-1-sulfonyl)-1-benzofuran-2-carboxamide,
6-(Azetidin-1-yl)-N-(2,6-difluoro-3-methylbenzene-1-sulfonyl)-4-fluoro-1-benzofuran-2-carboxamide,
6-(Azetidin-1-yl)-4-fluoro-N-(5-propanamidonaphthalene-1-sulfonyl)-1-benzofuran-2-carboxamide,
Ethyl 8-{[6-(azetidin-1-yl)-4-fluoro-1-benzofuran-2-carbonyl]sulfamoyl}imidazo[1,2-a]pyridine-2-carboxylate,
6-(Azetidin-1-yl)-4-fluoro-N-[3-methyl-1-(propan-2-yl)-1H-pyrazole-4-sulfonyl]-1-benzofuran-2-carboxamide,
6-(Azetidin-1-yl)-N-{2-[(2,2-difluoroethyl)amino]-5-(trifluoromethyl)benzene-1-sulfonyl}-4-fluoro-1-benzofuran-2-carboxamide,
[3-sulfamoyl-4-(trifluoromethoxy)phenyl]methyl 6-(azetidin-1-yl)-4-fluoro-1-benzofuran-2-carboxylate,
6-(Azetidin-1-yl)-4-fluoro-N-(2-methylpyridine-3-sulfonyl)-1-benzofuran-2-carboxamide,
2-{[6-(Azetidin-1-yl)-4-fluoro-1-benzofuran-2-carbonyl]sulfamoyl}-N,N-dimethylpyridine-3-carboxamide,
6-(Azetidin-1-yl)-N-[2-(5,6-dihydro-1,4,2-dioxazin-3-yl)benzene-1-sulfonyl]-4-fluoro-1-benzofuran-2-carboxamide,
6-(Azetidin-1-yl)-N-[2-chloro-5-(dimethylamino)benzene-1-sulfonyl]-4-fluoro-1-benzofuran-2-carboxamide,
4-Sulfamoylnaphthalen-2-yl 6-(azetidin-1-yl)-4-fluoro-1-benzofuran-2-carboxylate,
N-(5-Acetyl-2-chlorobenzene-1-sulfonyl)-6-(azetidin-1-yl)-4-fluoro-1-benzofuran-2-carboxamide,
6-(Azetidin-1-yl)-N-[2-chloro-5-(1-hydroxyethyl)benzene-1-sulfonyl]-4-fluoro-1-benzofuran-2-carboxamide (rac),
Propan-2-yl 2-{[6-(azetidin-1-yl)-4-fluoro-1-benzofuran-2-carbonyl]sulfamoyl}benzoate,
Methyl 3-(2-{[6-(azetidin-1-yl)-4-fluoro-1-benzofuran-2-carbonyl]sulfamoyl}phenyl)propanoate,
6-(Azetidin-1-yl)-N-[2-chloro-5-(trifluoromethyl)pyridine-3-sulfonyl]-4-fluoro-1-benzofuran-2-carboxamide,
N-(2-amino-5-chloro-4,6-dimethylpyridine-3-sulfonyl)-6-(azetidin-1-yl)-4-fluoro-1-benzofuran-2-carboxamide,
Methyl {[1-{2-[(2-ethoxybenzene-1-sulfonyl)carbamoyl]-4-fluoro-1-benzofuran-6-yl}azetidin-2-yl]methyl}carbamate (rac),
6-(Azetidin-1-yl)-4-fluoro-N-(4-hydroxynaphthalene-1-sulfonyl)-1-benzofuran-2-carboxamide,
N-(2-Ethoxybenzene-1-sulfonyl)-6-(3-fluoroazetidin-1-yl)-1-benzofuran-2-carboxamide,
4-Fluoro-6-(3-fluoroazetidin-1-yl)-N-(5,6,7,8-tetrahydronaphthalene-1-sulfonyl)-1-benzofuran-2-carboxamide,
N-(2,3-dihydro-1,4-benzodioxine-5-sulfonyl)-4-fluoro-6-(3-fluoroazetidin-1-yl)-1-benzofuran-2-carboxamide,
4-Fluoro-6-(3-fluoroazetidin-1-yl)-N-(1,2,3,4-tetrahydroquinoline-8-sulfonyl)-1-benzofuran-2-carboxamide,
4-Fluoro-6-(3-fluoroazetidin-1-yl)-N-(quinoline-4-sulfonyl)-1-benzofuran-2-carboxamide,
4-Fluoro-6-(3-fluoroazetidin-1-yl)-N-(2-methyl-1,2,3,4-tetrahydroisoquinoline-5-sulfonyl)-1-benzofuran-2-carboxamide,
N-(5-tert-butyl-2-ethoxybenzene-1-sulfonyl)-4-fluoro-6-(3-fluoroazetidin-1-yl)-1-benzofuran-2-carboxamide,
N-[2-(dichloromethyl)benzene-1-sulfonyl]-4-fluoro-6-(3-fluoroazetidin-1-yl)-1-benzofuran-2-carboxamide,
4-Fluoro-6-(3-fluoroazetidin-1-yl)-N-[4-(2-methyl-1,3-thiazol-4-yl)benzene-1-sulfonyl]-1-benzofuran-2-carboxamide,
N-(2-Ethoxybenzene-1-sulfonyl)-6-(3-fluoro-3-methylazetidin-1-yl)-1-benzofuran-2-carboxamide,
N-(2-Ethoxybenzene-1-sulfonyl)-6-[3-(trifluoromethyl)azetidin-1-yl]-1-benzofuran-2-carboxamide,
6-(3,3-Difluoroazetidin-1-yl)-N-(2-ethoxybenzene-1-sulfonyl)-1-benzofuran-2-carboxamide,
N-([1,1′-biphenyl]-2-sulfonyl)-6-(3,3-difluoroazetidin-1-yl)-1-benzofuran-2-carboxamide,
N-(2-ethoxybenzene-1-sulfonyl)-6-(3-phenylazetidin-1-yl)-1-benzofuran-2-carboxamide,
6-(3-Ethoxyazetidin-1-yl)-N-(2-ethoxybenzene-1-sulfonyl)-1-benzofuran-2-carboxamide,
6-(5-Azaspiro[2.3]hexan-5-yl)-N-(2-ethoxybenzene-1-sulfonyl)-1-benzofuran-2-carboxamide,
6-(2-Azaspiro[3.3]heptan-2-yl)-N-(2-ethoxybenzene-1-sulfonyl)-1-benzofuran-2-carboxamide,
6-(3,3-dimethylazetidin-1-yl)-N-(2-ethoxybenzene-1-sulfonyl)-1-benzofuran-2-carboxamide,
N-(2-ethoxybenzene-1-sulfonyl)-6-(2-oxa-6-azaspiro[3.3]heptan-6-yl)-1-benzofuran-2-carboxamide,
Sodium [6-(azetidin-1-yl)-4-fluoro-1-benzofuran-2-carbonyl](2-ethoxybenzene-1-sulfonyl)azanide,
6-(Azetidin-1-yl)-N-(5-tert-butyl-2-methoxybenzene-1-sulfonyl)-4-fluoro-1-benzofuran-2-carboxamide,
6-(Azetidin-1-yl)-N-[2-ethoxy-5-(propan-2-yl)benzene-1-sulfonyl]-4-fluoro-1-benzofuran-2-carboxamide,
6-(Azetidin-1-yl)-4-fluoro-N-{2-[(propan-2-yl)oxy]benzene-1-sulfonyl}-1-benzofuran-2-carboxamide,
6-(Azetidin-1-yl)-4-fluoro-N-(2-methylquinoline-8-sulfonyl)-1-benzofuran-2-carboxamide,
6-(Azetidin-1-yl)-N-[2-(cyclopropyloxy)benzene-1-sulfonyl]-4-fluoro-1-benzofuran-2-carboxamide,
6-(Azetidin-1-yl)-N-(2-chloroquinoline-8-sulfonyl)-4-fluoro-1-benzofuran-2-carboxamide,
6-(Azetidin-1-yl)-4-fluoro-N-(2-propoxybenzene-1-sulfonyl)-1-benzofuran-2-carboxamide,
6-(Azetidin-1-yl)-4-fluoro-N-{5-methyl-2-[(propan-2-yl)oxy]benzene-1-sulfonyl}-1-benzofuran-2-carboxamide,
6-(Azetidin-1-yl)-N-(5-bromo-2-ethoxybenzene-1-sulfonyl)-4-fluoro-1-benzofuran-2-carboxamide,
6-(azetidin-1-yl)-N-(2-ethyl-6-methoxybenzene-1-sulfonyl)-4-fluoro-1-benzofuran-2-carboxamide,
6-(Azetidin-1-yl)-4-fluoro-N-[2-methoxy-5-(morpholin-4-yl)benzene-1-sulfonyl]-1-benzofuran-2-carboxamide,
6-(Azetidin-1-yl)-4-fluoro-N-[2-(trifluoromethoxy)benzene-1-sulfonyl]-1-benzofuran-2-carboxamide,
6-(Azetidin-1-yl)-N-(5-bromo-2-methoxybenzene-1-sulfonyl)-4-fluoro-1-benzofuran-2-carboxamide,
6-(Azetidin-1-yl)-4-fluoro-N-(2,2,3,3-tetrafluoro-2,3-dihydro-1,4-benzodioxine-5-sulfonyl)-1-benzofuran-2-carboxamide,
6-(Azetidin-1-yl)-N-(1,1-dioxo-1,2,3,4-tetrahydro-1-benzothiopyran-8-sulfonyl)-4-fluoro-1-benzofuran-2-carboxamide,
6-(Azetidin-1-yl)-N-(5-chloro-2,2-difluoro-2H-1,3-benzodioxole-4-sulfonyl)-4-fluoro-1-benzofuran-2-carboxamide,
6-(Azetidin-1-yl)-N-(1-ethyl-1H-pyrazolo[3,4-b]pyridine-3-sulfonyl)-4-fluoro-1-benzofuran-2-carboxamide,
6-(Azetidin-1-yl)-4-fluoro-N-[1-methyl-6-(trifluoromethyl)-1H-pyrazolo[3,4-b]pyridine-3-sulfonyl]-1-benzofuran-2-carboxamide,
6-(Azetidin-1-yl)-N-(1,6-dimethyl-1H-pyrazolo[3,4-b]pyridine-3-sulfonyl)-4-fluoro-1-benzofuran-2-carboxamide,
6-(Azetidin-1-yl)-N-(1,4-dimethyl-1H-pyrazolo[3,4-b]pyridine-3-sulfonyl)-4-fluoro-1-benzofuran-2-carboxamide,
6-(Azetidin-1-yl)-N-(2-chloro-6-propylimidazo[1,2-b]pyridazine-3-sulfonyl)-4-fluoro-1-benzofuran-2-carboxamide,
6-(Azetidin-1-yl)-N-[2-(ethylsulfanyl)imidazo[1,2-a]pyridine-3-sulfonyl]-4-fluoro-1-benzofuran-2-carboxamide,
6-(Azetidin-1-yl)-4-fluoro-N-[2-(methylsulfanyl)imidazo[1,2-a]pyridine-3-sulfonyl]-1-benzofuran-2-carboxamide,
N-(5-tert-butyl-2-methoxybenzene-1-sulfonyl)-4-fluoro-6-(3-fluoroazetidin-1-yl)-1-benzofuran-2-carboxamide,
N-[2-ethoxy-5-(propan-2-yl)benzene-1-sulfonyl]-4-fluoro-6-(3-fluoroazetidin-1-yl)-1-benzofuran-2-carboxamide,
4-Fluoro-6-(3-fluoroazetidin-1-yl)-N-(2-methylquinoline-8-sulfonyl)-1-benzofuran-2-carboxamide,
N-(2-ethoxy-5-ethylbenzene-1-sulfonyl)-4-fluoro-6-(3-fluoroazetidin-1-yl)-1-benzofuran-2-carboxamide,
N-(2-Chloroquinoline-8-sulfonyl)-4-fluoro-6-(3-fluoroazetidin-1-yl)-1-benzofuran-2-carboxamide,
4-Fluoro-6-(3-fluoroazetidin-1-yl)-N-{2-[(propan-2-yl)oxy]benzene-1-sulfonyl}-1-benzofuran-2-carboxamide,
6-(Azetidin-1-yl)-4-fluoro-N-(2-methoxy-4-methylbenzene-1-sulfonyl)-1-benzofuran-2-carboxamide,
4-Fluoro-6-(3-fluoroazetidin-1-yl)-N-{5-methyl-2-[(propan-2-yl)oxy]benzene-1-sulfonyl}-1-benzofuran-2-carboxamide,
4-Fluoro-6-(3-fluoroazetidin-1-yl)-N-(2-methoxy-4-methylbenzene-1-sulfonyl)-1-benzofuran-2-carboxamide,
4-Fluoro-6-(3-fluoroazetidin-1-yl)-N-(2,2,3,3-tetrafluoro-2,3-dihydro-1,4-benzodioxine-5-sulfonyl)-1-benzofuran-2-carboxamide,
6-[2-(Difluoromethyl)azetidin-1-yl]-4-fluoro-N-(2-methylquinoline-8-sulfonyl)-1-benzofuran-2-carboxamide (rac),
4-Fluoro-6-[2-(1-hydroxy-1-methyl-ethyl)azetidin-1-yl]-N-[(2-methyl-8-quinolyl)sulfonyl]benzofuran-2-carboxamide (rac),
N-(2-ethoxybenzene-1-sulfonyl)-4-fluoro-6-[2-(2-hydroxypropan-2-yl)azetidin-1-yl]-1-benzofuran-2-carboxamide (rac),
6-[2-(Difluoromethyl)azetidin-1-yl]-N-(2-ethoxybenzene-1-sulfonyl)-4-fluoro-1-benzofuran-2-carboxamide (rac),
N-{2,6-difluoro-4-[methyl(propan-2-yl)amino]benzene-1-sulfonyl}-4-fluoro-6-(3-fluoroazetidin-1-yl)-1-benzofuran-2-carboxamide,
N-{2,4-difluoro-6-[methyl(propan-2-yl)amino]benzene-1-sulfonyl}-4-fluoro-6-(3-fluoroazetidin-1-yl)-1-benzofuran-2-carboxamide,
N-{2,3-difluoro-4-[methyl(propan-2-yl)amino]benzene-1-sulfonyl}-4-fluoro-6-(3-fluoroazetidin-1-yl)-1-benzofuran-2-carboxamide,
6-(Azetidin-1-yl)-N-[2-(ethanesulfonyl)imidazo[1,2-a]pyridine-3-sulfonyl]-4-fluoro-1-benzofuran-2-carboxamide,
6-(Azetidin-1-yl)-N-(6,8-dichloroimidazo[1,2-a]pyridine-3-sulfonyl)-4-fluoro-1-benzofuran-2-carboxamide,
1-12-[(2-Ethoxybenzene-1-sulfonyl)carbamoyl]-4-fluoro-1-benzofuran-6-yl}azetidine-2-carboxamide (rac),
6-(azetidin-1-yl)-N-{5-bromo-2-[(propan-2-yl)oxy]benzene-1-sulfonyl}-4-fluoro-1-benzofuran-2-carboxamide,
6-(Azetidin-1-yl)-N-(2-ethylquinoline-8-sulfonyl)-4-fluoro-1-benzofuran-2-carboxamide,
6-(Azetidin-1-yl)-N-(2,3-dihydro-1,4-benzodioxine-5-sulfonyl)-4-fluoro-1-benzofuran-2-carboxamide,
6-(Azetidin-1-yl)-4-fluoro-N-(7-methylquinoline-8-sulfonyl)-1-benzofuran-2-carboxamide,
6-(Azetidin-1-yl)-N-(4-bromo-2-methoxybenzene-1-sulfonyl)-4-fluoro-1-benzofuran-2-carboxamide,
6-(Azetidin-1-yl)-4-fluoro-N-(6-methylquinoline-8-sulfonyl)-1-benzofuran-2-carboxamide,
6-(Azetidin-1-yl)-4-fluoro-N-(1H-indole-3-sulfonyl)-1-benzofuran-2-carboxamide,
6-(Azetidin-1-yl)-N-(5-bromo-2,3-dihydro-1-benzofuran-7-sulfonyl)-4-fluoro-1-benzofuran-2-carboxamide,
6-(Azetidin-1-yl)-N-(1-benzothiophene-3-sulfonyl)-4-fluoro-1-benzofuran-2-carboxamide,
6-(Azetidin-1-yl)-4-fluoro-N-(2-methyl-2H-benzotriazole-4-sulfonyl)-1-benzofuran-2-carboxamide,
6-(Azetidin-1-yl)-4-fluoro-N-(4-methoxy-5,6,7,8-tetrahydronaphthalene-1-sulfonyl)-1-benzofuran-2-carboxamide,
6-(Azetidin-1-yl)-N-(2,1,3-benzothiadiazole-4-sulfonyl)-4-fluoro-1-benzofuran-2-carboxamide,
6-(Azetidin-1-yl)-4-fluoro-N-(5-methoxy-1,2,3-benzothiadiazole-4-sulfonyl)-1-benzofuran-2-carboxamide,
6-(Azetidin-1-yl)-N-(3,4-dichloro-2-methoxybenzene-1-sulfonyl)-4-fluoro-1-benzofuran-2-carboxamide,
6-(Azetidin-1-yl)-N-(6,7-dihydro-5H-pyrazolo[5,1-b][1,3]oxazine-3-sulfonyl)-4-fluoro-1-benzofuran-2-carboxamide,
N-(4-Aacetamidonaphthalene-1-sulfonyl)-6-(azetidin-1-yl)-4-fluoro-1-benzofuran-2-carboxamide,
6-(Azetidin-1-yl)-4-fluoro-N-(imidazo[1,2-a]pyridine-3-sulfonyl)-1-benzofuran-2-carboxamide,
6-(Azetidin-1-yl)-4-fluoro-N-(isoquinoline-5-sulfonyl)-1-benzofuran-2-carboxamide,
6-(Azetidin-1-yl)-4-fluoro-N-[5-(morpholin-4-yl)naphthalene-1-sulfonyl]-1-benzofuran-2-carboxamide,
6-(Azetidin-1-yl)-N-(5-tert-butyl-2,3-dimethylbenzene-1-sulfonyl)-4-fluoro-1-benzofuran-2-carboxamide,
6-(Azetidin-1-yl)-N-(3,3-dimethyl-2,3-dihydro-1-benzofuran-5-sulfonyl)-4-fluoro-1-benzofuran-2-carboxamide,
6-(Azetidin-1-yl)-4-fluoro-N-(8-methylisoquinoline-5-sulfonyl)-1-benzofuran-2-carboxamide,
Methyl (5-{[6-(azetidin-1-yl)-4-fluoro-1-benzofuran-2-carbonyl]sulfamoyl}naphthalen-1-yl)carbamate,
6-(Azetidin-1-yl)-4-fluoro-N-(4-methylisoquinoline-5-sulfonyl)-1-benzofuran-2-carboxamide,
6-(Azetidin-1-yl)-N-[5-(3,5-dimethyl-1,2-oxazol-4-yl)-2-methoxybenzene-1-sulfonyl]-4-fluoro-1-benzofuran-2-carboxamide,
6-(Azetidin-1-yl)-N-[5-(1,3-dimethyl-1H-pyrazol-4-yl)-2-methoxybenzene-1-sulfonyl]-4-fluoro-1-benzofuran-2-carboxamide,
6-(azetidin-1-yl)-N-[5-(3,6-dihydro-2H-pyran-4-yl)-2-methoxybenzene-1-sulfonyl]-4-fluoro-1-benzofuran-2-carboxamide,
tert-butyl 4-(3-{[6-(azetidin-1-yl)-4-fluoro-1-benzofuran-2-carbonyl]sulfamoyl}-4-methoxyphenyl)-3,6-dihydropyridine-1(2H)-carboxylate,
6-(Azetidin-1-yl)-4-fluoro-N-(3′-fluoro-4-methoxy[1,1′-biphenyl]-3-sulfonyl)-1-benzofuran-2-carboxamide,
6-(Azetidin-1-yl)-4-fluoro-N-[2-methoxy-5-(1-methyl-1H-pyrazol-4-yl)benzene-1-sulfonyl]-1-benzofuran-2-carboxamide,
6-(Azetidin-1-yl)-4-fluoro-N-[2-methoxy-5-(pyridin-3-yl)benzene-1-sulfonyl]-1-benzofuran-2-carboxamide,
6-(Azetidin-1-yl)-4-fluoro-N-[2-methoxy-5-(2-phenylpyrrolidin-1-yl)benzene-1-sulfonyl]-1-benzofuran-2-carboxamide (rac),
6-(Azetidin-1-yl)-4-fluoro-N-[2-methoxy-5-(4-methylpiperazin-1-yl)benzene-1-sulfonyl]-1-benzofuran-2-carboxamide,
6-(Azetidin-1-yl)-4-fluoro-N-[2-methoxy-5-(1-methyl-1,2,3,6-tetrahydropyridin-4-yl)benzene-1-sulfonyl]-1-benzofuran-2-carboxamide,
6-(Azetidin-1-yl)-4-fluoro-N-(2,2,5,6-tetramethyl-2,3-dihydro-1-benzofuran-7-sulfonyl)-1-benzofuran-2-carboxamide,
6-(Azetidin-1-yl)-4-fluoro-N-[2-methoxy-5-(prop-1-en-2-yl)benzene-1-sulfonyl]-1-benzofuran-2-carboxamide,
6-(azetidin-1-yl)-N-(2-ethoxy-3-fluoro-phenyl)sulfonyl-4-fluoro-benzofuran-2-carboxamide,
6-(Azetidin-1-yl)-N-[(5-sec-butyl-2-ethoxyphenyl)sulfonyl]-4-fluoro-1-benzofuran-2-carboxamide (rac),
6-(Azetidin-1-yl)-N-(2-ethoxy-4-fluoro-phenyl)sulfonyl-4-fluoro-benzofuran-2-carboxamide,
6-(azetidin-1-yl)-N-[2-ethoxy-5-(trifluoromethyl)phenyl]sulfonyl-4-fluoro-benzofuran-2-carboxamide,
6-(Azetidin-1-yl)-N-(2-ethoxy-5-fluoro-phenyl)sulfonyl-4-fluoro-benzofuran-2-carboxamide,
6-(Azetidin-1-yl)-N-(2-ethoxy-5-phenoxy-phenyl)sulfonyl-4-fluoro-benzofuran-2-carboxamide,
6-(Azetidin-1-yl)-N-[2-ethoxy-5-(trifluoromethoxy)phenyl]sulfonyl-4-fluoro-benzofuran-2-carboxamide,
6-(azetidin-1-yl)-N-(2-benzyloxy-5-isopropyl-phenyl)sulfonyl-4-fluoro-benzofuran-2-carboxamide,
6-(azetidin-1-yl)-N-[2-(cyclopropylmethoxy)-5-isopropyl-phenyl]sulfonyl-4-fluoro-benzofuran-2-carboxamide,
6-(azetidin-1-yl)-4-fluoro-N-[5-isopropyl-2-(2,2,2-trifluoroethoxy)phenyl]sulfonyl-benzofuran-2-carboxamide,
N-(benzenesulfonyl)-6-(2,2-dimethylazetidin-1-yl)-4-fluoro-benzofuran-2-carboxamide,
6-(2,2-Dimethylazetidin-1-yl)-4-fluoro-N-(2-phenylphenyl)sulfonyl-benzofuran-2-carboxamide,
6-(2,2-Dimethylazetidin-1-yl)-4-fluoro-N-[(2-methyl-8-quinolyl)sulfonyl]benzofuran-2-carboxamide,
6-(2,2-Dimethylazetidin-1-yl)-N-(2-ethoxyphenyl)sulfonyl-4-fluoro-benzofuran-2-carboxamide,
N-(Benzenesulfonyl)-4-fluoro-6-(2-methylazetidin-1-yl)benzofuran-2-carboxamide (rac),
4-Fluoro-6-(2-methylazetidin-1-yl)-N-[(2-methyl-8-quinolyl)sulfonyl]benzofuran-2-carboxamide (rac),
4-Fluoro-6-(2-methylazetidin-1-yl)-N-(2-phenylphenyl)sulfonyl-benzofuran-2-carboxamide (rac),
N-(2-Ethoxyphenyl)sulfonyl-4-fluoro-6-(2-methylazetidin-1-yl)benzofuran-2-carboxamide (rac),
6-(Azetidin-1-yl)-N-[(5-(tert-butyl)-2-cyclopropoxyphenyl)sulfonyl]-4-fluorobenzofuran-2-carboxamide,
6-(Azetidin-1-yl)-N-[(2-(benzyloxy)-5-(tert-butyl)phenyl]sulfonyl)-4-fluorobenzofuran-2-carboxamide,
6-(Azetidin-1-yl)-N-[(5-(tert-butyl)-2-(cyclopropylmethoxy)phenyl)sulfonyl]-4-fluorobenzofuran-2-carboxamide,
6-(Azetidin-1-yl)-N-[(5-(tert-butyl)-2-(2,2,2-trifluoroethoxy)phenyl)sulfonyl]-4-fluorobenzofuran-2-carboxamide,
6-(Azetidin-1-yl)-N-[(5-(tert-butyl)-2-cyclobutoxyphenyl)sulfonyl]-4-fluorobenzofuran-2-carboxamide,
6-(Azetidin-1-yl)-N-[(5-(tert-butyl)-2-isopropoxyphenyl)sulfonyl]-4-fluorobenzofuran-2-carboxamide,
6-(Azetidin-1-yl)-N-[(2-cyclobutoxy-5-isopropylphenyl)sulfonyl]-4-fluorobenzofuran-2-carboxamide,
6-(Azetidin-1-yl)-N-((2-((2,2-difluorocyclopropyl)methoxy)-5-isopropylphenyl)sulfonyl)-4-fluorobenzofuran-2-carboxamide (rac),
6-(Azetidin-1-yl)-N-((2-ethoxy-4,5-difluorophenyl)sulfonyl)-4-fluorobenzofuran-2-carboxamide,
6-(Azetidin-1-yl)-4-fluoro-N-[(2-methoxy-5-(methoxymethyl)phenyl)sulfonyl]benzofuran-2-carboxamide,
6-(Azetidin-1-yl)-N-[(5-cyclopropyl-2-methoxyphenyl)sulfonyl]-4-fluorobenzofuran-2-carboxamide,
6-(Azetidin-1-yl)-N-((5-cyclopropyl-2-ethoxyphenyl)sulfonyl)-4-fluorobenzofuran-2-carboxamide,
6-(Azetidin-1-yl)-4-fluoro-N-[(2-methoxy-5-(2-oxopropyl)phenyl)sulfonyl]benzofuran-2-carboxamide and
tert-butyl 4-((2-(N-(6-(azetidin-1-yl)-4-fluorobenzofuran-2-carbonyl)sulfamoyl)-4-(tert-butyl)phenoxy)methyl)piperidine-1-carboxylate.

7. A method of preparing a compound of general formula (I) according to claim 1, wherein R⁵ is a hydrogen atom and R¹, R², R³, R⁴, R⁶, R^{a} and R^{b} are as defined for the compounds of general formula (I) according to claim 1, or a tautomer, or an N-oxide, or a salt thereof, or a salt of a tautomer, or a salt of an N-oxide, or a mixture of same, said method comprising the reaction of an intermediate compound of formula (IIb) 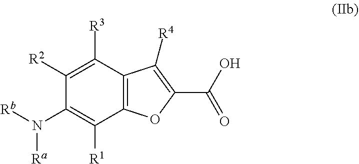
with a sulfonamide of general formula H₂N—SO₂—R⁶.

8-11. (canceled)

12. A pharmaceutical composition comprising a compound of general formula (I) according to claim 1 and one or more pharmaceutically acceptable excipients.

13. (canceled)

14. A pharmaceutical composition comprising one or more compounds of general formula (I) according to claim 1 and one or more further anti-cancer agents.

15-19. (canceled)

20. A method of treatment and/or prophylaxis of a disease comprising administering to a subject in need thereof an effective amount of at least one compound of general formula (I) according to claim 1.

21. The method of claim 20, wherein the disease is a hyperproliferative disease.

22. The method of claim 21, wherein the hyperproliferative disease is cancer.

23. The method of claim 22, wherein the cancer is selected from lung cancer, breast cancer, bladder cancer, uterine cancer, endometrial cancer, prostate cancer and leukemia.
